(19) United States
(12) Patent Application Publication
(10) Pub. No.: US 2003/0203372 A1
(43) Pub. Date: Oct. 30, 2003

(54) Analysis method

(76) Inventors: Neil Raymond Ward, Oxford (GB); Christopher Robert Mundy, Oxford (GB); On Kan, Oxford (GB); Robert Alan Harris, Oxford (GB); Jonathan White, Oxford (GB); Katie Mary Binley, Oxford (GB); William Nigel Rayner, Oxford (GB); Stuart Naylor, Oxford (GB); Susan Mary Kingsman, Oxford (GB); David Krige, Oxford (GB)

Correspondence Address: Bruce D. Grant, Morrison & Foerster LLP, Suite 500, 3811 Valley Centre Drive, San Diego, CA, 92130, US

(21) Appl. No.: 10/170,385

(22) Filed: Jun. 12, 2002

Related U.S. Application Data
(63) Continuation-in-part of application No. PCT/GB02/01662, filed on Apr. 8, 2002.
(63) Continuation-in-part of application No. PCT/GB01/05458, filed on Dec. 10, 2001.

(30) Foreign Application Priority Data
Apr. 10, 2001 (GB) GB 0109008.3
Dec. 8, 2000 (GB) GB 0030076.4
Feb. 8, 2001 (GB) GB 0103156.6
Oct. 25, 2001 (GB) GB 0125666.8

(51) Int. Cl. C12Q 1/68

(52) U.S. Cl. 435/6

(57) ABSTRACT

This invention relates to novel methods for the identification of genes and gene products that are implicated in certain disease states. According to the invention, there is provided a method for the identification of a gene that is implicated in a specific disease or physiological condition, said method comprising the steps of comparing: i) the transcriptome or proteome of a first specialized cell type that is implicated in the disease or condition under first and second experimental conditions; with ii) the transcriptome or proteome of a second specialized cell type under said first and said second experimental conditions; and identifying as a gene implicated in the disease or physiological condition, a gene that is differentially regulated in the two specialized cell types under the first and second experimental conditions. The invention also relates to novel genes and gene products identified using these methods.

### CROSS-REFERENCE TO RELATED APPLICATIONS

This application is a continuation-in-part of International patent application PCT/GB02/01662 (filed on Apr. 8, 2002), which is a continuation-in-part of United Kingdom patent application GB 0109008.3 (filed on Apr. 10, 2001), and is a continuation-in-part of International patent application PCT/GB01/05458 (filed on Dec. 10, 2001), the latter being a continuation in part of United Kingdom patent applications GB 0030076.4 (filed on Dec. 8, 2000), GB 0103156.6 (filed on Feb. 8, 2001) and GB 0125666.8 (filed on Oct. 25, 2001). Each of these applications is incorporated by reference in its entirety.

### BACKGROUND OF THE INVENTION

This invention relates to novel methods for the identification of genes and gene products that are implicated in certain disease states. The invention also relates to novel genes and gene products identified using these methods. All publications, patents and patent applications cited herein are incorporated in full by reference.

One of the central goals in the field of gene expression is to understand and elucidate the relationship between a particular disease state and the gene expression pattern that defines and/or causes this disease state. Research has concentrated on differences in expression patterns between diseased and healthy tissues to elucidate the physiological mechanisms of disease. Identified differences in expression patterns provide putative points for therapeutic intervention to reverse the disease phenotype. These differences also provide markers that are useful for diagnosis, and identify proteins for further investigation as agents implicated in the disease in question.

Conventional methods for the elucidation of mechanisms of disease tend to concentrate on the correlation of a disease state with altered levels of a particular protein. Such methods include techniques of immunohistochemistry, the study of differential mRNA expression and the sequence analysis of particular proteins to identify mutations that are associated with a certain disease state.

Recently, research has concentrated on analysis of the transcriptomes of organisms and cell types that are considered to be of scientific interest. By “transcriptome” is meant the exact set of transcripts that are expressed in a cell. The emerging field of nucleic acid arrays is one field in which a large number of powerful tools are being generated for the study of transcriptome variation between different tissue types. These tools are based on techniques originally pioneered by Schena et al., 1995 (Science 270: 467-470) and Fodor et al., 1991 (Science 251, 767-773) and facilitate the evaluation of variations in DNA or RNA sequences and of variations in expression levels from tissue samples and allow the identification and genotyping of mutations and polymorphisms in these sequences. The power of one such technique has recently been demonstrated by Perou et al., (Nature, 2000, 406:747-752), who generated molecular portraits of the transcriptomes of human breast tumours.

Over recent years, the so-called “genomics revolution” has allowed access to large portions of whole genomes, including the human genome. The amount of sequence information now available considerably facilitates the analysis of the results of experiments that aim to elucidate the differences between gene expression in diseased and healthy tissues. As this information increases in scope and becomes more readily available, the study of the molecular mechanism of disease, and the elucidation of techniques for combatting these diseases will be considerably facilitated.

However, there are notable disadvantages associated with all methods that are currently employed for the analysis of human disease. Many methods currently employed utilize established cell lines. Because these cells have been manipulated to allow their immortalization in cell culture, the physiological situation in these cells is not considered by the present inventors to be generally representative of the authentic situation in equivalent cells in vivo. Furthermore, most of these methods tend to utilize a global strategy for intervention, often ignoring the intricacies in gene expression that exist between different tissues. There thus remains a great need for the establishment of novel methods for the analysis of gene expression.

### BRIEF SUMMARY OF THE INVENTION

According to the invention, there is provided a method for the identification of a gene that is implicated in a specific disease or physiological condition, said method comprising the steps of:

a) comparing:

i) the transcriptome or proteome of a first specialized cell type that is implicated in the disease or condition under first and second experimental conditions; with

ii) the transcriptome or proteome of a second specialized cell type under said first and said second experimental conditions; and

b) identifying as a gene implicated in the disease or physiological condition, a gene that is differentially regulated in the two specialized cell types under the first and second experimental conditions.

Using this method, genes have been identified that respond to perturbations of cell physiology in a cell-specific rather than a generic fashion. The method of the invention exhibits significant advantages over conventional methods of identifying genes that are implicated in disease.

Various groups have previously investigated mechanisms of physiological regulation, by comparing gene expression levels in the presence and absence of a physiological stimulus or challenge. Genes identified in a particular cell type as being expressed at different levels under different conditions are implicated as components of a pathway that is responsive to the altered conditions, or that is regulated differently under the altered conditions. However, these methods exhibit a tendency to ignore patterns of gene expression that are physiologically relevant. This inclination is considered to result from a prejudice in the art that dictates that cells respond to changes in certain physiological conditions in a generic fashion, rather than in a cell specific fashion.

By “implicated in a specific disease or physiological condition” is meant that the gene has been found to possess a distinct role in a pathway that is involved in susceptibility to, generation of or maintenance of a particular disease phenotype or physiological condition. As will be apparent to the skilled reader, any point in any pathway may be the unique point at which a cell departs from the normal physiological response and generates a disease phenotype. Often the effect that is manifested as a disease is the result of a mutation event, in which a mutation occurs in the sequence of a gene encoding a protein that functions in a relevant physiological pathway.

There are numerous examples of diseases and conditions that may be studied using the method of the invention. Such pathological conditions include those that result from a change in the intrinsic nature of a cell (usually genetic) or from a change in the cellular microenvironment, either of which might be recapitulated in a laboratory setting. The methods may be applied to any disease or condition that is manifested in, or is generated in a specific cell type.

Examples of such conditions include changes in the cellular microenvironment, exposure to hormones, growth factors, cytokines, chemokines, inflammatory agents, toxins, metabolites, pH, pharmaceutical agents, hypoxia, anoxia, ischemia, imbalance of any plasma-borne nutrient [including glucose, amino acids, co-factors, mineral salts, proteins and lipids], osmotic stress, temperature [hypo and hyper-thermia], mechanical stress, irradiation [ionising or non-ionising], cell-extracellular matrix interactions, cell-cell interactions, accumulations of foreign or pathological extracellular components, intracellular and extracellular pathogens [including bacteria, viruses, fungi and mycoplasma] and genetic perturbations [both epigenetic or mediated by mutation or polymorphism].

Examples of such diseases include cardiovascular disease, atherosclerosis, inflammatory conditions (including rheumatoid arthritis), cancer, ischemic disease, asthma, hematopoietic disorders, neurological diseases including Parkinson's and Alzheimer's diseases, infectious disease and allergies.

One particular physiological response that has been used herein to illustrate the invention is the cellular response to hypoxia. The term “hypoxia” is intended to refer to an environment of reduced oxygen tension, as compared to the normal physiological environment for a particular organism, which is termed “normoxia”. The prejudice in this technical field presents the view that there is a general, ubiquitous response to hypoxia, mediated primarily at the level of mRNA (transcriptional initiation and post-transcriptional stabilisation).

In a variety of human diseases, cells are exposed to conditions of low oxygen tension, usually as a result of poor oxygen supply to the diseased area. For instance, tissue oxygenation plays a significant regulatory role in both apoptosis and in angiogenesis (Bouck et al, 1996, Adv. Cancer Res. 69:135-174; Bunn et al, 1996, Physiol. Rev. 76:839-885; Dor et al, 1997, Trends Cardiovasc. Med., 7:289-294; Carmeliet et al, 1998, Nature 394:485-490). Apoptosis (see Duke et al, 1996, Sci. American, 80-87 for review) and growth arrest occur when cell growth and viability are reduced due to oxygen deprivation. Angiogenesis (i.e. blood vessel growth, vascularization), is stimulated when hypooxygenated cells secrete factors that stimulate proliferation and migration of endothelial cells in an attempt to restore oxygen homeostasis (for review see Hanahan et al, 1996, Cell, 86:353-364).

Ischaemic disease pathologies involve a decrease in the blood supply to a bodily organ, tissue or body part generally caused by constriction or obstruction of the blood vessels. For example, solid tumours typically have a disorganised blood supply, leading to hypoxic regions. Other disease conditions involving hypoxia include stroke, atherosclerosis, retinopathy, acute renal failure, myocardial infarction, stroke and hair loss. Therefore, apoptosis and angiogenesis as induced by the ischaemic condition are also considered to be involved in these disease states. It is generally considered that understanding the mechanism by which cells respond to these diseases may be the key to the disease pathology and thus relevant to disease treatment.

In a different but related approach, it is now recognised that angiogenesis is necessary for tumour growth and that retardation of this process provide a useful tool in controlling malignancy and retinopathies. For example, neoangiogenesis is seen in many forms of retinopathy and in tumour growth. The ability to be able to induce tumourigenic cells to undergo apoptosis is an extremely desirable goal; particularly in the cancer field, it has been observed that apoptosis and angiogenesis-related genes provide potent therapeutic targets. It has also been observed that hypoxia plays a critical role in the selection of mutations that contribute to more severe tumourigenic phenotypes (Graeber et al., 1996 Nature, 379(6560):88-91).

Early in the history of this field it was discovered that a transcription factor, HIF-1alpha, is ubiquitously present in cells and is responsible for the induction of a number of genes in response to hypoxia. This protein is considered a master regulator of oxygen homeostasis (see, for example, Semenza, (1998) Curr. Op. Genetics and Dev. 8:588-594). Where HIF1alpha is genetically knocked out, the hypoxia-inducible transcription of virtually all glycolytic enzymes has been shown to be inhibited. Glycolysis is an essential process which goes on in all mammalian cells. This finding is therefore consistent with previous work showing that when cells are exposed to conditions of hypoxia, they up-regulate glycolytic enzymes to enable ATP production, since oxidative phosphorylation is no longer feasible under conditions of low oxygen (Webster (1987) Mol.Cell.Biochem, 77: 19-28). Further support for a critical and general role of HIF1alpha in the hypoxic response is demonstrated by the knockout mouse, which dies at day 10.5 of gestation. The same is true of the knockout of the ARNT protein, the dimerisation partner of HIF1alpha.

For the first time, it is demonstrated herein that different tissues and cell types exhibit a very different response to hypoxia, at the level of the induction and repression of gene expression. This has allowed the detailed elucidation of the mechanism of this particular physiological response, so paving the way for the development of improved therapeutic agents that target components of the response pathway in particular tissues. Although conventional approaches to the analysis of this mechanism have successfully identified numerous genes, because of the universal prejudice in the art that these components will all be induced/repressed similarly in all cell types, all the approaches suggested have hitherto been limited to the design of therapeutic agents that act in a global fashion.

The methods of the present invention therefore extend and add to previous work performed in this field, in that the discoveries made now allow the design of agents that target the hypoxic response in specific tissues. For example, it is known that brain and heart tissues die very rapidly after ischaemic insult. By using the method of the invention, it is quite possible that these tissues will be found to share common features in their response to hypoxia, that is different from other cell types. This might allow, for example, the design of a combination cardioprotective and neuroprotective agent effective against this subset of body tissues. Alternatively, the hypoxic response in these tissues might be found to be quite different. This information would then be taken into account when designing therapeutic countermeasures, in that an agent would be designed for the unique neurological or cardiological tissue concerned.

The method of the invention involves the comparison of the transcriptomes or proteomes of at least two specialized cell types under two different physiological or genetic conditions. By “transcriptome” is meant the exact set of transcripts that are expressed in a cell. The transcriptome thus has a qualitative element (the identity of individual gene transcripts) and a quantitative element (the proportion of each unique transcript in the total number of individual transcripts present in the cell at a particular moment). By “proteome” is meant the exact set of protein molecules that are expressed in a cell.

By “specialized cell type” is meant a cell type that has a restricted biochemical capacity and that can be unambiguously identified as possessing a unique set of biochemical and physiological functions. Preferably, the specialized cells are primary cells, and not cell lines or whole body tissues. Primary cells are cells that cannot proliferate indefinitely in culture. Primary cells can be derived from adult tissue, or from embryo tissue that is differentiated in culture to an adult cell or to a precursor of an adult cell that displays specialized characteriztics.

Examples of preferred specialized cell types include cardiomyocytes, endothelial cells, sensory neurons, motor neurons, CNS neurons (all types), astrocytes, glial cells, schwann cells, mast cells, eosinophils, smooth muscle cells, skeletal muscle cells, pericytes, lymphocytes, tumor cells, monocytes, macrophages, foamy macrophages, granulocytes, synovial cells/synovial fibroblasts, epithelial cells (varieties from all tissues/organs). Examples of other suitable specialized cell types include vascular endothelial cells, smooth muscle cells (aortic, bronchial, coronary artery, pulmonary artery, etc), skeletal muscle cells, cardiomyocyte cells, fibroblasts (many types, such as synovial), keratinocytes, hepatocytes, dendritic cells, astrocytes, neuron cells (including mesencephalic, hippocampal, striatal, thalamic, hypothalamic, olfactory bulb, substantia nigra, locus coeruleus, cortex, dorsal root ganglia, superior cervical ganglia, sensory, motor, cerebellar cells), neutrophils, eosinophils, basophils, mast cells, monocytes, macrophage cells, erythrocytes, megakaryocytes, hematopoietic progenitor cells, hematopoietic pluripotent stem cells, any stem cells, any progenitor cells, epithelial cells, melanocytes, osteoblasts, osteoclasts, stromal cells, purkinje cells, T-cells, B-cells, synovial cells, pancreatic islet cells (alpha and beta), leukemia cells, lymphoma cells, tumour cells, retinal cells, adrenal chromaffin cells. As will be apparent to the skilled reader, it is not here possible to provide an exhaustive list of specialized cell types that may be studied according to the methods of the present invention.

Intended as being included within the method of the invention is the possibility of using, as two different specialized cell types, two different physiological states of the same cell type, for example, activated and resting macrophages.

The transcriptomes of the specialized cell types are compared under different experimental conditions. The term “experimental conditions” is used broadly in this context and is intended to embrace any physiological or genetic conditions to which a cell type may be exposed. The intention of the method is to compare the transcriptomes or proteomes of the cell types under different experimental conditions that have a physiological relevance. Accordingly, the state of the transcriptome or proteome under one set of experimental conditions will generally act as a control against which the transcriptome or proteome may be compared under a second set of experimental conditions. Any distinct physiologically-relevant conditions may therefore be of interest.

Examples of suitable physiological experimental conditions include conditions under which the cell is submitted to a physiological, mechanical, temperature, chemical, toxic or pharmaceutical stress. One example is hypoxia, defined herein as a physiological state in which oxygen demand by the cell exceeds its supply to the cell. The transcriptome or proteome under this set of experimental conditions may be compared to the transcriptome or proteome under conditions of normoxia, when oxygen supply is in concordance with the demand by the cell.

The transcriptomes or proteomes may also be compared under different genetic conditions. By “genetic conditions” is meant that the genotype of the compared cell populations contains a different genetic component. This may be the presence of one or more different, non-endogenous nucleic acid molecules in the cell, herein referred to collectively as “genetic elements”. Such genetic element(s) may potentially be incorporated into the genome of the cell, or alternatively may exist as a separate genetic entity, for example, as an extra-chromosomal element such as a plasmid or episome. Alternatively, the genome may have been perturbed by external intervention, for example, to increase or decrease the expression of a particular gene or genes. A number of variations on this theme are possible, including the overexpression of a genetic element via the administration of the functional gene, the overexpression of a genetic element via the administration of a regulator of the functional gene (such as, for example, a transcription factor [either natural or artificially constructed via the fusion of a DNA binding domain with an activator domain]), the inhibition of the expression of a functional gene (for example, using antisense RNA or ribozymes), the inhibition of the expression of a functional gene (for example, using a transdominant protein) and the inhibition of the expression of a functional gene (for example, using a repressor protein that is either natural or artificially constructed from a DNA binding protein fused to a repressor domain).

A particular example of a genetic perturbation as envisaged herein, that forms one preferred embodiment of the method of the present invention, is the so-called “Smartomics” technology that forms the basis for co-pending, co-owned International patent application PCT/GB01/00758. According to this technology, a heterologous nucleic acid is introduced into a primary cell to augment a specific natural physiological response. “Smartomics” may be applied to the current invention by measuring and comparing cellular responses to a heterologous gene in two or more distinct cell types, both with and without the natural physiological stimulus. Lentivirus technology is used to introduce the heterologous nucleic acid molecule in such a way that there is negligible perturbation of endogenous gene expression. For this reason, this technology exhibits significant benefits over conventional technology of a similar nature, since the prior art methods are generally invasive, having downstream effects other than the simple introduction of the heterologous nucleic acid molecule. The Smartomics technology allows much more precise measurements to be taken of the effect of introducing the heterologous nucleic acid.

The method of the invention allows the identification of genes that are implicated in a specific disease or physiological condition. The genes identified in this way are candidate targets for antagonists or agonists that modulate disease states pertinent to that specialized cell type. This allows the development of selective agonists and antagonists, rather than broad spectrum agonists and antagonists. This approach thus adds value in the selective treatment of disease. Furthermore the identified genes are associated with regulatory elements that provide alternative and additional candidate targets for exploitation for the delivery of gene products to that cell in a cell-specific fashion. The genes and regulatory elements identified according to the method of the invention can be used directly in therapeutic applications via gene therapy, via recombinant protein methods or via chemical mimetics or as targets for the development of agonists and antagonists such as antibodies, intrabodies, small chemical molecules, peptides, regulatory nucleic acids.

The step of comparison of the transcriptomes or proteomes of the first and second specialized cell types under first and second experimental conditions may be effected using any approach that allows the quantitative comparison of gene or protein expression, and a number of such means will be known to those of skill in the art. Such experiments have only become possible in recent years, due to certain advances in technology that have allowed the large scale, high throughput analysis of gene expression.

One example of a method that allows the comparison of the transcriptome of a specific cell type with a second or subsequent transcriptome involves the generation of a set of clones that represent all the transcripts expressed in a cell under the conditions in which the cell is maintained. This may be done by constructing a cDNA library, in which copies of all mRNA transcripts expressed in the cell are cloned into a suitable vector for subsequent analysis.

Such libraries may be normalized cDNA libraries, in which the distribution of genes in the library has been biased to reduce the number of clones that represent genes with large numbers of transcripts (such as, for example, beta-actin) and thus reduce the repetitive nature of the library. Normalisation thus acts to reduce the frequency of genes expressed at high levels and to enhance the frequency of genes expressed at low levels (see de Fatima Bonaldo et al., Genome Research 6: 791-806 (1996)).

Libraries may also be subtracted cDNA libraries, in which the distribution of genes is manipulated to remove genes that are expressed in both mRNA populations used to construct the library. The commercially-available PCR Select kit (Clontech, Inc) is an example of a system useful to generate such libraries.

cDNA clones generated as reflective of the transcriptome of a specific cell type may then be amplified, and processed to evaluate the identity of the nucleic acid clones. For example, multiple clones may be picked and used as template for PCR amplification. The PCR products may then be arrayed onto membranes or glass slides to create nucleic acid arrays. For expression profiling, these arrays are then hybridised to complex nucleic acid probes in order to quantitate the abundance of individual genes contained in the probes.

A recent summary of nucleic acid array technology that is useful in the analysis of the transcriptome of a cell population is provided in Nature Genetics, (1999) (21 suppl; 1-61). There are various types of array technology currently used, including “microarrays”, or “chips”, which are high density cDNA arrays produced on glass slides, often produced using photolithography. A second type of array is the “macroarray”, which is an array with sub-millimetre spot-spot distances produced on a nylon membrane. One example of this type of array are the nylon-based microarrays sold commercially by Research Genetics Inc. (termed Research Genetics Human GeneFilters) that each contain 5,300 cDNA fragments of known identity. The whole series of arrays covers some 35,000 cDNA fragments. This particular array system (and others like it) allow the identification of transcripts that are down-regulated, as well as those that are up-regulated, since the range of genes used to manufacture the arrays are not biased.

The step of comparison may be effected by utilising subtracted cDNA libraries. Using this approach, the transcriptome of one specialized cell type under first experimental conditions is subtracted against the transcriptome under second experimental conditions. This reveals the differences in expression under the two experimental conditions tested. When this is performed for both specialized cell types, the differential regulation of gene expression under the two experimental conditions is revealed.

The step of comparison is through the detection of genes that are differentially regulated in the two specialized cell types examined under the first and second experimental conditions. As an example, a human cardiomyoblast (cell type A) and a human macrophage (cell type B) may be placed at the same temperature and at a high oxygen tension (first experimental conditions [1]). Cells from the same cell types are also incubated at this temperature, yet under conditions of low oxygen tension (second experimental conditions [2]). In this simple example, there are then a minimum of four combinations of cell type and condition, A[1], B[1], A[2] and B[2]. “Snapshots” are taken of the transcriptomes of both cell types under the “normoxic” and the “hypoxic” experimental conditions, by preparing messenger RNA from all four combinations. Differences in the regulation of genes can then be analysed, for example, using a process of subtractive hybridization.

The mechanism of transcriptome comparison in the above example may be as follows. Subtracted cDNA libraries are separately prepared for hypoxic macrophages and cardiomyoblasts; for both cell types, their cDNA under normoxic conditions is subtracted against their cDNA under hypoxic conditions. This might be effected by harvesting RNA from cells both in normoxia and hypoxia, and preparing cDNA. Subtractive hybridization, optionally including suppression PCR, may then be performed to remove genes from the hypoxic cell cDNA which are also present in cDNA from normoxic cells. Insert DNA from these subtracted libraries can then be amplified and arrayed onto duplicate membranes. Quantitative hybridization with pre-library cDNA material (normoxia and hypoxia) then allows the comparison of differentially-expressed clones in the two cell types. The clones representing hypoxia-inducible genes may be then be identified, for example, by sequencing.

Other techniques that are suitable for the analysis of the transcriptome of a specific cell type include serial analysis of gene expression (SAGE; Velculescu et al., Science (1995) 270; 484-487), Selective amplification via biotin- and restriction-mediated enrichment (SABRE) (Lavery et al, (1997), PNAS USA 94: p6831-6836); Differential display (for example, indexing differential display reverse transcriptase polymerase chain reaction (DDRT-PCR; Mahadeva et al. (1998) J. Mol.Biol. 284, 1391-1398)); representational difference analysis (RDA) (Hubank (1999) Methods in Enzymology 303: 325-349); differential screening of cDNA libraries (see Sagerstrom et al. (1997) Annu. Rev. Biochem. 66: 751-783); “Advanced Molecular Biology”, R. M. Twyman (1998) Bios Scientific Publishers, Oxford; “Nucleic Acid Hybridization”, M. L. M. Anderson (1999) Bios Scientific Publishers, Oxford); Northern blotting; RNAse protection assays; S1-nuclease protection assays; RT-PCR; real time RT-PCR (Taq-man); EST sequencing; massively parallel signature sequencing (MPSS); and sequencing by hybridization (SBH) (see Drmanac R. et al (1999), Methods in Enzymology 303:165-178). Many of these techniques are reviewed in “Comparative gene-expression analysis” Trends Biotechnol. February 1999;17(2):73-8.

Methods such as these have been applied widely to study mechanisms of biological response. In particular, microarrays have been used widely to compare gene expression levels between normal and diseased tissue. More typically, however, comparisons are performed to detect changes in gene expression that are associated with specific aspects of disease progression or pathology. For instance, a study of prostate cancer would examine changes associated with the step-wise progression to full malignancy or the dependence on androgens for growth.

Transcriptome analysis is complemented by the analysis of the complete protein make-up of a cell, referred to as proteomics. The use of two dimensional SDS-PAGE gels in combination with amino acid sequencing by mass spectrometry is currently the most widely-used technique in this field (see “Proteomics to study genes and genomes” Akhilesh Pandey and Matthias Mann, (2000), Nature 405: 837-846). Additionally, the recent developments in the field of protein and antibody arrays now allow the simultaneous detection of a large number of proteins. For example, low-density protein arrays on filter membranes, such as the universal protein array system (Ge H, (2000) Nucleic Acids Res. 28(2), e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in patients pre- and post-drug treatment.

Antibody arrays also facilitate the extensive parallel analysis of numerous proteins that are hypothetically implicated in a disease or particular physiological state. A number of methods for the preparation of antibody arrays have recently been reported (see Cahill, Trends in Biotechnology, 2000 7:47-51).

It is not the intention here to review studies that have been conducted in this area previously. However, one example of a physiological condition that has already received considerable attention is the response to hypoxia. Several patent applications have now been published that involve an examination of the genetic response to hypoxia (see WO00/12139, Quark Biotech, Inc.; WO00/12525, Quark Biotech, Inc.; WO99/09049, Quark Biotech, Inc.; WO99/09046, Quark Biotech, Inc.; WO99/48916, The Board of Trustees of the Leland Stanford Jr. University). These patent applications generally utilize methods of subtractive hybridization and differential expression gene microarray analysis to examine this genetic response in certain cell lines. The studies have implicated specific genes as being either repressed or induced under hypoxic conditions as compared to their expression under normoxic conditions. These genes are taught as being useful generally in all cell types, being involved in the (generic) hypoxic response.

Significantly, the present invention extends this work, and, indeed, defines a significant advance over similar work that has been performed on the genetic mechanisms that act in response to other physiological or genetic stimuli. The present inventors, using the novel methods disclosed herein, have discovered that far from being generic, the cellular response to many physiological conditions differs markedly between different cell types. The cellular response that has been studied in order to illustrate this finding is the response to hypoxia. From these results, it has been inferred herein, quite reasonably, that far from being generic, cellular response mechanisms differ widely, depending on cell type.

This discovery has far-reaching implications as regards the design of therapeutic agents that are effective to counter a disease or physiological condition. For example, an agent that is effective to prevent the drastic effects of hypoxia in a neuron (the effects of which include stroke) might be totally ineffective in countering the same effects in a cardiomyocyte (chronic ischemic heart disease). Through analysing the mechanism of the hypoxic response in different cell types, it may be, in contrast to the example given above, that a particular gene is involved in the hypoxic response in both cardiomyocytes and neurons. Were this to be the case, this would allow the design of a combined medicament, for example, a combined cardioprotective and neuroprotective agent.

As discussed in some detail above, ischaemic disease pathologies involve a decrease in the blood supply to a bodily organ, tissue or body part generally caused by constriction or obstruction of the blood vessels. One particular example of an ischaemic disease pathology is myocardial ischaemia, which encompasses several chronic and acute cardiac pathologies that involve the deprivation of the myocardium of its blood supply, usually through coronary artery occlusion. A key component of ischaemia is hypoxia. Following transient ischaemia, the affected tissue may be subjected to reperfusion and re-oxygenation, and this is of significance in its own right.

Ischaemia/reperfusion is well known to induce cell death in myocardial tissue by apoptosis, leading to impaired fuinction of the myocardium and infarction. Many of the specific molecules required to execute the process of apoptosis are known, but not all of these molecules have been characterized in detail. Cell death may also proceed by a distinct process called necrosis, which unlike apoptosis, is not initiated and controlled by specific and dedicated cellular and biochemical mechanisms (see Nicotera et al., Biochem Soc Symp. 1999; 66:69-73). There is substantial evidence that apoptotic cell death occurs either during or after myocardial ischaemia (Kajstura et aL, Lab Invest. 1996; 74(1):86-107; Cheng et al., Exp Cell Res. 1996; 226(2):316-27; Fliss and Gattinger, Circ Res. 1996; 79(5):949-56; Veinot et al., Hum Pathol. 1997; 28(4):485-92; Bialik et al., J Clin Invest. 1997; 100(6):1363-72; Gottlieb et al., J Clin Invest. 1994; 94(4):1621-8; Gottlieb and Engler, Ann N Y Acad Sci. 1999; 874:412-26). In the laboratory, apoptosis is also induced by subjecting cardiac myocytes to hypoxia (Tanaka et al., Circ Res. September 1994;75(3):426-33; Long et al., J Clin Invest. 1997 99(11): 2635-43).

Clearly, there is a significant clinical application were a successful method to inhibit apoptosis in ischaemic myocardial tissue to be devised. A specific and effective treatment requires identifying biochemical target(s), which are responsible for mediating apoptosis, specifically in ischaemic myocardial cells. One target which plays a common role in mediating apoptosis in many cell types, namely p53, is not involved in apoptosis resulting from myocardial ischaemia (Bialik et al., J Clin Invest. 1997; 100(6):1363-72). Others have shown that inhibiting key mediators of apoptosis, caspases, provides protection against lethal reperfusion injury, following myocardial ischaemia in rat models (Mocanu et al., Br J Pharmacol. 2000; 130(2):197-200; Yaoita et al., Circulation. 1998 97(3): 276-81; Holly et al., J Mol Cell Cardiol. 1999 31(9): 1709-15). However, this approach lacks specificity, since the caspases play a key role in mediating apoptosis in the majority of manmmalian cell types, where it is usually beneficial. An approach that involves modulating the activity of molecules shown specifically to mediate apoptosis in ischaemic cardiac cells, would present a distinct advantage in both specificity and efficacy.

There thus remains a great need for the identification of proteins implicated in the physiological mechanism of hypoxia, in order to develop a spectrum of diagnostic and therapeutic agents for use as tools in combating diseases in which hypoxia plays a role. Such genes and the proteins that they encode are candidate targets for antagonist or agonist agents that modulate human disease states. Furthermore, the identified genes are associated with regulatory elements that provide alternative and additional candidate targets for exploitation for the delivery of gene products in a cell-specific fashion. Any genes and regulatory elements identified as having a role in hypoxia may be used directly in therapeutic applications via gene therapy, via recombinant protein methods or via chemical mimetics or as targets for the development of agonists and antagonists such as antibodies, intrabodies, small chemical molecules, peptides, regulatory nucleic acids.

Novel Targets

According to a further aspect of the invention, there are provided genes and proteins that are identified using a method according to any one of the above-described aspects of the invention. Certain proteins, whose sequences are identified herein as SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531, are functionally annotated for the first time. At present, all of these sequences are only identified as “hypothetical proteins” in the public databases. Each and every one of these sequences forms an embodiment of this aspect of the invention.

The invention also includes proteins whose amino acid sequences are encoded by a nucleic acid sequence recited in various cDNAs and ESTs deposited in the public databases, or encoded by a gene identified from such an EST. These cDNAs and ESTs are presented herein as SEQ ID NOS: 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532. At present, all of these cDNA and EST sequences are functionally unannotated in the public databases. Each and every one of these sequences forms an embodiment of this aspect of the invention.

One embodiment of this aspect of the invention provides substantially purified polypeptide, which polypeptide:

i) comprises the amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 or 531;

ii) has an amino acid sequence encoded by a nucleic acid sequence recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532 or encoded by a gene identified from an EST recited in any one of these SEQ ID NOS;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

The polypeptide sequences recited in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531 were, prior to the present disclosure, unannotated in the literature and public sequence databases. Accordingly, until now, no biological function has been attributed to these polypeptide sequences; each of these sequences is generally labelled in the databases as a “hypothetical protein”. The methods of the present invention, described above, have now elucidated a biological function for these polypeptides, in that they have been found to be differentially regulated under physiological conditions of hypoxia.

These discoveries allow the development of regulators, such as small drug molecules, that affect the activity of these polypeptides, so allowing diseases and physiological conditions that are caused by hypoxia, or in which hypoxia has been implicated, to be treated. These discoveries also allow the development of diagnostic agents that are suitable for the detection of hypoxia in biological tissues and, through the identification of mutations and polymorphisms (such as SNPs) within genes coding for the proteins implicated herein, allows the assessment of an individual's risk of being susceptible to diseases and physiological conditions in which hypoxia is implicated.

The biological activity of polypeptides whose sequences are listed in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531 has been found to be hypoxia-regulated. The expression of some of these polypeptides has been found to be induced under conditions of hypoxia, whilst the expression of other polypeptides has been found to be repressed. By “hypoxia-induced” is meant that the polypeptide is expressed at a higher level when a cell is exposed to hypoxic conditions as compared to its expression level under normoxic conditions. By “hypoxia-repressed” is meant that the polypeptide is expressed at a lower level when a cell is exposed to hypoxic conditions as compared to its expression level under normoxic conditions.

The following polypeptides have been found to be hypoxia-induced: those polypeptides whose amino acid sequence is recited in SEQ ID NOS: 1, 3, 5, 7, 9, 11, 13, 15, 17, 19, 21, 23, 25, 27, 29, 31, 33, 63, 67, 69, 73, 75, 77, 85, 85a, 87, 89, 91, 93, 95, 99, 103, 113, 115, 119, 121, 129, 131, 133, 137, 139, 141, 527, 529 and 531; and those polypeptides whose amino acid sequence is encoded by a nucleic acid sequence recited in SEQ ID NOS: 2, 4, 6, 8, 10, 12, 14, 16, 18, 20, 22, 24, 26, 28, 30, 32, 34, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 528, 530 and 532 or is encoded by a gene identified from an EST recited in any one of these SEQ ID NOS.

The following polypeptides have been found to be hypoxia-repressed: those polypeptides whose amino acid sequence is recited in SEQ ID NOS: 35, 37, 39, 41, 43, 45, 47, 49, 51, 53, 55, 57, 59, 145, 151, 153, 157, 159, 163, 169, 181, 187, 201, 205, 207 and 209; and those polypeptides whose amino acid sequence is encoded by a nucleic acid sequence recited in SEQ ID NOS: 36, 38, 40, 42, 44, 46, 48, 50, 52, 54, 56, 58, 60, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214 and 216, or encoded by a gene identified from an EST recited in any one of these SEQ ID NOS.

For the purposes of this document, the term “hypoxia” should be taken to mean an environment of oxygen tension such that the oxygen content is between about 5% and 0.1% (v/v). In most cases, hypoxic tissue will have an oxygen content that is less than or equal to about 2%. The term “normoxia” should be taken to mean conditions comprising a normal level of oxygen for the environment concerned. Normoxic tissue typically has an oxygen content above about 5%.

The polypeptide sequences whose amino acid sequence is encoded by a nucleic acid sequence recited in SEQ ID NOS: 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, or whose amino acid sequence is encoded by a gene identified from an EST recited in any one of these SEQ ID NOS, were also, prior to the present disclosure, unannotated in the literature and public sequence databases, meaning that until now, no biological function has been attributed to these polypeptide sequences.

The sequences in this group fall into a number of different categories. The first of these are cDNA clones, for which a protein sequence has not been predicted by the depositor. A second category is expressed sequence tag (EST) sequences that are represented in the UniGene database (http address www.ncbi.nlm.nih.gov/UniGene/), which contain modest or weak homology to known proteins when translated. ESTs are single-pass sequence files of the 5′ region of an organism's expressed genome as accessed via a force cloned cDNA library. EST sequences tend to be short and as a general rule are error-prone. UniGene (see http address www.ncbi.nlm.nih.gov/Web/Newsltr/aug96.html for review) is an experimental system for automatically partitioning these EST sequences into a non-redundant set of gene-oriented clusters. Each UniGene cluster contains sequences that represent a unique gene, as well as related information such as the tissue types in which the gene has been expressed and map location. A third category of hits identified by the methods described herein is EST sequences that are contained in Unigene clusters, but which are not annotated and exhibit no homologies to proteins contained in the public databases. The fourth and final category encompasses singleton EST sequence entries that are not incorporated as entries in the Unigene database and that only appear as single entries in the public databases.

The methods of the present invention, described above, have now elucidated a biological function for polypeptides that are encoded by genes incorporating cDNA and EST sequences that fall into the four categories set out above, in that these sequences have been found to be differentially regulated under physiological conditions of hypoxia. Such polypeptides may have an amino acid sequence that is encoded by a nucleic acid sequence recited in any one of SEQ ID NOS: 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214 and 216. However, the EST sequences in particular may not be part of the actual coding sequence for a gene, often representing regulatory regions of the gene, or regions that are transcribed, but not translated into polypeptide. Accordingly, this aspect of the invention also includes polypeptides that are encoded by a gene identified from an EST recited in any one of SEQ ID NOS: 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214 and 216.

SM20 Homologues

It has now been discovered that a polypeptide encoded by a gene identified from the EST recited in SEQ ID No 86, having the Protein accession number BAB15101; herein referred to as SEQ ID NO: 85 (encoded by Homo sapiens cDNA: FLJ21620 fis, clone COL07838 Nucleotide accession AK025273) is regulated by hypoxia. Other public domain sequences corresponding to this gene include Homo sapiens cDNA: FLJ23265 fis, clone COL06456 Nucleotide accession AK026918. Accordingly, when referring in the present specification to the EST recited in SEQ ID No 86, it is intended that these gene and protein sequences are also embraced. This gene was identified using Research Genetics Human GeneFilters arrays, which contain an EST corresponding to the gene (accession number R00332) In the art, the gene is now termed EGL nine (C.elegans) homolog 3.

There are no reports that describe the function of this human gene. However, a high degree of amino acid homology is observed between the protein encoded by this gene, and a rat protein called “Growth factor responsive smooth muscle protein” or “SM20” (Nucleotide accession U06713; Protein accession A53770). An alignment of single letter amino acid sequences is shown below. Over the italicized region there is 97% amino acid similarity and 96% amino acid identity.

| | | |
|---|---|---|
| A53770 | (1) | MTLRSRRGFLSFLPGLRPPRRWLRISKRGPPTSHWASPALGGRTLHTSCR |
| BAB15101 | (1) | -------------------------------------------------- |
| | | 51 100 |
| A53770 | (51) | SQSGTPFSSEFQATFPAFAAKVARGPWLPQVVEPPARLSASPLCVRSGQA |
| BAB15101 | (1) | -------------------------------------------------- |
| | | 101 150 |
| A53770 | (101) | LGACTLGVPRLGSVSEMPLGHIMRLDLEKIALEYIVPCLHEVGFCYLDNF |
| BAB15101 | (1) | ----------------MPLGHIMRLDLEKIALEYIVPCLHEVGFCYLDNF |
| | | 151 200 |
| A53770 | (151) | LGEVVGDCVLERVKQLHYNGALRDGQLAGPRAGVSKRHLRGDQITWIGGN |
| BAB15101 | (35) | LGEVVGDCVLERVKQLHCTGALRDGQLAGPRAGVSKRHLRGDQITWIGGN |
| | | 201 250 |
| A53770 | (201) | EEGCEAINFLLSLIDRLVLYCGSRLGKYYVKERSKAMVACYPGNGTGYTVR |
| BAB15101 | (85) | EEGCEAISFLLSLIDRLVLYCGSRLGKYYVKERSKAMVACYPGNGTGYVR |
| | | 251 300 |
| A53770 | (251) | HVDNPNGDGRCITCIYYLNKNWDAKLHGGVLRIFPEGKSFVADVEPIFDR |
| BAB15101 | (135) | 301 350 |
| AS5770 | (301) | LLFSWSDRRNPHEVQPSYATRYAMTVWYFDAEERAEAKKKFRNLTRKTES |
| BAB15101 | (185) | LLFFWSDRRNPHEVQPSYATRYAMTVWYFDAEERAEAKKKFRNLTRKTES |
| | | 351 |
| A53770 | (351) | ALAKD |
| BAB15101 | (235) | ALTED |

The high degree of amino acid similarity suggests that the human protein BAB15101 has an equivalent biochemical function to the rat protein A53770 (“Growth factor responsive smooth muscle protein” or “SM20”). Recent publications have shown that SM20 functions to promote apoptosis in neurons (Lipscomb et al., J Neurochem 1999; 73(1):429-32; Lipscomb et al., J Biol Chem. Feb. 16, 2001;276(7):5085-92). Significantly, SM20 has been shown to be expressed at high levels in the heart (Wax et al., J Biol Chem 1994; 269(17): 13041-7). This aspect of the invention therefore provides a substantially purified prolyl 4-hydroxylase polypeptide, which polypeptide:

i) comprises the amino acid sequence recited in SEQ ID NO: 85;

ii) has an amino acid sequence encoded by a nucleic acid sequence recited in SEQ ID NO: 86;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

It has also been discovered that there is a so far undisclosed splice variant of the polypeptide sequence recited in SEQ ID NO: 85. The polypeptide sequence recited in SEQ ID NO: 85a is a novel isoforn of the polypeptide sequence recited in SEQ ID NO: 85. The novel cDNA isoform of FLJ21620 fis, clone COL07838, lacks an internal stretch of nucleotides, probably due to an alternative splicing event. The nucleotide sequence of the sequenced portion of the new variant is shown below, and is referred to herein as SEQ ID No. 86a:

| | |
|---|---|
| 1 | CTCGATTCTG CGGGCGAGAT GCCCCTGGGA CACATCATGA GGCTGGACCT |
| 51 | GGAGAAAATT GCCCTGGAGT ACATCGTGCC CTGTCTGCAC G*AGGCAATGG |
| 101 | TGGCTTGCTA TCCGGGAAAT GGAACAGGTT ATGTTCGCCA CGTGGACAAC |
| 151 | CCCAACGGTG ATGGTCGCTG CATCACCTGC ATCTACTATC TGAACAAGAA |
| 201 | TTGGGATGCC AAGCTACATG GTGGGATCCT GCGGATATTT CCAGAGGGGA |
| 251 | AATCATTCAT AGCAGATGTG GAGCCCATTT TTGACAGACT CCTGTTCTTC |
| 301 | TGGTCAGATC GTAGGAACCC ACACGAAGTG CAGCCCTCTT ACGCAACCAG |
| 351 | ATATGCTATG ACTGTCTGGT ACTTTGATGC TGAAGAAAGG GCAGAAGCCA |
| 401 | AAAAGAAATT CAGGAATTTA ACTAGGAAAA CTGAATCTGC CCTCACTGAA |
| 451 | GAC |

The part of FLJ21620 fis, clone COL07838 which is missing in the above sequence at the position indicated by an asterisk, is shown below:

| | |
|---|---|
| 1 | AGGTGGGCTT CTGCTACCTG GACAACTTCC TGGGCGAGGT GGTGGGCGAC |
| 51 | TGCGTCCTGG AGCGCGTCAA GCAGCTGCAC TGCACCGGGG CCCTGCGGGA |
| 101 | CGGCCAGCTG GCGGGGCCGC GCGCCGGCGT CTCCAAGCGA CACCTGCGGG |
| 151 | GCGACCAGAT CACGTGGATC GGGGGCAACG AGGAGGGCTG CGAGGCCATC |
| 201 | AGCTTCCTCC TGTCCCTCAT CGACAGGCTG GTCCTCTACT GCGGGAGCCG |
| 251 | GCTGGGCAAA TACTACGTCA AGGAGAGGTC TA |

At the protein level the new variant forms a functional protein, and retains the open reading frame, as shown below (SEQ ID No.:85a). This sequence starts and finishes with the same amino acid sequence but is missing an internal portion compared with the translation of FLJ21620 fis, clone COL07838.

| | |
|---|---|
| 1 | MPLGHIMRLD LEKIALEYIV PCLHEAMVAC YPGNGTGYVR HVDNPNGDGR |
| 51 | CITCIYYLNK NWDAKLHGGI LRIFPEGKSF IADVEPIFDR LLFFWSDRRN |
| 101 | PHEVQPSYAT RYAMTVWYFD AEERAEAKKK FRNLTRKTES ALTED |

This aspect of the invention therefore provides a substantially purified prolyl 4-hydroxylase polypeptide, which polypeptide:

i) comprises the amino acid sequence recited in SEQ ID NO: 85a;

ii) has an amino acid sequence encoded by a nucleic acid sequence recited in SEQ ID NO: 86a;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

It has also been discovered that a polypeptide encoded by a gene identified from the EST recited in SEQ ID No 90, having the Protein accession number CAB81622, is regulated by hypoxia. The encoding human gene has been annotated in the UniGene database as “Similar to rat smooth muscle protein SM-20”; the nucleotide sequence is contained within the nucleotide accession AL117352. More recently, a longer fragment of this gene has been cloned, named c1orf12, or EGLN1 (Nucleotide accession AAG34568; Protein accession AAG34568). Accordingly, when referring in the present specification to the EST recited in SEQ ID No 90, it is intended that these gene and protein sequences are also embraced.

This distinct human gene, encoding a protein related to SM20 and EGLN3 (BAB15101), is also induced in response to hypoxia. This gene was identified using Research Genetics Human GeneFilters arrays, which contain an EST corresponding to the gene (accession number H56028). The protein sequence, SEQ ID No 89, is given below:

| | |
|---|---|
| 1 | MANDSGGPGG PSPSERDRQY CELCGKNENL LRCSRCRSSF YCCKEHQRQD WKKHKLVCQG |
| 61 | SEGALGHGVG PHQHSGPAPP AAVPPPRAGA REPRKAAARR DNASGDAAKG KVKAKPPADP |
| 121 | AAAASPCRAA AGGQGSAVAA EAEPGKEEPP ARSSLFQEKA NLYPPSNTPG DALSPGGGLR |
| 181 | PNGQTKPLPA LKLALEYIVP CMNKHGICVV DDFLGKETGQ QIGDEVRALH DTGKFTDGQL |
| 241 | VSQKSDSSKD IRGDKITWIE GKEPGCETIG LLMSSMDDLI RHCNGKLGSY KINGRTKAMV |
| 301 | ACYPGNGTGY VRHVDNPNGD GRCVTCIYYL NKDWDAKVSG GILRIFPEGK AQFADIEPKF |
| 361 | DRLLFFWSDR RNPHEVQPAY ATRYAITVWY FDADERARAK VKYLTGEKGV RVELNKPSDS |
| 421 | VGKDVF |

This aspect of the invention therefore provides a substantially purified prolyl 4-hydroxylase polypeptide, which polypeptide:

i) comprises the amino acid sequence recited in SEQ ID NO: 89;

ii) has an amino acid sequence encoded by a nucleic acid sequence recited in SEQ ID NO: 90;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

Independently to this, a fragment of this gene has been cloned from a cDNA library derived from hypoxic human cardiomyoblasts, and it has been shown that the gene is increased in expression in response to hypoxia in this cell type (see Table 1 herein; penultimate row). The nucleotide sequence of this cDNA fragment, referred to herein as SEQ ID No 90a is:

| | |
|---|---|
| 1 | ACCTCTACAG TTGTAAAAAG TATTAGATTC TACTATCTGT GGGTTGTGCT TGCCAGACAG |
| 61 | GTCTTAAATT GTATATTTTT TGGAAAAGTT TATATACTCT CTTAGGAATC ATTGTGAAAA |
| 121 | GATCAAGAAA TCAGGATGGC CATTTATTTA ATATCCATTC ATTTCATGTT AGTGGGACTA |
| 181 | TTAACTTGTC ACCAAGCAGG ACTCTATTTC AAACAAAATT TAAAACTGTT TGTGGCCTAT |
| 241 | ATGTGTTTAA TCCTGGTTAA AGATAAAGCT TCATAATGCT GTTTTTATTC AACACATTAA |
| 301 | CCAGCTGTAA AACACAGACC TTTATCAAGA GTAGGCAAAG ATTTTCAGGA TTCATATACA |
| 361 | GATAGACTAT AAAGTCATGT AATTTGAAAA GCAGTGTTTC ATTATGAAAG AGCTCTCAAG |
| 421 | TTGCTTGTAA AGCTAATCTA ATTAAAAAGA TGTATAAATG TTGTCAAAAA AAAAAAAAAA |
| 481 | AAAAGAAAAA AAGT |

In the light of this novel discovery reported herein that these human equivalents of SM20 are induced by hypoxia, it is herein proposed that in cardiac ischaemia, the resulting apoptosis is due at least in part, to increased expression of these genes.

The therapeutic modulation of the activity of EGLN3 (BAB15101; SEQ ID NO: 85), the EGLN3 splice variant (SEQ ID NO: 85a), EGLN1 (c1orf12; AAG34568; SEQ ID NO: 89), CAB81622, SM20 and other equivalent proteins and encoding genes therefore provides a novel means for the treatment of myocardial ischaemia, through the alteration of the propensity of myocardial cells to undergo apoptosis. For example, a suitable treatment may involve altering the susceptibility of ischaemic myocardial tissue to subsequent reperfusion and re-oxygenation, or may involve modulating the susceptibility of chronic ischaemic myocardial tissue (including forms of angina) to later more severe ischaemia, which would result in myocardial infarction. It is submitted that, by way of analogy, cerebral ischaemia may be treated using the same principle.

These data provide the first connection between these related genes and the physiological response to hypoxia. Recently published research papers have identified that the protein products of these genes can act as proline hydroxylases (see Bruick RK et al Science. 2001 294:1337-40 and Epstein AC et al Cell. 107:43-54). This is consistent with our observations that certain proline hydroxylases are induced in response to hypoxia and the genes EGLN1 and EGLN3 are part of the hypoxia response. For example, two genes encoding proline hydroxylases have been identified herein as being increased in expression in response to hypoxia (proline 4-hydroxylase, alpha polypeptide 1; SeqID: 231/232, proline 4-hydroxylase, alpha polypeptide II; SeqID: 349/350). This identified a functional significance of proline hydroxylation as a response to hypoxia. A preferred embodiment of the invention thus includes methods for modulating the biological response to hypoxia by modulating the proline hydroxylase activity of the EGLN3 (BAB15101; SEQ ID NO: 85), the EGLN3 splice variant (SEQ ID NO: 85a) c1orf12 (AAG34568; SEQ ID NO: 89), CAB81622, SM20 and other equivalent proteins.

Furthermore, a number of bacteria, such as moraxella, are thought to be involved in the initiation of inflammatory diseases. Many bacteria contain, within their genome, genes encoding proteins that share homology to the EGLN family of prolyl hydroxylases. We therefore propose that these bacterial genes may initiate a hypoxic like response at the site of infection thereby causing localized inflammation. The resulting inflammatory infiltrate could then cause the tissue to become hypoxic thereby continuing the cycle of hypoxia response.

In view of the predicted function of the EGLN3 (BAB15101; SEQ ID NO: 85), the EGLN3 splice variant (SEQ ID NO: 85a), EGLN1 (c1orf12; AAG34568; SEQ ID NO: 89), CAB81622, SM20 polypeptides and other equivalent proteins, it is considered likely that this protein will form complexes with various cellular substrates, including Hif1alpha, Hif2alpha, Hif3alpha (see Gu et al., 1998, Gene Expr; 7(3):205-13) and other members of the PAS-domain transcription factor family. A further embodiment of this aspect of the invention thus provides a polypeptide complex comprising a EGLN3 (BAB15101; SEQ ID NO: 85), the EGLN3 splice variant (SEQ ID NO: 85a), EGLN1 (c1orf12; AAG34568; SEQ ID NO: 89), CAB81622, SM20 polypeptide or a functional equivalent thereof, and a polypeptide member of the PAS-domain transcription factor family, such as Hif1alpha, Hif2alpha or Hif3alpha.

These discoveries allow the development of regulators, such as small drug molecules, that affect the activity of these polypeptides, so allowing diseases and physiological conditions that are caused by hypoxia, or in which hypoxia has been implicated, to be treated. These discoveries also allow the development of diagnostic agents that are suitable for the detection of hypoxia in biological tissues and, through the identification of mutations and polymorphisms (such as SNPs) within genes coding for the proteins implicated herein, allows the assessment of an individual's risk of being susceptible to diseases and physiological conditions in which hypoxia is implicated.

As discussed in detail below, fragments and functional equivalents, including splice variants of the EGLN3 (BAB15101; SEQ ID NO: 85), the EGLN3 splice variant (SEQ ID NO: 85a) c1orf12 (AAG34568; SEQ ID NO: 89), CAB81622, SM20 and other equivalent proteins are included within the present invention, in addition to ligands that bind specifically to these proteins. Furthermore, the invention also embraces purified and isolated nucleic acid molecules encoding these proteins, fragments and functional equivalents, vectors containing such nucleic acid molecules and host cells transformed with these vectors.

The therapeutic and diagnostic applications discussed above are also equally relevant to this aspect of the invention. For example, small molecule inhibitors of the EGLN3 (BAB15101; SEQ ID NO: 85), the EGLN3 splice variant (SEQ ID NO: 85a), EGLN1 (c1orf12; AAG34568; SEQ ID NO: 89), CAB81622, SM20 and other equivalent proteins, and encoding genes are envisaged for utility as pharmaceutical agents, particularly in modulating the proline hydroxylase activity of the EGLN3 (BAB15101; SEQ ID NO: 85), the EGLN3 splice variant (SEQ ID NO: 85a), EGLN1 (c1orf12; AAG34568; SEQ ID NO: 89), CAB81622, and SM20 proteins.

Truncated or chimeric inhibitory derivatives of the encoding genes, or distinct genes that encode regulators of the EGLN3 (BAB15101; SEQ ID NO: 85), EGLN3 splice variant (SEQ ID NO: 85a); EGLN1 (c1orf12; AAG34568; SEQ ID NO: 89), CAB81622, and SM20 encoding genes, are also envisaged for utility for gene therapy.

An alignment of the amino acid sequences of rat SM20 (Accession A53770), its human equivalent (Accession BAB15101; SEQ ID NO: 85) and this distinct human homologue (Accession CAB81622 or AAG34568; SEQ ID NO: 89) is shown below:

| | | |
|---|---|---|
| | | 1 50 |
| BAB15101 | (1) | -------------------------------------------------- |
| A53770 | (1) | -------------------------------------------------- |
| AAG34568 | (1) | MANDSGGPGGPSPSERDRQYCELCGKMENLLRCSRCRSSFYCCKEHQRQD |
| Consensus | (1) | |
| | | 51 100 |
| BAB15101 | (1) | -------------------------------------------------- |
| A53770 | (1) | -----MTLRSRRGFLSFLPGLRPPRRWLRISKRGPPTSHWASP-----AL |
| AAG34568 | (51) | WKKHKLVCQGSEGALGHGVGPHQHSGPAPPAAVPPPRAGAREPRKAAARR |
| Consensus | (51) | L G L G A PP A P |
| | | 101 150 |
| BAB15101 | (1) | -------------------------------------------------- |
| A53770 | (41) | GGRTLHYSCRSQSGTPFSSEFQATFPAFAAKVARGPWLPQVVEPPAR--- |
| AAG34568 | (101) | DNASGDAAKGKVKAKPPADPAAAASPCRAAAGGQGSAVAAEAEPGKEEPP |
| Consensus | (101) | S A A P A A P AA A G L EP |
| | | 151 200 |
| BAB15101 | (1) | -----------------------------MPLGHIMRLDLEKIALEYIVP |
| A53770 | (88) | LSASPLCVRSGQALGACTLGVPRLGSVSEMPLGHIMRLDLERIALEYIVP |
| AAG34568 | (151) | ARSSLFQEKANLYPPSNTPGDALSPGGGLRPNGQTKPLPALKLALEYIVP |
| Consensus | (151) | AS KA A T G MPLGHIMRLDLEKIALEYIVP |
| | | 201 250 |
| BAB15101 | (22) | CLHEVGFCYLDNFLGEVVGDCVLERVKQLHCTGALRDGQLAGPRAGVSKR |
| A53770 | (138) | CLHEVGFCYLDNFLGEVVGDCVLERVKQLHYNGALRDGQLAGPRAGVSKR |
| AAG34568 | (201) | CMNKHGICVVDDFLGKETGQQIGDEVRALHDTGKFTDGQLVSQKS-DSSK |
| Consensus | (201) | CLHEVGFCYLDNFLGEVVGDCVLERVKQLH TGALRDGQLAGPRAGVSKR |
| | | 251 300 |
| BAB15101 | (72) | HLRGDQITWIGGNEEGCEAISFLLSLIDRLVLYCGSRLGKYYVKERSKAM |
| AS3770 | (188) | HLRGDQITWIGGNEEGCEAINFLLSLIDRLVLYCGSRLGKYYVKERSKAM |
| AAG34568 | (250) | DIRGDKITWIEGKEPGCETIGLLMSSMDDLIRHCNGKLGSYKINGRTKAM |
| Consensus | (251) | HLRGDQITWIGGNEEGCEAI FLLSLIDRLVLYCGSRLGKYYVKERSKAM |
| | | 301 350 |
| BAB1501 | (122) | VACYPGNGTGYVRHVDNPNGDGRCITCIYYLNKNWDAKLHGGILRIFPEG |
| AS53770 | (238) | VACYPGNGTGYVRHVDNPNGDGRCITCIYYLNKNWDAKLHGGVLRIFPEG |
| AAG34568 | (300) | VACYPGNGTGYVRHVDNPNGDGRCVTCIYYLNKDWDAKVSGGILRIFPEG |
| Consensus | (301) | VACYPGNGTGYVRHVDNPNGDGRCITCIYYLNKNWDAKLHGGILRIFPEG |
| | | 351 400 |
| BAB15101 | (172) | KSFIADVEPIFDRLLFFWSDRRNPHEVQPSYATRYAMTVWYFDAEERAEA |
| AS5770 | (288) | KSFVADVEPIFDRLLFSWSDRRNPHEVQPSYATRYAMTVWYFDAEERAEA |
| AAG34568 | (350) | KAQFADIEPKFDRLLFFWSDRRNPHEVQPAYATRYAITVWYFDADERARA |
| Consensus | (351) | KSFIADVEPIFDRLLFFWSDRRNPHEVQPSYATRYAMTVWYFDAEERAEA |
| | | 401 427 |
| BAB15101 | (222) | KKKFRNLTRKTESALTED--------- |
| A53770 | (338) | KKKFRNLTRKTESALAKD--------- |
| AAG34568 | (400) | KVKYLTGEKGVRVELNKPSDSVGKDVF |
| Consensus | (401) | KKKFRNLTRKTESAL KD |

From this sequence alignment, a highly conserved region of amino acid sequence may be noted (italicized regions), the consensus of which is as follows:

KAMVACYPGNGTGYVRHVDNPNGDGRCITCIYYLNKNWDAKLHGGILRIFPEGKSFIADVEP IFDRLLFFWSDRRNPHEVQPSYATRYAMTVWYFDAEERAEAKKK

This is presumably a functional domain. The region of sequence lost in the splice variant, SEQ ID No. 85a, is directly upstream to the conserved domain. This consensus sequence, and variants thereof, may be used in the identification of other proteins that are implicated in the biological response to hypoxia. This aspect of the invention therefore provides a substantially purified polypeptide comprising the consensus sequence:

KAMVACYPGNGTGYVRHVDNPNGDGRCITCIYYLNKNWDAKLHGGILRIFPEGKSFIADVEP IFDRLLFFWSDRRNPHEVQPSYATRYAMTVWYFDAEERAEAKKK, or a variant thereof.

The invention also provides a substantially purified polypeptide comprising the consensus sequence:

KAMVACYPGNGTGYVRHVDNPNGDGRCITCIYYLNKNWDAKLHGGILRIFPEGKSFIADVEP IFDRLLFFWSDRRNPHEVQPSYATRYAMTVWYFDAEERAEAKKK, or a variant thereof, in the treatment or diagnosis of a hypoxia-related disease or condition.

Neither this consensus domain nor any proteins that contain this domain have been previously associated with the cellular response to hypoxia/ischaemia. Searches of the public databases indicate that the human genome contains several genes that encode proteins that contain this consensus sequence. These proteins may have similar functions or may function in the same biochemical pathway, potentially with an antagonistic effect.

By “variant” is meant a variation of the consensus sequence given above, that exhibits a degree of homology with the consensus sequence above a certain threshold level of identity or similarity. Degrees of identity and similarity can be readily calculated according to methods known in the art (see, for example, Computational Molecular Biology, Lesk, A. M., ed., Oxford University Press, New York, 1988; Biocomputing. Informatics and Genome Projects, Smith, D. W., ed., Academic Press, New York, 1993). Typically, greater than 50% identity between two sequences is considered to be an indication of functional equivalence. Preferably, a variant consensus according to this aspect of the invention exhibits a degree of sequence identity with the consensus sequence given above, of greater than 50%. More preferred polypeptides have degrees of identity of greater than 60%, 70%, 80%, 90%, 95%, 98% or 99%, respectively.

As discussed in detail below, fragments and functional equivalents of these proteins are included within the present invention, in addition to ligands that bind specifically to these proteins. Furthermore, the invention also embraces purified and isolated nucleic acid molecules encoding these proteins, fragments and functional equivalents, vectors containing such nucleic acid molecules and host cells transformed with these vectors. The therapeutic and diagnostic applications discussed above are also equally relevant to this aspect of the invention.

Semaphorin 4b

The polypeptide referred to above as that encoded by SEQ ID No 91 is a specific protein that is termed “Semaphorin 4b”. The gene encoding this protein is regulated (activated) by conditions of hypoxia. The Semaphorin 4b protein is encoded by a gene identified from the EST recited in SEQ ID No 92. The unequivocal and accurate full length cDNA sequence is provided herein as SEQ ID No 92a. The accurate presumptive amino acid sequence is provided herein as SEQ ID No 91. This protein, functionally-equivalent variants of this protein, the encoding nucleic acid molecules and ligands that regulate the activity and/or expression of this gene and protein are claimed above in the context of their role in hypoxia and hypoxia-related disorders.

Semaphorins are a large family of proteins, characterized by the 500 amino acid sema domain (Puschel et al., 1995, Neuron, 14(5): 941-8; Tamagnone and Comoglio, 2000, Trends Cell Biol., 10(9): 377-83). Early work showed a role in the guidance of axons during brain development, and the regulation of cell migration. More recently, specific members of this large family have been associated with cancer (Brambilla et al., Am J Pathol., 2000, 156(3): 939-50), rheumatoid arthritis (Mangasser-Stephan et al., Biochem Biophys Res Commun., 1997, 234(1): 153-6), the immune system (Spriggs, Curr Opin Immunol., 1999, 11(4): 387-91) including B-lymphocyte functions (Hall et al., Proc Natl Acad Sci U S A, 1996, 93(21): 11780-5) and angiogenesis (Miao et al., J Cell Biol., 1999, 146(1): 233-42). This is perhaps not surprising considering that cell migration/trafficking is a key part of inflammation, angiogenesis and tumour metastasis.

There are at least distinct 25 human semaphorin genes and the significance/utility of many of these remains untested. This includes the Semaphorin 4b protein, which is unpublished and until now has not been assigned a full and accurate amino acid sequence.

This aspect of the invention therefore provides a substantially purified semaphorin 4b polypeptide, which polypeptide:

i) comprises the amino acid sequence recited in SEQ ID NO: 91;

ii) has an amino acid sequence encoded by a nucleic acid sequence recited in SEQ ID NO: 92;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

We have made experimental discoveries which link the expression of Semaphorin 4b to factors (hypoxia, gamma IFN and superoxide radicals) that are associated with a variety of human ischaemic and inflammatory diseases. In particular, a key response of cells to hypoxia is to stimulate angiogenesis, and a key part of inflammation is the recruitment and trafficking of immune cells. In light of our discoveries, and what is known about other specific members of the semaphorin family, it is herein proposed that Semaphorin 4b is a regulator of these cellular functions, and thus provides a novel target for therapeutic intervention. This paves the way for the development of therapeutic agents that either potentiate or antagonize functions of Semaphorin 4b. Such agents are likely to be highly valuable in the treatment of human disease.

PI3-kinase Adaptor Proteins

It has also been discovered that the polypeptide whose sequence is recited in SEQ ID NO: 527 is differentially regulated under physiological conditions of hypoxia. This polypeptide is postulated to be active as a PI-3-kinase adapter molecule. This polypeptide is referred to herein as Hu.BCAP-A. The polypeptide sequence recited in SEQ ID NO: 527 was, prior to the present disclosure, totally unknown in the literature and public sequence databases. Accordingly, until now, no biological function has been attributed to this polypeptide sequence. This aspect of the invention thus provides a substantially purified PI-3-kinase adapter polypeptide, which polypeptide:

i) comprises the amino acid sequence recited in SEQ ID NO: 527;

ii) has an amino acid sequence encoded by a nucleic acid sequence recited in SEQ ID NO: 528;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

This gene was identified by screening a cDNA library derived from human peripheral blood mononuclear cells. Two cDNA clones corresponding to BCAP [clone GLB_{—}3 and clone GLB_{—}4] were isolated and Rapid Amplification of cDNA Ends (RACE) was performed on these clones to validate the full length sequence.

We have performed a detailed analysis of this new data, and compared it to a recent prediction of the human BCAP gene made by the human genome Ensembl project (www.ensembl.org/) (Ensembl predicted transcript accession ENST00000286061). The Ensembl gene prediction is equivalent in structure to the mouse sequence (NM_{—}031376), though is truncated at the 5′ end compared to the mouse sequence. The Ensembl gene prediction differs significantly from the Hu.BCAP-A gene and polypeptide sequences presented herein.

By analysing gene expression profiles using RNA prepared from different cell types in conjunction with proprietary nucleic acid arrays, the inventors have found that in hypoxic macrophages, the Hu.BCAP gene is expressed at 6-fold higher levels than the median expression level of this gene throughout other cell types such as adipocytes, cardiomyocytes, endothelial cells, fibroblast cells, hepatocyte cells, mammary epithelial cells, monocyte cells, neuroblastoma cells, renal epithelial cells, and skeletal muscle myocyte cells. These data were substantiated by analysing the same RNA samples using real time quantitative RT-PCR. The advantages of this latter method are that it is more sensitive and because two gene-specific primers are used, the data will be more specific to the gene in question.

Therefore, the levels of the encoded protein in hypoxic monocytes/macrophages, as found at various disease sites, are likely to be higher than in other cell types not involved in the disease process or present at the site of disease. This illuminates a novel utility of this gene as a target for the development of therapeutic products for diseases involving monocytes/macrophages and hypoxia. The clone initially identified by the inventors as corresponding to a gene that is overexpressed in certain tissues in response to hypoxia is IMAGE clone acc:R62339. Database searches for gene sequences showing identity with this IMAGE clone reveal that there are no matching human sequences of any type other than ESTs. This includes full length cDNAs, truncated cDNAs, gene sequences from chromosomal data or hypothetical protein gene sequences. Therefore the human gene represented by IMAGE clone acc:R62339 (Hu.BCAP) was deemed a novel human gene.

By comparison with mouse sequence data (acc AF293806), Hu.BCAP appears likely to encode a novel human Phosphoinositol 3-kinase (PI3-kinase) adapter molecule, homologous to the recently described mouse gene, BCAP. The human gene is neither present on GenBank or published elsewhere. The putative mouse equivalent of this gene has been published and is a known gene sequence named originally by Okada and colleagues as “BCAP” (Okada T, Maeda A, Iwamatsu A, Gotoh K, Kurosaki T. “BCAP: the tyrosine kinase substrate that connects B cell receptor to phosphoinositide 3-kinase activation”. Immunity. December 2000;13(6):817-27) (GenBank accession: NM_{—}031376). However, this and subsequent papers by the same authors indicate that BCAP only has physiological relevance in B-cells.

Protein components of the Phosphoinositol 3-kinase (PI3-kinase) signalling cascade, involved in intracellular signalling, have been implicated clearly as potential drug targets (see Stein RC et al, “PI3-kinase inhibition: a target for drug development” Mol Med Today. September 2000;6(9):347-57). PI3-kinases are a ubiquitously expressed enzyme family and, through the generation of phospholipid second messengers, are key to many cellular processes relevant to human disease, including proliferation, apoptosis and inflammation, motility, carbohydrate metabolism and intracellular protein sorting. These molecules are activated by receptor tyrosine kinases, src-like tyrosine kinases and viral oncoproteins. Studies of mutants that abrogate the binding of PI3-kinases to these molecules has indicated that PI3-kinases mediate mitogenic and cell motility responses of cells to growth factors and oncoproteins. As knowledge of their involvement in disease processes increases, the PI3-kinases and other components of the PI3-kinase signalling cascade appear to be an increasingly attractive target for drug development, particularly in the fields of cancer and other proliferative diseases, and in the treatment of inflammatory and immunological conditions. Evidence of the functional specialization of PI3-kinase isoforms suggests that selective inhibition with acceptable toxicity might be possible. Furthermore, there is evidence from mouse studies that functional ablation of BCAP is likely to have a clinically significant effect on inflammatory disease (Yamazaki et al 2002, J Exp Med 195(5):535-45).

The data presented for the Hu.BCAP gene thus provides evidence that the encoded protein is a novel drug target in humans, specifically targeting monocyte/macrophages at hypoxic disease sites.

In the publication referred to above relating to murine BCAP (Okada et al., 2000, Immunity, (6):817-27), the protein is identified as an adapter molecule connecting the non-receptor protein tyrosine kinase Syk to the p85 subunit of PI3-kinase, and therefore to the pivotal signalling pathways centred around PI3-kinase. The p85 subunit binds activated growth factor receptors and other tyrosine phosphorylated molecules through two Src homology 2 (SH2) domains (Hu et al., (1992) Mol. Cell. Biol. 12:981-990). Although, in Okada's report, Syk is acting as the intracellular signalling component of the B cell antigen receptor, which is present exclusively on B-cells, Syk has been shown to initiate intracellular signalling from other cell surface receptors which are expressed on macrophages, including the Fc gamma receptor, the chemokine receptor CCR5 and macrophage-expressed CD8 (Darby C et al “Stimulation of macrophage Fc gamma RIIIA activates the receptor-associated protein tyrosine kinase Syk and induces phosphorylation of multiple proteins including p95Vav and p62/GAP-associated protein”. J Immunol. 1994 152:5429-37; Kedzierska K et al “FcgammaR-mediated phagocytosis by human macrophages involves Hck, Syk, and Pyk2 and is augmented by GM-CSF.” J Leukoc Biol. August 2001;70(2):322-8; Ganju R K et al “Beta-chemokine receptor CCR5 signals through SHP1, SHP2, and Syk.” J Biol Chem. 2000 275:17263-8; Lin T J et al “Activation of macrophage CD8: pharmacological studies of TNF and IL-1 beta production.” J Immunol. 2000 164:1783-92.). A knock-out mouse has also been generated for BCAP (Yamazaki et al 2002, J Exp Med 195(5):535-45), and it was shown that this mouse knock-out has defective B-cell function.

Alteration of the levels or activity of Hu.BCAP-A and its functional equivalents is thus likely to be of considerable utility in modulating the activity of PI3-kinase. For example, disregulation of the signalling processes involved in cell cycle progression and intracellular protein sorting has detrimental effects, and it is therefore of great importance to understand and control these processes. Of course, this requires identifying the participants in the signalling events involved in these processes and elucidating their mechanism of function. The identification of Hu.BCAP-A and its functional equivalents as such a participant is thus of great importance for a wide range of diagnostic, therapeutic and screening applications. In particular, by knowing the structure of Hu-BCAP-A and its function in the PI3-kinase signalling cascade, it is possible to design compounds which affect this cascade, either by by activating an otherwise inactive pathway, or by inactivating an overly active pathway. Similarly, having identified Hu.BCAP-A as a participant in the PI3-kinase signalling cascade, it is also possible to identify situations in which that cascade is defective, resulting in a particular pathological state.

The novel discovery that Hu.BCAP-A and its functional equivalents are induced by hypoxia allows the development of regulators, such as small drug molecules, that affect the activity of these polypeptides, so allowing diseases and physiological conditions that are caused by hypoxia, or in which hypoxia has been implicated, to be treated. These discoveries also allow the development of diagnostic agents that are suitable for the detection of hypoxia in biological tissues and, through the identification of mutations and polymorphisms (such as SNPs) within genes coding for the proteins implicated herein, allows the assessment of an individual's risk of being susceptible to diseases and physiological conditions in which hypoxia is implicated.

The invention also includes functional equivalents of a PI3-kinase adaptor polypeptide of i), ii) or (iii) as recited above. A functionally-equivalent polypeptide according to this aspect of the invention may be a polypeptide that is homologous to a polypeptide whose sequence is explicitly recited herein. Two polypeptides are said to be “homologous” if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated according to methods known in the art (see, for example, Computational Molecular Biology, Lesk, A. M., ed., Oxford University Press, New York, 1988; Biocomputing. Informatics and Genome Projects, Smith, D. W., ed., Academic Press, New York, 1993).

Typically, greater than 50% identity between two polypeptides is considered to be an indication of functional equivalence, provided that either the biological activity of the polypeptide is retained or the polypeptides possess an antigenic determinant in common. Preferably, a functionally equivalent polypeptide according to this aspect of the invention exhibits a degree of sequence identity with a polypeptide sequence explicitly identified herein, or with a fragment thereof, of greater than 80%. More preferred polypeptides have degrees of identity of greater than 85%, 90%, 95%, 98%, 99% or more, respectively.

Functionally-equivalent polypeptides according to the invention are therefore intended to include natural biological variants (for example, allelic variants or geographical variations within the species from which the polypeptides are derived) and mutants (such as mutants containing amino acid substitutions, insertions or deletions) of the polypeptides whose sequences are explicitly recited herein. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

Preferred functionally-equivalent polypeptides are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. “Mutant” polypeptides also include polypeptides in which one or more of the amino acid residues include a substituent group.

Particularly preferred functionally-equivalents of Hu.BCAP-A are splice variants of the full length Hu. BCAP-A gene. The inventors have identified that a splice variant of Hu.BCAP-A exists, herein termed Hu.BCAP-B. This polypeptide is encoded by an alternative transcript, formed by the splicing pattern of exons: 6, 7, 8, 9, 10, 11, 12, 13, 14, 15, 16, 17, 18, 19. This encodes a protein of 540 amino acids which is a truncated version of BCAP-A. This truncation in the encoded protein is not due to incomplete sequencing, but reflects a different exon usage. Novel functions and utilities are expected for BCAP-B, compared to BCAP-A and mouse BCAP. This splice variant thus forms a preferred functional equivalent of the Hu.BCAP-A polypeptide referred to above.

This aspect of the first embodiment of the invention thus provides a substantially purified PI-3-kinase adapter polypeptide, which polypeptide:

i) comprises the amino acid sequence recited in SEQ ID NO: 529;

ii) has an amino acid sequence encoded by a nucleic acid sequence recited in SEQ ID NO: 530;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

The polypeptide sequence recited in SEQ ID NO: 529 was, prior to the present disclosure, totally unknown in the literature and public sequence databases. Accordingly, until now, no biological function has been attributed to this polypeptide sequence. The inventors have now elucidated a biological function for this polypeptide, in that it has been found to be differentially regulated under physiological conditions of hypoxia. This polypeptide is postulated to be active as a PI-3-kinase adapter molecule. Although the Applicant does not wish to be bound by this theory, since this splice variant is truncated at the N-terminus and therefore lacks the portion of the protein responsible for binding to tyrosine kinases such as Syk, this variant may inhibit PI3-kinase signalling by binding to p85.

The inventors have identified that a further splice variant of Hu.BCAP-A exists, herein termed Hu.BCAP-C. This variant is a further example of a functional equivalent of the Hu.BCAP-A polypeptide referred to above. This variant is formed by the splicing pattern of exons: 1, 2, 3, 4, 7, 8, 9, 10, 11, 12, 13, 14, 15, 16, 17, 18, 19. This encodes a 228aa polypeptide which, as well as being shorter than BCAP-A, also contains novel amino acid sequence derived from exon 3. Furthermore, as a result of exon 4 being directly spliced to exon 7 (rather than to exon 5 as found in BCAP-A) a frameshift is introduced and a small tail of 8 residues is created prior to reaching the first stop codon of this new frame. Agai,n BCAP-C is expected to have novel functions and utilities.

A third aspect of the first embodiment of the invention thus provides a substantially purified PI-3-kinase adapter polypeptide, which polypeptide:

i) comprises the amino acid sequence recited in SEQ ID NO: 531;

ii) has an amino acid sequence encoded by a nucleic acid sequence recited in SEQ ID NO: 532;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii).

The polypeptide sequence recited in SEQ ID NO: 531 was, prior to the present disclosure, totally unknown in the literature and public sequence databases. Accordingly, until now, no biological function has been attributed to this polypeptide sequence. The inventors have now elucidated a biological function for this polypeptide, in that it has been found to be differentially regulated under physiological conditions of hypoxia. This polypeptide is postulated to be active as a PI-3-kinase adapter molecule. Although the Applicant does not wish to be bound by this theory, since this splice variant is truncated at the N-terminus and therefore lacks the portion of the protein responsible for binding to tyrosine kinases such as Syk, this variant may inhibit PI3-kinase signalling by binding to p85.

A simple alignment of the polypeptides encoded by Hu.BCAP-A, Hu.BCAP-B and Hu.BCAP-C reveals the differences indicated below. Residues that differ from Hu.BCAP-A are shown in bold.

| | |
|---|---|
| Hu.BCAP-B | ---------- ---------- ---------- ---------- ---------- |
| Hu.BCAP-A | MAASGVPRGC DILIVYSPDA EEWCQYLQTL FLSSRQVRSQ KILTHRLGPE |
| Hu.BCAP-C | MAASGVPRGC DILIVYSPDA EEWCQYLQTL FLSSRQVRSQ KILTHRLGPE |
| Hu.BCAP-B | ---------- ---------- ---------- ---------- ---------- |
| Hu.BCAP-A | ASFSAEDLSL FLSTRCVVVL LSAELVQHFH KPALLPLLQR AFHPPHRVVR |
| Hu.BCAP-C | ASFSAEDLSL FLSTRCVVVL LSAELVQHFH KPALLPLLQR AFHPPHRVVR |
| Hu.BCAP-B | ---------- ---------- ---------- ---------- ---------- |
| Hu.BCAP-A | LLCGVRDSEE FLDFFPDWAH WQELTCDDEP ETYVAAVKKA ISE------- |
| Hu.BCAP-C | LLCGVRDSEE FLDFFPDWAH WQELTCDDEP ETYVAAVKKA ISEETASHNA |
| Hu.BCAP-B | ---------- ---------- ---------- ---------- ---------- |
| Hu.BCAP-A | ---------- ---------- ----DSGCDS VTDTEPEDEK VVSYSKQQNL |
| Hu.BCAP-C | AQAGLKLLSS SNPPDSASQS TWITDSGCDS VTDTEPEDEK VVSYSKQQNL |
| Hu.BCAP-B | ---------- ---------- ---------- ---------- ---------- |
| Hu.BCAP-A | PTVTSPGNLM VVQPDRIRCG --------AE TTVYVIVRCK LDDRVATEAE |
| Hu.BCAP-C | PTVTSPGNLM VVQPDRIRCG TFHLGTFL-- ---------- ---------- |
| Hu.BCAP-B | ---------- ---------- ---------- ---------- ---------- |
| Hu.BCAP-A | FSPEDSPSVR MEAKVENEYT ISVKAPNLSS GNVSLKIYSG DLVVCETVIS |
| Hu.BCAP-C | ---------- ---------- ---------- ---------- ---------- |
| Hu.BCAP-B | ----MEEIGN LLSNAANPVE FMCQAFKIVP YNTETLDKLL TESLKNNIPA |
| Hu.BCAP-A | YYTDMEEIGN LLSNAANPVE FMCQAFKIVP YNTETLDKLL TESLKNNIPA |
| Hu.BCAP-C | ---------- ---------- ---------- ---------- ---------- |
| Hu.BCAP-B | SGLHLFGINQ LEEEDMMTNQ RDEELPTLLH FAAKYGLKNL TALLLTCPGA |
| Hu.BCAP-A | SGLHLFGINQ LEEEDMMTNQ RDEELPTLLH FAAKYGLKNL TALLLTCPGA |
| Hu.BCAP-C | ---------- ---------- ---------- ---------- ---------- |
| Hu.BCAP-B | LQAYSVANKH GHYPNTIAEK HGFRDLRQFI DEYVETVDML KSHIKEELMH |
| Hu.BCAP-A | LQAYSVANKH GHYPNTIAEK HGFRDLRQFI DEYVETVDML KSHIKEELMH |
| Hu.BCAP-C | ---------- ---------- ---------- ---------- ---------- |
| Hu.BCAP-B | GEEADAVYES MAHLSTDLLM KCSLNPGCDE DLYESMAAFV PAATEDLYVE |
| Hu.BCAP-A | GEEADAVYES MAHLSTDLLM KCSLNPGCDE DLYESMAAFV PAATEDLYVE |
| Hu.BCAP-C | ---------- ---------- ---------- ---------- ---------- |
| Hu.BCAP-B | MLQASTSNPI PGDGFSRATK DSMIRKFLEG NSMGMTNLER DQCHLGQEED |
| Hu.BCAP-A | MLQASTSNPI PGDGFSRATK DSMIRKFLEG NSMGMTNLER DQCHLGQEED |
| Hu.BCAP-C | ---------- ---------- ---------- ---------- ---------- |
| Hu.BCAP-B | VYHTVDDDEA FSVDLASRPP VPVPRPETTA PGAHQLPDNE PYIFKVFAEK |
| Hu.BCAP-A | VYHTVDDDEA FSVDLASRPP VPVPRPETTA PGAHQLPDNE PYIFKVFAEK |
| Hu.BCAP-C | ---------- ---------- ---------- ---------- ---------- |
| Hu.BCAP-B | SQERPGNFYV SSESIRKGPP VRPWRDRPQS SIYDPFAGMK TPGQRQLITL |
| Hu.BCAP-A | SQERPGNFYV SSESIRKGPP VRPWRDRPQS SIYDPFAGMK TPGQRQLITL |
| Hu.BCAP-C | ---------- ---------- ---------- ---------- ---------- |
| Hu.BCAP-B | QEQVKLGIVN VDEAVLHFKE WQLNQKKRSE SFRFQQENLK RLRDSITRRQ |
| Hu.BCAP-A | QEQVKLGIVN VDEAVLHFKE WQLNQKKRSE SFRFQQENLK RLRDSITRRQ |
| Hu.BCAP-C | ---------- ---------- ---------- ---------- ---------- |
| Hu.BCAP-B | REKQKSGKQT DLEITVPIRH SQHLPAKVEF GVYESGPRKS VIPPRTELRR |
| Hu.BCAP-A | REKQKSGKQT DLEITVPIRH SQHLPAKVEF GVYESGPRKS VIPPRTELRR |
| Hu.BCAP-C | ---------- ---------- ---------- ---------- ---------- |
| Hu.BCAP-B | GDWKTDSTSS TASSTSNRSS TRSLLSVSSG MEGDNEDNEV PEVTRSRSPG |
| Hu.BCAP-A | GDWKTDSTSS TASSTSNRSS TRSLLSVSSG MEGDNEDNEV PEVTRSRSPG |
| Hu.BCAP-C | ---------- ---------- ---------- ---------- ---------- |
| Hu.BCAP-B | PPQVDGTPTM SLERPPRVPP RAASQRPPTR ETFHPPPPVP PRGR |
| Hu.BCAP-A | PPQVDGTPTM SLERPPRVPP RAASQRPPTR ETFHPPPPVP PRGR |
| Hu.BCAP-C | ---------- ---------- ---------- ---------- ---------- |

The novel exon “ETASHNAAQAGLKLLSSSNPPDSASQSTWIT” shares homology with the Alu subfamily of repeats, but has no homology with any other protein domain.

Both the Hu.BCAP-A and B variants contain a conserved repeat region that shows homology to the ankyrin repeat (shown italicised). This repeat is thought to be involved in protein-protein interactions. In view of the predicted function of Hu.BCAP-A and its functional equivalents, it is considered likely that this protein will form oligomers with tyrosine kinase proteins such as Syk and/or PI3-kinase proteins, particularly the p85 subunit of a PI3-kinase. A further embodiment of this aspect of the invention thus provides a polypeptide complex comprising a Hu.BCAP-A polypeptide or a functional equivalent thereof, a tyrosine kinase protein, and/or a PI3-kinase protein. Preferred tyrosine kinase proteins include the non-receptor tyrosine kinase Syk and its functional equivalents, and the p85 subunit of a PI3-kinase protein.

Polypeptides of the above aspects of the invention are intended to include fragments of polypeptides according to i) or ii) as defined above, provided that the fragment retains a biological activity that is possessed by the full length polypeptide of i) or ii), or has an antigenic determinant in common with the polypeptide of i) or ii). As used herein, the term “fragment” refers to a polypeptide having an amino acid sequence that is the same as part, but not all, of an amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531, an amino acid sequence that is encoded by a nucleic acid sequence recited in any one of SEQ ID NOS 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, or an amino acid sequence that is encoded by a gene that is linked to a nucleic acid sequence recited in any one of these SEQ ID NOS. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Such fragments may be isolated fragments, that are not part of or fused to other amino acids or polypeptides, or they may be comprised within a larger polypeptide, of which they form a part or region. When comprised within a larger polypeptide, a fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones that express a polypeptide according to the invention or, for example, to purify the polypeptide by affinity chromatography. Such antibodies may also be employed as diagnostic or therapeutic aids, amongst other applications, as will be apparent to the skilled reader.

The term “immunospecific” means that an antibody has substantially greater affinity for a polypeptide according to the invention than their affinity for related polypeptides. As used herein, the term “antibody” is intended to include intact molecules as well as fragments thereof, such as Fab, F(ab′)₂, scFv, including intrabodies, which are capable of binding to the antigenic determinant in question.

The invention also includes functional equivalents of a polypeptide of i), ii) or (iii) as recited above. A functionally-equivalent polypeptide according to this aspect of the, invention may be a polypeptides that is homologous to a polypeptide whose sequence is explicitly recited herein. Two polypeptides are said to be “homologous” if the sequence of one of the polypeptides has a high enough degree of identity or similarity to the sequence of the other polypeptide for the skilled person to determine that they are similar in origin and function. Preferably, homology is used to refer to sequence identity. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated according to methods known in the art (see, for example, Computational Molecular Biology, Lesk, A. M., ed., Oxford University Press, New York, 1988; Biocomputing. Informatics and Genome Projects, Smith, D. W., ed., Academic Press, New York, 1993). Advantageously, the BLAST algorithm is employed, with parameters set to default values. The BLAST algorithm is described in detail at http address www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http address www.ncbi.nih.gov/BLAST/blast_help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994) Nature Genetics 6:119-129.

The five BLAST programs available at http address www.ncbi.nlm.nih.gov perform the following tasks:

blastp compares an amino acid query sequence against a protein sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands).

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see http address www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found bywa filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http address www.ncbi.nlm.nih.gov/BLAST.

Alternatively, sequence homology may be determined by algorithms such as FastA, available at http address biology.ncsa.uiuc.edu/BW30/BW.cgi. FastA is considered to be superior to BLAST for alignment of short sequences. Advantageously, the FastA algorithm is employed using default parameters at http address biology.ncsa.uiuc.edu/BW30/BW.cgi.

Typically, greater than 50% identity between two polypeptides is considered to be an indication of functional equivalence, provided that either the biological activity of the polypeptide is retained or the polypeptides possess an antigenic determinant in common. Preferably, a functionally equivalent polypeptide according to this aspect of the invention exhibits a degree of sequence identity with a polypeptide sequence explicitly identified herein, or with a fragment thereof, of greater than 50%. More preferred polypeptides have degrees of identity of greater than 60%, 70%, 80%, 90%, 95%, 98% or 99%, respectively.

Functionally-equivalent polypeptides according to the invention are therefore intended to include natural biological variants (for example, allelic variants or geographical variations within the species from which the polypeptides are derived) and mutants (such as mutants containing amino acid substitutions, insertions or deletions) of the polypeptides whose sequences are explicitly recited herein. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr.

Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. “Mutant” polypeptides also include polypeptides in which one or more of the amino acid residues include a substituent group.

As discussed above, using a method according to the above-described aspects of the invention it has now been discovered, most surprisingly, that the response to hypoxia differs between different specialized cell types or between different physiological states of the same cell type. For example, it has been found that in macrophage cells, different polypeptides are induced/repressed during different physiological states. Furthermore, it has been found that a subset of this group of polypeptides are regulated only in activated macrophage cells. Macrophages possess various biological activities, including cytotoxic effects towards tumour cells and phagocytosis of bacteria or cellular debris. These form an important and potent arm of innate immunity, and as such must be finely regulated. In the absence of interactions with pathogens or other immune cells, the aforementioned activities of the macrophage are greatly reduced (i.e. resting macrophages). When given appropriate stimuli, such as contact with the lipopolysaccharide surface of bacteria, and/or exposure to T-cell derived interferon gamma, the functional activities of the macrophage are greatly potentiated (i.e. activated macrophage).

The expression of a further subset of these polypeptides has been found herein to be induced in activated macrophages under conditions of hypoxia, whilst a still further subset has been found herein to be repressed in activated macrophages under conditions of hypoxia.

In resting macrophage cells, it has been found that different polypeptides are induced/repressed during the biological response to hypoxia. For example, it has been found that a subset of this group of polypeptides are regulated only in resting macrophage cells. The expression of a further subset of these polypeptides has been found herein to be induced in resting macrophages under conditions of hypoxia, whilst a still further subset has been found herein to be repressed in resting macrophages under conditions of hypoxia.

According to a further aspect of the invention, there is provided a purified and isolated nucleic acid molecule that encodes a polypeptide according to any one of the aspects of the invention discussed above. Such a nucleic acid molecule may consist of the nucleic acid sequence as recited in any one of SEQ ID NOS 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166,168, 170, 172, 174, 176, 178, 180, 182, 184, 186, 188, 190, 192, 194, 196, 198, 200, 202, 204, 206, 208, 210, 212, 214, 216, 528, 530 and 532, or form a redundant equivalent or fragment thereof. This aspect of the invention also includes a purified nucleic acid molecule which hydridizes under high stringency conditions with a nucleic acid molecule as described above.

According to a further aspect of the invention, there is provided an expression vector that contains a purified and isolated nucleic acid molecule according to the aspects of the invention described above. The invention also incorporates a delivery vehicle, such as a liposome, comprising a nucleic acid according to the above-described aspects of the invention.

In a further aspect, the invention provides a host cell transformed with a vector of the above-described aspect of the invention.

In a still further aspect, the invention provides a ligand that binds specifically to a polypeptide according to the above-described aspects of the invention. The ligand may be an antagonist ligand that inhibits the biological activity of the polypeptide, or may be an agonist ligand that activates the hypoxia-induced activity of the polypeptide to augment or potentiate a hypoxia-induced activity.

In a still further aspect of the invention, there is provided a ligand which binds specifically to, and which preferably inhibits the hypoxia-induced activity of, a polypeptide according to any one of the above-described aspects of the invention. Such a ligand may, for example, be an antibody or intrabody that is immunospecific for the polypeptide in question.

According to a further aspect, the invention provides a polypeptide, a nucleic acid molecule, vector, host cell or ligand as described above, for use in therapy or diagnosis of a disease or abnormal physiological condition. Preferably, the disease or abnormal physiological condition that is affected by hypoxia; examples of such diseases include cancer, ischaemic conditions (such as stroke, coronary arterial disease, peripheral arterial disease), reperfusion injury, retinopathy, neonatal stress, preeclapmsia, atherosclerosis, inflammatory conditions (including rheumatoid arthritis), hair loss and wound healing. The undesired celluar process involved in said diseases might include, but is not restricted to; tumorigenesis, angiogenesis, apoptosis, inflammation or erythropoiesis. The undesired biochemical processes involved in said cellular processes might include, but is not restricted to, glycolysis, gluconeogenesis, glucose transportation, catecholamine synthesis, iron transport or nitric oxide synthesis.

According to the invention, a number of known proteins have also been implicated in the biological response to hypoxia. The functions of these proteins are known, meaning that these functions have been annotated in the public databases. The sequences of these proteins are presented in SEQ ID NOS: 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485 and 487.

According to a further aspect of the invention, there is provided a substantially purified polypeptide, which polypeptide:

i) comprises the amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 or 531 or any one of SEQ ID NOS: 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489 and 491;

ii) has an amino acid sequence encoded by a nucleic acid sequence recited in any one of SEQ ID NOS: 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, or encoded by a gene identified from an EST recited in any one of these SEQ ID NOS;

iii) is a fragment of a polypeptide according to i) or ii), provided that said fragment retains a biological activity possessed by the full length polypeptide of i) or ii), or has an antigenic determinant in common with the polypeptide of i) or ii); or

iv) is a functional equivalent of a polypeptide of i), ii) or (iii);

for use in the diagnosis or therapy of tumourigenesis, angiogenesis, apoptosis, the biological response to hypoxia conditions, or a hypoxic-associated pathology.

The invention also provides a purified and isolated nucleic acid molecule that encodes a polypeptide according to this aspect of the invention, for use in the diagnosis or therapy of tumourigenesis, angiogenesis, apoptosis, the biological response to hypoxia conditions, or a hypoxic-associated pathology. The sequences of these molecules are provided in SEQ ID NOS: 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486 and 488. As described above for the EST nucleic acid sequences annotated herein, this aspect of the invention includes redundant equivalents and fragments of the sequences explicitly recited in SEQ ID NOS: 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486 and 488, and purified nucleic acid molecules which hybridize under high stringency conditions with such nucleic acid molecules, and vectors containing such nucleic acid molecules for use in the diagnosis or therapy of tumourigenesis, angiogenesis, apoptosis, the biological response to hypoxia conditions, or a hypoxic-associated pathology.

This aspect of the invention also includes ligands which bind specifically to, and which preferably inhibit the hypoxia-induced activity of, a polypeptide listed in SEQ ID NOS: 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485 and 487, for use in the diagnosis or therapy of tumourigenesis, angiogenesis, apoptosis, the biological response to hypoxia conditions, or a hypoxic-associated pathology.

The invention also provides a pharmaceutical composition suitable for modulating hypoxia and/or ischaemia, comprising a therapeutically-effective amount of a polypeptide, a nucleic acid molecule, vector or ligand as described above, in conjunction with a pharmaceutically-acceptable carrier.

The invention also provides a vaccine composition comprising a polypeptide, or a nucleic acid molecule as described above.

The invention also provides a method of treating a disease in a patient in need of such treatment by administering to a patient a therapeutically effective amount of a polypeptide, a nucleic acid molecule, vector, ligand or pharmaceutical composition as described above. For diseases in which the expression of the natural gene or the activity of the polypeptide is lower in a diseased patient when compared to the level of expression or activity in a healthy patient, the polypeptide, nucleic acid molecule, ligand, compound or composition administered to the patient should be an agonist. For diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist. By the term “agonist” is meant herein, any polypeptide, peptide, synthetic molecule or organic molecule that functions as an activator, by increasing the effective biological activity of a polypeptide, for example, by increasing gene expression or enzymatic activity. By the term “antagonist” is meant herein, any polypeptide, peptide, synthetic molecule or organic molecule that functions as an inhibitor, by decreasing the effective biological activity of the gene product, for example, by inhibiting gene expression of an enzyme or a pharmacological receptor.

The invention also provides for the use of a polypeptide, nucleic acid molecule, vector, ligand or pharmaceutical composition according to any one of the above-described aspects of the invention in modifying the response of a cell to conditions of hypoxia.

The invention also provides a polypeptide, nucleic acid molecule, vector, ligand or pharmaceutical composition according to any one of the above-described aspects of the invention, for use in the manufacture of a medicament for the treatment of a hypoxia-regulated condition.

The invention also provides a method of monitoring the therapeutic treatment of disease or physiological condition in a patient, comprising monitoring over a period of time the level of expression or activity of polypeptide, nucleic acid molecule, vector or ligand in tissue from said patient, wherein altering said level of expression or activity over the period of time towards a control level is indicative of regression of said disease or physiological condition.

The invention also provides a method of providing a hypoxia regulating gene, an apoptotic or an angiogenesis regulating gene by administering directly to a patient in need of such therapy an expressible vector comprising expression control sequences operably linked to one or more of the nucleic acid molecules as described above.

The invention also provides a method of diagnosing a hypoxia-regulated condition in a patient, comprising assessing the level of expression of a natural gene encoding a polypeptide according to any one of the aspects of the invention described above in tissue from said patient and comparing said level of expression or activity to a control level, wherein a level that is different to said control level is indicative of the hypoxia-related condition.

Such a method of diagnosis may be carried out in vitro. One example of a suitable method comprises the steps of: (a) contacting a ligand as described above with a biological sample under conditions suitable for the formation of a ligand-polypeptide complex; and (b) detecting said complex.

A further example of a suitable method may comprises the steps of: a) contacting a sample of tissue from the patient with a nucleic acid probe under stringent conditions that allow the formation of a hybrid complex between a nucleic acid molecule whose sequence is recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486 and 488, and the probe; b) contacting a control sample with said probe under the same conditions used in step a); and c) detecting the presence of hybrid complexes in said samples; wherein detection of levels of the hybrid complex in the patient sample that differ from levels of the hybrid complex in the control sample is indicative of the hypoxia-related condition.

A still further example of a suitable method may comprise the steps of: a) contacting a sample of nucleic acid from tissue of the patient with a nucleic acid primer under stringent conditions that allow the formation of a hybrid complex between a nucleic acid molecule whose sequence is recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486 and 488, and the primer; b) contacting a control sample with said primer under the same conditions used in step a); c) amplifying the sampled nucleic acid; and d) detecting the level of amplified nucleic acid from both patient and control samples; wherein detection of levels of the amplified nucleic acid in the patient sample that differ significantly from levels of the amplified nucleic acid in the control sample is indicative of the hypoxia-related condition.

A still further example of a suitable method may comprise the steps of: a) obtaining a tissue sample from a patient being tested for the hypoxia-related condition; b) isolating a nucleic acid molecule according to any one of the above-described aspects of the invention from said tissue sample; and c) diagnosing the patient for the hypoxia-related condition by detecting the presence of a mutation which is associated with the hypoxia-related condition in the nucleic acid molecule as an indication of the hypoxia-related condition. This method may comprise the additional step of amplifying the nucleic acid molecule to form an amplified product and detecting the presence or absence of a mutation in the amplified product.

Particular hypoxia-related conditions that may be diagnosed in this fashion include cancer, ischaemia, reperfusion, retinopathy, neonatal stress, preeclapmsia, atherosclerosis, rheumatoid arthritis, undesired hair loss, cardiac arrest or stroke, for example, caused by a disorder of the cerebral, coronary or peripheral circulation.

In a further aspect, the invention provides a method for the identification of a compound that is effective in the treatment and/or diagnosis of a hypoxia-regulated condition, comprising contacting a polypeptide, nucleic acid molecule, or ligand according to any one of the above-described aspects of the invention with one or more compounds suspected of possessing binding affinity for said polypeptide, nucleic acid molecule or ligand, and selecting a compound that binds specifically to said nucleic acid molecule, polypeptide or ligand.

For example, the discovery of the involvement of the EGLN3 (BAB15101; SEQ ID NO: 85), the EGLN3 splice variant (SEQ ID NO: 85a), EGLN1 (c1orf12; AAG34568; SEQ ID NO: 89), CAB81622, SM20 and other equivalent proteins, in the hypoxia response allows for the design of screening methods capable of identifying compounds that are effective in the control of the activity of these proteins and which are therefore effective in treating disorders associated with aberrant protein function under conditions of hypoxia.

Such screening methods are preferably designed so as to screen for ligands which bind specifically to, and which preferably inhibit the hypoxia-induced activity of, a EGLN3 (BAB15101; SEQ ID NO: 85), the EGLN3 splice variant (SEQ ID NO: 85a), EGLN1 (c1orf12; AAG34568; SEQ ID NO: 89), CAB81622, or SM20 polypeptide. Such a ligand may, for example, be an antibody that is immunospecific for the polypeptide in question. In an alternative, such a ligand may be a compound that modulates the interaction between the polypeptide or functional equivalent, and its cellular substrate(s). In the case of a EGLN3 (BAB15101; SEQ ID NO: 85), the EGLN3 splice variant (SEQ ID NO: 85a), EGLN1 (c1orf12; AAG34568; SEQ ID NO: 89), CAB81622, or SM20 polypeptide, cellular substrates include Hif1alpha, Hif2alpha, Hif3alpha and other members of the PAS-domain transcription factor family-modulation of the interaction between a EGLN3 (BAB15101; SEQ ID NO: 85), EGLN3 splice variant (SEQ ID NO: 85a), EGLN1 (c1orf12; AAG34568; SEQ ID NO: 89), CAB81622, or SM20 polypeptide and such proteins thus provides a useful mechanism by which to control the cellular response to hypoxia. For the hypoxia-regulated polypeptides implicated herein which are active as prolyl 4-hydroxylases, examples of suitable compounds include substrate-based inhibitors, such as 3-exomethyleneproline peptide like compounds (Tandon M et al (1998) Substrate specificity of human prolyl-4-hydroxylase, Bioorg. Med. Chem. Lett. 8:1139-44), derivatives of proline, derivatives of 4(S)hydroxy proline, and derivatives of 4-keto proline. Further, in view of the fact that the activity of proline-4-hydroxylase is iron, 2-oxoglutarate and asorbic acid dependent (Kivirikko K I, Pihlajaniemi T. (1998) Collagen hydroxylases and the protein disulfide isomerase subunit of prolyl 4-hydroxylases. Adv Enzymol Relat Areas Mol Biol. 72:325-98) and the activity of HIF targeting prolyl hydroxylases such as those recited above is also dependent on these co-factors (Bruick R K, McKnight S L. (2001) A conserved family of prolyl-4-hydroxylases that modify HIF. Science. 294(5545):1337-40), examples of suitable compounds include cofactor-based inhibitors such as 2-oxoglutarate analogues, ascorbic acid analogues and iron chelators.

Methods for measuring prolyl 4-hydroxylase activity, such as mediated by EGLN3 (BAB15101; SEQ ID NO: 85), EGLN3 splice variant (SEQ ID NO: 85a), EGLN1 (c1orf12; AAG34568; SEQ ID NO: 89), CAB81622, or the SM20 polypeptide on a HIF-1α substrate are known to those of skill in the art. For example, enzyme assays using pure or partially pure preparations of enzyme may utilize colourimetric determination of the rate of accumulation of hydroxyproline in the substrate (such as HIF-1α) in the presence and absence of candidate compounds (see Berg, R. A. Methods Enzymol 1982;82 Pt A:372-98. Determination of 3- and 4-hydroxyproline). Enzyme assays for impure preparations of protein, such as cell lysates and membrane preparations should be more specific. For example, antibodies may be developed that are specific for the proline-hydroxylated form of HIF-1α and these can then be utilized in an ELISA technique. Alternatively, if necessary such antibodies could be used on Western blots at a reasonable throughput. Other assays might use mass spectrometry to measure the proportion of proline hydroxylated HIF-1α Most simply, this would involve prior trypsin digestion of the lysate, followed by mass spectrometric analysis of the digest. The peaks would be predicted and identified that correspond to un-hydroxylated and hydroxylated forms of the relevant HIF-1α apolypeptide or peptide.

Cell-based assays may also be used to follow prolyl 4-hydroxylase activity mediated by a polypeptide according to the invention. Such assay could be based on the use of a reporter gene such as firefly luciferase driven by the hypoxia response element (HRE). Functional HIF is required for induction via the HRE, and thus inhibition of HIF prolyl hydroxylase will lead to increased light output. Light is measured in a luminometer, such as is available from Amersham Biosciences and other providers, which typically reads 24, 96 or 384-well plates. One advantage of using cell-based assays is that the enzyme effector is identified under physiological conditions.

In another example, the invention provides a ligand which binds specifically to, and which preferably inhibits the hypoxia-induced activity of, a PI3-kinase polypeptide according to any one of the above-described aspects of the invention. Such a ligand may, for example, be an antibody that is immunospecific for the polypeptide in question. In an alternative, such a ligand may be a compound that modulates the interaction between the Hu.BCAP-A, Hu.BCAP-B or Hu.BCAP-C polypeptide or a functional equivalent thereof, a tyrosine kinase protein (such as srk) and/or a PI3-kinase protein and thereby modulates PI3-kinase activity. Preferably, the interaction between the Hu.BCAP-A polypeptide or a functional equivalent thereof, a tyrosine kinase protein (such as srk) and/or a PI3-kinase protein is inhibited, such that PI3-kinase activity is also inhibited.

Importantly, the identification of the sequence and function Hu.BCAP-A and its functional equivalents allows for the design of screening methods capable of identifying compounds that are effective in the control of PI3-kinase adaptor molecule activity and PI3-kinase activity and which are therefore effective in treating disorders associated with aberrant PI3-kinase function such as cancer and other proliferative diseases, and inflammatory and immunological conditions. Such screening methods are preferably designed so as to screen for ligands which binds specifically to, and which preferably inhibits the hypoxia-induced activity of, a PI3-kinase polypeptide according to any one of the above-described aspects of the invention. Such a ligand may, for example, be an antibody that is immunospecific for the PI3-kinase polypeptide in question. In an alternative, such a ligand may be a compound that modulates the interaction between the Hu.BCAP-A polypeptide or a functional equivalent thereof, a tyrosine kinase protein (such as srk) and/or a PI3-kinase protein and thereby modulates PI3-kinase activity. Preferably, the interaction between the Hu.BCAP-A polypeptide or a functional equivalent thereof, a tyrosine kinase protein (such as srk) and/or a PI3-kinase protein is inhibited, such that PI3-kinase activity is also inhibited.

Assays for PI3-kinase activity are known in the art. For example, different phosphorylated forms of proteins that are substrate for PI3-kinase can be assayed using antibodies that are specific for those forms. For example, the kinase PKB/AKT is activated by two phosphorylation events, both of which are induced by the phosphoinositide dependent kinase PDK1 following PI3-kinase activation. Antibodies that are specific for the various phosphorylated forms of PKB/AKT are available in the art, such as from New England Biolabs (UK) Ltd of Hitchin, Hertfordshire, who supply the following antibodies: catalogue number 9272 can be used to measure total levels of phosphorylated and un-phosphorylated PKB/AKT. Antibody catalogue number 9271 can be used to measure levels of PKB/AKT phosphorylated at Ser473. Antibody catalogue number 9275 can be used to measure levels of PKB/AKT phosphorylated at Thr308. Other suitable antibodies will be known to those of skill in the art. Immunoassays to measure these proteins can be carried out in many different and convenient ways, as is well known to those skilled in the art. For example, for the purpose of screening modulators of BCAP function, enzyme-linked immunoassays can be carried out at high throughput, such as in 96 or 384-well plates, or at higher or lower well density if required by the number of assays to be performed. These assays could be based on the use of NEB 9272 as an unlabelled antibody immobilized in the wells to capture all forms of PKB/AKT, and then fluorescently-labelled or enzyme-linked NEB9271 or NEB9275 could be used to measure Ser473 or Thr308-phosphorylated PKB/AKT, respectively. Alternatively, appropriate labelled antibody could be used to visualize unphosphorylated or phosphorylated forms of PKB/AKT in adherent cells in flat-bottomed wells or discrete regions of a slide that may have received micro-dispensed treatments scanned with a fluorescence microscope.

It is also known that PI3-kinase complex phosphorylates distinct lipids both in vitro and in vivo. In vitro, the PI3-kinase complex can phosphorylate phosphatidylinositol (PI), phosphatidylinositol 4-phosphate (PI4P) and phophatidylinositol 4,5-bisphosphate (PI(4,5)P₂) on the D3 hydroxyl group of the inositol ring, producing phosphatidylinositol 3-phosphate (PI3P), phosphatidylinositol 3,4-bisphosphate (PI(3,4)P₂) and phosphatidylinositol 3,4,5-trisphosphate (PI(3,4,5)P₃) (see Kapellar et al., Bioessays 16:565-576 (1994); and Stephens et al., Biochim. Biophys. Acta 1179:27-75 (1993). Activation of the PI3-kinase complex in cells results in elevated levels of PI(3,4)P₂ and PI(3,4,5)P₃, but not PI3P (see Auger et al., Cell 57:167-175 (1989); Stephens et al., Nature 351:33-39 (1991); Traynor-Kaplan et al., Nature 334:353-356 (1988); and Traynor-Kaplan J. Biol. Chem. 264:15668-15673 (1989)). For determination of the amount of PI3P or PI(3,4)P₂ formed, one may employ any number of a variety of well known assay methods. For example, HPLC analysis can be readily used to quantitatively identify the above described reaction products, using, e.g., tritiated substrates, and the like. Similarly, on a more qualitative level, thin layer chromatography (TLC) can also be used to identify reaction products. The levels of the above described reaction products produced in the presence and absence of the test compound are then compared. Where the presence of the test compound results in an increase or decrease in the level of the reaction product produced by the polypeptide, it is indicative that the test compound is an agonist or antagonist of PI3-kinase activity, respectively, and more particularly, the activity of the Hu.BCAP-A polypeptide and/or its functional equivalents, as described herein.

For example, assay methods specific to PI3-kinase polypeptides may comprise incubating a mixture of PI or PI4P and a polypeptide selected from the group consisting of Hu.BCAP-A or a functional equivalent thereof, PI3-kinase, a tyrosine kinase protein or biologically active fragments thereof, in the presence and absence of the test compound. The mixture is then assayed to determine the amount of PI3P or PI(3,4)P₂ produced in the presence and absence of the test compound. The amount of PI3P or PI(3,4)P₂ produced in the presence of the test compound is compared to the amount of PI3P or PI(3,4)P₂ produced in the absence of the test compound. An increase or decrease in the amount of PI3P or PI(3,4)P₂ in the presence of the test compound is indicative that the test compound is an agonist or antagonist of Hu.BCAP-A-mediated PI3-kinase activity, respectively.

According to a still further aspect of the invention, there is provided a kit useful for diagnosing a hypoxia-regulated condition, comprising a first container containing a nucleic acid probe that hybridizes under stringent conditions with a nucleic acid molecule according to any one of the aspects of the invention described above; a second container containing primers useful for amplifying said nucleic acid molecule; and instructions for using the probe and primers for facilitating the diagnosis of the hypoxia-regulated condition. The kit may additionally comprise a third container holding an agent for digesting unhybridised RNA.

To facilitate in the diagnosis of the hypoxia-regulated condition using one of the methods outlined above, in a further aspect, the invention provides an array of at least two nucleic acid molecules, wherein each of said nucleic acid molecules either corresponds to the sequence of, is complementary to the sequence of, or hybridizes specifically to a nucleic acid molecule according to any one of the aspects of the invention described above. Such an array may contain nucleic acid molecules that either correspond to the sequence of, are complementary to the sequence of, or hybridise specifically to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 90a, 91, 92, 92a, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 215, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295 or more of the nucleic acid molecules implicated in a hypoxia-regulated condition as recited above. The nucleic acid molecules on the array may consist of oligonucleotides of between twelve and fifty nucleotides, more preferably, between forty and fifty nucleotides. Alternatively, the nucleic acid molecules on the array may consist of PCR-amplified cDNA inserts where the nucleic acid molecule is between 300-2000 nucleotides.

In a related aspect, again useful for diagnosis, the invention provides an array of antibodies, comprising at least two different antibody species, wherein each antibody species is immunospecific with a polypeptide implicated in a hypoxia-regulated condition as described above. The invention also provides an array of polypeptides, comprising at least two polypeptide species as recited above, wherein each polypeptide species is implicated in a hypoxia-regulated condition, or is a functional equivalent variant or fragment thereof.

Kits useful in the diagnostic methods of the invention may comprise such nucleic acid, antibody and/or polypeptide arrays.

According to the invention, a kit may also comprise one or more antibodies that bind to a polypeptide as recited above, and a reagent useful for the detection of a binding reaction between said antibody and said polypeptide.

According to a still further aspect of the invention, there is provided a genetically-modified non-human animal that has been transformed to express higher, lower or absent levels of a polypeptide according to any one of the aspects of the invention described above. Preferably, said genetically-modified animal is a transgenic or knockout animal.

The invention also provides a method for screening for a compound effective to treat a hypoxia-regulated condition, by contacting a non-human genetically-modified animal as described above with a candidate compound and determining the effect of the compound on the physiological state of the animal.

General Techniques

The practice of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al., Molecular Cloning, A Laboratory Manual (1989) Cold Harbor-Laboratory Press, Cold Spring Harbor, N.Y. or Ausubel et al., Current protocols in molecular biology (1990) John Wiley and Sons, N.Y.

Unless defined otherwise, all technical and scientific terms used herein have the same meaning as commonly understood by one of ordinary skill in the art to which this invention belongs.

A. Polypeptides

The term “polypeptide” as used herein, refers to a chain (may be branched or unbranched) of two or more amino acids linked to each other by means of a peptide bond or modified peptide bond (isosteres). The term polypeptide encompasses but is not limited to oligopeptides, peptides and proteins. The polypeptide of the invention may additionally be either in a mature protein form or in a pre-, pro- or prepro-protein form that requires subsequent cleavage for formation of the active mature protein. The pre-, pro-, prepro-part of the protein is often a leader or secretory sequence but may also be an additional sequence added to aid protein purification (for example, a His tag) or to conform a higher stability to the protein.

A polypeptide according to the invention may also include modified amino acids, that is, amino acids other than those 20 that are gene-encoded. This modification may be a result of natural processes such as post-translational processing or by chemical modification. Examples of modifications include acetylation, acylation, amidation, ADP-ribosylation, arginylation, attachment of a lipid derivative or phosphatidylinositol, γ-carboxylation, covalent attachment of a flavin or haeme moiety, a nucleotide or nucleotide derivative, cyclisation, demethylation, disulphide bond formation, formation of covalent cross-links, formylation, glycosylation, GPI anchor formation, hydroxylation, iodination, lipid attachment, methylation, myristoylation, oxidation, proteolytic processing, phosphorylation, prenylation, racemisation, selenoylation, sulphation, and ubiquitination. Modification of the polypeptide can occur anywhere within the molecule including the backbone, the amino acid side-chains or at the N- or C-terminals.

A polypeptide according to the invention may either be isolated from natural sources (for example, purified from cell culture), or be a recombinantly produced polypeptide, or a synthetically produced polypeptide or a combination of all the above.

Antibodies

A polypeptide according to the invention, its functional equivalents and/or any immunogenic fragments derived from the polypeptide may be used to generate ligands including immunospecific monoclonal or polyclonal antibodies, or antibody fragments. These antibodies can then be used to isolate or identify clones expressing the polypeptide of the invention or to purify the polypeptide by affinity chromatography. Further uses of these immunospecific antibodies may include, but are not limited to, diagnostic, therapeutic or general assay applications. Examples of assay techniques that employ antibodies are immunoassays, radioimmunoassays (RIA) or enzyme linked immunosorbent assay (ELISA). In these cases, the antibodies may be labelled with an analytically-detectable reagent including radioisotopes, a fluorescent molecule or any reporter molecule.

The term “immunospecific” as used herein refers to antibodies that have a substantially higher affinity for a polypeptide of this invention compared with other polypeptides. The term “antibody” as used herein refers to a molecule that is produced by animals in response to an antigen and has the particular property of interacting specifically with the antigenic determinant that induced its formation. Fragments of the aforementioned molecule such as Fab, F(ab′)₂ and scFv, which are capable of binding the antigen determinant, are also included in the term “antibody”. Antibodies may also be modified to make chimeric antibodies, where non-human variable regions are joined or fused to human constant regions (for example, Liu et al., PNAS, USA, 84, 3439 (1987)). Particularly, antibodies may be modified to make them less immunogenic to an individual in a process such as humanisation (see, for example, Jones et al., Nature, 321, 522 (1986); Verhoeyen et al., Science, 239, 1534 (1988); Kabat et al., J. Immunol., 147, 1709 (1991); Queen et al., PNAS, USA, 86, 10029 (1989); Gorman et al., PNAS, USA, 88, 34181 (1991) and Hodgson et al., Bio/Technology, 9, 421 (1991)). The term “humanised antibody”, as used herein, refers to antibody molecules in which the amino acids of the CDR (complementarity-determining region) and selected other regions in the variable domains of the heavy and/or light chains of a non-human donor antibody have been substituted with the equivalent amino acids of a human antibody. The humanised antibody therefore closely resembles a human antibody, but has the binding ability of the donor antibody. Antibodies may also have a “bispecific” nature, that is, the antibody has two different antigen binding domains, each domain being directed against a different epitope.

Specific polyclonal antibodies may be made by immuno-challenging an animal with a polypeptide of this invention. Common animals used for the production of antibodies include the mouse, rat, chicken, rabbit, goat and horse. The polypeptide used to immuno-challenge the animal may be derived by recombinant DNA technology or may be chemically-synthesised. In addition, the polypeptide may be conjugated to a carrier protein. Commonly used carriers to which the polypeptides may be conjugated include, but are not limited to BSA (bovine serum albumin), thyroglobulin and keyhole limpet haemocyanin. Serum from the immuno-challenged animal is collected and treated according to known procedures, for example, by immunoaffinity chromatography.

Specific monoclonal antibodies can generally be made by methods known to one skilled in the art (see for example, Kohler, G. and Milstein, C., Nature 256, 495-497 (1975); Kozbor et al., Immunology Today 4: 72 (1983); Cole et al., 77-96 in Monoclonal Antibodies and Cancer Therapy, Alan R. Liss, Inc. (1985) and Roitt, I. et al., Immunology, 25.10, Mosby-Year Book Europe Limited (1993)). Panels of monoclonal antibodies produced against the polypeptides of the invention can be screened for various properties, i.e., for isotype, epitope, affinity, etc. against which they are directed. Alternatively, genes encoding the monoclonal antibodies of interest may be isolated from hybridomas, for instance using PCR techniques known in the art, and cloned and expressed in appropriate vectors.

Phage display technology may be utilized to select the genes encoding the antibodies that have exhibited an immunspecific response to the polypeptides of the invention (see McCafferty, J., et al., (1990), Nature 348, 552-554; Marks, J. et al., (1992) Biotechnology 10, 779-783).

Intrabodies are antibody molecules which are expressed within cells in order to modulate the function of specific proteins (Chen et al (1994), Human Gene Therapy vol 5 pp595-601). Intrabodies are included as examples of antibodies within the terms of the present invention. Optimally intrabodies may be in the form of single chain Fv antibodies, or in the form of Fab antibodies. They may be expressed from a wide variety of recombinant vectors, including plasmid or viral-based vectors. The viral vectors may be based on adenovirus, adeno-associated virus, retroviruses or lentiviruses or other systems which are appropriate for non-lytic expression. Intrabodies may be selected directly in yeast using the yeast 2-hybrid system with the target protein as a bait (Auf der Maur et al (2001) FEBS Lett vol 508 pp407-12). This has the advantage that the intrabodies are selected under the reducing conditions which prevail in eukaryotic cells. Alternatively, they may be selected in vitro by phage display or ribosome display or other selection methods based on binding between the intrabody and the target protein or a target region of the protein.

Ligands

The polypeptides of the invention may also be used to search for interacting ligands. Methods for doing this include the screening of a library of compounds (see Coligan et al., Current Protocols in Immunology 1(2); Chapter 5 (1991), isolating the ligands from cells, isolating the ligands from a cell-free preparation or natural product mixtures. Ligands to the polypeptide may activate (agonise) or inhibit (antagonize) its activity. Alternatively, compounds may affect the levels of the polypeptide present in the cell, including affecting gene expression, mRNA stability and the degree of post-translational modification of the encoded protein. The invention thus embraces methods for the identification of a compound that is effective in the treatment and/or diagnosis of disease, comprising contacting a polypeptide, a nucleic acid molecule or host cell according to any one of the embodiments of the invention described herein with one or more compounds suspected of possessing binding affinity for said polypeptide or nucleic acid molecule, and selecting a compound that binds specifically to said nucleic acid molecule or polypeptide, or that affects the level of gene expression, mRNA stability or the degree of post-translational modification of the encoded protein.

Ligands to the polypeptide form a further aspect of the invention, as discussed in more detail above. Preferred “antagonist” ligands include those that bind to the polypeptide of this invention and strongly inhibit any activity of the polypeptide. Preferred “agonist” ligands include those that bind to the polypeptide and strongly induce activity of the polypeptide of this invention or increases substantially the level of the polypeptide in the cell. As defined above, the term “agonist” is meant to include any polypeptide, peptide, synthetic molecule or organic molecule that functions as an activator, by increasing the effective biological activity of a polypeptide, for example, by increasing gene expression or enzymatic activity. The term “antagonist” is meant to include any polypeptide, peptide, synthetic molecule or organic molecule that functions as an inhibitor, by decreasing the effective biological activity of the gene product, for example, by inhibiting gene expression of an enzyme or a pharmacological receptor.

Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

For the hypoxia-regulated polypeptides implicated herein which are active as prolyl 4-hydroxylases, examples of suitable compounds include substrate-based inhibitors, such as 3-exomethyleneproline peptide like compounds (Tandon M et al (1998) Substrate specificity of human prolyl-4-hydroxylase, Bioorg. Med. Chem. Lett. 8:1139-44), derivatives of proline, derivatives of 4(S)hydroxy proline, and derivatives of 4-keto proline. Further, in view of the fact that the activity of proline-4-hydroxylase is iron, 2-oxoglutarate and asorbic acid dependent (Kivirikko KI, Pihlajaniemi T. (1998) Collagen hydroxylases and the protein disulfide isomerase subunit of prolyl 4-hydroxylases. Adv Enzymol Relat Areas Mol Biol. 72:325-98) and the activity of HIF targeting prolyl hydroxylases such as those recited above is also dependent on these co-factors (Bruick R K, McKnight S L. (2001) A conserved family of prolyl-4-hydroxylases that modify HIF. Science. 294(5545): 1337-40), examples of suitable compounds include cofactor-based inhibitors such as 2-oxoglutarate analogues, ascorbic acid analogues and iron chelators.

B. Nucleic Acid Molecules

Preferred nucleic acid molecules of the invention are those which encode the polypeptide sequences recited in any one of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531. Examples of such nucleic acid molecules include those listed in SEQ ID NOS 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, homologous nucleic acids and nucleic acids that are complementary to these nucleic acid molecules. Nucleic acid molecules of this aspect of the invention may be used in numerous methods and applications, as described generally herein. A nucleic acid molecule preferably comprises of at least n consecutive nucleotides from any one of the sequences disclosed in SEQ ID NOS 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, where n is 10 or more. A nucleic acid molecule of the invention also includes sequences that are complementary to the nucleic acid molecule described above (for example, for antisense or probing purposes).

A nucleic acid molecule according to this aspect of the invention may be in the form of RNA, such as mRNA, DNA, such as cDNA, synthetic DNA or genomic DNA. The nucleic acid molecule may be double-stranded or single-stranded. The single-stranded form may be the coding (sense) strand or the non-coding (antisense) strand. A nucleic acid molecule may also comprise an analogue of DNA or RNA, including, but not limited to modifications made to the backbone of the molecule, such as, for example, a peptide nucleic acid (PNA). The term “PNA” as used herein, refers to an antisense molecule that comprises an oligonucleotide of at least five nucleotides in length linked to a peptide backbone of amino acid residues, preferably ending in lysine. The terminal lysine confers solubility to the composition. PNAs may be pegylated to extend their lifespan in a cell, where they preferentially bind complementary single-stranded DNA and RNA and stop transcript elongation (Nielsen, P. E. et al. (1993) Anticancer Drug Des. 8:53-63).

A nucleic acid molecule according to this aspect of the invention can be isolated by cloning, purification or separation of the molecule directly from a particular organism, or from a library, such as a genomic or cDNA library. The molecule may also be synthesised, for example, using chemical synthetic techniques such as solid phase phosphoramidite chemical synthesis. RNA may be synthesized in vitro or in vivo by transcription of the relevant DNA molecule.

Due to the degeneracy of the genetic code, differing nucleic acid sequences may encode the same polypeptide (or mature polypeptide). Thus, nucleic acid molecules included in this aspect of the invention include any molecule comprising a variant of the sequence explicitly recited. Such variants may include variant nucleic acid molecules that code for the same polypeptide (or mature polypeptide) as that explicitly identified, that code for a fragment of the polypeptide, that code for a functional equivalent of the polypeptide or that code for a fragment of the functional equivalent of the polypeptide. Also included in this aspect of the invention, are variant nucleic acid molecules that are derived from nucleotide substitutions, deletions, rearrangements or insertions or multiple combinations of the aforementioned. Such molecules may be naturally occurring variants, such as allelic variants, non-naturally occurring variants such as those created by chemical mutagenesis, or variants isolated from a species, cell or organism type other than the type from which the sequence explicitly identified originated. Variant nucleic acid molecules may differ from the nucleic acid molecule explicitly recited in a coding region, non-coding region or both these regions.

Nucleic acid molecules may also include additional nucleic acid sequence to that explicitly recited, for example, at the 5′ or 3′ end of the molecule. Such additional nucleic acids may encode for a polypeptide with added functionality compared with the original polypeptide whose sequence is explicitly identified herein. An example of this would be an addition of a sequence that is heterologous to the original nucleic acid sequence, to encode a fusion protein. Such a fusion protein may be of use in aiding purification procedures or enabling techniques to be carried out where fusion proteins are required (such as in the yeast two hybrid system). Additional sequences may also include leader or secretory sequences such as those coding for pro-, pre- or prepro-polypeptide sequences. These additional sequences may also include non-coding sequences that are transcribed but not translated including ribosome binding sites and termination signals.

A nucleic acid molecule of the invention may include molecules that are at least 70% identical over their entire length to a nucleic acid molecule as explicitly identified herein in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532. Preferably, a nucleic acid molecule according to this aspect of the invention comprises a region that is at least 80% identical over its entire length to a nucleic acid molecule as explicitly identified herein in these SEQ ID NOS, preferably at least 90%, more preferably at least 95% and most preferably at least 98% or 99% identical. Further preferred embodiments include nucleic acid molecules that encode polypeptides that retain substantially the same biological function or activity as the polypeptide explicitly identified herein. The terms “homology” and “identity” should be given the meanings described in detail above with respect to polypeptide analysis. Preferably, nucleotide homology and identity are assessed using the blastn program available at http address www.ncbi.nlm.nih.gov.

The nucleic acid molecules of the invention can also be engineered using methods generally known in the art. These methods include but are not limited to DNA shuffling; random or non-random fragmentation (by restriction enzymes or shearing methods) and reassembly of fragments; insertions, deletions, substitutions and rearrangements of sequences by site-directed mutagenesis (for example, by PCR). These alterations may be for a number of reasons including for ease of cloning (such as introduction of new restriction sites), altering of glycosylation patterns, changing of codon preferences, splice variants changing the processing, and/or expression of the gene product (the polypeptide) in general or creating fusion proteins (see above).

Hybridization

Nucleic acid molecules of the invention may also include antisense molecules that are partially complementary to a nucleic acid molecule as explicitly identified herein in SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, and which therefore will hybridise to the encoding nucleic acid molecules. These antisense molecules, including oligonucleotides, can be designed to recognise, specifically bind to and prevent transcription of a target nucleic acid encoding a polypeptide of the invention, as will be known by those of ordinary skill in the art (see Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” used herein refers to any process by which a strand of nucleic acid binds with a complementary strand of nucleic acid by hydrogen bonding, typically forming Watson-Crick base pairs. As carried out in vitro, one of the nucleic acid populations is usually immobilized to a surface, whilst the other population is free. The two molecule types are then placed together under conditions conducive to binding.

The phrase “stringency of hybridization” refers to the percentage of complementarity that is needed for duplex formation. “Stringency” thus refers to the conditions in a hybridization reaction that favour the association of very similar molecules over association of molecules that differ. Conditions can therefore exist that allow not only nucleic acid strands with 99-100% complementarity to hybridise, but sequences with lower complementarity (for example, 50%) to also hybridise. High stringency hybridization conditions are defined herein as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridization reaction being carried out at 35° C. (see Sambrook et al. [supra]). Preferably, the conditions used for hybridization are those of high stringency.

Some trans- and cis-acting factors that may affect the binding of two complementary strands include strand length, base composition (GC pairs have an extra hydrogen bond and are thus require more energy to separate than AT pairs) and the chemical environment. The presence of monovalent cations (such as Na⁺) stabilizes duplex formation whereas chemical denaturants such as formamide and urea destabilize the duplex by disruption of the hydrogen bonds. Use of compounds such as polyethylene glycol (PEG) can increase reassociation speeds by increasing overall DNA concentration in aqueous solution by abstracting water molecules. Denhardt's reagent or BLOTTO are chemical agents often added to block non-specific attachment of the liquid phase to the solid support. Increasing the temperature will also increase the stringency of hybridization, as will increasing the stringency of the washing conditions following hybridization (Sambrook et al. [supra]).

Numerous techniques exist for effecting hybridization of nucleic acid molecules. Such techniques usually involve one of the nucleic acid populations being labelled. Labelling methods include, but are not limited to radiolabelling, fluorescence labelling, chemiluminescent or chromogenic labelling or chemically coupling a modified reporter molecule to a nucleotide precursor such as the biotin-streptavidin system. This can be done by oligolabelling, nick-translation, end-labelling or PCR amplification using a labelled polynucleotide. Labelling of RNA molecules can be achieved by cloning the sequences encoding the polypeptide of the invention into a vector specifically for this purpose. Such vectors are known in the art and may be used to synthesise RNA probes in vitro by the addition of an appropriate RNA polymerase such as T7, T3 or SP6 and labelled nucleotides.

Various kits are commercially available that allow the labelling of molecules. Examples include those made by Pharmacia & Upjohn (Kalamazoo, Mich.); Promega (Madison Wis.); and the U.S. Biochemical Corp. (Cleveland, Ohio). Hybridization assays include, but are not limited to dot-blots, Southern blotting, Northern blotting, chromosome in situ hybridization (for example, FISH [fluorescence in situ hybridization]), tissue in situ hybridization, colony blots, plaque lifts, gridded clone hybridization assays, DNA microarrays and oligonucleotide microarrays. These hybridization methods and others, may be used by a skilled artisan to isolate copies of genomic DNA, cDNA, or RNA encoding homologous or orthologous proteins from other species.

The invention therefore also embodies a process for detecting a nucleic acid molecule according to the invention, comprising the steps of: (a) contacting a nucleic probe with a biological sample under hybridising conditions to form duplexes: and (b) detecting any such duplexes that are formed. The term “probe” as used herein refers to a nucleic acid molecule in a hybridization reaction whose molecular identity is known and is designed specifically to identify nucleic acids encoding homologous genes in other species. Usually, the probe population is the labelled population, but this is not always the case, as for example, in a reverse hybridization assay.

One example of a use of a probe is to find nucleic acid molecules with an equivalent function to those that are explicitly identified herein, or to identify additional family members in the same or other species. This can be done by probing libraries,,such as genomic or cDNA libraries, derived from a source of interest, such as a human, a non-human animal, other eukaryote species, a plant, a prokaryotic species or a virus. The probe may be natural or artificially designed using methods recognised in the art (for example, Ausubel et al., [supra]). A nucleic acid probe will preferably possess greater than 15, more preferably greater than 30 and most preferably greater than 50 contiguous bases complementary to a nucleic acid molecule explicitly identified herein.

In many cases, isolated DNA from cDNA libraries will be incomplete in the region encoding the polypeptide, normally at the 5′ end. Methods available for subsequently obtaining full-length cDNA sequence include RACE (rapid amplification of cDNA ends) as described by Frohman et al., (Proc. Natl. Acad. Sci. USA 85, 8998-9002 (1988)), and restriction-site PCR, which uses universal primers to retrieve unknown nucleic acid sequence adjacent to a known locus (Sarkar, G. (1993) PCR Methods Applic., 2:318-322). “Inverse PCR” may also be used to amplify or to extend sequences using divergent primers based on a known region (Triglia, T. et al., (1988) Nucleic Acids Res. 16:8186). Another method which may be used is “capture PCR”, which involves PCR amplification of DNA fragments adjacent to a known sequence in human and yeast artificial chromosome DNA (Lagerstrom, M. et al., (1991) PCR Methods Applic., 1:111-119). Another method which may be used to retrieve unknown sequences is that of Parker, J. D. et al., (1991); Nucleic Acids Res. 19:3055-3060). Additionally, one may use PCR, nested primers, and libraries, such as the PromoterFinder™ library (Clontech, Palo Alto, Calif.) to walk genomic DNA. This latter process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, a nucleic acid molecule according to the invention may be used for chromosome localisation. In this technique, a nucleic acid molecule is specifically targeted to, and can hybridise with, a particular location on an individual human chromosome. The mapping of relevant sequences to chromosomes is an important step in the confirmatory correlation of those sequences with the gene-associated disease. Once a sequence has been mapped to a precise chromosomal location, the physical position of the sequence on the chromosome can be correlated with genetic map data. Such data are found in, for example, McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides-valuable information to investigators searching for disease genes using positional cloning or other gene discovery techniques. Once the disease or syndrome has been crudely localized by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

Nucleic acid molecules of the present invention are also valuable for tissue localisation. Such techniques facilitate the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism, as well as highlighting the involvement of a particular gene in a disease state or abnormal physiological condition.

In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Vectors

The nucleic acid molecules of the present invention may be incorporated into vectors for cloning (for example, pBluescript made by Stratagene) or expression purposes. Vectors containing a nucleic acid molecule explicitly identified herein (or a variant thereof) form another aspect of this invention. The nucleic acid molecule may be inserted into an appropriate vector by any variety of well known techniques such as those described in Sambrook et al. [supra]. Generally, the encoding gene can be placed under the control of a control element such as a promoter, ribosome binding site or operator, so that the DNA sequence encoding the desired polypeptide is transcribed into RNA in the transformed host cell.

Vectors may be derived from various sources including, but not limited to bacterial plasmids, bacteriophage, transposons, yeast episomes, insertion elements, yeast chromosomal elements, viruses for example, baculoviruses and SV40 (simian virus), vaccinia viruses, adenoviruses, fowl pox viruses, pseudorabies viruses, lentiviruses and retroviruses, or combinations thereof, such as those derived from plasmid and bacteriophage genetic elements, including cosmids and phagemids. Human, bacterial and yeast artificial chromosomes (HACs, BACs and YACs respectively) may also be employed to deliver larger fragments of DNA than can be contained and expressed in a plasmid.

Examples of retroviruses include but are not limited to: murine leukaemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), Avian myelocytomatosis virus-29 (MC29), and Avian erythroblastosis virus (AEV). A detailed list of retroviruses may be found in Coffin et al (“Retroviruses” 1997 Cold Spring Harbour Laboratory Press Eds: J M Coffin, S M Hughes, H E Varmus pp 758-763).

Lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et al 1992 EMBO. J 11: 3053-3058; Lewis and Emerman 1994 J. Virol. 68: 510-516). In contrast, other retroviruses—such as MLV—are unable to infect non-dividing cells such as those that make up, for example, muscle, brain, lung and liver tissue.

A vector may be configured as a split-intron vector. A split intron vector is described in PCT patent applications WO 99/15683 and WO 99/15684.

If the features of adenoviruses are combined with the genetic stability of retroviruses/lentiviruses then essentially the adenovirus can be used to transduce target cells to become transient retroviral producer cells that could stably infect neighbouring cells. Such retroviral producer cells engineered to express an antigen of the present invention can be implanted in organisms such as animals or humans for use in the treatment of angiogenesis and/or cancer.

Poxvirus vectors are also suitable for use in accordance with the present invention. Pox viruses are engineered for recombinant gene expression and for the use as recombinant live vaccines. This entails the use of recombinant techniques to introduce nucleic acids encoding foreign antigens into the genome of the pox virus. If the nucleic acid is integrated at a site in the viral DNA which is non-essential for the life cycle of the virus, it is possible for the newly produced recombinant pox virus to be infectious, that is to say to infect foreign cells and thus to express the integrated DNA sequence. The recombinant pox virus prepared in this way can be used as live vaccines for the prophylaxis and/or treatment of pathologic and infectious disease.

For vaccine delivery, preferred vectors are vaccinia virus vectors such as MVA or NYVAC. Most preferred is the vaccinia strain modified virus ankara (MVA) or a strain derived therefrom. Alternatives to vaccinia vectors include avipox vectors such as fowlpox or canarypox known as ALVAC and strains derived therefrom which can infect and express recombinant proteins in human cells but are unable to replicate.

Bacterial vectors may be also used, such as salmonella, listeria and mycobacteria.

Vectors containing the relevant nucleotide sequence may enter the host cell by a variety of methods well known in the art and described in many standard laboratory manuals (such as Sambrook et al., [supra], Ausubel et al., [supra], Davis et al., Basic Methods in Molecular Biology (1986)). Methods include calcium phosphate transfection, cationic lipid-mediated transfection, DEAE-dextran mediated transfection, electroporation, microinjection, scrape loading, transduction, and ballistic introduction or infection.

Host Cells

The choice of host cells is often dependent on the vector type used as a carrier for the nucleic acid molecule of the present invention. Bacteria and other microorganisms are particularly suitable hosts for plasmids, cosmids and expression vectors generally (for example, vectors derived from the pBR322 plasmid), yeast are suitable hosts for yeast expression vectors, insect cell systems are suitable host for virus expression vectors (for example, baculovirus) and plant cells are suitable hosts for vectors such as the cauliflower mosaic virus (CaMV) and tobacco mosaic virus (TMV). Other expression systems include using animal cells (for example, with the LentiVectors™, Oxford BioMedica) as a host cell or even using cell-free translating systems. Some vectors, such as “shuttle vectors” may be maintained in a variety of host cells. An example of such a vector would be pEG 202 and other yeast two-hybrid vectors which can be maintained in both yeast and bacterial cells (see Ausubel et al., [supra] and Gyuris, J., Cell, 75, 791-803).

Examples of suitable bacterial hosts include Streptococci, Staphylococci, Escherichia coli, Streptomyces and Bacillus subtilis cells. Yeast and fungal hosts include Saccharomyces cerevisiae and Aspergillus cells. Mammalian cell hosts include many immortalized cell lines available from the American Type Culture Collection (ATCC) such as CHO (Chinese Hamster Ovary) cells, HeLa cells, BHK (baby hamster kidney) cells, monkey kidney cells, C127, 3T3, BHK, HEK 293, Bowes melanoma and human hepatocellular carcinoma (for example, Hep G2) cells. Insect host cells that are used for baculovirus expression include Drosophila S2 and Spodoptera Sf9 cells. Plant host cells include most plants from which protoplasts be isolated and cultured to give whole regenerated plants. Practically, all plants can be regenerated from cultured cells or tissues, including but not limited to all major species of sugar cane, sugar beet, cotton, fruit and other trees, legumes and vegetables.

Expression Systems

Also included in present invention are expression vectors that comprise a nucleic acid molecule as described above. Expression vectors and host cells are preferably chosen to give long term, high yield production and stable expression of the recombinant polypeptide and its variants.

Expression of a polypeptide can be effected by cloning an encoding nucleic acid molecule into a suitable expression vector and inserting this vector into a suitable host cell. The positioning and orientation of the nucleic acid molecule insert with respect to the regulatory sequences of the vector is important to ensure that the coding sequence is properly transcribed and translated. Alternatively, control and other regulatory sequences may be ligated onto the nucleic acid molecule of this invention prior to its insertion into the expression vector. In both cases, the sequence of the nucleic acid molecule may have to be adjusted in order to effect correct transcription and translation (for example, addition of nucleotides may be necessary to obtain the correct reading frame for translation of the polypeptide from its encoding nucleic acid molecule).

A nucleic acid molecule of the invention may comprise control sequences that encode signal peptides or leader sequences. These sequences may be useful in directing the translated polypeptide to a variety of locations within or outside the host cell, such as to the lumen of the endoplasmic reticulum, to the nucleus, to the periplasmic space, or into the extracellular environment. Such signals may be endogenous to the nucleic acid molecules of the invention, or may be a heterologous sequence. These leader or control sequences may be removed by the host during post-translational processing.

A nucleic acid molecule of the present invention may also comprise one or more regulatory sequences that allow for regulation of the expression of polypeptide relative to the growth of the host cell. Alternatively, these regulatory signals may be due to a heterologous sequence from the vector. Stimuli that these sequences respond to include those of a physical or chemical nature such as the presence or absence of regulatory compounds, changing temperatures or metabolic conditions. Regulatory sequences as described herein, are non-translated regions of sequence such as enhancers, promoters and the 5′ and 3′ untranslated regions of genes. Regulatory sequences interact with host cellular proteins that carry out translation and transcription. These regulatory sequences may vary in strength and specificity. Examples of regulatory sequences include those of constitutive and inducible promoters. In bacterial systems, an example of an inducible promoter is the hybrid lacZ promoter of the Bluescript phagemid (Stratagene, LaJolla, Calif.) or pSport1TM plasmid (Gibco BRL). The baculovirus polyhedrin promoter may be used in insect cells.

An example of a preferred expression system is the lentivirus expression system, for example, as described in International patent application WO98/17815.

Detection of Uptake of Vectors by the Host Organism

Various methods are known in the art to detect the uptake of a nucleic acid or vector molecule by a host cell and/or the subsequent successful expression of the encoded polypeptide (see for example Sambrook et al., [supra]).

Vectors frequently have marker genes that can be easily assayed. Thus, vector uptake by a host cell can be readily detected by testing for the relevant phenotype. Markers include, but are not limited to those coding for antibiotic resistance, herbicide resistance or nutritional requirements. The gene encoding dihydrofolate reductase (DHFR) for example, confers resistance to methotrexate (Wigler, M. et al. (1980) PNAS 77:3567-70) and the gene npt confers resistance to the aminoglycosides neomycin and G-418 (Colbere-Garapin, F. et al (1981) J. Mol. Biol. 150:1-14). Additional selectable genes have been described, examples of which will be clear to those of skill in the art.

Markers however, only indicate that a vector has been taken up by a host cell but does not distinguish between vectors that contain the desired nucleic acid molecule, and those that do not. One method of detecting for the said nucleic acid molecule is to insert the relevant sequence at a position that will disrupt the transcription and translation of a marker gene. These cells can then be identified by the absence of a marker gene phenotype. Alternatively, a marker gene can be placed in tandem with a sequence encoding a polypeptide of the invention under the control of a single promoter. Expression of the marker gene in response to induction or selection usually indicates expression of the tandem gene as well.

More direct and definitive methods to detect the presence of the nucleic acid molecule of the present invention include DNA-DNA or DNA-RNA hybridization with a probe comprising the relevant antisense molecule, as described above. More direct methods to detect polypeptide expression include protein bioassays for example, fluorescence activated cell sorting (FACS), immunoassay techniques such as ELISA or radioimmunoassays.

Alternative methods for detecting or quantitating the presence of the nucleic acid molecule or polypeptide of this invention include membrane, solution or chip-based technologies (see Hampton, R. et al., (1990) Serological Methods, a Laboratory Manual, APS Press, St Paul, Minn.) and Maddox, D. E. et al., (1983) J. Exp. Med, 158, 1211-1216).

Transgenic Animals

In another embodiment of this invention, a nucleic acid molecule according to the invention may be used to create a transgenic animal, most commonly a rodent. The modification of the animal's genome may either be done locally, by modification of somatic cells or by germ line therapy to incorporate inheritable modifications. Such transgenic animals may be particularly useful in the generation of animal models for drug molecules effective as modulators of the polypeptides of the present invention.

Polypeptide Purification

A polypeptide according to the invention may be recovered and purified from recombinant cell cultures by methods including, but not limited to cell lysis techniques, ammonium sulphate precipitation, ethanol precipitation, acid extraction, anion or cation chromatography, phosphocellulose chromatography, hydrophobic interaction chromatography, affinity chromatography, hydroxylapatite chromatography and lectin chromatography, high performance liquid chromatography (HPLC) or fast performance liquid chromatography (FPLC). The polypeptide may need refolding after purification or isolation and many well known techniques are available that will help regenerate an active polypeptide conformation.

Many expression vectors are commercially available that aid purification of the relevant polypeptide. These include vectors that join the sequence encoding the polypeptide to another expressed sequence creating a fused protein that is easier to purify. Ways in which these fused parts can facilitate purification of the polypeptide of this invention include fusions that can increase the solubility of the polypeptide, joining of metal chelating peptides (for example, histidine-tryptophan modules) that allow for purification with immobilized metals, joining of protein A domains which allow for purification with immobilized immunoglobulins and the joining of the domain that is utilized in the FLAGS extension/affinity purification system (Immunex Corp., Seattle, Wash.). Fusion of the polypeptide of this present invention with a secretion signal polypeptide may also aid purification. This is because the medium into which the fused polypeptide has been secreted can subsequently be used to recover and purify the expressed polypeptide.

If necessary, these extraneous polypeptides often comprise a cleavable linker sequence which allows the polypeptide to be isolated from the fusion. Cleavable linker sequences between the purification domain and the polypeptide of the invention include those specific for Factor Xa or for enterokinase (Invitrogen, San Diego, Calif.). One such expression vector provides for expression of a fusion protein containing the polypeptide of the invention fused to several histidine residues preceding a thioredoxin or an enterokinase cleavage site. The histidine residues facilitate purification by IMAC (immobilized metal ion affinity chromatography as described in Porath, J. et al. (1992), Prot. Exp. Purif. 3: 263-281), while the thioredoxin or enterokinase cleavage site provides a means for purifying the polypeptide from the fusion protein. A discussion of vectors that contain fusion proteins is provided in Kroll, D. J. et al. (1993; DNA Cell Biol. 12:441-453).

Assays

Another aspect of this invention includes assays that may be carried out using a polypeptide or nucleic acid molecule according to the invention. Such assays may be for many uses including the development of drug candidates, for diagnostic purposes or for the gathering of information for therapeutics.

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonize) the level of expression of the gene or the activity of the polypeptide of the invention and form a further aspect of the present invention. Examples of suitable compounds are those which are effective to alter the expression of a natural gene which encodes a polypeptide of the invention or to regulate the activity of a polypeptide of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Potential agonists or antagonists include small organic molecules, peptides, polypeptides and antibodies that bind to the polypeptide of the invention and thereby modulate its activity. In this fashion, binding of the polypeptide to normal cellular binding molecules may be potentiated or inhibited, such that the normal biological activity of the polypeptide is enhanced or prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

Alternatively, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

A polypeptide according to the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

A typical polypeptide-based assay might involve contacting the appropriate cell(s) or cell membrane(s) expressing the polypeptide with a test compound. In such assays, a polypeptide according to the invention may be free in solution, affixed to a solid support, borne on a cell surface or located intracellularly. Any response to the test compound, for example a binding response, a stimulation or inhibition of a functional response may then be compared with a control where the cell(s) or cell membrane(s) was/were not contacted with the test compound.

A binding response could be measured by testing for the adherence of a test compound to a surface bearing a polypeptide according to the invention. The test compound may aid polypeptide detection by being labelled, either directly or indirectly. Alternatively, the polypeptide itself may be labelled, for example, with a radioisotope, by chemical modification or as a fusion with a peptide or polypeptide sequence that will facilitate polypeptide detection. Alternatively, a binding response may be measured, for example, by performing a competition assay with a labelled competitor or vice versa. One example of such a technique is a competitive drug screening assay, where neutralising antibodies that are capable of specifically binding to the polypeptide compete with a test compound for binding. In this manner, the antibodies may be used to detect the presence of any test compound that possesses specific binding affinity for the polypeptide. Alternative binding assay methods are well known in the art and include, but are not limited to, cross-linking assays and filter binding assays. The efficacy of binding may be measured using biophysical techniques including surface plasmon resonance and spectroscopy.

High throughput screening is a type of assay which enables a large number of compounds to be searched for any significant binding activity to the polypeptide of interest (see patent application WO84/03564). This is particularly useful in drug screening. In this scenario, many different small test compounds are synthesised on to a solid substrate. The polypeptide is then introduced to this substrate and the whole apparatus washed. The polypeptide is then immobilized by, for example, using non-neutralising antibodies. Bound polypeptide may then be detected using methods that are well known in the art. Purified polypeptide may also be coated directly onto plates for use in the aforementioned drug screening techniques.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signaling and metabolic pathways in which the particular genes/polypeptides are implicated, generate information regarding the identities of polypeptides with which the studied polypeptides interact and provide clues as to methods by which related genes and proteins are regulated.

As described above, such assay methods may preferably be designed so as to screen for ligands which binds specifically to, and which preferably inhibits the hypoxia-induced activity of, a polypeptide according to any one of the above-described aspects of the invention. Such a ligand may, for example, be an antibody that is immunospecific for the polypeptide in question.

In the case of the EGLN3 (BAB15101; SEQ ID NO: 85), the EGLN3 splice variant (SEQ ID NO: 85a), EGLN1 (c1orf12; AAG34568; SEQ ID NO: 89), CAB81622, or SM20 polypeptide, that are herein implicated as hypoxia-responsive prolyl 4-hydroxylases, examples of suitable ligand compounds for screening in the above assays include substrate-based inhibitors, such as 3-exomethyleneproline peptide like compounds (Tandon M et al (1998) Substrate specificity of human prolyl-4-hydroxylase, Bioorg. Med. Chem. Lett. 8:1139-44), derivatives of proline, derivatives of 4(S)hydroxy proline, derivatives of 4-keto proline. Further, in view of the fact that the activity of proline-4-hydroxylase is iron, 2-oxoglutarate and asorbic acid dependent (Kivirikko K I, Pihlajaniemi T. (1998) Collagen hydroxylases and the protein disulfide isomerase subunit of prolyl 4-hydroxylases. Adv Enzymol Relat Areas Mol Biol. 72:325-98) and the activity of HIF targeting prolyl hydroxylases such as those recited above is also dependent on these co-factors (Bruick R K, McKnight S L. (2001) A conserved family of prolyl-4-hydroxylases that modify HIF. Science. 294(5545):1337-40), examples of suitable compounds include cofactor-based inhibitors such as 2-oxoglutarate analogues, ascorbic acid analogues and iron chelators.

In the case of the PI3-kinase polypeptides whose sequences are presented in SEQ ID NOS: 527, 529 and 531, such a ligand may, for example, be a compound that modulates the interaction between the Hu.BCAP-A polypeptide or a functional equivalent thereof, a tyrosine kinase protein and/or a PI3-kinase protein and thereby modulates PI3-kinase activity. Preferably, the interaction between the Hu.BCAP-A polypeptide or a functional equivalent thereof, a tyrosine kinase protein and/or a PI3-kinase protein is inhibited, such that PI3-kinase activity is also inhibited. Assays for PI3-kinase activity are known in the art. For example, different phosphorylated forms of proteins that are substrate for PI3-kinase can be assayed using antibodies that are specific for those forms. For example, the kinase PKB/AKT is activated by two phosphorylation events, both of which are induced by the phosphoinositide dependent kinase PDK1 following PI3-kinase activation. Antibodies that are specific for the various phosphorylated forms of PKB/AKT are available in the art, such as from New England Biolabs (UK) Ltd of Hitchin, Hertfordshire, who supply the following antibodies: catalogue number 9272 can be used to measure total-levels of phosphorylated and un-phosphorylated PKB/AKT. Antibody catalogue number 9271 can be used to measure levels of PKB/AKT phosphorylated at Ser473. Antibody catalogue number 9275 can be used to measure levels of PKB/AKT phosphorylated at Thr308. Other suitable antibodies will be known to those of skill in the art. Immunoassays to measure these proteins can be carried out in many different and convenient ways, as is well known to those skilled in the art. For example, for the purpose of screening modulators of BCAP function, enzyme-linked immunoassays can be carried out at high throughput, such as in 96 or 384-well plates, or at higher or lower well density if required by the number of assays to be performed. These assays could be based on the use of NEB 9272 as an unlabelled antibody immobilized in the wells to capture all forms of PKB/AKT, and then fluorescently-labelled or enzyme-linked NEB9271 or NEEB9275 could be used to measure Ser473 or Thr308-phosphorylated PKB/AKT, respectively. Alternatively, appropriate labelled antibody could be used to visualize unphosphorylated or phosphorylated forms of PKB/AKT in adherent cells in flat-bottomed wells or discrete regions of a slide that may have received micro-dispensed treatments scanned with a fluorescence microscope.

It is also known that PI3-kinase complex phosphorylates distinct lipids both in vitro and in vivo. In vitro, the PI3-kinase complex can phosphorylate phosphatidylinositol (PI), phosphatidylinositol 4-phosphate (PI4P) and phophatidylinositol 4,5-bisphosphate (PI(4,5)P₂) on the D3 hydroxyl group of the inositol ring, producing phosphatidylinositol 3-phosphate (PI3P), phosphatidylinositol 3,4-bisphosphate (PI(3,4)P₂) and phosphatidylinositol 3,4,5-trisphosphate (PI(3,4,5)P₃) (see Kapellar et al., Bioessays 16:565-576 (1994); and Stephens et al., Biochim. Biophys. Acta 1179:27-75 (1993). Activation of the PI3-kinase complex in cells results in elevated levels of PI(3,4)P2 and PI(3,4,5)P₃, but not PI3P (see Auger et al., Cell 57:167-175 (1989); Stephens et al., Nature 351:33-39 (1991); Traynor-Kaplan et al., Nature 334:353-356 (1988); and Traynor-Kaplan J. Biol. Chem. 264:15668-15673 (1989)). For determination of the amount of PI3P or PI(3,4)P2 formed, one may employ any number of a variety of well known assay methods. For example, HPLC analysis can be readily used to identify quantitatively the above described reaction products, using, e.g., tritiated substrates, and the like. Similarly, on a more qualitative level, thin layer chromatography (TLC) can also be used to identify reaction products. The levels of the above described reaction products produced in the presence and absence of the test compound are then compared. Where the presence of the test compound results in an increase or decrease in the level of the reaction product produced by the polypeptide, it is indicative that the test compound is an agonist or antagonist of PI3-kinase activity, respectively, and more particularly, the activity of the Hu.BCAP-A polypeptide and/or its functional equivalents, as described herein.

For example, assay methods may comprise incubating a mixture of PI or PI4P and a polypeptide selected from the group consisting of Hu.BCAP-A or a functional equivalent thereof, PI3-kinase, a tyrosine kinase protein or biologically active fragments thereof, in the presence and absence of the test compound. The mixture is then assayed to determine the amount of PI3P or PI(3,4)P₂ produced in the presence and absence of the test compound. The amount of PI3P or PI(3,4)P₂ produced in the presence of the test compound is compared to the amount of PI3P or PI(3,4)P₂ produced in the absence of the test compound. An increase or decrease in the amount of PI3P or PI(3,4)P₂ in the presence of the test compound is indicative that the test compound is an agonist or antagonist of Hu.BCAP-A-mediated PI3-kinase activity, respectively.

More generally, screening methods according to the invention will concentrate in the early stages on finding candidate compounds, initially by screening libraries of between 30,000 to 50,000 compounds. Once candidates have been identified, subsequent stages involve the verification of the structures of these compounds, for example, using techniques known to those of skill in the art such as High Performance Liquid Chromatography (HPLC)/Mass Spectrometry for dissolved stock and HPLC/Mass Spectrometry and Nuclear magnetic resonance Spectroscopy for solid stock (see Mass Spectrometry in Drug Discovery; Rossi & Sinz (Eds) ISBN: 0824706072; Nuclear Magnetic Resonance Spectroscopy, Nelson). Subsequent follow-up studies will concentrate on evaluating the efficacy of the compounds, generating IC₅₀ values (1-30 μM) from high throughput enzyme inhibition assays (such as using scintillation proximity assays (available from Amersham Pharmacia Biotech) or equivalent techniques). If available, in silico pharmacokinetics studies may be used to expedite the screening process (suitable Bioinformatics expertise may be sought from companies such as Inpharmatica Ltd, London and De Novo Ltd, Cambridge).

Hit to lead optimisation and validation involves evaluating a selection (approximately 200) of the best candidates which yielded ˜1 μM potency (IC50=1 μM) in a high throughput enzyme inhibition assay. Other suitable techniques will involve the determination of the specific absorption rate (SAR) by Caco 2 intestinal cell absorption assay (MultiScreen Caco-2 Assay system available from Millipore; BD BioCoat HTS Caco-2 assay system available from BD Biosicences), the determination of the selectivity of the candidate compounds (against related family members) and evaluating the reversibility and kinetics of binding of these compounds. The studies may also involve the measurement of the in vitro metabolic stability of the candidate compounds in primary human and rat liver microsomes; measurement of the in vitro inhibition and induction of human and mammalian cytochrome P450s; the determination of in vitro toxicity (such as by using MTT assays [available from Roche] and LDH assays [available from Cambridge Biosicence]).

Lead optimisation and validation then takes compounds that satisfy the following criteria for functional assay and in vivo pharmacokinetics studies: 1 nM potency in a high throughput enzyme inhibition assay; solubility of ˜0.1 mg/ml on lead compounds; functionality in the presence of human serum albumin (HSA) and/or rodent serum albumin (RSA); 1000× selectivity against family members; demonstrated in vitro absorption in Caco2 intestinal cells; demonstrated in vitro metabolic stability in primary human and rat liver S9 microsomes; profiles of human and mammalian cytochrome P450 inhibition/induction having been determined (a variety of C¹⁴-labelled substrates are available for P450 assays from Amersham Biosciences); mechanism of interaction with target having been determined; the most advantageous pharmacokinetics specificity profile. This latter test may involve a toxicity test for drugs, such as the Human Ether-a-go-go gene (HERG) potassium channel assay that is a cardiotoxicity test (Compton et al., (1996) Genetically defined therapy of inherited long-QT syndrome. Correction of abnormal repolarization by potassium. Circulation. 94(5): 1018-22), the FLIPR Membrane Potential assay test (kit available from Molecular Devices Ltd) or alternative in vitro mutagenicity tests (such as the Ames test [Benedict et al., (1977) Mutagenicity of cancer chemotherapeutic agents in the Salmonella/microsome test. Cancer Res. 37:2209-13]; kit available from Litron Laboratories] or the clastogenicity assay [Sister Chromatid Exchange assay kit is available from Litron Laboratories]).

Functional (cell-based) assays are then developed to validate the lead compounds. Further studies involve an investigation of in vivo pharmacokinetics data in animal experiments; in vivo positive pharmacokinetics results correlated with in vitro data and in silico data; in vivo activity in a functional animal model; in vivo safety studies—central nervous system (CNS)/cardiovascular (CVS) toxicity profiles should be determined.

Another aspect of this invention provides for any screening kits that are based or developed from any of the aforementioned assays.

C. Pharmaceuticals

A further aspect of the invention provides a pharmaceutical composition suitable for modulating hypoxia and/or ischaemia, comprising a therapeutically-effective amount of a polypeptide, a nucleic acid molecule, vector or ligand as described above, in conjunction with a pharmaceutically-acceptable carrier. A composition containing a polypeptide, nucleic acid molecule, ligand or any other compound of this present invention (herein known as X) is considered to be “substantially free of impurities” (herein known as Y) when X makes up more than 85% mass per mass of the total [X+Y] mass. Preferably X comprises at least 90% of the total X+Y mass. More preferably X comprises at least 95%, 98% and most preferably 99% of the total X+Y mass.

Carriers

Carrier molecules may be genes, polypeptides, antibodies, liposomes or indeed any other agent provided that the carrier does not itself induce toxicity effects or cause the production of antibodies that are harmful to the individual receiving the pharmaceutical composition. Further examples of known carriers include polysaccharides, polylactic acids, polyglycolic acids and inactive virus particles. Carriers may also include pharmaceutically acceptable salts such as mineral acid salts (for example, hydrochlorides, hydrobromides, phosphates, sulphates) or the salts of organic acids (for example, acetates, propionates, malonates, benzoates). Pharmaceutically acceptable carriers may additionally contain liquids such as water, saline, glycerol, ethanol or auxiliary substances such as wetting or emulsifying agents, pH buffering substances and the like. Carriers may enable the pharmaceutical compositions to be formulated into tablets, pills, dragees, capsules, liquids, gels, syrups, slurries, suspensions to aid intake by the patient. A thorough discussion of pharmaceutically acceptable carriers is available in Remington's Pharmaceutical Sciences (Mack Pub. Co., N.J. 1991).

Dosage

The amount of component X in the composition should also be in therapeutically effective amounts. The phrase “therapeutically effective amounts” used herein refers to the amount of agent needed to treat, ameliorate, or prevent (for example, when used as a vaccine) a targeted disease or condition. An effective initial method to determine a “therapeutically effective amount” may be by carrying out cell culture assays (for example, using neoplastic cells) or using animal models (for example, mice, rabbits, dogs or pigs). In addition to determining the appropriate concentration range for X to be therapeutically effective, animal models may also yield other relevant information such as preferable routes of administration that will give maximum effectiveness. Such information may be useful as a basis for patient administration. A “patient” as used in herein refers to the subject who is receiving treatment by administration of X. Preferably, the patient is human, but the term may also include animals.

The therapeutically-effective dosage will generally be dependent on the patient's status at the time of adminstration. Factors that may be taken into consideration when determining dosage include the severity of the disease state in the patient, the general health of the patient, the age, weight, gender, diet, time and frequency of administration, drug combinations, reaction sensitivities and the patient's tolerance or response to the therapy. The precise amount can be determined by routine experimentation but may ultimately lie with the judgement of the clinician. Generally, an effective dose will be from 0.01 mg/kg (mass of drug compared to mass of patient) to 50 mg/kg, preferably 0.05 mg/kg to 10 mg/kg. Compositions may be administered individually to a patient or may be administered in combination with other agents, drugs or hormones.

Routes of Administration

Uptake of a pharmaceutical composition of the invention by a patient may be initiated by a variety of methods including, but not limited to enteral, intra-arterial, intrathecal, intramedullary, intramuscular, intranasal, intraperitoneal, intravaginal, intravenous, intraventricular, oral, rectal (for example, in the form of suppositories), subcutaneous, sublingual, transcutaneous applications (for example, see WO98/20734) or transdermal means.

Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared. Direct delivery of the compositions can generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

Inhibition of Excessive Activity

If a particular disease state is partially or completely caused by an inappropriate excess in the activity of a polypeptide according to the invention, several approaches are available for inhibiting this activity.

One approach comprises administering to a patient an inhibitor compound (antagonist) along with a pharmaceutically acceptable carrier in an amount effective to inhibit the function of the polypeptide, such as by blocking the binding of a ligand, substrate, enzyme, receptor, or by inhibiting a second signal, and thereby alleviating the abnormal condition. Such an antagonist molecule may, for example, be an antibody. Most preferably, such antibodies are chimeric and/or humanised to minimise their immunogenicity, as previously described.

In another approach, soluble forms of the polypeptide that retain binding affinity for the ligand, substrate, enzyme, receptor, in question, may be administered to the patient to compete with the biological activity of the endogenous polypeptide. Typically, the polypeptide may be administered in the form of a fragment that retains a portion that is relevant for the desired biological activity.

In an alternative approach, expression of the gene encoding the polypeptide can be inhibited using expression blocking techniques, such as by using antisense nucleic acid molecules (as described above), either internally generated or separately administered. Modifications of gene expression may be effected by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

Gene silencing approaches may also be undertaken to down-regulate endogenous expression of a gene. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression. This phenomenon is termed post-transcriptional gene silencing (PTGS). Small interfering RNAs (siRNAs) are 21-23 nucleotide dsRNAs that mediate and can induce PTGS in mammalian cells. siRNAs appear to be produced in vivo by cleavage of dsRNA introduced directly or via a transgene or virus. Amplification by an RNA-dependent RNA polymerase (RdRP) may occur in some organisms. siRNAs are incorporated into the RNA-induced silencing complex (RISC), guiding the complex to the homologous endogenous mRNA where the complex cleaves the transcript. The most efficient silencing occurs with siRNA duplexes of 21-mers sense and antisense strands, but paired to have a 2-base 3′ overhang and 19 bases of overlap (see The Max Plank Institute siRNA User Guide; http address www.mpibpc.gwdg.de/index_en.html). The optimum target DNA sequence is AA(N19)TT. The design of such molecules is within the ability of the skilled artisan. In the case of the EGLN3 proteins described herein (SEQ ID NOS: 85 and 85a), for example, the sequence AATCTGCTGGCCTTGTTCATTTT conforms to the above guide, and we recommend this site in the BCAP cDNA sequence for intervention via siRNA. Thus the two single-stranded RNA molecules which would comprise the siRNA agent are: (sense) 5′-UCUGCUGGCCUUGUUCAUUUU and (antisense) 5′-AAUGAACAAGGCCAGCAGAUU. The corresponding siRNA sequence can also be expressed as a hairpin with an appropriate linker from a vector within the cell (see Miyagishi et al, Nature Biotechnol 2002 20:497-500).

In addition, expression of a polypeptide according to the invention may be prevented by using a ribozyme specific to the encoding mRNA sequence for the polypeptide. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

Efficacy of the gene silencing approaches assessed above may be assessed through the measurement of polypeptide expression (for example, by Western blotting), and at the RNA level using TaqMan-based methodologies.

RNA molecules may be modified to increase their intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine that are not as easily recognised by endogenous endonucleases.

Activation of a Polypeptide Activity

If a particular disease state is partially or completely due to a lowered level of biological activity from a polypeptide according to the invention, various methods may be used. An example of such a method includes administering a therapeutically effective amount of compound that can activate (i.e. an agonist) or cause increased expression of the polypeptide concerned. Administration of such a compound may be via any of the methods described previously.

Gene Therapy

Another aspect of the present invention provides for gene therapy methods involving nucleic acid molecules identified herein. Gene therapy may be used to affect the endogenous production of the polypeptide of the present invention by relevant cells in a patient. For example, gene therapy can be used permanently to treat the inappropriate production of a polypeptide by replacing a defective gene with the corrected therapeutic gene.

Treatment may be effected either in vivo or ex vivo. Ex vivo gene therapy generally involves the isolation and purification of the patient's cells, introduction of the therapeutic gene into the cells and finally, the introduction of the genetically-altered cells back into the patient. In vivo gene therapy does not require the isolation and purification of patient cells prior to the introduction of the therapeutic gene into the patient. Instead, the therapeutic gene can be packaged for delivery into the host. Gene delivery vehicles for in vivo gene therapy include, but are not limited to, non-viral vehicles such as liposomes, replication-competent and replication-deficient viruses (for example, adenovirus as described by Berkner, K. L., in Curr. Top. Microbiol. Immunol., 158, 39-66 (1992)) or adeno-associated virus (AAV) vectors as described by Muzyczka, N., in Curr. Top. Microbiol. Immunol., 158, 97-129 (1992) and U.S. Pat. No. 5,252,479. Alternatively, “naked DNA” may be directly injected into the bloodstream or muscle tissue as a form of in vivo gene therapy.

One example of a strategy for gene therapy including a nucleic acid molecule of this present invention may be as follows. A nucleic acid molecule encoding a polypeptide of the invention is engineered for expression in a replication-defective or replication-competent vector, such as a retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a patient for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Genetic delivery of antibodies that bind to polypeptides according to the invention may also be effected, for example, as described in International patent application WO98/55607.

Vaccines

A further embodiment of the present invention provides that the polypeptides .or nucleic acid molecules identified may be used in the development of vaccines. Where the aforementioned polypeptide or nucleic acid molecule is a disease-causing agent, vaccine development can involve the raising of antibodies against such agents. Where the aforementioned polypeptide or nucleic acid molecule is that is upregulated, vaccine development can involve the raising of antibodies or T cells against such agents (as described in WO00/29428).

Vaccines according to the invention may either be prophylactic (i.e. prevents infection) or therapeutic (i.e. treats disease after infection). Such vaccines comprise immunising antigen(s), immunogen(s), polypeptide(s), protein(s) or nucleic acid, usually in combination with pharmaceutically-acceptable carriers as described above. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Vaccination processes may involve the use of heterologous vectors eg: prime with MVA and boost with DNA.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions that may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

The technology referred to as jet injection (see, for example, http address www.powderject.com) may also be useful in the formulation of vaccine compositions.

In accordance with this aspect of the present invention, polypeptides can be delivered by viral or non-viral techniques. Non-viral delivery systems include but are not limited to DNA transfection methods. Here, transfection includes a process using a non-viral vector to deliver a antigen gene to a target mammalian cell. Typical transfection methods include electroporation, nucleic acid biolistics, lipid-mediated transfection, compacted nucleic acid-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1, 2,-bis (oleoyloxy)-3-(trimethylammonio) propane (DOTAP)-cholesterol complexes (Wolff and Trubetskoy 1998 Nature Biotechnology 16: 421) and combinations thereof.

Viral delivery systems include but are not limited to adenovirus vectors, adeno-associated viral (AAV) vectors, herpes viral vectors, influenza, retroviral vectors, lentiviral vectors or baculoviral vectors, venezuelan equine encephalitis virus (VEE), poxviruses such as: canarypox virus (Taylor et al 1995 Vaccine 13:539-549), entomopox virus (Li Y et al 1998 XIIth International Poxvirus Symposium p144. Abstract), penguine pox (Standard et al. J Gen Virol. 1998 79:1637-46) alphavirus, and alphavirus based DNA vectors.

In addition to the use of polypeptide-based vaccines, this aspect of the invention includes the use of genetically-based vaccines, for example, those vaccines that are effective through eliciting the expression of a particular gene (either endogenous or exogenously derived) in a cell, so targeting this cell for destruction by the immune system of the host organism.

A number of suitable methods for vaccination and vaccine delivery systems are described in International patent application WO00/29428.

D. Diagnostics

Another aspect of the present invention provides for the use of a nucleic acid molecule identified herein as a diagnostic reagent.

For example, a nucleic acid molecule may be detected or isolated from a patient's tissue and used for diagnostic purposes. “Tissue” as defined herein refers to blood, urine, any matter obtained from a tissue biopsy or any matter obtained from an autopsy. Genomic DNA from the tissue sample may be used directly for detection of a hypoxia-related condition. Alternatively, the DNA may be amplified using methods such as polymerase chain reaction (PCR), the ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991) and Brunt, J., Bio/Technology, 8, 291-294 (1990)). Such diagnostics are particularly useful for prenatal and even neonatal testing.

A method of diagnosis of disease using a polynucleotide may comprise assessing the level of expression of the natural gene and comparing the level of encoded polypeptide to a control level measured in a normal subject that does not suffer from the disease or physiological condition that is being tested. The diagnosis may comprise the following steps:

a) contacting a sample of tissue from the patient with a nucleic acid probe under stringent conditions that allow the formation of a hybrid complex between a nucleic acid molecule of the invention and the probe;

b) contacting a control sample with said probe under the same conditions used in step a); and

c) detecting the presence of hybrid complexes in said samples;

wherein detection of differing levels of the hybrid complex in the patient sample compared to levels of the hybrid complex in the control sample is indicative of the dysfunction.

A further aspect of the invention comprises a diagnostic method comprising the steps of:

a) obtaining a tissue sample from a patient being tested for disease;

b) isolating a nucleic acid molecule according to the invention from said tissue sample; and

c) diagnosing the patient for disease by detecting the presence of a mutation in the nucleic acid molecule which is associated with disease.

To aid the detection of nucleic acid molecules in the above-described methods, an amplification step, such as PCR, may be included. An example of this includes detection of deletions or insertions indicative of the dysfunction by a change in the size of the amplified product in comparison to the normal genotype. Point mutations can be identified by hybridising amplified DNA to labelled RNA of the invention or alternatively, labelled antisense DNA sequences of the invention. Perfectly matched sequences can be distinguished from mismatched duplexes by RNase digestion or by assessing differences in melting temperatures. The presence or absence of the mutation in the patient may be detected by contacting DNA with a nucleic acid probe that hybridizes to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., PNAS. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. FISH is presently the most commonly applied method and numerous reviews of FISH have appeared (see, for example, Trachuck et a., Science, 250, 559-562 (1990), and Trask et aL., Trends, Genet., 7, 149-154 (1991)).

Arrays

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M.Chee et al., Science (1996), Vol 274, pp 610-613).

In one embodiment, the array is prepared and used according to the methods described in WO95/11995 (Chee et al); Lockhart, D. J. et al. (1996) Nat. Biotech. 14: 1675-1680); and Schena, M. et al. (1996) PNAS 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application W095/251116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

Diagnostics Using Polypeptides or mRNA

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). One example of this aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterized by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilize the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

Diagnostic Kits

A diagnostic kit of the present invention may comprise:

(a) a nucleic acid molecule of the present invention;

(b) a polypeptide of the present invention; or

(c) a ligand of the present invention.

In reply to: one aspect of the invention, a diagnostic kit may comprise a first container containing a nucleic acid probe that hybridizes under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, an array of antibody molecules, and/or an array of polypeptide molecules, as discussed in more detail above.

Such kits will be of use in diagnosing a disease or susceptibility to disease, particularly inflammation, oncology, or cardiovascular disease.

Various aspects and embodiments of the present invention will now be described in more detail by way of example, with particular reference to polypeptides regulated differentially under hypoxic conditions as opposed to normoxic conditions. It will) be appreciated that modification of detail may be made without departing from the scope of the invention.

### BRIEF DESCRIPTION OF THE DRAWINGS

FIG. 1 shows a scatter plot, showing normalized signal intensities in hypoxia versus normoxia, with each dot representing a single gene.

FIG. 2: Hypoxia responses amplified by HIF1alpha overexpression. Data shown is the average of 4 repeat experiments. Values represent fold change as compared to untreated cells (condition 1). Error bars represent standard error of the mean.

FIG. 3: Hypoxia responses amplified by EPAS1 overexpression. Data shown is the average of 4 repeat experiments. Values represent fold change as compared to untreated cells (condition 1). Error bars represent standard error of the mean.

FIG. 4: Hypoxia responses amplified by HIF1alpha/EPAS1 overexpression. Data shown is the average of 4 repeat experiments. Values represent fold change as compared to untreated cells (condition 1). Error bars represent standard error of the mean.

FIG. 5 shows genes that are induced by hypoxia to a greater degree in resting macrophages, as compared to activated macrophages. Error bars show the standard deviation from both repeat experiments and multiple exposures from single experiments. These data are not shown in table form. All bars are ratios of mRNA expression in hypoxia/normoxia. These are calculated separately for resting (light bars) and activated (dark bars) macrophages, and do not illustrate differences resulting from activation in nonnoxia.

FIG. 6 shows genes which are induced by hypoxia to a greater degree in activated macrophages, compared to resting macrophages.

FIG. 7 shows genes that are repressed by hypoxia to a greater degree in activated macrophages.

For FIGS. 8, 9a, 9c, 10-32a, 32d and 33-52, mRNA levels, determined from a custom gene array, of particular genes are shown on the Y-axis, expressed as a value as compared to the median expression level of this gene throughout all samples. Eleven primary human cell types as shown on the x-axis were cultured in normoxia (black), or exposed to hyopxia for 6 hr (grey) or 18 hr (white).

FIG. 8: Ecotropic viral integration site 2A (SEQ ID NO::475/476).

FIG. 9a: Novel PI-3-kinase adapter (SEQ ID NO::79/80); Image clone accession R62339.

FIG. 9b: TaqMan Real-time Q-RT-PCR data for Novel PI-3-kinase adapter (SEQ ID NO::79/80); Image clone accession R62339.

FIG. 9c: IMAGE clone acc R59598 (Syk).

FIG. 10: Regulator of G-protein signalling 1 (SEQ ID NO::375/376)

FIG. 11: GM2 ganglioside activator protein (SEQ ID NO::389/390)

FIG. 12: hypothetical protein PRO0823 (SEQ ID NO::21/22)

FIG. 13: CYP1 (cytochrome P450, subfamily XXVIIB) (SEQ ID NO::339/340)

FIG. 14: Alpha-2-macroglobulin (SEQ ID NO::405/406)

FIG. 15: Interleukin 1 receptor antagonist (SEQ ID NO::357/358)

FIG. 16: SCYA3L (SEQ ID NO::469/470)

FIG. 17: CFFM4 (SEQ ID NO::433/434)

FIG. 18: Pleckstrin (SEQ ID NO::431/432)

FIG. 19: CYP1B1 (SeqID:325/326)

FIG. 20: CYP1B1 (SeqID:137/138)

FIG. 21: hypothetical protein FLJ13511 (SeqID:163/164)

FIG. 22: Hematopoietic Zinc finger protein (SeqID:17/18)

FIG. 23: Osteopontin (SeqID:267/268)

FIG. 24: Osteopontin (SeqID:267/268)

FIG. 25: Adipophilin (SeqID:313/314)

FIG. 26: Adipophilin (SeqID:313/314)

FIG. 27: Adipophilin (SeqID:313/314)

FIG. 28: Adipophilin (SeqID:313/314)

FIG. 29: hypothetical protein FLJ22690 (SeqID:205/206)

FIG. 30: cDNA DKFZp586E1624 (SeqID: 65/66)

FIG. 31: EST (SeqID:197/198)

FIG. 32a: EGL nine (C.elegans) homolog 3 (SeqID:85/86)

FIG. 32b: Gene expression profiles in macrophages with and without activation. mRNA levels, determined from a custom gene array, of c1orf12 are shown on the Y-axis, expressed as a value compared to the mean value of a set of control genes on each array (per-chip normalisation). All cells were human macrophages, cultured either without cytokines or with IL-10 or with the combination of IFN□ and LPS in normoxia and hypoxia.

FIG. 32c: Gene expression profiles in macrophages with and without activation. mRNA levels, determined from a custom gene array, of EGLN3 are shown on the Y-axis, expressed as a value compared to the mean value of a set of control genes on each array (per-chip normalisation). All cells were human macrophages, cultured either without cytokines or with IL-10 or with the combination of IFN□ and LPS in normoxia and hypoxia.

FIG. 32d: C1orf12 (SeqID: 89.90)

FIG. 32e: The effect of EPAS/HIF overexpression on expression of the gene C1orf12 EGLN genes using a custom gene array. mRNA expression levels of the gene c1ORF12 as determined by the custom array, in response to hypoxia and adenoviral over-expression of HIF or EPAS are shown. Experimental conditions are as follows: #1 no adeno/normoxia; #2 empty adeno (low dose)/normoxia; #3 empty adeno (high dose)/normoxia; #4 empty adeno (low dose)/hypoxia; #5 empty adeno (high dose)/hypoxia; #6 HIF-1 adeno (low dose)/hypoxia; #7 HIF-1 adeno (high dose)/hypoxia; #8 EPAS adeno (low dose)/hypoxia; #9 EPAS adeno (high dose)/hypoxia. Error bars are the standard error of the mean.

FIG. 32f: The effect of EPAS/HIF overexpression on expression of the gene EGLN3 gene using a custom gene array. mRNA expression levels of the gene EGLN3 as determined by the custom array, in response to hypoxia and adenoviral over-expression of HIF or EPAS are shown. Experimental conditions are as follows: #1 no adeno/normoxia; #2 empty adeno (low dose)/normoxia; #3 empty adeno (high dose)/normoxia; #4 empty adeno (low dose)/hypoxia; #5 empty adeno (high dose)/hypoxia; #6 HIF-1 adeno (low dose)/hypoxia; #7 HIF-1 adeno (high dose)/hypoxia; #8 EPAS adeno (low dose)/hypoxia; #9 EPAS adeno (high dose)/hypoxia. Error bars are the standard error of the mean.

FIG. 32g: The effect of EPAS/HIF overexpression on expression of the EGLN3 gene using AffyMetrix Hu95 ver2 GeneChips. mRNA expression levels of the gene in response to hypoxia and adenoviral over-expression of HIF or EPAS are shown. Graphs show the mean of two replicate arrays, with error bars as standard deviation. Above each graph, data values are shown, including the normalized values and raw values (the AffyMetrix average difference parameter) and Present/Absent flags.

FIG. 32h: The effect of EPAS/HIF overexpression on expression of the c1orf12 gene using AffyMetrix Hu95 ver2 GeneChips. mRNA expression levels of the gene in response to hypoxia and adenoviral over-expression of HIF or EPAS are shown. Graphs show the mean of two replicate arrays, with error bars as standard deviation. Above each graph, data values are shown, including the normalized values and raw values (the AffyMetrix average difference parameter) and Present/Absent flags.

FIG. 32i: Flag immunocytochemistry in HEK293T cells

FIG. 32j: Human Cardiomyocyte Caspase Activity after 72 hours transduction with EIAV-ELG9-Homolog 3

FIG. 32k: Qualitiative RT PCR of EGLN3 isoforms in various primary cell types. Cell types are as follows: “Adipocytes” (Clonetics CC-2568; derived from subcutaneous adult adipose tissue), “Cardiomyocyte” (Clonetics CC-2582; derived from fetal tissue; prior to experimentation cultured in minimal medium: DMEM, 4% Horse serum), “HUVEC” (TCS CellWorks ZHC-2101 human umbilical vein endothelial cells), “Dermal fibroblast” (Clonetics CC-2511 dermal fibroblasts derived from adult tissue), “Macrophage” (derived from human blood as described elsewhere in the specification), “Mammary epithelium” (Clonetics CC-2551; derived from adult tissue), “Monocyte” (derived from human blood as described elsewhere in the specification but without the 7 day differentiation culture period), “SHSY5Y” (neuroblastoma-derived cell line SH-SY5Y), “Renal epithelial” (Clonetics CC-2556; derived from fetal tissue), “SKM” skeletal muscle myocyte (Clonetics CC-2561; derived from adult tissue).

“N”=cells growing in normoxia. “6 hr H”=after exposure to 0.1% oxygen for 6 hr. “18 hr H”=after exposure to 0.1% oxygen for 18 hr. “+”=positive control RNA. “−”=no RNA negative control.

FIG. 32l: Effects of prolyl hydroxylase inhibitors on EGLN3 and the EGLN3 splice variant. Abbreviations are as follows: DAU=daunorubicin; MIMO=L-mimosine; HM=HREluc, MIMO; HH=HIF,HREluc; S1H=SVFL1,HREluc; S1HM=S1H, MIMO; S2H=SVFL2,HREluc; S2HM=S2H, MIMO; S1HH=SVFL1,HIF,HREluc; S1HHM=S1HH, MIMO; S2HH=SVFL2,HIF,HREluc; S2HHM=S2HH, MIMO.

FIG. 33: Novel Metallothionein (SeqID:83/84)

FIG. 34: hypothetical protein hqp0376 (SeqID:337/338)

FIG. 35: Metallothionein 2A (SeqID:265/266)

FIG. 36: Metallothionein 1G (SeqID:243/244)

FIG. 37: Metallothionein 1H (SeqID: 239/240)

FIG. 38: Hepcidin antimicrobial peptide (SeqID:141/142)

FIG. 39: EST (SeqID: 117/118)

FIG. 40: hypothetical protein FLJ22622 (SeqID:129/130)

FIG. 41: TRIP-Br2 (SeqID:31/32)

FIG. 42: Tumor protein D52 (SeqID:301/302)

FIG. 43: Semaphorin 4b (SeqID:91/92/92a)

FIG. 44: Dec-1 (SeqID:371/372)

FIG. 45: Calgranulin A (SeqID:447/448)

FIG. 46: ERO1 (S. cerevisiae)-like (SeqID:67/68)

FIG. 47: hypothetical protein FLJ20500 (SeqID:25/26)

FIG. 48: N-myc downstream regulated (SeqID:229/230)

FIG. 49: Decidual protein induced by progesterone (SeqID:387/388)

FIG. 50: Integrin, alpha 5 (SeqID:379/380)

FIG. 51: Tissue factor (SeqID:225/226)

FIG. 52: COX-2 (SeqID:237/238)

FIG. 53: Genes up-regulated by macrophage activation. Normalized mRNA levels in the 6 experimental conditions (#1 no cytokines/normoxia, #2 no cytokines/hypoxia, #3 IL-10/normoxia, #4 IL-10/hypoxia, #5 LPS/IFN/normoxia, #6 LPS/IFN/hypoxia) are shown as values referenced to the median value of that gene throughout all 6 experimental conditions. Error bars show the standard error of the mean.

FIG. 54: Genes downregulated by macrophage activation (I)

FIG. 55: Genes downregulated by macrophage activation (II)

FIG. 56: Genes downregulated by macrophage activation (III)

FIG. 57 shows an RNase protection assay for the gene encoding Semaphorin 4b.

FIG. 58 shows a Northern blot showing the size of the mRNA and tissue distribution for the Semaphorin 4b gene.

### DETAILED DESCRIPTION OF THE INVENTION

### EXAMPLES

Summary

Subtracted cDNA libraries were separately prepared for hypoxic macrophages and cardiomyoblasts. This involved harvesting RNA from cells both in normoxia and hypoxia, and preparing cDNA. Subtractive hybridization/suppression PCR was then performed to remove genes from the hypoxic cell cDNA, which are also present in cDNA from normoxic cells. Insert DNA from the libraries was PCR amplified and arrayed onto duplicate membranes. Quantitative hybridizations with pre-library cDNA material (normoxia and hypoxia) were done to identify clones in the libraries that actually contain hypoxia inducible genes. The insert DNA was then sequenced.

This procedure was done independently for macrophage and cardiomyoblast. The hypoxia inducible genes identified from these different cell types differed widely, with only a minority of these genes being identified from both cell types.

To characterize the differences between the two tissues further, arrays were produced containing all confirmed hypoxia-inducible genes from the macrophage library. Replicate arrays were hybridised with cDNA from normoxic and hypoxic cardiomyoblasts to allow quantitative evaluation of these genes in the cardiomyoblast. This revealed quantitative differences in the hypoxia induced activation these genes in the two cell types.

Example 1a

Comparison of the hypoxic-response between human macrophages and cardiomyoblasts by a subtraction cloning/array screening approach

Methods/Results

To isolate human macrophage, monocytes were derived from peripheral blood of healthy human donors. 100 ml bags of buffy coat from the Bristol Blood Transfusion Centre were mixed with an equal volume of RPMI1640 medium (Sigma). This was layered on top of 10 ml ficol-paque (Pharmacia) in 50 ml centrifuge tubes and centrifuged for 25 min at 800×g. The interphase layer was removed, washed in MACS buffer (phosphate buffered saline pH 7.2, 0.5% bovine serum albumin, 2 mM EDTA) and resuspended at 80 microliter per 10n7 cells. To this 20 microliter CD14 Microbeads (Miltenyi Biotec) were added, and the tube incubated at 4 degrees for 15 min. Following this one wash was performed in MACS buffer at 400×g and the cells were resuspended in 3 ml MACS buffer and separated on an LS+MACS Separation Column (Miltenyi Biotec) positioned on a midi-MACS magnet (Miltenyi Biotec). The column was washed with 3×3 ml MACS buffer. The column was removed from the magnet and cells were eluted in 5 ml MACS buffer using a syringe. Cells were washed in culture medium (AIM V (Sigma) supplemented with 2% human AB serum (Sigma), and resuspended at 2×10n5 cells per ml in the same medium and placed in large teflon-coated culture bags (Sud-Laborbedarf GmbH, 82131 Gauting, Germany) and transferred to a tissue culture incubator (37 degrees, 5% CO2) for 7-10 days. During this period monocytes spontaneously differentiate to macrophages. This is confirmed by examining cell morphology using phase contrast microscopy. Cells are removed from the bags by placing at 4 degrees for 30 min and emptying the contents. The cells are then washed and resuspended in culture medium at 5×105 cell/ml and plated out in Primeria 10 cm tissue culture petri dishes (Falcon Becton Dickinson) at 5×10n6 cells per dish. Culture is continued for 16-24 hr to allow cell adherence, prior to experimentation involving hypoxia.

As an alternative primary cell type human cardiomyoblast cultures were established. Cells derived from the ventricular tissue of newborn or foetal hearts were purchased from BioWhittaker (CC-2582). Growth conditions were used to allow maximum expansion of the cells in vitro, by using a medium rich in growth factors. Under such conditions cardiomyoblast-like cells predominate (the developmental precursor of cardiomyocytes). This has been previously described by Goldman and Wurzel (In Vitro Cell. Dev. Biol. 28A: 109-119 (1992)) and Goldman et al., (1996, Exp.Cell.Res. 228(2): 237-245).

For these cultures, cells were seeded at 1×10⁶ per T150 flask in human smooth muscle growth medium (TCS CellWorks ZHM-3935) and were expanded in the same medium up to a maximum number of 4 passages. The growth medium is purchased pre-prepared, and includes in the formula, 5% fetal bovine serum, insulin, epidermal growth factor and fibroblast growth factor. Prior to experimentation involving hypoxia, cells were plated onto 10 cm tissue culture petri dishes and allowed to reach confluency.

For experimentation with hypoxia, for all cell types, an equal number of identical culture dishes were divided into two separate incubators: One at 37 degrees, 5% CO2, 95% air (=Normoxia) and the other at 37 degrees, 5% CO₂, 94.9% Nitrogen, 0.1 % Oxygen (=Hypoxia). After 6 hours culture under these conditions, the dishes were removed from the incubator, placed on a chilled platform, washed in cold PBS and total RNA was extracted using RNazol B (Tel-Test, Inc; distributed by Biogenesis Ltd) following the manufacturer's instructions. Polyadenylated mRNA was extracted from the total RNA using a commercial kit following the manufacturer's instructions (Promega; PolyATract mRNA isolation System IV).

The hypoxia period of 6 hr was previously determined to be sufficient to allow the induction of known hypoxia-regulated genes, as determined by RNase protection assays. During these preliminary studies it was noted that macrophages, cardiomyoblasts and an additional control cell type, Jurkat T-cells, showed different patterns of gene induction in response to hypoxia:

| | level of hypoxia-induced | | |
|---|---|---|---|
| | increase in mRNA levels | | |
| Known Hypoxia-inducible gene | Macrophage | Myoblast | T-cell |
| phosphoglycerate kinase-1 | none | none | high |
| (PGK) | | | |
| vascular endothelial growth factor-A | high | low | high |
| (VEGF) | | | |
| solute carrier family 2, member 1 | high | low | high |
| (Glut-1) | | | |

Separate subtracted cDNA populations were generated from mRNA extracted from hypoxic macrophages and hypoxic cardiomyoblasts, using a combination of two kits, purchased from Clontech Laboratories—SMART PCR cDNA synthesis kit and PCR Select cDNA subtraction kit. The manufacturer's instructions were followed for both kits. All diagnostic steps were followed as recommended by the manufacturers. All PCR reactions were done using an Applied Biosystems 9700 with 96-well block, using Applied Biosystems plastics. Driver and tester populations used for subtraction were as below:

| subtracted cDNA | tester | driver |
|---|---|---|
| Subtracted macrophage | macrophage (hypoxia) | macrophage (normoxia) |
| Subtracted cardiomyoblast | cardiomyoblast (hypoxia) | cardiomyoblast (normoxia) |

The final subtracted cDNA samples were evaluated by performing RT-PCR using the following primers for human beta actin:

| | |
|---|---|
| sense: | TCACCCACACTGTGCCCATCTACGA |
| antisense: | CAGCGGAACCGCTCATTGCCAAATGG |

This showed that an additional 5 cycles of PCR were required to achieve similar levels of beta actin product from subtracted compared to unsubtracted cDNA, indicating a significant reduction in the representation of a non-regulated gene in the subtracted cDNA. Glyceraldehyde 3-Phosphate dehydrogenase PCR primers, as contained in the kit, were not used.

The three subtracted cDNA populations were ligated into a plasmid vector (pCRII, Invitrogen) to generate libraries, which were transformed into E.coli (INVαF′, Invitrogen) and plated out onto agar, supplemented with ampicillin and X-Gal, according to standard methods.

Colonies that are white indicate the presence of a recombinant plasmid, and these were picked into individual wells of 96-well plates containing 100 microliters LB-Ampicillin, and given 3-8 hr growth at 37 degrees. In this way, for each library, up to 15×96-well plates of clones were generated.

To screen clones for the presence of differentially expressed genes, replicate arrays of plasmid insert DNA were generated on nylon membranes: Firstly, PCR was performed using nested PCR primers 2R and 1, which flank the cDNA insert of each clone (sequence described in the PCR Select kit). The reaction mix also contains 200 uM d(A,T,C,G)TP, Advantage2 polymerase mix (Clontech Laboratories) and supplied 10× buffer. 40 ul reactions were set up in 96-well PCR reaction plates and inoculated with 0.5 ul bacteria from the library plates. 23 cycles of PCR were performed (95 degrees 10 sec; 68 degrees 2 min), and a selection of wells were checked on an agarose gel. In this manner a 96-well plate of insert DNA was generated for each 96-well plate of bacterial clones. Arrays of insert DNA were generated by transferring 4 ul of each well to 384-well plates (Genetix), and denaturing the DNA by adding 4 ul 0.4M NaOH and incubating at 37 degrees for 15 minutes. Bromophenol blue was added to the wells to allow visualisation of arraying. A 384-pin replicator (Genetix) was used to spot small volumes of denatured insert DNA onto dry nylon membranes (Hybond N+, AmershamPharmacia).

By repeating this operation from the same 384-well plate onto several membranes, matched pairs of membranes were produced, suitable for array screening. A fragment of the beta actin gene was spotted at specific positions of the arrays. Following spotting, the membranes were left at room temperature for 2 hr, re-denatured by placing on chromatography paper wetted with 0.3 M NaOH, neutralized by placing on chromatography paper wetted with 0.5 M Tris pH 7.5, dried at room temperature for 2 hr and crosslinked by exposing to 2000 joules WV radiation. Prior to hybridization, residual salts were removed from the arrays, by washing in hot 0.5% SDS.

Matched pairs of membranes were hybridised with subtracted cDNA samples; from hypoxic and normoxic cells, to determine the abundance of the genes corresponding to each spotted clone in the cDNA samples. Because the cDNA probes were subtracted, large differences in the hybridization signal for individual spots were apparent, which can be identified by eye. Prior to probe labelling, subtracted cDNA samples were digested with RsaI and run through Qiagen Qiaquick PCR purification columns to remove adapter sequences added during the PCR Select procedure. 25 ng cDNA was labelled with 33P using a commercial kit following the manufacturer's instructions (Promega, Prime-a-gene kit), and unincorporated label was removed using BioRad Biospin-6 columns following adding 2.5 ug yeast tRNA carrier.

Pre-hybridization, hybridization and washes were performed essentially according to the Research Genetics GeneFilters protocol, but supplementing the hybridization mixture with 10 ug of a cocktail of oligonucleotides complementary to the Clontech PCR Select nested PCR primers (equimolar mix of primers 1 and 2R and their reverse complements).

Hybridized arrays were exposed to X-ray film or were exposed to a phosphorimager (Molecular Dynamics, Storm) and clones showing gross differences in the hybridization signals with hypoxic compared to normoxic cDNA probes were identified. This procedure was used to process all clones originally picked from the primary libraries and PCR amplified. The selected clones were grouped together onto a single array (referred to here as a secondary array), and were re-screened with cDNA probes which had not been subtracted, to allow a more quantitative though less sensitive, evaluation of the relative abundance of the genes in hypoxia vs. normoxia.

In this case, probes were ds cDNA generated from the Clontech SMART cDNA synthesis kit (labelled using the Promega Prime-a-gene kit) or were total RNA (labelled according to the Research Genetics GeneFilters protocol), and hybridizations were done according to the Research Genetics GeneFilters protocol.

Hybridization signals were measured using a phosphorimager and were processed with ArrayVision (Imaging Research Inc) software using multiple beta-actin spots to normalize the quantitation and individual spot background correction. At this stage, the inserts of clones showing consistent up-regulation in hypoxia were sequenced using the 2R primer.

The identity of the genes were determined using BLAST at the NCBI (NLM, NIH) against the non-redundant data base collection. Where significant matches to human genes were not made, the human EST database was used. For both EST and non-EST hits, identifier numbers were also obtained from the UniGene database.

The above strategy was used independently for libraries derived from macrophages and from cardiomyoblasts. By screening a relatively large number of clones (several thousand per library), single genes were identified from multiple clones from any individual library. Multiple clones covered either the same or different regions of the genes.

In the above manner, certain hypoxia-inducible genes were identified from clones only derived from the cardiomyoblast library. These genes are listed in Table 1. Certain hypoxia-inducible genes were identified from clones only derived from the macrophage libraries. These genes are listed in Table 2. Certain hypoxia-inducible genes were identified from clones derived from both macrophage and myoblast libraries. These genes are listed in Table 3.

It can be seen that Table 3 contains many less genes than either Tables 1 and 2; demonstrating that these cell types have large differences in the genes induced by hypoxia. Importantly, the subtracted libraries for macrophage and cardiomyoblast were constructed in parallel. Therefore, major differences in the spectrum of genes isolated from these libraries are likely to be due to differences in the starting material, rather than due to technical differences in the production of the libraries. Importantly, the genes contained in these tables were confirmed to be hypoxia-regulated in the relevant cell type(s) by the described two-stage array hybridization screening process.

From Table 3 it is clear that although this subset of genes was found in subtracted libraries from both hypoxic macrophages and cardiomyoblasts, the fold-induction obtained between hypoxia and normoxia, for the different tissues differs widely. For the first 5 genes in this table, the hypoxia response is greater for macrophages, whereas for the last 2 genes it is greater for cardiomyoblasts.

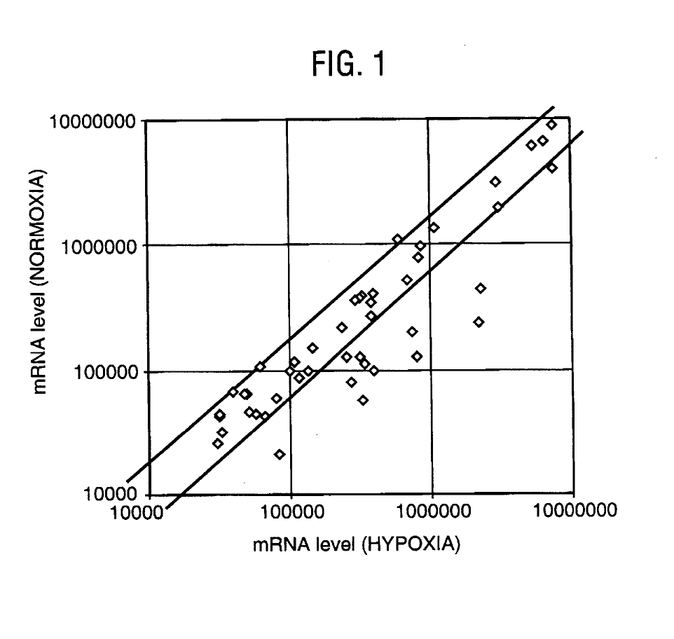

To test whether genes isolated only in the macrophage-derived subtracted libraries are not responsive to hypoxia in cardiomyoblast, cardiomyoblast cDNA isolated from normoxic and hypoxic cells was hybridised to an array of macrophage-derived clones. These data are presented as a scatter plot, showing normalized signal intensities in hypoxia versus normoxia, with each dot representing a single gene on the array. This plot is presented in FIG. 1. A gene that is not affected by hypoxia will localize around the y=x line, running diagonally through the centre of the graph. From the figure, it can be seen that most genes lie in this region, even though all the genes were responsive to hypoxia in the macrophage (Table 2). There is a subset of genes that lie beneath this region (x>y), representing induction of these genes by hypoxia in the cardiomyoblast.

Sequence data for the cDNA inserts of clones from our custom subtracted cDNA libraries is available. These are usually short fragments of 300-1000 bp. Some have been resequenced to obtain an accurate full insert sequence (see document “gene sequences/analysis”).

Several of the genes presented in Tables 1-3 encode hypothetical proteins of unknown function and others have no database matches with protein coding sequence. The work presented here provides some functional annotation for these genes, and potential applications for the treatment of disease. Certain genes, in particular the glycolytic enzymes and transporters, have been hypothesised previously as forming part of the generic hypoxia response. The data provided herein provide solid, validating data for these hypotheses.

It was surprising to note that cells from our cultures of human ventricle-derived cells, showing a cardiomyoblast-like phenotype, do not support significant induction of the following genes: Lactate dehydrogenase A,, Enolase 1, Phosphoglycerate kinase 1, Triosephosphate isomerase 1. These genes have been identified as being targets of the “ubiquitous” transcription factor HIF-1 alpha (“HIF-1: mediator of physiological and pathophsiological responses to hypoxia” J.Appl.Physiol 88: 1474-1480 (2000)).

### Example 1b

Preparation of Custom Array

To confirm the findings presented in Example 1a, and to obtain more accurate and additional data, both the subtracted cDNA library clones and the IMAGE clones identified from the Research Genetics Human GeneFilters have now been fabricated by the authors into an independently produced and verified gene array (referred to herein as the “custom gene array”), composed of PCR-amplified insert DNA. The methods used to produce this array are common in the art, but the key points are summarised below.

Clones from the subtracted cDNA library were PCR amplified as defined in Example 1a. In many cases, there were multiple cDNA clones corresponding to different regions of the same gene, and all these were represented on the custom gene array. IMAGE clones were obtained from the UK MRC HGMP Resource Centre (Hinxton, Cambridge CB101 SB, UK) and were re-isolated as individual colonies and sequenced to verify the correct identity of the clone. In the majority of cases, the same IMAGE clone identified from the Research Genetics Human GeneFilters was selected, but in some instances these clones were not available and alternatives were selected, corresponding to the same gene.

Additional genes, with well-defined roles in various disease processes relevant to hypoxia, were also represented on the array, as derived from IMAGE clones. It is well established in the literature that genes with similar functions are often co-regulated at the mRNA level, as determined by microarray data clustering methods (Iyer V R et al, Science. 1999 283(5398):83-7; Eisen M B et al Proc Natl Acad Sci USA. 1998 95(25):14863-8). This allows associations to be made between genes of unknown function (as present in the current specification) to genes of well defined function, in order to add significance to the former.

Normalisation is a key issue in array analysis. The custom gene array is a single colour type array, and contains a selection of additional IMAGE clones corresponding to genes which were empirically determined not to be affected by hypoxia and which are highly expressed in a wide range of human tissues and cell types. During data analysis, spot intensities were divided by the mean of all the reference genes shown below, each of which was present in quadruplicate on each array.

| Gene | IMAGE clone Acc. |
|---|---|
| FLJ11102 fis clone PLACE1005646 | AA464704 |
| matrix Gla protein | AA155913 |
| guanine nucleotide binding protein alpha stimulating 1 | R43581 |
| DKFZp434A1319 | W74725 |
| cDNA FLJ23280 fis clone HEP07194 | AA669443 |
| beta actin | (in house clone) |
| EF1a-like protein | AI817566 |
| ribosomal protein L37a | W91881 |

IMAGE clone plasmid miniprep DNA was prepared and PCR amplified with flanking vector primers of the sequences GTTTTCCCAGTCACGACGTTG and TGAGCGGATAACAATTTCACACAG. This was then purified and concentrated by ethanol precipitation, and the presence of a single band and DNA concentration were determined by agarose gel electrophoresis and by digital imaging methods.

Purified PCR product corresponding to all the clones (IMAGE and non-IMAGE) were normalized to 0.5 mg/ml by dilution. Arrays were fabricated onto Hybond N+ (Amersham) membranes using a BioRobotics TAS arrayer (Biorobotics, Cambridge CB37LW, UK) with a 500 micron pin tool. Using 384-well source plates and a 2×2 arraying format this array was relatively low density, thereby eliminating problems of spot-to-spot signal bleed. Also the large pin size and high source plate DNA concentration improves the sensitivity of detection. Post-arraying denaturation/neutralisation was essentially as described by Bertucci F et al., 1999 (Oncogene 18: 3905-3912).

Total RNA was extracted from cells using RNeasy (Qiagen) and 7 micrograms RNA was labelled with 100 microCi 33P dCTP using 2 micrograms poly dT (10-20 mer) as primer in a reverse transcription reaction. First strand RNA was then degraded under alkaline contritions, and this was then neutralized with Tris HCl pH 8.0, and the labelled cDNA was purified using BioRad BioSpin-6 chromatography columns. Pre-hybridization was performed in 4 ml Research Genetics MicroHyb solution supplemented with 10 micrograms poly dA (10-20 mer) and 10 micrograms Cot-1 DNA, at 45 degrees for 2-3 hours. The cDNA was then denatured by heating and added to the pre-hybridization, which was continued for 18-20 hr. Washing steps were done as follows: 2×SSC/1% SDS 2×20 min at 50 degrees and 0.5×SSC/1% SDS 10 min at 55 degrees. Arrays were exposed to Amersham Low Energy phosphor screens for 24 hr and scanned using a phosphorimager at 50 micron resolution. Image analysis was done using ArrayVision software (Imaging Research Inc). Tab delimited data files were exported and a full analysis performed using GeneSpring software (Silicon Genetics).

Using the described methodology a dynamic range of detection of 4 logs and a sensitivity of at least 1/50,000 is obtained, as determined by spike doping titration experiments. Having several technical differences compared to the Research Genetics Human GeneFilters as used in the initial filing, data from the custom gene array is expected to be quantitatively different.

### Example 1c

Hypoxia Regulation of Gene Expression in Macrophages by Exposing Cells to Hypoxia +/− Additional Signal Amplification.

The transcription factor HIF-1α, is ubiquitously present in cells and is responsible for the induction of a number of genes in response to hypoxia. This protein is considered a master regulator of oxygen homeostasis (see, for example, Semenza, (1998) Curr. Op. Genetics and Dev. 8:588-594). Although HIF-1a is well known to mediate responses to hypoxia, other transcription factors are also known or suspected to be involved. These include a protein called endothelial PAS domain protein 1 (EPAS1) or HIF-2a, which shares 48% sequence identity with HIF-1a (Tian H, et al. Genes Dev. 1997 11:72-82.). Evidence suggests that EPAS1 is especially important in mediating the hypoxia-response in certain cell types, and it is clearly detectable in human macrophages, suggesting a role in this cell type (Griffiths et al., 2000, Gene Ther., 7(3):255-62).

As supporting evidence for the hypoxic regulation of the genes contained within this specification, adenoviral vectors were used to overexpress HIF-1α and EPAS1 in primary human macrophages prior to exposure to hypoxia, in order to amplify the response. Because the role of these transcription factors as mediators of the hypoxia response is very well established, any further increases in the inducibility of specific genes resulting from this approach represents credible supporting evidence that those genes are responsive to hypoxia.

A commercially available system was used herein to produce adenoviral particles involving the adenoviral transfer vector AdApt, the adenoviral genome plasmid AdEasy and the packaging cell line Per-c6 (Crucell, Leiden, The Netherlands). The standard manufacturer's instructions were followed. Three derivatives of the AdApt transfer vector have been prepared, named AdApt ires-GFP, AdApt HIF-1a-ires-GFP and AdApt EPAS1-ires-GFP. In these vectors, for convenience, AdApt was modified such that inserted genes (i.e. HIF-1a or EPAS1) expressed from the powerful cytomegalovirus (CMV) promoter were linked to the green fluorescent protein (gfp) marker, by virtue of an internal ribosome entry site (ires). Therefore presence of green fluorescence provides a convenient indicator of viral expression of HIF-1a or EPAS1 in transduced mammalian cells. The control vector AdApt ires-GFP was used to allow discrimination between effects of the inserted genes (i.e. HIF-1a or EPAS1) to that of potential non-specific effects of adenoviral transduction or GFP expression. Standard subdloning methods were used to construct the adenoviral constructs as described in detail elsewhere (see co-pending, co-owned International patent application PCT/GB01/00758; Example 2).

The adenoviral transfer vectors AdApt HIF-1a-ires-GFP and AdApt EPAS1-ires-GFP, were verified prior to production of adenoviral particles, for their ability to drive expression of functionally active HIF-1a or EPAS1 protein from the CMV promoter in mammalian cells. This was achieved by transient transfection luciferase-reporter assays as described (Boast K et al Hum Gene Ther. Sep. 1, 1999;10:2197-208).

Using the aforementioned Introgene adenoviral system, caesium-banded, pure adenoviral particles were produced for each of the vectors AdApt ires-GFP, AdApt HIF-1a-ires-GFP and AdApt EPAS1-ires-GFP. Following the Introgene manual, adenoviral preparations were quantitated by spectrophotometry, yielding values of viral particles (VP) per milliliter.

Primary human macrophages isolated as described above, were washed and resuspended in DMEM (Gibco, Paisley, UK) supplemented with 4% fetal bovine serum (Sigma). 5×10⁶ cells were plated into nine individual 10 cm Primeria (Falcon) tissue culture dishes containing medium plus adenovirus as shown below (experimental design), to give a total volume of 10 ml per plate. Two doses of adenovirus were used; 5.3×10⁸ viral particles/ml (low) and 1.6×10⁹ viral particles/ml (high). These amounts were chosen following a series of titration experiments. Following culture for 16 hr, during which the macrophages adhere to the plate and are infected by the adenoviral particles, the medium was removed and replaced by IMDM medium (Gibco, Paisley, UK) supplemented with 2% human AB serum. A further 24 hr period of culture was allowed prior to experimentation, to allow gene expression from the transduced adenovirus. Gene transduction was verified by visually assessing gfp expression and expression of the viral HIF-1a and EPAS1 genes was determined by real time quantitative RT-PCR using an ABI Prism 7700 TaqMan and CyberGreen protocol. For the high doses of virus, the total levels of HIF-1a or EPAS1 mRNA present in the transduced cells were increased by 10-30 fold.

For experimentation with conditions of hypoxia, identical culture dishes were divided into two separate incubators: One at 37 degrees, 5% CO2, 95% air (=Normnoxia; equivalent to 20% Oxygen) and the other at 37 degrees, 5% CO2, 94.9% Nitrogen, 0.1% Oxygen (=Hypoxia). After 6 hours culture under these conditions, the dishes were removed from the incubator, placed on a chilled platform, washed in cold PBS and total RNA was extracted using RNeasy (Qiagen) following the manufacturer's instructions.

Experimental Design

| | | Adenovirus | |
|---|---|---|---|
| | | amount | |
| | Adenovirus | (low = 5.3 × 10⁸ vp/ml | Oxygen |
| Condition | (type) | high = 1.6 × 10⁹ vp/ml) | (%) |
| 1 | none | none | 20 |
| 2 | AdApt ires-GFP | low | 20 |
| 3 | AdApt ires-GFP | high | 20 |
| 4 | AdApt ires-GFP | low | 0.1 |
| 5 | AdApt ires-GFP | high | 0.1 |
| 6 | AdApt HIF-1a-ires-GFP | low | 0.1 |
| 7 | AdApt HIF-1a-ires-GFP | high | 0.1 |
| 8 | AdApt EPAS1-ires-GFP | low | 0.1 |
| 9 | AdApt EPAS1-ires-GFP | high | 0.1 |

RNA samples from the experimental conditions shown above were each hybridised to individual copies of the Custom gene array and processed as described earlier. To ensure reproducible data, this was repeated so each RNA sample was hybridised to 4 separate arrays. Therefore a total of 36 arrays were used for this experiment. Data analysis was done taking the mean signal of each spot from the four array replicates of each RNA sample. When displayed graphically, standard error of the mean is displayed as the error bar. Expression values were calculated so that they represent the fold-change ratio as compared to conditional, i.e. untreated cells.

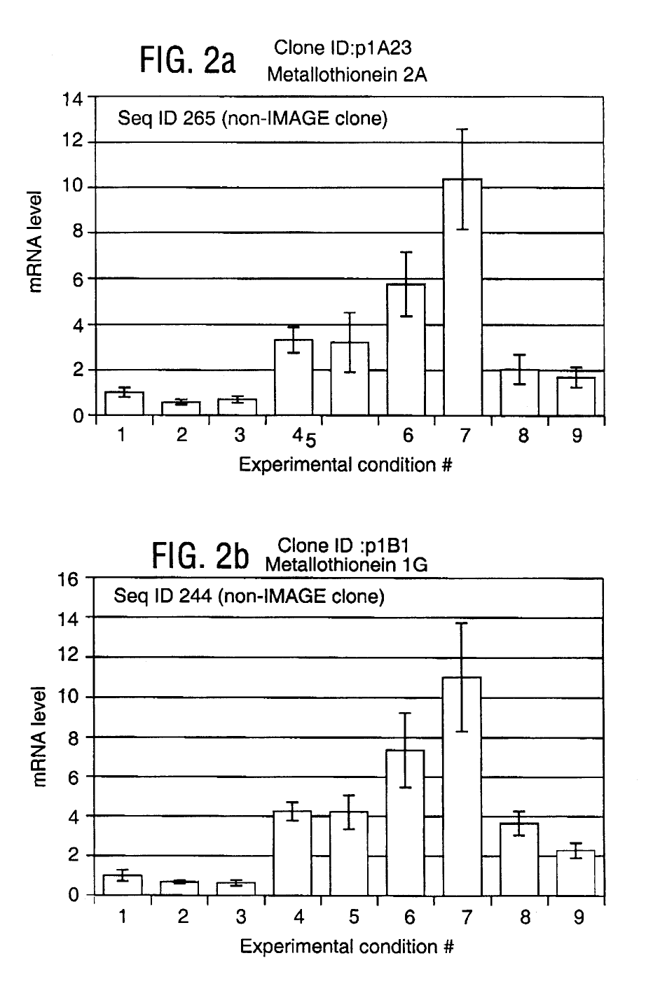

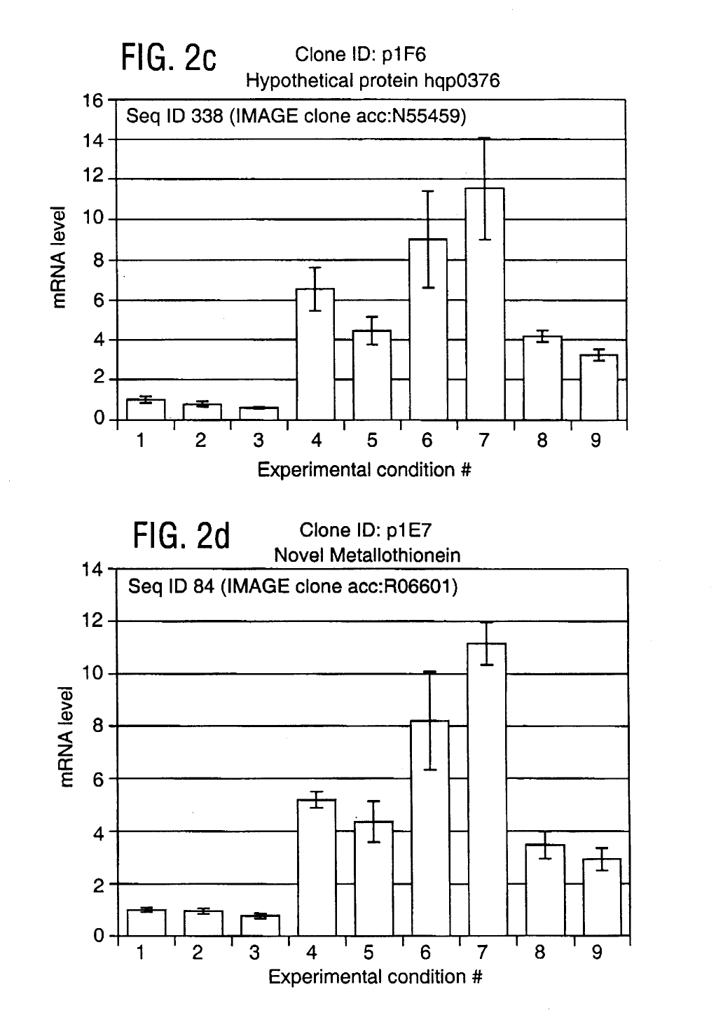

For genes shown in Table 4 it can be seen that in cells transduced by the control adenovirus AdApt ires-GFP there is a response to hypoxia (conditions 4,5) as compared to in normoxia (conditions 2,3). However this response is significantly greater when the natural hypoxia response is amplified by overexpression of HIF-1alpha from the adenovirus AdApt HIF-1a-ires-GFP (conditions 6,7). Furthermore, this effect is usually dependent on the amount of HIF1alpha overexpression (i.e. greater in condition 7 compared to 6). This same data is displayed graphically in FIG. 2. It can be seen that these genes encode metallothionein proteins. One of these (Nucleotide SEQ ID NO: No. 84; Protein SEQ ID NO: No. 83) is a novel member of the matallothionein family. Several metallothionein genes are known in the art to be activated by hypoxia, supporting the usefulness of this data.

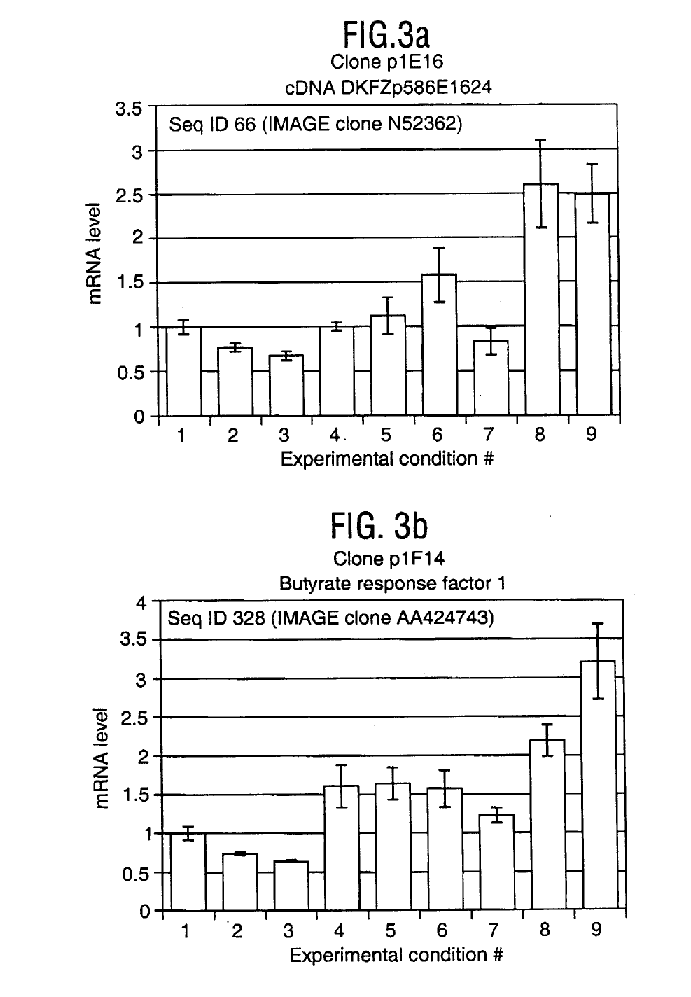

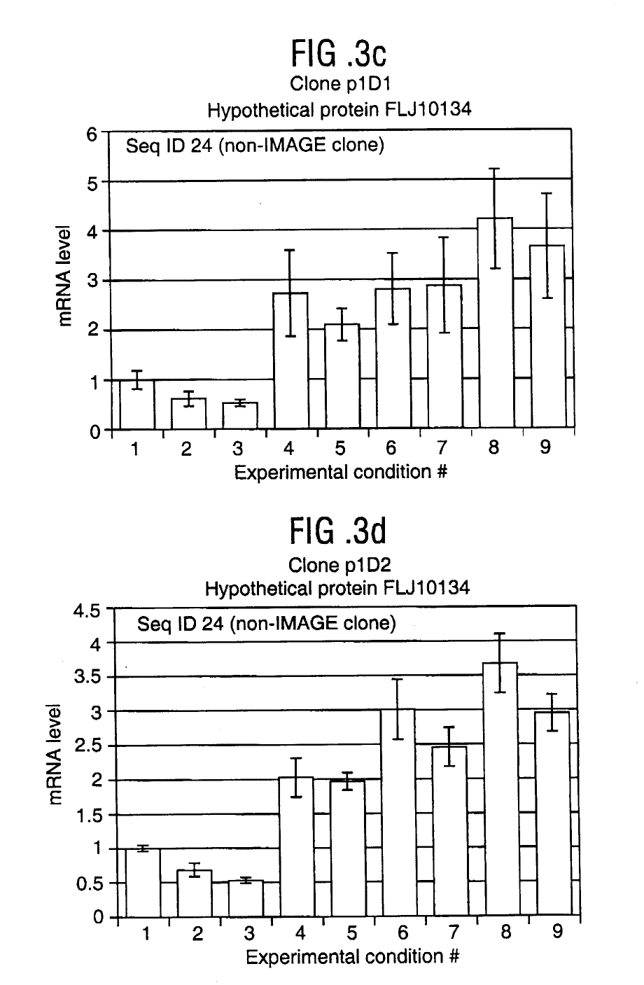

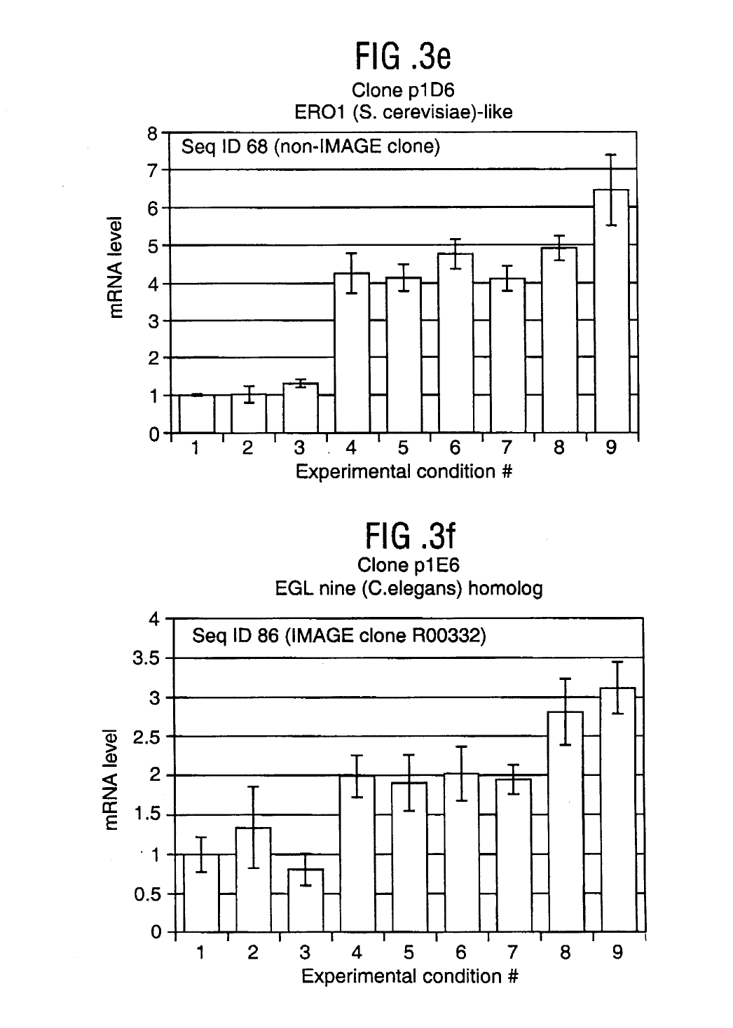

For genes shown in Table 5 and FIG. 3 it can be seen that in cells transduced by the control adenovirus AdApt ires-GFP there is a response to hypoxia (conditions 4,5) as compared to in normoxia (conditions 2,3). However this response is significantly greater when the natural hypoxia response is amplified by overexpression of EPAS1, from the adenovirus AdApt EPAS1-ires-GFP (conditions 8,9).

In the case of the protein encoded by SEQ ID NO: No. 24, results are available independently for two separate cDNA clones representing non-overlapping regions of the same full length gene.

In the case of the protein encoded by SEQ ID NO: No. 86 (EGL nine (C.elegans) homolog 3), additional evidence is described above in support of the function of this protein. Furthermore, real time quantitative RT-PCR analysis of this gene using an ABI Prism 7700 TaqMan and CyberGreen protocol, has been performed, to verify and more accurately quantitate the upregulation of EGL nine (C.elegans) homolog 3 in response to hypoxia and EPAS1, adenoviral overexpression. The main difference between the array-based and real time quantitative RT-PCR methodologies is that the latter is far more sensitive and therefore can detect expression in the off-state (here normoxia) for weakly expressed genes. This data has shown an induction ratio of 819-fold for EGL nine (C.elegans) homolog 3 in response to hypoxia with additional EPAS1 expression, from RNA generated from an independent experiment. This data was normalized to beta actin.

Similarly another weakly-expressed EPAS1-induced gene, Semaphorin 4b (SEQ ID NO: No. 91/92; see additional discussion above) has been determined using real time quantitative RT-PCR methodology, showing an actin-normalized induction ratio of 30.1 is found (data not shown).

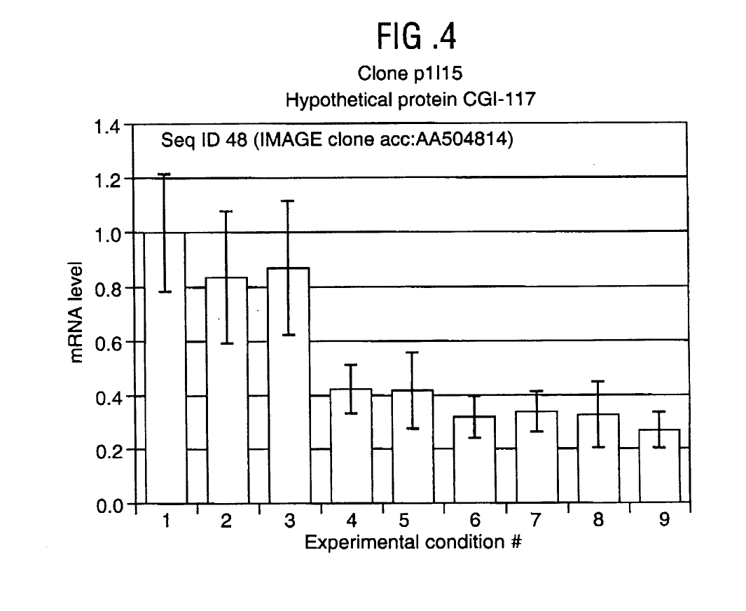

For the gene shown in Table 6 and FIG. 4 it can be seen that in cells transduced by the control adenovirus AdApt ires-GFP, there is a negative response to hypoxia (conditions 4,5) as compared to in normoxia (conditions 2,3). However, this response is significantly greater when the natural hypoxia response is amplified by overexpression of HIF1alpha or EPAS1 (conditions 6,7,8,9).

### Example 2

Differences in the Hypoxia Responses of Resting and Activated Macrophages.

Macrophages accumulate at hypoxic areas in various disease states, including cancer, rheumatoid arthritis, atherosclerosis and wound healing. At these sites macrophages activation is liable to occur, such as in response to T-cell derived gamma interferon. For instance, in atherosclerotic plaques there is an accumulation of both T-cells and macrophages, and these are known to interact with one another (reviewed in Lusis A J, Atherosclerosis. Nature. Sep. 14, 2000;407(6801):233-41).

It is well established that the macrophage has a significant role in the pathology of the above diseases involving hypoxia, and that most functions of the macrophage (including inflammatory functions) are greatly increased following activation. Therefore any therapeutic strategy aimed at the hypoxic macrophage, needs to also consider the effects of macrophage activation and possible cross talk between the responses to macrophage activation and hypoxia.

2.1: Research Genetics Human GeneFilters

This work was carried out using Research Genetics Human GeneFilters, which contain DNA derived from clones of the IMAGE cDNA collection, representing genes of varying degrees of characterization. A series of 6 arrays of human genes were used (GeneFilters GF200-205), potentially covering a total of 31,104 genes. Generally, single genes are represented only once in these arrays. However, sometimes IMAGE clones initially thought to represent separate genes, upon re-analysis were found to be different regions of the same gene. Here we have presented data for all clones individually, though they possess the same UniGene ID and gene name. An example is hypothetical protein FLJ20037.

The methodology for Research Genetics arrays is similar in principle to that described for the array screening of clones from subtracted libraries. There are several attributes to this method: Relatively small amounts of RNA can be labelled to make cDNA probes, in a single step reaction, and probes are labelled with the same chemical group (33P), so there are no errors introduced as a result of using different dyes, which may differ in stability etc. Using a Phosphorimager allows detection over a wide range of intensities (over 4 logs). Overall it is interesting to note a recent study, which has favourably re-evaluated the performance of the nylon based array, as compared with the glass-based microarray method (Bertucci F et al, Hum Mol Genet 8:1715-1722 (1999)).

Experiments were done essentially as described in the Research Genetics GeneFilters protocol. Duplicate copies of each array from the same production batch, were used and hybridised in parallel with labelled RNA isolated from normoxic and hypoxic primary human macrophages. Hybridised arrays were scanned twice using a Molecular Dynamics Storm phosphorimager, and both images were analysed to ensure reproducibility. Furthermore, the experiments were repeated using the same RNA samples, but with different array lot numbers, again to ensure reproducibility.

Analysis was performed using Research Genetics Pathways software, with normalisation using the ‘all data points’ option. Analyses were output as spreadsheets and filtered to remove data points where the signal intensity was less than 4-fold above the general background for the experimental condition with the higher signal (hypoxia or normoxia depending on whether hypoxia causes induction or repression). Sometimes expression in the lower state was not significantly above background, and the ratio will therefore be underestimated. Ratios were calculated by normalized signal intensity in hypoxia divided by normoxia. Changes were verified visually from the original array images.

In this manner, comparisons were made between normoxia and hypoxia in resting macrophages. The whole procedure was then repeated for activated macrophages, to investigate possible differences in the response to hypoxia. It is possible that potential differences for certain genes could be correlated with changes in expression resulting from activation, prior to challenge with hypoxia. To explore this possibility, comparisons were made between resting and activated macrophages, both in normoxia. Since some of the genes we have identified as being activated by hypoxia have very low hybridization signals in normoxia (for both resting and activated macrophages), this comparison was not possible.

We have found various patterns of gene expression changes occurring in response to hypoxia, related to the activation state of macrophages, which are presented below. Such a range of responses, specific to various subsets of genes, was not expected, and contradicts a view that the hypoxia response is a largely a generic mechanism.

Table 7 shows genes that are induced by hypoxia to a similar degree in resting and activated macrophages.

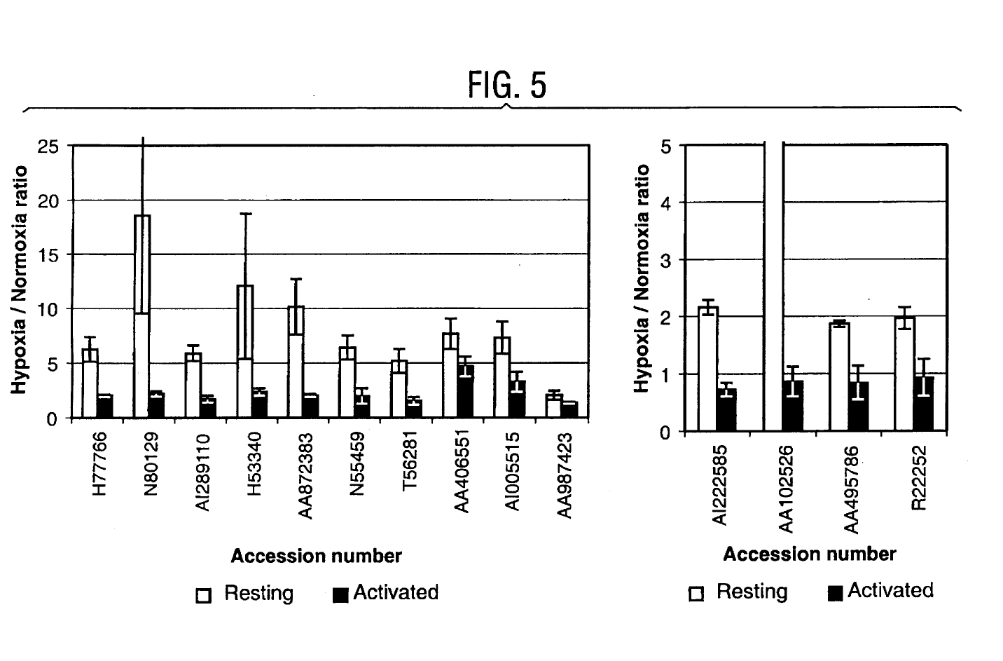

Table 8 shows genes that are induced by hypoxia to a greater degree in resting macrophages, as compared to activated macrophages. These data are presented illustratively in FIG. 5.

Data from Table 8/FIG. 5 reveals several unexpected observations.

A) From the final column it can be seen that macrophage activation in the absence of hypoxia, causes induction of many of these genes. This suggests that the signalling pathways resulting from activation and hypoxia might converge to a single transcriptional regulator, causing macrophage activation to pre-empt the response to subsequent hypoxia. This is exemplified most strikingly for Interleukin 8, which is dramatically induced in response to macrophage activation, but shows no additional response to hypoxia.

B) Genes in rows 11, 13 and 14 have no response to hypoxia following macrophage activation, though there is not a preceding large increase in expression in response to macrophage activation alone. This suggests that in the activated macrophage, the necessary signalling pathway or transcriptional regulator is not functional.

C) Although Table 8 was produced electronically, without selecting genes based on their names, it can be seen that genes encoding proteins of the metallothionein family feature strongly.

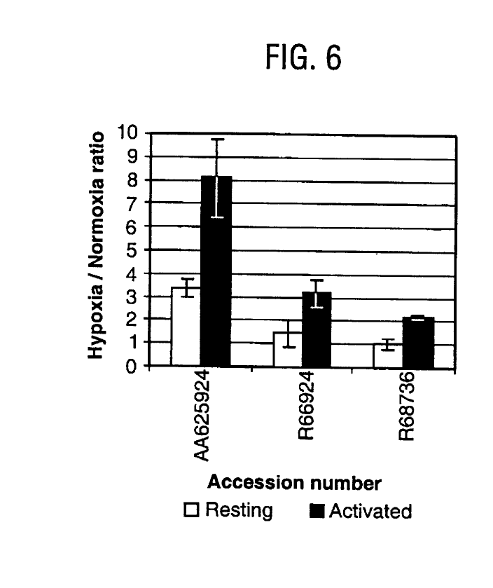

Table 9 shows genes which are induced by hypoxia to a greater degree in activated macrophages, compared to resting macrophages. These data are presented illustratively in FIG. 6.

In Table 7, there are several genes for which hypoxia/normoxia ratios were only obtained for activated macrophages, such as Cox-2 (see row 47). For these genes, macrophage activation usually increases expression of the gene to detectable levels, thus allowing the study of subsequent changes in response to hypoxia. It is likely that these genes are not significantly expressed in resting macrophages irrespective of hypoxia, and therefore the hypoxia response is probably specific to activated macrophages.

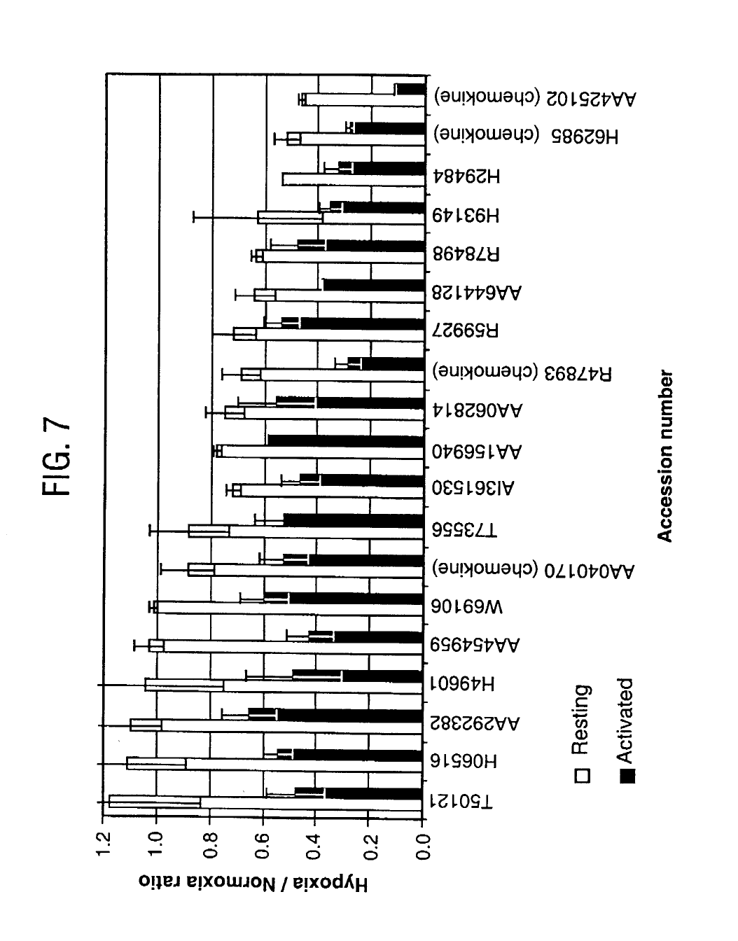

Certain genes respond to hypoxia by decreasing mRNA expression (repression), and these genes therefore have hypoxia/normoxia ratios of <1.0. This phenomenon is known in the field of hypoxia, although the mechanism is obscure. Data is presented in tables 7-9, which unexpectedly shows that this hypoxia-induced repression for specific genes is not a generic process, but is dependent on the cellular context. In Table 10/FIG. 7, genes are presented that are hypoxia-repressed to a greater degree in activated (column 7) compared with resting (column 8) macrophages. Prior to any hypoxic challenge, these gene are induced to varying degrees, in response to macrophage activation (column 9), suggesting a shared mechanism for these separate responses. From Table 10, genes in rows 1-6 show that macrophage activation is necessary to obtain any response to hypoxia. In resting macrophages, these genes are not responsive to hypoxia at all.

Strikingly, Table 10/FIG. 7 shows that seven separate genes encoding chemokine proteins (Monocyte chemotactic protein 1, Macrophage inflammatory protein 1b, Monocyte chemotactic protein 3 and Small inducible cytokine A3, Monocyte chemotactic protein 2, Macrophage inflammatory protein 2a and Macrophage inflammatory protein 2 precursor) are more strongly repressed in activated macrophages as compared to resting macrophages. These genes are also among the most inducible in response to activation alone, in normoxia (column 9). These findings are of potential utility in view of the great significance of chemokines to inflammatory disease. For example, macrophage chemotactic factor 1 (Table 10, row 19) is key to the pathological role of the macrophage in atherosclerosis (“Chemokines and atherosclerosis” Reape T J and Groot P H E, Atherosclerosis 147: 213-225, 1999).

Genes in rows 20-30 of Table 10, were not detectably expressed in resting macrophages, irrespective of hypoxia. Table 11 shows other genes that were down-regulated in response to hypoxia in macrophages.

### Example 3

Tissue-specific Hypoxia Regulation of Gene Expression by an Analysis of a Series of Primary Human Cell Cultures.

Equivalent cultures of non-immortalized, non-transformed primary human cells of 10 distinct types, were cultured in either normoxia or were exposed to hypoxia for 6 hr and 18 hr, and gene expression changes were determined. To the inventors' knowledge, this is the first time that such a study has been reported. Moreover, unlike the vast majority of information in the public domain relating to genes responsive to hypoxia, all of these cells were human and were cultured without any modifications following isolation from the human donors. By using primary cells rather than cell lines or immortalized cultures, the findings of this work more accurately represents the situation in the human body.

Most cell types were obtained from Clonetics (distributed by BioWhittaker, Walkersville, Md.) and cultured according to the manufacturer's recommendations, unless where otherwise shown. #1:adipocyte (Clonetics CC-2568; derived from subcutaneous adult adipose tissue), #2:cardiomyocyte (Clonetics CC-2582; derived from fetal tissue; prior to experimentation cultured in minimal medium: DMEM, 4% Horse serum), #3:endothelial (TCS CellWorks ZHC-2101 human umbilical vein endothelial cells), #4:fibroblast (Clonetics CC-2511 dermal fibroblasts derived from adult tissue), #5:hepatocyte (Clonetics CC-2591, derived from adult tissue), #6:macrophage (derived from human blood as described elsewhere in the specification), #7:mammary epithelial (Clonetics CC-2551; derived from adult tissue), #8:monocyte (derived from human blood as described elsewhere in the specification but without the 7 day differentiation culture period), #9:neuroblastoma (neuroblastoma-derived cell line SH-SY5Y), #10:renal epithelial (Clonetics CC-2556; derived from fetal tissue), #11:skeletal muscle myocyte (Clonetics CC-2561; derived from adult tissue). A non-primary cell type (#9) was used to represent neurons, since primary human neurons are difficult to source. Therefore a total of 11 cell types are compared. It should be noted that RNA from hepatocytes at the 16 hr timepoint of hypoxia was not available for this work.

Genes which were induced or repressed preferentially in particular cell type(s) were identified by hybridization of the RNA samples to the custom gene array, as described in Examples 1b and 1c. Each RNA sample was hybridised to duplicate or triplicate arrays, to ensure reproducible data, and was analysed using GeneSpring software. Data from replicate arrays were merged during analysis to generate mean values. Data normalisation was achieved per-array using the aforementioned list of control genes, such that differences in RNA labelling or hybridization due to experimental variation were corrected by referencing each gene to the mean value of the reference genes on the same array. Also, for each gene, expression values were obtained which represent the value in each experimental condition (e.g. macrophages 6 hr hypoxia) as compared to the median of value of that gene throughout the full range of experimental conditions (i.e. from all cell types). This transformation does not alter the relative values of any gene between the different experimental conditions, and is done since these is no obvious single reference experimental condition to create ratio values. This is common in microarray data analysis.

Table 12 shows the full dataset of this analysis. From this it can be seen that certain genes respond to hypoxia differently, depending on the particular cell type. This information is valuable in identifying biological targets for the development of therapeutic and diagnostic products. Not only does it indicate a particularly significant role for these genes in the specific cell type implicated in a disease, but it also identifies that any therapeutic product is less likely to produce problematic toxicological effects. Data shown in Table 12 and the derived figures, are reproducible, and are an accurate determination of mRNA expression levels. This may be confirmed by independent means, such as quantitative real time RT-PCR.

Certain genes from Table 12 will be presented for illustration.

Genes with a Greater Response in Monocytes or Macrophages

Since monocytes and macrophages are similar cell types, the latter derived from the former, they will be analysed together.

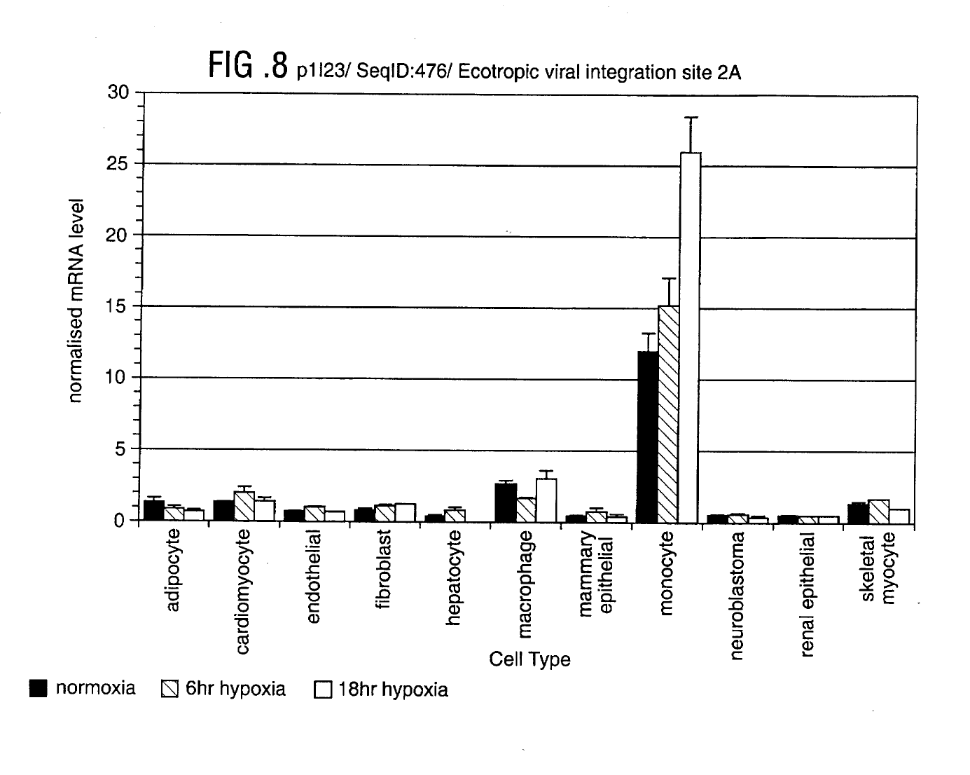

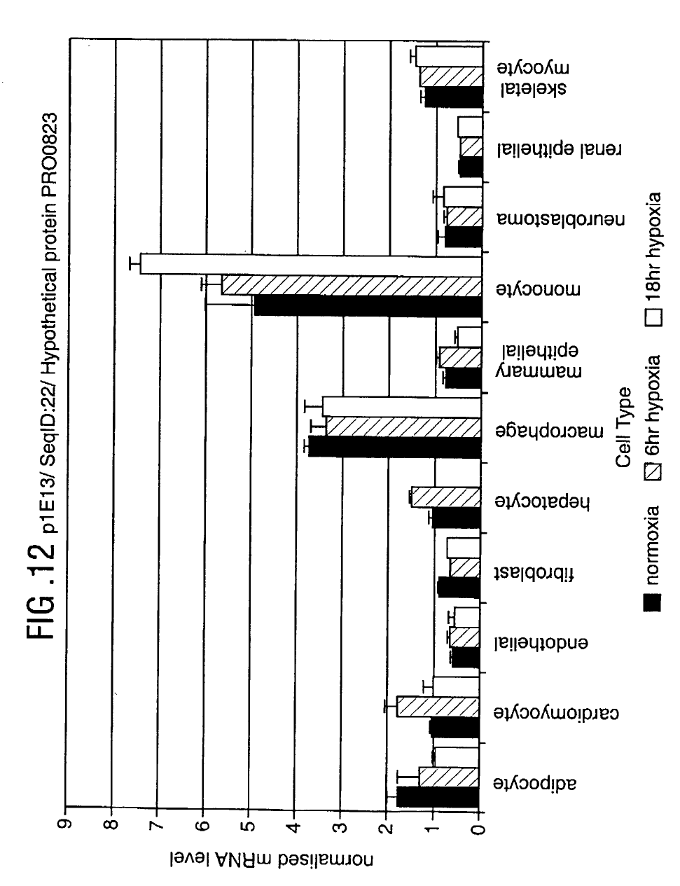

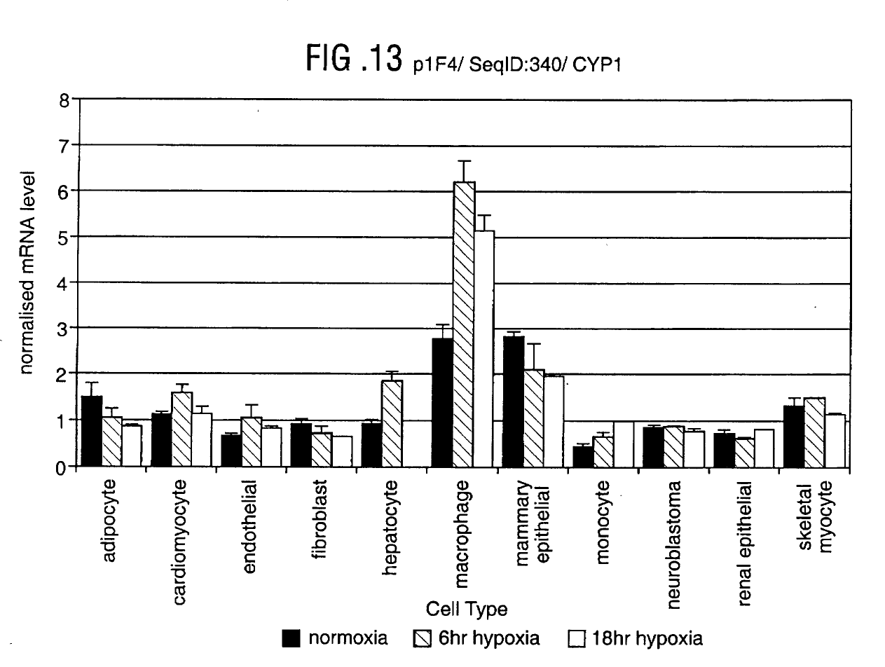

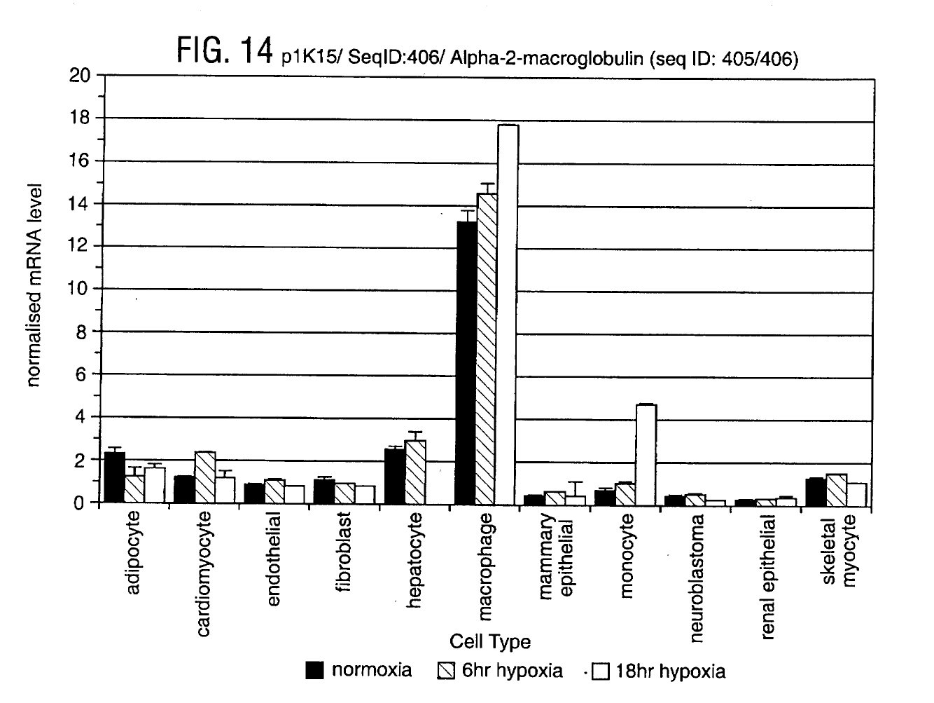

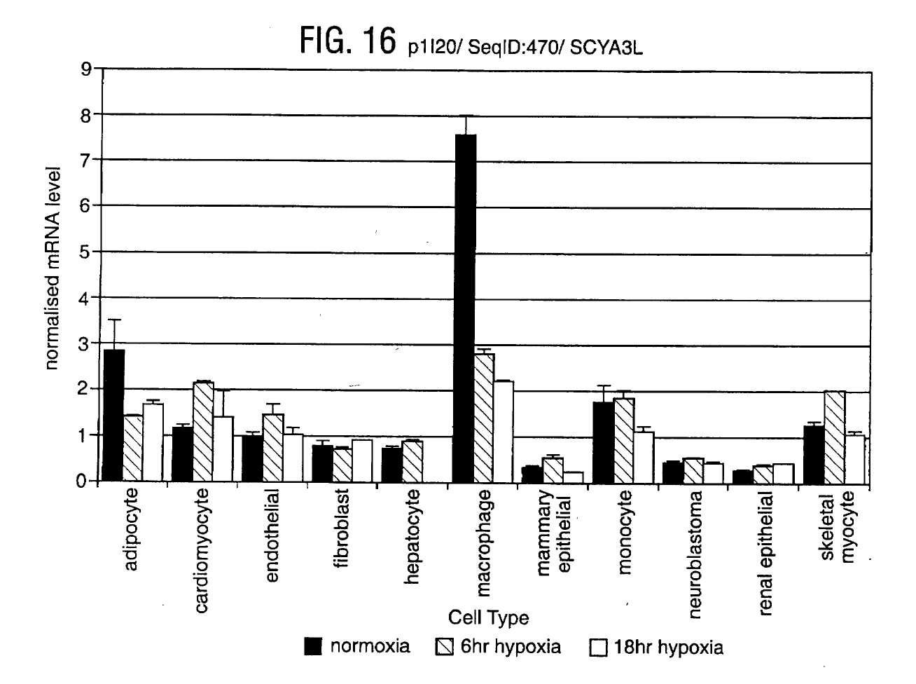

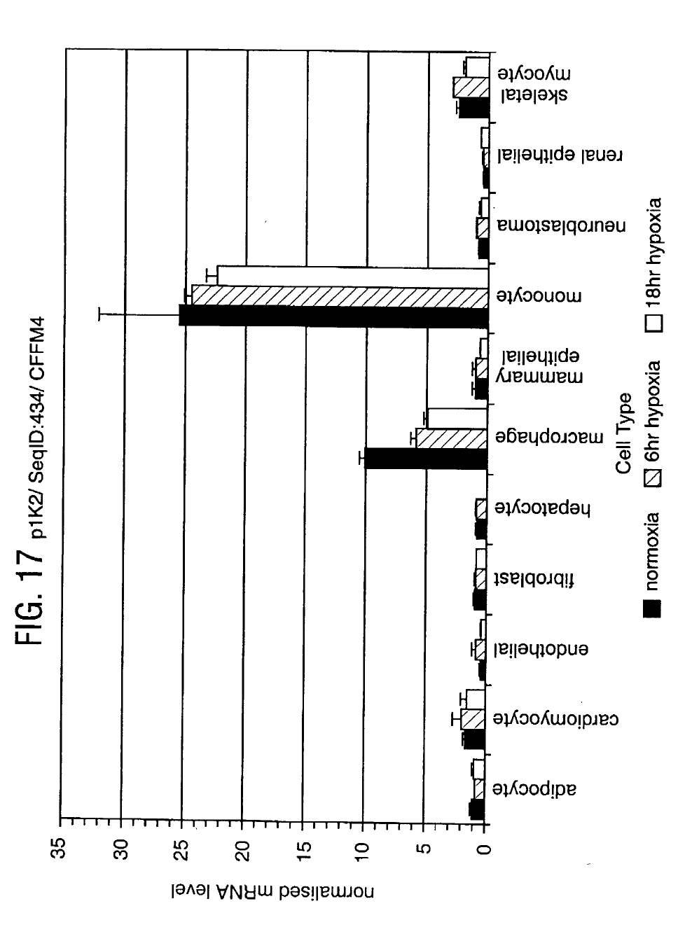

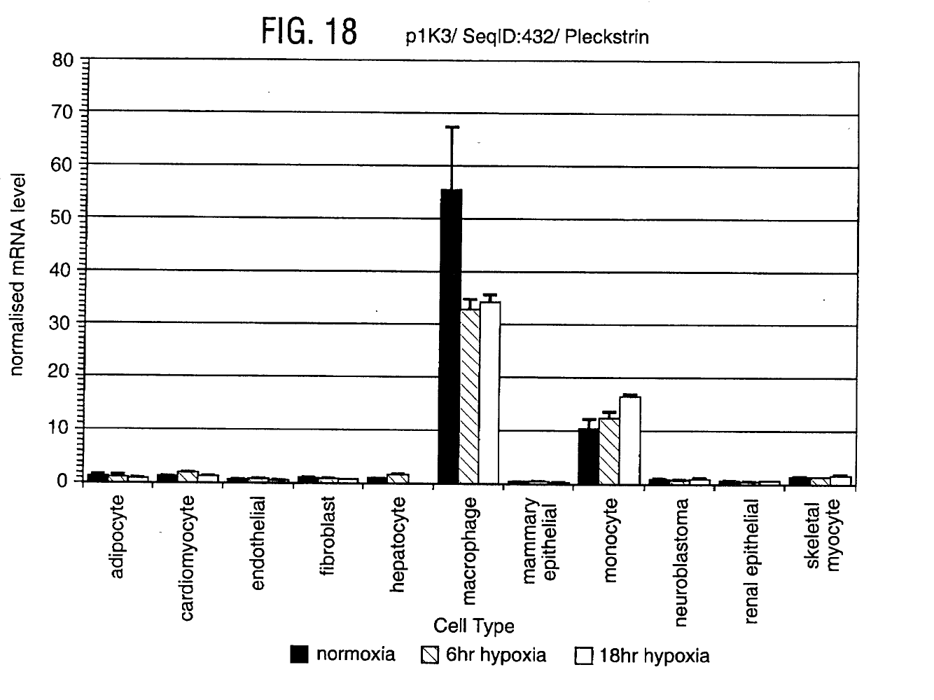

Expression profiles of 11 genes showing hypoxia-induced changes in gene expression which are most pronounced in monocytes or macrophages are shown in FIGS. 8-18. These genes correspond to:

| | |
|---|---|
| SEQ ID | CYP1 (cytochrome P450, subfamily XXVIIB) |
| NO::339/340 | |
| SEQ ID | interleukin 1 receptor antagonist |
| NO::357/358 | |
| SEQ ID | Regulator of G-protein signalling 1 |
| NO::375/376 | |
| SEQ ID | GM2 ganglioside activator protein |
| NO::389/390 | |
| SEQ ID | Alpha-2-macroglobulin |
| NO::405/406 | |
| SEQ ID | Ecotropic viral integration site 2A= |
| NO::475/476 | |
| SEQ ID | high affinity immunoglobulin epsilon receptor beta |
| NO::433/434 | (CFFM4) |
| SEQ ID | Pleckstrin |
| NO::431/432 | |
| SEQ ID | cytokine effector of inflammatory response SCYA3L |
| NO::469/470 | |
| SEQ ID | Novel PI-3-kinase adapter |
| NO::79/80 | |
| SEQ ID | hypothetical protein PRO0823 |
| NO::21/22 | |

It will be appreciated that the majority of these genes have a known biological function in immunity/inflammation, consistent with the known function of the monocyte/macrophage. Further to this knowledge, this data identifies that in hypoxic disease sites where monocyte/macrophages make up a significant proportion of the cell types, such as in rheumatoid arthritis synovial membranes, that these genes are possible therapeutic targets.

Ecotropic Viral Integration Site 2A (SEQ ID NO::475/476)

For example, the gene illustrated in FIG. 8, Ecotropic viral integration site 2A (SEQ ID NO::475/476) is induced in hypoxic monocytes to a level over 25 times higher than the median expression level of this gene throughout the other cell types. This gene, of unknown function, is located on Chromosome 17q11.2 close to genes with immune functions. Presented elsewhere in this specification is data showing that expression of Ecotropic viral integration site 2A is downregulated in response to the inflammatory cytokine interferon gamma. These novel data provide evidence that Ecotropic viral integration site 2A is a novel target for inflammatory conditions involving hypoxia and monocytes.

Novel PI-3-kinase Adapter SEQ ID NO::79/80 Clone p1E9 (EST Accession R62339).

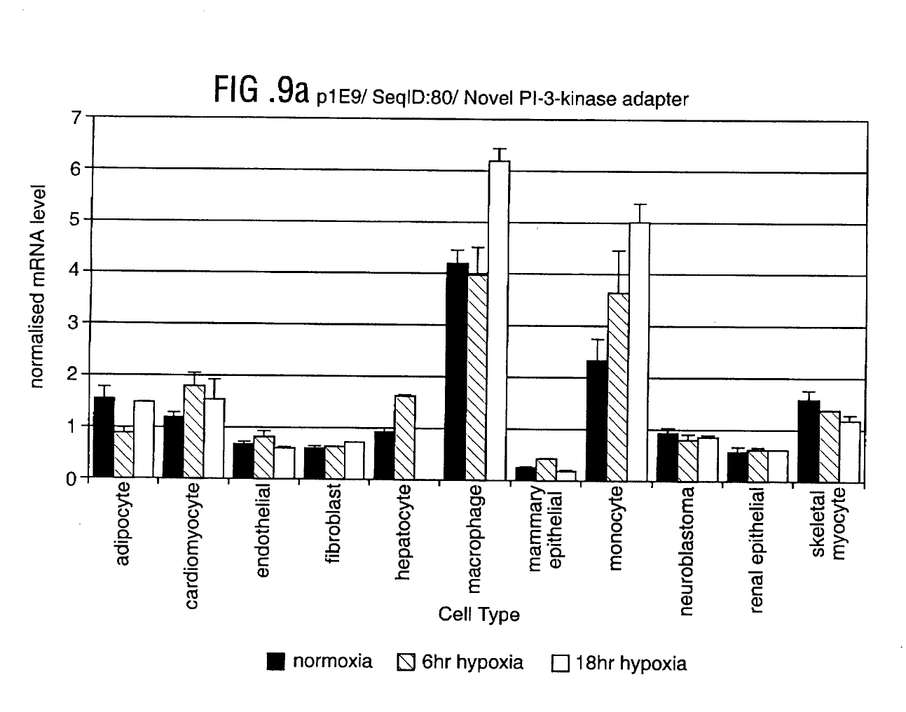

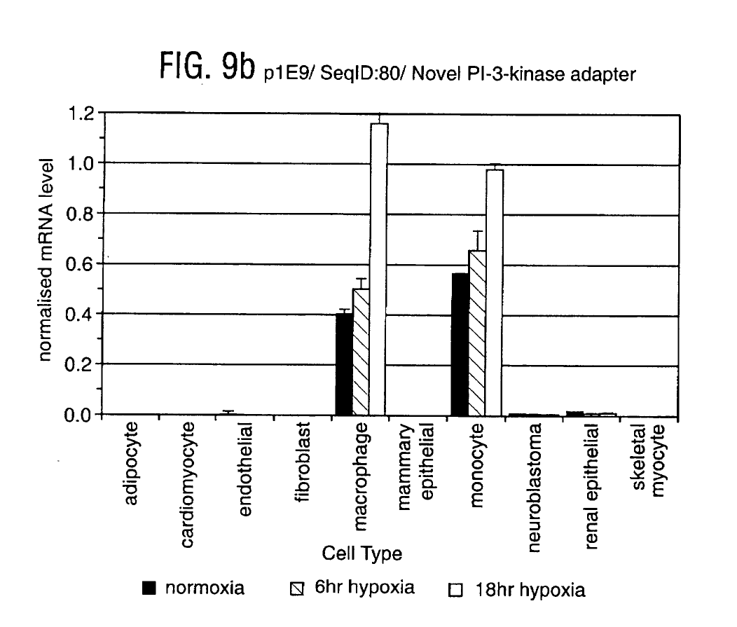

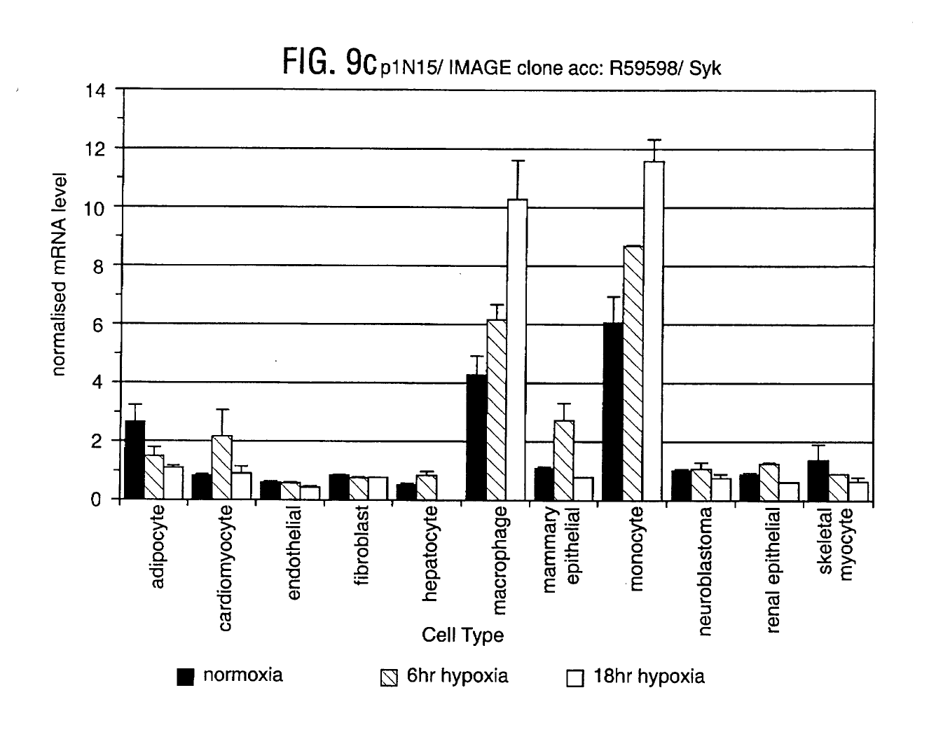

Another example, in FIG. 9a, is SEQ ID NO::79/80 (EST accession R62339). It is seen that in hypoxic macrophages, this gene is expressed at 6-fold higher levels than the median expression level of this gene throughout the other cell types. Therefore, the levels of the encoded protein in hypoxic monocytes/macrophages, as found at various disease sites, are likely to be higher than in other cell types not involved in the disease process or present at the site of disease. This illuminates a novel utility of this gene as a target for the development of therapeutic products for diseases involving monocytes/macrophages and hypoxia.

The data that led to the generation of this Figure are as follows:

| | | Normalized expression |
|---|---|---|
| Cell type | Oxygen | (clone p1E9/SeqID: 79/80) |
| adipocyte | normoxia | 1.54 |
| adipocyte | hypoxia 6 hr | 0.89 |
| adipocyte | hypoxia 18 hr | 1.48 |
| cardiomyocyte | normoxia | 1.18 |
| cardiomyocyte | hypoxia 6 hr | 1.80 |
| cardiomyocyte | hypoxia 18 hr | 1.53 |
| endothelial | normoxia | 0.68 |
| endothelial | hypoxia 6 hr | 0.82 |
| endothelial | hypoxia 18 hr | 0.60 |
| fibroblast | normoxia | 0.60 |
| fibroblast | hypoxia 6 hr | 0.64 |
| fibroblast | hypoxia 18 hr | 0.73 |
| hepatocyte | normoxia | 0.92 |
| hepatocyte | hypoxia 6 hr | 1.62 |
| macrophage | normoxia | 4.20 |
| macrophage | hypoxia 6 hr | 3.97 |
| macrophage | hypoxia 18 hr | 6.19 |
| mammary epithelial | normoxia | 0.25 |
| mammary epithelial | hypoxia 6 hr | 0.42 |
| mammary epithelial | hypoxia 18 hr | 0.18 |
| monocyte | normoxia | 2.33 |
| monocyte | hypoxia 6 hr | 3.63 |
| monocyte | hypoxia 18 hr | 5.01 |
| neuroblastoma | normoxia | 0.93 |
| neuroblastoma | hypoxia 6 hr | 0.80 |
| neuroblastoma | hypoxia 18 hr | 0.85 |
| renal epithelial | normoxia | 0.57 |
| renal epithelial | hypoxia 6 hr | 0.61 |
| renal epithelial | hypoxia 18 hr | 0.61 |
| skeletal myocyte | normoxia | 1.58 |
| skeletal myocyte | hypoxia 6 hr | 1.37 |
| skeletal myocyte | hypoxia 18 hr | 1.17 |

To substantiate the array-based data, the same RNA samples were examined by real time quantitative RT-PCR. The advantages of this method are that it is more sensitive and because two gene-specific primers are used, the data will be more specific to the gene in question.

RNA from the above samples (except for the hepatocyte RNA which was unavailable) was Dnase I-treated prior to reverse transcription to remove possible contaminating genomic DNA and was reverse transcribed using an oligo dT ₍₁₅₎ primer and Superscript II reverse transcriptase. These samples were used as template for PCR reactions using primers specific to EST accession R62339 or to beta-actin. Primer sequences were as follows:

Novel PI-3-kinase adapter SEQ ID NO::79/80 Clone p1E9 (EST accession R62339).

Forward Primer
5′ GCC CTT AGT TTT TCA CTT CTT CGT 3′
Reverse Primer
5′ CGT TAA GAT CCA TTC TCA TTG CTG AT 3′
Beta Actin
Forward Primer
5′ GCC CTG AGG CAC TCT TCC A 3
Reverse Primer
5′ GCG GAT GTC CAC GTC ACA 3′

All RT-PCR reactions were performed using an ABI Prism 7700 Sequence Detector system. For each Q-PCR run, a master mix was prepared with 2×SYBR Green I master mix (Applied Biosystems) and primers at 5 μM. Two microlitres of respective diluted cDNA were added to PCR master mixture, amounting to 25 μL. The thermal cycling conditions comprised 50° C. for 2 minutes, 95° C. for 10 minutes, 40 cycles at 95° C. for 15 seconds, and 60° C. for 1 minute. PCR reactions were set up in 96 well format with duplicate amplifications for each data point including 8 serial cDNA dilutions (0.2, 0.1, 0.05, 0.025, 0.01, 0.005, 0.001 and 0.0001) of macrophage treated with 18 hours hypoxia to compose a standard curve, a no template control, no amplification control lacking reverse transcriptase, and each cDNA sample at a dilution value of 0.1. The experiment for the novel PI3K adapter was carried out in triplicate for reproducibility which were later determined by linear regression analysis. Data was analysed with necessary adjustment of the default baseline and threshold line using ABI Prism 7700 software. The Cₜ value, an important raw data for each sample, was calculated as the cycle number at which the ΔRₙ crosses the baseline. For each run, a standard curve was constructed by plotting a graph with mean Cₜ values from 8 data points from standard sample against log input of the corresponding dilution values with a best fit trend line. From the trend line, the formula ‘y=mx+c’ was created according to the y-intercept and slope of standard curve which then were used for calculating the log input amount of the experimental cDNA samples, as related to the calibration sample. Data for the Novel PI-3-kinase adapter was normalized to that of beta-actin to correct for potential differences in efficiency of cDNA synthesis between the RNA samples.

From the TaqMan data the specificity to monocytes and macrophage found from the array data is confirmed and found to be even more pronounced (see FIG. 9b). The data presented in the Figure are listed below. In the data listed below, the normalized expression values are multiplied by 1000 for clarity.

| | | Normalized expression |
|---|---|---|
| Cell type | Oxygen | (clone p1E9/SeqID: 79/80) |
| adipocyte | normoxia | 0.050 |
| adipocyte | hypoxia 6 hr | 0.007 |
| adipocyte | hypoxia 18 hr | 0.015 |
| cardiomyocyte | normoxia | 0.163 |
| cardiomyocyte | hypoxia 6 hr | 0.037 |
| cardiomyocyte | hypoxia 18 hr | 0.222 |
| endothelial | normoxia | 3.093 |
| endothelial | hypoxia 6 hr | 0.059 |
| fibroblast | normoxia | 0.527 |
| fibroblast | hypoxia 6 hr | 0.043 |
| fibroblast | hypoxia 18 hr | 0.037 |
| macrophage | normoxia | 404.593 |
| macrophage | hypoxia 6 hr | 503.026 |
| macrophage | hypoxia 18 hr | 1162.056 |
| mammary epithelial | normoxia | 0.026 |
| mammary epithelial | hypoxia 6 hr | 0.068 |
| mammary epithelial | hypoxia 18 hr | 0.112 |
| monocyte | normoxia | 565.471 |
| monocyte | hypoxia 6 hr | 657.465 |
| monocyte | hypoxia 18 hr | 979.048 |
| neuroblastoma | normoxia | 8.482 |
| neuroblastoma | hypoxia 6 hr | 7.104 |
| neuroblastoma | hypoxia 18 hr | 4.707 |
| renal epithelial | normoxia | 17.898 |
| renal epithelial | hypoxia 6 hr | 9.831 |
| renal epithelial | hypoxia 18 hr | 10.929 |
| skeletal myocyte | normoxia | 0.930 |
| skeletal myocyte | hypoxia 6 hr | 0.638 |
| skeletal myocyte | hypoxia 18 hr | 1.627 |

There are several technical reasons why the results from the array-based data might be more pronounced in the Taqman results—the lower sensitivity of the array-based method means that genes which are not expressed will be detected as a background signal. Also the array method is more likely to suffer from cross-hybridization between similar genes.

The TaqMan data illustrates dramatically the concept that the hypoxia response is not just a generic response found in all cell types, relating to generic cell processes such as metabolism.

Database searches for gene sequences showing identity with IMAGE clone acc:R62339 reveal that there are no matching human sequences of any type other than ESTs. This includes full length cDNAs, truncated cDNAs, gene sequences from chromosomal data or hypothetical protein gene sequences. Therefore the human gene represented by IMAGE clone acc:R62339 is a novel human gene.

Although this human EST is unannotated, by comparison with mouse sequence data (acc AF293806), it appears likely to encode a novel human Phosphoinositol 3-kinase (PI3-kinase) adapter molecule, homologous to the recently described mouse gene, BCAP. The human gene is neither present on GenBank or published elsewhere. The probable mouse equivalent of this gene has been published and is a known gene sequence named originally by Okada and colleagues as “BCAP” (Okada T, Maeda A, Iwamatsu A, Gotoh K, Kurosaki T. “BCAP: the tyrosine kinase substrate that connects B cell receptor to phosphoinositide 3-kinase activation”. Immunity. December 2000;13(6):817-27) (GenBank accession: NM_{—}031376). Briefly this and subsequent papers by the same authors indicate that BCAP is only relevant in B-cells.

Protein components of the Phosphoinositol 3-kinase (PI3-kinase) signalling cascade, involved in intracellular signalling, have been implicated clearly as potential drug targets (see Stein R C et al, “PI3-kinase inhibition: a target for drug development” Mol Med Today. September 2000;6(9):347-57). P13-kinases are a ubiquitously expressed enzyme family and, through the generation of phospholipid second messengers, are key to many cellular processes relevant to human disease, including proliferation, apoptosis and inflammation, motility, carbohydrate metabolism and intracellular protein sorting. These molecules are activated by receptor tyrosine kinases, src-like tyrosine kinases and viral oncoproteins. Studies of mutants that abrogate the binding of P13-kinases to these molecules has indicated that P13-kinases mediate mitogenic and cell motility responses of cells to growth factors and oncoproteins. As knowledge of their involvement in disease processes increases, the PI3-kinases and other components of the P13-kinase signalling cascade appear to be an increasingly attractive target for drug development, particularly in the fields of cancer and other proliferative diseases, and in the treatment of inflammatory and immunological conditions. Evidence of the functional specialization of PI3-kinase isoforms suggests that selective inhibition with acceptable toxicity might be possible. Furthermore, there is evidence from mouse studies that functional ablation of BCAP is likely to have clinically significant effect on inflammatory disease (Yamazaki et al 2002, J Exp Med 195(5):535-45).

The data presented for the gene encoded by SEQ ID NO::79/80 thus provides evidence that the encoded protein is a novel drug target in humans, specifically targeting monocyte/macrophages at hypoxic disease sites.

In the publication relating to murine BCAP, the protein is identified as an adapter molecule connecting the non-receptor protein tyrosine kinase Syk to the p85 subunit of P13-kinase, and therefore to the pivotal signalling pathways centred around P13-kinase (Okada T et al “BCAP: the tyrosine kinase substrate that connects B cell receptor to phosphoinositide 3-kinase activation.” Immunity. 2000 13:817-27). Although, in this report, Syk is acting as the intracellular signalling component of the B cell antigen receptor, which is present exclusively on B-cells, Syk has been shown to initiate intracellular signalling from other cell surface receptors which are expressed on macrophages, including the Fc gamma receptor, the chemokine receptor CCR5 and macrophage-expressed CD8 (Darby C et al “Stimulation of macrophage Fc gamma RIIIA activates the receptor-associated protein tyrosine kinase Syk and induces phosphorylation of multiple proteins including p95Vav and p62/GAP-associated protein”. J Immunol. 1994 152:5429-37) (Kedzierska K et al “FcgammaR-mediated phagocytosis by human macrophages involves Hck, Syk, and Pyk2 and is augmented by GM-CSF.” J Leukoc Biol. August 2001;70(2):322-8.), (Ganju R K et al “Beta-chemokine receptor CCR5 signals through SHP1, SHP2, and Syk.” J Biol Chem. 2000 275:17263-8.), (Lin T J et al “Activation of macrophage CD8: pharmacological studies of TNF and IL-1 beta production.” J Immunol. 2000 164:1783-92.).

Indeed, syk has been validated as target in macrophages to inhibit inflammatory activities of this cell type (Stenton G R et al “Aerosolized Syk antisense suppresses Syk expression, mediator release from macrophages, and pulmonary inflammation.” J Immunol. Apr. 1, 2000;164(7):3790-7.).

By screening a cDNA library derived from human peripheral blood mononuclear cells, two cDNA clones corresponding to BCAP have been obtained [clone GLB_{—}3 and clone GLB_{—}4]. Rapid Amplification of cDNA Ends (RACE) has also been performed to extend these sequences.

A detailed analysis of this data has been performed, and compared to the recent prediction of the human BCAP gene made by the human genome Ensembl project (www.ensembl.org/) (Ensembl predicted transcript accession ENST00000286061). The Ensembl gene prediction is equivalent in structure to the mouse sequence (NM_{—}031376), though it is truncated at the 5′ end compared to the mouse sequence.

Analysis of the sequence data indicates that the human BCAP gene is composed of 19 exons, 3 of which have no matching ESTs in GenBank and are not predicted by Ensembl (exons 1,3 and 6). Furthermore we show that alternative splicing leads to alternative isoforms which encode discrete polypeptide sequences.

The isoform of human BCAP mRNA which is equivalent to ENST00000286061 and mouse BCAP, we now call Hu.BCAP-A (encoding 805aa) and have experimentally determined its full and accurate sequence. Based on the exon structure and nomenclature, Hu.BCAP-A is comprised of a combination of exons 1 [SEQ ID NO:490], 2 [SEQ ID NO:492], 3, [SEQ ID NO:494], 4 [SEQ ID NO:496], 5 [SEQ ID NO:498], 6 [SEQ ID NO:500], 7 [SEQ ID NO:502], 8 [SEQ ID NO:504], 9 [SEQ ID NO:506], 10 [SEQ ID NO:508], 11 [SEQ ID NO:510], 12 [SEQ ID NO:512], 13 [SEQ ID NO:514], 14 [SEQ ID NO:516], 15 [SEQ ID NO:518], 16 [SEQ ID NO:520], 17 [SEQ ID NO:522], 18 [SEQ ID NO:524] and 19 [SEQ ID NO:526]. The full Hu.BCAP-A nucleotide sequence is presented in SEQ ID NO:528. The human gene has clear utility in the design of therapeutics for the treatment of human disease. The fuill Hu.BCAP-A polypeptide sequence is presented in SEQ ID NO:527.

Our cDNA sequencing work indicated that an alternative transcript which we now call BCAP-B is formed by the splicing pattern of exons: 6, 7, 8, 9, 10, 11, 12, 13, 14, 15, 16, 17, 18, 19. This encodes a protein of 540aa which is a truncated version of BCAP-A (SEQ ID NO: 529, encoded by SEQ ID NO:530). This truncation in the encoded protein is not due to inadequate sequencing, but reflects a different exon usage. Novel functions and utilities are expected for BCAP-B, compared to BCAP-A and mouse BCAP.

Our cDNA sequencing work indicated that a further alternative transcript which we now call BCAP-C is formed by the splicing pattern of exons: 1, 2, 3, 4, 7, 8, 9, 10, 11, 12, 13, 14, 15, 16, 17, 18, 19. This encodes a228aa polypeptide which as well as being shorter than BCAP-A also contains novel amino acid sequence derived from the novel exon 3 (SEQ ID NO:531, encoded by SEQ ID NO:532). Furthermore, as a result of exon 4 being directly spliced to exon 7 (rather than to exon 5 as found in BCAP-A) a frameshift is introduced and a small tail of 8 residues is created prior to reaching the first stop codon of this new frame. Again BCAP-C is expected to have novel functions and utilities.

Additional to the finding that the probable human orthologue of the adapter molecule BCAP is preferentially hypoxia-induced in human monocytes/macrophages, we also find from data generated by the custom array, that the protein acting immediately upstream of BCAP (i.e. Syk) is also regulated by hypoxia in this novel cell type specific manner, greatly increasing the biological significance of the original finding (see FIG. 9c). The data used to generate this Figure are presented below for clarity.

| | | Normalized expression |
|---|---|---|
| Cell type | Oxygen | (of syk) |
| adipocyte | normoxia | 2.6573591 |
| adipocyte | hypoxia 6 hr | 1.499927 |
| adipocyte | hypoxia 18 hr | 1.1115488 |
| cardiomyocyte | normoxia | 0.8357341 |
| cardiomyocyte | hypoxia 6 hr | 2.161058 |
| cardiomyocyte | hypoxia 18 hr | 0.90880114 |
| endothelial | normoxia | 0.60265505 |
| endothelial | hypoxia 6 hr | 0.56874704 |
| endothelial | hypoxia 18 hr | 0.43321633 |
| fibroblast | normoxia | 0.8542026 |
| fibroblast | hypoxia 6 hr | 0.7657573 |
| fibroblast | hypoxia 18 hr | 0.784982 |
| hepatocyte | normoxia | 0.5238476 |
| hepatocyte | hypoxia 6 hr | 0.8465495 |
| macrophage | normoxia | 4.272981 |
| macrophage | hypoxia 6 hr | 6.144931 |
| macrophage | hypoxia 18 hr | 10.278416 |
| mammary epithelial | normoxia | 1.1023632 |
| mammary epithelial | hypoxia 6 hr | 2.7382789 |
| mammary epithelial | hypoxia 18 hr | 0.7985004 |
| monocyte | normoxia | 6.052118 |
| monocyte | hypoxia 6 hr | 8.6809225 |
| monocyte | hypoxia 18 hr | 11.58468 |
| neuroblastoma | normoxia | 1.0230793 |
| neuroblastoma | hypoxia 6 hr | 1.089154 |
| neuroblastoma | hypoxia 18 hr | 0.7689335 |
| renal epithelial | normoxia | 0.88565326 |
| renal epithelial | hypoxia 6 hr | 1.2609364 |
| renal epithelial | hypoxia 18 hr | 0.6242461 |
| skeletal myocyte | normoxia | 1.3959162 |
| skeletal myocyte | hypoxia 6 hr | 0.91255134 |
| skeletal myocyte | hypoxia 18 hr | 0.64795935 |

In summary, we have shown here that a novel human gene encoding a predicted signalling protein relevant to human disease is activated by hypoxia, specifically in monocytes and macrophages. This data is validated by non-array based means. Furthermore, we identify the protein immediately upstream of this signalling system as being co-regulated in this manner too. Therefore the human PI3-kinase adapter encoded by IMAGE clone acc: R62339 and the non-receptor tyrosine kinase Syk are both identified here for the first time as therapeutic targets for diseases involving hypoxic macrophages, including Rheumatoid arthritis, chronic occlusive pulmonary disease, atherosclerosis and cancer. Because both genes are preferentially expressed in hypoxic macrophages, toxicity effects of therapeutic products directed at the encoded proteins are likely to be limited.

As discussed in detail above, fragments and functional equivalents of the PI-3-kinase adapter protein represented in SEQ ID NO::79/80 and other equivalent proteins are included within the present invention, in addition to ligands that bind specifically to these proteins. Furthermore, the invention also embraces purified and isolated nucleic acid molecules encoding these proteins, fragments and functional equivalents, vectors containing such nucleic acid molecules and host cells transformed with these vectors.

Regulator of G-protein Signalling 1 (SEQ ID NO::375/376)

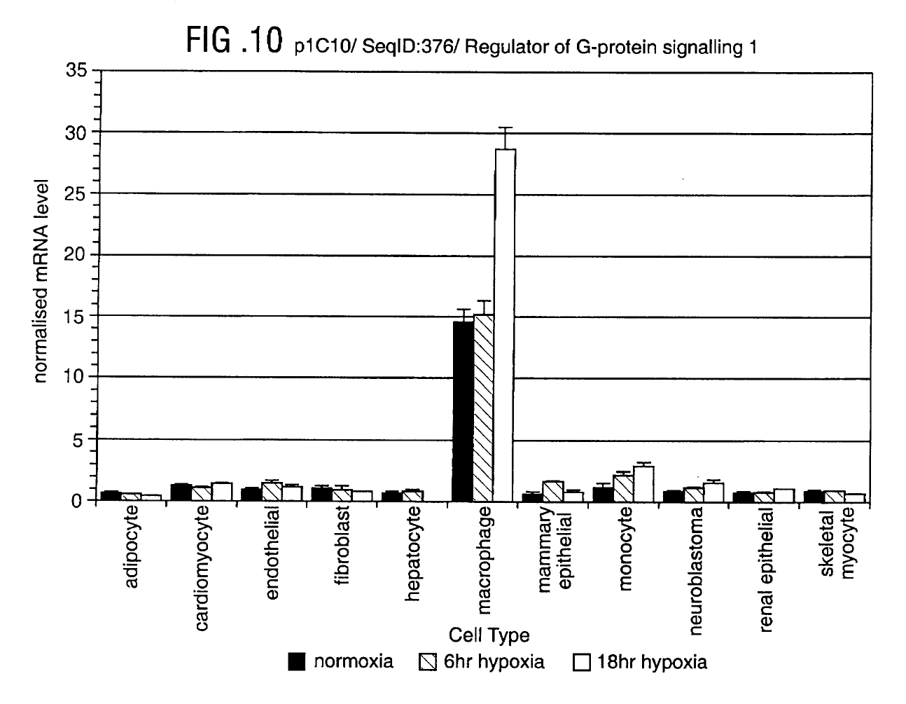

Another intracellular signalling protein, Regulator of G-protein signalling 1 (RGS1; SEQ ID NO::375/376), in shown in FIG. 10. Here the expression levels in the hypoxic monocyte is 30-fold higher than the median expression level of this gene throughout the other cell types. The function of this protein is to negatively regulate G protein signalling pathways, and inhibit chemokine-induced cell migration of immune cells (Moratz C et al J Immunol. 2000 164:1829-38 and Denecke B et al J Biol Chem. 1999 274:26860-8.).

Our data suggests that this gene is preferentially expressed in macrophages, consistent with the findings of Denecke B et al (J Biol Chem. 1999 274:26860-8.). Our novel finding that expression is even further enhanced by hypoxia illuminates a mechanism by which cell migration is inhibited in hypoxia, leading to an accumulation of these cells at pathological sites of hypoxia. This mechanism is novel and distinct to other mechanisms proposed in the art to explain this key aspect of hypoxia and inflammation (for example: Grimshaw M J et al “Inhibition of monocyte and macrophage chemotaxis by hypoxia and inflammation—a potential mechanism.” Eur J Immunol. 2001 31:480-9).

Furthermore, FIG. 10 shows that Regulator of G-protein signalling 1 is upregulated during differentiation of monocytes to macrophages, with significance to changes in cell motility. This discovery therefore provides that inhibitors of RGS1 have utility in increasing the motility of macrophages that are used for cell-based therapies. Accordingly, one embodiment of this aspect of the invention provides for the use of an inhibitor of RGS1 in therapy, by increasing the motility of macrophage cells.

GM2 Ganglioside Activator Protein

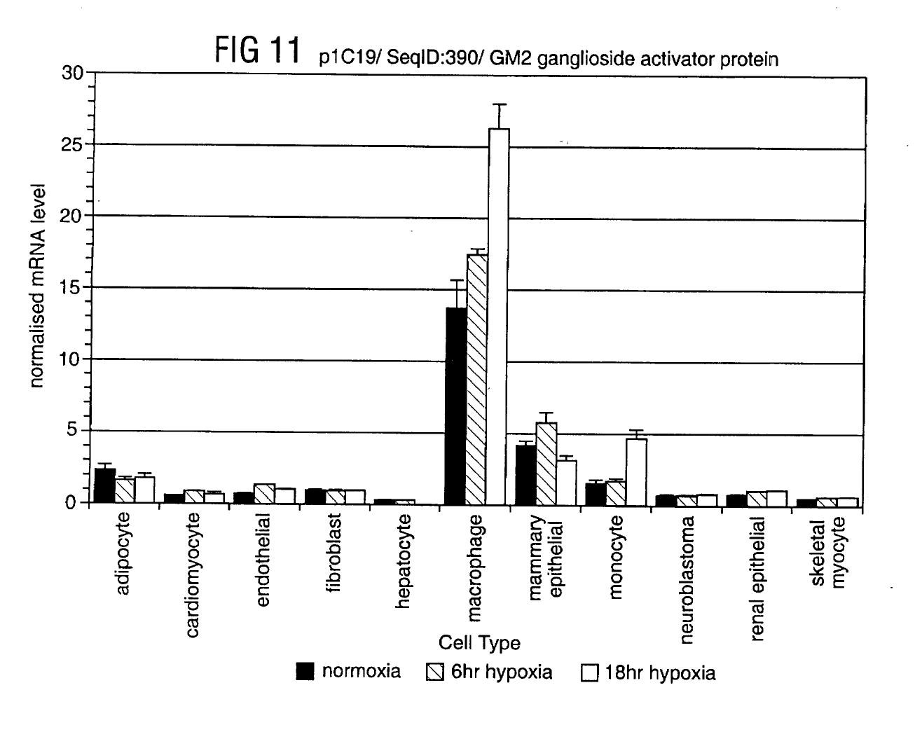

The gene shown in FIG. 11, GM2 ganglioside activator protein, was originally characterized as a lysosomal co-factor required for degradation of gangliosides. It has been proposed to have alternative roles as a secreted protein, and can bind and inhibit the actions of the inflammatory mediator, platelet activating factor (Rigat B et al Biochem Biophys Res Commun. 1999 258:256-9.).

Our novel finding, presented in FIG. 11, shows that GM2 ganglioside activator protein is induced by hypoxia, preferentially in macrophages, suggesting an influence on the inflammatory functions of the macrophage in hypoxia.

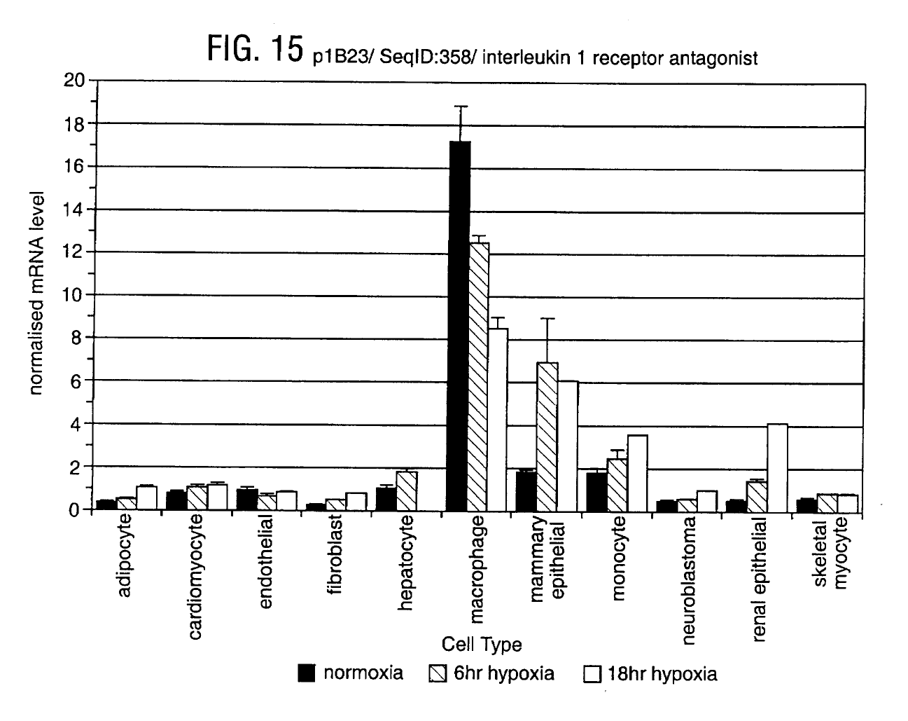

In FIGS. 15-18, genes are shown which are expressed preferentially in the monocyte/macrophage, but which are decreased in expression in response to hypoxia. Being expressed at highest levels in the monocyte/macrophage, these genes are more likely to be significant to the biological functions of this cell type.

Interleukin 1 Receptor Antagonist (SEQ ID NO::357/358)

In FIG. 15, the gene interleukin 1 receptor antagonist (SEQ ID NO::357/358) is seen to be down-regulated by hypoxia in the macrophage. Since the function of the encoded protein is anti-inflammatory, then down-regulation of this gene would be expected to have a pro-inflammatory effect. Therefore, corrective expression of the gene, would be expected to produce therapeutic effects in inflammatory disorders involving macrophages and hypoxia, such as Rheumatoid Arthritis (Hollander A P et al. Arthritis Rheum. 2001 44:1540-4). This correlates with effects seen from the application the drug Anakrina/Kineret™ developed by Amgen. This supports the applicability of the genes disclosed herein as novel targets for therapeutic products.

The example of gene interleukin 1 receptor antagonist also provides good exemplification of the concept that different cell types respond to hypoxia differently. Here, not only are there quantitative differences, but also qualitative differences in that this gene is down-regulated by hypoxia in macrophages, but up-regulated by hypoxia in several other cell types, such as renal epithelial cells (see FIG. 15). Such findings are not documented in the art.

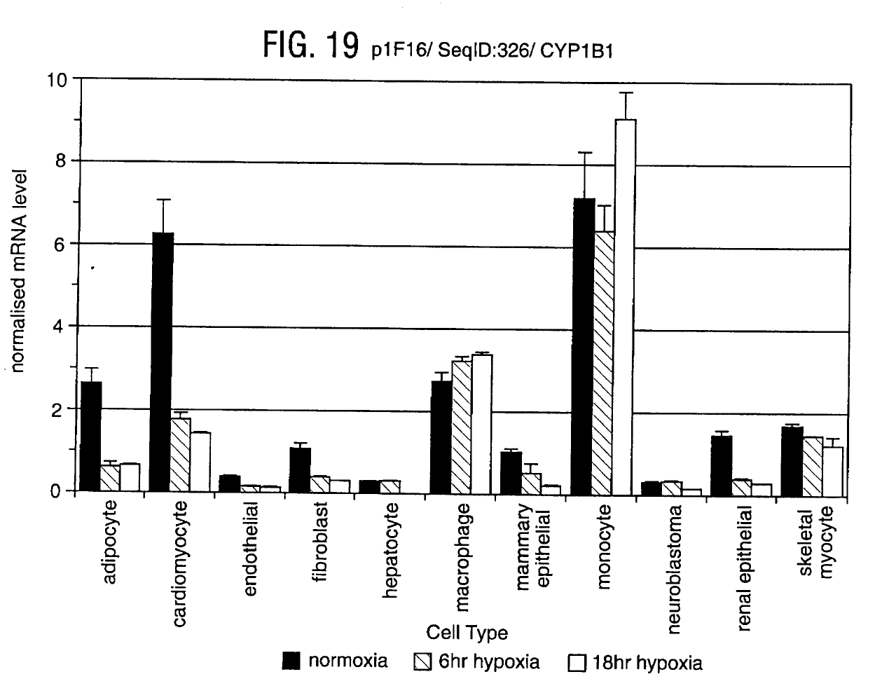

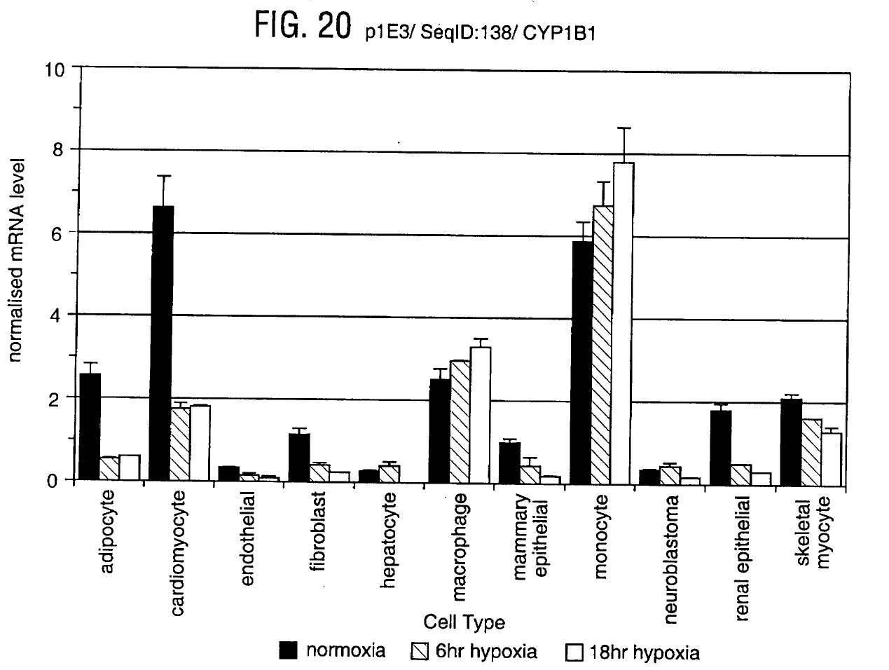

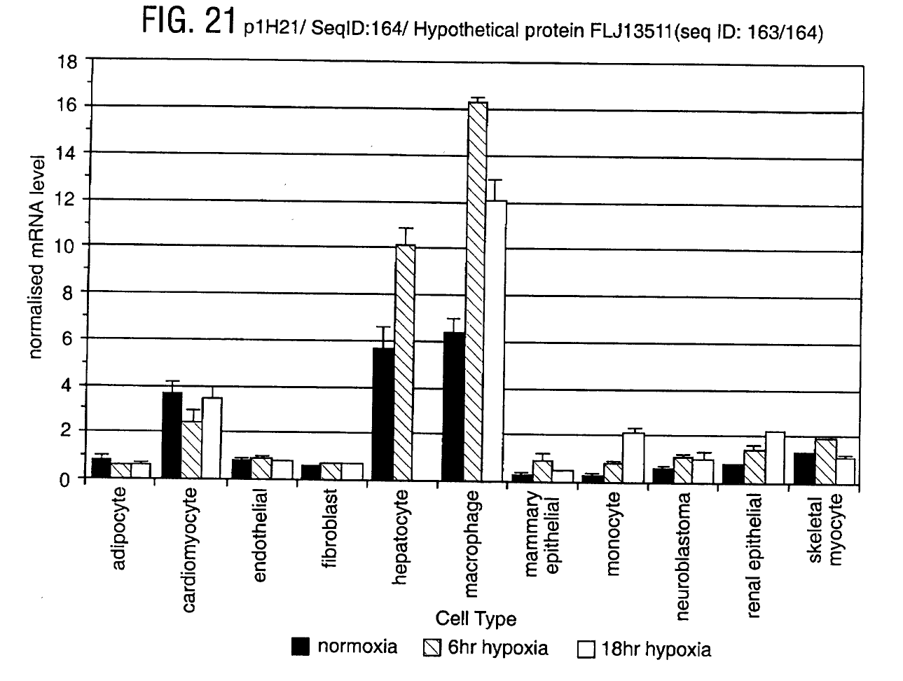

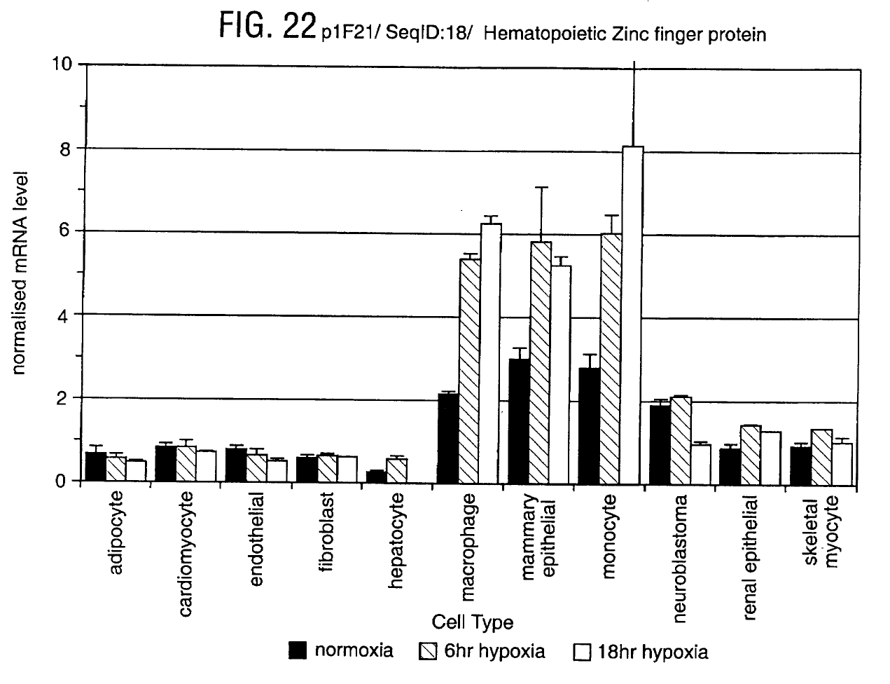

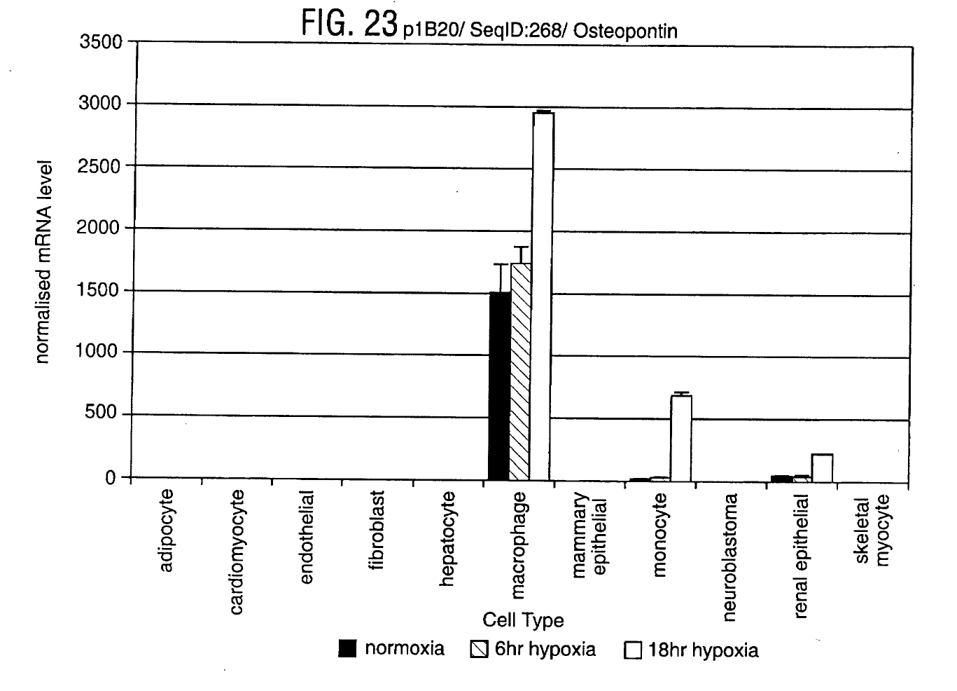

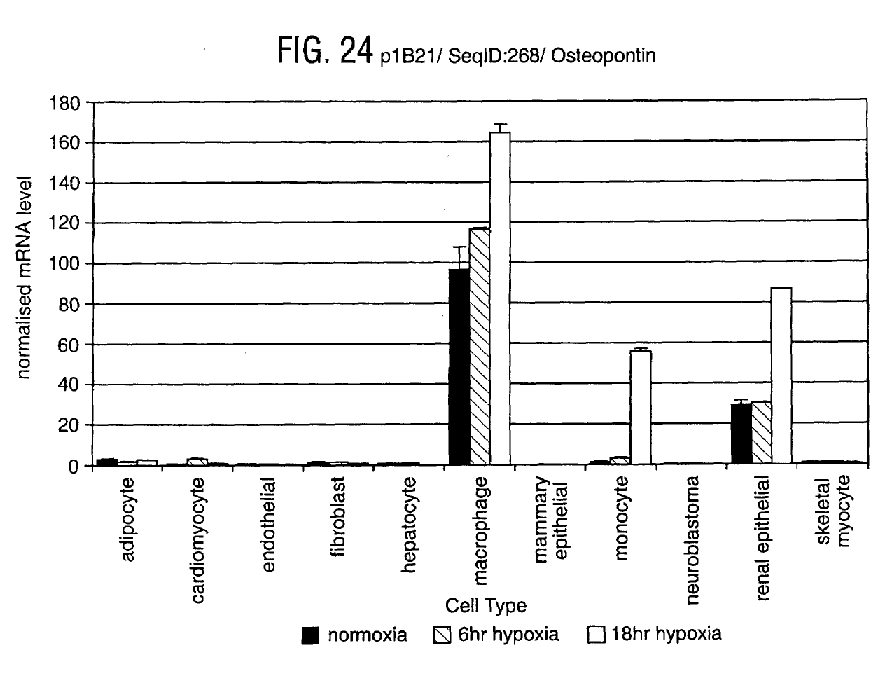

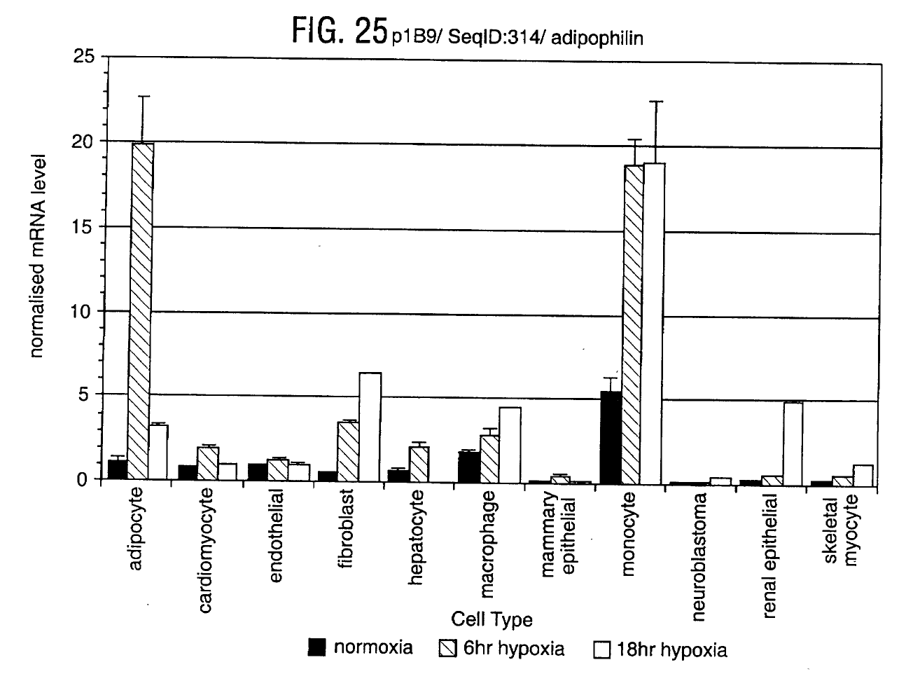

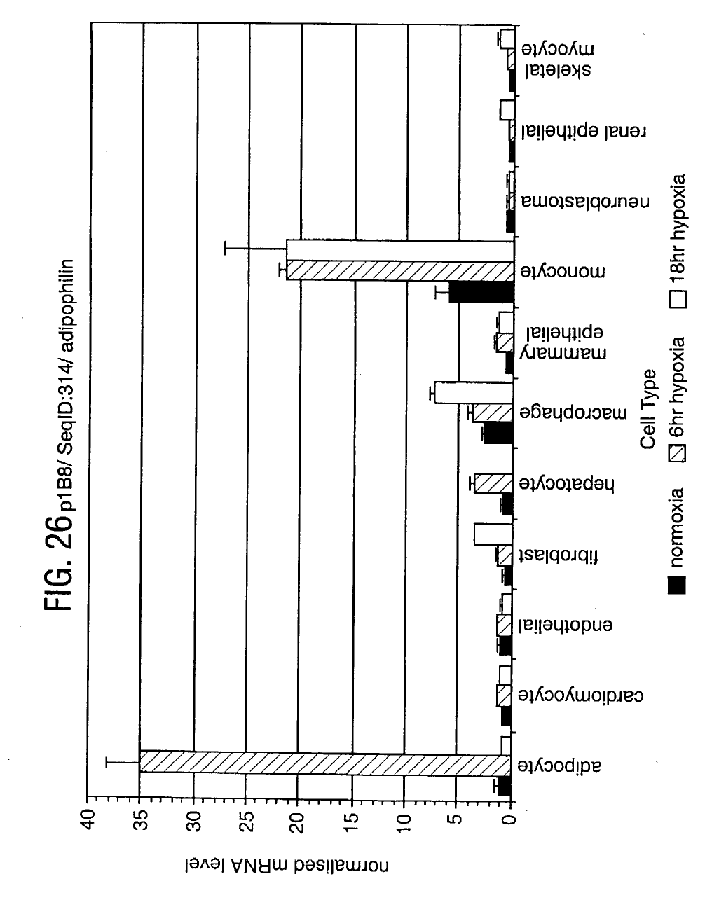

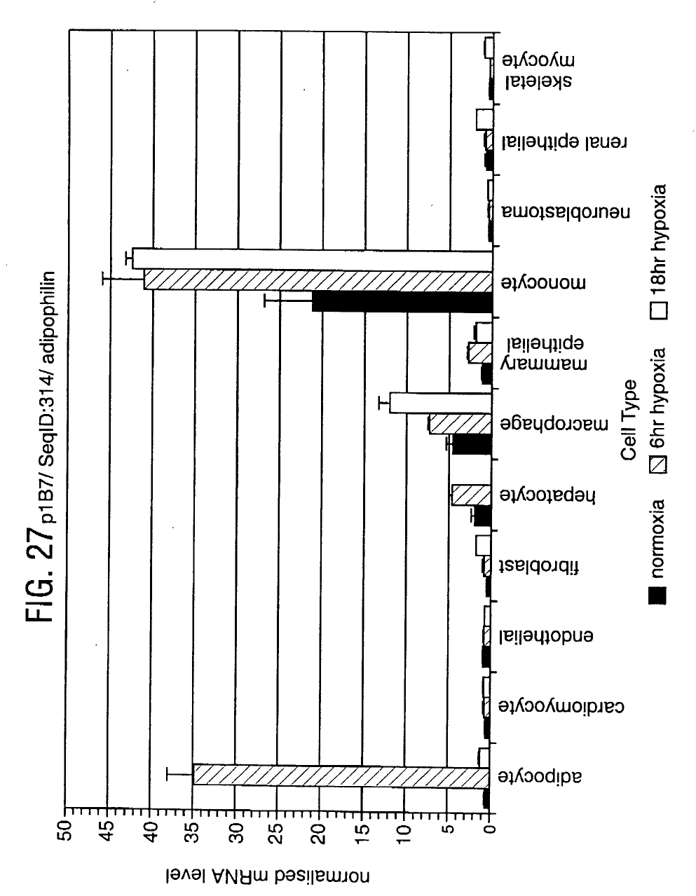

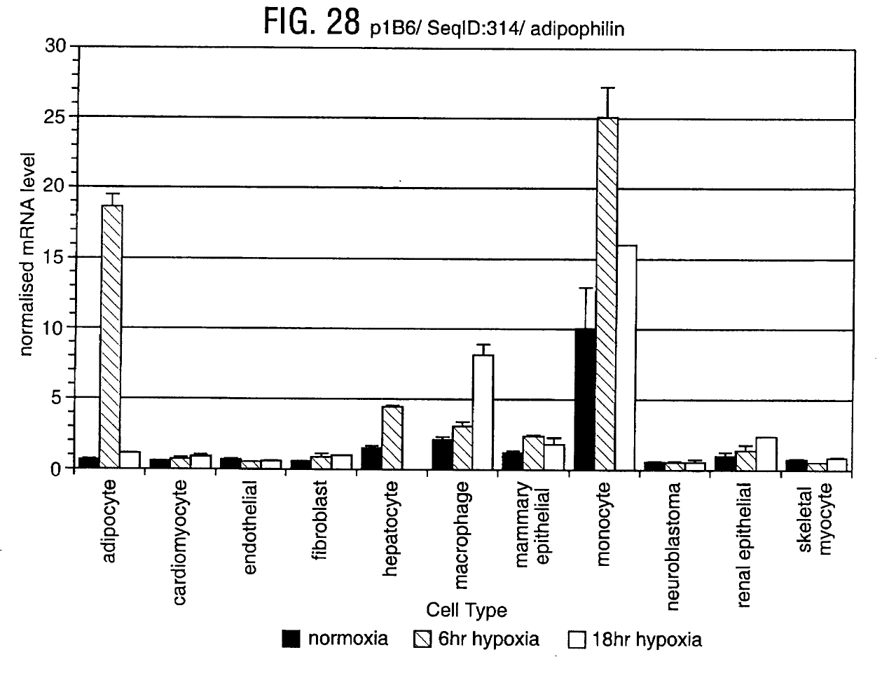

The dataset of Table 12 also contains genes which are induced preferentially in monocyte/macrophages and also in some but not all other cell types tested. Several of these genes are present as multiple clones on the gene array, giving separate data, therefore adding extra confidence to the conclusions. These genes, presented in FIGS. 19-28 correspond to:

SeqID:313/314 adipophilin

SeqID:163/164 hypothetical protein FLJ13511

SeqID:267/268 Osteopontin

SeqID:17/18 Hematopoietic Zinc finger protein

SeqID:137/138 CYP1B1

SeqID:325/326CYP1B1

It will also be seen that in the case of CYP1B1 (clones p1F16 and p1E3) the hypoxia response in monocytes/macrophages is qualitatively different to the other cell types tested, in that the gene is up-regulated rather than down-regulated in response to hypoxia.

Genes with a Greater Response in Endothelial Cells

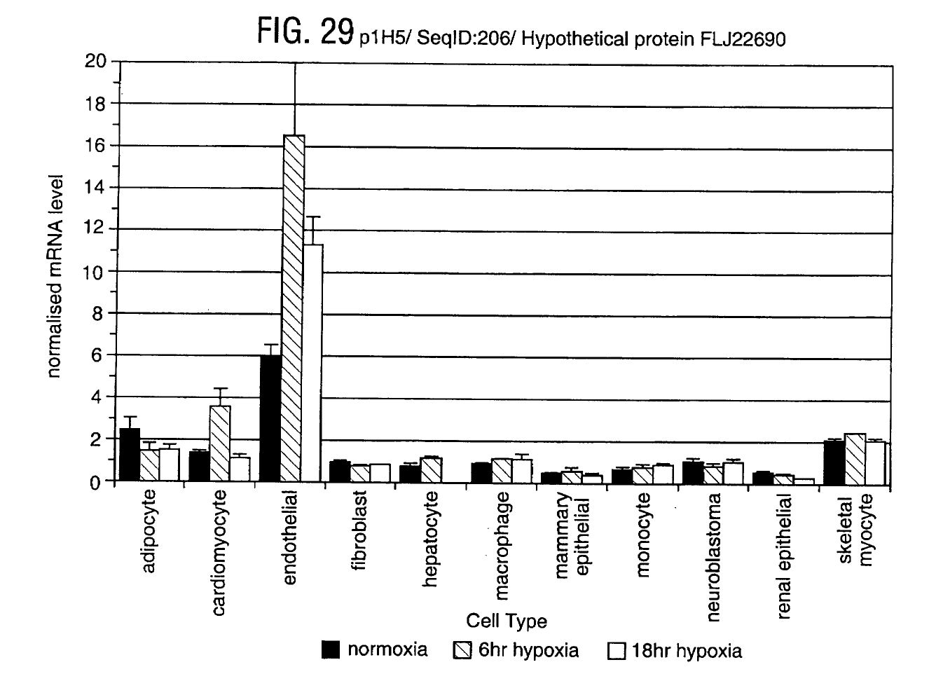

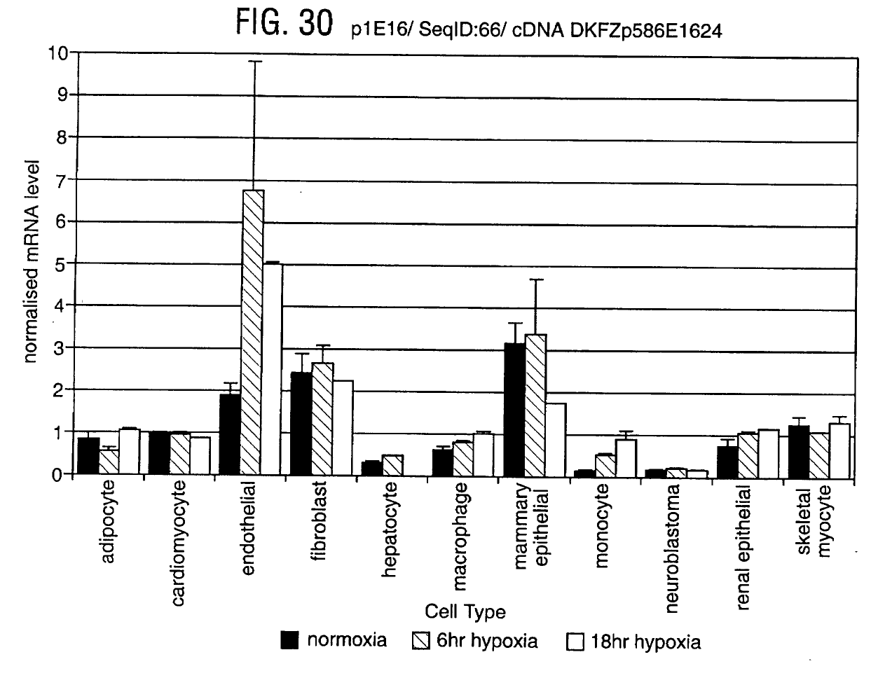

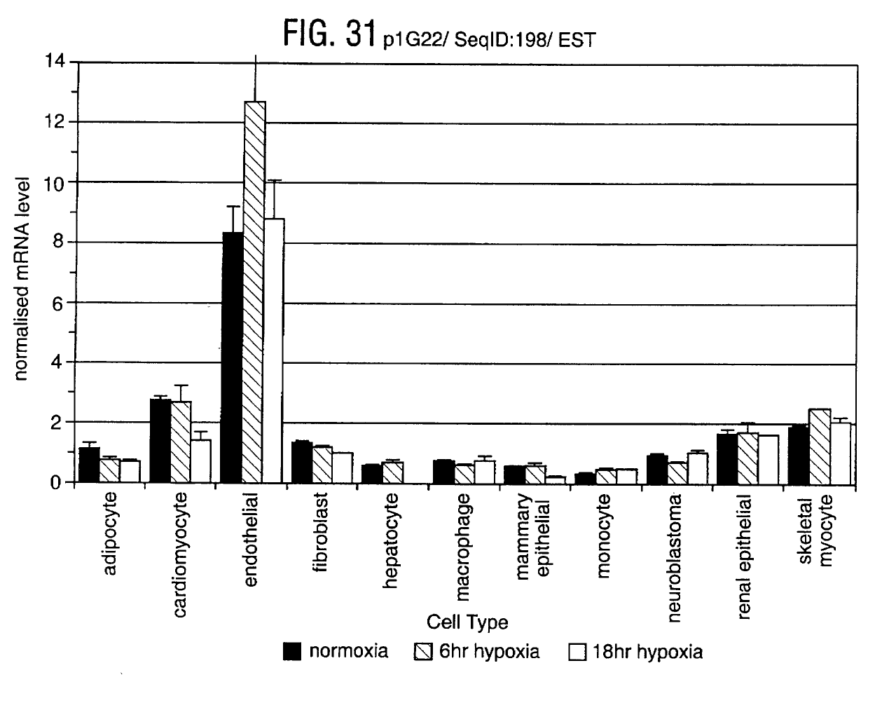

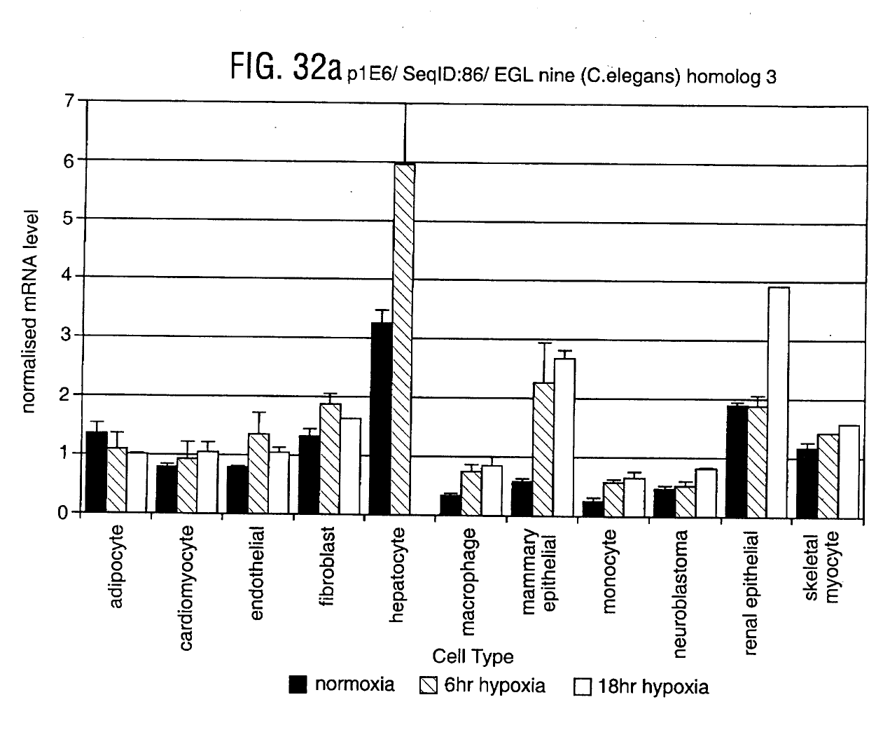

The dataset of Table 12 also contains genes which are induced preferentially in endothelial cells, a cell type key to the process of angiogenesis, in response to hypoxia. These genes are as follows, and are presented in FIGS. 29-31:

SeqID:205/206 hypothetical protein FLJ22690

SeqID:65/66 cDNA DKFZp586E1624

SeqID:197/198 EST

Genes with a Greater Response in Hepatocytes

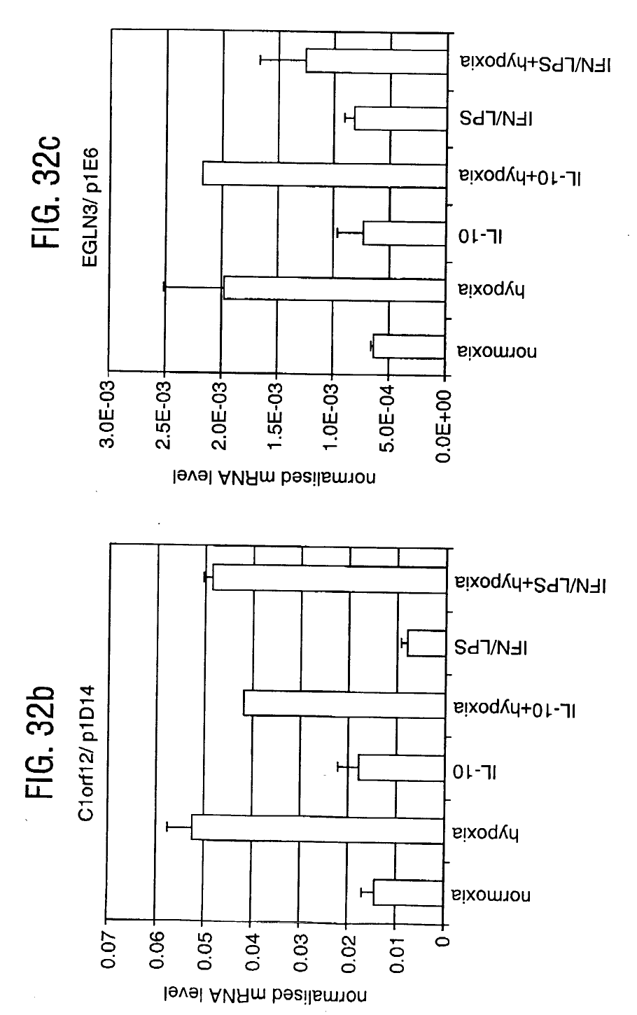

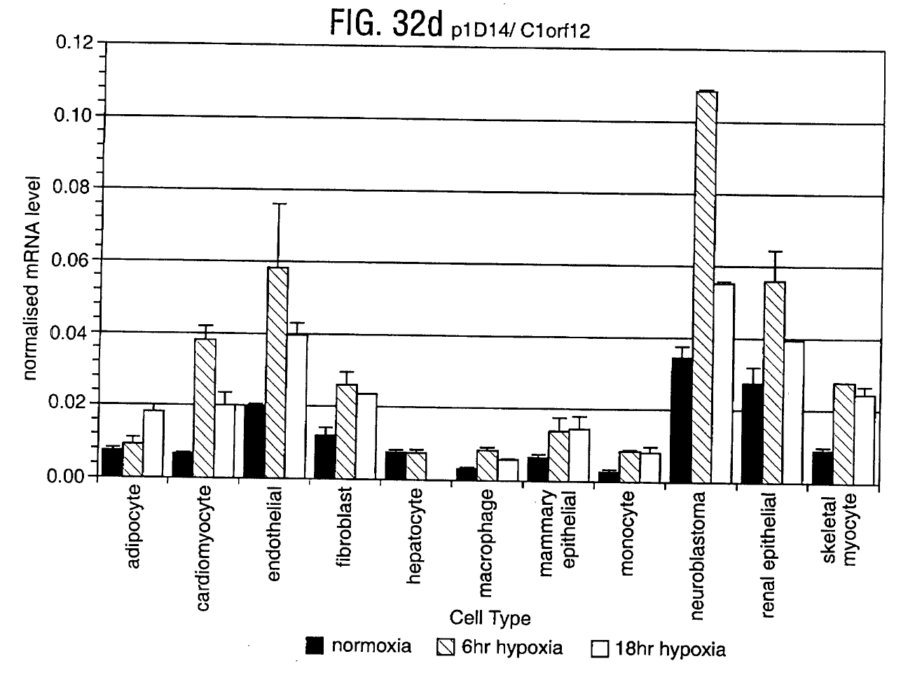

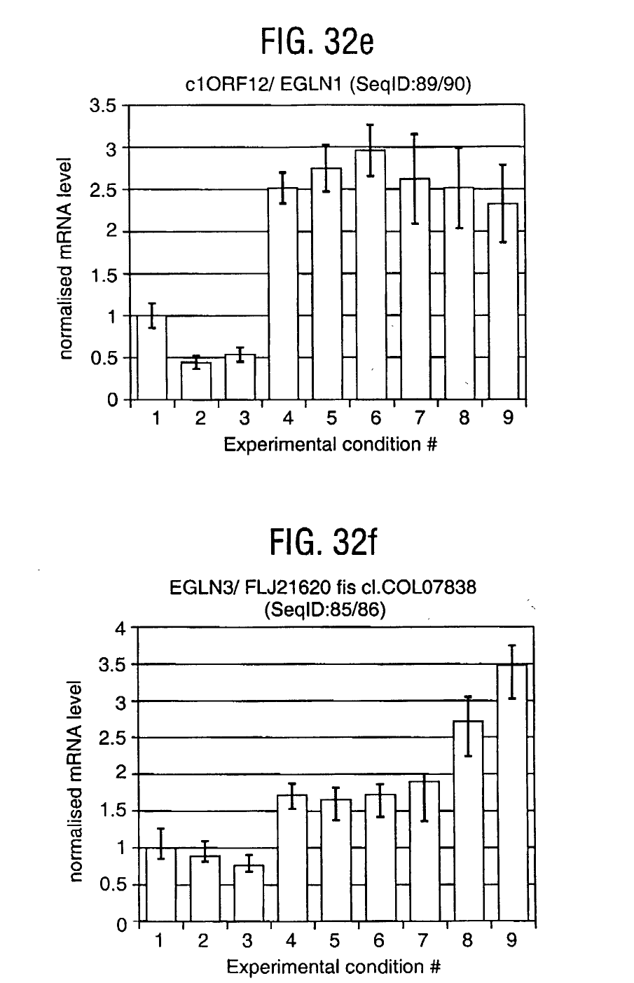

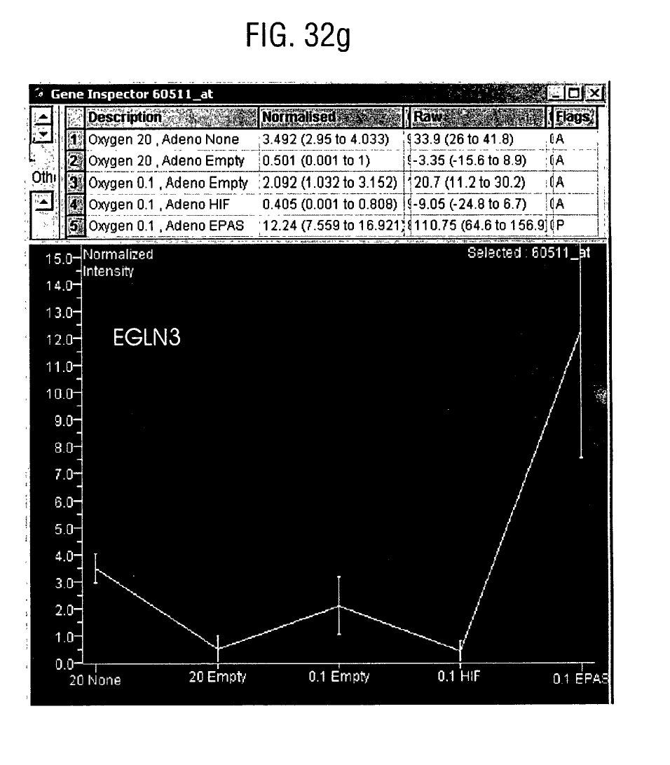

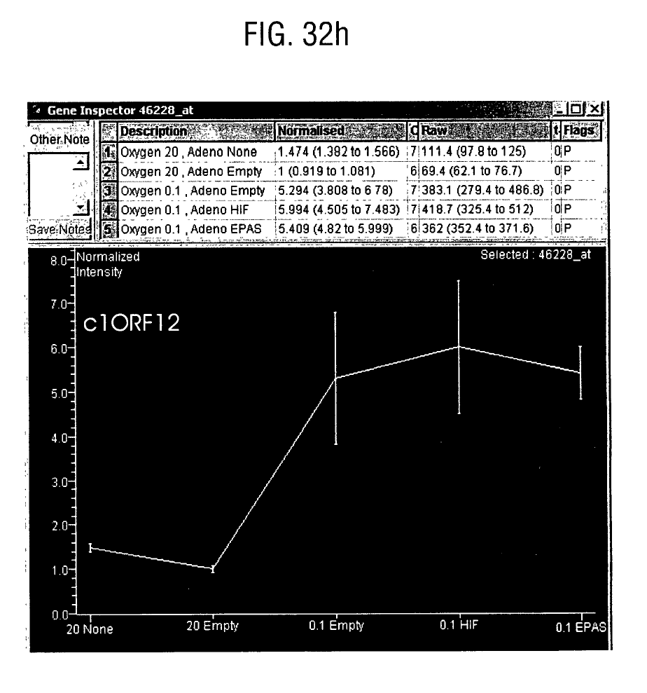

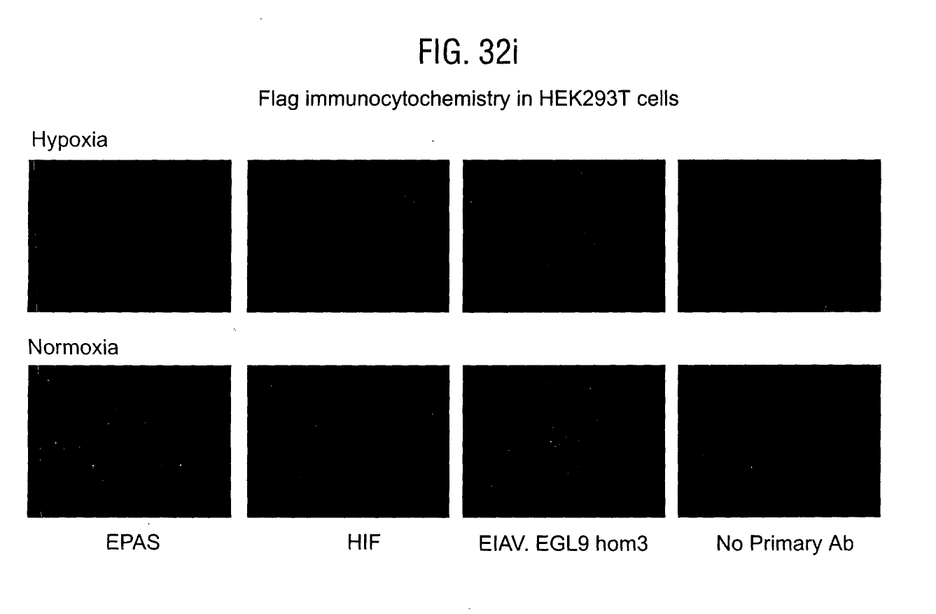

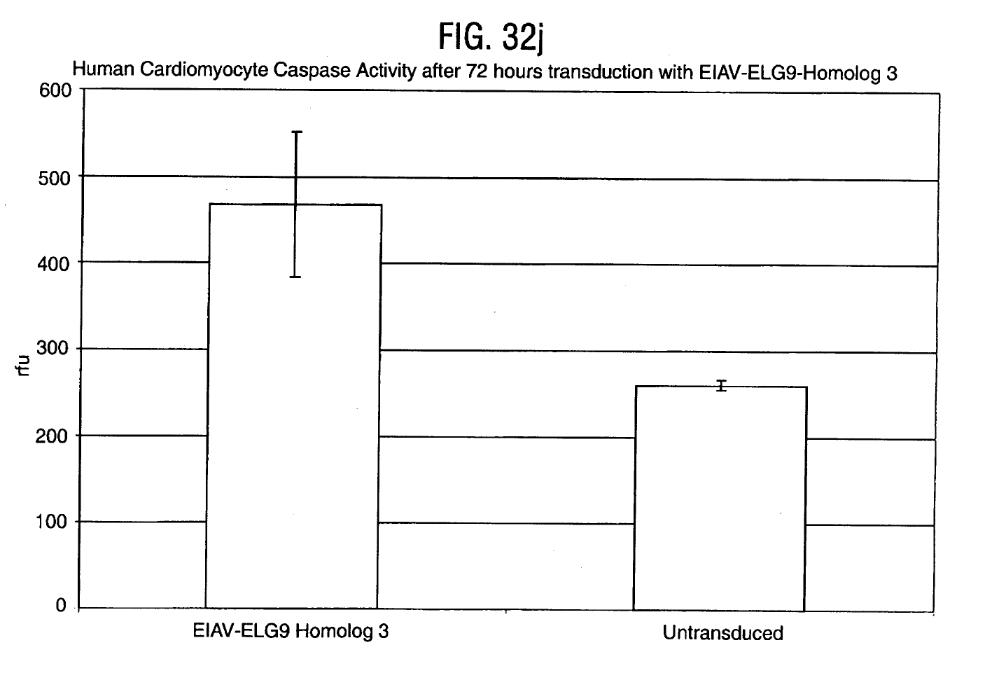

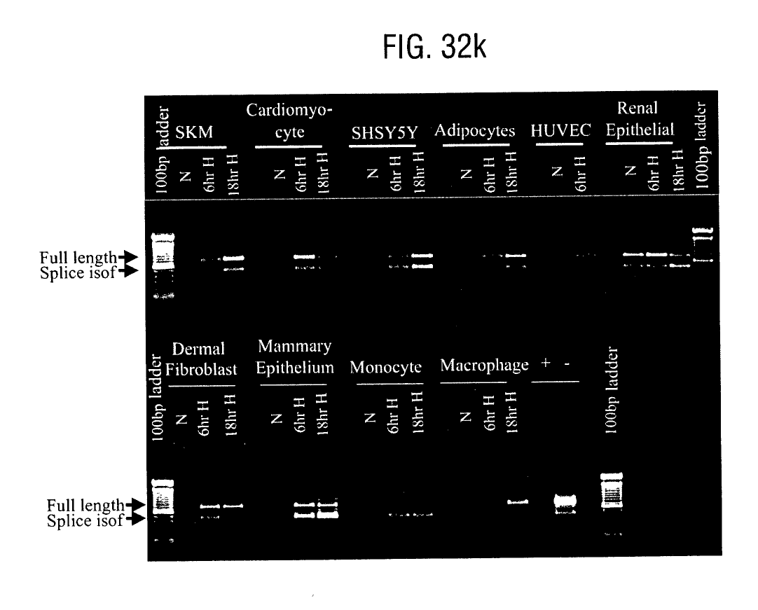

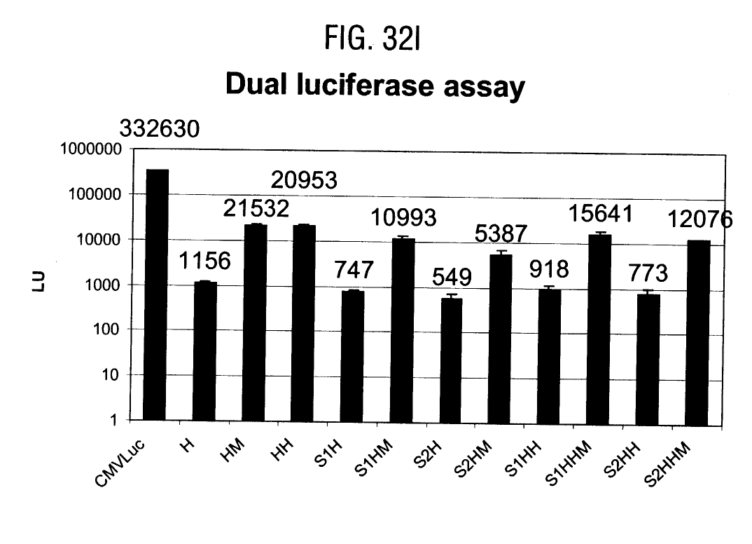

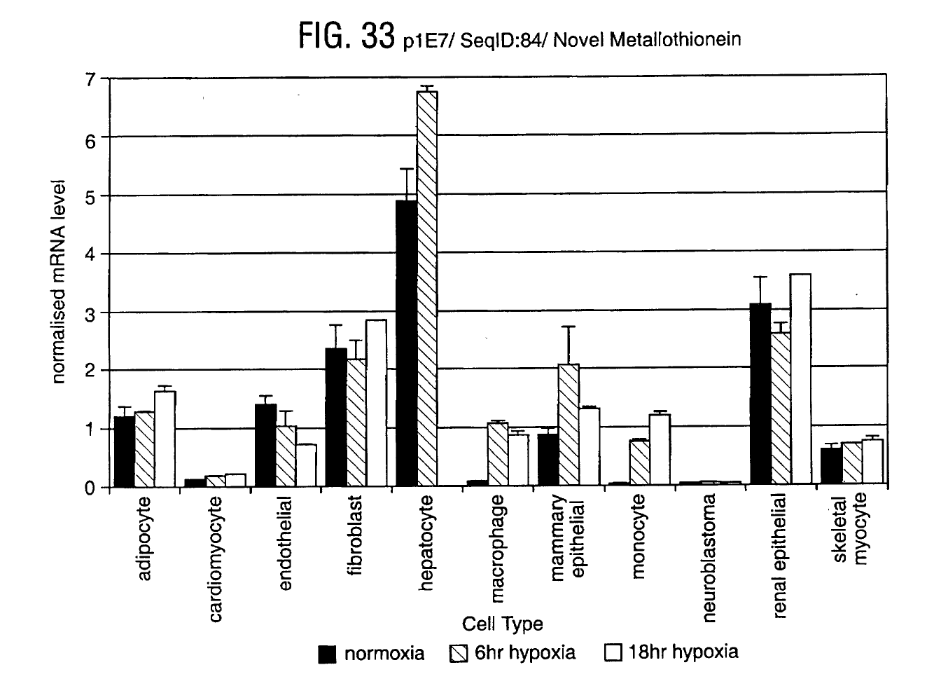

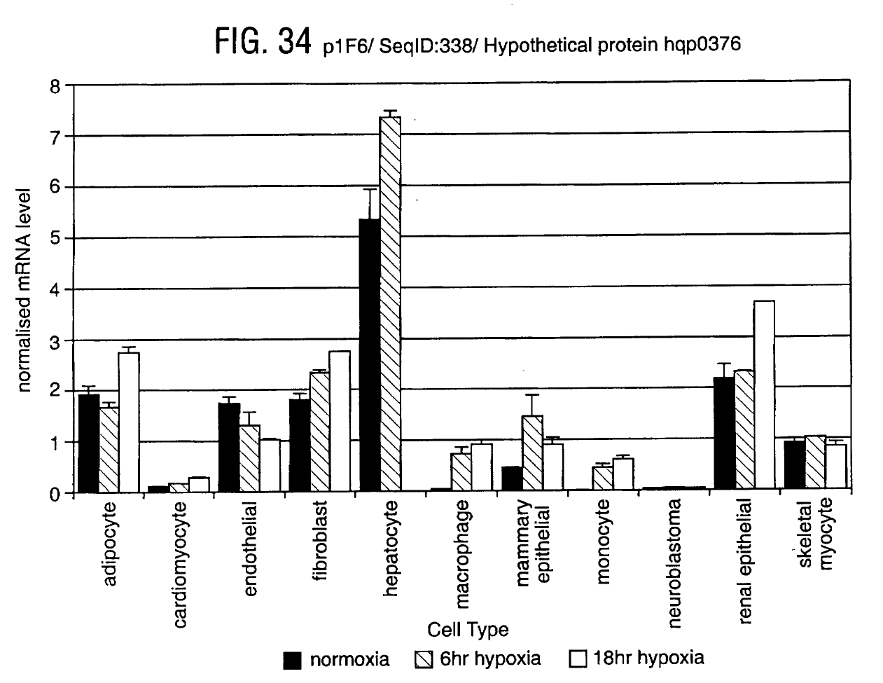

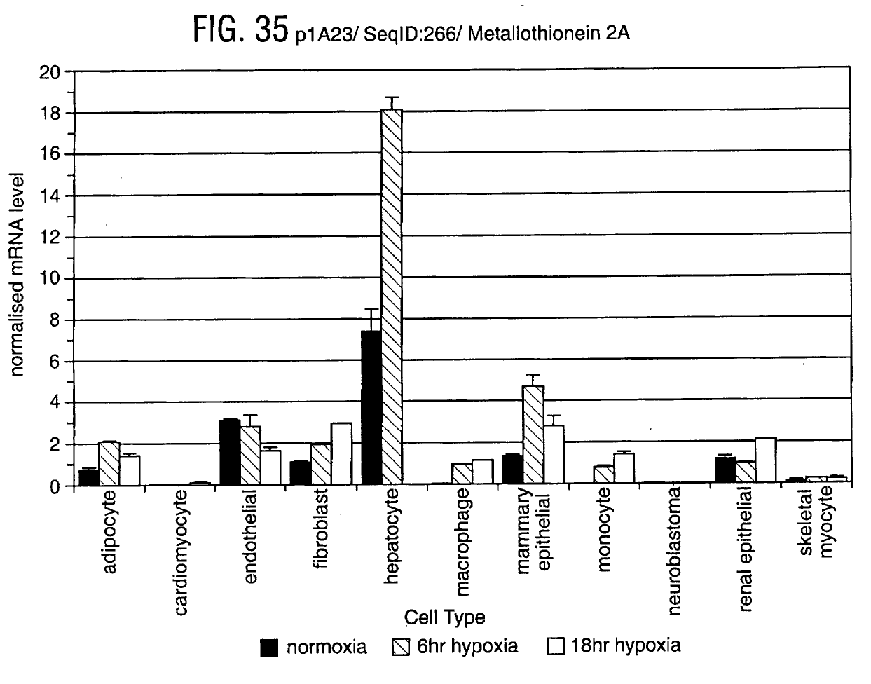

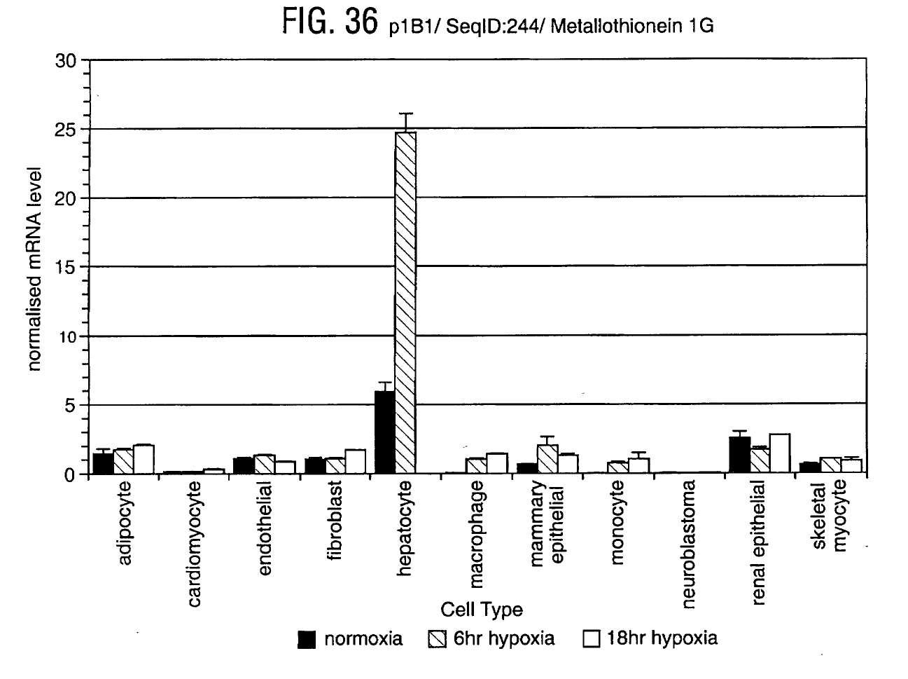

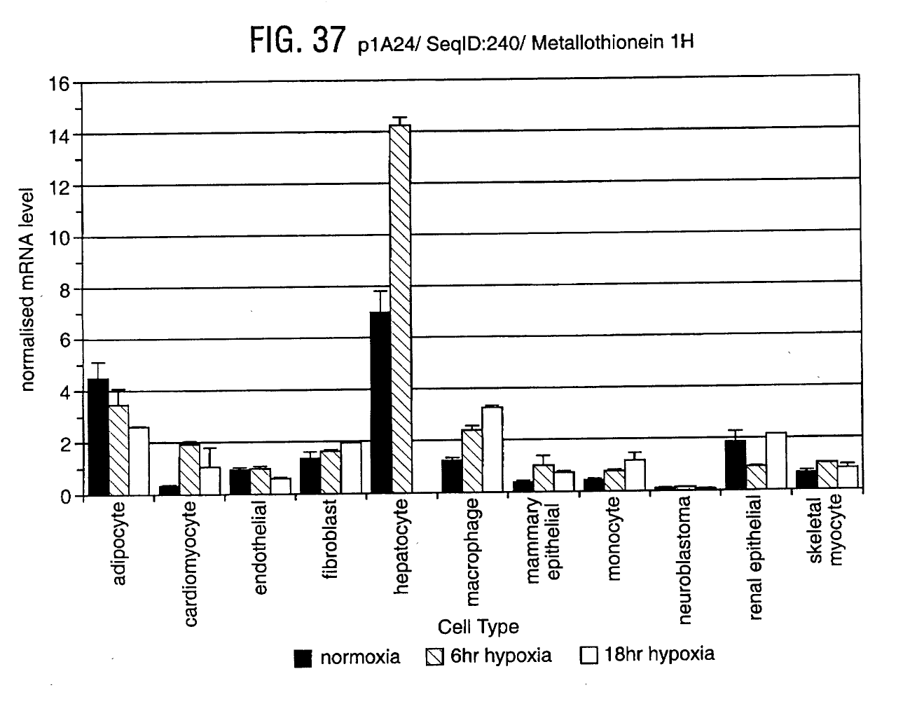

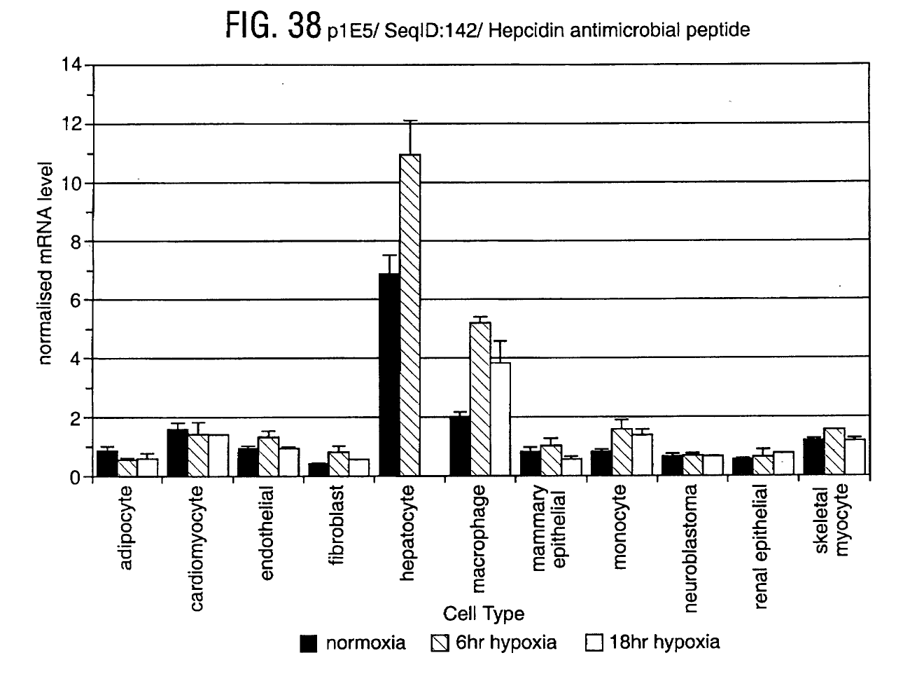

The dataset of Table 12 also contains genes which are induced preferentially in hepatocytes, in response to hypoxia. These genes are presented in FIGS. 32a and 33-38. It is noted that most of these genes, including hqp0376, encode proteins of the metallotheionein family. Furthermore, close inspection of these data reveals that the fold induction in hypoxia compared to normoxia for monocyte/macrophages are very high, though the absolute levels of expression are below that of hepatocytes.

SeqID:85/86 EGL nine (C.elegans) homolog 3

SeqID:83/84 Novel Metallothionein

SeqID:337/338 hypothetical protein hqp0376 (a metallotheionein)

SeqID:265/266 Metallothionein 2A

SeqID:243/244 Metallothionein 1G

SeqID:141/142 Hepcidin antimicrobial peptide

SeqID:239/240Metallothionein 1H

EGL Nine (C.elegans) Homolog 3

As described above, it has been discovered that a polypeptide encoded by a gene identified from the EST recited in SEQ ID No 86, having the Protein accession number BAB15101 (encoded by Homo sapiens cDNA: FLJ21620 fis, clone COL07838 Nucleotide accession AK025273) is regulated by hypoxia. Other public domain sequences corresponding to this gene include Homo sapiens cDNA: FLJ23265 fis, clone COL06456 Nucleotide accession AK026918. Accordingly, when referring in the present specification to the EST recited in SEQ ID No 86, it is intended that these gene and protein sequences are also embraced. This gene was identified using Research Genetics Human GeneFilters arrays, which contain an EST corresponding to the gene (accession number R00332). The gene is now termed EGL nine (C.elegans) homolog 3.

There are no reports that describe the function of this human gene. However, a high degree of amino acid homology is observed between the protein encoded by this gene, and a rat protein called “Growth factor responsive smooth muscle protein” or “SM20” (Nucleotide accession U06713; Protein accession A53770). An alignment of single letter amino acid sequences shown below. Over the italicised region there is 97% amino acid similarity and 96% amino acid identity.

| | | |
|---|---|---|
| A53770 | (1) | MTLRSRRGFLSFLPGLRPPRRWLRISKRGPPTSHWASPALGGRTLHYSCR |
| BAB15101 | (1) | -------------------------------------------------- |
| | | 51 100 |
| A53770 | (51) | SQSGTPFSSEFQATFPAFAAKVARGPWLPQVVEPPARLSASPLCVRSGQA |
| BAB15101 | (1) | |
| | | 101 150 |
| A53770 | (101) | LGACTLGVPRLGSVSEMPLGHIMRLDLEKIALEYIVPCLHEVGFCYLDNF |
| BAB15101 | (1) | ----------------MPLCHIMRLDLEKIALEYIVPCLHEVGFCYLDNF |
| | | 151 200 |
| A53770 | (151) | LGEVVGDCVLERVKQLHYNGALRDGQLAGPRAGVSKRHLRQDQITWIGGN |
| BAB15101 | (35) | LGEVVGDCVLERVKQLHCTGALRDGQLAGPRAGVSKRHLRGDQITWIGGN |
| | | 201 250 |
| A53770 | (201) | EEGCEAINFLLSLIDRLVLYCGSRLGKYYVKERSKAMVACYPGNGTGYVR |
| BAB15101 | (85) | EECCEAISFLLSLIDRLVLYCGSRLGKYYVKERSKAMVACYPCNCTGYVR |
| | | 251 300 |
| A53770 | (251) | HVDNPNGDGRCITCIYYLNKNWDAKLHGGVLRIFPEGKSFVADVEPIFDR |
| BAB15101 | (135) | HVDNPNGDGRCITCIYYLNKNWDAKLHGGILRIFPEGKSFIADVEPIFDR |
| | | 301 350 |
| A53770 | (301) | LLFFWSDRRNPHEVQPSYATRYAMTVWYFDAEERAEAKKKFRNLTRKTES |
| BAB15101 | (185) LLFFWSDRRNPHEVQPSYATRYAMTVWYFDAEERAEAKKKFRNLTRKTES | |
| | | 351 |
| A53770 | (351) | ALAKD |
| BAB15101 | (235) | ALTED |

The high degree of amino acid similarity suggests that the human protein BAB15101 has an equivalent biochemical function to the rat protein A53770 (“Growth factor responsive smooth muscle protein” or “SM20”). Recent publications have shown that SM20 functions to promote apoptosis in neurons (Lipscomb et al., J Neurochem 1999; 73(1):429-32; Lipscomb et al., J Biol Chem Nov. 1, 2000; [epub ahead of print]). Significantly, SM20 has been shown to be expressed at high levels in the heart (Wax et al., J Biol Chem 1994; 269(17): 13041-7).

It has also been discovered that a polypeptide encoded by a gene identified from the EST recited in SEQ ID No 90, having the Protein accession number CAB81622, is regulated by hypoxia. The encoding human gene has been annotated in the UniGene database as “Similar to rat smooth muscle protein SM-20”; the nucleotide sequence is contained within the nucleotide accession AL117352. More recently, a longer fragment of this gene has been cloned, named c1orf12, or EGLN1 (Nucleotide accession AAG34568; Protein accession AAG34568). Accordingly, when referring in the present specification to the EST recited in SEQ ID No 90, it is intended that these gene and protein sequences are also embraced.

This distinct human gene, encoding a protein related to SM20 and EGLN3 (BAB15101), is also induced in response to hypoxia. This gene was identified using Research Genetics Human GeneFilters arrays, which contain an EST corresponding to the gene (accession number H56028).

Independently to this, a fragment of this gene has been cloned from a cDNA library derived from hypoxic human cardiomyoblasts, and it has been shown that the gene is increased in expression in response to hypoxia in this cell type (see Table 1 herein; penultimate row). The nucleotide sequence of this cDNA fragment is referred to herein as SEQ ID No 90a.

In the light of this novel discovery reported herein that these human equivalents of SM20 are induced by hypoxia, it is herein proposed that in cardiac ischaemia, the resulting apoptosis is due at least in part, to increased expression of these genes. The therapeutic modulation of the activity of EGLN3 (BAB15101; SEQ ID NO: 85), the EGLN3 splice variant (SEQ ID NO: 85a), EGLN1 (c1orf12; AAG34568; SEQ ID NO: 89), CAB81622, SM20 and other equivalent proteins and encoding genes therefore provides a novel means for the treatment of myocardial ischaemia, through the alteration of the propensity of myocardial cells to undergo apoptosis. For example, a suitable treatment may involve altering the susceptibility of ischaemic myocardial tissue to subsequent reperfusion and re-oxygenation, or may involve modulating the susceptibility of chronic ischaemic myocardial tissue (including forms of angina) to later more severe ischaemia, which would result in myocardial infarction. It is submitted that, by way of analogy, cerebral ischaemia may be treated using the same principle.

Although the Applicant does not wish to be bound by this theory, the downstream effects of SM20 and related genes such as EGLN3 (BAB15101; SEQ ID NO: 85), the EGLN3 splice variant (SEQ ID NO: 85a), EGLN1 (c1orf12; AAG34568; SEQ ID NO: 89), CAB81622 and SM20, namely, apoptosis and angiogenesis might be explained as follows. The apoptotic effect of NGF withdrawal may be mediated by induction of the hypoxia pathway, but may be an aspect of the supposed involvement of the HIF protein in the stress response. HIF1α is induced by reactive oxygen species (see Richard et al. J Biol Chem Sep. 1, 2000;275(35):26765-71). This could, in turn, be mediated by over-load of the proteosomal pathway for HIF1α degradation and the consequent accumulation of undegraded HIF1α. Accordingly, it is considered that modulation of SM20 and the related genes EGLN3 (BAB15101; SEQ ID NO: 85), the EGLN3 splice variant (SEQ ID NO: 85a), EGLN1 (c1orf12; AAG34568; SEQ ID NO: 89) and CAB81622 may have applications in the treatment of diseases resulting from disturbances in proteosome function, such as prion diseases and other neuro-degenerative diseases.

These data provide the first connection between these related genes and the physiological response to hypoxia. Recently published research papers have identified that the protein products of these genes can act as proline hydroxylases (see Bruick R K et al Science. 2001 294:1337-40 and Epstein A C et al Cell. 107:43-54). This is consistent with our observations that certain proline hydroxylases are induced in response to hypoxia and the genes EGLN1 and EGLN3 are part of the hypoxia response. For example, two genes encoding proline hydroxylases have been identified herein as being increased in expression in response to hypoxia (proline 4-hydroxylase, alpha polypeptide 1; SeqID:231/232, proline 4-hydroxylase, alpha polypeptide II; SeqID:349/350). This identified a functional significance of proline hydroxylation as a response to hypoxia.

Proline hydroxylase leads to degradation of HIF1α in normoxia (HIF regulates its own degradation—feedback). Hydroxylated HIF1α+VHL leads to ubquitination and consequent degradation of HIF1α by proteosome. The activity of the prolyl hydroxylase is 0₂-dependent, so under conditions of hypoxia, HIF1α is not hydroxylated efficiently and is stabilized. HIF1α protein thus accumulates to a high level. The hypoxia-induction of the prolyl hydroxylase ensures that when 0₂ concentration returns to normal, there is sufficient enzyme available to target this high level of HIF1α efficiently for rapid degradation.

Degradation of HIF1α is dependent on HIF1-induced transcription (i.e. is hypoxia inducible). Berra et al (FEBS Lett Feb. 23, 2001;491(1-2):85-90) raises the specific hypothesis of an unknown hypoxia-inducible factor which targets HIF1a for proteosomal degradation. It appears reasonable to propose that this factor will clearly be hypoxia-inducible, to ensure that a rapid and effective constraint on the hypoxic response would operate on return to normoxia. It now appears as if the genes EGLN1, EGLN3 and the EGLN3 splice variant form part of this mechanism.

It is also hypothesised that SM20 and the related genes EGLN3 (BAB15101; SEQ ID NO: 85), the EGLN3 splice variant (SEQ ID NO: 85a), EGLN1 (c1orf12; AAG34568; SEQ ID NO: 89) and CAB81622 may act as tetramers. Known prolyl hydroxylases such as prolyl 4-hydroxylase (P4H) are known to act as tetramers of two alpha subunits and two beta subunits. SM20 and the related genes exhibits high similarity to the alpha subunit of P4H and it therefore seems likely that SM20 and the related genes are likely to have a binding partner that is equivalent to the beta subunit of P4H.

SM20 has been shown to bind to the transcription factor HIF1α, and shares a low level homology with a p53 binding protein. P53 is a transcription factor that is known to be involved in apoptosis. Accordingly, it is proposed that in addition to binding to HIF1A, SM20 and the related genes EGLN3 (BAB115101; SEQ ID NO: 85), the EGLN3 splice variant (SEQ ID NO: 85a), EGLN1 (c1orf12; AAG34568; SEQ ID NO: 89) and CAB81622 may also bind and modify other transcription factors that are involved in the hypoxic response such as EPAS and HIF3A, or other transcription factors such as p53 and thereby influencing apoptosis. This aspect of the invention thus provides dimer and tetrameric forms of the EGLN3 (BAB15101; SEQ ID NO: 85), the EGLN3 splice variant (SEQ ID NO: 85a), EGLN1 (c1orf12; AAG34568; SEQ ID NO: 89) and CAB81622 proteins, preferably complexed with a protein selected from the group consisting of HIF1α, p53 and a protein binding partner that is equivalent to the beta subunit of P4H. Preferably, such dimers and tetramers are heterodimers/heterotetramers.

To provide further evidence that these related genes are a significant part of the hypoxia response additional expression data is presented here.

Expression profiles for these two genes will be displayed with pre-chip normalisation to correct for differences in RNA labelling etc, but within each gene no further normalisation is done (per-gene normalisation), so the relative absolute expression levels of the two genes can be compared and Y-axis units between separate graphs from the same experiment are comparable. These graphs are presented as FIGS. 32b (c1orf12) and 32c (EGLN3).

It can be seen from these Figures that both genes (c1orf12 and EGLN3) are inducible in response to hypoxia in macrophages whether activated by gamma interferon and lipopolysaccharide or if de-activated by treatment with interleukin-10. In macrophages the absolute expression level of C1orf12 appears to be higher than EGLN3.

There is a prejudice in the art that the response to hypoxia is generic to all cell types. Contrary to this, we show herein that genes are regulated by hypoxia to a greater degree in certain cell types, substantiating their utility in designing specific therapeutic products for diseases involving those cell types.

From FIGS. 32a and 32d and the data presented below, differing expression profiles of the two related genes c1ORF12 and EGLN3 are apparent throughout the 11 tested cell types, though C1orf12 is generally expressed at higher levels than EGLN3.

| | | mRNA | mRNA |
|---|---|---|---|
| | | expression | expression |
| | | (c1ORF12 | (EGLN3 |
| Cell type | Oxygen | SeqID: 89/90) | SeqID: 85/86) |
| adipocyte | normoxia | 0.0075 | 0.0033 |
| adipocyte | hypoxia 6 hr | 0.0091 | 0.0027 |
| adipocyte | hypoxia 18 hr | 0.0182 | 0.0025 |
| cardiomyocyte | normoxia | 0.0067 | 0.0019 |
| cardiomyocyte | hypoxia 6 hr | 0.0381 | 0.0023 |
| cardiomyocyte | hypoxia 18 hr | 0.0201 | 0.0026 |
| endothelial | normoxia | 0.0198 | 0.0019 |
| endothelial | hypoxia 6 hr | 0.0583 | 0.0033 |
| endothelial | hypoxia 18 hr | 0.0397 | 0.0026 |
| fibroblast | normoxia | 0.0119 | 0.0032 |
| fibroblast | hypoxia 6 hr | 0.0260 | 0.0046 |
| fibroblast | hypoxia 18 hr | 0.0235 | 0.0040 |
| hepatocyte | normoxia | 0.0075 | 0.0080 |
| hepatocyte | hypoxia 6 hr | 0.0074 | 0.0146 |
| macrophage | normoxia | 0.0033 | 0.0008 |
| macrophage | hypoxia 6 hr | 0.0083 | 0.0018 |
| macrophage | hypoxia 18 hr | 0.0058 | 0.0021 |
| mammary epithelial | normoxia | 0.0065 | 0.0014 |
| mammary epithelial | hypoxia 6 hr | 0.0137 | 0.0055 |
| mammary epithelial | hypoxia 18 hr | 0.0144 | 0.0065 |
| monocyte | normoxia | 0.0027 | 0.0006 |
| monocyte | hypoxia 6 hr | 0.0084 | 0.0014 |
| monocyte | hypoxia 18 hr | 0.0080 | 0.0016 |
| neuroblastoma | normoxia | 0.0344 | 0.0011 |
| neuroblastoma | hypoxia 6 hr | 0.1085 | 0.0013 |
| neuroblastoma | hypoxia 18 hr | 0.0551 | 0.0020 |
| renal epithelial | normoxia | 0.0275 | 0.0046 |
| renal epithelial | hypoxia 6 hr | 0.0560 | 0.0046 |
| renal epithelial | hypoxia 18 hr | 0.0395 | 0.0096 |
| skeletal myocyte | normoxia | 0.0088 | 0.0029 |
| skeletal myocyte | hypoxia 6 hr | 0.0277 | 0.0035 |
| skeletal myocyte | hypoxia 18 hr | 0.0245 | 0.0038 |

For instance, in the hypoxic hepatocyte (6 hr) the normalized expression values of EGLN and c1orf12 are 0.015 and 0.0074 respectively, i.e. EGLN being the dominant gene. In contrast, in the neuroblastoma cell line SH-SY5Y, the normalized expression values of EGLN and c1orf12 after 6 hr hypoxia are 0.0012 and 0.108 respectively, i.e. c1orf12 being the dominant gene by a large margin. This data demonstrates that c1ORF12 and EGLN3 are not constitutively expressed at an equal amount in different tissues indicating specificity of function. Therefore, it is considered that therapeutic products may be developed based on this data, with the goal of modulating proline hydroxylation of target proteins (such as HIF1alpha) in specific tissues, based on the differing expression profile of c1ORF12 and EGLN3 in those tissues.

In Example 1b herein, genes were identified from a custom array, which give a greater induction in macrophages (by a factor of at least 1.5) when hypoxia is augmented by over-expression of HIF1alpha or EPAS from an adenovirus. The data from the HIF/EPAS over-expression work is presented herein in Example 1c, but specifically relating to c1ORF12 and EGLN3 is summarised in FIGS. 32e and 32f. From this data it is apparent that EGLN3/FLJ21620 fis cl.COL07838 but not c1ORF12 is increased in expression by the transcription factor EPAS1, but not HIF1alpha. This is apparent by comparing experimental condition 9 (hypoxia with EPAS overexpression; expression value=3.48) to that of 5 (hypoxia without EPAS overexpression; expression value=1.65). This adds valuable information about the mechanism of regulation of the gene encoding EGLN3.

To confirm this data the RNA samples for experimental conditions 1,3,5,7,9 (corresponding to the high dose of adenovirus) were also measured using a different array-based methodology—the AffyMetrix GeneChip. The results of this experiment are presented in FIGS. 32g and 32h.

Functional Characterization of EGL nine (C.elegans) homolog 3 role in the induction of Cardiomyocyte apoptotic cell death

EGLN3 has been cloned into pONY8.1 and Smart2.IRES.GFP equine infectious anaemia virus (EIAV) vectors, and AdCMV.TRACK.GFP (AdenoQuest) adenoviral genome vectors (see co-owned co-pending International patent application PCT/GB01/00758). These vectors have been used in “gain-of-function” studies in which EGLN3 has been overexpressed in order to elucidate corresponding protein function. Human embryo kidney (HEK 293T) and dog osteosarcoma (D17) cell lines have been used in transient plasmid transfection experiments to confirm EGLN3 expression from viral vector genomes. Rat cardiomyocyte cell line (H9C2) and primary human neonatal cardiomyocytes (PHNC) (BioWhittaker, CC2582) have been used in viral transduction experiments to determine the biological activity of EGLN3. In all cell types, expression of EGLN3 has been followed by combinations of immunofluorescence, Western blotting and TaqMan quantitative PCR. Immunofluorescence and Western blotting employ an antibody specific for the FLAG epitope engineered into the 3′ terminus of EGL nine (C.elegans) homolog 3 (Sigmna, F3165). TaqMan quantitative PCR utilizes the SYBR Green method (Applied Biosystems).

Western blotting has confirmed the transient expression of EGLN3 from an EIAV genome construct in HEK 293T (expected size approx 717 bp, 26 Kda). Immunofluorescence has localized transient expression of EGL nine (C.elegans) homolog 3 from EIAV expression construct in HEK293T to the cytoplasm. Expression of EGL nine (C.elegans) homolog 3 is elevated after 4 hours exposure to hypoxic conditions (0.1% (v/v) oxygen), when compared to expression observed under normoxia (20% (v/v) oxygen) (see FIG. 32i). TaqMan primers have been designed and optimised for the initial measurement of EGL nine (C.elegans) homolog 3 expression in EIAV or Adenovirus transduced H9C2 and PHNC (Forward: TCATCGACAGGCTGGTCCTC; Reverse: GTTCCATTTCCCGGATAGAA). All findings at the RNA level are corroborated by immunofluorescence and Western blotting analyses at the protein level.

EIAV transduction of H9C2 and PHNC has been optimised with constructs containing green fluorescence protein (GFP) and LacZ reporter genes, using the VSVg envelope and a range of MOI between 10 and 100. GFP results were scored by fluorescence microscopy, while LacZ transductants were identified through the assay of β-galactosidase activity. An MOI of 50 transduced approximately 50% of the cell population.

EGLN3 is predicted to have pro-apoptotic activity in cardiomyocytes. Early, Mid and late phase apoptosis are characterized by translocation of membrane phospholipid phosphatidylserine (PS) from the inner face of the plasma membrane to the cell surface, activation of specific proteases (caspases) and fragmentation of DNA, respectively (Martin, S. J., et al., J. Exp. Med. 1995, 182, 1545-1556; Alnemri, E. S., et al., J. Cell. Biochem. 1997, 64, 33-42; Wylie, A. H., et al., Int. Rev. Cytol. 1980, 68, 251-306). Translocation of PS has been identified through use of ApoAlert kit (Clontech; K2025-1), which employs FITC-labelled antibodies to detect surface expression of the PS, Annexin V. Caspase activity has been followed using the homogeneous fluorimetric caspase assay (Roche; 3005372) which allows the quantification of caspase activity through the cleavage of a fluorescent substrate. DNA fragmentation has been estimated using the nuclear stain Hoescht 33345 (Sigma, B2261; and fluorescence microscopy to locate areas of chromatin condensation. Total viability of cell population has been quantified through measurement of the ability of mitochondrial reductase to metabolize the fluorescent substrate MTT (Sigma, M2128)(Levitz S. M & Diamond, R. D. J. Infect. Dis. November 1985; 152(5):938-45).

Conditions for early, mid and late stage apoptosis in H9C2 and PHNC have been defined using hypoxia and nutrient-depleted growth medium to mimic those ischaemic conditions found in vivo (Brar, B. K., et al., J. Biol. Chem. 2000, 275, 8508-8514). Transduction of PHNC with EIAV vectors containing EGLN3 is sufficient to cause an increase in caspase activity in cells cultured under normoxic conditions, confirming the role of EGLN3 in the induction of cardiomyocyte apoptosis. Using an MOI of 50, a 2-fold increase in caspase activity was seen in EGLN3 transduced cells, when compared to controls 48 hours,post transduction (see FIG. 32j).

Increased expression of EGL nine (C.elegans) homolog 3 in transduced cells is confirmed by TaqMan, immunofluorescence and Western blotting. Similar experiments are performed to determine whether EGL nine (C.elegans) homolog 3 expression further sensitises H9C2 and PHNC to previously defined ischaemic insults. Staurosporine (Calbiochem; 569397) and Smart2.IRES.GFP EIAV vectors containing the Bax gene will be applied as chemical and viral pro-apoptotic controls, respectively (Yue, T-L., et al., J. Mol. Cell. Cardiol. 1998, 30, 495-507; Reed, J. C. J Cell Biol. 1994, 124(1-2):1-6).

Gene silencing approaches may be undertaken to down-regulate endogenous expression of EGLN3 in PHNC to determine the degree of protection against apoptotic cell death provided by a reduction in EGLN3 activity. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression. A Hammerhead ribozyme library, contained in EIAV expression vectors, may also be applied. Efficacy of both gene silencing approaches may be assessed initially through the measurement of EGLN3 expression, at the RNA level by TaqMan and at the protein level by Western blotting. Protection against previously described ischaemic insults provided by these methods of EGLN3 gene silencing may be assayed biologically as detailed above. Caspase inhibitors (caspase 3 inhibitor V, 2129002 and caspase inhibitor I, 627610, both Calbiochem) and Smart2.IRES.GFP EIAV vectors containing the Bc1-2 gene may be applied as chemical and viral anti-apoptotic controls, respectively (Kroemer, G. Nat Med. 1997, 3(6):614-20).

Similar “gain-of-function” and gene silencing approaches will be applied to the related gene, encoded by SEQ ID 90, named c1orf12.

PCR results for the EGLN3 splice variant (SEQ ID NO: 85a)

Initial work using RNase protection assays using a 32P-labelled riboprobe which spans the sequence of both isoforms validated the existence of the unexpected isoform (data not shown).

Qualitative RT-PCR was then used to estimate the relative abundance of the two splice forms of EGLN3 in a range of primary human cell types growing in normoxia and hypoxia, as described below. This method was also used to clone and sequence the splice isoform to demonstrate qualitative comparisons in the levels of the two isoforms.

This data gives further proof that the splice isoform actually exists, but moreover demonstrates that the relative expression levels of the two isoforms varies between cell types. It is therefore clear that cell type differential alternative splicing occurs for this gene. This may allow the design of more specific inhibitors to the different splice isoforms, which may have significant implications for the treatment and diagnosis of human disease. There are many examples of the significance of splicing to human disease, where the products of different splice forms having distinct biochemical and biological activity.

Primary human cell types were obtained from Clonetics (distributed by BioWhittaker, Walkersville, Md.) and cultured according to the manufacturer's recommendations, unless where otherwise shown.

RT-PCR was performed by reverse transcribing 2 ug total RNA with Superscript II reverse transcriptase (Invitrogen) in a 20 ul reaction. 1 ul of the resulting cDNA was used as template for PCR reactions using Clontech Advantage II polymerase. Primer nucleotide sequences were as follows:

Sense ctcgattctgcgggcgagatgc

Antisense gtcttcagtgagggcagattcag

PCR cycling was performed using an Applied Biosystems 9700, using the following “touchdown” cycling parameters:

(94° 1 min)×1

(94° 10 sec; 72° 2 min)×5

(94° 10 sec; 70° 20 sec; 72° 2 min)×5

(94° 10 sec; 68° 20 sec; 72° 2 min)×22

It should be noted that the combination of an accurate and processive reverse transcriptase enzyme (Superscript II) and highly stringent touchdown PCR cycling parameters as shown above, and a high fidelity hot-started enzyme (Advantage II polymerase) render these data highly specific and not liable to artefacts, as experienced with PCR amplifications performed under less stringent conditions.

Using these conditions, the expected ELGN3 PCR product of 735 bp is observed, though an additional band of 453 bp is also seen, representing the novel splice isoform described herein. This band was cut out, cloned and sequenced. The band has also been found in a variety of screened human cDNA libraries.

Evaluation of Potential HIF Prolyl Hydroxylase Inhibitors

As discussed above, the stability of HIF is controlled by proline hydroxylation, catalysed by the product of the EGLN3 gene which renders the HIF protein unstable. EGLN3 is only active in normoxia since the reaction that it catalyses requires oxygen. By this mechanism, in normoxia HIF is degraded and its target genes are not expressed (Epstein A C et al, Cell. 2001; 107(l):43-54). Analysis of the sequence of the novel splice isoform of EGLN3, contained in this specification, suggests that it retains an intact catalytic domain, and therefore should be active as a HIF proline hydroxylase. Here we have experimentally verified this.

Although endogenous HIF is unstable in normoxia, when it is overexpressed by experimental means, it is able to overcome the degradation mechanism and transcription of HIF target genes occurs. The commonly used human cell line 293T growing in conditions of normoxia was transiently transfected with a reporter plasmid (HRE-Luc), which provides a measure of HIF activity in units of Luciferase activity. This plasmid is described in Boast K et al Hum Gene Ther. 1999; 10(13):2197-208.

Plasmids and conditions used were as follows:

| Inhibitor | Plasmid1 | Plasmid2 | Plasmid3 |
|---|---|---|---|
| — | pCIneo | pCIneo | CMV-Luc |
| — | pCIneo | pCIneo | HRE-Luc |
| DAU (2.5 μM) | pCIneo | pCIneo | HRE-Luc |
| MIMO | pCIneo | pCIneo | HRE-Luc |
| | pCIneo | CMV-SVFL1 | HRE-Luc |
| MIMO | pCIneo | CMV-SVFL1 | HRE-Luc |
| — | CMV-HIF | PCIneo | HRE-Luc |
| — | CMV-HIF | CMV-SVFL1 | HRE-Luc |
| MIMO | CMV-HIF | CMV-SVFL1 | HRE-Luc |
| — | pCIneo | CMV-SVFL2 | HRE-Luc |
| MIMO | pCIneo | CMV-SVFL2 | HRE-Luc |
| — | CMV-HIF | CMV-SVFL2 | HRE-Luc |
| MIMO | CMV-HIF | CMV-SVFL2 | HRE-Luc |

MIMO=L-mimosine; 0.4 mM used. DAU=Daunorubicin; due to toxicity noted in a previous experiment, dosage of Daunorubicin used was 2.5 μM. Plasmid CMV-SVFL1 expresses full length EGLN3. Plasmid CMV-SVFL2 expresses the EGLN3 splice variant.

Three plasmid transfections were performed and pCIneo was used as the “stuffing”/control plasmid to fill the vacancies.

Transfected 293 cells were incubated under normoxia till the end of the experiment.

The results of this experiment are shown in FIG. 10. The collagen prolyl hydroxylase (PH) inhibitors EDB and MIMO were found to work as HIF-PH inhibitors under conditions of normoxia, even when the full length EGLN3 protein or EGLN3 splice variant was over-expressed.

It can be seen that when a plasmid containing HIF1α expressed from the CMV promoter was co-transfected with HRE-Luc, HIF1α activity was detected as 20953 Luciferase units. Accordingly, overexpressing HIF1α increased the luciferase activity (compare H and HM in FIG. 10). In a similar transfection, where a third plasmid containing full length EGLN3 (CMV-SVFL1) was also introduced, the units of luciferase produced were reduced to 918 (see col. S1HH in FIG. 10). This reflects the fact that EGLN3 targets HIF1a, including over-expressed HIF1α, for destruction leading to a decreased reading for HIF1α activity (measured here in Luciferase units). When this experiment was performed with the novel splice isoform of EGLN3 (CMV-SVFL2), as described herein, HIF1α was similarly inhibited to an even greater degree, with a Luciferase reading of 773 (see col S2HH in FIG. 10).

Over-expression of the full length EGLN3 protein or EGLN3 splice variant thus suppressed the effect of overexpression of HIF1α on luciferase. Both the full length EGLN3 protein and EGLN3 splice variant thus have been proven to possess biological activity. Overexpression of both these isoforms reduce HIF-mediated gene expression through HRE reporters, thus demonstrating their role in the HIF signalling pathway. The suppression effect of the EGLN3 splice variant appeared to be stronger than that of the full length EGLN3 protein.

Genes with a Greater Response in Renal Epithelial Cells

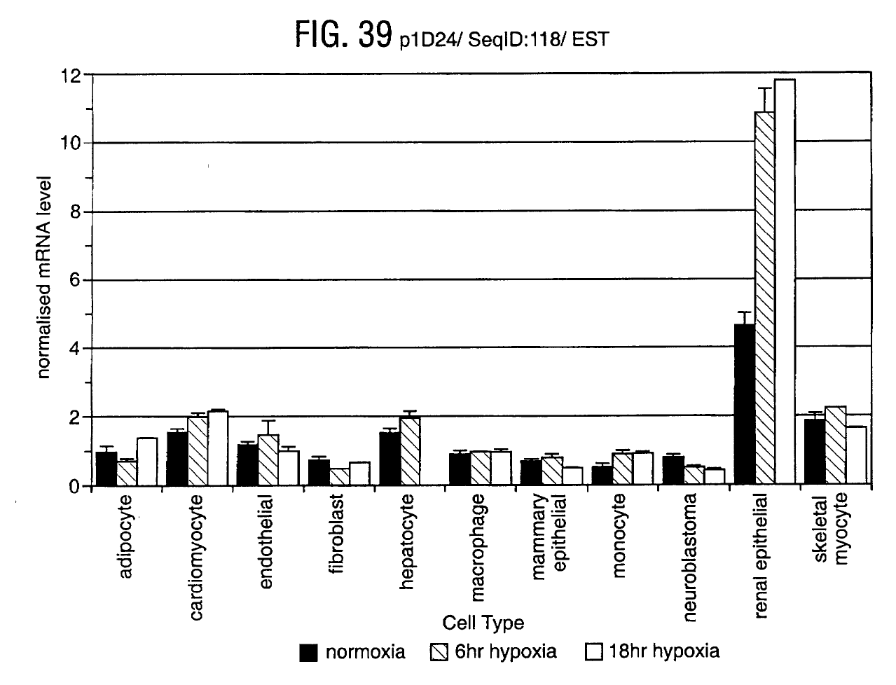

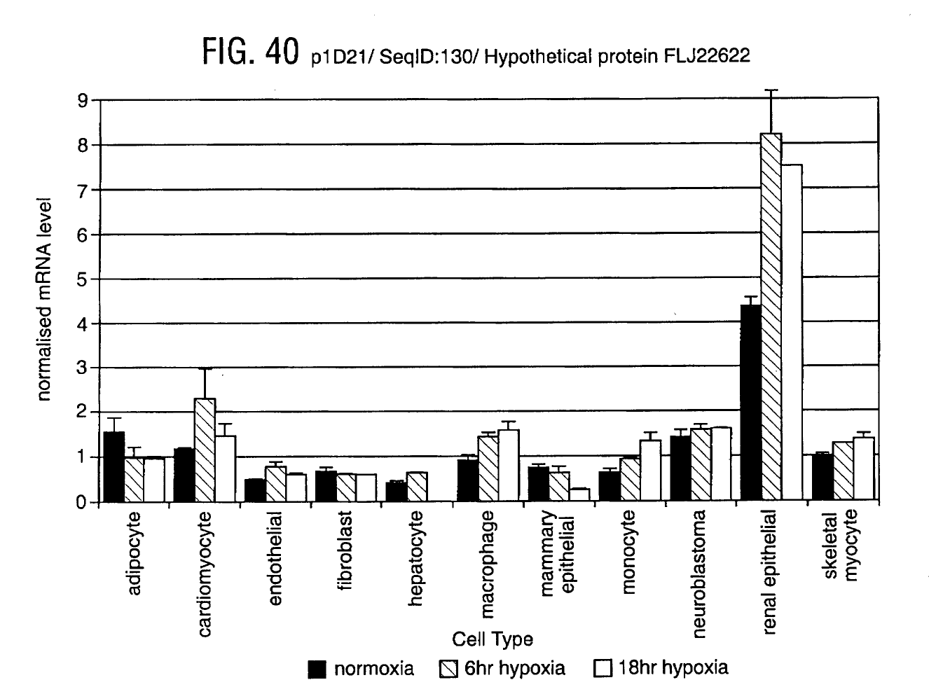

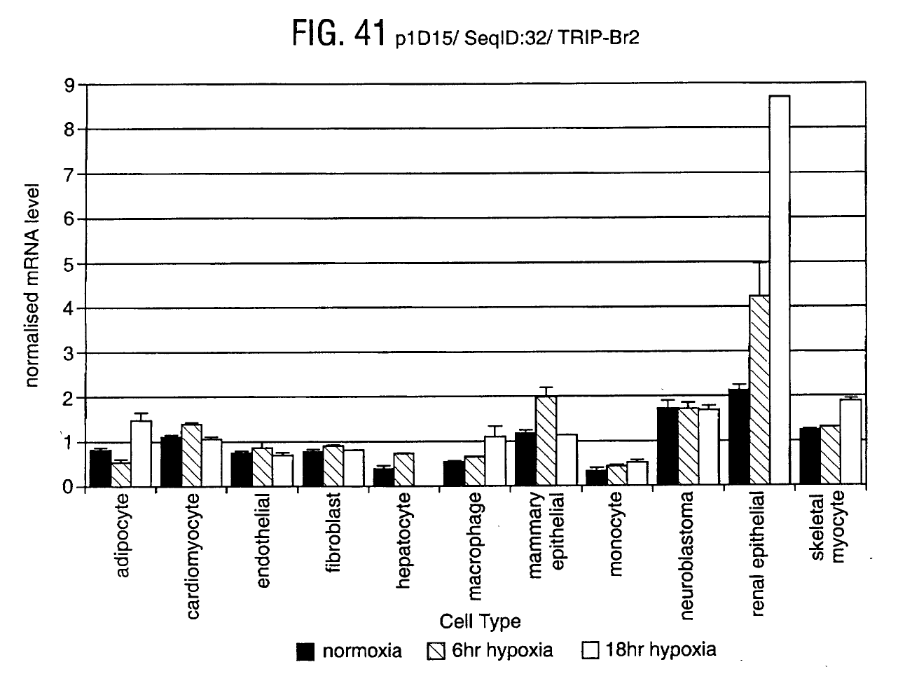

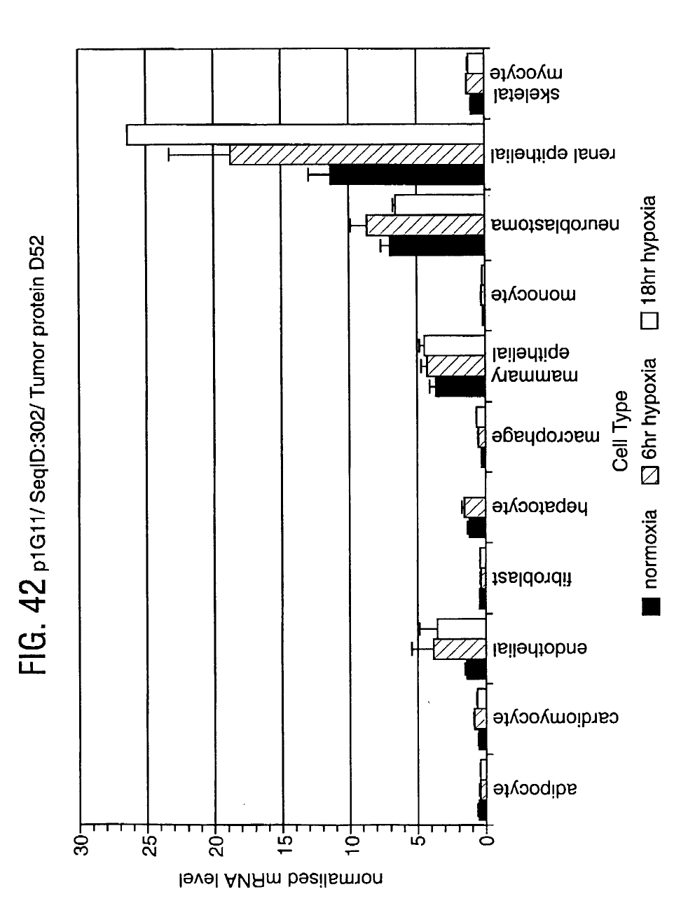

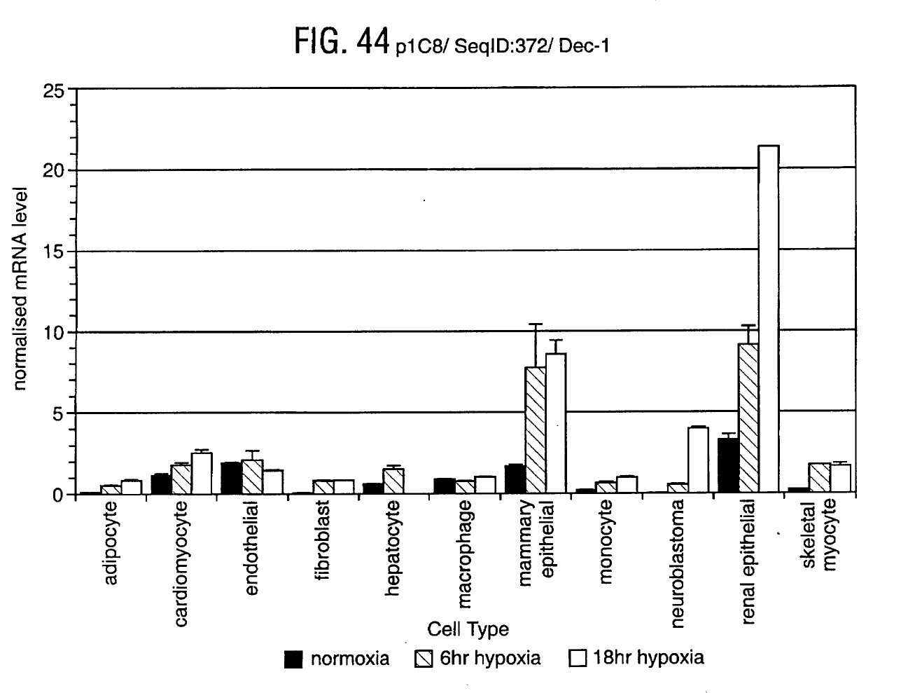

The dataset of Table 12 also contains genes which are induced preferentially in renal epithelial cells, in response to hypoxia. These genes are presented in FIGS. 39-44.

| | |
|---|---|
| SeqID: 117/118 | EST |
| SeqID: 129/130 | hypothetical protein FLJ22622 |
| SeqID: 31/32 | TRIP-Br2 |
| SeqID: 301/302 | Tumor protein D52 |
| SeqID: 91/92/92a | Semaphorin 4b |
| SeqID: 371/372 | Dec-1 |

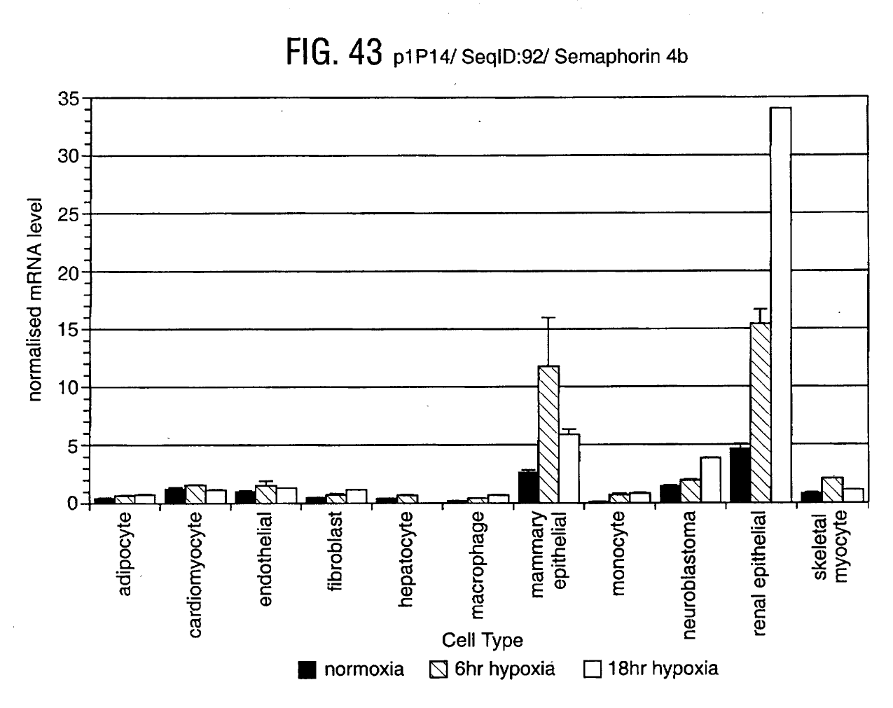

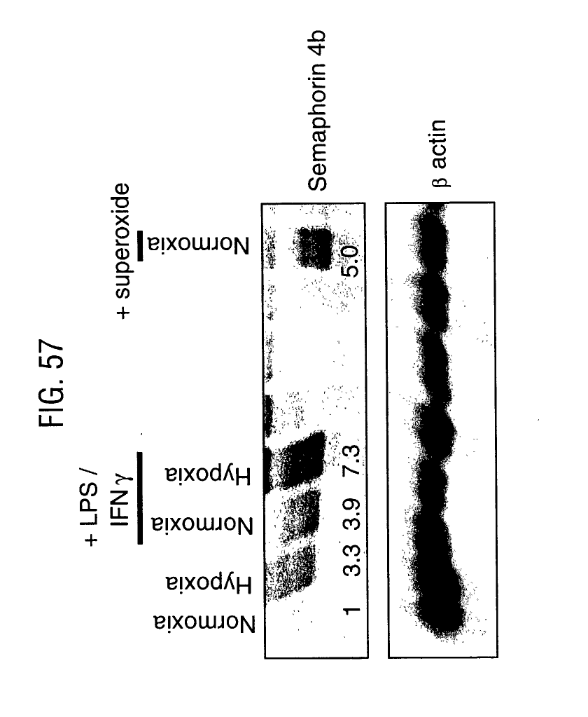

For Semaphorin 4b (SeqID:91/92/92a), the clone presented in FIG. 43 is p1P14, corresponding to IMAGE clone acc BE910319, the sequence of which covers a large region of the gene including protein coding sequence, which may cross-hybridise to other members of the semaphorin family. A separate clone (p1D17) as found in the original filing, was derived from the subtracted library and corresponds to a more unique region of this gene in the untranslated region. From Table 12 it will be appreciated that a significant response is also found in the macrophage. This is validated by RNase protection assay data (see FIG. 57). Further clarification of this gene using complementary experimentation methods will resolve the exact cell-type specific nature of the expression of this gene, though it is clear from this data that it is induced in renal epithelial cells and macrophages.

Genes with a Greater Response in Mammary Epithelial Cells

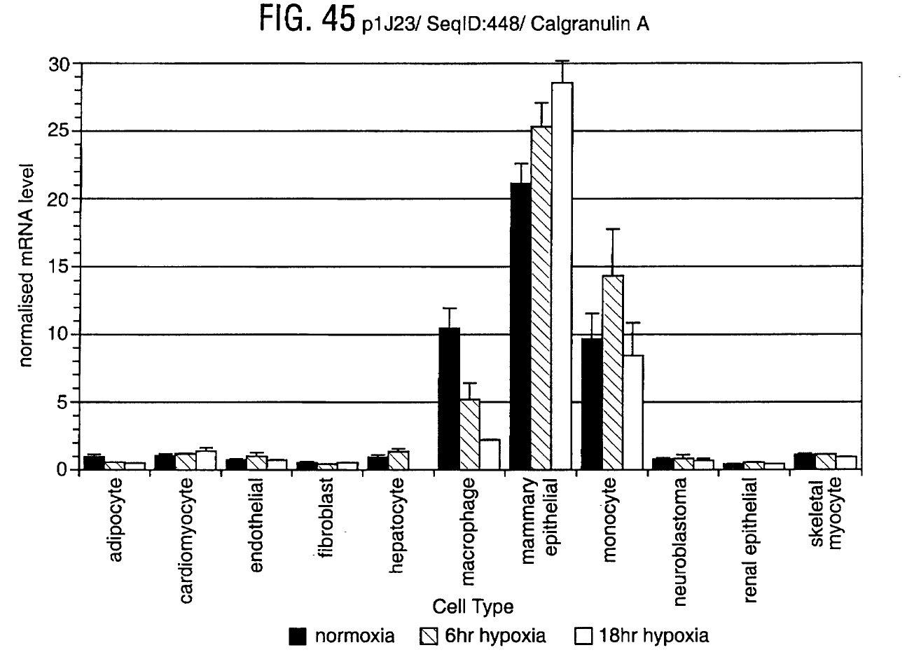

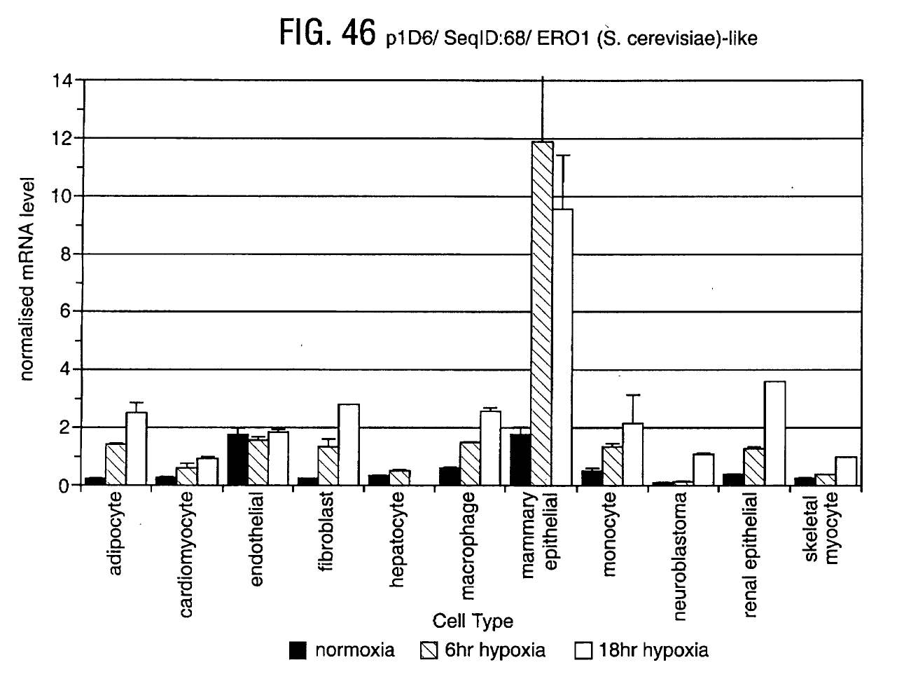

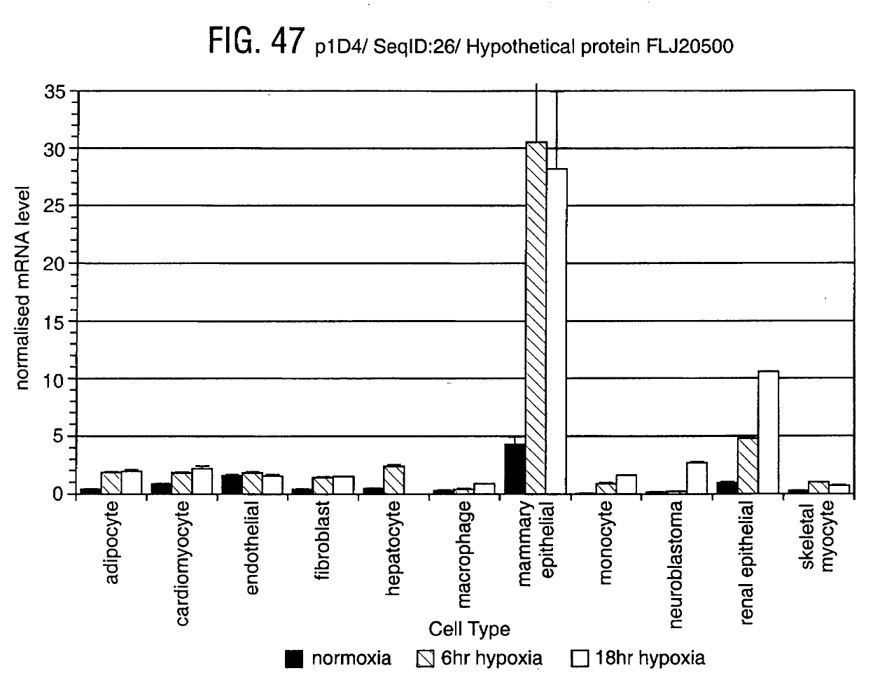

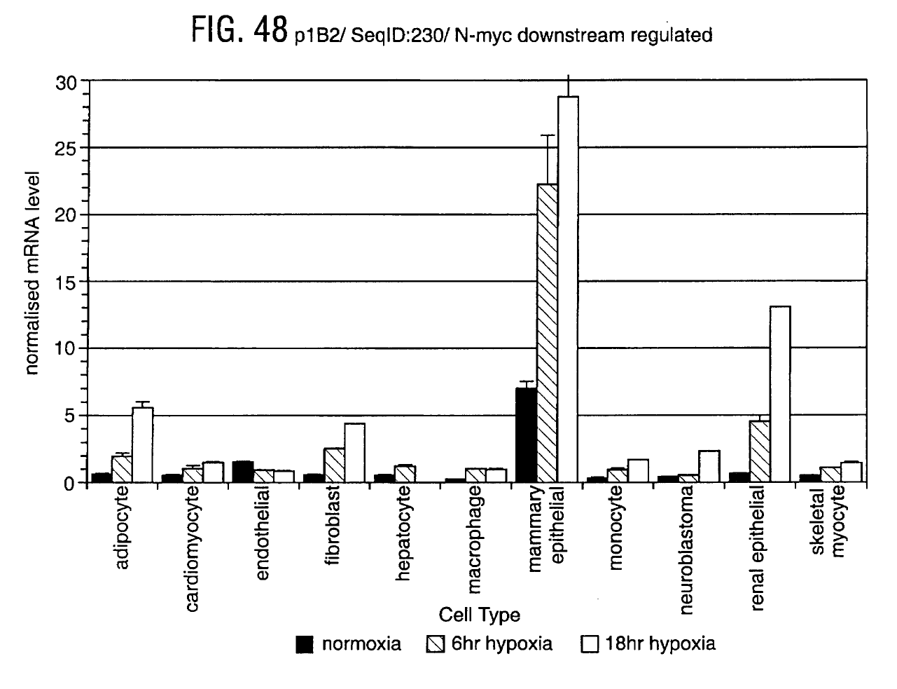

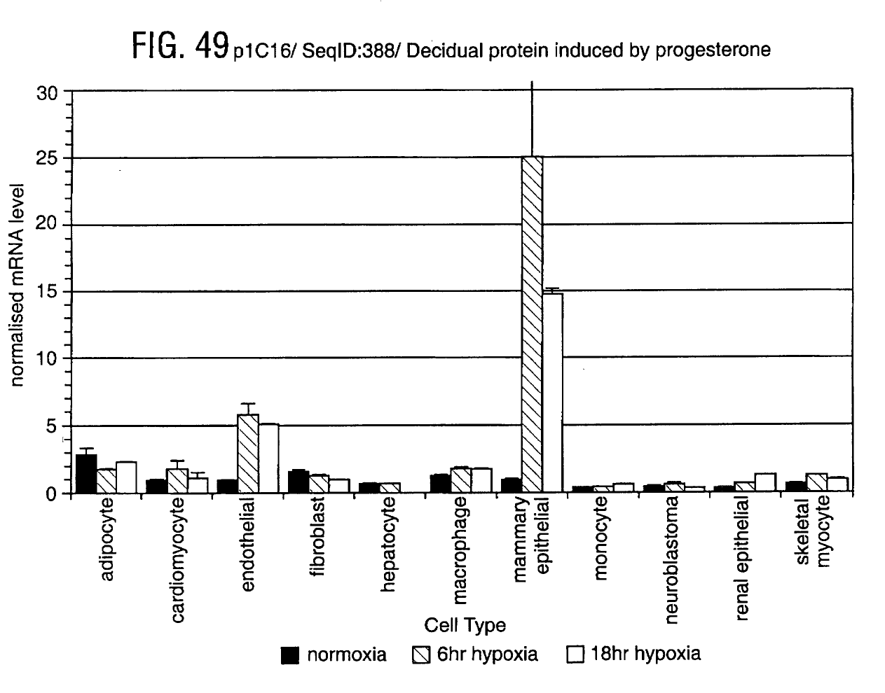

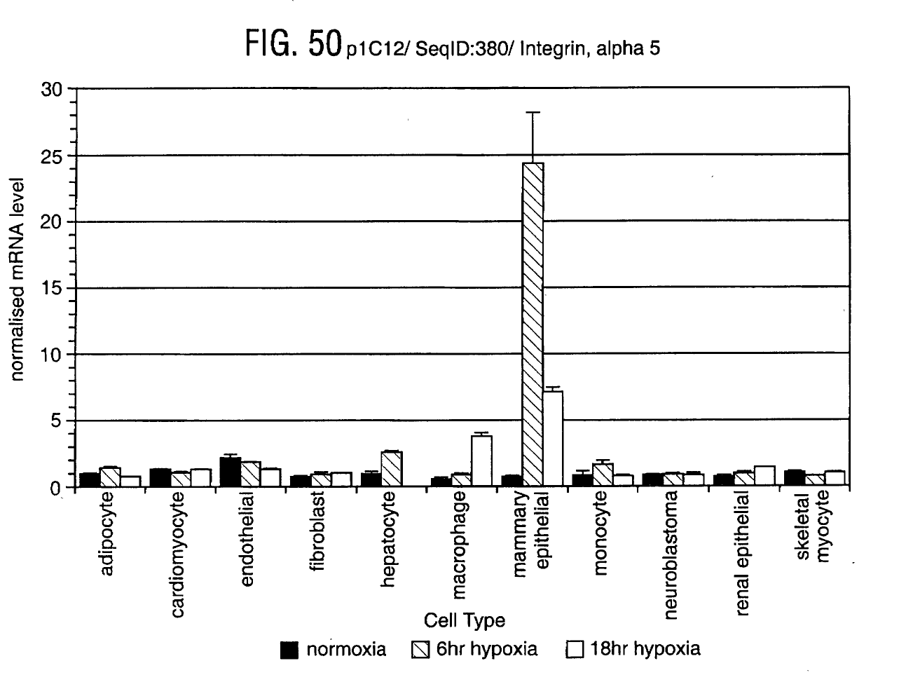

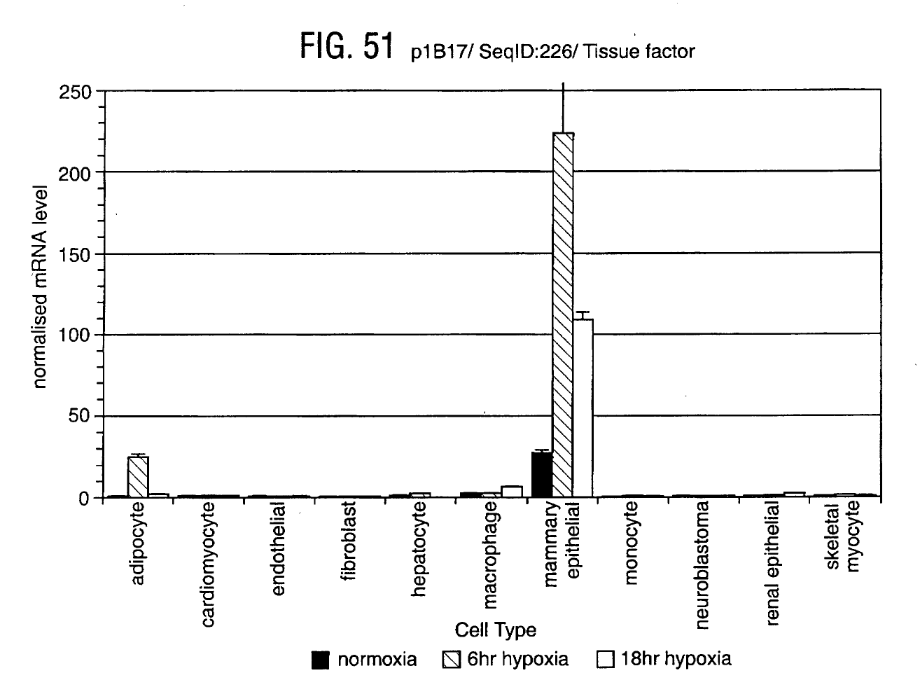

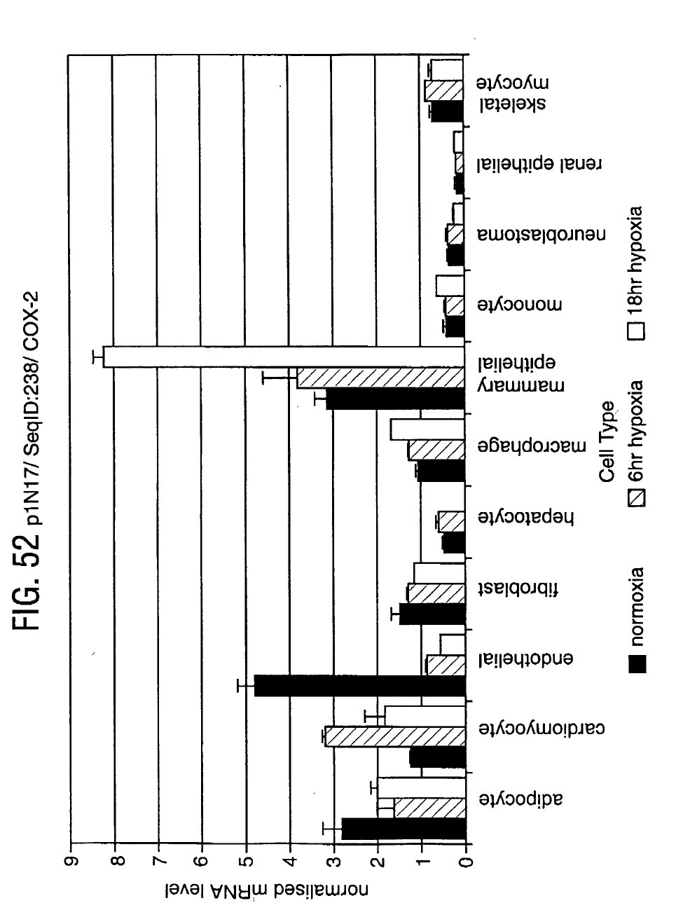

The dataset of Table 12 also contains genes which are induced preferentially in mammary epithelial cells, in response to hypoxia. These genes are presented in FIGS. 45-52.

| | |
|---|---|
| SeqID: 447/448 | Calgranulin A |
| SeqID: 67/68 | ERO1 (S. cerevisiae)-like |
| SeqID: 25/26 | hypothetical protein FLJ20500 |
| SeqID: 229/230 | N-myc downstream regulated |
| SeqID: 387/388 | Decidual protein induced by progesterone |
| SeqID: 379/380 | Integrin, alpha 5 |
| SeqID: 225/226 | Tissue factor |
| SeqID: 237/238 | COX-2 |

In the case of Cox-2, which encodes a key drug target, it can be seen that in many cell types, especially the mammary epithelial cells, there is a clear induction in response to hypoxia. In contrast, for endothelial cells there is a very significant time-dependent decrease in Cox-2 gene expression in response to hypoxia. Similarly, for Calgranulin A, there is strong positive induction in hypoxic mammary epithelial cells, but in the macrophage, the response to hypoxia is negative. These clearly exemplify the unexpected finding that cell types respond to hypoxia differentially, both quantitatively but also qualitatively. This is not currently known.

Hypoxia Regulation of Novel Human Genes

From Table 12, it will be appreciated that several genes with no prior annotation in public domain gene sequence databases are now identified as being regulated by hypoxia, in at least one cell type. To make this clear, these genes have been copied from Table 12 and presented in Tables 13 and 14), showing the hypoxia/normoxia induction ratio of the cell type in which the response is most pronounced. These figures are derived by dividing the normalized expression value, as found in Table 5, in hypoxia by that in normoxia for the same cell type. In some cases, where hypoxia causes inhibition of gene expression, the fold change is prefixed by the term “DOWN”. The cell type and time point of maximal response to hypoxia are also noted in Tables 13 and 14. The main purpose of Tables 13 and 14 is to demonstrate that these genes have significant responses to hypoxia per se.

In many cases, significant responses are seen in multiple cell types, though this data is not apparent here. In Table 13, the cDNA clones are currently un-annotated in public domain databases. In Table 14, the cDNA clones are currently annotated, but were not so as at the priority date.

### Example 4

Additional Disclosure of the Effect of Macrophage Activation on Hypoxia Regulation of Gene Expression

In Example 2, it is shown that activated and resting macrophages respond to hypoxia in different ways, showing that the hypoxia response is not a generic phenomenon. To consolidate this data, experiments were performed with the custom array, using additional experimental conditions and with a more in-depth analysis. Significantly, the expression values used are not simple hypoxia/normoxia ratios, done separately for macrophages of differing activation status, but rather the values used allow comparison of the relative expression levels throughout the entire set of experimental conditions. Hence, for any gene, all values throughout the entire set of experimental conditions are calculated by comparison to the median level of that gene throughout the dataset. This allows a clearer appreciation of the effects of hypoxia in the context of cell activation status. The following data demonstrates that of the newly discovered genes responsive to hypoxia, expression changes are also seen in response to key cytokines of the immune system, implying functions outside of the generic response to hypoxia and metabolism. This especially applies to unannotated genes, including ESTs and hypothetical proteins, showing potential functions in inflammation and angiogenesis on the basis of cytokine-regulation.

Macrophages were derived and cultured as described elsewhere in the specification. A total of 6 experimental conditions were analysed, as shown below. Where cells were treated with cytokines or hypoxia (0.1% oxygen), this was for 6 hr. Lipopolysaccharide (LPS) (from E.coli 026:B6; Sigma), gamma Interferon (IFN) and Interleukin-10 (IL-10) were all used at a final concentration of 100 ng/ml. The effect of gamma Interferon and Lipopolysaccharide is to activate macrophages, with a Th1 biased phenotype, as found in many inflammatory conditions. Interleukin-10 is a Th2 cytokine and de-activates macrophages, and suppresses their effector functions.

Experimental

| Condition | | |
|---|---|---|
| 1. | No cytokines | Normoxia |
| 2. | No cytokines | Hypoxia |
| 3. | IL-10 | Normoxia |
| 4. | IL-10 | Hypoxia |
| 5. | LPS + IFN | Normoxia |
| 6. | LPS + IFN | Hypoxia |

In Table 15, genes are shown which respond to LPS+IFN in normoxia by producing at least a 2-fold increase in expression, indicating probable pro-inflammatory functions. From this dataset various patterns of hypoxia regulation will be appreciated on top of the effect of LPS+IFN.

For instance, the gene SCYA8 (p1I21; SeqID:479/480) is decreased in expression by hypoxia, changing from 0.54 to 0.18 between conditions #1 and #2. In condition #5 (LPS+IFN normoxia), expression is dramatically increased to a value of 19.6. When LPS+IFN is combined with hypoxia, this increase is dampened-down to a value of 12.2. So for this example, hypoxia and cell activation have opposing effects on gene expression. A similar expression profile is found for several other genes in Table 15.

In contrast, the gene P8 protein-candidate of metastasis 1 (p1F17; SeqID: 329/330) is increased in expression by hypoxia, changing from 0.26 to 1.78 between conditions #1 and #2. In condition #5 (LPS+IFN normoxia) expression is increased from condition #1 to a value of 1.16. In condition #6, (LPS+IFN normoxia) the expression is further increased to a value of 2.59. So for this example, hypoxia and cell activation have similar effects on expression (i.e. increases) and these are found to be synergistic. A similar expression profile is found for several other genes in Table 15, including for Semaphorin 4b (p1P14; SeqID:91/92/92a), which has been independently verified by RNase protection assay (see FIG. 57).

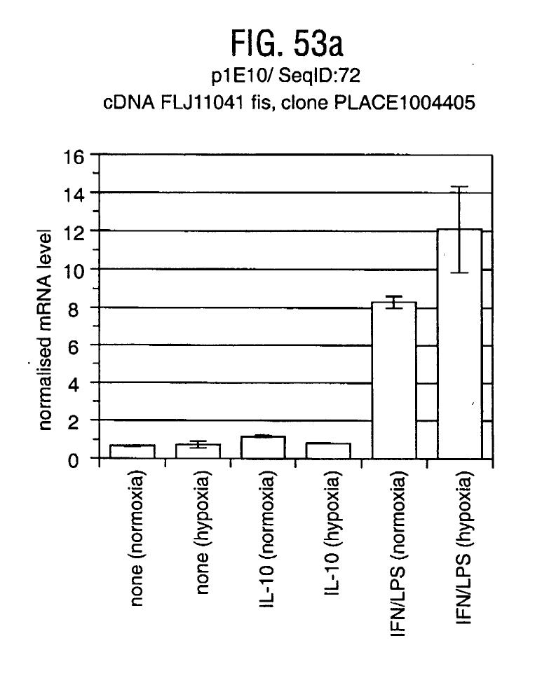

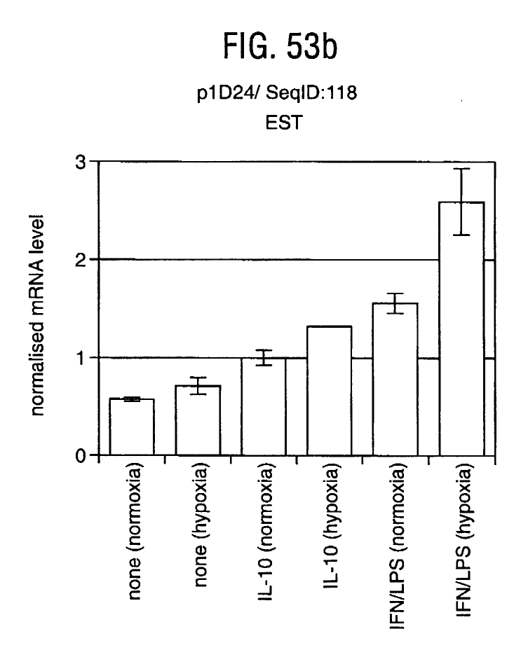

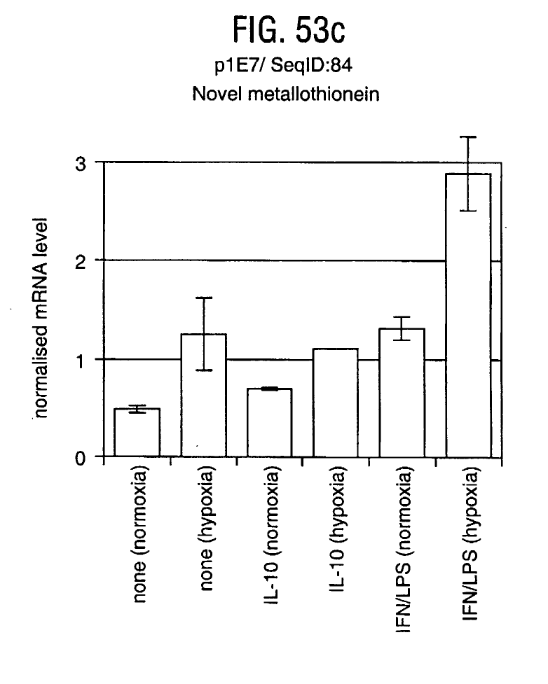

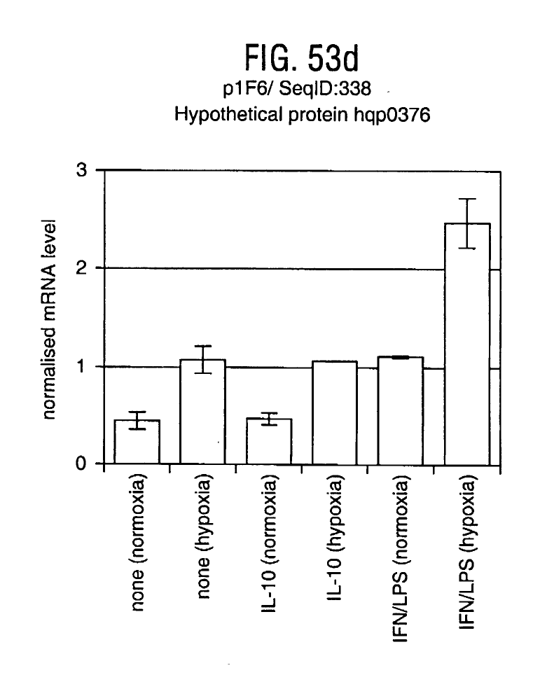

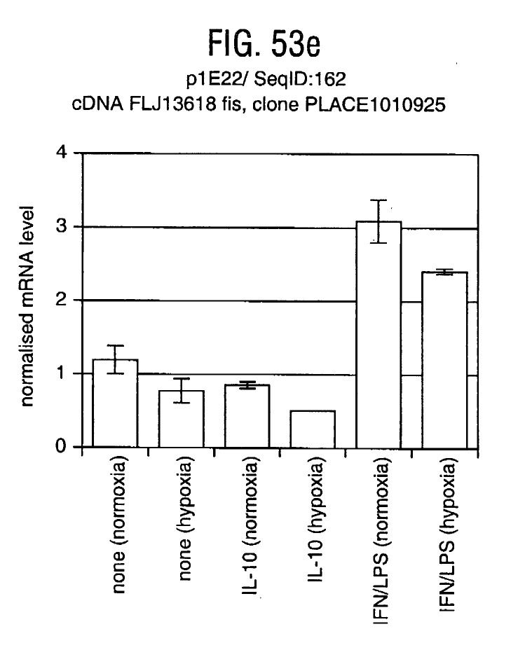

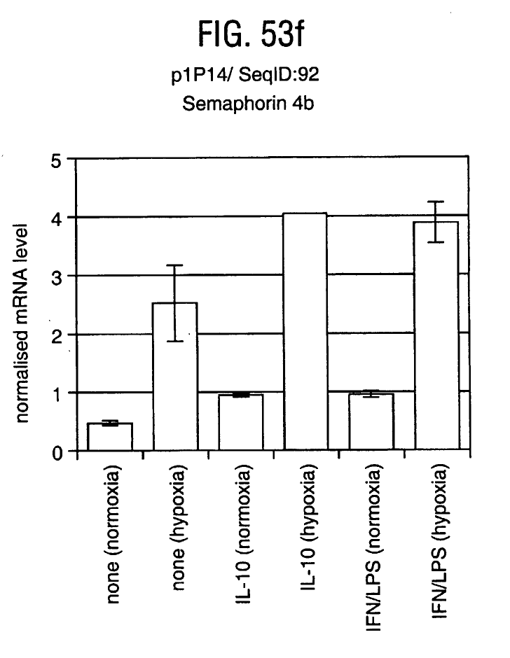

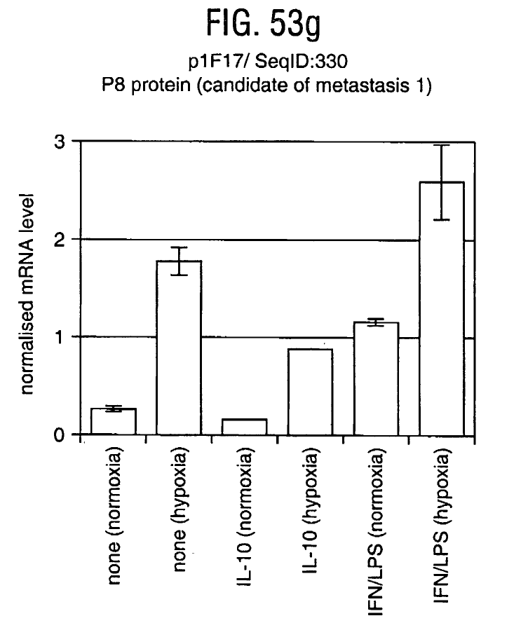

A selection of novel genes taken from Table 15 is also presented as FIG. 53. These novel genes are hence annotated here for the first time as being regulated not only by hypoxia, but also by Th1 inflammatory signals, as provided by LPS+IFN.

It will be appreciated that certain IMAGE clones were classed as novel and unannotated when the original patent filing was made (Dec. 8, 2000), but which can now be assigned to named genes. These are Uridine 5′ monophosphate hydrolase 1 (clone p1I7; SeqID:49/50) and Insulin induced protein 2 (clone p1D10; SeqID:75/76).

In Table 16, genes are shown which respond to LPS+IFN in normoxia by producing at least a 2-fold decrease in expression. From this dataset, various patterns of hypoxia regulation will be appreciated on top of the effect of LPS+IFN.

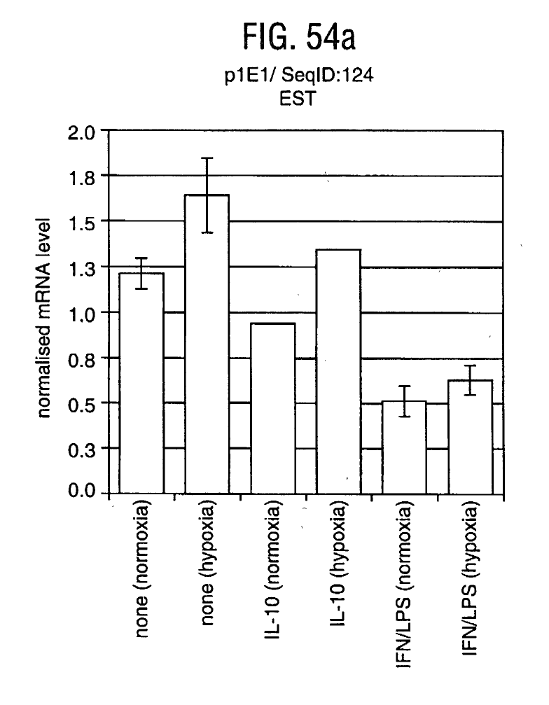

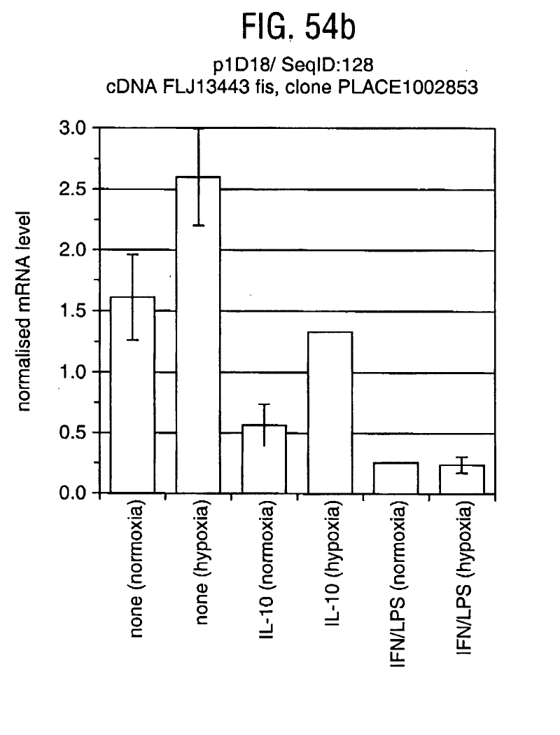

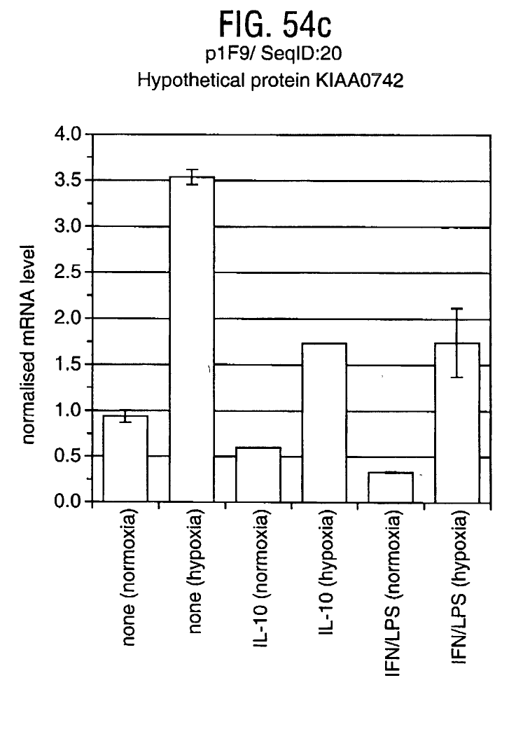

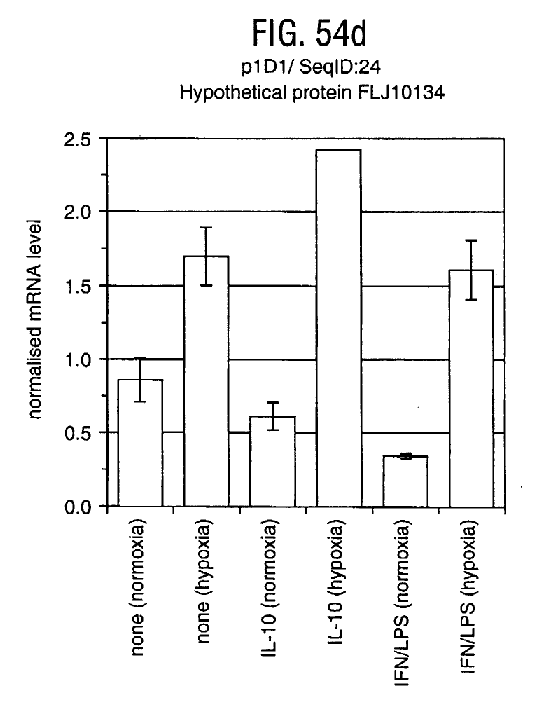

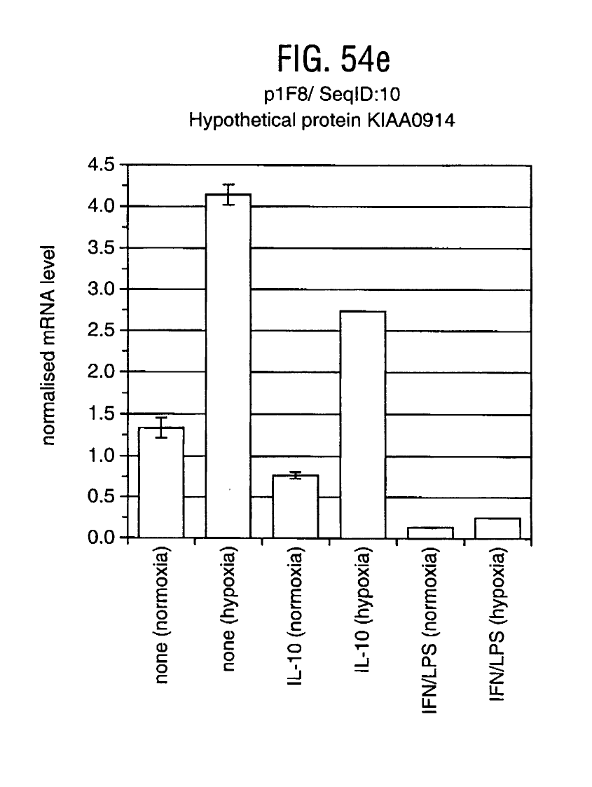

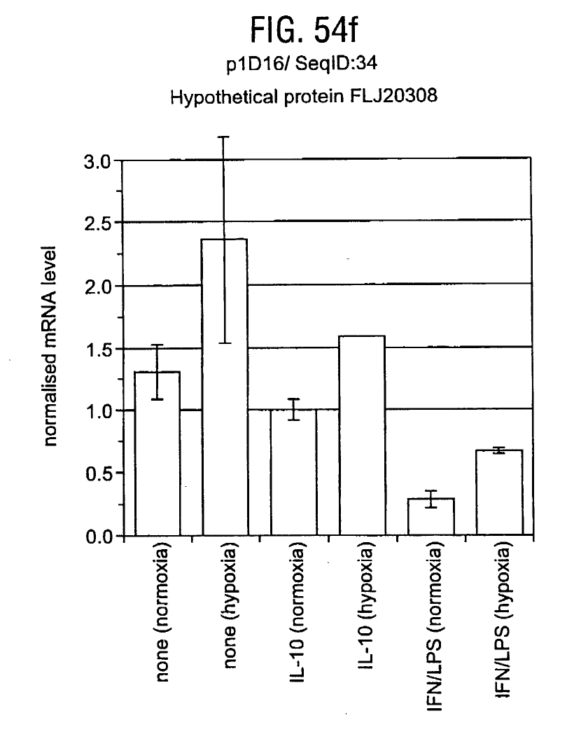

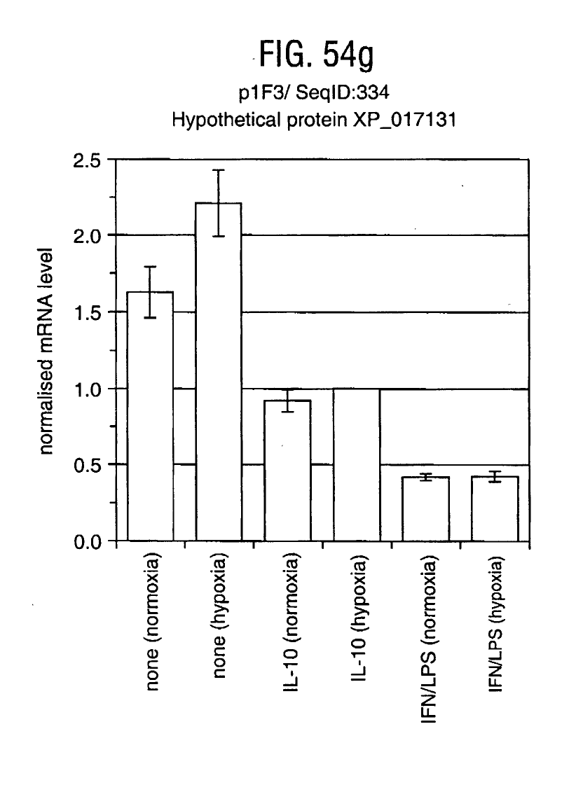

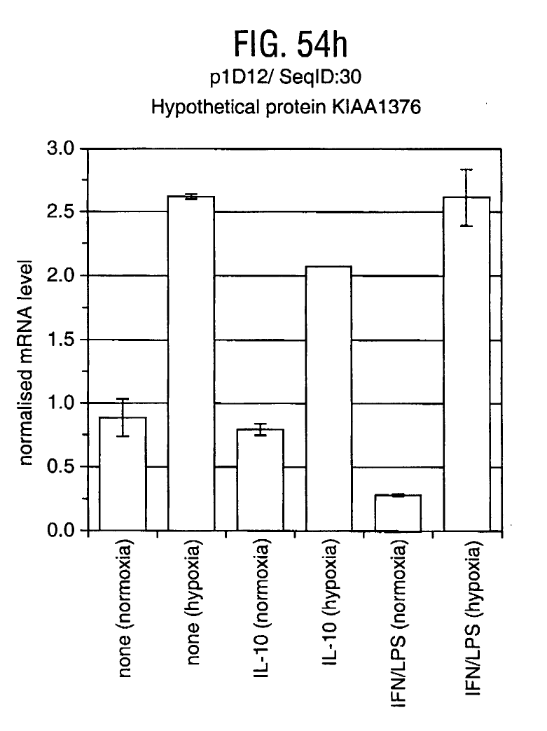

In FIG. 54, novel genes from Table 16 which are down-regulated by LPS+IFN and up-regulated by hypoxia are presented. For most of these, the combined effect of LPS+IFN AND hypoxia produces only a minor induction above the level of expression for activated normoxic cells (for example p1F8/SeqID:10/hypothetical Protein KIAA0914). In other cases, this is not the case, and hypoxia is able to over-ride the inhibitory effect of LPS+IFN on gene expression (for example p1D12/SeqID:30/hypothetical Protein KIAA1376). This clearly demonstrates the finding that different cell types or physiological states of a cell type (as here), respond to hypoxia differently.

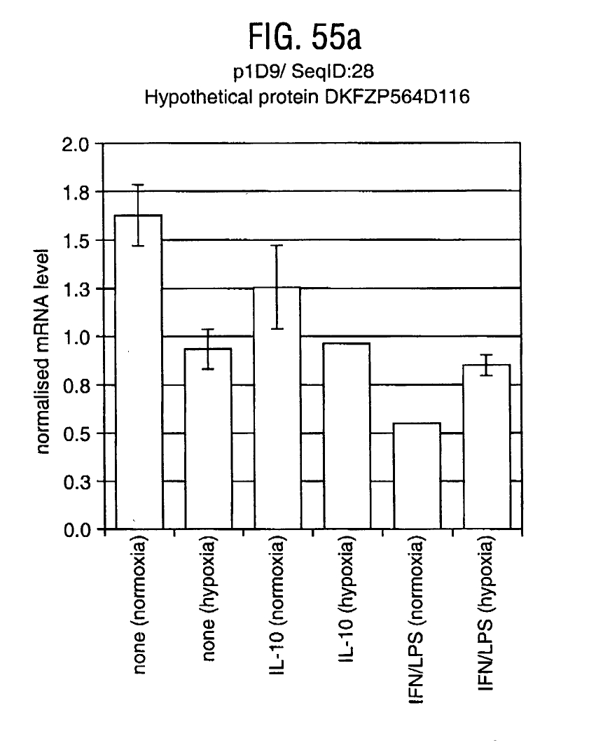

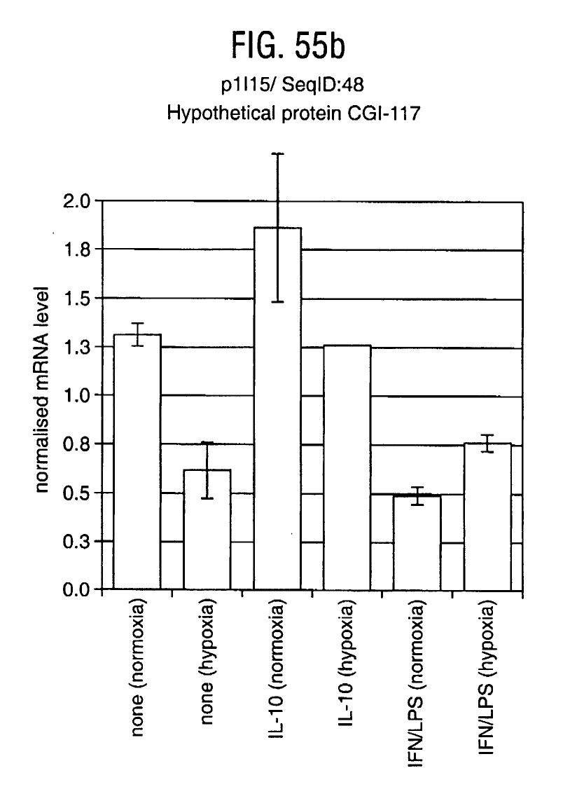

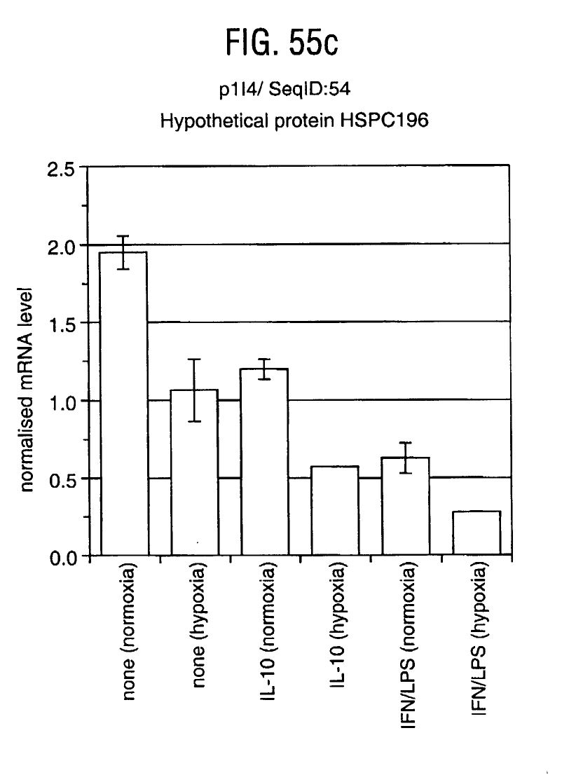

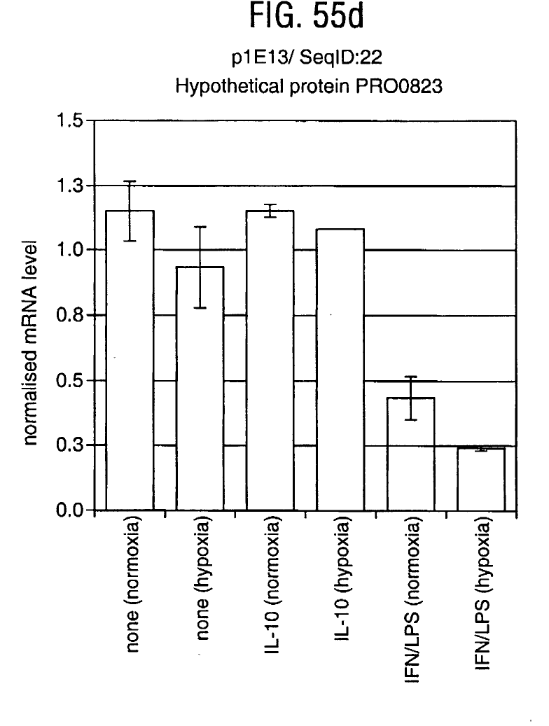

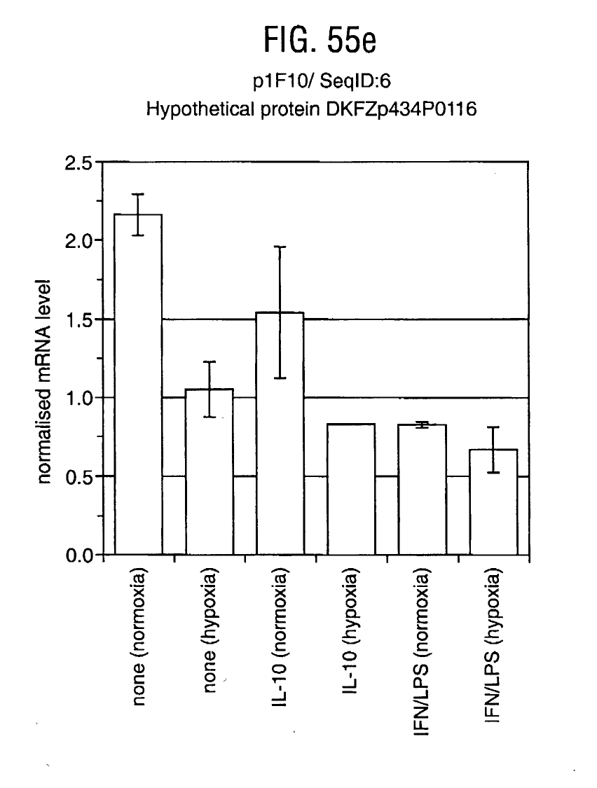

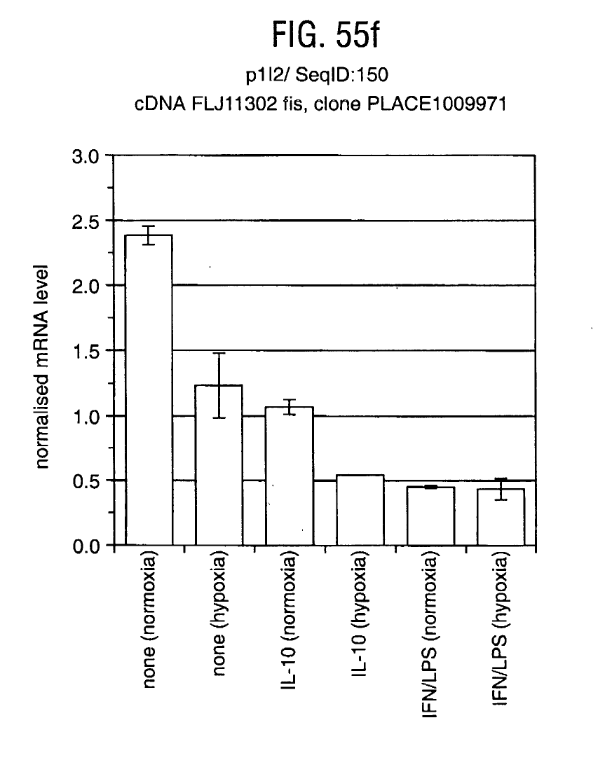

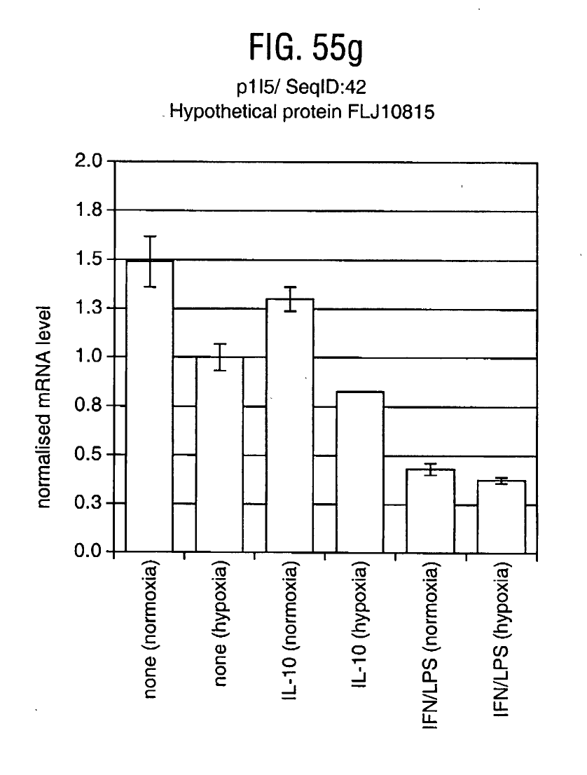

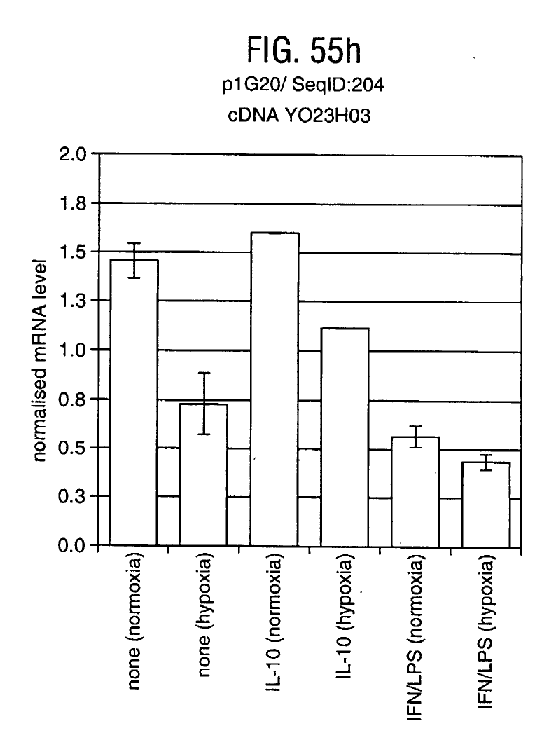

In FIG. 55, novel genes from Table 16 which are down-regulated both by LPS+IFN and by hypoxia are presented. In many of the genes presented here, these stimuli are synergistic, with minimal expression obtained with a combination of LPS+IFN and hypoxia.

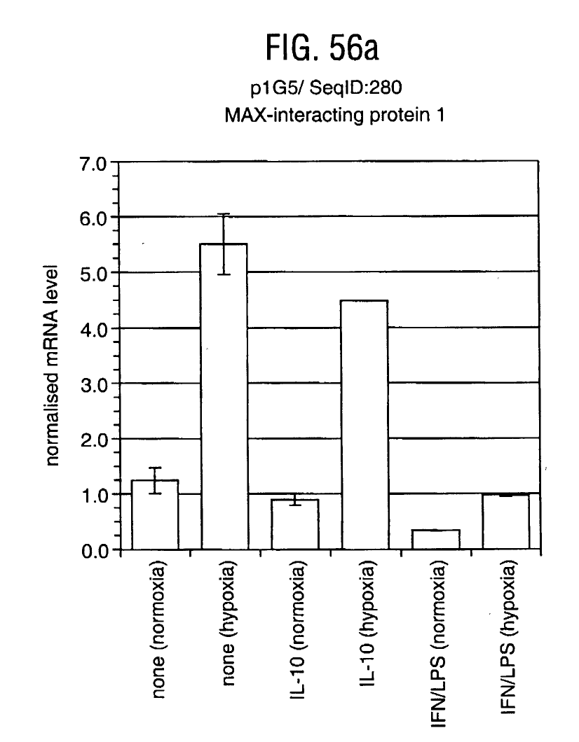

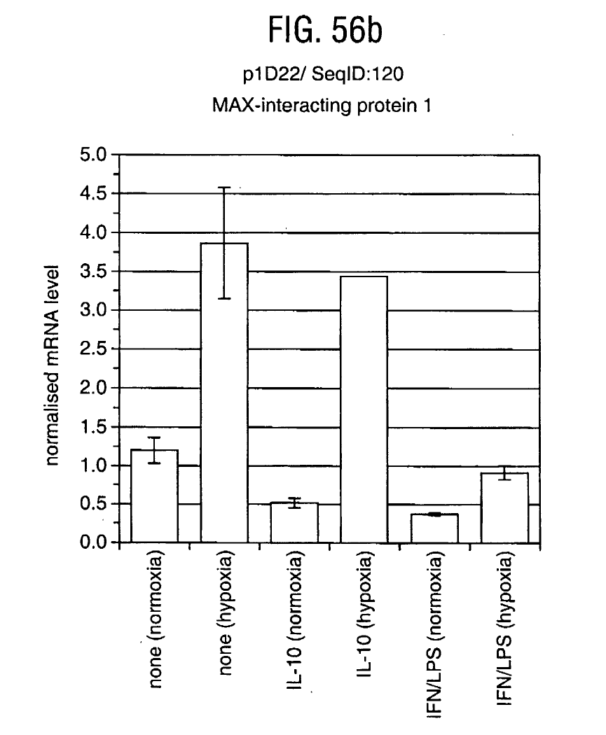

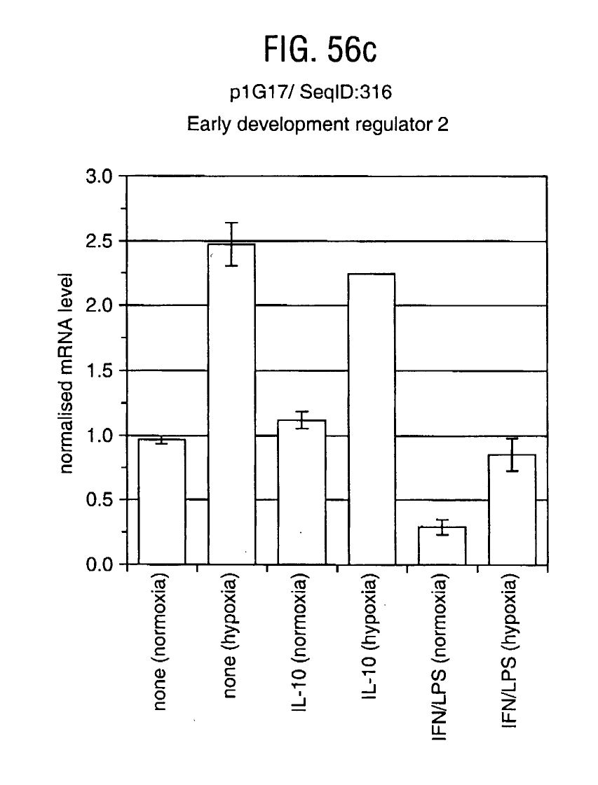

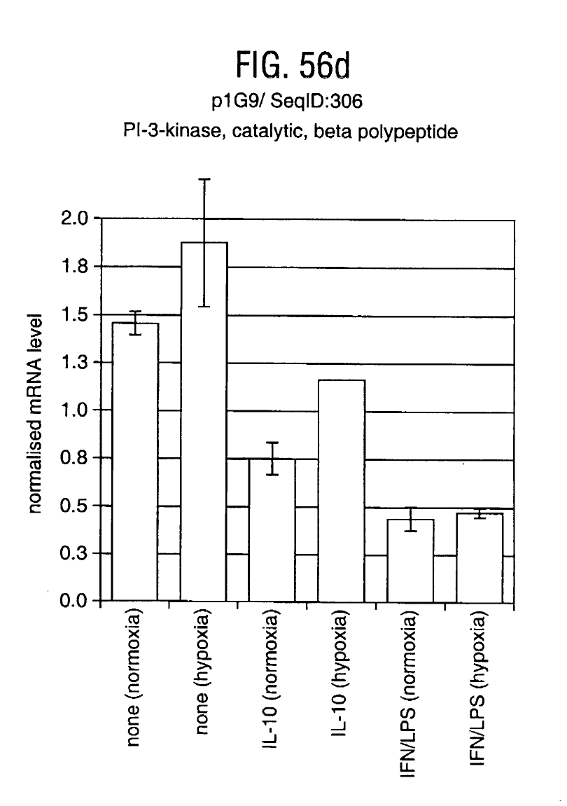

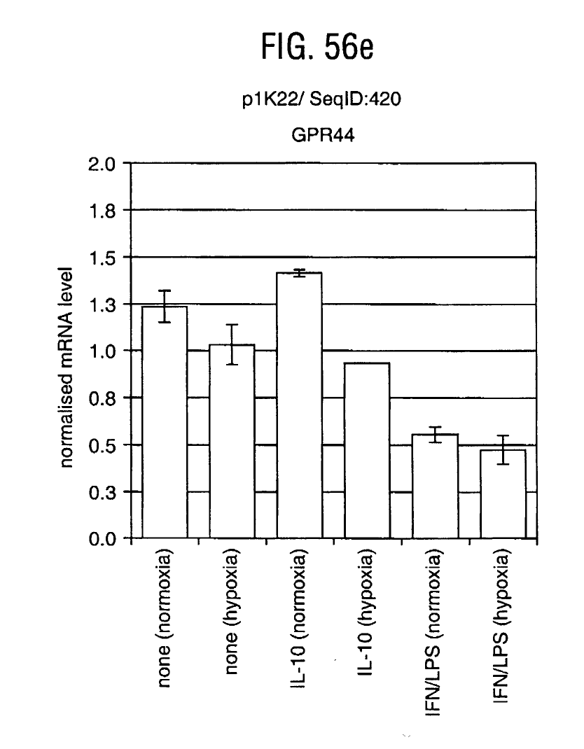

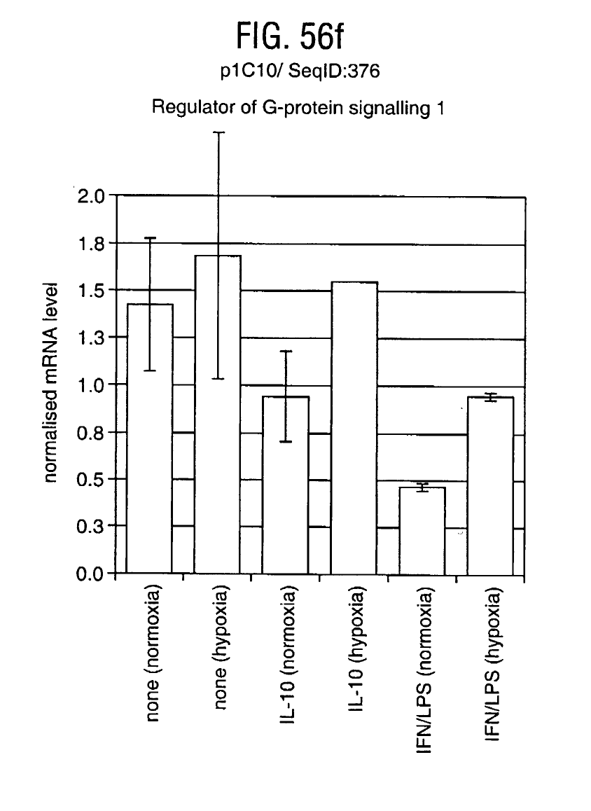

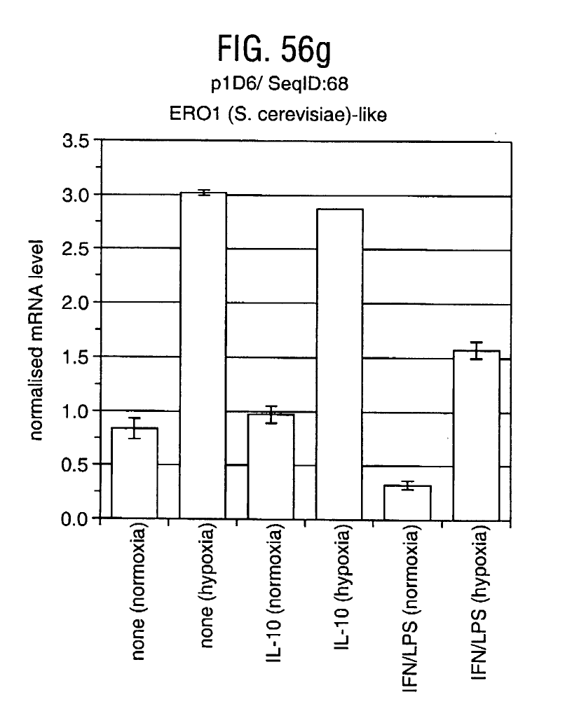

In FIG. 56, a selection of named genes from Table 16 which are down-regulated by LPS+IFN, with various responses to hypoxia are presented. For the gene, Max-interacting Protein 1 two separate clones were available on the array corresponding to this gene (p1G5 from SeqID:280 and p1D22 from SeqID:120). In the original specification, the IMAGE clone corresponding to SeqID:120 (accession AA401496) was classified as an EST, and the IMAGE clone corresponding to SeqID:280 (accession AA401496) was classified as “Max-interacting Protein 1”, as determined by the UniGene database at that time. Now it is apparent that both of these clones correspond to Max-interacting Protein 1, explaining the similarity of their expression profiles in FIG. 56. Clearly the response of this gene to hypoxia is inhibited by LPS+IFN.

The additional data showing effects of the Th1 activation stimulus LPS+IFN extends the finding of these genes as novel hypoxia regulated genes, and provides additional information about the relevance of these genes to disease mechanisms.

It will be appreciated that certain IMAGE clones were classed as novel and unannotated when the original patent filing was made (Dec. 8, 2000), but which can now be assigned to named genes. These are TRIP-Br2 (clone p1D15; SeqID:31/32), MAX-interacting protein 1 (clone p1D22; SeqID:119/120).

In Tables 15 and 16 and FIGS. 53-56, showing genes which respond to LPS+IFN, it will be noticed that some of these genes also response to the inhibitory cytokine IL-10 (e.g. Semaphorin 4b, hypothetical protein CGI-117). Other genes respond only to IL-10, but not to LPS+IFN. Specific responses to IL-10 are significant because this cytokine has been shown to have utility in suppressing inflammatory reactions (Huizinga T W et al., Rheumatology 2000, 39: 1180-8).

Table 17 shows genes responsive to IL-10 (increased or decreased) but not affected significantly by LPS+IFN. Various patterns of hypoxia regulation will be appreciated.

### Example 5

Gene Expression in Human Tumors

One of the utilities of the genes identified herein relates to the diagnosis and treatment of human tumors, on the basis that hypoxia is frequently found in tumors.

A study has been performed to examine the expression of these genes in a selection of breast and ovary tumors, comparing expression with normal adjacent tissue from the same patient. There is expected to be a large degree of variation between different patients, and the study here contains only 5 patients with a range of diagnoses. Therefore although certain genes will be identified from this data, other genes in the current specification not flagged by this study are nevertheless likely to have utility in cancer.

Patients are designated as Letters:

E: 50 year old Caucasian female. Diagnosis: ovarian adenocarcinoma. Normal ovarian tissue derived from an age-matched separate individual.

F: 60 year old female. Diagnosis: poorly differentiated adenocarcinoma. Normal ovarian tissue derived from the same individual.

G: 41 year old female. Diagnosis: moderately-differentiated adenocarcinoma. Normal ovarian tissue derived from the same individual.

H: 40 year old female. Diagnosis: invasive ductal carcinoma. Normnal breast tissue derived from the same individual.

K: 58 year old female. Diagnosis: invasive ductal carcinoma. Normal breast tissue derived from the same individual.

Data normalisation was done per-chip to correct for differences in labelling and hybridization efficiency. Per-gene normalisation was done such that the expression values of each gene are relative to the median value of that gene throughout the series of samples. By comparing the expression values under normal (nor) and tumor (turn) for a single patient, differences in expression between the normal and malignant tissue of that patient can be inferred.

In Table 18 are genes which are up-regulated at least 3-fold in at least one patient, comparing the tumor tissue to the adjacent normal tissue.

In Table 19 are genes which are down-regulated at least 3-fold in at least one patient, comparing the tumor tissue to the adjacent normal tissue.

### Example 6

Effects of Inflammatory Cytokines on Hypoxia-regulated Genes

Tumor necrosis factor alpha (TNFα) is a key pro-inflammatory cytokine both produced by and acting on the macrophage. The significance of TNFα to human disease is well established in the art. This is particularly the case in Rheumatoid arthritis and neutralising antibodies to TNFα have been reported to offer clinical utility. Because hypoxia is another pathological condition exerted on macrophages in the synovia of RA patients, synergistic effects of these two stimuli are highly relevant to the discovery of novel inflammatory targets expressed by the macrophage. To investigate this, primary human macrophages were exposed to either hypoxia (0.1% oxygen) or 100 ng/ml TNFα or to both for 6 hr. The data shown below provides further credence to the utility of the encoded proteins as inflammatory targets in macrophages and applies to any disease where hypoxia and TNFα are co-incident.

Gene expression levels were measured and compared using the custom gene array. In data analysis per-gene normalisation was set up such that expression values represent the fold-change compared with the expression in untreated normoxic cells. Genes which are increased in expression in response to TNFα by at least 2-fold, in either normoxic or hypoxic cells, are shown in Table 20. Genes which are decreased in expression in response to TNFα by at least 2-fold, in either normoxic or hypoxic cells, are shown in Table 21.

Another inflammatory cytokine implicated in diseases where hypoxia is frequently found is Interleukin-17 (IL-17). For example, this cytokine has been shown to mediate inflammation and joint destruction in arthritis (Lubberts et al J. Immunol 2001 167:1004-1013). IL-17 has also been shown to stimulate macrophages to release other key pro-inflammatory cytokines (Jovanovic et al J Immunol 1998 160:3513-21). Therefore genes which respond to both hypoxia and IL-17 are especially likely to be relevant to disease processes and have utility in the design of therapeutic products. Genes which are increased in expression in response to IL-17 by at least 2-fold, in either normoxic or hypoxic cells, are shown in Table 22. Genes which are decreased in expression in response to IL-17 by at least 2-fold, in either normoxic or hypoxic cells, are shown in Table 23.

The cytokine IL-15 is implicated in several disease in which macrophages and hypoxia both feature as elements of the inflammatory state, such as in atherosclerosis (Wuttge D M et al Am J Pathol. 2001 159:417-23) and rheumatoid arthritis (McInnes I B et al Immunol Today. 1998 19:75-9). Although the main target of IL-15 is T-cells effects have also been shown on monocytes (Badolato R et al Blood. 1997 90:2804-9). Therefore genes which respond to both hypoxia and IL-15 are especially likely to be relevant to disease processes and have utility in the design of therapeutic products. Genes which are increased in expression in response to IL-15 by at least 2-fold, in either normoxic or hypoxic cells, are shown in Table 24. Genes which are decreased in expression in response to IL-15 by at least 2-fold, in either normoxic or hypoxic cells, are shown in Table 25.

### Example 7

Rat Foetal Cardiomyocytes

Primary rat foetal cardiomyocytes provide an attractive experimental model for studying the responses of cardiac cells to ischaemia. Cells are obtained which are non-immortalized and which are seen to contract or beat in culture. It is of interest to examine how the responses of these cells to hypoxia (or related experimental conditions) compared and contrasts to other cell types. These other cell types might include those that are similarly sensitive to the effects of hypoxia (such as neurons) or might be cells that show a higher tolerance to hypoxia (such as macrophages). Experiments are performed in parallel for cardiomyocytes and other cell type(s). The responses of these specific cell types is then determined by hybridising labelled mRNA to microarrays. Alternative methods will include the construction of subtracted cDNA libraries for the individual treated cell types and assessing which genes are contained therein by sequencing.

Methods

Cardiomyocytes are harvested from heart ventricles of embryos aged E18 days, using a cell isolation kit (Neonatal cardiomyocyte isolation system; Worthington Biochemical Corporation, Lakewood, N.J. 08701). They are seeded at 5×10⁶ cells/100 cm diameter petri dish in DMEM/M199, 10% horse serum, 5% FCS, 1% penicillin, streptomycin, glutamine for 5 days at 37 C. Media is changed during the 5 days.

Other cell types used for comparison with cardiomyocytes, are cultured according to their optimum conditions and/or the standard routine. These cell types may include cardiomyocytes in a different physiological setting, such as in an intact beating heart, or a different developmental state of the cardiomyocyte, such as cardiomyoblast.

Identical seeded petri dishes are placed either in a standard tissue culture incubator (95% air/5% CO2) or in a hypoxia incubator (0.1% oxygen/5% CO2/0.1% oxygen for 6 hours. This is done separately for both cardiomyocytes and the other cell type(s) to be compared. Other experimental conditions might more closely approximate ishemia, by incorporating components additional to hypoxia.

At the end of the exposure to hypoxia, cells are placed on a chilled platform, washed in cold PBS and total RNA is extracted using RNazol B (Tel-Test, Inc; distributed by Biogenesis Ltd) following the manufacturers instructions. Where appropriate, polyadenylated mRNA is extracted from the total RNA using a commercial kit following the manufacturers instructions (Promega; PolyATract mRNA isolation System IV).

Array hybridizations and construction/analysis of subtracted cDNA libraries are performed according to standard methods or as described elsewhere in this specification.

### Example 8

Comparison of the Hypoxic-responses Between Populations of Rat Primary Cultured Neurons by a Subtraction Cloning/Array Screening Approach.

Different regions of the central nervous system display different sensitivities to hypoxia and to ischaemia. Susceptibility to tissue damage in this manner may occur as a result of intrinsic differences in gene expression between cells. To evaluate this hypothesis, primary cultures of rat neurons from different regions of the brain are established. Cultures are exposed to various experimental conditions which are pertinent to pathologies of the hypoxic/ischemic brain. These would include hypoxic insults as have been described, or to hypoxia/ischaemia where the conditions more closely approximate pathological ischemia. Either condition may be preceded by prior hypoxic-preconditioning, where transient exposure to hypoxia renders cells less sensitive to subsequent acute treatment. For all possible experimental treatments, a similar routine is performed for distinct neuron subtypes, in order to compare their responses. Such comparisons may be made by hybridizing labelled mRNA to microarrays or derivatives thereof. Alternatively subtracted libraries might be constructed individually for each treated neuron subtype, and clones which are confirmed to be changed in expression to be sequenced. The collection of genes arising from the different neuron subtypes will be compared.

Methods

Primary cultures are established according to standard procedures from embryonic rats aged from E14 to E18 (Dunnett S B, Bjorkland A (Eds.) 1992. Neural Transplantation, A Practical Approach. IRL Press). Isolated neurons include but are not limited to those from ventral mesencephalon, striatum, hippocampus, cerebellum, cerebral cortex, dorsal root ganglia and superior cervical ganglia.

Cells are maintained in culture for 3-14 days in humidified culture incubators at 37° C., 5% CO2, 95% air (Normoxia) in Neurobasal Medium (Brewer G J, 1995, Journal of Neuroscience Research 42:674-83) supplemented with B27 (both Life Technologies). For the hypoxia-preconditioning, cells are transferred to a second incubator at 37° C., 5% CO2, 94.9% Nitrogen, 0.1% Oxygen (Hypoxia) for 30-180 minutes and returned to the normoxic incubator for 24 hours (Pringle et al., 1997, Neuropathology and Applied Neurobiology 23:289-298). For the hypoxic stimulus, either independent from or subsequent to hypoxia-preconditioning, cells are transferred to the hypoxic incubator for 2-6 hours as determined in time course experiments. Additionally, as appropriate, the medium in which the cells are grown is replaced with glucose-free media for establishment of experimental ischaemia (Ray A M, Owen D E, Evans M L, Davis J B Benham, 2000. Caspase inhibitors are functionally neuroprotective against oxygen glucose deprivation induced CA1 death in rat organotypic hippocampal slices). At the end of the exposure to hypoxia (or hypoxia/ischaemia), cells are, placed on a chilled platform, washed in cold PBS and total RNA is extracted using RNazol B (Tel-Test, Inc; distributed by Biogenesis Ltd) following the manufacturers instructions. Where appropriate, polyadenylated mRNA is extracted from the total RNA using a commercial kit following the manufacturers instructions (Promega; PolyATract mRNA isolation System IV).

Array hybridizations and construction/analysis of subtracted cDNA libraries are performed according to standard methods or as described elsewhere in this specification.

### Example 9

Semaphorin 4b

cDNA libraries derived from the human brain and leukocytes were removed to obtain an unequivocal and accurate full length cDNA sequence (SEQ ID No 92a) and the accurate presumptive amino acid sequence (SEQ ID No 91).

The amino acid sequence above was derived by taking the first ATG. We have various independent lines of evidence that this is the bonafide translation initiation codon.

Basic analysis of this sequence, reveals the following motifs:

| | | |
|---|---|---|
| signal peptide (pSORT) | Start: 1 | End: 37; |
| Transmembrane (pSORT) | Start: 718 | End: 734; |
| cleavage site (pSORT) | Start: 38 | End: 38; |
| Proline rich region | Start: 758 | End: 824; |
| Sema domain (pfam) | Start: 70 | End: 503; |
| Plexin repeat (pfam) | Start: 70 | End: 548; |
| integrin, beta domain (pfam) | Start: 532 | End: 546; |
| cytoplasmic tail | Start: 735 | End: 837. |

To confirm the hypoxic regulation of Sema4b, we used RNase protection assay (see FIG. 57). Hypoxia is a feature of several inflammatory conditions often accompanied by superoxide radicals and the immune regulator gamma interferon. In this experiment we have made the following findings:

Expression is activated by hypoxia (3.3 fold)

Expression is activated by gamma interferon and LPS (3.9 fold)

Expression is activated synergistically by hypoxia plus gamma interferon/LPS (7.3 fold)

Expression is activated by superoxide radicals (5.0 fold)

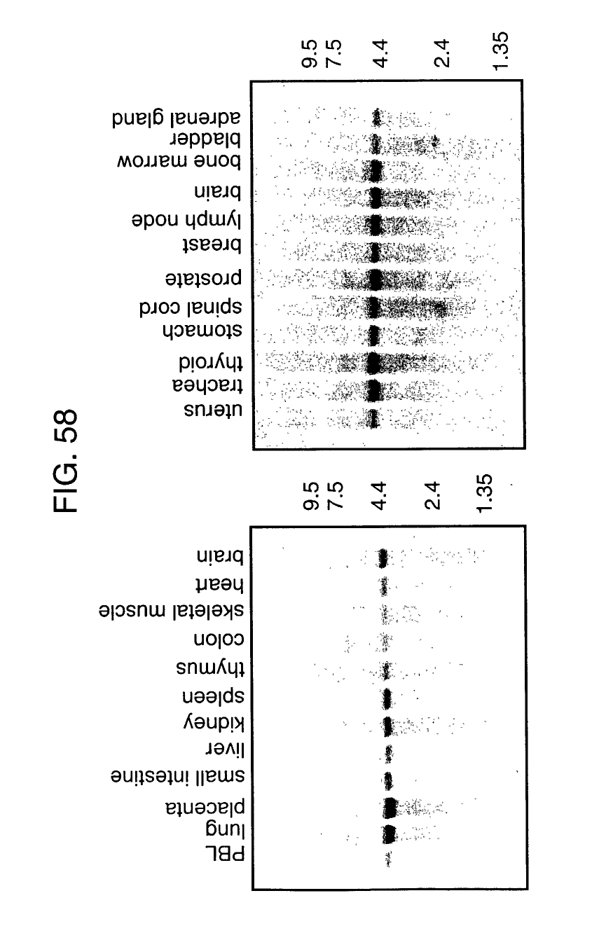

To investigate the size of the mRNA and the tissue distribution, Northern blotting was done (see FIG. 58). This shows that the gene is expressed as a single transcript at relatively low levels in unstimulated human tissues.

We have also found that a molecule that is probably associated with Semaphorin 4B, called psd-95 is another macrophage hypoxia-induced protein (see SEQ ID No 299). This is based on the fact that psd-95 binds the cytoplasmic tail of Sema4c (Inagaki et al., J Biol Chem. 2001; 276(12): 9174-81), which like Sema4b, contains proline rich sequence. Therefore, both Semaphorin 4B, and a probable partner are co-ordinately regulated by hypoxia.

### Example 10

Discussion of Relevance of Individual Clones

Clone p1F12 represents hypothetical protein FLJ13611. The protein sequence encoded by hypothetical protein FLJ13611 is represented in the public databases by the accession NP_{—}079217 and is described in this patent by SEQ ID NO: 1. The nucleotide sequence is represented in the public sequence databases by the accession NM_{—}024941 and is described in this patent by SEQ ID NO: 2. Hypoxia is an important feature of several diseases, and genes that respond to this stimulus are therefore implicated in the pathogenesis and have utility in the design of therapeutic, prognostic and diagnostic products.

Clone p1F2 represents hypothetical protein FLJ20037. The protein sequence encoded by hypothetical protein FLJ20037 is represented in the public databases by the accession CAB65981 and is described in this patent by SEQ ID NO: 3. The nucleotide sequence is represented in the public sequence databases by the accession NM_{—}017633 and is described in this patent by SEQ ID NO: 4. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, hypothetical protein FLJ20037 is down-regulated in the malignant tissue as compared to adjacent normal tissue in at least one patient. We expect increased activity of the gene product to have an anti-tumour effect.

Clone p1F10 represents hypothetical protein DKFZp434P0116. The protein sequence encoded by hypothetical protein DKFZp434P0116 is represented in the public databases by the accession T46364 and is described in this patent by SEQ ID NO: 5. The nucleotide sequence is represented in the public sequence databases by the accession NM_{—}017593 and is described in this patent by SEQ ID NO: 6. Hypothetical protein DKFZp434P0116 is predicted to be a kinase due to high structural similarity with other known kinases. Hypoxia is an important feature of several diseases, and genes that respond to this stimulus are therefore implicated in the pathogenesis and have utility in the design of therapeutic, prognostic and diagnostic products. Hypothetical protein DKFZp434P0116 is repressed in macrophages activated by LPS and gamma interferon. We expect it to have an anti-inflammatory role.

Clone p1F19 represents hypothetical protein KIAA0212. The protein sequence encoded by hypothetical protein KIAA0212 is represented in the public databases by the accession BAA13203 and is described in this patent by SEQ ID NO: 7. The nucleotide sequence is represented in the public sequence databases by the accession NM_{—}014674 and is described in this patent by SEQ ID NO: 8. Hypoxia is an important feature of several diseases, and genes that respond to this stimulus are therefore implicated in the pathogenesis and have utility in the design of therapeutic, prognostic and diagnostic products.

Clone p1F8 represents hypothetical protein KIAA0914. The protein sequence encoded by hypothetical protein KIAA0914 is represented in the public databases by the accession NP_{—}055698 and is described in this patent by SEQ ID NO: 9. The nucleotide sequence is represented in the public sequence databases by the accession NM_{—}014883 and is described in this patent by SEQ ID NO: 10. Hypothetical protein KIAA0914 shows high structural similarity to Human Class I alpha 1,2-Mannosidase and conservation of active site and binding site residues, therefore we predict that hypothetical protein KIAA0914 will act as a mannosidase. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein KIAA0914 is repressed in macrophages activated by LPS and gamma interferon. We expect the gene product to have an anti-inflammatory role.

Clone p1F5 represents hypothetical protein FLJ20281. The protein sequence encoded by hypothetical protein FLJ20281 is represented in the public databases by the accession XP_{—}008736 and is described in this patent by SEQ ID NO: 11. The nucleotide sequence is represented in the public sequence databases by the accession NM_{—}017742 and is described in this patent by SEQ ID NO: 12. Hypoxia is an important feature of several diseases, and genes that respond to this stimulus are therefore implicated in the pathogenesis and have utility in the design of therapeutic, prognostic and diagnostic products. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Hypothetical protein FLJ20281 is induced in macrophages activated by TNFalpha.

Clone p1F18 represents hypothetical protein KIAA0876. The protein sequence encoded by hypothetical protein KIAA0876 is represented in the public databases by the accession BAA74899 and is described in this patent by SEQ ID NO: 13. The nucleotide sequence is represented in the public sequence databases by the accession XM_{—}035625 and is described in this patent by SEQ ID NO: 14. Hypoxia is an important feature of several diseases, and genes that respond to this stimulus are therefore implicated in the pathogenesis and have utility in the design of therapeutic, prognostic and diagnostic products.

Clone p1F7 represents Spectrin, beta, non-erythrocytic 1. The protein sequence encoded by Spectrin, beta, non-erythrocytic 1 is represented in the public databases by the accession NP_{—}003119 and is described in this patent by SEQ ID NO: 15. The nucleotide sequence is represented in the public sequence databases by the accession NM_{—}003128 and is described in this patent by SEQ ID NO: 16. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, Spectrin, beta, non-erythrocytic 1 is down-regulated in the malignant tissue as compared to adjacent normal tissue in at least one patient. We expect increased activity of the gene product to have an anti-tumour effect.

Clone p1F21 represents Hematopoietic Zinc finger protein. The protein sequence encoded by Hematopoietic Zinc finger protein is represented in the public databases by the accession AAL08625 and is described in this patent by SEQ ID NO: 17. The nucleotide sequence is represented in the public sequence databases by the accession AK024404 and is described in this patent by SEQ ID NO: 18. Hematopoietic Zinc finger protein is a transcriptional regulator that contains a Cys2-His2 zinc finger motif. It is predicted to bind to metal response elements (MRE) and therefore activate the transcription of genes that contain a MRE sequence within their promoter region such as metallothioneins. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Hematopoietic Zinc finger protein is preferentially induced by hypoxia in monocytes or macrophages and a restricted number of other cell types. It is therefore a candidate for specific intervention for treatment or diagnosis of the above diseases.

Clone p1F9 represents hypothetical protein KIAA0742. The protein sequence encoded by hypothetical protein KIAA0742 is represented in the public databases by the accession NP_{—}060903 and is described in this patent by SEQ ID NO: 19. The nucleotide sequence is represented in the public sequence databases by the accession AB018285 and is described in this patent by SEQ ID NO: 20. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein KIAA0742 is repressed in macrophages activated by LPS and gamma interferon. We expect it to have an anti-inflammatory role. Hypothetical protein KIAA0742 shows significant homolgy to the transcription factor hairless. We therefore propose that hypothetical protein KIAA0742 may play a crucial role in the regulation of hair growth. Accordingly, this aspect of the invention includes the use of this protein, fragments and functional equivalents of this protein, encoding nucleic acid molecules, in addition to ligands that bind specifically to this protein, in the diagnosis and treatment of hair loss.

Clone p1E13 represents hypothetical protein PRO0823. The protein sequence encoded by hypothetical protein PRO0823 is represented in the public databases by the accession AAF71073 and is described in this patent by SEQ ID NO: 21. The nucleotide sequence is represented in the public sequence databases by the accession AF116653 and is described in this patent by SEQ ID NO: 22. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein PRO0823 is repressed in macrophages activated by LPS and gamma interferon. We expect it to have an anti-inflammatory role. Hypoxia is frequently found in human tumours where macrophage infiltrates are also found. In a series of 5 patients with either ovarian or breast cancer, hypothetical protein PRO0823 is down-regulated in the malignant tissue as compared to adjacent normal tissue in at least one patient. We expect increased activity of the gene product to have an anti-tumour effect.

Clones p1D1 and p1D2 represent the hypothetical protein FLJ10134. The protein sequence encoded by hypothetical protein FLJ10134 is represented in the public databases by the accession NP_{—}060474 and is described in this patent by SEQ ID NO: 23. The nucleotide sequence is represented in the public sequence databases by the accession NM_{—}018004 and is described in this patent by SEQ ID NO: 24. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors that mediate the response to hypoxia of several genes, and have them selves been implicated in specific diseases. By adenoviral over-expression of EPAS1, we show augmentation of the hypoxic induction of certain genes, further confirming their status as responsive to hypoxia. Hypothetical protein FLJ10134 has been shown to be induced by hypoxia to a greater degree following adenoviral over-expression of EPAS1. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein FLJ101134 is repressed in macrophages activated by LPS and gamma interferon. We expect it to have an anti-inflammatory role. Hypoxia is frequently found in human tumours where macrophage infiltrates are also found. In a series of 5 patients with either ovarian or breast cancer, hypothetical protein FLJ101134 is down-regulated in the malignant tissue as compared to adjacent normal tissue in at least one patient. We expect increased activity of the gene product to have an anti-tumour effect.

Clone p1D4 represents hypothetical protein FLJ20500. The protein sequence encoded by hypothetical protein FLJ20500 is represented in the public databases by the accession NP_{—}061931 and is described in this patent by SEQ ID NO: 25. The nucleotide sequence is represented in the public sequence databases by the accession NM_{—}019058 and is described in this patent by SEQ ID NO: 26. Hypoxia is an important feature of several diseases, and genes that respond to this stimulus are therefore implicated in the pathogenesis and have utility in the design of therapeutic, prognostic and diagnostic products. Hypothetical protein FLJ20500 is preferentially induced by hypoxia in mammary epithelial cells.

Clone p1D9 represents hypothetical protein DKFZP564D116. The protein sequence encoded by hypothetical protein DKFZP564D116 is represented in the public databases by the accession T08708 and is described in this patent by SEQ ID NO: 27. The nucleotide sequence is represented in the public sequence databases by the accession AL050022 and is described in this patent by SEQ ID NO: 28. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein DKFZP564D116 is repressed in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Hypothetical protein DKFZP564D116 is induced in macrophages activated by TNFalpha.

Clone p1D12 represents hypothetical protein KIAA1376. The protein sequence encoded by hypothetical protein KIAA1376 is represented in the public databases by the accession BAA92614 and is described in this patent by SEQ ID NO: 29. The nucleotide sequence is represented in the public sequence databases by the accession AB037797 and is described in this patent by SEQ ID NO: 30. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein KIAA1376 is repressed in macrophages activated by LPS and gamma interferon. We expect it to have an anti-inflammatory role.

Clone p1D15 represents TRIP-Br2. The protein sequence encoded by TRIP-Br2 is represented in the public databases by the accession NP_{—}055570 and is described in this patent by SEQ ID NO: 31. The nucleotide sequence is represented in the public sequence databases by the accession NM_{—}014755 and is described in this patent by SEQ ID NO: 32.. TRIP-BR2 is a PHD zinc finger and bromodomain interacting protein transcriptional regulator and is involved in the regulation of cell cycle progression. Its hypoxia-regulation is likely to have important disease-relevant effects. Hypoxia is an important feature of several diseases, and genes that respond to this stimulus are therefore implicated in the pathogenesis and have utility in the design of therapeutic, prognostic and diagnostic products. The response of renal epithelial cells to hypoxia is pertinent to kidney failure, especially regarding the medullary tissue. TRIP-Br2 is preferentially induced by hypoxia in renal epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TRIP-Br2 is repressed in macrophages activated by LPS and gamma interferon.

Clone p1D16 represents hypothetical protein FLJ20308. The protein sequence encoded by hypothetical protein FLJ20308 is represented in the public databases by the accession XP_{—}039852 and is described in this patent by SEQ ID NO: 33. The nucleotide sequence is represented in the public sequence databases by the accession AK000315 and is described in this patent by SEQ ID NO: 34. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein FLJ20308 is repressed in macrophages activated by LPS and gamma interferon. We expect it to have an anti-inflammatory role.

Clone p1J13 represents hypothetical nuclear factor SBBI22. The protein sequence encoded by hypothetical nuclear factor SBBI22 is represented in the public databases by the accession NP_{—}065128 and is described in this patent by SEQ ID NO: 35. The nucleotide sequence is represented in the public sequence databases by the accession NM_{—}020395 and is described in this patent by SEQ ID NO: 36. Hypoxia is an important feature of several diseases, and genes that respond to this stimulus are therefore implicated in the pathogenesis and have utility in the design of therapeutic, prognostic and diagnostic products.

Clone p1I22 represents hypothetical protein KIAA1429. The protein sequence encoded by hypothetical protein KIAA1429 is represented in the public databases by the accession BAA92667 and is described in this patent by SEQ ID NO: 37. The nucleotide sequence is represented in the public sequence databases by the accession AB037850 and is described in this patent by SEQ ID NO: 38. Hypoxia is an important feature of several diseases, and genes that respond to this stimulus are therefore implicated in the pathogenesis and have utility in the design of therapeutic, prognostic and diagnostic products.

Clone p1J6 represents hypothetical protein FLJ10206. The protein sequence encoded by hypothetical protein FLJ10206 is represented in the public databases by the accession AAH06108 and is described in this patent by SEQ ID NO: 39. The nucleotide sequence is represented in the public sequence databases by the accession NM_{—}018025 and is described in this patent by SEQ ID NO: 40. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein FLJ10206 is repressed in macrophages activated by IL-17 and is also repressed in macrophages activated by IL-15. These are pro-inflammatory cytokines, and we expect the hypothetical protein FLJ10206 to have an anti-inflammatory role.

Clone p1I5 represents hypothetical protein FLJ10815. The protein sequence encoded by hypothetical protein FLJ10815 is represented in the public databases by the accession BAA91830 and is described in this patent by SEQ ID NO: 41. The nucleotide sequence is represented in the public sequence databases by the accession NM_{—}018231 and is described in this patent by SEQ ID NO: 42. Hypothetical protein FLJ10815 is structurally similar to an alpha/beta barrel structure. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein FLJ10815 is repressed in macrophages activated by LPS and gamma interferon. We expect it to have an anti-inflammatory role.

Clone p1I13 represents hypothetical protein FWJ11100. The protein sequence encoded by hypothetical protein FLJ11100 is represented in the public databases by the accession NP_{—}060701 and is described in this patent by SEQ ID NO: 43. The nucleotide sequence is represented in the public sequence databases by the accession NM_{—}018321 and is described in this patent by SEQ ID NO: 44. Hypoxia is an important feature of several diseases, and genes that respond to this stimulus are therefore implicated in the pathogenesis and have utility in the design of therapeutic, prognostic and diagnostic products.

Clone p1I17 represents hypothetical protein FLJ20644. The protein sequence encoded by hypothetical protein FLJ20644 is represented in the public databases by the accession NP_{—}060387 and is described in this patent by SEQ.ID NO: 45. Hypothetical protein FLJ20644 is a putative Serine/threonine phosphotase. Region 250-450 shows high structural similarity to other Serine/threonine phosphotases. The nucleotide sequence is represented in the public sequence databases by the accession NM_{—}017917 and is described in this patent by SEQ ID NO: 46. Hypoxia is an important feature of several diseases, and genes that respond to this stimulus are therefore implicated in the pathogenesis and have utility in the design of therapeutic, prognostic and diagnostic products.

Clone p1I15 represents hypothetical protein CGI-117. The protein sequence encoded by hypothetical protein CGI-117 is represented in the public databases by the accession Q9Y3C1 and is described in this patent by SEQ ID NO: 47. The nucleotide sequence is represented in the public sequence databases by the accession NM_{—}016391 and is described in this patent by SEQ ID NO: 48. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors that mediate the response to hypoxia of several genes, and have them selves been implicated in specific diseases. By adenoviral over-expression of HIF1alpha or EPAS1 we show augmentation of the hypoxic induction of certain genes, further confirming their status as responsive to hypoxia. Hypothetical protein CGI-117 has been shown to be induced by hypoxia to a greater degree following adenoviral over-expression of either HIF1alpha or EPAS1. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein CGI-117 is repressed in macrophages activated by LPS and gamma interferon. We expect it to have an anti-inflammatory role.

Clone p1I7 represents Uridine 5′ monophosphate hydrolase 1. The protein sequence encoded by Uridine 5′ monophosphate hydrolase 1 is represented in the public databases by the accession NP_{—}057573 and is described in this patent by SEQ ID NO: 49. The nucleotide sequence is represented in the public sequence databases by the accession NM_{—}016489 and is described in this patent by SEQ ID NO: 50. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Uridine 5′ monophosphate hydrolase 1 is induced in macrophages activated by LPS and gamma interferon and is also is induced in macrophages activated by IL-15. We expect it to have a pro-inflammatory role, and its inhibition may have an anti-inflammatory effect.

The protein sequence encoded by hypothetical protein KIAA0014 is represented in the public databases by the accession NP_{—}055480 and is described in this patent by SEQ ID NO: 51. The nucleotide sequence is represented in the public sequence databases by the accession NM_{—}014665 and is described in this patent by SEQ ID NO: 52. Hypoxia is an important feature of several diseases, and genes that respond to this stimulus are therefore implicated in the pathogenesis and have utility in the design of therapeutic, prognostic and diagnostic products.

Clone p1I4 represents hypothetical protein HSPC196. The protein sequence encoded by hypothetical protein HSPC196 is represented in the public databases by the accession NP_{—}057548 and is described in this patent by SEQ ID NO: 53. The nucleotide sequence is represented in the public sequence databases by the accession NM_{—}016464 and is described in this patent by SEQ ID NO: 54. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein HSPC196 is repressed in macrophages activated by LPS and gamma interferon. We expect it to have an anti-inflammatory role.

Clone p1I8 represents hypothetical protein FLJ11296. The protein sequence encoded by hypothetical protein FLJ11296 is represented in the public databases by the accession XP_{—}004747 and is described in this patent by SEQ ID NO: 55. The nucleotide sequence is represented in the public sequence databases by the accession NM_{—}018384 and is described in this patent by SEQ ID NO: 56. Hypoxia is an important feature of several diseases, and genes that respond to this stimulus are therefore implicated in the pathogenesis and have utility in the design of therapeutic, prognostic and diagnostic products.

Clone p1I16 represents hypothetical protein KLAA1668. The protein sequence encoded by hypothetical protein KIAA1668 is represented in the public databases by the accession BAB33338 and is described in this patent by SEQ ID NO: 57. The nucleotide sequence is represented in the public sequence databases by the accession AB051455 and is described in this patent by SEQ ID NO: 58. Hypoxia is an important feature of several diseases, and genes that respond to this stimulus are therefore implicated in the pathogenesis and have utility in the design of therapeutic, prognostic and diagnostic products.

Clone p1I11represents SECIS binding protein 2. The protein sequence encoded by SECIS binding protein 2 is represented in the public databases by the accession AAK57518 and is described in this patent by SEQ ID NO: 59. The nucleotide sequence is represented in the public sequence databases by the accession AF380995 and is described in this patent by SEQ ID NO: 60. SECIS binding protein 2 is a crucial component in the complex required for the translation of mammalian selenoprotein mRNAs. Selenoproteins are important responders to redox conditions and many selenoproteins are known to protect from cell death. Our demonstration of the hypoxia induction of SECIS binding protein 2 opens new avenues for diagnosis and therapeutic intervention. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, SECIS binding protein 2 is down-regulated in the malignant tissue as compared to adjacent normal tissue in at least one patient. We expect increased activity of the gene product to have an anti-tumour effect.

Clone p1E8 represents cDNA: FLJ22249 fis, clone HRC02674. The sequence cDNA: FLJ22249 fis, clone HRC02674 is not represented in the public databases by a protein accession. The nucleotide sequence is represented in the public sequence databases by the accession AK025902 and is described in this patent by SEQ ID NO: 62. Hypoxia is an important feature of several diseases, and genes that respond to this stimulus are therefore implicated in the pathogenesis and have utility in the design of therapeutic, prognostic and diagnostic products.

Clone p1E18 represents Plexin C1. The protein sequence encoded by Plexin C1 is represented in the public databases by the accession NP_{—}005752 and is described in this patent by SEQ ID NO: 63. The nucleotide sequence is represented in the public sequence databases by the accession NM_{—}005761 and is described in this patent by SEQ ID NO: 64. Plexins are a large family of receptors for transmembrane, secreted, and GPI-anchored semaphorins in vertebrates and play a significant role in signal transduction [Tamagnone et al 1999, Cell 99:71-80]. Elsewhere in this patent we disclose hypoxic regulation of a new semaphorin 4b, and we propose co-regulation of these molecules by hypoxia and their relevance to inflammatory disease, its diagnosis and therapy. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Plexin C1 is repressed in macrophages activated by LPS and gamma interferon.

Clone p1E16 represents cDNA DKFZp586E1624. The sequence cDNA DKFZp586E1624 is not represented in the public databases by a protein accession. The nucleotide sequence is represented in the public sequence databases by the accession AL110152 and is described in this patent by SEQ ID NO: 66. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors that mediate the response to hypoxia of several genes, and have them selves been implicated in specific diseases. By adenoviral over-expression of EPAS1 we show augmentation of the hypoxic induction of certain genes, further confirming their status as responsive to hypoxia. Its preferential regulation by EPAS1 provides a route to preferential intervention, to avoid toxicity to other tissues. The cDNA DKFZp586E1624 has been shown to be induced by hypoxia to a greater degree following adenoviral over-expression of EPAS1. Endothelial cells are key to angiogenesis, a process implicated in several diseases associated with hypoxia, including cancer and rheumatoid arthritis. The CDNA DKFZp586E1624 is preferentially induced by hypoxia in endothelial cells. We expect this gene product to have a pro-angiogenic effect, and its inhibition to have an anti-angiogenic effect.

Clones p1D5 and p1D6 represent ERO1 (S. cerevisiae)-like. The protein sequence encoded by ERO1 (S. cerevisiae)-like is represented in the public databases by the accession NP_{—}055399 and is described in this patent by SEQ ID NO: 67. The nucleotide sequence is represented in the public sequence databases by the accession NM_{—}014584 and is described in this patent by SEQ ID NO: 68. ERO1 (S. cerevisiae)-like has been shown to be a flavin adenine dinucleotide (FAD) binding protein. Binding of FAD enables ERO1 (S. cerevisiae)-like to oxidise protein disulfide isomerase (PDI). We propose that the oxidisation of PDI by ERO1 (S. cerevisiae)-like stops PDI autodegradation, therefore increasing levels of the protein. Increased levels of PDI have been shown to be neuroprotective by inhibiting apoptotic cell death. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors that mediate the response to hypoxia of several genes, and have them selves been implicated in specific diseases. By adenoviral over-expression of EPAS1 we show augmentation of the hypoxic induction of certain genes, further confirming their status as responsive to hypoxia. ERO1 (S. cerevisiae)-like has been shown to be induced by hypoxia to a greater degree following adenoviral over-expression of EPAS1. Its preferential regulation by EPAS1 provides a route to preferential intervention, to avoid toxicity to other tissues. ERO1 (S. cerevisiae)-like is preferentially induced by hypoxia in mammary epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. ERO1 (S. cerevisiae)-like is repressed in macrophages activated by LPS and gamma interferon. Hypoxia is frequently found in human tumours where macrophage infiltrates are also found. In a series of 5 patients with either ovarian or breast cancer, ERO1 (S. cerevisiae)-like is up-regulated in the malignant tissue as compared to adjacent normal tissue in at least one patient.

Clone p1E12 represents hypothetical protein DKFZP434E1723. The protein sequence encoded by hypothetical protein DKFZP434E1723 is represented in the public databases by the accession XP_{—}05338 and is described in this patent by SEQ ID NO: 69. The nucleotide sequence is represented in the public sequence databases by the accession BC010005 and is described in this patent by SEQ ID NO: 70. Hypoxia is an important feature of several diseases, and genes that respond to this stimulus are therefore implicated in the pathogenesis and have utility in the design of therapeutic, prognostic and diagnostic products.

Clone p1E10 represents cDNA FLJ11041 fis clone PLACE1004405. The sequence encoded by cDNA FLJ11041 fis, clone PLACE1004405 is not represented in the public databases by a protein accession. The nucleotide sequence is represented in the public sequence databases by the accession AK001903 and is described in this patent by SEQ ID NO: 72. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The cDNA FLJ11041 fis clone PLACE1004405 is induced in macrophages activated by LPS and gamma interferon. We expect it to have a pro-inflammatory role, and its inhibition may have an anti-inflammatory effect.

Clone p1C21 represents Tubulin, beta, 4. The protein sequence encoded by Tubulin, beta, 4 is represented in the public databases by the accession NP_{—}006077 and is described in this patent by SEQ ID NO: 73. The nucleotide sequence is represented in the public sequence databases by the accession NM_{—}006086 and is described in this patent by SEQ ID NO: 74. Hypoxia is an important feature of several diseases, and genes that respond to this stimulus are therefore implicated in the pathogenesis and have utility in the design of therapeutic, prognostic and diagnostic products.

Clone p1D10 represents Insulin induced protein 2. The protein sequence encoded by Insulin induced protein 2 is represented in the public databases by the accession AAD43048 and is described in this patent by SEQ ID NO: 75. The nucleotide sequence is represented in the public sequence databases by the accession AF125392 and is described in this patent by SEQ ID NO: 76. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Insulin induced protein 2 is induced in macrophages activated by LPS and gamma interferon. We expect it to have a pro-inflammatory role, and its inhibition may have an anti-inflammatory effect.

Clones p1D13 and p1A22 represent Adenylate kinase 3. The protein sequence encoded by Adenylate kinase 3 is represented in the public databases by the accession NP_{—}037542 and is described in this patent by SEQ ID NO: 77 and 263. The nucleotide sequence is represented in the public sequence databases by the accession NM_{—}013410 and is described in this patent by SEQ ID NO: 78 and 264. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Adenylate kinase 3 is induced in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Adenylate kinase 3 is induced in macrophages activated by TNFalpha.

Clone p1E9 represents a novel PI-3-kinase adapter. The protein sequence encoded by the novel PI-3-kinase adapter is not represented in the public databases by a protein accession but is described in this patent by SEQ ID NO: 79. The nucleotide sequence of an unannotated EST corresponding to the novel PI-3-kinase adapter is represented in the public sequence databases by the accession R62339 and is described in this patent by SEQ ID NO: 80. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD and peripheral arterial disease. The novel PI-3-kinase adapter is preferentially induced by hypoxia in monocytes or macrophages, indicating utility of the encoded protein in the design of therapeutic, prognostic and diagnostic products addressing diseases involving macrophages and hypoxia. In a gene array analysis it is expressed in hypoxic monocytes and macrophages at levels 6-fold higher than the median expression level of this gene throughout 9 other cell types in either normoxia or hypoxia. In more sensitive TaqMan analysis the novel PI-3-kinase adapter it is found to be expressed at approximately 1000 times the levels of 9 other cell types, all exposed to hypoxia for 18 hr. The relevance of the novel PI-3-kinase adapter to human disease is also appreciated from comparison with a related murine gene, BCAP. It is known that this gene is phosphorylated by the tyrosine kinase, Syk. We also show novel data regarding Syk, in that it is also induced in response to hypoxia in a tissue specific manner identical to that of the novel PI-3-kinase adapter. Therefore the biological relevance and utility of our discovery of hypoxic induction of the novel PI-3-kinase adapter gene is further highlighted.

Clone p1F1 represents an unannotated EST. The protein sequence encoded by this EST is not represented in the public databases by a protein accession. The nucleotide sequence is represented in the public sequence databases by the accession AA489477 and is described in this patent by SEQ ID NO: 82. Hypoxia is an important feature of several diseases, and genes that respond to this stimulus are therefore implicated in the pathogenesis and have utility in the design of therapeutic, prognostic and diagnostic products.

Clone p1E7 represents a novel Metallothionein. The protein sequence encoded by Novel Metallothionein is not represented in the public databases by a protein accession but is described in this patent by SEQ ID NO: 83. The nucleotide sequence is represented in the public sequence databases by the accession R06601 and is described in this patent by SEQ ID NO: 84. Metallothioneins can act as an antioxidant and free-radical scavenger and are therefore protective against cell death in hypoxia. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors that mediate the response to hypoxia of several genes, and have them selves been implicated in specific diseases. By adenoviral over-expression of HIF1alpha we show augmentation of the hypoxic induction of certain genes, further confirming their status as responsive to hypoxia. The novel Metallothionein represented by SEQ ID NO: 84 has been shown to be induced by hypoxia to a greater degree following adenoviral over-expression of HIF1 alpha. Hepatocytes are the main cell type of the liver and genes that are induced in response to hypoxia in this cell type are relevant to development of diagnostics and therapeutics towards liver diseases involving hypoxia, including cirrhosis. The novel Metallothionein represented by SEQ ID NO: 84 is preferentially induced by hypoxia in hepatocytes. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The novel Metallothionein represented by SEQ ID NO: 84 is induced in macrophages activated by LPS and gamma interferon.

Clone p1E6 represents EGL nine (C. elegans) homolog 3. The protein sequence encoded by EGL nine (C. elegans) homolog 3 is represented in the public databases by the accession NP_{—}071356 and is described in this patent by SEQ ID NO: 85. The nucleotide sequence is represented in the public sequence databases by the accession NM_{—}022073 and is described in this patent by SEQ ID NO: 86. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors that mediate the response to hypoxia of several genes, and have them selves been implicated in specific diseases. By adenoviral over-expression of EPAS1 we show augmentation of the hypoxic induction of certain genes, further confirming their status as responsive to hypoxia. EGL nine (C. elegans) homolog 3 has been shown to be induced by hypoxia to a greater degree following adenoviral over-expression of EPAS1. Its preferential regulation by EPAS1 provides a route to preferential intervention, to avoid toxicity to other tissues. Hepatocytes are the main cell type of the liver and genes that are induced in response to hypoxia in this cell type are relevant to development of diagnostics and therapeutics towards liver diseases involving hypoxia, including cirrhosis. EGL nine (C. elegans) homolog 3 is preferentially induced by hypoxia in hepatocytes. We find that EGLN3 and a related human gene C1orf12 (seq ID 89/90) both of which are predicted to be proline hydroxylases, are expressed at differing absolute expression levels in different tissues. For instance, in the hypoxic hepatocyte (6 hr) the normalized expression values of EGLN and c1orf12 are 0.015 and 0.0074 respectively, i.e. EGLN being the dominant gene. In contrast, in the neuroblastoma cell line SH-SY5Y, the normalized expression values of EGLN and c1orf12 after 6 hr hypoxia are 0.0012 and 0.108 respectively, i.e. c1orf12 being the dominant gene by a large margin. This data demonstrates that c1ORF12 and EGLN3 are not constitutively expressed at an equal amount in different tissues indicating specificity of function. Therefore therapeutic products may be developed based on this data, with the goal of modulating proline hydroxylation of target proteins (such as HIF1alpha) in specific tissues, based on the differing expression profile of c1ORF12 and EGLN3 in those tissues.

Clone p1D14 represents C1orf12. The protein sequence encoded by C1orf12 is represented in the public databases by the accession NP_{—}071334 and is described in this patent by SEQ ID NO: 89. The nucleotide sequence is represented in the public sequence databases by the accession NM_{—}022051 and is described in this patent by SEQ ID NO: 90. Hypoxia is an important feature of several diseases, and genes that respond to this stimulus are therefore implicated in the pathogenesis and have utility in the design of therapeutic, prognostic and diagnostic products. We find that C1orf12 and a related human gene EGLN3 (seq ID 85/86) both of which are predicted to be proline hydroxylases, are expressed at differing absolute expression levels in different tissues. For instance, in the hypoxic hepatocyte (6 hr) the normalized expression values of EGLN and c1orf12 are 0.015 and 0.0074 respectively, i.e. EGLN being the dominant gene. In contrast, in the neuroblastoma cell line SH-SY5Y, the normalized expression values of EGLN and c1orf12 after 6 hr hypoxia are 0.0012 and 0.108 respectively, i.e. c1orf12 being the dominant gene by a large margin. This data demonstrates that c1ORF12 and EGLN3 are not constitutively expressed at an equal amount in different tissues indicating specificity of function. Therefore therapeutic products may be developed based on this data, with the goal of modulating proline hydroxylation of target proteins (such as HIF1alpha) in specific tissues, based on the differing expression profile of c1ORF12 and EGLN3 in those tissues.

Clone p2B1 represents PRAME. The protein sequence encoded by PRAME is represented in the public databases by the accession NP_{—}006106 and is described in this patent by SEQ ID NO: 87. The nucleotide sequence is represented in the public sequence databases by the accession NM_{—}006115 and is described in this patent by SEQ ID NO: 88. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, PRAME is up-regulated in the malignant tissue as compared to adjacent normal tissue in at least one patient. PRAME is a well-known tumour-associated antigen. Our surprising demonstration of its hypoxia-regulation provides for an important diagnostic test to distinguish false-positive results. In addition, we show the relevance of PRAME to hypoxia-related functions of tumours such as angiogenesis.

Clones p1D17 and p1P14 represent Semaphorin 4b. The protein sequence encoded by Semaphorin 4b is represented in the public databases by the accession BAB21836 and is described in this patent by SEQ ID NO: 91. The nucleotide sequence is represented in the public sequence databases by the accession AB051532 and is described in this patent by SEQ ID NO: 92. Hypoxia is an important feature of several diseases, and genes that respond to this stimulus are therefore implicated in the pathogenesis and have utility in the design of therapeutic, prognostic and diagnostic products. The response of renal epithelial cells to hypoxia is pertinent to kidney failure, especially regarding the medullary tissue. Semaphorin 4b is preferentially induced by hypoxia in renal epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Semaphorin 4b is induced in macrophages activated by LPS and gamma interferon.. Semaphorin 4b is also induced by the the presence of reactive oxygen species. We expect it to have a pro-inflammatory role, and its inhibition may have an anti-inflammatory effect. We have cited elsewhere in this specification that a plexin is hypoxia-regulated, and we propose a functional relationship between these two molecules. Hypoxia is frequently found in human tumours where macrophage infiltrates are also found. In a series of 5 patients with either ovarian or breast cancer, Semaphorin 4b is up-regulated in the malignant tissue as compared to adjacent normal tissue in at least one patient. Semaphorin 4b is also induced in response to skuperoxide radicals, as found in various disease states, implying utility. Semaphorin 4b is predicted to function in modulating several cellular processes key to human disease, including angiogenesis, inflammation, immune cell migration and tissue remodelling. Other Semaphorins including Semaphorin E, which are induced in response to hypoxia will also be implicated in these disease processes and have utility as described for Semaphorin 4b.

Clone p1C24 represents SLC25A19. The protein sequence encoded by SLC25A19 is represented in the public databases by the accession NP_{—}068380 and is described in this patent by SEQ ID NO: 93. The nucleotide sequence is represented in the public sequence databases by the accession NM_{—}021734 and is described in this patent by SEQ ID NO: 94. SLC25A19 transports deoxynucleotides into mitochondria and is therefore essential for mtDNA synthesis. Hypoxia is an important feature of several diseases, and genes that respond to this stimulus are therefore implicated in the pathogenesis and have utility in the design of therapeutic, prognostic and diagnostic products.

Clone p1D3 represents Serine carboxypeptidase 1. The protein sequence encoded by Serine carboxypeptidase 1 is represented in the public databases by the accession NP_{—}067639 and is described in this patent by SEQ ID NO: 95. The nucleotide sequence is represented in the public sequence databases by the accession NM_{—}021626 and is described in this patent by SEQ ID NO: 96. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Serine carboxypeptidase 1 is repressed in macrophages activated by LPS and gamnma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Serine carboxypeptidase 1 is induced in macrophages activated by TNFalpha. Increased serine carboxypeptidase activity in glial cells has been shown to result in neurological abnormalities, due to the degradation of essential neuro-active factors. Similarly, peripheral neurological disease could result from such activity in macrophages. Our demonstration of hypoxia regulation of serine carboxypeptidase activity opens a route for diagnosis and treatment of these diseases. Hypoxia is frequently found in human tumours where macrophage infiltrates are also found. In a series of 5 patients with either ovarian or breast cancer, Serine carboxypeptidase 1 is down-regulated in the malignant tissue as compared to adjacent normal tissue in at least one patient.

Clone p1E14 represents an unknown mRNA (schizophrenia-linked). The protein sequence encoded by the unknown mRNA (schizophrenia-linked) is not represented in the public databases by a protein accession. The nucleotide sequence is represented in the public sequence databases by the accession AY010112 and is described in this patent by SEQ ID NO: 98. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Unknown mRNA (schizophrenia-linked) is induced in macrophages activated by TNFalpha. There are many enzymic activities that can give rise to neurological abnormalities, and their hypoxia regulation is pertinent to the diagnosis and treatment of such diseases, including schizophrenia.

Clone p1E20 represents Myo-inositol monophosphatase A3. The protein sequence encoded by Myo-inositol monophosphatase A3 is represented in the public databases by the accession AAK52336 and is described in this patent by SEQ ID NO: 99. The nucleotide sequence is represented in the public sequence databases by the accession NM_{—}017813 and is described in this patent by SEQ ID NO: 100. Hypoxia is an important feature of several diseases, and genes that respond to this stimulus are therefore implicated in the pathogenesis and have utility in the design of therapeutic, prognostic and diagnostic products. As refered to elsewhere in this specification, we have found several components of the phosphatidylinisotol second messenger system to be hypoxia-regulated. This system has profound effects which are relevant to many diseases with known associations with hypoxia and ischaemia. Local and transient ischaemia is relevant to such diseases as rheumatoid arthritis and atherosclerosis, and also potentially to such diseases as schizophrenia and bi-polar disorder. It is instructive that lithium, which is a well-recognised treatment for affective disorders, appears to operate via the phosphatidylinisotol system [Pettegrew et al 2001, Bipolar Disord 3:189-201]. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Myo-inositol monophosphatase A3 is repressed in macrophages activated by LPS and gamma interferon.

Clone p2A24 represents an unannotated EST. The protein sequence encoded by this EST is not represented in the public databases by a protein accession. The nucleotide sequence is represented in the public sequence databases by the accession AA521314 and is described in this patent by SEQ ID NO: 102. Hypoxia is an important feature of several diseases, and genes that respond to this stimulus are therefore implicated in the pathogenesis and have utility in the design of therapeutic, prognostic and diagnostic products.

Clone p1E17 represents hypothetical protein FLJ31668. The protein sequence encoded by hypothetical protein FLJ31668 is represented in the public databases by the accession BAB71124 and is described in this patent by SEQ ID NO: 103. The nucleotide sequence is represented in the public sequence databases by the accession AK056230 and is described in this patent by SEQ ID NO: 104. Hypoxia is an important feature of several diseases, and genes that respond to this stimulus are therefore implicated in the pathogenesis and have utility in the design of therapeutic, prognostic and diagnostic products.

Clone p1E19 represents an unannotated EST. The protein sequence encoded by this EST is not represented in the public databases by a protein accession. The nucleotide sequence is represented in the public sequence databases by the accession R51835 and is described in this patent by SEQ ID NO: 106. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the EST represented by SEQ ID NO: 106 is up-regulated in the malignant tissue as compared to adjacent normal tissue in at least one patient.

Clone p1E15 represents cDNA YI27F12. The protein sequence encoded by cDNA YI27F12 is not represented in the public databases by a protein accession. The nucleotide sequence is represented in the public sequence databases by the accession AF075018 and is described in this patent by SEQ ID NO: 108. Hypoxia is an important feature of several diseases, and genes that respond to this stimulus are therefore implicated in the pathogenesis and have utility in the design of therapeutic, prognostic and diagnostic products. The cDNA YI27F12 is induced in macrophages treated with the inhibitory cytokine IL-10. The cDNA YI27F12 is repressed in macrophages activated by IL-17. We expect the product of cDNA YI27F12 to have an anti-inflammatory role.

Clone p1E11 represents an unannotated EST. The protein sequence encoded by this EST is not represented in the public databases by a protein accession. The nucleotide sequence is represented in the public sequence databases by the accession R69248 and is described in this patent by SEQ ID NO: 110. Hypoxia is an important feature of several diseases, and genes that respond to this stimulus are therefore implicated in the pathogenesis and have utility in the design of therapeutic, prognostic and diagnostic products.

Clone p1E23 represents cDNA FLJ14041 fis, clone HEMBA1005780. The protein sequence encoded by cDNA FLJ14041 fis, clone HEMBA1005780 is not represented in the public databases by a protein accession. The nucleotide sequence is represented in the public sequence databases by the accession AK024103 and is described in this patent by SEQ ID NO: 112. Hypoxia is an important feature of several diseases, and genes that respond to this stimulus are therefore implicated in the pathogenesis and have utility in the design of therapeutic, prognostic and diagnostic products.

Clone p1E21 represents Glutamate-cysteine ligase, modifier subunit. The protein sequence encoded by Glutamate-cysteine ligase, modifier subunit is represented in the public databases by the accession NP_{—}002052 and is described in this patent by SEQ ID NO: 113. The nucleotide sequence is represented in the public sequence databases by the accession NM_{—}002061 and is described in this patent by SEQ ID NO: 114. Hypoxia is an important feature of several diseases, and genes that respond to this stimulus are therefore implicated in the pathogenesis and have utility in the design of therapeutic, prognostic and diagnostic products. Glutamate-cysteine ligase is the rate-limiting enzyme of glutathione synthesis, and this enzyme is relevant to cell survival under stress. Hypoxia is frequently found in human tumours where macrophage infiltrates are also found. In a series of 5 patients with either ovarian or breast cancer, Glutamate-cysteine ligase, modifier subunit is up-regulated in the malignant tissue as compared to adjacent normal tissue in at least one patient.

Clone p1D23 represents PTEN. The protein sequence encoded by PTEN is represented in the public databases by the accession NP_{—}000305 and is described in this patent by SEQ ID NO: 115. The nucleotide sequence is represented in the public sequence databases by the accession NM_{—}000314 and is described in this patent by SEQ ID NO: 116. PTEN is a member of the mixed function, serine/threonine/tyrosine phosphatase subfamily of protein phosphatases. Its physiological substrates, however, are primarily 3-phosphorylated inositol phospholipids, which are products of phosphoinositide 3-kinases [Downes et al 2001, Biochem Soc Trans 29:846-51]. Hypoxia-regulation of this gene is a further element in the hypoxic regulation of this important second messenger system. Hypoxia is an important feature of several diseases, and genes that respond to this stimulus are therefore implicated in the pathogenesis and have utility in the design of therapeutic, prognostic and diagnostic products.

Clone p1D24 represents an unannotated EST. The protein sequence encoded by this EST is not represented in the public databases by a protein accession. The nucleotide sequence is represented in the public sequence databases by the accession T73780 and is described in this patent by SEQ ID NO: 118. Hypoxia is an important feature of several diseases, and genes that respond to this stimulus are therefore implicated in the pathogenesis and have utility in the design of therapeutic, prognostic and diagnostic products. The response of renal epithelial cells to hypoxia is pertinent to kidney failure, especially regarding the medullary tissue. The EST represented by SEQ ID NO: 118 is preferentially induced by hypoxia in renal epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The EST represented by SEQ ID NO: 118 is induced in macrophages activated by LPS and gamma interferon.

Clones p1D22 and p1G5 represent MAX-interacting protein 1. The protein sequence encoded by MAX-interacting protein 1 is represented in the public databases by the accession NP_{—}005953 and is described in this patent by SEQ ID NO: 119 and 279. The nucleotide sequence is represented in the public sequence databases by the accession NM_{—}005962 and is described in this patent by SEQ ID NO: 120 and 280. MAX-interacting protein 1 is a negative regulator of myc oncoprotein with tumor suppressor properties. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. MAX-interacting protein 1 is repressed in macrophages activated by LPS and gamma interferon.

Clone p1E2 represents Mannosidase, alpha, class 1A, member 1. The protein sequence encoded by Mannosidase, alpha, class 1A, member 1 is represented in the public databases by the accession NP_{—}005898 and is described in this patent by SEQ ID NO: 121. The nucleotide sequence is represented in the public sequence databases by the accession NM_{—}005907 and is described in this patent by SEQ ID NO: 122. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, Mannosidase, alpha, class 1A, member 1 is down-regulated in the malignant tissue as compared to adjacent normal tissue in at least one patient.

Clone p1E1 represents an unannotated EST. The protein sequence encoded by this EST is not represented in the public databases by a protein accession. The nucleotide sequence is represented in the public sequence databases by the accession AA446361 and is described in this patent by SEQ ID NO: 124. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The EST represented by SEQ ID NO: 124 is repressed in macrophages activated by LPS and gamma interferon.

Clone p1E4 represents an unannotated EST. The protein sequence encoded by this EST is not represented in the public databases by a protein accession. The nucleotide sequence is represented in the public sequence databases by the accession AA931411 and is described in this patent by SEQ ID NO: 126. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The EST represented by SEQ ID NO: 126 is repressed in macrophages activated by LPS and gamma interferon. We expect this gene product to have an anti-inflammatory role. Hypoxia is frequently found in human tumours where macrophage infiltrates are also found. In a series of 5 patients with either ovarian or breast cancer, the EST represented by SEQ ID NO: 126 is down-regulated in the malignant tissue as compared to adjacent normal tissue in at least one patient.

Clone p1D18 represents cDNA FLJ13443 fis, clone PLACE1002853. The protein sequence encoded by cDNA FLJ13443 fis, clone PLACE1002853 is not represented in the public databases by a protein accession. The nucleotide sequence is represented in the public sequence databases by the accession AK023505 and is described in this patent by SEQ ID NO: 128. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The cDNA FLJ13443 fis, clone PLACE1002853 is repressed in macrophages activated by LPS and gamma interferon. We expect it to have an anti-inflammatory role.

Clone p1D21 represents hypothetical protein FLJ22622. The protein sequence encoded by hypothetical protein FLJ22622 is represented in the public databases by the accession BAB15424 and is described in this patent by SEQ ID NO: 129. The nucleotide sequence is represented in the public sequence databases by the accession NM_{—}025151 and is described in this patent by SEQ ID NO: 130. Hypoxia is an important feature of several diseases, and genes that respond to this stimulus are therefore implicated in the pathogenesis and have utility in the design of therapeutic, prognostic and diagnostic products. The response of renal epithelial cells to hypoxia is pertinent to kidney failure, especially regarding the medullary tissue. Hypothetical protein FLJ22622 is preferentially induced by hypoxia in renal epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein FLJ22622 is repressed in macrophages activated by LPS and gamma interferon. We expect it to have an anti-inflammatory role. Hypoxia is frequently found in human tumours where macrophage infiltrates are also found. In a series of 5 patients with either ovarian or breast cancer, hypothetical protein FLJ22622 is up-regulated in the malignant tissue as compared to adjacent normal tissue in at least one patient.

Clone p1C22 represents CD84-H1. The protein sequence encoded by CD84-H1 is represented in the public databases by the accession AAK69052 and is described in this patent by SEQ ID NO: 131. The nucleotide sequence is represented in the public sequence databases by the accession AF275725 and is described in this patent by SEQ ID NO: 132. Hypoxia is an important feature of several diseases, and genes that respond to this stimulus are therefore implicated in the pathogenesis and have utility in the design of therapeutic, prognostic and diagnostic products.

Clone p1C23 represents hypothetical protein FLJ12832. The protein sequence encoded by hypothetical protein FLJ12832 is represented in the public databases by the accession XP_{—}043394 and is described in this patent by SEQ ID NO: 133. The nucleotide sequence is represented in the public sequence databases by the accession AK022894 and is described in this patent by SEQ ID NO: 134. Hypothetical protien FLJ12832 is a putative ubiquitin as it shows high structural similarity to ubiquitin C and contains a ubiquitin domain. Hypoxia is an important feature of several diseases, and genes that respond to this stimulus are therefore implicated in the pathogenesis and have utility in the design of therapeutic, prognostic and diagnostic products.

Clone p1D11 represents an unannotated EST. The protein sequence encoded by this EST is not represented in the public databases by a protein accession. The nucleotide sequence is represented in the public sequence databases by the accession AA251748 and is described in this patent by SEQ ID NO: 136. Hypoxia is an important feature of several diseases, and genes that respond to this stimulus are therefore implicated in the pathogenesis and have utility in the design of therapeutic, prognostic and diagnostic products.

Clones p1E3 and p1F16 represent CYP1B1. The protein sequence encoded by CYP1B1 is represented in the public databases by the accession NP_{—}000095 and is described in this patent by SEQ ID NO: 137 and 325. The nucleotide sequence is represented in the public sequence databases by the accession NM_{—}000104 and is described in this patent by SEQ ID NO: 138 and 326. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. CYP1B1 is preferentially induced by hypoxia in monocytes or macrophages and a restricted number of other cell types. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. CYP1B1 is repressed in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. CYP1B1 is induced in macrophages activated by TNFalpha. Hypoxia is frequently found in human tumours where macrophage infiltrates are also found. In a series of 5 patients with either ovarian or breast cancer, CYP1B1 is up-regulated and also down regulated in the malignant tissue as compared to adjacent normal tissue in at least one patient.

Clone p1D20 represents hypothetical protein KIAA1125. The protein sequence encoded by hypothetical protein KIAA1125 is represented in the public databases by the accession XP_{—}012932 and is described in this patent by SEQ ID NO: 139. The nucleotide sequence is represented in the public sequence databases by the accession AB032951 and is described in this patent by SEQ ID NO: 140. Hypoxia is an important feature of several diseases, and genes that respond to this stimulus are therefore implicated in the pathogenesis and have utility in the design of therapeutic, prognostic and diagnostic products.

Clone p1E5 represents Hepcidin antimicrobial peptide. The protein sequence encoded by Hepcidin antimicrobial peptide is represented in the public databases by the accession NP_{—}066998 and is described in this patent by SEQ ID NO: 141. The nucleotide sequence is represented in the public sequence databases by the accession NM_{—}021175 and is described in this patent by SEQ ID NO: 142. Hypoxia is an important feature of several diseases, and genes that respond to this stimulus are therefore implicated in the pathogenesis and have utility in the design of therapeutic, prognostic and diagnostic products. Hepatocytes are the main cell type of the liver and genes that are induced in response to hypoxia in this cell type are relevant to development of diagnostics and therapeutics towards liver diseases involving hypoxia, including cirrhosis. Hepcidin antimicrobial peptide is preferentially induced by hypoxia in hepatocytes. Hepcidin antimicrobial peptide is induced in macrophages treated with the inhibitory cytokine IL-10. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Hepcidin antimicrobial peptide is repressed in macrophages activated by TNFalpha.

Clone p1D19 represents an unannotated EST. The protein sequence encoded by this EST is not represented in the public databases by a protein accession. The nucleotide sequence is represented in the public sequence databases by the accession R68736 and is described in this patent by SEQ ID NO: 144. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The EST represented by SEQ ID NO: 144 is induced in macrophages activated by LPS and gamma interferon and is also induced in macrophages activated by IL-15. We expect the gene product relevant to the EST represented by SEQ ID NO: 144 to have a pro-inflammatory role, and its inhibition may have an anti-inflammatory effect.

Clone p2A15 represents Sialyltransferase. The protein sequence encoded by Sialyltransferase is represented in the public databases by the accession NP_{—}006447 and is described in this patent by SEQ ID NO: 145. The nucleotide sequence is represented in the public sequence databases by the accession NM_{—}006456 and is described in this patent by SEQ ID NO: 146. Hypoxia is an important feature of several diseases, and genes that respond to this stimulus are therefore implicated in the pathogenesis and have utility in the design of therapeutic, prognostic and diagnostic products.

Clone p1I14 represents cDNA DKFZp564D016. The protein sequence encoded by cDNA DKFZp564D016 is not represented in the public databases by a protein accession. The nucleotide sequence is represented in the public sequence databases by the accession AL050021 and is described in this patent by SEQ ID NO: 148. Hypoxia is an important feature of several diseases, and genes that respond to this stimulus are therefore implicated in the pathogenesis and have utility in the design of therapeutic, prognostic and diagnostic products.

Clone p1I2 represents cDNA FLJ11302 fis, clone PLACE1009971. The protein sequence encoded by cDNA FLJ11302 fis, clone PLACE1009971 is not represented in the public databases by a protein accession. The nucleotide sequence is represented in the public sequence databases by the accession AK002164 and is described in this patent by SEQ ID NO: 150. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The cDNA FLJ11302 fis, clone PLACE1009971 is repressed in macrophages activated by LPS and gamma interferon. We expect it to have an anti-inflammatory role.

Clone p1I12 represents hypothetical protein MGC4549. The protein sequence encoded by hypothetical protein MGC4549 is represented in the public databases by the accession XP_{—}032794 and is described in this patent by SEQ ID NO: 151. The nucleotide sequence is represented in the public sequence databases by the accession NM_{—}032377 and is described in this patent by SEQ ID NO: 152. Hypoxia is an important feature of several diseases, and genes that respond to this stimulus are therefore implicated in the pathogenesis and have utility in the design of therapeutic, prognostic and diagnostic products. Hypothetical protein MGC4549 is induced in macrophages treated with the inhibitory cytokine IL-10. Hypothetical protein MGC4549 is repressed in macrophages activated by IL-17 and is also repressed in macrophages activated by IL-15. We expect it to have an anti-inflammatory role.

Clone p1I3 represents ELMO2. The protein sequence encoded by ELMO2 is represented in the public databases by the accession AAL14467 and is described in this patent by SEQ ID NO: 153. The nucleotide sequence is represented in the public sequence databases by the accession XM_{—}012933 and is described in this patent by SEQ ID NO: 154. Hypoxia is an important feature of several diseases, and genes that respond to this stimulus are therefore implicated in the pathogenesis and have utility in the design of therapeutic, prognostic and diagnostic products. This gene has been shown recently to promote phagocytosis and cell shape changes [Gumienny et al 2001, Cell 107:27-41]. These functions are typical of the macrophage, and are likely to play a role in macrophage-associated diseases.

Clone p1I10 represents an unannotated EST. The protein sequence encoded by this EST is not represented in the public databases by a protein accession. The nucleotide sequence is represented in the public sequence databases by the accession AA420992 and is described in this patent by SEQ ID NO: 156. Hypoxia is an important feature of several diseases, and genes that respond to this stimulus are therefore implicated in the pathogenesis and have utility in the design of therapeutic, prognostic and diagnostic products.

Clone p1H18 represents Ubiquitin specific protease 7. The protein sequence encoded by Ubiquitin specific protease 7 is represented in the public databases by the accession NP_{—}003461 and is described in this patent by SEQ ID NO: 157. The nucleotide sequence is represented in the public sequence databases by the accession NM_{—}003470 and is described in this patent by SEQ ID NO: 158. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, Ubiquitin specific protease 7 is up-regulated in the malignant tissue as compared to adjacent normal tissue in at least one patient. We expect decreased activity of the gene product to have an anti-tumour effect.

Clone p1H24 represents Nucleolar phosphoprotein Nopp34. The protein sequence encoded by Nucleolar phosphoprotein Nopp34 is represented in the public databases by the accession NP_{—}115766 and is described in this patent by SEQ ID NO: 159. The nucleotide sequence is represented in the public sequence databases by the accession NM_{—}032390 and is described in this patent by SEQ ID NO: 160. Hypoxia is an important feature of several diseases, and genes that respond to this stimulus are therefore implicated in the pathogenesis and have utility in the design of therapeutic, prognostic and diagnostic products.

Clone p1E22 represents cDNA FLJ13618 fis, clone PLACE1010925. The protein sequence encoded by cDNA FLJ13618 fis, clone PLACE1010925 is not represented in the public databases by a protein accession. The nucleotide sequence is represented in the public sequence databases by the accession AK023680 and is described in this patent by SEQ ID NO: 162. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The cDNA FLJ13618 fis, clone PLACE1010925 is induced in macrophages activated by LPS and gamma interferon.

Clone p1H21 represents hypothetical protein FLJ13511. The protein sequence encoded by hypothetical protein FLJ13511 is represented in the public databases by the accession NP_{—}149014 and is described in this patent by SEQ ID NO: 163. The nucleotide sequence is represented in the public sequence databases by the accession NM_{—}033025 and is described in this patent by SEQ ID NO: 164. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Hypothetical protein FLJ13511 is preferentially induced by hypoxia in monocytes or macrophages and a restricted number of other cell types. Hypoxia is frequently found in human tumours where macrophage infiltrates are also found. In a series of 5 patients with either ovarian or breast cancer, hypothetical protein FLJ13511 is down-regulated in the malignant tissue as compared to adjacent normal tissue in at least one patient.

Clone p1I1 represents Ribosomal RNA intergenic spacer. The protein sequence encoded by Ribosomal RNA intergenic spacer is not represented in the public databases by a protein accession. The nucleotide sequence is represented in the public sequence databases by the accession AA664228 and is described in this patent by SEQ ID NO: 166. Hypoxia is an important feature of several diseases, and genes that respond to this stimulus are therefore implicated in the pathogenesis and have utility in the design of therapeutic, prognostic and diagnostic products.

Clone p1H14 represents an unannotated EST. The protein sequence encoded by this EST is not represented in the public databases by a protein accession. The nucleotide sequence is represented in the public sequence databases by the accession R44397 and is described in this patent by SEQ ID NO: 168. Hypoxia is an important feature of several diseases, and genes that respond to this stimulus are therefore implicated in the pathogenesis and have utility in the design of therapeutic, prognostic and diagnostic products.

Clone p1H11 represents Carboxypeptidase M. The protein sequence encoded by Carboxypeptidase M is represented in the public databases by the accession NP_{—}001865 and is described in this patent by SEQ ID NO: 169. The nucleotide sequence is represented in the public sequence databases by the accession NM_{—}001874 and is described in this patent by SEQ ID NO: 170. Hypoxia is an important feature of several diseases, and genes that respond to this stimulus are therefore implicated in the pathogenesis and have utility in the design of therapeutic, prognostic and diagnostic products.

Clone p1H17 represents an unannotated EST. The protein sequence encoded by this EST is not represented in the public databases by a protein accession. The nucleotide sequence is represented in the public sequence databases by the accession W87747 and is described in this patent by SEQ ID NO: 172. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the EST represented by SEQ ID NO: 172 is up-regulated and also down-regulated in the malignant tissue as compared to adjacent normal tissue in at least one patient.

Clone p1H12 represents an unannotated EST. The protein sequence encoded by this EST is not represented in the public databases by a protein accession. The nucleotide sequence is represented in the public sequence databases by the accession AA973568 and is described in this patent by SEQ ID NO: 174. Hypoxia is an important feature of several diseases, and genes that respond to this stimulus are therefore implicated in the pathogenesis and have utility in the design of therapeutic, prognostic and diagnostic products.

Clone p1H7 represents an unannotated EST. The protein sequence encoded by this EST is not represented in the public databases by a protein accession. The nucleotide sequence is represented in the public sequence databases by the accession T98529 and is described in this patent by SEQ ID NO: 176. Hypoxia is an important feature of several diseases, and genes that respond to this stimulus are therefore implicated in the pathogenesis and have utility in the design of therapeutic, prognostic and diagnostic products.

Clone p1H15 represents an unannotated EST. The protein sequence encoded by this EST is not represented in the public databases by a protein accession. The nucleotide sequence is represented in the public sequence databases by the accession AA022679 and is described in this patent by SEQ ID NO: 178. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the EST represented by SEQ ID NO: 178 is up-regulated and also down-regulated in the malignant tissue as compared to adjacent normal tissue in at least one patient.

Clone p1H20 represents an unannotated EST. The protein sequence encoded by EST is not represented in the public databases by a protein accession. The nucleotide sequence is represented in the public sequence databases by the accession H17921 and is described in this patent by SEQ ID NO: 180. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the EST represented by SEQ ID NO: 180 is up-regulated in the malignant tissue as compared to adjacent normal tissue in at least one patient. We expect decreased activity of the gene product to have an anti-tumour effect.

Clone p1H8 represents ABL. The protein sequence encoded by ABL is represented in the public databases by the accession NP_{—}009297 and is described in this patent by SEQ ID NO: 181. The nucleotide sequence is represented in the public sequence databases by the accession NM_{—}007313 and is described in this patent by SEQ ID NO: 182. Hypoxia is an important feature of several diseases, and genes that respond to this stimulus are therefore implicated in the pathogenesis and have utility in the design of therapeutic, prognostic and diagnostic products. ABL is induced in macrophages treated with the inhibitory cytokine IL-10. ABL is repressed in macrophages activated by IL-17 and is also repressed in macrophages activated by IL-15. We expect it to have an anti-inflammatory role Hypoxia is frequently found in human tumours where macrophage infiltrates are also found. In a series of 5 patients with either ovarian or breast cancer, ABL is up-regulated in the malignant tissue as compared to adjacent normal tissue in at least one patient.

Clone p1H16 represents an unannotated EST. The protein sequence encoded by this EST is not represented in the public databases by a protein accession. The nucleotide sequence is represented in the public sequence databases by the accession W91958 and is described in this patent by SEQ ID NO: 184. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the EST represented by SEQ ID NO: 184 is up-regulated in the malignant tissue as compared to adjacent normal tissue in at least one patient.

Clone p1H9 represents an unannotated EST. The protein sequence encoded by this EST is not represented in the public databases by a protein accession. The nucleotide sequence is represented in the public sequence databases by the accession R63694 and is described in this patent by SEQ ID NO: 186. Hypoxia is an important feature of several diseases, and genes that respond to this stimulus are therefore implicated in the pathogenesis and have utility in the design of therapeutic, prognostic and diagnostic products.

Clone p1H23 represents hypothetical protein FLJ21094. The protein sequence encoded by hypothetical protein FLJ21094 is represented in the public databases by the accession AAH14003 and is described in this patent by SEQ ID NO: 187. The nucleotide sequence is represented in the public sequence databases by the accession AK024747 and is described in this patent by SEQ ID NO: 188. Hypoxia is an important feature of several diseases, and genes that respond to this stimulus are therefore implicated in the pathogenesis and have utility in the design of therapeutic, prognostic and diagnostic products.

Clone p1H10 represents an unannotated EST. The protein sequence encoded by this EST is not represented in the public databases by a protein accession. The nucleotide sequence is represented in the public sequence databases by the accession AA909912 and is described in this patent by SEQ ID NO: 190. Hypoxia is an important feature of several diseases, and genes that respond to this stimulus are therefore implicated in the pathogenesis and have utility in the design of therapeutic, prognostic and diagnostic products.

Clone p1H6 represents an unannotated EST. The protein sequence encoded by this EST is not represented in the public databases by a protein accession. The nucleotide sequence is represented in the public sequence databases by the accession T99032 and is described in this patent by SEQ ID NO: 192. Hypoxia is an important feature of several diseases, and genes that respond to this stimulus are therefore implicated in the pathogenesis and have utility in the design of therapeutic, prognostic and diagnostic products. The EST represented by SEQ ID NO: 192 is induced in macrophages treated with the inhibitory cytokine IL-10. The EST represented by SEQ ID NO: 192 is repressed in macrophages activated by IL-15. We expect it to have an anti-inflammatory role.

Clone p1H13 represents an unannotated EST. The protein sequence encoded by this EST is not represented in the public databases by a protein accession. The nucleotide sequence is represented in the public sequence databases by the accession H52503 and is described in this patent by SEQ ID NO: 194. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The EST represented by SEQ ID NO: 194 is repressed in macrophages activated by LPS and gamma interferon.

Clone p1H19 represents an unannotated EST. The protein sequence encoded by this EST is not represented in the public databases by a protein accession. The nucleotide sequence is represented in the public sequence databases by the accession AA127017 and is described in this patent by SEQ ID NO: 196. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the EST represented by the SEQ ID NO: 196 is down-regulated in the malignant tissue as compared to adjacent normal tissue in at least one patient.

Clone p1G22 represents an unannotated EST. The protein sequence encoded by this EST is not represented in the public databases by a protein accession. The nucleotide sequence is represented in the public sequence databases by the accession R38647 and is described in this patent by SEQ ID NO: 198. Hypoxia is an important feature of several diseases, and genes that respond to this stimulus are therefore implicated in the pathogenesis and have utility in the design of therapeutic, prognostic and diagnostic products. Endothelial cells are key to angiogenesis, a process implicated in several diseases associated with hypoxia, including cancer and rheumatoid arthritis. The EST represented by SEQ ID NO: 198 is preferentially induced by hypoxia in endothelial cells. We expect this gene product to have a pro-angiogenic effect, and its inhibition to have an anti-angiogenic effect.

Clone p1G21 represents an unannotated EST. The protein sequence encoded by this EST is not represented in the public databases by a protein accession. The nucleotide sequence is represented in the public sequence databases by the accession T87233 and is described in this patent by SEQ ID NO: 200. Hypoxia is an important feature of several diseases, and genes that respond to this stimulus are therefore implicated in the pathogenesis and have utility in the design of therapeutic, prognostic and diagnostic products.

Clone p1H1 represents hypothetical protein FLJ10826. The protein sequence encoded by hypothetical protein FLJ10826 is represented in the public databases by the accession BAB14226 and is described in this patent by SEQ ID NO: 201. The nucleotide sequence is represented in the public sequence databases by the accession NM_{—}018233 and is described in this patent by SEQ ID NO: 202. Hypoxia is an important feature of several diseases, and genes that respond to this stimulus are therefore implicated in the pathogenesis and have utility in the design of therapeutic, prognostic and diagnostic products.

Clone p1G20 represents cDNA YO23H03. The protein sequence encoded by cDNA YO23H03 is not represented in the public databases by a protein accession. The nucleotide sequence is represented in the public sequence databases by the accession AF075053 and is described in this patent by SEQ ID NO: 204. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The cDNA YO23H03 is repressed in macrophages activated by LPS and gamma interferon.

Clone p1H5 represents hypothetical protein FLJ22690. The protein sequence encoded by hypothetical protein FLJ22690 is represented in the public databases by the accession NP_{—}078987 and is described in this patent by SEQ ID NO: 205. The nucleotide sequence is represented in the public sequence databases by the accession NM_{—}024711 and is described in this patent by SEQ ID NO: 206. Hypoxia is an important feature of several diseases, and genes that respond to this stimulus are therefore implicated in the pathogenesis and have utility in,the design of therapeutic, prognostic and diagnostic products. Endothelial cells are key to angiogenesis, a process implicated in several diseases associated with hypoxia, including cancer and rheumatoid arthritis. Hypothetical protein FLJ22690 is preferentially induced by hypoxia in endothelial cells. We expect this gene product to have a pro-angiogenic effect, and its inhibition to have an anti-angiogenic effect. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein FLJ22690 is induced in macrophages activated by IL-15.

Clone p1G19 represents Mitochondrion sequence. The protein sequence encoded by Mitochondrion sequence is represented in the public databases by the accession AAH05845 and is described in this patent by SEQ ID NO: 207. The nucleotide sequence is represented in the public sequence databases by the accession BC005845 and is described in this patent by SEQ ID NO: 208. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the Mitochondrion sequence represented by SEQ ID NO: 208 is down-regulated in the malignant tissue as compared to adjacent normal tissue in at least one patient.

Clone p1H2 represents Fatty acid binding protein 5. The protein sequence encoded by Fatty acid binding protein 5 is represented in the public databases by the accession NP_{—}001435 and is described in this patent by SEQ ID NO: 209. The nucleotide sequence is represented in the public sequence databases by the accession NM_{—}001444 and is described in this patent by SEQ ID NO: 210. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Fatty acid binding protein 5 is preferentially induced by hypoxia in monocytes or macrophages. Crucially and very recently, Fatty acid binding protein 5 expressed in macrophages has been shown to play a very important role in the development of atherosclerotic plaques [Layne et al 2001, FASEB J 15:2733-5]. Our demonstration of hypoxic-regulation of this gene not only makes clear how this gene can participate in disease initiation and progression, but provides for a potential route to diagnosis and therapy of atherosclerosis. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Fatty acid binding protein 5 is repressed in macrophages activated by TNFalpha.

Clone p1G18 represents Mitochondrion sequence. The protein sequence encoded by Mitochondrion sequence is not represented in the public databases by a protein accession. The nucleotide sequence is represented in the public sequence databases by the accession BC001612 and is described in this patent by SEQ ID NO: 212. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The Mitochondrion sequence represented by SEQ ID NO: 212 is repressed in macrophages activated by LPS and gamma interferon.

Clone p1H4 represents an unannotated EST. The protein sequence encoded by this EST is not represented in the public databases by a protein accession. The nucleotide sequence is represented in the public sequence databases by the accession AA679939 and is described in this patent by SEQ ID NO: 214. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The EST represented by SEQ ID NO: 214 is repressed in macrophages activated by IL-17. We expect it to have an anti-inflammatory role. Hypoxia is frequently found in human tumours where macrophage infiltrates are also found. In a series of 5 patients with either ovarian or breast cancer, the EST represented by the SEQ ID NO: 214 is down-regulated in the malignant tissue as compared to adjacent normal tissue in at least one patient.

Clone p1H3 represents an unannotated EST. The protein sequence encoded by this EST is not represented in the public databases by a protein accession. The nucleotide sequence is represented in the public sequence databases by the accession AA630167 and is described in this patent by SEQ ID NO: 216. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, the EST represented by SEQ ID NO: 216 is up-regulated and also down-regulated in the malignant tissue as compared to adjacent normal tissue in at least one patient.

The protein sequence encoded by BCL2/adenovirus E1B 19 kD-interacting protein 3-like is represented in the public databases by the accession NP_{—}004322 and is described in this patent by SEQ ID NO: 217. The nucleotide sequence is represented in the public sequence databases by the accession NM_{—}004331 and is described in this patent by SEQ ID NO: 218. Hypoxia is an important feature of several diseases, and genes that respond to this stimulus are therefore implicated in the pathogenesis and have utility in the design of therapeutic, prognostic and diagnostic products.

The protein sequence encoded by SLC2A1 is represented in the public databases by the accession NP_{—}006507 and is described in this patent by SEQ ID NO: 219. The nucleotide sequence is represented in the public sequence databases by the accession NM_{—}006516 and is described in this patent by SEQ ID NO: 220. SLC2A1 is a glucose transporter gene and is also known as GLUT1. Hypoxia is an important feature of several diseases, and genes that respond to this stimulus are therefore implicated in the pathogenesis and have utility in the design of therapeutic, prognostic and diagnostic products.

Clone p1P3 represents PDGFB. The protein sequence encoded by PDGFB is represented in the public databases by the accession NP_{—}148937 and is described in this patent by SEQ ID NO: 221. The nucleotide sequence is represented in the public sequence databases by the accession NM_{—}033016 and is described in this patent by SEQ ID NO: 222. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. PDGFB is induced in macrophages activated by LPS and gamma interferon.

Clones p1A8 and p1A9 represent Lactate dehydrogenase A. The protein sequence encoded by Lactate dehydrogenase A is represented in the public databases by the accession NP_{—}005557 and is described in this patent by SEQ ID NO: 223. The nucleotide sequence is represented in the public sequence databases by the accession NM_{—}005566 and is described in this patent by SEQ ID NO: 224. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Lactate dehydrogenase A is repressed in macrophages activated by LPS and gamma interferon and is also repressed in macrophages activated by IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Lactate dehydrogenase A is induced in macrophages activated by TNFalpha. Hypoxia is frequently found in human tumours where macrophage infiltrates are also found. In a series of 5 patients with either ovarian or breast cancer, Lactate dehydrogenase A is up-regulated in the malignant tissue as compared to adjacent normal tissue in at least one patient.

Clone p1B17 represents Tissue factor. The protein sequence encoded by Tissue factor is represented in the public databases by the accession NP_{—}001984 and is described in this patent by SEQ ID NO: 225. The nucleotide sequence is represented in the public sequence databases by the accession NM_{—}001993 and is described in this patent by SEQ ID NO: 226. Hypoxia is an important feature of several diseases, and genes that respond to this stimulus are therefore implicated in the pathogenesis and have utility in the design of therapeutic, prognostic and diagnostic products. Tissue factor is preferentially induced by hypoxia in mammary epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TNFalpha is an inflammatory cytokine, which acts on macrophages, and has been shown to be central to the pathophysiology and treatment of diseases including rheumatoid arthritis. Cienes that change in expression in response to TNFalpha therefore have utility in the design of therapeutic, prognostic and diagnostic products for such inflammatory conditions. Tissue factor is induced in macrophages activated by TNFalpha. Tissue factor is the primary initiator of blood coagulation with structural homology to the cytokine receptor family, and has been implicated in various vascular processes including metastasis, angiogenesis, and atherosclerosis. Our demonstration of hypoxic regulation leads to a clear undertanding of the possibility of intervention in disease by modulation of Tissue factor activity.

Clone p1O20 represents VEGF. The protein sequence encoded by VEGF is represented in the public databases by the accession NP_{—}003367 and is described in this patent by SEQ ID NO: 227. The nucleotide sequence is represented in the public sequence databases by the accession NM_{—}003376 and is described in this patent by SEQ ID NO: 228. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, VEGF is up-regulated in the malignant tissue as compared to adjacent normal tissue in at least one patient.

Clone p1B2 represents N-myc downstream regulated. The protein sequence encoded by N-myc downstream regulated is represented in the public databases by the accession NP_{—}006087 and is described in this patent by SEQ ID NO: 229. The nucleotide sequence is represented in the public sequence databases by the accession NM_{—}006096 and is described in this patent by SEQ ID NO: 230. Hypoxia is an important feature of several diseases, and genes that respond to this stimulus are therefore implicated in the pathogenesis and have utility in the design of therapeutic, prognostic and diagnostic products. N-myc downstream regulated is preferentially induced by hypoxia in mammary epithelial cells.

Clone p1B3 represents Proline 4-hydroxylase, alpha polypeptide I. The protein sequence encoded by Proline 4-hydroxylase, alpha polypeptide I is represented in the public databases by the accession NP_{—}000908 and is described in this patent by SEQ ID NO: 231. The nucleotide sequence is represented in the public sequence databases by the accession NM_{—}000917 and is described in this patent by SEQ ID NO: 232. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Proline 4-hydroxylase, alpha polypeptide I is repressed in macrophages activated by LPS and gamma interferon. Hypoxia is frequently found in human tumours where macrophage infiltrates are also found. In a series of 5 patients with either ovarian or breast cancer, Proline 4-hydroxylase, alpha polypeptide I is down-regulated in the malignant tissue as compared to adjacent normal tissue in at least one patient.

The protein sequence encoded by BCL2/adenovirus E1B-interacting protein 3 is represented in the public databases by the accession NP_{—}004043 and is described in this patent by SEQ ID NO: 233. The nucleotide sequence is represented in the public sequence databases by the accession NM_{—}004052 and is described in this patent by SEQ ID NO: 234. Hypoxia is an important feature of several diseases, and genes that respond to this stimulus are therefore implicated in the pathogenesis and have utility in the design of therapeutic, prognostic and diagnostic products.

Clones p1B18 and p1B19 represent Plasminogen activator inhibitor, type 1. The protein sequence encoded by Plasminogen activator inhibitor, type 1 is represented in the public databases by the accession NP_{—}000593 and is described in this patent by SEQ ID NO: 235. The nucleotide sequence is represented in the public sequence databases by the accession NM_{—}000602 and is described in this patent by SEQ ID NO: 236. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Plasminogen activator inhibitor, type 1 is induced in macrophages activated by LPS and gamma interferon. Plasminogen activator inhibitor, type 1 is repressed in macrophages activated by IL-17. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Plasminogen activator inhibitor, type 1 is induced in macrophages activated by TNFalpha. Hypoxia is frequently found in human tumours where macrophage infiltrates are also found. In a series of 5 patients with either ovarian or breast cancer, Plasminogen activator inhibitor, type 1 is down-regulated in the malignant tissue as compared to adjacent normal tissue in at least one patient.

Clone p1N17 represents COX-2. The protein sequence encoded by COX-2 is represented in the public databases by the accession NP_{—}000954 and is described in this patent by SEQ ID NO: 237. The nucleotide sequence is represented in the public sequence databases by the accession NM_{—}000963 and is described in this patent by SEQ ID NO: 238. Hypoxia is an important feature of several diseases, and genes that respond to this stimulus are therefore implicated in the pathogenesis and have utility in the design of therapeutic, prognostic and diagnostic products. COX-2 is preferentially induced by hypoxia in mammary epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. COX-2 is induced in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. COX-2 is induced in macrophages activated by TNFalpha. In view of the known role of COX-2 in prostaglandin synthesis and tumour progression, its induction by hypoxia has profound clinical implications, and clear utility in diagnosis and therapy. Hypoxia is frequently found in human tumours where macrophage infiltrates are also found.

Clone p1A24 represents Metallothionein 1H. The protein sequence encoded by Metallothionein 1H is represented in the public databases by the accession NP_{—}005942 and is described in this patent by SEQ ID NO: 239. The nucleotide sequence is represented in the public sequence databases by the accession NM_{—}005951 and is described in this patent by SEQ ID NO: 240. Metallothioneins can act as an antioxidant and free-radical scavenger and are therefore protective against cell death in hypoxia. Hypoxia is an important feature of several diseases, and genes that respond to this stimulus are therefore implicated in the pathogenesis and have utility in the design of therapeutic, prognostic and diagnostic products. Hepatocytes are the main cell type of the liver and genes which are induced in response to hypoxia in this cell type are relevant to development of diagnostics and therapeutics towards liver diseases involving hypoxia, including cirrhosis. Metallothionein 1H is preferentially induced by hypoxia in hepatocytes. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Metallothionein 1H is induced in macrophages activated by LPS and gamma interferon.

The protein sequence encoded by Metallothionein 1L is represented in the public databases by the accession NP_{—}002441 and is described in this patent by SEQ ID NO: 241. The nucleotide sequence is represented in the public sequence databases by the accession NM_{—}002450 and is described in this patent by SEQ ID NO: 242. Metallothioneins can act as an antioxidant and free-radical scavenger and are therefore protective against cell death in hypoxia. Hypoxia is an important feature of several diseases, and genes that respond to this stimulus are therefore implicated in the pathogenesis and have utility in the design of therapeutic, prognostic and diagnostic products.

Clone p1B1 represents Metallothionein 1G. The protein sequence encoded by Metallothionein 1G is represented in the public databases by the accession NP_{—}005941 and is described in this patent by SEQ ID NO: 243. The nucleotide sequence is represented in the public sequence databases by the accession NM_{—}005950 and is described in this patent by SEQ ID NO: 244. Metallothioneins can act as an antioxidant and free-radical scavenger and are therefore protective against cell death in hypoxia. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors which mediate the response to hypoxia of several genes, and have them selves been implicated in specific diseases. By adenoviral over-expression of HIF1alpha we show augmentation of the hypoxic induction of certain genes, further confirming their status as responsive to hypoxia. Metallothionein 1G has been shown to be induced by hypoxia to a greater degree following adenoviral over-expression of HIF1alpha. Hepatocytes are the main cell type of the liver and genes which are induced in response to hypoxia in this cell type are relevant to development of diagnostics and therapeutics towards liver diseases involving hypoxia, including cirrhosis. Metallothionein 1G is preferentially induced by hypoxia in hepatocytes. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Metallothionein 1G is induced in macrophages activated by LPS and gamma interferon. Hypoxia is frequently found in human tumours where macrophage infiltrates are also found. In a series of 5 patients with either ovarian or breast cancer, Metallothionein 1G is up-regulated in the malignant tissue as compared to adjacent normal tissue in at least one patient.

The protein sequence encoded by Metallothionein 1E (functional) is not represented in the public databases by a protein accession. The nucleotide sequence is represented in the public sequence databases by the accession AA872383 and is described in this patent by SEQ ID NO: 246. Metallothioneins can act as an antioxidant and free-radical scavenger and are therefore protective against cell death in hypoxia. Hypoxia is an important feature of several diseases, and genes that respond to this stimulus are therefore implicated in the pathogenesis and have utility in the design of therapeutic, prognostic and diagnostic products.

Clones p1A1, p1A2, p1A3 and p1A4 represent SLC2A3. The protein sequence encoded by SLC2A3 is represented in the public databases by the accession NP_{—}008862 and is described in this patent by SEQ ID NO: 247. The nucleotide sequence is represented in the public sequence databases by the accession NM_{—}006931 and is described in this patent by SEQ ID NO: 248. Hypoxia is an important feature of several diseases, and genes that respond to this stimulus are therefore implicated in the pathogenesis and have utility in the design of therapeutic, prognostic and diagnostic products. SLC2A3 is induced in macrophages treated with the inhibitory cytokine IL-10. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. SLC2A3 is induced in macrophages activated by TNFalpha.

Clones p1A15, p1A16, p1A17 and p1A18 represent Hexokinase-2. The protein sequence encoded by Hexokinase-2 is represented in the public databases by the accession NP_{—}000180 and is described in this patent by SEQ ID NO: 249. The nucleotide sequence is represented in the public sequence databases by the accession NM_{—}000189 and is described in this patent by SEQ ID NO: 250. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hexokinase-2 is repressed in macrophages activated by LPS and gamma interferon.

Clones p1B14, p1B15 and p1B16 represent Interleukin 8. The protein sequence encoded by Interleukin 8 is represented in the public databases by the accession NP_{—}000575 and is described in this patent by SEQ ID NO: 251. The nucleotide sequence is represented in the public sequence databases by the accession NM_{—}000584 and is described in this patent by SEQ ID NO: 252. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Interleukin 8 is induced in macrophages activated by LPS and gamma interferon and is also induced in macrophages activated by IL-17. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Interleukin 8 is induced in macrophages activated by TNFalpha. Hypoxia is frequently found in human tumours where macrophage infiltrates are also found. In a series of 5 patients with either ovarian or breast cancer, Interleukin 8 is down-regulated in the malignant tissue as compared to adjacent normal tissue in at least one patient.

Clones p1A11 and p1A12 represent GAPDH. The protein sequence encoded by GAPDH is represented in the public databases by the accession NP_{—}002037 and is described in this patent by SEQ ID NO: 253. The nucleotide sequence is represented in the public sequence databases by the accession NM_{—}002046 and is described in this patent by SEQ ID NO: 254. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. GAPDH is repressed in macrophages activated by LPS and gamma interferon and is also induced in macrophages activated by IL-17 or IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. GAPDH is induced in macrophages activated by TNFalpha. Hypoxia is frequently found in human tumours where macrophage infiltrates are also found. In a series of 5 patients with either ovarian or breast cancer, GAPDH is up-regulated in the malignant tissue as compared to adjacent normal tissue in at least one patient.

Clone p1A13 represents Phosphoglycerate kinase 1. The protein sequence encoded by Phosphoglycerate kinase 1 is represented in the public databases by the accession NP_{—}000282 and is described in this patent by SEQ ID NO: 255. The nucleotide sequence is represented in the public sequence databases by the accession NM_{—}000291 and is described in this patent by SEQ ID NO: 256. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Phosphoglycerate kinase 1 is repressed in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Phosphoglycerate kinase 1 is induced in macrophages activated by TNFalpha.

Clone p1A14 represents Enolase 1. The protein sequence encoded by Enolase 1 is represented in the public databases by the accession NP_{—}001419 and is described in this patent by SEQ ID NO: 257. The nucleotide sequence is represented in the public sequence databases by the accession NM_{—}001428 and is described in this patent by SEQ ID NO: 258. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Enolase 1 is repressed in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Enolase 1 is induced in macrophages activated by TNFalpha. Hypoxia is frequently found in human tumours where macrophage infiltrates are also found. In a series of 5 patients with either ovarian or breast cancer, Enolase 1 is up-regulated in the malignant tissue as compared to adjacent normal tissue in at least one patient.

Clone p1A19 represents Aldolase C. The protein sequence encoded by Aldolase C is represented in the public databases by the accession NP_{—}005156 and is described in this patent by SEQ ID NO: 259. The nucleotide sequence is represented in the public sequence databases by the accession NM_{—}005165 and is described in this patent by SEQ ID NO: 260. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Aldolase C is induced in macrophages activated by IL-15. Aldolase C is repressed in macrophages activated by IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Aldolase C is induced in macrophages activated by TNFalpha. Hypoxia is frequently found in human tumours where macrophage infiltrates are also found. In a series of 5 patients with either ovarian or breast cancer, Aldolase is up-regulated in the malignant tissue as compared to adjacent normal tissue in at least one patient.

Clone p1A20 represents Triosephosphate isomerase 1. The protein sequence encoded by Triosephosphate isomerase 1 is represented in the public databases by the accession NP_{—}000356 and is described in this patent by SEQ ID NO: 261. The nucleotide sequence is represented in the public sequence databases by the accession NM_{—}000365 and is described in this patent by SEQ ID NO: 262. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Triosephosphate isomerase 1 is repressed in macrophages activated by LPS and gamma interferon and is also repressed in macrophages activated by IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Triosephosphate isomerase 1 is induced in macrophages activated by TNFalpha.

Clone p1A23 represents Metallothionein 2A. The protein sequence encoded by Metallothionein 2A is represented in the public databases by the accession NP_{—}005944 and is described in this patent by SEQ ID NO: 265. The nucleotide sequence is represented in the public sequence databases by the accession NM_{—}005953 and is described in this patent by SEQ ID NO: 266. Metallothioneins can act as an antioxidant and free-radical scavenger and are therefore protective against cell death in hypoxia. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors which mediate the response to hypoxia of several genes, and have them selves been implicated in specific diseases. By adenoviral over-expression of HIF1alpha we show augmentation of the hypoxic induction of certain genes, further confirming their status as responsive to hypoxia. Metallothionein 2A has been shown to be induced by hypoxia to a greater degree following adenoviral over-expression of HIF1alpha. Hepatocytes are the main cell type of the liver and genes which are induced in response to hypoxia in this cell type are relevant to development of diagnostics and therapeutics towards liver diseases involving hypoxia, including cirrhosis. Metallothionein 2A is preferentially induced by hypoxia in hepatocytes. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Metallothionein 2A is induced in macrophages activated by LPS and gamma interferon and also induced in macrophages activated by IL-15. Hypoxia is frequently found in human tumours where macrophage infiltrates are also found. In a series of 5 patients with either ovarian or breast cancer, Metallothionein 2A is down-regulated in the malignant tissue as compared to adjacent normal tissue in at least one patient.

Clones p1B20 and p1B21 represent Osteopontin. The protein sequence encoded by Osteopontin is represented in the public databases by the accession NP_{—}000573 and is described in this patent by SEQ ID NO: 267. The nucleotide sequence is represented in the public sequence databases by the accession NM_{—}000582 and is described in this patent by SEQ ID NO: 268. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Osteopontin is preferentially induced by hypoxia in monocytes or macrophages and a restricted number of other cell types. Osteopontin has been shown recently to play a role in autoimmune disease [Chabas et al, 2001, Science 294: 1731-5]. We present a new association between the hypoxic response and autoimmune disease. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Osteopontin is repressed in macrophages activated by LPS and gamma interferon. Hypoxia is frequently found in human tumours where macrophage infiltrates are also found. In a series of 5 patients with either ovarian or breast cancer, Osteopontin is up-regulated in the malignant tissue as compared to adjacent normal tissue in at least one patient.

Clones p1C17 and p1C18 represent Granulin. The protein sequence encoded by Granulin is represented in the public databases by the accession NP_{—}002078 and is described in this patent by SEQ ID NO: 269. The nucleotide sequence is represented in the public sequence databases by the accession NM_{—}002087 and is described in this patent by SEQ ID NO: 270. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Granulin is repressed in macrophages activated by LPS and gamma interferon. Hypoxia is frequently found in human tumours where macrophage infiltrates are also found. In a series of 5 patients with either ovarian or breast cancer, Granulin is up-regulated in the malignant tissue as compared to adjacent normal tissue in at least one patient. The up-regulation of Granulin, which is a known growth factor, is a clinically significant feature of the hypoxic response with clear diagnostic and therapeutic utility.

Clone p1D8 represents Hypoxia-inducible protein 2. The protein sequence encoded by Hypoxia-inducible protein 2 is represented in the public databases by the accession NP_{—}037464 and is described in this patent by SEQ ID NO: 271. The nucleotide sequence is represented in the public sequence databases by the accession NM_{—}013332 and is described in this patent by SEQ ID NO: 272. Hypoxia is an important feature of several diseases, and genes that respond to this stimulus are therefore implicated in the pathogenesis and have utility in the design of therapeutic, prognostic and diagnostic products. Hypoxia-inducible protein 2 is induced in macrophages treated with the inhibitory cytokine IL-10. Hypoxia-inducible protein 2 is repressed in macrophages activated by IL-17 and is also repressed in macrophages activated by IL-15.

Clone p1A10 represents Enolase 2. The protein sequence encoded by Enolase 2 is represented in the public databases by the accession NP_{—}001966 and is described in this patent by SEQ ID NO: 273. The nucleotide sequence is represented in the public sequence databases by the accession NM_{—}001975 and is described in this patent by SEQ ID NO: 274. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Enolase 2 is repressed in macrophages activated by LPS and gamma interferon. Hypoxia is frequently found in human tumours where macrophage infiltrates are also found. In a series of 5 patients with either ovarian or breast cancer, Enolase 2 is up-regulated in the malignant tissue as compared to adjacent normal tissue in at least one patient. We expect it to have an anti-inflammatory role.

Clone p1G24 represents Glycogen synthase 1. The protein sequence encoded by Glycogen synthase 1 is represented in the public databases by the accession NP_{—}002094 and is described in this patent by SEQ ID NO: 275. The nucleotide sequence is represented in the public sequence databases by the accession NM_{—}002103 and is described in this patent by SEQ ID NO: 276. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Glycogen synthase 1 is repressed in macrophages activated by IL-17 and is also repressed in macrophages activated by IL-15.

Clone p1G23 represents ALCAM. The protein sequence encoded by ALCAM is represented in the public databases by the accession NP_{—}001618 and is described in this patent by SEQ ID NO: 277. The nucleotide sequence is represented in the public sequence databases by the accession NM_{—}001627 and is described in this patent by SEQ ID NO: 278. Hypoxia is an important feature of several diseases, and genes that respond to this stimulus are therefore implicated in the pathogenesis and have utility in the design of therapeutic, prognostic and diagnostic products. In view of the recently-discovered role of ALCAM in angiogenesis [Ohneda et al, 2001, Blood 2001 October 1;98(7):2134-42], our demonstration of hypoxic regulation of ALCAM has great clinical significance in the treatment and diagnosis of vascular disease and cancer.

Clone p1G7 represents an unannotated EST. The protein sequence encoded by this EST is not represented in the public databases by a protein accession. The nucleotide sequence is represented in the public sequence databases by the accession BC008022 and is described in this patent by SEQ ID NO: 282. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The EST represented by SEQ ID NO: 282 is repressed in macrophages activated by LPS and gamma interferon. We expect the product of EST represented by SEQ ID NO: 282 to have an anti-inflammatory role.

Clone p2A23 represents Chitinase 3-like 2. The protein sequence encoded by Chitinase 3-like 2 is represented in the public databases by the accession NP_{—}003991 and is described in this patent by SEQ ID NO: 283. The nucleotide sequence is represented in the public sequence databases by the accession NM_{—}004000 and is described in this patent by SEQ ID NO: 284. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, Chitinase 3-like 2 is down-regulated in the malignant tissue as compared to adjacent normal tissue in at least one patient.

Clone p1G1 represents BACH1. The protein sequence encoded by BACH1 is represented in the public databases by the accession NP_{—}001177 and is described in this patent by SEQ ID NO: 285. The nucleotide sequence is represented in the public sequence databases by the accession NM_{—}001186 and is described in this patent by SEQ ID NO: 286. BACH1, a novel helicase-like protein, interacts directly with BRCA1 and contributes to its DNA repair function. Hypoxia is an important feature of several diseases, and genes that respond to this stimulus are therefore implicated in the pathogenesis and have utility in the design of therapeutic, prognostic and diagnostic products. The induction by hypoxia of this known transcriptional repressor and potential oncogene [Cantor et al 2001, Cell 105:149-60] is a very significant finding with profound implications for the diagnosis and treatment of cancer.

Clone p1G15 represents Phosphoglucomutase 1. The protein sequence encoded by Phosphoglucomutase 1 is represented in the public databases by the accession NP_{—}002624 and is described in this patent by SEQ ID NO: 287. The nucleotide sequence is represented in the public sequence databases by the accession NM_{—}002633 and is described in this patent by SEQ ID NO: 288. Hypoxia is an important feature of several diseases, and genes that respond to this stimulus are therefore implicated in the pathogenesis and have utility in the design of therapeutic, prognostic and diagnostic products. Phosphoglucomutase 1 is induced in macrophages treated with the inhibitory cytokine IL-10.

Clone p1F23 represents hypothetical protein LOC51014. The protein sequence encoded by hypothetical protein LOC51014 is represented in the public databases by the accession Q9Y3B3 and is described in this patent by SEQ ID NO: 289. The nucleotide sequence is represented in the public sequence databases by the accession AF151867 and is described in this patent by SEQ ID NO: 290. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, hypothetical protein LOC51014 is down-regulated in the malignant tissue as compared to adjacent normal tissue in at least one patient.

Clone p1G8 represents Sin3-associated polypeptide. The protein sequence encoded by Sin3-associated polypeptide is represented in the public databases by the accession NP_{—}003855 and is described in this patent by SEQ ID NO: 291. The nucleotide sequence is represented in the public sequence databases by the accession NM_{—}003864 and is described in this patent by SEQ ID NO: 292. Hypoxia is an important feature of several diseases, and genes that respond to this stimulus are therefore implicated in the pathogenesis and have utility in the design of therapeutic, prognostic and diagnostic products.

Clone p1G13 represents ABCA1. The protein sequence encoded by ABCA1 is represented in the public databases by the accession NP_{—}005493 and is described in this patent by SEQ ID NO: 293. The nucleotide sequence is represented in the public sequence databases by the accession NM_{—}005502 and is described in this patent by SEQ ID NO: 294. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. ABCA1 is repressed in macrophages activated by LPS and gamma interferon. The hypoxia induction of ABCA1, which is known to be relevant to atherosclerosis [Kielar et al 2001, Clin Chem 47:2089-97], has profound implications for the diagnosis and treatment of this disease.

Clone p1G10 represents SEC24 member A. The protein sequence encoded by SEC24 member A is represented in the public databases by the accession CAA10334 and is described in this patent by SEQ ID NO: 295. The nucleotide sequence is represented in the public sequence databases by the accession AJ131244 and is described in this patent by SEQ ID NO: 296. Hypoxia is an important feature of several diseases, and genes that respond to this stimulus are therefore implicated in the pathogenesis and have utility in the design of therapeutic, prognostic and diagnostic products.

Clone p1F24 represents Glia-derived nexin. The protein sequence encoded by Glia-derived nexin is represented in the public databases by the accession AAA35883 and is described in this patent by SEQ ID NO: 297. The nucleotide sequence is represented in the public sequence databases by the accession M17783 and is described in this patent by SEQ ID NO: 298. Glia-derived nexin is a glycoprotein that functions as a serine protease inhibitor with activity towards thrombin, trypsin and urokinase. It is known to play a role in neuro-degeneration [Seidel et al 1998, Brain Res Mol Brain Res 60:296-300]. Thus the hypoxia induction of this gene is highly significant for the diagnosis and treatment of neuro-degenerative disease. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Glia-derived nexin is induced in macrophages activated by LPS and gamma interferon. Hypoxia is frequently found in human tumours where macrophage infiltrates are also found. In a series of 5 patients with either ovarian or breast cancer, Glia-derived nexin is down-regulated in the malignant tissue as compared to adjacent normal tissue in at least one patient.

Clone p1G2 represents Postsynaptic density-95. The protein sequence encoded by Postsynaptic density-95 is represented in the public databases by the accession NP_{—}001356 and is described in this patent by SEQ ID NO: 299. The nucleotide sequence is represented in the public sequence databases by the accession NM_{—}001365 and is described in this patent by SEQ ID NO: 300. Postsynaptic density-95 belongs to the MAGUK family of cell junction proteins. Hypoxia is an important feature of several diseases, and genes that respond to this stimulus are therefore implicated in the pathogenesis and have utility in the design of therapeutic, prognostic and diagnostic products. The recent demonstration for a possible role for Postsynaptic density-95 in ischaemic pre-conditioning [Tauskela et al 2001, Neuroscience 107:571-584] underlines the medical significance of our determination of the hypoxic regulation of this gene, and its utility in the diagnosis and treatment of ischaemic disease.

Clone p1G11 represents Tumour protein D52. The protein sequence encoded by Tumour protein D52 is represented in the public databases by the accession NP_{—}005070 and is described in this patent by SEQ ID NO: 301. The nucleotide sequence is represented in the public sequence databases by the accession NM_{—}005079 and is described in this patent by SEQ ID NO: 302. Hypoxia is an important feature of several diseases, and genes that respond to this stimulus are therefore implicated in the pathogenesis and have utility in the design of therapeutic, prognostic and diagnostic products. The response of renal epithelial cells to hypoxia is pertinent to kidney failure, especially regarding the medullary tissue. Tumour protein D52 is preferentially induced by hypoxia in renal epithelial cells. Hypoxia is frequently found in human tumours where macrophage infiltrates are also found. In a series of 5 patients with either ovarian or breast cancer, Tumour protein D52 is up-regulated in the malignant tissue as compared to adjacent normal tissue in at least one patient. Our observation of hypoxia-regulation of this tumour-associated protein is highly significant for the diagnosis and treatment of cancer.

Clone p1G16 represents p27, Kip1. The protein sequence encoded by p27, Kip1is represented in the public databases by the accession NP_{—}004055 and is described in this patent by SEQ ID NO: 303. The nucleotide sequence is represented in the public sequence databases by the accession NM_{—}004064 and is described in this patent by SEQ ID NO: 304. Hypoxia is an important feature of several diseases, and genes that respond to this stimulus are therefore implicated in the pathogenesis and have utility in the design of therapeutic, prognostic and diagnostic products. The hypoxia regulation of this anti-mitogen has important utility in oncology and angiogenesis [Fouty et al 2001, Am J Respir Cell Mol Biol 25:652-658].

Clone p1G9 represents PI-3-kinase, catalytic, beta polypeptide. The protein sequence encoded by PI-3-kinase, catalytic, beta polypeptide is represented in the public databases by the accession NP_{—}006210 and is described in this patent by SEQ ID NO: 305. The nucleotide sequence is represented in the public sequence databases by the accession NM_{—}006219 and is described in this patent by SEQ ID NO: 306. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. PI-3-kinase, catalytic, beta polypeptide is repressed in macrophages activated by LPS and gamma interferon. The very recent publication of a role for PI3 kinase in angiogenesis induced by hypoxic pre-conditioning [Zhu et al 2001, FEBS Lett 508:369-74] re-enforces the clinical utility which we claim for this gene as a result of its hypoxia-induction.

Clone p1G4 represents SLC5A3. The protein sequence encoded by SLC5A3 is represented in the public databases by the accession AAC39548 and is described in this patent by SEQ ID NO: 307. The nucleotide sequence is represented in the public sequence databases by the accession AF027153 and is described in this patent by SEQ ID NO: 308. Hypoxia is an important feature of several diseases, and genes that respond to this stimulus are therefore implicated in the pathogenesis and have utility in the design of therapeutic, prognostic and diagnostic products. SLC5A3 is over-expressed in the brains of children with Downs Syndrome, and may play a role in brain pathology [Berry et al 1999, J Pediatr 135:94-7]. Thus our claims of clinical utility following from hypoxia induction have great medical significance for the diagnosis and treatment of ischaemic and degenerative disease. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SLC5A3 is repressed in macrophages activated by LPS and gamma interferon.

Clone p1G14 represents Cytohesin binding protein. The protein sequence encoded by Cytohesin binding protein is represented in the public databases by the accession NP_{—}004279 and is described in this patent by SEQ ID NO: 309. The nucleotide sequence is represented in the public sequence databases by the accession NM_{—}004288 and is described in this patent by SEQ ID NO: 310. Hypoxia is an important feature of several diseases, and genes that respond to this stimulus are therefore implicated in the pathogenesis and have utility in the design of therapeutic, prognostic and diagnostic products. Cytohesin has been shown to modulate PI3-kinase activity [Dierks et al 2001, J Biol Chem 276:37472-81], re-enforcing our claim here and elsewhere in this filing of the relevance to the hypoxic response of pathways controlled by the critical second-messenger PI3.

Clones p1A5 and p1A6 represent SLC2A5. The protein sequence encoded by SLC2A5 is represented in the public databases by the accession NP_{—}003030 and is described in this patent by SEQ ID NO: 311. The nucleotide sequence is represented in the public sequence databases by the accession NM_{—}003039 and is described in this patent by SEQ ID NO: 312. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SLC2A5 is repressed in macrophages activated by LPS and gamma interferon. Hypoxia is frequently found in human tumours where macrophage infiltrates are also found. In a series of 5 patients with either ovarian or breast cancer, SLC2A5 is up-regulated in the malignant tissue as compared to adjacent normal tissue in at least one patient.

Clones p1B6, p1B7, p1B8 and p1B9 represent Adipophilin. The protein sequence encoded by Adipophilin is represented in the public databases by the accession NP_{—}001113 and is described in this patent by SEQ ID NO: 313. The nucleotide sequence is represented in the public sequence databases by the accession NM_{—}001122 and is described in this patent by SEQ ID NO: 314. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. The hypoxia induction of adipophilin has profound implications for the causation, diagnosis and treatment of atherosclerosis, because this protein plays a key role in the uptake of lipid and foam cell formation [Buechler et al 2001, Biochim Biophys Acta 1532:97-104]. Adipophilin is preferentially induced by hypoxia in monocytes or macrophages and a restricted number of other cell types. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Adipophilin is repressed in macrophages activated by LPS and gamma interferon and is also repressed in macrophages activated by IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Adipophilin is induced in macrophages activated by TNFalpha. Hypoxia is frequently found in human tumours where macrophage infiltrates are also found. In a series of 5 patients with either ovarian or breast cancer, Adipophilin is up-regulated and also down-regulated in the malignant tissue as compared to adjacent normal tissue in at least one patient.

Clone p1G17 represents early development regulator 2. The protein sequence encoded by early development regulator 2 is represented in the public databases by the accession NP_{—}004418 and is described in this patent by SEQ ID NO: 315. The nucleotide sequence is represented in the public sequence databases by the accession NM_{—}004427 and is described in this patent by SEQ ID NO: 316. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Early development regulator 2 is repressed in macrophages activated by LPS and gamma interferon.

Clone p1G3 represents B-cell translocation gene 1. The protein sequence encoded by B-cell translocation gene 1 is represented in the public databases by the accession NP_{—}001722 and is described in this patent by SEQ ID NO: 317. The nucleotide sequence is represented in the public sequence databases by the accession NM_{—}001731 and is described in this patent by SEQ ID NO: 318. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. B-cell translocation gene 1 is induced in macrophages activated by LPS and gamma interferon. Hypoxia is frequently found in human tumours where macrophage infiltrates are also found. In a series of 5 patients with either ovarian or breast cancer, B-cell translocation gene 1 is down-regulated in the malignant tissue as compared to adjacent normal tissue in at least one patient.

Clone p1F22 represents Sorting nexin 9. The protein sequence encoded by Sorting nexin 9 is represented in the public databases by the accession NP_{—}057308 and is described in this patent by SEQ ID NO: 319. The nucleotide sequence is represented in the public sequence databases by the accession NM_{—}016224 and is described in this patent by SEQ ID NO: 320. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Sorting nexin 9 is induced in macrophages activated by TNFalpha.

Clone p1G12 represents Cyclin G2. The protein sequence encoded by Cyclin G2 is represented in the public databases by the accession NP_{—}004345 and is described in this patent by SEQ ID NO: 321. The nucleotide sequence is represented in the public sequence databases by the accession NM_{—}004354 and is described in this patent by SEQ ID NO: 322. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Cyclin G2 is repressed in macrophages activated by LPS and gamma interferon.

Clone p1F11 represents hypothetical protein LOC51754. The protein sequence encoded by hypothetical protein LOC51754 is represented in the public databases by the accession XP_{—}049657 and is described in this patent by SEQ ID NO: 323. The nucleotide sequence is represented in the public sequence databases by the accession AL137430 and is described in this patent by SEQ ID NO: 324. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein LOC51754 is repressed in macrophages activated by LPS and gamma interferon. Hypoxia is frequently found in human tumours where macrophage infiltrates are also found. In a series of 5 patients with either ovarian or breast cancer, hypothetical protein LOC51754 is up-regulated in the malignant tissue as compared to adjacent normal tissue in at least one patient.

Clone p1F14 represents Butyrate response factor 1. The protein sequence encoded by Butyrate response factor 1 is represented in the public databases by the accession NP_{—}004917 and is described in this patent by SEQ ID NO: 327. The nucleotide sequence is represented in the public sequence databases by the accession NM_{—}004926 and is described in this patent by SEQ ID NO: 328. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors which mediate the response to hypoxia of several genes, and have them selves been implicated in specific diseases. By adenoviral over-expression of EPAS1 we show augmentation of the hypoxic induction of certain genes, further confirming their status as responsive to hypoxia. Butyrate response factor 1 has been shown to be induced by hypoxia to a greater degree following adenoviral over-expression of EPAS 1. Hypoxia is frequently found in human tumours where macrophage infiltrates are also found. In a series of 5 patients with either ovarian or breast cancer, Butyrate response factor 1 is down-regulated in the malignant tissue as compared to adjacent normal tissue in at least one patient.

Clone p1F17 represents P8 protein (candidate of metastasis 1). The protein sequence encoded by P8 protein (candidate of metastasis 1) is represented in the public databases by the accession NP_{—}036517 and is described in this patent by SEQ ID NO: 329. The nucleotide sequence is represented in the public sequence databases by the accession NM_{—}012385 and is described in this patent by SEQ ID NO: 330. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. P8 protein (candidate of metastasis 1) is induced in macrophages activated by LPS and gamma interferon. Hypoxia is frequently found in human tumours where macrophage infiltrates are also found. In a series of 5 patients with either ovarian or breast cancer, P8 protein (candidate of metastasis 1) is up-regulated and also down-regulated in the malignant tissue as compared to adjacent normal tissue in at least one patient.

Clones p1C1 and p1C2 represent CXCR4. The protein sequence encoded by CXCR4 is represented in the public databases by the accession NP_{—}003458 and is described in this patent by SEQ ID NO: 331. The nucleotide sequence is represented in the public sequence databases by the accession NM_{—}003467 and is described in this patent by SEQ ID NO: 332. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. CXCR4 is repressed in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. CXCR4 is induced in macrophages activated by TNFalpha. CXCR4 may act through the PI3-K pathway. Hypoxia is frequently found in human tumours where macrophage infiltrates are also found. In a series of 5 patients with either ovarian or breast cancer, CXCR4 is up-regulated and also down-regulated in the malignant tissue as compared to adjacent normal tissue in at least one patient.

Clone p1F3 represents hypothetical protein XP_{—}017131. The protein sequence encoded by hypothetical protein XP_{—}017131 is represented in the public databases by the accession XP_{—}017131 and is described in this patent by SEQ ID NO: 333. The nucleotide sequence is represented in the public sequence databases by the accession XM_{—}017131 and is described in this patent by SEQ ID NO: 334. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein XP_{—}017131 is repressed in macrophages activated by LPS and gamma interferon. Hypoxia is frequently found in human tumours where macrophage infiltrates are also found. In a series of 5 patients with either ovarian or breast cancer, hypothetical protein XP_{—}017131 is down-regulated in the malignant tissue as compared to adjacent normal tissue in at least one patient.

Clone p1F20 represents Proline-rich protein with nuclear targeting signal. The protein sequence encoded by Proline-rich protein with nuclear targeting signal is represented in the public databases by the accession NP_{—}006804 and is described in this patent by SEQ ID NO: 335. The nucleotide sequence is represented in the public sequence databases by the accession NM_{—}006813 and is described in this patent by SEQ ID NO: 336. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Proline-rich protein with nuclear targeting signal is induced in macrophages activated by TNFalpha. Hypoxia is frequently found in human tumours where macrophage infiltrates are also found. In a series of 5 patients with either ovarian or breast cancer, Proline-rich protein with nuclear targeting signal is down-regulated in the malignant tissue as compared to adjacent normal tissue in at least one patient.

Clone p1F6 represents hypothetical protein hqp0376. The protein sequence encoded by hypothetical protein hqp0376 is represented in the public databases by the accession T08745 and is described in this patent by SEQ ID NO: 337. The nucleotide sequence is represented in the public sequence databases by the accession AF078844 and is described in this patent by SEQ ID NO: 338. Hypothetical protein hqp0376 is a putative dead box protein as it shows high structural similarity to dead box proteins and yeast initiation factor 4A. Hypoxia is an important feature of several diseases, and genes that respond to this stimulus are therefore implicated in the pathogenesis and have utility in the design of therapeutic, prognostic and diagnostic products. HIF1alpha and EPAS1 are transcription factors which mediate the response to hypoxia of several genes, and have them selves been implicated in specific diseases. By adenoviral over-expression of HIF1alpha we show augmentation of the hypoxic induction of certain genes, further confirming their status as responsive to hypoxia. Hypothetical protein hqp0376 has been shown to be induced by hypoxia to a greater degree following adenoviral over-expression of HIF1alpha. Hepatocytes are the main cell type of the liver and genes which are induced in response to hypoxia in this cell type are relevant to development of diagnostics and therapeutics towards liver diseases involving hypoxia, including cirrhosis. Hypothetical protein hqp0376 is preferentially induced by hypoxia in hepatocytes. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Hypothetical protein hqp0376 is induced in macrophages activated by LPS and gamma interferon.

Clone p1F4 represents CYP1. The protein sequence encoded by CYP1 is represented in the public databases by the accession NP_{—}000776 and is described in this patent by SEQ ID NO: 339. The nucleotide sequence is represented in the public sequence databases by the accession NM_{—}000785 and is described in this patent by SEQ ID NO: 340. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. CYP1 is preferentially induced by hypoxia in monocytes or macrophages. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. CYP1 is induced in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. CYP1 is induced in macrophages activated by TNFalpha.

Clone p1F15 represents SHB adaptor protein. The protein sequence encoded by SHB adaptor protein is represented in the public databases by the accession NP_{—}003019 and is described in this patent by SEQ ID NO: 341. The nucleotide sequence is represented in the public sequence databases by the accession NM_{—}003028 and is described in this patent by SEQ ID NO: 342. Hypoxia is an important feature of several diseases, and genes that respond to this stimulus are therefore implicated in the pathogenesis and have utility in the design of therapeutic, prognostic and diagnostic products. SHB adaptor protein participates in tyrosine kinase-mediated signalling and the regulation of angiogenesis and apotosis [Dixelius J. 2000, Blood 95:3403-11]. Our surprising observation of the hypoxia regulation of this protein has clear medical utility in the diagnosis and treatment of vascular disease and cancer. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SHB adaptor protein is repressed in macrophages activated by LPS and gamma interferon.

Clone p1F13 represents Papillomavirus regulatory factor PRF-1. The protein sequence encoded by Papillomavirus regulatory factor PRF-1 is represented in the public databases by the accession NP_{—}061130 and is described in this patent by SEQ ID NO: 343. The nucleotide sequence is represented in the public sequence databases by the accession AK023418 and is described in this patent by SEQ ID NO: 344. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Papillomavirus regulatory factor PRF-1 is repressed in macrophages activated by LPS and gamma interferon.

Clone p1A7 represents SLC31A2. The protein sequence encoded by SLC31A2 is represented in the public databases by the accession NP_{—}001851 and is described in this patent by SEQ ID NO: 345. The nucleotide sequence is represented in the public sequence databases by the accession NM_{—}001860 and is described in this patent by SEQ ID NO: 346. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SLC31A2 is induced in macrophages activated by LPS and gamma interferon.

Clone p1A21 represents UDP-glucose pyrophosphorylase 2. The protein sequence encoded by UDP-glucose pyrophosphorylase 2 is represented in the public databases by the accession NP_{—}006750 and is described in this patent by SEQ ID NO: 347. The nucleotide sequence is represented in the public sequence databases by the accession NM_{—}006759 and is described in this patent by SEQ ID NO: 348. Hypoxia is an important feature of several diseases, and genes that respond to this stimulus are therefore implicated in the pathogenesis and have utility in the design of therapeutic, prognostic and diagnostic products.

Clones p1B4 and p1B5 represent Proline 4-hydroxylase, alpha polypeptide II. The protein sequence encoded by Proline 4-hydroxylase, alpha polypeptide II is represented in the public databases by the accession NP_{—}004190 and is described in this patent by SEQ ID NO: 349. The nucleotide sequence is represented in the public sequence databases by the accession NM_{—}004199 and is described in this patent by SEQ ID NO: 350. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Proline 4-hydroxylase, alpha polypeptide II is repressed in macrophages activated by LPS and gamma interferon and is also repressed in macrophages activated by IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Proline 4-hydroxylase, alpha polypeptide II is induced in macrophages activated by TNFalpha. Hypoxia is frequently found in human tumours where macrophage infiltrates are also found. In a series of 5 patients with either ovarian or breast cancer, Proline 4-hydroxylase, alpha polypeptide II is up-regulated in the malignant tissue as compared to adjacent normal tissue in at least one patient.

Clones p1B10, p1B11 and p1B12 represent Stearoyl-CoA desaturase. The protein sequence encoded by Stearoyl-CoA desaturase is represented in the public databases by the accession NP_{—}005054 and is described in this patent by SEQ ID NO: 351. The nucleotide sequence is represented in the public sequence databases by the accession NM_{—}005063 and is described in this patent by SEQ ID NO: 352. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Stearoyl-CoA desaturase is induced in macrophages activated by TNFalpha.

Clone p1B13 represents Diacylglycerol kinase, zeta. The protein sequence encoded by Diacylglycerol kinase, zeta is represented in the public databases by the accession NP_{—}003637 and is described in this patent by SEQ ID NO: 353. The nucleotide sequence is represented in the public sequence databases by the accession NM_{—}003646 and is described in this patent by SEQ ID NO: 354. Hypoxia is an important feature of several diseases, and genes that respond to this stimulus are therefore implicated in the pathogenesis and have utility in the design of therapeutic, prognostic and diagnostic products.

Clone p1B22 represents Protease, serine, 11. The protein sequence encoded by Protease, serine, 11 is represented in the public databases by the accession NP_{—}002766 and is described in this patent by SEQ ID NO: 355. The nucleotide sequence is represented in the public sequence databases by the accession NM_{—}002775 and is described in this patent by SEQ ID NO: 356. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Protease, serine, 11 is repressed in macrophages activated by LPS and gamma interferon. Hypoxia is frequently found in human tumours where macrophage infiltrates are also found. In a series of 5 patients with either ovarian or breast cancer, Protease, serine, 11 is down-regulated in the malignant tissue as compared to adjacent normal tissue in at least one patient.

Clone p1B23 represents Interleukin 1 receptor antagonist. The protein sequence encoded by Interleukin 1 receptor antagonist is represented in the public databases by the accession NP_{—}000568 and is described in this patent by SEQ ID NO: 357. The nucleotide sequence is represented in the public sequence databases by the accession NM_{—}000577 and is described in this patent by SEQ ID NO: 358. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Interleukin 1 receptor antagonist is preferentially induced by hypoxia in monocytes or macrophages. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflamnmatory conditions. Interleukin 1 receptor antagonist is induced in macrophages activated by TNFalpha.

Clone p1B24 represents NS1-binding protein. The protein sequence encoded by NS1-binding protein is represented in the public databases by the accession NP_{—}006460 and is described in this patent by SEQ ID NO: 359. The nucleotide sequence is represented in the public sequence databases by the accession NM_{—}006469 and is described in this patent by SEQ ID NO: 360. Hypoxia is an important feature of several diseases, and genes that respond to this stimulus are therefore implicated in the pathogenesis and have utility in the design of therapeutic, prognostic and diagnostic products.

Clone p1C3 represents Activin A receptor, type I. The protein sequence encoded by Activin A receptor, type I is represented in the public databases by the accession NP_{—}001096 and is described in this patent by SEQ ID NO: 361. The nucleotide sequence is represented in the public sequence databases by the accession NM_{—}001105 and is described in this patent by SEQ ID NO: 362. Hypoxia is an important feature of several diseases, and genes that respond to this stimulus are therefore implicated in the pathogenesis and have utility in the design of therapeutic, prognostic and diagnostic products. Activin A is known to induce apoptosis [Hughes et al 1999, Prog Neurobiol 57:421-50], and so the regulation of its receptor by hypoxia has clear clinical significance. Hypoxia is frequently found in human tumours where macrophage infiltrates are also found. In a series of 5 patients with either ovarian or breast cancer, Activin A receptor, type I is down-regulated in the malignant tissue as compared to adjacent normal tissue in at least one patient.

Clone p1C4 represents FGF receptor activating protein 1. The protein sequence encoded by FGF receptor activating protein 1 is represented in the public databases by the accession NP_{—}055304 and is described in this patent by SEQ ID NO: 363. The nucleotide sequence is represented in the public sequence databases by the accession NM_{—}014489 and is described in this patent by SEQ ID NO: 364. Hypoxia is an important feature of several diseases, and genes that respond to this stimulus are therefore implicated in the pathogenesis and have utility in the design of therapeutic, prognostic and diagnostic products. FGF has been shown to enhance survival of cardiac cells after ischaemic insult [Sheikh et al 2001, Am J Physiol Heart Circ Physiol 280:H1039-50], and so our observation of the hypoxia-regulation of the FGF receptor activating protein 1 is highly significant for the diagnosis and treatment of ischaemic disease. FGF receptor activating protein 1 is induced in macrophages treated with the inhibitory cytokine IL-10. Hypoxia is frequently found in human tumours where macrophage infiltrates are also found. In a series of 5 patients with either ovarian or breast cancer, FGF receptor activating protein 1 is up-regulated in the malignant tissue as compared to adjacent normal tissue in at least one patient.

Clone p1C5 represents Galectin 8. The protein sequence encoded by Galectin 8 is represented in the public databases by the accession NP_{—}006490 and is described in this patent by SEQ ID NO: 365. The nucleotide sequence is represented in the public sequence databases by the accession NM_{—}006499 and is described in this patent by SEQ ID NO: 366. Hypoxia is an important feature of several diseases, and genes that respond to this stimulus are therefore implicated in the pathogenesis and have utility in the design of therapeutic, prognostic and diagnostic products. Galectin 8 is an important tumour marker [for review see Bidon et al 2001, Int J Mol Med 8:245-50], and so its hypoxia-regulation is highly significant clinically.

Clone p1C6 represents Glucose phosphate isomerase. The protein sequence encoded by Glucose phosphate isomerase is represented in the public databases by the accession NP_{—}000166 and is described in this patent by SEQ ID NO: 367. The nucleotide sequence is represented in the public sequence databases by the accession NM_{—}000175 and is described in this patent by SEQ ID NO: 368. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Glucose phosphate isomerase is induced in macrophages activated by IL-17 and also induced in macrophages activated by IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Glucose phosphate isomerase is induced in macrophages activated by TNFalpha. Hypoxia is frequently found in human tumours where macrophage infiltrates are also found. In a series of 5 patients with either ovarian or breast cancer, Glucose phosphate isomerase is up-regulated in the malignant tissue as compared to adjacent normal tissue in at least one patient.

Clone p1C7 represents D123. The protein sequence encoded by D123 is represented in the public databases by the accession NP_{—}006014 and is described in this patent by SEQ ID NO: 369. The nucleotide sequence is represented in the public sequence databases by the accession NM_{—}006023 and is described in this patent by SEQ ID NO: 370. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. D123 is repressed in macrophages activated by LPS and gamma interferon and is also repressed in macrophages activated by IL-15. D123 protein is an important regulator of the cell cycle [Onisto et al 1998, Exp Cell Res 242:451-9]. Recently it has been shown to be regulated by modification and turnover [Okuda et al 2001, Cell Struct Funct 26:205-14]. We have shown the hypoxia-regulation of this protein, and also of several prolyl hydroxylases which are known to target proteins for ubiquitination and proteasomal degradation. We believe that concerted hypoxic control of D123 and its regulating prolyl hydroxylase is part of the means of hypoxic regulation of cell growth and tissue re-modelling.

Clone p1C8 represents DEC-1. The protein sequence encoded by DEC-1 is represented in the public databases by the accession NP_{—}003661 and is described in this patent by SEQ ID NO: 371. The nucleotide sequence is represented in the public sequence databases by the accession NM_{—}003670 and is described in this patent by SEQ ID NO: 372. Hypoxia is an important feature of several diseases, and genes that respond to this stimulus are therefore implicated in the pathogenesis and have utility in the design of therapeutic, prognostic and diagnostic products. DEC-1 is a helix-loop-helix transcription factor, and its hypoxia-regulation is highly significant. The response of renal epithelial cells to hypoxia is pertinent to kidney failure, especially regarding the medullary tissue. DEC-1 is preferentially induced by hypoxia in renal epithelial cells. Hypoxia is frequently found in human tumours where macrophage infiltrates are also found. In a series of 5 patients with either ovarian or breast cancer, DEC-1 is up-regulated and also down-regulated in the malignant tissue as compared to adjacent normal tissue in at least one patient.

Clone p1C9 represents RAB-8b protein. The protein sequence encoded by RAB-8b protein is represented in the public databases by the accession NP_{—}057614 and is described in this patent by SEQ ID NO: 373. The nucleotide sequence is represented in the public sequence databases by the accession NM_{—}016530 and is described in this patent by SEQ ID NO: 374. Hypoxia is an important feature of several diseases, and genes that respond to this stimulus are therefore implicated in the pathogenesis and have utility in the design of therapeutic, prognostic and diagnostic products. The hypoxia regulation of this small GTP-ase, which is involved in intracellular membrane trafficking, is highly significant. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. RAB-8b protein is induced in macrophages activated by LPS and gamma interferon.

Clone p1C10 represents Regulator of G-protein signalling 1. The protein sequence encoded by Regulator of G-protein signalling 1 is represented in the public databases by the accession NP_{—}002913 and is described in this patent by SEQ ID NO: 375. The nucleotide sequence is represented in the public sequence databases by the accession NM_{—}002922 and is described in this patent by SEQ ID NO: 376. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Regulator of G-protein signalling 1 is preferentially induced by hypoxia in monocytes or macrophages. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Regulator of G-protein signalling 1 is repressed in macrophages activated by LPS and gamma interferon. Hypoxia is frequently found in human tumours where macrophage infiltrates are also found. In a series of 5 patients with either ovarian or breast cancer, Regulator of G-protein signalling 1 is down-regulated in the malignant tissue as compared to adjacent normal tissue in at least one patient.

Clone p1C11 represents Polyubiquitin. The protein sequence encoded by Polyubiquitin is represented in the public databases by the accession BAA23632 and is described in this patent by SEQ ID NO: 377. The nucleotide sequence is represented in the public sequence databases by the accession AB009010 and is described in this patent by SEQ ID NO: 378. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Polyubiquitin is repressed in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Polyubiquitin is induced in macrophages activated by TNFalpha. Hypoxia is frequently found in human tumours where macrophage infiltrates are also found. In a series of 5 patients with either ovarian or breast cancer, Polyubiquitin is up-regulated in the malignant tissue as compared to adjacent normal tissue in at least one patient.

Clone p1C12 represents Integrin, alpha 5. The protein sequence encoded by Integrin, alpha 5 is represented in the public databases by the accession NP_{—}002196 and is described in this patent by SEQ ID NO: 379. The nucleotide sequence is represented in the public sequence databases by the accession NM_{—}002205 and is described in this patent by SEQ ID NO: 380. Hypoxia is an important feature of several diseases, and genes that respond to this stimulus are therefore implicated in the pathogenesis and have utility in the design of therapeutic, prognostic and diagnostic products. Integrin, alpha 5 may play a role in the response to neuronal injury [King et al 2001, J Neurocytol 30:243-52]. Our observation of hypoxia regulation of both COX-2 and integrin, alpha 5 supports the very recent suggestion that they may both function in recovery from cardiovascular injury [Hein et al 2001, Am J Physiol Heart Circ Physiol 281:H2378-84], which is pre-figured by our claims. Integrin, alpha 5 is induced by hypoxia in mammary epithelial cells, and may play an important role in cancer progression in that tissue through its function of regulating interaction with the extracellular matrix.

Clone p1C13 represents Jk-recombination signal binding protein. The protein sequence encoded by Jk-recombination signal binding protein is represented in the public databases by the accession AAA60258 and is described in this patent by SEQ ID NO: 381. The nucleotide sequence is represented in the public sequence databases by the accession L07872 and is described in this patent by SEQ ID NO: 382. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Jk-recombination signal binding protein is repressed in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Jk-recombination signal binding protein is induced in macrophages activated by TNFalpha. The important role of Jk-recombination signal binding protein in the regulation of the immune response is thus modulated by hypoxia, and there are potentially many ways of exploiting that modulation in the design of diagnostics and therapeutics. Hypoxia is frequently found in human tumours where macrophage infiltrates are also found. In a series of 5 patients with either ovarian or breast cancer, Jk-recombination signal binding protein is down-regulated in the malignant tissue as compared to adjacent normal tissue in at least one patient. It is of particular interest and significance, in view of the escape from immunological surveillance of many tumours, that Jk-recombination signal binding protein is down-regulated.

Clone p1C14 represents Abstrakt. The protein sequence encoded by Abstrakt is represented in the public databases by the accession NP_{—}057306 and is described in this patent by SEQ ID NO: 383. The nucleotide sequence is represented in the public sequence databases by the accession NM_{—}016222 and is described in this patent by SEQ ID NO: 384. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Abstrakt is repressed in macrophages activated by IL-15. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Abstrakt is induced in macrophages activated by TNFalpha. The general role of Abstrakt in the regulation of gene expression [Schmucker et al 2000, Mech Dev 91:189-96] implies particular significance to the recovery of cells from hypoxic insult.

Clone p1C15 represents High-mobility group protein 2. The protein sequence encoded by High-mobility group protein 2 is represented in the public databases by the accession NP_{—}002120 and is described in this patent by SEQ ID NO: 385. The nucleotide sequence is represented in the public sequence databases by the accession NM_{—}002129 and is described in this patent by SEQ ID NO: 386. Hypoxia is an important feature of several diseases, and genes that respond to this stimulus are therefore implicated in the pathogenesis and have utility in the design of therapeutic, prognostic and diagnostic products.

Clone p1C16 represents Decidual protein induced by progesterone. The protein sequence encoded by Decidual protein induced by progesterone is represented in the public databases by the accession NP_{—}008952 and is described in this patent by SEQ ID NO: 387. The nucleotide sequence is represented in the public sequence databases by the accession NM_{—}007021 and is described in this patent by SEQ ID NO: 388. Hypoxia is an important feature of several diseases, and genes that respond to this stimulus are therefore implicated in the pathogenesis and have utility in the design of therapeutic, prognostic and diagnostic products. Decidual protein induced by progesterone is preferentially induced by hypoxia in mammary epithelial cells. Human decidual cells have not been tested, but we predict that Decidual protein induced by progesterone is hypoxia-regulated in those cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Decidual protein induced by progesterone is repressed in macrophages activated by IL-17.

Clone p1C19 represents GM2 ganglioside activator protein. The protein sequence encoded by GM2 ganglioside activator protein is represented in the public databases by the accession NP_{—}000396 and is described in this patent by SEQ ID NO: 389. The nucleotide sequence is represented in the public sequence databases by the accession NM_{—}000405 and is described in this patent by SEQ ID NO: 390. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. GM2 ganglioside activator protein is preferentially induced by hypoxia in monocytes or macrophages. The hypoxia-inducibility of this protein in macrophages is likely to be clinically very significant. It is likely to play a role in the control of inflammation in asthma and inflammatory bowel disease, and in lipid metabolism and phosphatidylinositol-mediated signalling.

Clone p1C20 represents CNOT8. The protein sequence encoded by CNOT8 is represented in the public databases by the accession NP_{—}004770 and is described in this patent by SEQ ID NO: 391. The nucleotide sequence is represented in the public sequence databases by the accession NM_{—}004779 and is described in this patent by SEQ ID NO: 392. Hypoxia is an important feature of several diseases, and genes that respond to this stimulus are therefore implicated in the pathogenesis and have utility in the design of therapeutic, prognostic and diagnostic products.

The protein sequence encoded by Similar to Nucleoside phosphorylase is not represented in the public databases by a protein accession. The nucleotide sequence is represented in the public sequence databases by the accession AA430382 and is described in this patent by SEQ ID NO: 394. Hypoxia is an important feature of several diseases, and genes that respond to this stimulus are therefore implicated in the pathogenesis and have utility in the design of therapeutic, prognostic and diagnostic products.

Clone p1P5 represents SCYA2. The protein sequence encoded by SCYA2 is represented in the public databases by the accession NP_{—}002973 and is described in this patent by SEQ ID NO: 395. The nucleotide sequence is represented in the public sequence databases by the accession NM_{—}002982 and is described in this patent by SEQ ID NO: 396. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SCYA2 is induced in macrophages activated by LPS and gamma interferon and is also induced in macrophages activated by IL-17 or IL-15. Thus the role of SCYA2 in monocyte recruitment [Lu et al 1998, J Exp Med 187:601-8], which has clear relevance to the diagnosis and treatment of cardiovascular disease, cancer, rheumatoid arthritis, atherosclerosis and COPD, is enhanced by hypoxia. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. SCYA2 is repressed in macrophages activated by TNFalpha. Hypoxia is frequently found in human tumours where macrophage infiltrates are also found. In a series of 5 patients with either ovarian or breast cancer, SYCA2 is down-regulated in the malignant tissue as compared to adjacent normal tissue in at least one patient.

Clone p2L23 represents Endothelin 1. The protein sequence encoded by Endothelin 1 is represented in the public databases by the accession NP_{—}001946 and is described in this patent by SEQ ID NO: 397. The nucleotide sequence is represented in the public sequence databases by the accession NM_{—}001955 and is described in this patent by SEQ ID NO: 398. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Endothelin 1 is induced in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. Endothelin 1 is induced in macrophages activated by TNFalpha. Endothelin 1 plays an important role in inducing proliferation of vascular smooth muscle cells. Its hypoxia-inducibility and thus its modulation to ameliorate the consequences of ischaemic insult, is of considerable clinical significance to the recovery from injury, and angiogenesis.

The protein sequence encoded by Similar to Heat shock 70 kD protein 4 is not represented in the public databases by a protein accession. The nucleotide sequence is represented in the public sequence databases by the accession AA633656 and is described in this patent by SEQ ID NO: 400. Hypoxia is an important feature of several diseases, and genes that respond to this stimulus are therefore implicated in the pathogenesis and have utility in the design of therapeutic, prognostic and diagnostic products.

Clone p1K9 represents Lipocortin I. The protein sequence encoded by Lipocortin I is represented in the public databases by the accession NP_{—}000691 and is described in this patent by SEQ ID NO: 401. The nucleotide sequence is represented in the public sequence databases by the accession NM_{—}000700 and is described in this patent by SEQ ID NO: 402. Hypoxia is an important feature of several diseases, and genes that respond to this stimulus are therefore implicated in the pathogenesis and have utility in the design of therapeutic, prognostic and diagnostic products. Lipocortin I (also called annexin I) is an important anti-inflammatory mediator, and its hypoxia-inducibility has important implications for the diagnosis and treatment of ischaemic disease, cancer, atherosclerosis, and inflammatory diseases such as rheumatoid arthritis. Hypoxia is frequently found in human tumours where macrophage infiltrates are also found. In a series of 5 patients with either ovarian or breast cancer, Lipocortin I is up-regulated in the malignant tissue as compared to adjacent normal tissue in at least one patient. Hypoxia is frequently found in human tumours where macrophage infiltrates are also found. In a series of 5 patients with either ovarian or breast cancer, Lipocortin I is down-regulated in the malignant tissue as compared to adjacent normal tissue in at least one patient.

Clone p1K23 represents MYC. The protein sequence encoded by MYC is represented in the public databases by the accession NP_{—}002458 and is described in this patent by SEQ ID NO: 403. The nucleotide sequence is represented in the public sequence databases by the accession NM_{—}002467 and is described in this patent by SEQ ID NO: 404. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. MYC is repressed in macrophages activated by LPS and gamma interferon. Hypoxia is frequently found in human tumours where macrophage infiltrates are also found. In a series of 5 patients with either ovarian or breast cancer, MYC is up-regulated and also down-regulated in the malignant tissue as compared to adjacent normal tissue in at least one patient.

Clone p1K15 represents Alpha-2-macroglobulin. The protein sequence encoded by Alpha-2-macroglobulin is represented in the public databases by the accession NP_{—}000005 and is described in this patent by SEQ ID NO: 405. The nucleotide sequence is represented in the public sequence databases by the accession NM_{—}000014 and is described in this patent by SEQ ID NO: 406. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Alpha-2-macroglobulin is preferentially induced by hypoxia in monocytes or macrophages. Hypoxia is frequently found in human tumours where macrophage infiltrates are also found. In a series of 5 patients with either ovarian or breast cancer, Alpha-2-macroglobulin is down-regulated in the malignant tissue as compared to adjacent normal tissue in at least one patient.

Clone p1K8 represents SCYA4. The protein sequence encoded by SCYA4 is represented in the public databases by the accession XP_{—}008449 and is described in this patent by SEQ ID NO: 407. The nucleotide sequence is represented in the public sequence databases by the accession XM_{—}008449 and is described in this patent by SEQ ID NO: 408. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SCYA4 is induced in macrophages activated by LPS and gamma interferon and is also induced in macrophages activated by IL-17. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. SCYA4 is induced in macrophages activated by TNFalpha. SCYA4 is a chemokine which is likely to be significant in inflammatory disease as a direct result of its hypoxic regulation. Hypoxia is frequently found in human tumours where macrophage infiltrates are also found. In a series of 5 patients with either ovarian or breast cancer, SCYA4 is down-regulated in the malignant tissue as compared to adjacent normal tissue in at least one patient.

Clone p1M24 represents Sex hormone-binding globulin. The protein sequence encoded by Sex hormone-binding globulin is represented in the public databases by the accession NP_{—}001031 and is described in this patent by SEQ ID NO: 409. The nucleotide sequence is represented in the public sequence databases by the accession NM_{—}001040 and is described in this patent by SEQ ID NO: 410. Hypoxia is an important feature of several diseases, and genes that respond to this stimulus are therefore implicated in the pathogenesis and have utility in the design of therapeutic, prognostic and diagnostic products.

Clone p1K7 represents ATP-binding cassette E1. The protein sequence encoded by ATP-binding cassette E1 is represented in the public databases by the accession NP_{—}002931 and is described in this patent by SEQ ID NO: 411. The nucleotide sequence is represented in the public sequence databases by the accession NM_{—}002940 and is described in this patent by SEQ ID NO: 412. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. ATP-binding cassette E1 is repressed in macrophages activated by LPS and gamma interferon.

Clone p1K16 represents CCT6A. The protein sequence encoded by CCT6A is represented in the public databases by the accession NP_{—}001753 and is described in this patent by SEQ ID NO: 413. The nucleotide sequence is represented in the public sequence databases by the accession NM_{—}001762 and is described in this patent by SEQ ID NO: 414. Hypoxia is an important feature of several diseases, and genes that respond to this stimulus are therefore implicated in the pathogenesis and have utility in the design of therapeutic, prognostic and diagnostic products.

Clone p1K18 represents Colony-stimulating factor1. The protein sequence encoded by Colony-stimulating factor1 is represented in the public databases by the accession AAA52117 and is described in this patent by SEQ ID NO: 415. The nucleotide sequence is represented in the public sequence databases by the accession M37435 and is described in this patent by SEQ ID NO: 416. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Colony-stimulating factorl is repressed in macrophages activated by LPS and gamma interferon.

Clone p1N1 represents GA17. The protein sequence encoded by GA17 is represented in the public databases by the accession NP_{—}006351 and is described in this patent by SEQ ID NO: 417. The nucleotide sequence is represented in the public sequence databases by the accession NM_{—}006360 and is described in this patent by SEQ ID NO: 418. Hypoxia is an important feature of several diseases, and genes that respond to this stimulus are therefore implicated in the pathogenesis and have utility in the design of therapeutic, prognostic and diagnostic products.

Clone p1K22 represents GPR44. The protein sequence encoded by GPR44 is represented in the public databases by the accession NP_{—}004769 and is described in this patent by SEQ ID NO: 419. The nucleotide sequence is represented in the public sequence databases by the accession NM_{—}004778 and is described in this patent by SEQ ID NO: 420. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. GPR44 is repressed in macrophages activated by LPS and gamma interferon. GPR44 is most similar to the chemoattractant GPCR's [Marchese et al 1999, Genomics Feb. 15, 1999;56(1):12-21]. Our demonstration of its hypoxic regulation enables prediction of roles in diseases associated with transient hypoxia and macrophages. GPCR's are a druggable class of molecules, and represent an ideal route for pharmacological intervention.

Clone p1K14 represents Keratin 6B. The protein sequence encoded by Keratin 6B is represented in the public databases by the accession NP_{—}005546 and is described in this patent by SEQ ID NO: 421. The nucleotide sequence is represented in the public sequence databases by the accession NM_{—}005555 and is described in this patent by SEQ ID NO: 422. Hypoxia is an important feature of several diseases, and genes that respond to this stimulus are therefore implicated in the pathogenesis and have utility in the design of therapeutic, prognostic and diagnostic products. Keratin 6B is induced in macrophages treated with the inhibitory cytokine IL-10. Keratin 6B is repressed in macrophages activated by IL-17 and is also repressed in macrophages activated by IL-15.

Clone p1K13 represents Lymphocyte adaptor protein. The protein sequence encoded by Lymphocyte adaptor protein is represented in the public databases by the accession NP_{—}005466 and is described in this patent by SEQ ID NO: 423. The nucleotide sequence is represented in the public sequence databases by the accession NM_{—}005475 and is described in this patent by SEQ ID NO: 424. Hypoxia is an important feature of several diseases, and genes that respond to this stimulus are therefore implicated in the pathogenesis and have utility in the design of therapeutic, prognostic and diagnostic products.

Clone p1J20 represents Neuro-oncological ventral antigen 1. The protein sequence encoded by Neuro-oncological ventral antigen 1 is represented in the public databases by the accession NP_{—}002506 and is described in this patent by SEQ ID NO: 425. The nucleotide sequence is represented in the public sequence databases by the accession NM_{—}002515 and is described in this patent by SEQ ID NO: 426. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, Neuro-oncological ventral antigen 1 is down-regulated in the malignant tissue as compared to adjacent normal tissue in at least one patient.

Clone p1J22 represents Neutral sphingomyelinase (N-SMase) activation associated factor. The protein sequence encoded by Neutral sphingomyelinase (N-SMase) activation associated factor is represented in the public databases by the accession NP_{—}003571 and is described in this patent by SEQ ID NO: 427. The nucleotide sequence is represented in the public sequence databases by the accession NM_{—}003580 and is described in this patent by SEQ ID NO: 428. Hypoxia is an important feature of several diseases, and genes that respond to this stimulus are therefore implicated in the pathogenesis and have utility in the design of therapeutic, prognostic and diagnostic products. Neutral sphingomyelinase (N-SMase) activation associated factor is induced in macrophages treated with the inhibitory cytokine IL-10. Neutral sphingomyelinase (N-SMase) activation associated factor is repressed in macrophages activated by IL-17 and is also repressed in macrophages activated by IL-15. We expect activation of of Neutral sphingomyelinase (N-SMase) to have an anti-inflammatory effect. This enzyme is known to modulate the sphingomyelin second messenger cycle, potentially interacting with the oxidative system. Our demonstration of hypoxic regulation provides a crucial indication of the benefit of therapeutic intervention via sphingomyelinase (N-SMase) for the treatment of inflammatory diseases and diseases related to the hypoxic macrophage.

Clone p1K1 represents Cyclophilin F. The protein sequence encoded by Cyclophilin F is represented in the public databases by the accession NP_{—}005720 and is described in this patent by SEQ ID NO: 429. The nucleotide sequence is represented in the public sequence databases by the accession NM_{—}005729 and is described in this patent by SEQ ID NO: 430. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, Cyclophilin F is up-regulated in the malignant tissue as compared to adjacent normal tissue in at least one patient.

Clone p1K3 represents Pleckstrin. The protein sequence encoded by Pleckstrin is represented in the public databases by the accession NP_{—}002655 and is described in this patent by SEQ ID NO: 431. The nucleotide sequence is represented in the public sequence databases by the accession NM_{—}002664 and is described in this patent by SEQ ID NO: 432. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Pleckstrin is preferentially induced by hypoxia in monocytes or macrophages. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Pleckstrin is induced in macrophages activated by LPS and gamma interferon. Hypoxia is frequently found in human tumours where macrophage infiltrates are also found. In a series of 5 patients with either ovarian or breast cancer, Pleckstrin is down-regulated in the malignant tissue as compared to adjacent normal tissue in at least one patient.

Clones p1J19 and p1K2 represent CFFM4. The protein sequence encoded by CFFM4 is represented in the public databases by the accession NP_{—}067024 and is described in this patent by SEQ ID NO: 433. The nucleotide sequence is represented in the public sequence databases by the accession NM_{—}021201 and is described in this patent by SEQ ID NO: 434. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. CFFM4 is preferentially induced by hypoxia in monocytes or macrophages. CFFM4 is induced in macrophages treated with the inhibitory cytokine IL-10. It has been suggested recently that CFFM4 is associated with mature cellular function in the monocytic lineage and that it may be a component of a receptor complex involved in signal transduction [Gingras et al 2001, Immunogenetics 53:468-76]. Our demonstration of hypoxic-regulation opens possible routes of intervention in macrophage-related disease via this potentially important cell surface receptor. Hypoxia is frequently found in human tumours where macrophage infiltrates are also found. In a series of 5 patients with either ovarian or breast cancer, CFFM4 is down-regulated in the malignant tissue as compared to adjacent normal tissue in at least one patient.

Clone p1K5 represents Ribosomal protein L36a. The protein sequence encoded by Ribosomal protein L36a is represented in the public databases by the accession NP_{—}000992 and is described in this patent by SEQ ID NO: 435. The nucleotide sequence is represented in the public sequence databases by the accession NM_{—}001001 and is described in this patent by SEQ ID NO: 436. Hypoxia is an important feature of several diseases, and genes that respond to this stimulus are therefore implicated in the pathogenesis and have utility in the design of therapeutic, prognostic and diagnostic products.

Clone p1J17 represents SLC6A1. The protein sequence encoded by SLC6A1 is represented in the public databases by the accession NP_{—}003033 and is described in this patent by SEQ ID NO: 437. The nucleotide sequence is represented in the public sequence databases by the accession NM_{—}003042 and is described in this patent by SEQ ID NO: 438. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, SLC6A1 is up-regulated and also down-regulated in the malignant tissue as compared to adjacent normal tissue in at least one patient.

Clone p1J18 represents Synaptopodin. The protein sequence encoded by Synaptopodin is represented in the public databases by the accession NP_{—}009217 and is described in this patent by SEQ ID NO: 439. The nucleotide sequence is represented in the public sequence databases by the accession NM_{—}007286 and is described in this patent by SEQ ID NO: 440. Hypoxia is an important feature of several diseases, and genes that respond to this stimulus are therefore implicated in the pathogenesis and have utility in the design of therapeutic, prognostic and diagnostic products. Synaptopodin is a component of the cytoskeleton which has particular importance in neurons, where it is involved in synaptic plasticity. Its hypoxia-regulation is clearly potentially significant in the context of neurological disease. Hypoxia is frequently found in human tumours where macrophage infiltrates are also found. In a series of 5 patients with either ovarian or breast cancer, Synaptopodin is up-regulated and also down-regulated in the malignant tissue as compared to adjacent normal tissue in at least one patient.

Clone p1J15 represents TERA protein. The protein sequence encoded by TERA protein is represented in the public databases by the accession NP_{—}067061 and is described in this patent by SEQ ID NO: 441. The nucleotide sequence is represented in the public sequence databases by the accession NM_{—}021238 and is described in this patent by SEQ ID NO: 442. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, TERA protein is up-regulated and also down-regulated in the malignant tissue as compared to adjacent normal tissue in at least one patient.

Clone p1K4 represents TSC-22. The protein sequence encoded by TSC-22 is represented in the public databases by the accession NP_{—}006013 and is described in this patent by SEQ ID NO: 443. The nucleotide sequence is represented in the public sequence databases by the accession NM_{—}006022 and is described in this patent by SEQ ID NO: 444. Hypoxia is an important feature of several diseases, and genes that respond to this stimulus are therefore implicated in the pathogenesis and have utility in the design of therapeutic, prognostic and diagnostic products. TSC-22 is a transcriptional regulator of the leucine zipper class, and its hypoxic regulation is likely to have significant downstream effects which may be related to ischaemic disease. Thus it may provide important points of intervention in such diseases. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. TSC-22 is repressed in macrophages activated by LPS and gamma interferon. Hypoxia is frequently found in human tumours where macrophage infiltrates are also found. In a series of 5 patients with either ovarian or breast cancer, TSC-22 is down-regulated in the malignant tissue as compared to adjacent normal tissue in at least one patient.

Clone p2A14 represents an unannotated EST. The protein sequence encoded by this EST is not represented in the public databases by a protein accession. The nucleotide sequence is represented in the public sequence databases by the accession AA988110 and is described in this patent by SEQ ID NO: 446. Hypoxia is an important feature of several diseases, and genes that respond to this stimulus are therefore implicated in the pathogenesis and have utility in the design of therapeutic, prognostic and diagnostic products. The EST represented by SEQ ID NO: 446 is induced in macrophages treated with the inhibitory cytokine IL-10. The EST represented by SEQ ID NO: 446 is repressed in macrophages activated by IL-17 and is also repressed in macrophages activated by IL-15.

Clone p1J23 represents Calgranulin A. The protein sequence encoded by Calgranulin A is represented in the public databases by the accession NP_{—}002955 and is described in this patent by SEQ ID NO: 447. The nucleotide sequence is represented in the public sequence databases by the accession NM_{—}002964 and is described in this patent by SEQ ID NO: 448. Hypoxia is an important feature of several diseases, and genes that respond to this stimulus are therefore implicated in the pathogenesis and have utility in the design of therapeutic, prognostic and diagnostic products. Calgranulin A, called by its synonym S100A8, has been cited recently as “wound-regulated” [Thorey et al 2001, J Biol Chem 276:35818-25] which provides less precise support for our prior determination of its hypoxia-regulation. In its potential role as a chemoattractant, it would be an important point of intervention for the modulation of inflamnmatory processes. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Calgranulin A is repressed in macrophages activated by LPS and gamma interferon. Hypoxia is frequently found in human tumours where macrophage infiltrates are also found. In a series of 5 patients with either ovarian or breast cancer, Calgranulin A is down-regulated in the malignant tissue as compared to adjacent normal tissue in at least one patient.

Clone p1J21 represents Replication factor C large subunit. The protein sequence encoded by Replication factor C large subunit is represented in the public databases by the accession NP_{—}002904 and is described in this patent by SEQ ID NO: 449. The nucleotide sequence is represented in the public sequence databases by the accession NM_{—}002913 and is described in this patent by SEQ ID NO: 450. Hypoxia is an important feature of several diseases, and genes that respond to this stimulus are therefore implicated in the pathogenesis and have utility in the design of therapeutic, prognostic and diagnostic products.

Clone p1J24 represents Signal recognition particle 19 kD. The protein sequence encoded by Signal recognition particle 19 kD is represented in the public databases by the accession NP_{—}003126 and is described in this patent by SEQ ID NO: 451. The nucleotide sequence is represented in the public sequence databases by the accession NM_{—}003135 and is described in this patent by SEQ ID NO: 452. Hypoxia is an important feature of several diseases, and genes that respond to this stimulus are therefore implicated in the pathogenesis and have utility in the design of therapeutic, prognostic and diagnostic products.

Clone p1J16 represents cDNA: FLJ23019 fis, clone LNG00916. The protein sequence encoded by cDNA: FLJ23019 fis, clone LNG00916 is not represented in the public databases by a protein accession. The nucleotide sequence is represented in the public sequence databases by the accession AK026672 and is described in this patent by SEQ ID NO: 454. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. The cDNA: FLJ23019 fis, clone LNG00916 is induced in macrophages activated by LPS and gamma interferon and is also induced in macrophages activated by IL-15.

Clone p1J2 represents Proteasome subunit, alpha type, 4. The protein sequence encoded by Proteasome subunit, alpha type, 4 is represented in the public databases by the accession NP_{—}002780 and is described in this patent by SEQ ID NO: 455. The nucleotide sequence is represented in the public sequence databases by the accession NM_{—}002789 and is described in this patent by SEQ ID NO: 456. Hypoxia is an important feature of several diseases, and genes that respond to this stimulus are therefore implicated in the pathogenesis and have utility in the design of therapeutic, prognostic and diagnostic products.

Clone p1J9 represents MAFB. The protein sequence encoded by MAFB is represented in the public databases by the accession NP_{—}005452 and is described in this patent by SEQ ID NO: 457. The nucleotide sequence is represented in the public sequence databases by the accession NM_{—}005461 and is described in this patent by SEQ ID NO: 458. Hypoxia is an important feature of several diseases, and genes that respond to this stimulus are therefore implicated in the pathogenesis and have utility in the design of therapeutic, prognostic and diagnostic products. MAFB is a transcriptional regulator of the leucine zipper type, and is likely to play an important role in the mediation of the hypoxic response, with attendant relevance to associated diseases.

Clone p1J10 represents DNCLI2. The protein sequence encoded by DNCLI2 is represented in the public databases by the accession NP_{—}006132 and is described in this patent by SEQ ID NO: 459. The nucleotide sequence is represented in the public sequence databases by the accession NM_{—}006141 and is described in this patent by SEQ ID NO: 460. Hypoxia is an important feature of several diseases, and genes that respond to this stimulus are therefore implicated in the pathogenesis and have utility in the design of therapeutic, prognostic and diagnostic products. Hypoxia is frequently found in human tumours where macrophage infiltrates are also found. In a series of 5 patients with either ovarian or breast cancer, gene X is down-regulated in the malignant tissue as compared to adjacent normal tissue in at least one patient.

Clone p1J1 represents Chromobox homolog 3. The protein sequence encoded by Chromobox homolog 3 is represented in the public databases by the accession NP_{—}057671 and is described in this patent by SEQ ID NO: 461. The nucleotide sequence is represented in the public sequence databases by the accession NM_{—}016587 and is described in this patent by SEQ ID NO: 462. Hypoxia is an important feature of several diseases, and genes that respond to this stimulus are therefore implicated in the pathogenesis and have utility in the design of therapeutic, prognostic and diagnostic products.

Clone p1J5 represents SCYA7. The protein sequence encoded by SCYA7 is represented in the public databases by the accession NP_{—}006264 and is described in this patent by SEQ ID NO: 463. The nucleotide sequence is represented in the public sequence databases by the accession NM_{—}006273 and is described in this patent by SEQ ID NO: 464. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SCYA7 is induced in macrophages activated by IL-15. SCYA7 is a chemoattractant protein which, considering its hypoxia-regulation, is likely to play an important role in inflammatory and ischaemic disease. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. SCYA7 is repressed in macrophages activated by TNFalpha.

Clone p1J11 represents Fatty-acid-Coenzyme A ligase, long-chain 2. The protein sequence encoded by Fatty-acid-Coenzyme A ligase, long-chain 2 is represented in the public databases by the accession NP_{—}066945 and is described in this patent by SEQ ID NO: 465. The nucleotide sequence is represented in the public sequence databases by the accession NM_{—}021122 and is described in this patent by SEQ ID NO: 466. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Fatty-acid-Coenzyme A ligase, long-chain 2 is induced in macrophages activated by LPS and gamma interferon and also induced in macrophages activated by IL-17 or IL-15. Hypoxia is frequently found in human tumours where macrophage infiltrates are also found. In a series of 5 patients with either ovarian or breast cancer, Fatty-acid-Coenzyme A ligase, long-chain 2 is down-regulated in the malignant tissue as compared to adjacent normal tissue in at least one patient.

Clone p1J8 represents Programmed cell death 5. The protein sequence encoded by Programmed cell death 5 is represented in the public databases by the accession NP_{—}004699 and is described in this patent by SEQ ID NO: 467. The nucleotide sequence is represented in the public sequence databases by the accession NM_{—}004708 and is described in this patent by SEQ ID NO: 468. Hypoxia is an important feature of several diseases, and genes that respond to this stimulus are therefore implicated in the pathogenesis and have utility in the design of therapeutic, prognostic and diagnostic products.

Clone p1I20 represents SCYA3L. The protein sequence encoded by SCYA3L is represented in the public databases by the accession CAA36397 and is described in this patent by SEQ ID NO: 469. The nucleotide sequence is represented in the public sequence databases by the accession X52149 and is described in this patent by SEQ ID NO: 470. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. SCYA3L is preferentially induced by hypoxia in monocytes or macrophages. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SCYA3L is induced in macrophages activated by LPS and gamma interferon. TNFalpha is an inflammatory cytokine, which acts on macrophages, and has been shown to be central to the pathophysiology and treatment of diseases including rheumatoid arthritis. Genes that change in expression in response to TNFalpha therefore have utility in the design of therapeutic, prognostic and diagnostic products for such inflammatory conditions. SCYA3L is induced in macrophages activated by TNFalpha.

Clone p1J3 represents Furin. The protein sequence encoded by Furin is represented in the public databases by the accession NP_{—}002560 and is described in this patent by SEQ ID NO: 471. The nucleotide sequence is represented in the public sequence databases by the accession NM_{—}002569 and is described in this patent by SEQ ID NO: 472. Hypoxia is an important feature of several diseases, and genes that respond to this stimulus are therefore implicated in the pathogenesis and have utility in the design of therapeutic, prognostic and diagnostic products.

Clone p1J12 represents Nuclear autoantigenic sperm protein. The protein sequence encoded by Nuclear autoantigenic sperm protein is represented in the public databases by the accession NP_{—}002473 and is described in this patent by SEQ ID NO: 473. The nucleotide sequence is represented in the public sequence databases by the accession NM_{—}002482 and is described in this patent by SEQ ID NO: 474. Hypoxia is an important feature of several diseases, and genes that respond to this stimulus are therefore implicated in the pathogenesis and have utility in the design of therapeutic, prognostic and diagnostic products.

Clone p1I23 represents Ecotropic viral integration site 2A. The protein sequence encoded by Ecotropic viral integration site 2A is represented in the public databases by the accession NP_{—}055025 and is described in this patent by SEQ ID NO: 475. The nucleotide sequence is represented in the public sequence databases by the accession NM_{—}014210 and is described in this patent by SEQ ID NO: 476. Hypoxia is an important feature of several diseases, and genes that respond to this stimulus are therefore implicated in the pathogenesis and have utility in the design of therapeutic, prognostic and diagnostic products. There is a prejudice in the art that the response to hypoxia is generic to all cell types. Contrary to this, we show that genes are regulated by hypoxia to a greater degree in certain cell types, substantiating their utility in designing specific therapeutic products for diseases involving those cell types. Monocytes and macrophages have been implicated in the following diseases involving hypoxia: rheumatoid arthritis, atherosclerosis, cancer, COPD. Ecotropic viral integration site 2A is preferentially induced by hypoxia in monocytes or macrophages. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Ecotropic viral integration site 2A is repressed in macrophages activated by LPS and gamma interferon. Hypoxia is frequently found in human tumours where macrophage infiltrates are also found. In a series of 5 patients with either ovarian or breast cancer, Ecotropic viral integration site 2A is down-regulated in the malignant tissue as compared to adjacent normal tissue in at least one patient.

Clone p1J7 represents Sjogren syndrome antigen B. The protein sequence encoded by Sjogren syndrome antigen B is represented in the public databases by the accession NP_{—}003133 and is described in this patent by SEQ ID NO: 477. The nucleotide sequence is represented in the public sequence databases by the accession NM_{—}003142 and is described in this patent by SEQ ID NO: 478. Hypoxia is an important feature of several diseases, and genes that respond to this stimulus are therefore implicated in the pathogenesis and have utility in the design of therapeutic, prognostic and diagnostic products. Sjogren syndrome antigen B is preferentially induced by hypoxia in mammary epithelial cells. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. Sjogren syndrome antigen B is induced in macrophages activated by LPS and gamma interferon.

Clone p1I21 represents SCYA8. The protein sequence encoded by SCYA8 is represented in the public databases by the accession NP_{—}005614 and is described in this patent by SEQ ID NO: 479. The nucleotide sequence is represented in the public sequence databases by the accession NM_{—}005623 and is described in this patent by SEQ ID NO: 480. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. SCYA8 is induced in macrophages activated by LPS and gamma interferon and is also induced in macrophages activated by IL-15.

Clone p1I19 represents GRO2. The protein sequence encoded by GRO2 is represented in the public databases by the accession NP_{—}002080 and is described in this patent by SEQ ID NO: 481. The nucleotide sequence is represented in the public sequence databases by the accession NM_{—}002089 and is described in this patent by SEQ ID NO: 482. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. GRO2 is induced in macrophages activated by LPS and gamma interferon and is also induced in macrophages activated by IL-17. GRO2 encodes a chemokine which is likely to be involved in the inflammatory response. Its induction by hypoxia provides a potential route for intervention in diseases related to inflammation and ischaemia. Hypoxia is frequently found in human tumours where macrophage infiltrates are also found. In a series of 5 patients with either ovarian or breast cancer, GRO2 is down-regulated in the malignant tissue as compared to adjacent normal tissue in at least one patient.

Clone p1J4 represents Small nuclear ribonucleoprotein D1. The protein sequence encoded by Small nuclear ribonucleoprotein D1 is represented in the public databases by the accession NP_{—}008869 and is described in this patent by SEQ ID NO: 483. The nucleotide sequence is represented in the public sequence databases by the accession NM_{—}006938 and is described in this patent by SEQ ID NO: 484. Hypoxia is an important feature of several diseases, and genes that respond to this stimulus are therefore implicated in the pathogenesis and have utility in the design of therapeutic, prognostic and diagnostic products.

Clone p1I24 represents GRO1. The protein sequence encoded by GRO1 is represented in the public databases by the accession NP_{—}001502 and is described in this patent by SEQ ID NO: 485. The nucleotide sequence is represented in the public sequence databases by the accession NM_{—}001511 and is described in this patent by SEQ ID NO: 486. GRO1 has known chemotactic activity for neutrophils. GRO1 belongs to the intercrine alpha family of small CXC cytokines. GRO1 encodes a chemokine which is likely to be involved in the inflammatory response. Its induction by hypoxia provides a potential route for intervention in diseases related to inflammation and ischaemia. Hypoxia is an important feature of several diseases, and genes that respond to this stimulus are therefore implicated in the pathogenesis and have utility in the design of therapeutic, prognostic and diagnostic products. Macrophages are key to several diseases involving hypoxia, and contribute to inflammatory processes. In these, macrophages are frequently activated by cytokines, which have been shown to be present at disease sites, so gene expression responses to both hypoxia and cytokines are especially relevant. GRO1 is induced in macrophages activated by LPS and gamma interferon and is also induced in macrophages activated by IL-17. Hypoxia is frequently found in human tumours where macrophage infiltrates are also found. In a series of 5 patients with either ovarian or breast cancer, GRO1 is down-regulated in the malignant tissue as compared to adjacent normal tissue in at least one patient.

Clone p1I18 represents Selectin L. The protein sequence encoded by Selectin L is represented in the public databases by the accession NP_{—}000646 and is described in this patent by SEQ ID NO: 487. The nucleotide sequence is represented in the public sequence databases by the accession NM_{—}000655 and is described in this patent by SEQ ID NO: 488. Hypoxia is an important feature of several diseases, and genes that respond to this stimulus are therefore implicated in the pathogenesis and have utility in the design of therapeutic, prognostic and diagnostic products. Selectin L shedding by leucocytes is one aspect of the induction of the inflammatory response. Hypoxic-regulation of Selectin L is clearly a significant factor in the induction of inflammation following ischaemic insult or in diseases in which transient ischaemic conditions occur. Modulation of this induction is one aspect of the present invention. Hypoxia is frequently found in human tumours where macrophage infiltrates are also found. In a series of 5 patients with either ovarian or breast cancer, Selectin L is down-regulated in the malignant tissue as compared to adjacent normal tissue in at least one patient.

TABLE 1
Hypoxia-inducible genes identified from clones
only derived from the cardiomyoblast library

| | SEQ ID | | |
|---|---|---|---|
| GENE NAME | protein | nucleotide | Accession |
| Diacylglycerol kinase, zeta | 353 | 354 | NM_003646 |
| CCR4 associated factor 1 | 391 | 392 | AF053318 |
| GM2 ganglioside activator | 389 | 390 | X62078 |
| protein. | | | |
| Granulin | 269 | 270 | AK000607 |
| Serine protease 11 | 355 | 356 | Y07921 |
| High mobility group 2 protein | 385 | 386 | M83665 |
| Decidual protein induced by | 387 | 388 | NM_007021 |
| progesterone | | | |
| DEAD-box protein abstrakt | 383 | 384 | NM_016222 |
| IL-1 receptor antagonist | 357 | 358 | U65590 |
| KIAA1376 protein | 29 | 30 | AB037797 |
| hypothetical protein KIAA0127 | 31 | 32 | D50917 |
| hypothetical protein FLJ20308 | 33 | 34 | AL137263 |
| EST | 91 | 92 | AL390082 |
| EST | 89 | 90 | AL117352 |
| EST | 77 | 78 | AW664180 |

TABLE 2
Hypoxia-inducible genes identified from clones
only derived from the macrophage libraries

| | SEQID | | |
|---|---|---|---|
| GENE NAME | protein | nucleotide | Accession |
| Metallothionein-2a | 265 | 266 | J00271 |
| Metallothionein-1h | 239 | 240 | X64177 |
| Metallothionein-1G | 243 | 244 | J03910 |
| Interleukin 8 | 251 | 252 | Y00787 |
| Lactate dehydrogenase A | 223 | 224 | NM_005566 |
| UDP-glucose pyrophosphorylase 2 | 347 | 348 | NM_006759 |
| Enolase 1 | 257 | 258 | NM_001428 |
| Enolase 2 | 273 | 274 | NM_001975 |
| Tissue factor/coagulation | 225 | 226 | NM_001993 |
| factor III/thromboplastin | | | |
| proline 4-hydroxylase, alpha | 231 | 232 | NM_000917 |
| polypeptide 1 | | | |
| proline 4-hydroxylase, alpha | 349 | 350 | NM_004199 |
| polypeptide II | | | |
| NS1-binding protein | 359 | 360 | NM_006469 |
| FGF receptor activating protein | 363 | 364 | AF159621 |
| 1 | | | |
| Adenylate kinase 3 | 263 | 264 | NM_013410 |
| Osteopontin | 267 | 268 | X13694 |
| Aldolase C, fructose- | 259 | 260 | NM_005165 |
| bisphosphate | | | |
| Galectin-8 | 365 | 366 | AF193806 |
| Regulator of G-protein | 375 | 376 | S59049 |
| signalling 1 (BL34) | | | |
| Polyubiquitin UbC | 377 | 378 | AB009010 |
| Activin A receptor type I | 361 | 362 | NM_001105 |
| Glyceraldehyde-3-phosphate | 253 | 254 | NM_002046 |
| dehydrogenase | | | |
| Phosphoglycerate kinase 1 | 255 | 256 | NM_000291 |
| Rab-8b | 373 | 374 | NM_016530 |
| Glucose phosphate isomerase | 367 | 368 | NM_000175 |
| D123 gene product (HT1080) | 369 | 370 | U27112 |
| Integrin alpha 5 | 379 | 380 | NM_002205 |
| Triosephosphate isomerase 1 | 261 | 262 | NM_000365 |
| solute carrier family 31 (copper | 345 | 346 | NM_001860 |
| transporters), member 2 | | | |
| Jk-recombination signal binding | 381 | 382 | L07872 |
| protein | | | |
| N-myc downstream regulated | 229 | 230 | D87953 |
| (NDRG1/RTP) | | | |
| Plasminogen activator | 235 | 236 | M16006 |
| inhibitor-1 | | | |
| Dec-1 | 371 | 372 | NM_003670 |
| FUSIN/CXCR4 | 331 | 332 | NM_003467 |
| hypothetical protein FLJ20500 | 25 | 26 | AK000507 |
| DKFZP564D116 protein | 27 | 28 | AL050022 |
| hypothetical protein FLJ10134 | 23 | 24 | AK000996 |
| cDNA FLJ10433 fis NT2RP1000478 | 73 | 74 | AK001295 |
| ESTs | 93 | 94 | AW250104 |
| ESTs | 95 | 96 | BE382614 |
| ESTs | 67 | 68 | AW071063 |
| ESTs | 67 | 68 | AW964331 |
| ESTs | 133 | 134 | AA612751 |
| Singleton EST (not in UniGene) | 135 | 136 | AI018611 |

The gene entitled “Jk-recombination signal binding protein” was found to be hypoxia-inducible using subtracted cDNA probes for hybridization, but with non-subtracted probes, where the hybridization is quantitive, no signal was detected. This indicates that the gene is probably hypoxia-regulated but the absolute expression levels are very low.

TABLE 3
Hypoxia-inducible genes identified from clones derived
from both macrophage and myoblast libraries.

| | | | | Hypoxia/ | Hypoxia/ |
|---|---|---|---|---|---|
| | | SEQ ID | | normoxia | normoxia |
| GENE NAME | Accession | protein | nucleotide | (macrophage) | (myoblast) |
| Solute carrier family 2, | NM_006931 | 247 | 248 | 91.39 | 8.23 |
| member 3 | | | | | |
| Solute carrier family 2, | NM_003039 | 311 | 312 | 10.75 | 2.26 |
| member 5 | | | | | |
| Adiphophilin | NM_001122 | 313 | 314 | 13.97 | 5.10 |
| Hexokinase 2 | NM_000189 | 249 | 250 | 11.50 | 6.25 |
| Stearoyl-CoA desaturase | AB032261 | 351 | 352 | 3.74 | 2.31 |
| cDNA DKFZp434O071 | AF125392 | 75 | 76 | 2.31 | 2.75 |
| Hypoxia-inducible protein | NM_013332 | 271 | 272 | 3.62 | 5.07 |
| 2 | | | | | |

TABLE 4
Hypoxia responses amplified by HIF1 alpha overexpression

| | Nucl | | | | | | | | | |
|---|---|---|---|---|---|---|---|---|---|---|
| | SEQ | | | | | | | | | |
| | ID | Experimental Condition # | | | | | | | | |
| Gene Name | NO: | 1 | 2 | 3 | 4 | 5 | 6 | 7 | 8 | 9 |
| Metallothionein 2A | 265 | 1 | 0.57 | 0.69 | 3.33 | 3.22 | 5.77 | 10.37 | 2.05 | 1.70 |
| Metallothionein 1G | 244 | 1 | 0.68 | 0.64 | 4.23 | 4.21 | 7.35 | 11.03 | 3.65 | 2.28 |
| hypothetical protein hqp0376 | 338 | 1 | 0.79 | 0.61 | 6.54 | 4.44 | 9.01 | 11.54 | 4.17 | 3.22 |
| Novel Metallothionein | 84 | 1 | 0.95 | 0.78 | 5.18 | 4.36 | 8.20 | 11.16 | 3.48 | 2.94 |

Legend: Data shown in the average of 4 repeat experiments. Experimental condition is as shown in the text. Values represent fold change as compared to untreated cells (condition 1).

TABLE 5
Hypoxia responses amplified by EPASl overexpression

| | Nucl | | | | | | | | | |
|---|---|---|---|---|---|---|---|---|---|---|
| | SEQ | | | | | | | | | |
| | ID | Experimental Condition # | | | | | | | | |
| Gene Name | NO: | 1 | 2 | 3 | 4 | 5 | 6 | 7 | 8 | 9 |
| cDNA DKFZp586E1624 | 66 | 1 | 0.77 | 0.67 | 1.00 | 1.12 | 1.58 | 0.83 | 2.60 | 2.49 |
| Butyrate response factor 1 | 328 | 1 | 0.74 | 0.64 | 1.60 | 1.64 | 1.57 | 1.23 | 2.19 | 3.20 |
| hypothetical protein FLJ10134 | 24 | 1 | 0.62 | 0.53 | 2.73 | 2.09 | 2.80 | 2.87 | 4.20 | 3.65 |
| EGL nine (C. elegans) homolog 3 | 86 | 1 | 1.34 | 0.81 | 1.98 | 1.90 | 2.02 | 1.94 | 2.81 | 3.12 |
| ERO1 (S. cerevisiae)-like | 68 | 1 | 1.02 | 1.30 | 4.26 | 4.14 | 4.76 | 4.12 | 4.91 | 6.44 |
| hypothetical protein FLJ10134 | 24 | 1 | 0.68 | 0.53 | 2.03 | 1.97 | 3.01 | 2.46 | 3.67 | 2.95 |

Legend: Data shown is the average of 4 repeat experiments. Experimental condition is as shown in the text. Values represent fold change as compared to untreated cells (condition 1).

TABLE 6
Negative hypoxia responses amplified by HIF1 alpha/EPAS1 overexpression

| | Nucl | | | | | | | | | |
|---|---|---|---|---|---|---|---|---|---|---|
| | SEQ | | | | | | | | | |
| | ID | Experimental Condition # | | | | | | | | |
| Gene Name | NO: | 1 | 2 | 3 | 4 | 5 | 6 | 7 | 8 | 9 |
| hypothetical protein CGI-117 | 48 | 1 | 0.83 | 0.87 | 0.42 | 0.42 | 0.32 | 0.34 | 0.33 | 0.27 |

TABLE 7
Genes induced by hypoxia (similar response +/− cell activation)

| | | | | | | RATIO | | |
|---|---|---|---|---|---|---|---|---|
| | | IMAGE | | SEQ ID | | Hypoxia/Normoxia | | Activated/Resting |
| Row | TITLE | Id | accession | protein | nucl | (resting) | (activated) | (normoxia) |
| 1 | Activated leucocyte cell | 26617 | R13558 | 277 | 278 | 1.46 | 1.86 | 0.46 |
| | adhesion molecule | | | | | | | |
| 2 | MAX-interacting protein 1 | 435219 | AA705886 | 279 | 280 | 2.55 | 3.18 | n/d |
| 3 | BCL2/adenovirus E1B 19kD- | 814899 | AA465697 | 217 | 218 | 2.50 | 3.48 | 0.41 |
| | interacting protein 3-like | | | | | | | |
| 4 | Nuclear receptor co-repressor | 488301 | AA085748 | 281 | 282 | 1.38 | 1.75 | 0.65 |
| 5 | Enolase 2, (gamma, neuronal) | 789147 | AA450189 | 273 | 274 | 2.87 | 4.98 | 1.32 |
| 6 | Chitinase 3-like 2 | 47043 | H10721 | 283 | 284 | 1.98 | 1.98 | n/d |
| 7 | BACH1 transcription factor | 2009495 | AI336948 | 285 | 286 | 2.34 | 2.23 | 1.30 |
| 8 | Solute carrier family 2, | 453589 | AA679565 | 219 | 220 | 8.50 | 6.80 | 0.59 |
| | member 1 | | | | | | | |
| 9 | Phosphoglucomutase 1 | 843174 | AA488504 | 287 | 288 | 1.43 | 1.83 | n/d |
| 10 | PDGF beta | 67654 | T49539 | 221 | 222 | 1.66 | 1.64 | 1.09 |
| 11 | PDGF beta | 343320 | W68169 | 221 | 222 | 1.86 | 1.67 | 0.84 |
| 12 | CGI-109 protein | 144862 | R78570 | 289 | 290 | 1.42 | 1.94 | n/d |
| 13 | SAP30 | 502142 | AA126982 | 291 | 292 | 2.03 | 3.49 | n/d |
| 14 | ATP-binding cassette | 827168 | AA521292 | 293 | 294 | 2.04 | 2.24 | 1.20 |
| | transporter-1 | | | | | | | |
| 15 | SEC24 protein | 712559 | AA278134 | 295 | 296 | 2.87 | 3.97 | n/d |
| 16 | Trinucleotide repeat containing | 199367 | R95691 | 297 | 298 | 1.92 | 1.38 | 1.35 |
| | 3 | | | | | | | |
| 17 | Post-synaptic density protein | 26021 | R39954 | 299 | 300 | 1.79 | 1.64 | 1.63 |
| | 95 | | | | | | | |
| 18 | Tumor protein D52 | 814306 | AA459318 | 301 | 302 | 1.24 | 1.75 | n/d |
| 19 | Cyclin-dependent kinase | 854668 | AA630082 | 303 | 304 | 2.36 | 1.57 | 2.19 |
| | inhibitor p27kip1 | | | | | | | |
| 20 | phosphoinositide-3-kinase, | 506009 | AA708437 | 305 | 306 | 1.44 | 2.11 | 0.26 |
| | catalytic, beta | | | | | | | |
| 21 | CDNA FLJ13611 fis, clone | 49918 | H15296 | 1 | 2 | 2.33 | 2.54 | n/d |
| | PLACE1010802 | | | | | | | |
| 22 | Solute carrier family 5, | 345743 | W72666 | 307 | 308 | 3.33 | 4.42 | 1.14 |
| | member 3 | | | | | | | |
| 23 | PSCDBP | 824531 | AA490903 | 309 | 310 | 2.02 | 1.90 | n/d |
| 24 | lactate dehydrogenase A | 43550 | H05914 | 223 | 224 | 2.13 | 2.23 | 1.23 |
| 25 | Solute carrier family 2, | 190732 | H38650 | 311 | 312 | 2.72 | 3.45 | n/d |
| | member 5 | | | | | | | |
| 26 | Adipophilin | 435036 | AA700054 | 313 | 314 | 6.28 | 2.39 | n/d |
| 27 | Tissue factor | 1928791 | AI313387 | 225 | 226 | 1.34 | 2.21 | 0.62 |
| 28 | Vascular endothelial growth | 34778 | R19956 | 227 | 228 | 1.53 | 1.97 | n/d |
| | factor | | | | | | | |
| 29 | RTP/NDRG1 | 842863 | AA489261 | 229 | 230 | 3.40 | 3.06 | 2.38 |
| 30 | Early development regulator 2 | 898328 | AA598840 | 315 | 316 | 1.96 | 1.61 | 1.18 |
| 31 | Procollagen-proline 4- | 838802 | AA457671 | 231 | 232 | 2.69 | 2.32 | 1.31 |
| | hydroxylase alpha 1 | | | | | | | |
| 32 | B-cell translocation gene 1, | 298268 | N70463 | 317 | 318 | 1.91 | 2.08 | 1.84 |
| 33 | SH3PX1 | 142139 | R69163 | 319 | 320 | 1.81 | 1.15 | 1.87 |
| 34 | Cyclin G2 | 823691 | AA489752 | 321 | 322 | 1.70 | 2.47 | n/d |
| 35 | BCL2/adenovirus E1B- | 783697 | AA446839 | 233 | 234 | 4.37 | 6.52 | n/d |
| | interacting protein 3 | | | | | | | |
| 36 | BCL2/adenovirus E1B- | 359982 | AA063521 | 233 | 234 | 3.09 | 5.00 | n/d |
| | interacting protein 3 | | | | | | | |
| 37 | NAG-5 protein | 460618 | AA700447 | 323 | 324 | 1.91 | n/d | n/d |
| 38 | Cytochrome P450 IB1 (dioxin- | 782760 | AA448157 | 325 | 326 | 1.93 | 2.25 | 0.93 |
| | inducible) | | | | | | | |
| 39 | Plasminogen activator | 244307 | N75719 | 235 | 236 | 1.81 | 2.78 | n/d |
| | inhibitor, type I | | | | | | | |
| 40 | Butyrate response factor 1 | 768299 | AA424743 | 327 | 328 | 2.59 | 2.69 | 1.88 |
| 41 | Butyrate response factor 1 | 413633 | AA723035 | 327 | 328 | 2.35 | 2.36 | 1.76 |
| 42 | p8 protein (candidate of | 80484 | T64469 | 329 | 330 | 4.13 | 4.19 | n/d |
| | metastasis 1) | | | | | | | |
| 43 | Fusin/CXCR4 | 79629 | T62491 | 331 | 332 | 2.16 | 1.97 | 1.07 |
| 44 | solute carrier family 16, | 1638893 | AI016779 | 333 | 334 | 1.96 | n/d | n/d |
| | member 6 | | | | | | | |
| 45 | solute carrier family 16, | 266389 | N21654 | 333 | 334 | 2.00 | n/d | n/d |
| | member 6 | | | | | | | |
| 46 | Proline-rich protein with | 857002 | AA669637 | 335 | 336 | 2.707 | 1.80 | 7.08 |
| | nuclear targeting signal (B4-2) | | | | | | | |
| 47 | Cox-2 | 845477 | AA644211 | 237 | 238 | n/d | 8.34 | 22.38 |
| 48 | Glycogen synthase 1 (muscle) | 45632 | H08446 | 275 | 276 | n/d | 2.28 | 1.15 |
| 49 | cDNA FLJ13700 fis, clone | 261246 | H98241 | 15 | 16 | 1.25 | 2.01 | 0.25 |
| | PLACE2000216, highly | | | | | | | |
| | similar to SPECTRIN BETA | | | | | | | |
| | CHAIN, BRAIN | | | | | | | |
| 50 | hypothetical protein FLJ20037 | 142944 | R71124 | 3 | 4 | 2.10 | 1.74 | n/d |
| 51 | hypothetical protein FLJ20037 | 451087 | AA704517 | 3 | 4 | 2.36 | 1.73 | n/d |
| 52 | hypothetical protein | 417863 | W88781 | 5 | 6 | 2.18 | 1.85 | 0.95 |
| | DKFZp434P0116 | | | | | | | |
| 53 | KIAA0212 | 854874 | AA630346 | 7 | 8 | 1.77 | 1.84 | 0.84 |
| 54 | KIAA0914 | 283301 | N51424 | 9 | 10 | 1.93 | 1.40 | n/d |
| 55 | hypothetical protein FLJ20281 | 244686 | N54297 | 11 | 12 | 2.13 | 2.05 | n/d |
| 56 | KIAA0876 | 809806 | AA454753 | 13 | 14 | 2.80 | 1.82 | n/d |
| 57 | DKFZP586G1122 protein | 950778 | AA608636 | 17 | 18 | 1.76 | 1.99 | 0.44 |
| 58 | Putative zinc finger protein | 377452 | AA055692 | 19 | 20 | 2.16 | n/d | n/d |
| | LOC55818 | | | | | | | |
| 59 | hypothetical protein PRO0823 | 194965 | R88734 | 21 | 22 | 1.89 | 1.47 | 1.02 |
| 60 | hypothetical protein PRO0823 | 1486194 | AA936866 | 21 | 22 | 2.17 | 1.31 | 0.46 |
| 61 | cDNA DKFZp586H0324 clone | 130276 | R21170 | 61 | 62 | 2.07 | 2.47 | n/d |
| | DKFZp586H0324 | | | | | | | |
| 62 | Clone 23785 | 376476 | AA041362 | 63 | 64 | 2.50 | 2.29 | n/d |
| 63 | Clone 23785 | 261834 | H98855 | 63 | 64 | 2.14 | 1.97 | 0.49 |
| 64 | cDNA DKFZp586E1624 | 284497 | N52362 | 65 | 66 | 1.95 | 1.09 | n/d |
| 65 | ESTs (UniGene annotated) | 139558 | R62339 | 79 | 80 | 1.73 | 1.75 | 1.69 |
| 66 | ESTs (UniGene annotated) | 897446 | AA489477 | 81 | 82 | 1.74 | 1.83 | n/d |
| 67 | ESTs (UniGene annotated) | 126458 | R06601 | 83 | 84 | 2.53 | 1.92 | 2.07 |
| 68 | ESTs (UniGene annotated) | 122982 | R00332 | 85 | 86 | 2.43 | 3.96 | n/d |
| 69 | ESTs (UniGene annotated) | 811808 | AA463469 | 87 | 88 | 1.79 | 1.31 | 1.24 |
| 70 | ESTs (UniGene annotated) | 203544 | H56028 | 89 | 90 | 3.67 | 3.63 | n/d |
| 71 | ESTs (UniGene annotated) | 714437 | AA293300 | 91 | 92 | 2.46 | 1.85 | 3.47 |
| 72 | ESTs | 810448 | AA457116 | 67 | 68 | 4.87 | 2.97 | 1.22 |
| 73 | ESTs | 207275 | H59618 | 97 | 98 | 2.61 | 1.24 | 1.04 |
| 74 | ESTs | 785928 | AA449703 | 99 | 100 | 1.45 | 1.84 | 0.57 |
| 75 | ESTs | 827204 | AA521311 | 101 | 102 | 1.80 | 1.55 | 1.27 |
| 76 | ESTs | 343695 | W69170 | 103 | 104 | 1.78 | 1.48 | 1.69 |
| 77 | ESTs | 39145 | R51835 | 105 | 106 | 1.49 | 1.72 | 1.04 |
| 78 | ESTs | 220608 | H87770 | 107 | 108 | 1.47 | 2.09 | 1.09 |
| 79 | ESTs | 142087 | R69248 | 109 | 110 | 1.57 | 1.70 | n/d |
| 80 | ESTs | 82171 | T68844 | 111 | 112 | 2.44 | 2.16 | 1.19 |
| 81 | ESTs | 795325 | AA454177 | 113 | 114 | 1.75 | 1.28 | n/d |
| 82 | ESTs | 366966 | AA026562 | 115 | 116 | 1.27 | 1.70 | n/d |
| 83 | ESTs | 84419 | T73780 | 117 | 118 | 1.43 | 2.22 | 1.07 |
| 84 | ESTs | 742611 | AA401496 | 119 | 120 | 3.63 | 3.75 | n/d |
| 85 | ESTs | 277611 | N49384 | 119 | 120 | 4.52 | 2.87 | n/d |
| 86 | ESTs | 823688 | AA489636 | 121 | 122 | 2.11 | n/d | n/d |
| 87 | ESTs | 781311 | AA446361 | 123 | 124 | 1.66 | 2.43 | n/d |
| 88 | ESTs | 1555201 | AA931411 | 125 | 126 | 1.89 | n/d | n/d |
| 89 | ESTs | 131563 | R24223 | 127 | 128 | 2.26 | n/d | n/d |
| 90 | EST (singleton) | 786657 | AA451886 | 137 | 138 | 2.44 | 2.01 | n/d |
| 91 | ESTs (ex-UniGene) | 126393 | R06520 | 139 | 140 | 1.59 | 1.81 | 0.86 |
| 92 | ESTs (ex UniGene) | 74054 | T48278 | 141 | 142 | 1.92 | 1.05 | n/d |
| Legend | | | | | | | | |
| The last 3 columns show mRNA expression as a ratio between the conditions being compared. Of these three columns | | | | | | | | |
| # the first two show expression in hypoxia relative to normoxia, done separately in resting macrophages or activated | | | | | | | | |
| # macrophages. The final column shows expression in activated macrophages relative to resting macrophages (both in normoxia) | | | | | | | | |
| # as a ratio. n/d = not determined due to low signal intensities. IMAGE IDand accession descride the exact identity | | | | | | | | |
| # of the arrayed clones and do not describe full length cDNA sequence database entries. | | | | | | | | |

TABLE 8
Genes induced by hypoxia (greater response in resting cells)

| | | | | | | RATIO | | |
|---|---|---|---|---|---|---|---|---|
| | | | | SEQ ID | | Hypoxia/Normoxia | | Activated/Resting |
| Row | TITLE | IMAGE ID | accession | protein | nucl | (resting) | (activated) | (normoxia) |
| 1 | Metallothionein 1H | 214162 | H77766 | 239 | 240 | 6.26 | 2.01 | 17.58 |
| 2 | Metallothionein 1L | 297392 | N80129 | 241 | 242 | 18.55 | 2.21 | 7.57 |
| 3 | metallothionein 1L | 1899230 | AI289110 | 241 | 242 | 5.89 | 1.70 | 9.63 |
| 4 | Metallothionein-IG | 202535 | H53340 | 243 | 244 | 12.07 | 2.36 | 21.28 |
| 5 | Metallothionein 1E (functional) | 1472735 | AA872383 | 245 | 246 | 10.16 | 2.04 | 4.66 |
| 6 | RNAhelicase-related protein/ | 245990 | N55459 | 337 | 338 | 6.41 | 1.99 | 14.16 |
| | metallotheionein1F | | | | | | | |
| 7 | RNAhelicase-related protein/ | 78353 | T56281 | 337 | 338 | 5.19 | 1.54 | 12.00 |
| | metallotheionein1F | | | | | | | |
| 8 | Solute carrier family 2, member 3 | 753467 | AA406551 | 247 | 248 | 7.67 | 4.69 | 4.78 |
| 9 | Hexokinase 2 | 1637282 | AI005515 | 249 | 250 | 7.32 | 3.27 | 0.62 |
| 10 | DKFZp434E1723 clone | 1593887 | AA987423 | 69 | 70 | 2.04 | 1.34 | 1.49 |
| | DKFZp434E1723 | | | | | | | |
| 11 | CytochromeP450, subfamilyXXVI | 1761925 | AI222585 | 339 | 340 | 2.16 | 0.72 | 2.92 |
| | IB, polypept1 | | | | | | | |
| 12 | Interleukin 8 | 549933 | AA102526 | 251 | 252 | 5.65 | 0.86 | 382.80 |
| 13 | SHB adaptor protein | 768362 | AA495786 | 341 | 342 | 1.87 | 0.84 | 0.39 |
| 14 | ESTs | 130835 | R22252 | 129 | 130 | 1.97 | 0.93 | 0.83 |
| Legend to Table 8 | | | | | | | | |
| The last 3 columns show mRNA expression as a ratio between the conditions being compared. Of these three columns | | | | | | | | |
| # the first two show expression in hypoxia relative to normoxia, done separately in resting macrophages or activated | | | | | | | | |
| # macrophages. The final column shows expression in activated macrophages relative to resting macrophages (both in normoxia) | | | | | | | | |
| # as a ratio. n/d = not determined due to low signal intensities. IMAGE ID and accession describe the exact identity | | | | | | | | |
| # of the arrayed clones and do not describe full length cDNA sequence database entries. | | | | | | | | |

TABLE 9
Genes induced by hypoxia (greater response in activated cells)

| | | | | | RATIO | | |
|---|---|---|---|---|---|---|---|
| | IMAGE | | SEQ ID | | Hypoxia/Normoxia | | Activated/Resting |
| TITLE | ID | accession | protein | nucleotide | (resting) | (activated) | (normoxia) |
| Papillomavirus regulatory | 744983 | AA625924 | 343 | 344 | 3.36 | 8.10 | 0.22 |
| factor (PRF-1) | | | | | | | |
| CDNA FLJ11041 fis, clone | 140301 | R66924 | 71 | 72 | 1.46 | 3.19 | 2.50 |
| PLACE1004405 | | | | | | | |
| ESTs (ex-UniGene) | 139250 | R68736 | 143 | 144 | 1.01 | 2.18 | 1.68 |
| Legend | | | | | | | |
| The last 3 columns show mRNA expression as a ratio between the conditions being compared. Of these three columns | | | | | | | |
| # the first two show ecpression in hypoxia relative to normoxia, done separately in resting macrophages or activated | | | | | | | |
| # macrophages.The final column shows expression in activated macrophages relative to resting macrophages (both in normoxia) | | | | | | | |
| # as a ratio. n/d = not determined due to low signal intensities. IMAGE ID and accession describe the exact identity | | | | | | | |
| # of the arrayed clones and do not describe full length cDNA sequence database entries. | | | | | | | |

TABLE 10
Genes repressed by hypoxia (greater response in activated cells)

| | | | | | | RATIO | | |
|---|---|---|---|---|---|---|---|---|
| | | | | SEQ ID | | Hypoxia/Normoxia | | Activated/Resting |
| row | TITLE | IMAGE ID | accession | protein | nucl | (resting) | (activated) | (normoxia) |
| 1 | Maf-related leucine zipper | 77193 | T50121 | 457 | 458 | 1.18 | 0.48 | 2.39 |
| | homolog | | | | | | | |
| 2 | Alpha-2-macroglobulin | 44180 | H06516 | 405 | 406 | 1.11 | 0.54 | 1.98 |
| 3 | KIAA0014 | 725927 | AA292382 | 51 | 52 | 1.10 | 0.65 | 25.00 |
| 4 | ESTs | 178805 | H49601 | 203 | 204 | 1.04 | 0.49 | 1.62 |
| 5 | dynein, cytoplasmic, light | 811870 | AA454959 | 459 | 460 | 1.03 | 0.42 | 3.31 |
| | intermediate polypeptide 2 | | | | | | | |
| 6 | Heterochromatin-like protein 1 | 343490 | W69106 | 461 | 462 | 1.01 | 0.60 | 3.21 |
| 7 | Monocyte chemotactic protein 3 | 485989 | AA040170 | 463 | 464 | 0.89 | 0.52 | 59.62 |
| 8 | Fatty-acid-Coenzyme A ligase, | 82734 | T73556 | 465 | 466 | 0.88 | 0.52 | 6.85 |
| | long-chain 2 | | | | | | | |
| 9 | Fatty-acid-Coenzyme A ligase, | 2014138 | AI361530 | 465 | 466 | 0.72 | 0.46 | 3.97 |
| | long-chain 2 | | | | | | | |
| 10 | Programmed cell death 5/ | 502369 | AA156940 | 467 | 468 | 0.78 | 0.59 | 1.57 |
| | TFAR19 | | | | | | | |
| 11 | cDNA FLJ14028 fis, clone | 366156 | AA062814 | 145 | 146 | 0.75 | 0.55 | 1.74 |
| | HEMBA1003838 | | | | | | | |
| 12 | Small inducible cytokine A3 | 153355 | R47893 | 469 | 470 | 0.69 | 0.29 | 8.74 |
| 13 | Cytochrome c oxidase subunit | 42993 | R59927 | 471 | 472 | 0.72 | 0.54 | 1.98 |
| | VIc | | | | | | | |
| 14 | NASP histone-binding prot. | 845415 | AA644128 | 473 | 474 | 0.64 | 0.38 | 1.64 |
| 15 | hypothetical protein HSPC196 | 144902 | R78498 | 53 | 54 | 0.63 | 0.48 | 1.62 |
| 16 | Ecotropic viral integration site | 231675 | H93149 | 475 | 476 | 0.63 | 0.35 | 1.51 |
| | 2A | | | | | | | |
| 17 | Sjogren syndrome antigen B | 49970 | H29484 | 477 | 478 | 0.53 | 0.32 | 7.86 |
| 18 | Macrophage inflammatory | 205633 | H62985 | 407 | 408 | 0.52 | 0.28 | 12.73 |
| | protein 1b | | | | | | | |
| 19 | Monocyte chemotactic protein 1 | 768561 | AA425102 | 395 | 396 | 0.46 | 0.11 | 213.89 |
| 20 | Monocyte chemotactic protein 2 | 1911099 | AI268937 | 479 | 480 | undetectable | 0.26 | 423.31 |
| 21 | Endothelin 1 | 47359 | H11003 | 397 | 398 | undetectable | 0.56 | 14.29 |
| 22 | GRO2/macrophage | 153340 | R50407 | 481 | 482 | undetectable | 0.66 | 12.16 |
| | inflammatory protein 2a | | | | | | | |
| 23 | Small nuclear ribonucleoprotein | 47542 | H16454 | 483 | 484 | undetectable | 0.22 | 11.01 |
| | SM D 1 | | | | | | | |
| 24 | hypothetical protein FLJ11296 | 491460 | AA150443 | 55 | 56 | undetectable | 0.51 | 8.96 |
| 25 | GRO1/macrophage | 324437 | W46900 | 485 | 486 | undetectable | 0.48 | 15.29 |
| | inflammatory protein 2 | | | | | | | |
| | precursor | | | | | | | |
| 26 | GRO1/macrophage | 323238 | W42723 | 485 | 486 | undetectable | 0.40 | 7.92 |
| | inflammatory protein 2 | | | | | | | |
| | precursor | | | | | | | |
| 27 | Lymphocyte adhesion molecule | 149910 | H00756 | 487 | 488 | undetectable | 0.47 | 4.92 |
| | 1 | | | | | | | |
| 28 | Sex hormone-binding globulin | 82871 | T69346 | 409 | 410 | undetectable | 0.36 | 3.57 |
| 29 | ESTs | 898045 | AA598952 | 205 | 206 | undetectable | 0.53 | 2.37 |
| 30 | hypothetical protein bA395L14 | 842794 | AA486203 | 57 | 58 | undetectable | 0.45 | n/d |
| Legend to Table 10 | | | | | | | | |
| The last 3 columns show mRNA expression as a ratio between the conditions being compared. Of these three columns | | | | | | | | |
| # the first two show expression in hypoxia relative to normoxia, done separately in resting macrophages or activated | | | | | | | | |
| # macrophages. The final column shows expression in activated macrophages relative to resting macrophages (both in normoxia) | | | | | | | | |
| # as a ratio. n/d = not determined due to low signal intensities. IMAGE ID and accession descride the exact identity | | | | | | | | |
| # of the arrayed clones and do not describe full length cDNA sequence database entries. | | | | | | | | |

TABLE 11
Other genes repressed by hypoxia in macrophages

| | | | | | | RATIO | | |
|---|---|---|---|---|---|---|---|---|
| | | | | | | | | Activated/ |
| | | | | SEQ ID | | Hypoxia/Normoxia | | Resting |
| Row | TITLE | IMAGE ID | accession | PROTEIN | NUCL | (resting) | (activated) | (normoxia) |
| 1 | Annexin AI | 208718 | H63077 | 401 | 402 | 0.86 | 0.49 | 1.27 |
| 2 | ATP-binding cassette, sub-family E | 1593311 | AI002355 | 411 | 412 | 0.62 | 0.51 | 0.79 |
| | (OABP), 1 | | | | | | | |
| 3 | ESTs | 855583 | AA664228 | 165 | 166 | 0.61 | 0.52 | 0.73 |
| 4 | Chaperonin/Tcp zeta 1 | 45233 | H07880 | 413 | 414 | 0.59 | 0.55 | 1.27 |
| 5 | Chaperonin TCP1, subunit 6A zeta 1 | 45233 | H07880 | 413 | 414 | 0.72 | 0.52 | 1.00 |
| 6 | Colony stimulating factor 1 | 73527 | T55558 | 415 | 416 | 0.44 | 0.38 | 0.44 |
| | (macrophage) | | | | | | | |
| 7 | Colony stimulating factor 1 | 1475574 | AA878257 | 415 | 416 | 0.46 | 0.38 | 0.30 |
| | (macrophage) | | | | | | | |
| 8 | Dendritic cell protein (GA17) | 563634 | AA101348 | 417 | 418 | 0.59 | 0.53 | 0.97 |
| 9 | G protein-coupled receptor 44 | 810403 | AA464202 | 419 | 420 | 0.55 | 0.57 | 0.90 |
| 10 | Heat shock 70kD protein 4 | 856567 | AA633656 | 399 | 400 | 0.55 | n/d | n/d |
| 11 | Keratin 6A | 366481 | AA026418 | 421 | 422 | 0.53 | 1.21 | 0.25 |
| 12 | lymphocyte adaptor protein | 294196 | N71394 | 423 | 424 | 0.67 | 0.50 | 0.26 |
| 13 | Neuro-oncological ventral antigen 1 | 2015354 | AI362062 | 425 | 426 | 0.45 | 0.38 | 0.65 |
| 14 | N-SMase/FAN | 376644 | AA046107 | 427 | 428 | 0.59 | 0.86 | 0.18 |
| 15 | p67 myc protein | 812965 | AA464600 | 403 | 404 | 0.66 | 0.59 | 1.14 |
| 16 | Peptidylprolyl isomerase F | 774726 | AA442081 | 429 | 430 | 0.82 | 0.44 | n/d |
| | (cyclophilin F) | | | | | | | |
| 17 | PLECKSTRIN | 823779 | AA490267 | 431 | 432 | 0.76 | 0.52 | 1.30 |
| 18 | High affinity immunoglobulin | 199185 | R95749 | 433 | 434 | 0.65 | 0.59 | 1.22 |
| | epsilon receptor beta subunit | | | | | | | |
| 19 | High affinity immunoglobulin | 79576 | T62849 | 433 | 434 | 0.64 | 0.58 | 1.20 |
| | epsilon receptor beta subunit | | | | | | | |
| 20 | Ribosomal protein L44 | 884842 | AA669359 | 435 | 436 | 0.73 | 0.50 | 1.36 |
| 21 | Solute carrier family 6 No 1 | 177967 | H46254 | 437 | 438 | 0.62 | 0.54 | 1.38 |
| 22 | Synaptopodin | 178792 | H49443 | 439 | 440 | 0.54 | 0.56 | 1.24 |
| 23 | TERA protein | 1521977 | AA906997 | 441 | 442 | 0.44 | 0.34 | 0.46 |
| 24 | TGF beta-stimulated protein TSC-22 | 868630 | AA664389 | 443 | 444 | 0.56 | 0.72 | 0.83 |
| 25 | Tubulin, beta, 2 | 1492104 | AA888148 | 445 | 446 | 0.53 | 0.64 | 1.33 |
| 26 | Calgranulin A | 562729 | AA086471 | 447 | 448 | 0.57 | 0.65 | 16.86 |
| 27 | Replication factor C (145 KDa) | 214537 | H73714 | 449 | 450 | 0.66 | 0.50 | 2.80 |
| 28 | Signal recognition particle 19 kD | 754998 | AA411407 | 451 | 452 | 0.63 | 0.49 | 2.77 |
| | protein | | | | | | | |
| 29 | Nucleoside phosphorylase | 769890 | AA430382 | 393 | 394 | 0.50 | 0.79 | 2.75 |
| 30 | Transcription factor SUPT3H | 1606865 | AA996042 | 453 | 454 | 0.50 | 0.47 | 2.64 |
| 31 | Proteasome component C9 | 399536 | AA733040 | 455 | 456 | 0.64 | 0.58 | 2.09 |
| 32 | hypothetical nuclear factor SBBI22 | 868119 | AA634213 | 35 | 36 | 0.75 | 0.50 | 0.73 |
| 33 | DKFZP434I116 protein | 207379 | H58884 | 37 | 38 | 0.52 | 0.43 | 1.04 |
| 34 | hypothetical prot. FLJ10206 | 487921 | AA045286 | 39 | 40 | 0.61 | 1.00 | 0.24 |
| 35 | hypothetical protein FLJ10815 | 1506046 | AA905628 | 41 | 42 | 0.92 | 0.55 | 0.88 |
| 36 | hypothetical protein FLJ11100 | 811590 | AA454607 | 43 | 44 | 0.56 | 0.47 | 1.17 |
| 37 | hypothetical protein FLJ2064 | 868161 | AA633831 | 45 | 46 | 0.81 | 0.47 | 0.85 |
| 38 | hypothetical protein HSPC111 | 825695 | AA504814 | 47 | 48 | 0.43 | 0.44 | 1.05 |
| 39 | hypothetical protein LOC51251 | 379941 | AA778116 | 49 | 50 | 0.56 | 0.55 | 1.06 |
| 40 | hypothetical protein LOC51251 | 770997 | AA427715 | 49 | 50 | 0.56 | 0.74 | 1.47 |
| 41 | cDNA FLJ13016 fis, clone | 77483 | T58743 | 59 | 60 | 0.58 | 0.54 | 1.55 |
| | NT2RP3000624 | | | | | | | |
| 42 | DKFZp564D016 (clone | 824665 | AA482278 | 147 | 148 | 0.50 | n/d | n/d |
| | DKFZp564D016 | | | | | | | |
| 43 | cDNA FLJ11302 fis, clone | 108351 | T70612 | 149 | 150 | 0.75 | 0.49 | 1.36 |
| | PLACE1009971 | | | | | | | |
| 44 | NEDO FLJ10309 fis cl | 810026 | AA455267 | 151 | 152 | 0.57 | 1.20 | 0.28 |
| | NT2RM2000287 | | | | | | | |
| 45 | Sequence from clone RP11-394O2 | 122147 | T98503 | 153 | 154 | 0.51 | 0.99 | n/d |
| | on ch 20 | | | | | | | |
| 46 | ESTs (UniGene annotated) | 731255 | AA420992 | 155 | 156 | 0.67 | 0.53 | 0.86 |
| 47 | ESTs (UniGene annotated) | 434182 | AA693797 | 157 | 158 | 0.46 | 0.37 | 0.77 |
| 48 | ESTs (UniGene annotated) | 788415 | AA456437 | 159 | 160 | 0.50 | 0.52 | 0.89 |
| 49 | ESTs (UniGene annotated) | 49879 | H28725 | 159 | 160 | 0.35 | 0.38 | n/d |
| 50 | ESTs (UniGene annotated) | 770954 | AA429367 | 161 | 162 | 0.64 | 0.74 | 0.82 |
| 51 | ESTs (UniGene annotated) | 770935 | AA434382 | 163 | 164 | 0.55 | 0.98 | 0.51 |
| 52 | ESTs | 34626 | R44397 | 167 | 168 | 0.92 | 0.61 | 1.35 |
| 53 | ESTs | 1534589 | AA923509 | 169 | 170 | 0.70 | 0.48 | 0.72 |
| 54 | ESTs | 417223 | W87747 | 171 | 172 | 0.53 | 0.37 | 0.97 |
| 55 | ESTs | 854752 | AA630167 | 215 | 216 | 0.64 | 0.54 | 0.87 |
| 56 | ESTs | 1569263 | AA973568 | 173 | 174 | 0.52 | 0.45 | 0.62 |
| 57 | ESTs | 869440 | AA679939 | 213 | 214 | 0.60 | 0.53 | 0.78 |
| 58 | ESTs | 123065 | T98529 | 175 | 176 | 0.62 | 0.55 | 0.94 |
| 59 | ESTs | 364468 | AA022679 | 177 | 178 | 0.59 | 0.54 | 1.30 |
| 60 | ESTs | 50635 | H17921 | 179 | 180 | 0.45 | 0.43 | 0.94 |
| 61 | ESTs | 123858 | R00766 | 181 | 182 | 0.50 | 0.50 | 0.75 |
| 62 | ESTs | 415195 | W91958 | 183 | 184 | 0.54 | 0.59 | 1.28 |
| 63 | ESTs | 138865 | R63694 | 185 | 186 | 0.56 | 0.66 | 1.32 |
| 64 | ESTs | 773308 | AA425386 | 187 | 188 | 0.51 | 0.61 | 0.63 |
| 65 | ESTs | 1505857 | AA909912 | 189 | 190 | 0.46 | 0.78 | 0.83 |
| 66 | ESTs | 122728 | T99032 | 191 | 192 | 0.49 | 0.39 | 0.42 |
| 67 | ESTs | 202154 | H52503 | 193 | 194 | 0.35 | 0.42 | 0.35 |
| 68 | ESTs | 502634 | AA127017 | 195 | 196 | 0.52 | 0.88 | 0.54 |
| 69 | ESTs | 23005 | R38647 | 197 | 198 | 0.51 | n/d | n/d |
| 70 | ESTs | 22500 | T87233 | 199 | 200 | 0.55 | n/d | n/d |
| 71 | ESTs | 587398 | AA130351 | 201 | 202 | 0.52 | n/d | n/d |
| 72 | ESTs (ex-UniGene) | 1610469 | AA991868 | 207 | 208 | 0.54 | 0.53 | 0.41 |
| 73 | ESTs (ex-UniGene) | 81331 | T60111 | 209 | 210 | 0.92 | 0.46 | 0.24 |
| 74 | ESTs (ex-UniGene) | 1425266 | AA897090 | 211 | 212 | 0.55 | 0.75 | 0.19 |
| Legend | | | | | | | | |
| The last 3 columns show mRNA expression as a ratio between the conditions being compared. Of these three columns | | | | | | | | |
| # the first two show expression in hypoxia relative to normoxia, done separately in resting macrophages or activated | | | | | | | | |
| # macrophages. The final column shows expression in activated macrophages relative to resting macrophages (both in normoxia) | | | | | | | | |
| # as a ratio. n/d = not determined due to low signal intensities. IMAGE ID and accession descride the exact identity | | | | | | | | |
| # of the arrayed clones and do not describe full length cDNA sequence database entries. | | | | | | | | |

TABLE 12

| | | | #1 | #1 | | #2 | #2 | | #3 | #3 | | #4 | #4 | | #5 | | #6 |
|---|---|---|---|---|---|---|---|---|---|---|---|---|---|---|---|---|---|
| Seq | | #1 | HY | HY | #2 | HY | HY | #3 | HY | HY | #4 | HY | HY | #5 | HY | #6 | HY |
| ID | Clone | NO | 6 hr | 18 hr | NO | 6 hr | 18 hr | NO | 6 hr | 18 hr | NO | 6 hr | 18 hr | NO | 6 hr | NO | 6 hr |
| 2 | p1F12 | 1.26 | 0.83 | 1.12 | 2.59 | 2.41 | 2.84 | 1.36 | 1.42 | 1.75 | 0.41 | 0.35 | 0.41 | 0.70 | 1.59 | 0.48 | 0.46 |
| 4 | p1F2 | 1.39 | 2.37 | 1.74 | 1.18 | 1.78 | 1.62 | 0.74 | 0.67 | 0.50 | 0.80 | 1.12 | 1.24 | 1.30 | 1.98 | 0.94 | 1.38 |
| 6 | p1F10 | 1.41 | 1.07 | 0.83 | 1.57 | 1.71 | 1.71 | 1.60 | 1.92 | 1.67 | 0.79 | 0.66 | 0.56 | 0.69 | 1.48 | 1.17 | 0.94 |
| 8 | p1F19 | 0.54 | 0.53 | 0.78 | 0.99 | 1.04 | 1.25 | 1.18 | 1.23 | 1.22 | 0.44 | 0.54 | 0.72 | 0.37 | 0.29 | 0.94 | 0.67 |
| 10 | p1F8 | 0.63 | 1.34 | 1.17 | 0.87 | 1.04 | 1.04 | 0.53 | 1.07 | 1.09 | 0.50 | 1.20 | 1.96 | 0.32 | 0.99 | 0.74 | 1.21 |
| 12 | p1F5 | 1.41 | 1.04 | 0.98 | 1.12 | 1.63 | 1.35 | 2.09 | 2.40 | 1.91 | 0.78 | 0.65 | 0.88 | 0.81 | 1.67 | 0.83 | 0.75 |
| 14 | p1F18 | 1.34 | 0.69 | 1.02 | 1.43 | 1.65 | 1.90 | 1.98 | 1.62 | 2.17 | 0.74 | 0.55 | 0.72 | 0.40 | 0.57 | 0.74 | 0.70 |
| 16 | p1F7 | 0.69 | 0.35 | 0.75 | 1.83 | 1.35 | 1.67 | 1.99 | 5.27 | 2.95 | 1.05 | 0.91 | 0.71 | 0.47 | 0.47 | 0.05 | 0.08 |
| 18 | p1F21 | 0.68 | 0.58 | 0.50 | 0.85 | 0.85 | 0.74 | 0.80 | 0.66 | 0.52 | 0.61 | 0.66 | 0.63 | 0.27 | 0.59 | 2.16 | 5.39 |
| 20 | p1F9 | 0.36 | 0.47 | 0.86 | 0.95 | 2.47 | 0.09 | 3.60 | 2.70 | 3.01 | 0.43 | 0.93 | 1.02 | 0.23 | 0.54 | 0.29 | 0.62 |
| 22 | p1E13 | 1.76 | 1.30 | 0.95 | 1.04 | 1.78 | 1.01 | 0.59 | 0.66 | 0.55 | 0.89 | 0.65 | 0.72 | 1.04 | 1.50 | 3.73 | 3.36 |
| 24 | p1D1 | 0.88 | 1.98 | 5.49 | 1.47 | 2.03 | 3.59 | 1.32 | 2.69 | 2.05 | 0.63 | 2.45 | 5.51 | 0.94 | 1.79 | 0.10 | 0.33 |
| 24 | p1D2 | 1.03 | 0.99 | 3.89 | 1.76 | 3.50 | 4.79 | 0.73 | 1.87 | 1.78 | 1.07 | 2.22 | 4.16 | 0.68 | 1.01 | 0.13 | 0.23 |
| 26 | p1D4 | 0.41 | 1.88 | 1.95 | 0.83 | 1.80 | 2.19 | 1.57 | 1.80 | 1.54 | 0.42 | 1.42 | 1.50 | 0.49 | 2.36 | 0.31 | 0.39 |
| 28 | p1D9 | 1.16 | 0.13 | 0.93 | 1.52 | 0.80 | 0.85 | 4.04 | 4.86 | 2.60 | 1.23 | 1.45 | 1.28 | 0.65 | 1.14 | 1.37 | 0.94 |
| 30 | p1D12 | 5.56 | 4.42 | 3.24 | 0.62 | 3.70 | 1.69 | 0.54 | 0.77 | 0.78 | 1.09 | 1.13 | 0.96 | 1.36 | 1.03 | 3.70 | 4.01 |
| 32 | p1D15 | 0.81 | 0.54 | 1.48 | 1.10 | 1.39 | 1.05 | 0.74 | 0.86 | 0.69 | 0.77 | 0.90 | 0.81 | 0.38 | 0.73 | 0.54 | 0.65 |
| 34 | p1D16 | 0.44 | 1.99 | 0.84 | 1.11 | 1.29 | 1.22 | 2.07 | 2.43 | 1.61 | 0.42 | 0.78 | 1.32 | 0.61 | 1.18 | 0.66 | 0.98 |
| 36 | p1J13 | 1.15 | 0.62 | 0.54 | 1.24 | 0.91 | 0.84 | 1.94 | 1.73 | 1.66 | 1.08 | 0.75 | 0.50 | 0.66 | 0.69 | 0.51 | 0.39 |
| 38 | p1I22 | 0.72 | 0.65 | 1.20 | 1.97 | 1.77 | 1.49 | 1.57 | 1.60 | 1.75 | 0.95 | 0.90 | 0.86 | 0.95 | 1.59 | 1.10 | 0.74 |
| 40 | p1J6 | 0.91 | 0.96 | 1.04 | 1.08 | 0.99 | 0.96 | 0.97 | 1.21 | 0.87 | 0.99 | 1.04 | 1.07 | 1.18 | 1.60 | 1.10 | 1.29 |
| 42 | p1I5 | 3.02 | 1.24 | 2.56 | 1.56 | 3.17 | 1.47 | 1.46 | 1.76 | 1.21 | 1.16 | 1.32 | 0.93 | 0.54 | 0.88 | 0.73 | 1.07 |
| 44 | p1I13 | 8.65 | 6.65 | 3.11 | 0.71 | 3.20 | 0.89 | 0.96 | 1.11 | 0.84 | 1.92 | 1.66 | 1.08 | 1.25 | 0.71 | 2.09 | 2.43 |
| 46 | p1I17 | 0.88 | 0.55 | 0.63 | 2.03 | 1.13 | 1.38 | 1.93 | 4.01 | 2.52 | 1.16 | 0.92 | 0.88 | 0.23 | 0.32 | 0.67 | 0.51 |
| 48 | p1I15 | 2.06 | 1.31 | 0.49 | 1.19 | 0.53 | 0.43 | 1.88 | 1.66 | 0.97 | 1.47 | 0.74 | 0.91 | 1.24 | 1.86 | 0.61 | 0.34 |
| 50 | p1I7 | 0.79 | 0.56 | 0.64 | 1.20 | 0.69 | 0.80 | 2.13 | 3.28 | 1.74 | 0.91 | 0.96 | 0.99 | 0.47 | 0.65 | 0.57 | 0.34 |
| 54 | p1I4 | 1.26 | 0.51 | 0.35 | 1.21 | 0.95 | 0.47 | 1.04 | 1.45 | 0.93 | 0.90 | 0.43 | 0.29 | 0.73 | 0.82 | 1.40 | 1.18 |
| 56 | p1I8 | 4.53 | 3.14 | 2.83 | 1.51 | 2.71 | 1.50 | 0.89 | 1.04 | 0.76 | 1.24 | 1.09 | 1.00 | 0.65 | 1.04 | 0.93 | 1.26 |
| 58 | p1I16 | 0.70 | 0.51 | 0.41 | 2.31 | 1.46 | 1.60 | 3.37 | 2.91 | 2.73 | 0.51 | 0.51 | 0.43 | 0.76 | 0.64 | 1.03 | 0.82 |
| 60 | p1I11 | 0.63 | 0.62 | 0.34 | 1.64 | 1.36 | 1.19 | 2.75 | 1.62 | 1.38 | 0.63 | 0.37 | 0.40 | 0.59 | 0.84 | 0.56 | 0.51 |
| 62 | p1E8 | 0.67 | 0.47 | 0.71 | 1.41 | 1.14 | 1.22 | 1.59 | 3.29 | 1.99 | 1.08 | 1.07 | 0.95 | 0.25 | 0.40 | 0.52 | 0.48 |
| 64 | p1E18 | 1.26 | 1.07 | 0.72 | 1.45 | 2.07 | 1.10 | 0.77 | 0.93 | 0.73 | 0.93 | 0.71 | 0.80 | 0.57 | 1.23 | 6.36 | 5.38 |
| 66 | p1E16 | 0.84 | 0.56 | 1.06 | 0.96 | 0.95 | 0.88 | 1.90 | 6.76 | 5.00 | 2.43 | 2.67 | 2.25 | 0.32 | 0.49 | 0.62 | 0.81 |
| 68 | p1D5 | 1.71 | 1.21 | 1.40 | 1.58 | 1.81 | 1.66 | 2.13 | 1.93 | 2.05 | 0.84 | 0.79 | 0.81 | 0.78 | 0.90 | 0.75 | 0.80 |
| 68 | p1D6 | 0.24 | 1.42 | 2.51 | 0.28 | 0.61 | 0.93 | 1.77 | 1.57 | 1.85 | 0.25 | 1.35 | 2.82 | 0.34 | 0.52 | 0.61 | 1.49 |
| 70 | p1E12 | 0.54 | 0.41 | 0.62 | 0.98 | 1.29 | 0.67 | 0.98 | 1.47 | 1.13 | 0.83 | 0.84 | 0.87 | 1.11 | 1.17 | 0.59 | 1.01 |
| 72 | p1E10 | 1.09 | 0.63 | 1.03 | 4.31 | 3.17 | 5.08 | 2.27 | 4.17 | 3.76 | 1.03 | 1.01 | 1.06 | 0.75 | 0.74 | 0.74 | 0.79 |
| 74 | p1C21 | 3.92 | 2.75 | 2.31 | 0.93 | 2.33 | 0.92 | 1.81 | 2.27 | 1.45 | 0.91 | 0.96 | 0.95 | 0.41 | 0.33 | 2.10 | 2.68 |
| 76 | p1D10 | 0.45 | 0.88 | 1.49 | 0.60 | 1.80 | 1.63 | 1.34 | 2.61 | 2.82 | 0.34 | 1.48 | 1.83 | 0.65 | 1.21 | 0.36 | 0.49 |
| 78 | p1D13 | 2.93 | 1.83 | 2.94 | 0.80 | 3.69 | 1.10 | 1.51 | 3.28 | 3.02 | 0.89 | 1.59 | 1.30 | 0.67 | 0.64 | 0.50 | 0.75 |
| 80 | p1E9 | 1.54 | 0.89 | 1.48 | 1.18 | 1.80 | 1.53 | 0.68 | 0.82 | 0.60 | 0.60 | 0.64 | 0.73 | 0.92 | 1.62 | 4.20 | 3.97 |
| 82 | p1F1 | 0.58 | 0.33 | 0.52 | 1.42 | 1.19 | 1.14 | 1.64 | 1.70 | 1.49 | 0.95 | 0.63 | 0.54 | 0.37 | 0.50 | 0.62 | 0.61 |
| 84 | p1E7 | 1.20 | 1.29 | 1.63 | 0.12 | 0.18 | 0.22 | 1.40 | 1.03 | 0.71 | 2.36 | 2.17 | 2.85 | 4.89 | 6.75 | 0.08 | 1.07 |
| 86 | p1E6 | 1.36 | 1.09 | 1.01 | 0.79 | 0.92 | 1.04 | 0.79 | 1.35 | 1.04 | 1.32 | 1.87 | 1.63 | 3.25 | 5.97 | 0.34 | 0.74 |
| 88 | p2B1 | 1.51 | 0.97 | 0.80 | 2.39 | 2.31 | 2.25 | 1.06 | 1.22 | 1.18 | 1.10 | 0.77 | 0.51 | 0.72 | 1.19 | 0.60 | 0.50 |
| 90 | p1D14 | 0.67 | 0.82 | 1.64 | 0.60 | 3.42 | 1.80 | 1.78 | 5.24 | 3.57 | 1.07 | 2.33 | 2.11 | 0.68 | 0.67 | 0.29 | 0.74 |
| 92 | p1D17 | 6.32 | 5.30 | 3.77 | 0.46 | 3.08 | 1.47 | 0.41 | 0.60 | 0.43 | 1.05 | 0.92 | 0.91 | 1.17 | 1.13 | 3.73 | 3.80 |
| 92 | p1P14 | 0.43 | 0.66 | 0.74 | 1.22 | 1.60 | 1.14 | 1.00 | 1.53 | 1.32 | 0.49 | 0.73 | 1.15 | 0.43 | 0.69 | 0.21 | 0.45 |
| 94 | p1C24 | 0.92 | 0.88 | 0.53 | 1.76 | 1.65 | 1.37 | 1.08 | 1.09 | 0.89 | 1.23 | 1.07 | 0.85 | 0.68 | 1.28 | 0.60 | 0.77 |
| 96 | p1D3 | 0.62 | 1.56 | 1.13 | 1.83 | 0.85 | 0.73 | 3.09 | 4.01 | 3.43 | 0.29 | 0.52 | 0.71 | 0.15 | 0.28 | 5.72 | 5.67 |
| 98 | p1E14 | 1.09 | 1.92 | 0.89 | 1.93 | 1.46 | 0.96 | 1.23 | 1.58 | 0.96 | 0.88 | 0.71 | 1.26 | 0.87 | 1.68 | 0.67 | 0.89 |
| 100 | p1E20 | 0.63 | 0.61 | 1.12 | 1.47 | 1.34 | 0.99 | 1.45 | 1.94 | 1.49 | 0.68 | 0.88 | 0.98 | 0.19 | 0.34 | 0.36 | 0.43 |
| 102 | p2A24 | 1.21 | 0.65 | 0.67 | 2.42 | 2.21 | 2.48 | 2.35 | 3.05 | 2.59 | 1.10 | 0.76 | 0.68 | 0.68 | 0.91 | 0.28 | 0.22 |
| 104 | p1E17 | 0.94 | 0.56 | 0.99 | 0.95 | 1.55 | 0.96 | 1.93 | 2.41 | 2.37 | 0.89 | 0.88 | 0.80 | 0.53 | 0.73 | 0.84 | 1.15 |
| 106 | p1E19 | 0.79 | 0.65 | 0.62 | 2.64 | 2.37 | 2.99 | 2.10 | 1.61 | 1.77 | 0.61 | 0.40 | 0.64 | 0.42 | 0.89 | 0.68 | 0.76 |
| 108 | p1E15 | 2.82 | 1.46 | 2.04 | 1.42 | 2.65 | 1.00 | 0.92 | 1.10 | 0.78 | 1.83 | 2.00 | 1.73 | 0.82 | 0.72 | 1.21 | 1.35 |
| 110 | p1E11 | 1.36 | 1.12 | 0.87 | 2.09 | 2.86 | 3.03 | 0.74 | 0.91 | 0.88 | 0.89 | 0.59 | 0.79 | 0.77 | 2.09 | 2.76 | 2.97 |
| 112 | p1E23 | 1.19 | 0.65 | 1.55 | 1.24 | 2.11 | 1.42 | 0.75 | 1.15 | 1.02 | 0.86 | 1.09 | 1.09 | 0.35 | 0.40 | 0.31 | 0.36 |
| 114 | p1E21 | 0.82 | 0.52 | 0.85 | 2.08 | 2.28 | 2.04 | 2.14 | 2.04 | 2.27 | 0.74 | 0.49 | 0.79 | 0.51 | 1.04 | 0.48 | 0.67 |
| 116 | p1D23 | 0.95 | 0.47 | 0.68 | 1.21 | 1.62 | 1.30 | 0.99 | 1.17 | 1.10 | 1.39 | 1.20 | 1.05 | 0.35 | 0.49 | 0.28 | 0.27 |
| 118 | p1D24 | 0.98 | 0.71 | 1.39 | 1.55 | 1.98 | 2.16 | 1.18 | 1.47 | 1.01 | 0.74 | 0.49 | 0.67 | 1.53 | 1.94 | 0.90 | 0.98 |
| 120 | p1D22 | 0.70 | 0.89 | 1.98 | 0.89 | 2.66 | 2.13 | 0.71 | 1.40 | 1.45 | 0.72 | 1.61 | 2.10 | 0.26 | 0.65 | 0.22 | 0.96 |
| 122 | p1E2 | 3.39 | 1.91 | 1.92 | 1.56 | 1.78 | 1.65 | 1.11 | 1.09 | 1.03 | 3.44 | 3.32 | 1.96 | 0.68 | 0.78 | 0.76 | 0.73 |
| 124 | p1E1 | 2.91 | 1.31 | 2.14 | 3.07 | 6.21 | 2.25 | 1.08 | 2.11 | 1.28 | 1.02 | 1.01 | 1.21 | 0.56 | 0.80 | 0.64 | 1.16 |
| 126 | p1E4 | 0.65 | 0.51 | 0.79 | 1.06 | 1.24 | 1.18 | 1.59 | 1.57 | 1.70 | 0.80 | 0.76 | 0.91 | 0.34 | 0.39 | 0.97 | 1.13 |
| 128 | p1D18 | 8.78 | 5.01 | 4.93 | 1.12 | 5.44 | 2.18 | 0.58 | 0.80 | 0.50 | 1.25 | 1.25 | 0.99 | 1.15 | 1.18 | 1.34 | 1.93 |
| 130 | p1D21 | 1.56 | 0.98 | 0.96 | 1.18 | 2.30 | 1.47 | 0.48 | 0.78 | 0.60 | 0.67 | 0.60 | 0.59 | 0.40 | 0.63 | 0.91 | 1.44 |
| 132 | p1C22 | 3.70 | 2.08 | 2.77 | 0.94 | 2.43 | 1.34 | 0.92 | 1.26 | 0.74 | 1.85 | 1.25 | 1.49 | 0.91 | 0.77 | 2.36 | 2.86 |
| 134 | p1C23 | 0.91 | 0.93 | 0.88 | 1.84 | 1.17 | 1.36 | 3.92 | 3.52 | 2.77 | 1.02 | 1.07 | 1.63 | 0.60 | 0.73 | 0.67 | 0.54 |
| 136 | p1D11 | 0.54 | 1.09 | 1.35 | 0.73 | 1.57 | 1.74 | 2.78 | 4.49 | 3.68 | 0.60 | 1.22 | 1.63 | 0.80 | 1.83 | 0.22 | 0.74 |
| 138 | p1E3 | 2.55 | 0.54 | 0.61 | 6.62 | 1.75 | 1.81 | 0.34 | 0.15 | 0.09 | 1.15 | 0.42 | 0.25 | 0.28 | 0.41 | 2.49 | 2.94 |
| 140 | p1D20 | 0.75 | 0.93 | 1.16 | 1.35 | 1.72 | 1.18 | 1.63 | 2.62 | 2.78 | 0.57 | 0.54 | 0.75 | 0.43 | 0.69 | 0.66 | 0.77 |
| 142 | p1E5 | 0.86 | 0.56 | 0.60 | 1.59 | 1.42 | 1.41 | 0.94 | 1.32 | 0.94 | 0.41 | 0.82 | 0.56 | 6.88 | 11.0 | 2.01 | 5.20 |
| 144 | p1D19 | 3.52 | 1.95 | 2.39 | 1.10 | 3.29 | 1.54 | 0.96 | 1.23 | 0.82 | 1.87 | 1.69 | 1.80 | 0.59 | 0.66 | 1.50 | 2.04 |
| 146 | p2A15 | 0.89 | 0.64 | 0.65 | 2.04 | 1.91 | 1.67 | 2.70 | 3.56 | 3.33 | 1.25 | 1.20 | 1.00 | 0.74 | 0.69 | 0.44 | 0.50 |
| 148 | p1I14 | 0.96 | 0.54 | 0.67 | 1.30 | 0.68 | 1.11 | 2.36 | 2.45 | 1.69 | 1.10 | 0.90 | 0.47 | 0.20 | 0.24 | 0.38 | 0.26 |
| 150 | p1I2 | 1.89 | 1.15 | 1.70 | 2.50 | 3.12 | 2.26 | 1.57 | 1.92 | 1.47 | 0.73 | 0.60 | 0.85 | 0.46 | 0.66 | 0.95 | 0.63 |
| 152 | p1I12 | 2.00 | 1.52 | 1.68 | 0.91 | 1.02 | 1.40 | 0.66 | 0.88 | 0.62 | 1.81 | 1.36 | 1.48 | 1.96 | 2.49 | 2.13 | 2.37 |
| 154 | p1I3 | 1.26 | 0.94 | 0.55 | 1.53 | 1.29 | 1.14 | 1.13 | 1.40 | 1.20 | 0.85 | 0.50 | 0.42 | 0.70 | 0.89 | 1.04 | 0.70 |
| 156 | p1I10 | 1.82 | 1.30 | 0.97 | 1.15 | 1.65 | 1.18 | 0.59 | 0.72 | 0.53 | 1.89 | 1.64 | 1.05 | 0.75 | 1.07 | 0.76 | 0.71 |
| 158 | p1H18 | 0.52 | 0.65 | 1.16 | 0.83 | 0.67 | 0.23 | 1.49 | 1.33 | 1.35 | 1.34 | 1.21 | 0.97 | 1.51 | 2.07 | 0.70 | 0.67 |
| 160 | p1H24 | 7.02 | 5.55 | 3.73 | 0.83 | 3.66 | 1.22 | 1.04 | 1.32 | 0.77 | 1.27 | 1.71 | 1.53 | 0.89 | 0.92 | 3.02 | 4.58 |
| 162 | p1E22 | 0.48 | 0.30 | 0.53 | 1.93 | 2.28 | 2.13 | 1.68 | 1.65 | 2.38 | 0.41 | 0.35 | 0.49 | 0.35 | 0.46 | 1.26 | 1.00 |
| 164 | p1H21 | 0.82 | 0.56 | 0.66 | 3.63 | 2.47 | 3.49 | 0.83 | 0.96 | 0.82 | 0.64 | 0.67 | 0.76 | 5.64 | 10.2 | 6.34 | 16.3 |
| 166 | p1I1 | 1.64 | 0.90 | 1.61 | 1.02 | 1.71 | 0.85 | 1.13 | 1.33 | 0.97 | 0.88 | 0.85 | 0.77 | 0.78 | 0.84 | 1.43 | 2.04 |
| 168 | p1H14 | 0.86 | 0.45 | 0.71 | 1.62 | 1.10 | 0.78 | 1.65 | 1.91 | 1.77 | 0.87 | 0.87 | 1.00 | 0.32 | 0.46 | 0.39 | 0.37 |
| 170 | p1H11 | 0.96 | 0.63 | 0.72 | 2.19 | 2.53 | 2.46 | 2.53 | 3.11 | 3.04 | 0.74 | 0.67 | 0.79 | 0.46 | 0.87 | 1.11 | 0.78 |
| 172 | p1H17 | 0.58 | 0.69 | 1.17 | 0.65 | 0.64 | 0.17 | 1.67 | 1.53 | 1.54 | 1.36 | 1.39 | 1.11 | 1.30 | 1.73 | 0.63 | 0.71 |
| 174 | p1H12 | 2.06 | 1.50 | 1.29 | 1.79 | 2.33 | 2.36 | 0.84 | 1.09 | 0.87 | 0.80 | 0.63 | 0.69 | 0.82 | 2.34 | 0.96 | 0.78 |
| 176 | p1H7 | 0.70 | 0.64 | 1.12 | 1.21 | 1.38 | 0.75 | 1.66 | 1.47 | 1.38 | 1.10 | 1.04 | 0.79 | 1.27 | 1.86 | 0.52 | 0.55 |
| 178 | p1H15 | 2.34 | 1.79 | 1.89 | 0.91 | 1.77 | 0.73 | 0.92 | 0.85 | 0.57 | 1.17 | 0.80 | 0.76 | 1.10 | 1.14 | 1.43 | 2.36 |
| 180 | p1H20 | 0.42 | 0.53 | 0.98 | 0.91 | 0.81 | 0.24 | 1.47 | 1.48 | 1.33 | 1.26 | 1.07 | 0.88 | 1.04 | 1.83 | 0.73 | 0.76 |
| 182 | p1H8 | 0.86 | 0.87 | 1.87 | 1.02 | 1.13 | 0.22 | 2.17 | 2.37 | 1.65 | 2.56 | 2.34 | 1.63 | 1.93 | 2.30 | 1.00 | 0.97 |
| 184 | p1H16 | 0.68 | 0.42 | 0.54 | 1.59 | 1.56 | 1.68 | 1.28 | 1.01 | 0.97 | 0.25 | 0.14 | 0.29 | 1.47 | 2.48 | 0.62 | 0.81 |
| 186 | p1H9 | 1.31 | 0.64 | 0.55 | 0.94 | 0.74 | 0.46 | 1.88 | 2.01 | 1.52 | 1.05 | 0.97 | 1.02 | 0.25 | 0.40 | 0.45 | 0.35 |
| 188 | p1H23 | 1.10 | 0.60 | 0.60 | 1.28 | 0.97 | 0.67 | 1.96 | 2.81 | 2.33 | 1.20 | 0.93 | 0.86 | 0.40 | 0.52 | 0.39 | 0.34 |
| 190 | p1H10 | 7.36 | 4.40 | 4.02 | 1.57 | 7.80 | 2.18 | 0.85 | 1.11 | 0.73 | 0.97 | 0.82 | 0.98 | 1.07 | 1.42 | 2.62 | 3.20 |
| 192 | p1H6 | 8.14 | 6.12 | 3.46 | 0.48 | 4.02 | 1.26 | 0.41 | 0.47 | 0.29 | 1.51 | 1.62 | 1.06 | 1.84 | 1.16 | 4.77 | 5.40 |
| 194 | p1H13 | 0.59 | 2.10 | 1.03 | 0.83 | 0.58 | 0.21 | 2.87 | 1.67 | 1.71 | 0.49 | 0.64 | 1.22 | 3.85 | 5.85 | 1.99 | 1.55 |
| 196 | p1H19 | 0.47 | 0.46 | 0.54 | 1.12 | 0.54 | 0.16 | 2.12 | 2.58 | 1.50 | 1.53 | 1.40 | 1.04 | 1.65 | 1.82 | 1.03 | 0.80 |
| 198 | p1G22 | 1.14 | 0.78 | 0.72 | 2.75 | 2.67 | 1.41 | 8.35 | 12.7 | 8.79 | 1.35 | 1.21 | 1.01 | 0.59 | 0.70 | 0.77 | 0.63 |
| 200 | p1G21 | 0.99 | 1.01 | 0.55 | 2.02 | 1.48 | 1.10 | 1.54 | 1.13 | 0.80 | 0.78 | 0.65 | 0.60 | 0.72 | 1.35 | 1.28 | 0.69 |
| 202 | p1H1 | 1.33 | 0.82 | 0.82 | 2.50 | 2.70 | 1.59 | 0.94 | 1.26 | 1.95 | 1.22 | 1.06 | 0.98 | 0.81 | 0.87 | 0.65 | 0.82 |
| 204 | p1G20 | 0.74 | 0.48 | 0.50 | 1.54 | 0.66 | 0.36 | 1.64 | 1.26 | 1.03 | 0.99 | 0.84 | 0.84 | 1.34 | 1.19 | 1.29 | 0.98 |
| 206 | p1H5 | 2.46 | 1.48 | 1.52 | 1.40 | 3.58 | 1.14 | 5.93 | 16.5 | 11.3 | 0.97 | 0.81 | 0.87 | 0.80 | 1.19 | 0.92 | 1.18 |
| 208 | p1G19 | 0.44 | 0.52 | 0.79 | 0.88 | 0.81 | 0.30 | 1.75 | 1.77 | 1.18 | 1.41 | 1.37 | 1.06 | 1.76 | 2.43 | 0.75 | 0.64 |
| 210 | p1H2 | 1.13 | 0.55 | 0.49 | 0.84 | 0.83 | 0.39 | 2.34 | 4.04 | 4.03 | 0.68 | 0.56 | 0.47 | 0.12 | 0.17 | 2.43 | 2.35 |
| 212 | p1G18 | 0.41 | 0.82 | 0.75 | 0.95 | 0.63 | 0.22 | 2.50 | 2.28 | 1.45 | 0.59 | 1.02 | 1.02 | 1.67 | 2.11 | 1.29 | 0.91 |
| 214 | p1H4 | 1.61 | 1.00 | 1.44 | 0.92 | 1.89 | 0.63 | 1.15 | 1.46 | 1.00 | 1.26 | 1.15 | 1.02 | 0.90 | 0.66 | 1.11 | 1.21 |
| 216 | p1H3 | 1.28 | 0.68 | 1.11 | 1.13 | 1.55 | 0.48 | 0.87 | 0.90 | 0.56 | 1.01 | 0.77 | 0.66 | 1.05 | 0.88 | 2.01 | 3.04 |
| 222 | p1P3 | 1.32 | 0.79 | 1.45 | 1.39 | 2.10 | 1.47 | 17.1 | 10.7 | 10.8 | 0.82 | 1.02 | 1.15 | 0.53 | 0.83 | 0.48 | 1.08 |
| 224 | p1A9 | 1.09 | 2.80 | 2.72 | 1.01 | 1.15 | 2.43 | 3.64 | 5.80 | 3.79 | 0.32 | 0.88 | 1.62 | 0.59 | 1.33 | 0.24 | 0.44 |
| 224 | p1A8 | 0.52 | 11.0 | 1.43 | 0.60 | 0.57 | 0.83 | 4.46 | 4.51 | 2.81 | 0.17 | 0.42 | 1.28 | 1.75 | 4.84 | 0.99 | 2.32 |
| 226 | p1B17 | 1.04 | 24.7 | 2.25 | 1.16 | 1.22 | 1.25 | 1.08 | 0.99 | 0.81 | 0.69 | 0.81 | 0.79 | 1.50 | 2.35 | 2.53 | 2.56 |
| 228 | p1O20 | 0.58 | 1.42 | 5.10 | 0.73 | 4.10 | 7.70 | 0.63 | 0.84 | 1.02 | 0.74 | 2.32 | 2.75 | 0.35 | 0.96 | 0.06 | 0.10 |
| 230 | p1B2 | 0.66 | 1.96 | 5.58 | 0.58 | 1.02 | 1.50 | 1.53 | 0.95 | 0.87 | 0.57 | 2.55 | 4.40 | 0.56 | 1.20 | 0.25 | 1.03 |
| 232 | p1B3 | 1.24 | 1.35 | 3.82 | 1.78 | 2.17 | 4.64 | 3.30 | 3.42 | 2.96 | 0.79 | 1.01 | 1.32 | 0.10 | 0.26 | 0.15 | 0.40 |
| 236 | p1B19 | 1.34 | 1.54 | 6.26 | 1.28 | 0.89 | 2.48 | 9.63 | 9.26 | 6.08 | 0.66 | 2.09 | 5.36 | 0.72 | 1.85 | 0.01 | 0.03 |
| 236 | p1B18 | 0.65 | 18.3 | 2.73 | 1.68 | 1.06 | 2.54 | 26.2 | 10.0 | 4.27 | 0.19 | 0.57 | 5.12 | 2.30 | 16.1 | 0.16 | 0.34 |
| 238 | p1N17 | 2.81 | 1.63 | 2.01 | 1.24 | 3.19 | 1.83 | 4.80 | 0.87 | 0.57 | 1.48 | 1.29 | 1.16 | 0.47 | 0.59 | 1.06 | 1.27 |
| 240 | p1A24 | 4.49 | 3.48 | 2.62 | 0.34 | 1.91 | 1.06 | 0.94 | 0.99 | 0.59 | 1.36 | 1.63 | 1.94 | 6.97 | 14.2 | 1.24 | 2.41 |
| 244 | p1B1 | 1.45 | 1.74 | 2.07 | 0.14 | 0.16 | 0.32 | 1.08 | 1.32 | 0.86 | 1.03 | 1.09 | 1.69 | 5.95 | 24.7 | 0.04 | 1.06 |
| 248 | p1A4 | 0.63 | 0.55 | 0.78 | 2.47 | 3.35 | 4.34 | 2.21 | 1.60 | 3.18 | 0.92 | 0.59 | 1.17 | 0.30 | 0.55 | 0.35 | 0.33 |
| 248 | p1A2 | 0.57 | 0.81 | 1.07 | 1.36 | 1.62 | 1.87 | 1.48 | 1.73 | 1.65 | 0.44 | 0.48 | 1.02 | 0.33 | 0.65 | 0.51 | 1.49 |
| 248 | p1A3 | 0.55 | 1.18 | 0.80 | 0.56 | 1.04 | 1.29 | 1.42 | 1.47 | 1.06 | 0.56 | 0.92 | 1.40 | 0.20 | 0.36 | 0.37 | 1.88 |
| 248 | p1A1 | 0.58 | 1.50 | 0.66 | 1.06 | 1.04 | 1.19 | 1.35 | 1.20 | 1.07 | 0.77 | 1.65 | 0.82 | 0.59 | 1.31 | 0.53 | 1.76 |
| 250 | p1A16 | 0.75 | 1.16 | 0.95 | 1.36 | 1.29 | 1.26 | 1.22 | 1.65 | 1.37 | 0.66 | 0.89 | 0.74 | 0.63 | 1.20 | 0.44 | 0.92 |
| 250 | p1A15 | 0.49 | 1.03 | 1.93 | 0.27 | 1.07 | 1.07 | 0.67 | 1.54 | 1.03 | 0.39 | 1.66 | 1.22 | 0.04 | 0.10 | 0.79 | 3.65 |
| 250 | p1A18 | 0.60 | 0.86 | 1.64 | 0.28 | 1.02 | 0.96 | 0.70 | 1.44 | 1.22 | 0.46 | 1.52 | 1.35 | 0.23 | 0.26 | 0.96 | 4.48 |
| 250 | p1A17 | 0.78 | 2.06 | 0.51 | 1.32 | 1.13 | 0.93 | 0.79 | 1.03 | 1.54 | 0.64 | 1.17 | 0.74 | 0.47 | 0.67 | 1.01 | 6.98 |
| 252 | p1B14 | 0.78 | 1.20 | 5.92 | 6.75 | 3.77 | 5.37 | 1.13 | 5.81 | 6.57 | 0.17 | 0.33 | 0.66 | 2.16 | 4.85 | 0.10 | 0.12 |
| 252 | p1B15 | 1.22 | 1.15 | 7.37 | 14.4 | 11.6 | 11.0 | 0.92 | 6.90 | 6.45 | 0.40 | 0.61 | 0.94 | 1.89 | 3.76 | 0.08 | 0.10 |
| 252 | p1B16 | 1.14 | 0.97 | 7.33 | 13.6 | 10.7 | 9.97 | 1.04 | 7.34 | 7.64 | 0.45 | 0.65 | 1.03 | 1.99 | 3.48 | 0.09 | 0.10 |
| 254 | p1A12 | 0.44 | 0.91 | 1.17 | 1.07 | 0.92 | 1.33 | 4.01 | 3.77 | 3.56 | 0.77 | 1.59 | 2.04 | 0.37 | 0.55 | 0.44 | 0.70 |
| 254 | p1A11 | 0.16 | 3.67 | 0.42 | 0.58 | 0.42 | 0.57 | 2.87 | 1.45 | 0.95 | 0.20 | 0.43 | 2.01 | 1.85 | 5.50 | 1.17 | 5.82 |
| 256 | p1A13 | 0.34 | 2.14 | 1.10 | 0.42 | 0.36 | 0.65 | 3.57 | 3.81 | 3.15 | 0.19 | 0.79 | 1.98 | 0.34 | 0.60 | 1.24 | 1.86 |
| 258 | p1A14 | 0.27 | 5.92 | 0.30 | 0.40 | 0.45 | 0.71 | 7.23 | 2.92 | 2.30 | 0.08 | 0.18 | 1.06 | 2.39 | 6.30 | 4.94 | 7.07 |
| 260 | p1A19 | 0.56 | 2.88 | 0.45 | 0.99 | 0.90 | 1.33 | 2.11 | 1.14 | 0.73 | 0.45 | 0.53 | 0.82 | 1.46 | 4.56 | 4.90 | 1.37 |
| 262 | p1A20 | 0.20 | 8.18 | 0.34 | 0.36 | 0.40 | 0.68 | 14.1 | 8.24 | 6.34 | 0.06 | 0.27 | 1.66 | 1.38 | 3.74 | 2.34 | 3.16 |
| 264 | p1A22 | 0.78 | 1.44 | 2.34 | 0.52 | 1.40 | 1.52 | 2.59 | 0.81 | 2.38 | 0.46 | 1.47 | 1.92 | 0.77 | 0.98 | 0.19 | 0.56 |
| 266 | p1A23 | 0.71 | 2.10 | 1.41 | 0.07 | 0.06 | 0.13 | 3.12 | 2.80 | 1.63 | 1.06 | 1.94 | 2.95 | 7.39 | 18.1 | 0.03 | 0.95 |
| 268 | p1B21 | 3.08 | 1.82 | 2.69 | 0.59 | 3.22 | 0.71 | 0.56 | 0.66 | 0.41 | 1.48 | 1.45 | 0.75 | 0.77 | 0.99 | 96.6 | 117 |
| 268 | p1B20 | 0.72 | 0.85 | 0.77 | 1.17 | 1.27 | 1.05 | 0.74 | 1.03 | 0.57 | 0.80 | 0.77 | 0.64 | 0.61 | 1.25 | 1505 | 1742 |
| 270 | p1C17 | 0.38 | 0.91 | 0.47 | 1.39 | 0.63 | 1.21 | 3.27 | 2.63 | 2.74 | 0.37 | 0.28 | 0.59 | 0.21 | 0.18 | 3.94 | 4.47 |
| 270 | p1C18 | 0.42 | 0.76 | 0.51 | 1.51 | 0.99 | 1.69 | 2.88 | 2.45 | 2.64 | 0.32 | 0.31 | 0.54 | 0.16 | 0.27 | 4.76 | 5.23 |
| 272 | p1D8 | 2.74 | 2.48 | 2.75 | 0.99 | 2.72 | 1.61 | 0.74 | 0.78 | 0.74 | 1.31 | 1.08 | 1.01 | 0.84 | 0.91 | 2.08 | 2.03 |
| 274 | p1A10 | 0.13 | 1.74 | 1.78 | 0.33 | 2.02 | 1.93 | 3.00 | 3.12 | 2.80 | 0.20 | 1.69 | 2.86 | 0.06 | 0.27 | 0.30 | 1.81 |
| 276 | p1G24 | 1.41 | 1.31 | 1.91 | 3.09 | 4.24 | 2.63 | 0.92 | 1.60 | 0.93 | 1.14 | 1.39 | 1.75 | 0.14 | 0.30 | 0.40 | 0.70 |
| 278 | p1G23 | 2.81 | 1.41 | 1.97 | 1.06 | 2.80 | 1.23 | 0.90 | 1.14 | 0.62 | 0.94 | 0.85 | 0.80 | 0.37 | 0.51 | 2.52 | 1.37 |
| 280 | p1G5 | 0.34 | 0.86 | 1.46 | 0.56 | 1.70 | 1.61 | 0.63 | 1.16 | 1.35 | 0.43 | 1.23 | 1.77 | 0.21 | 0.53 | 0.21 | 0.97 |
| 282 | p1G7 | 0.74 | 0.61 | 0.80 | 1.25 | 1.26 | 1.03 | 1.13 | 1.77 | 1.39 | 0.87 | 1.28 | 1.54 | 0.37 | 0.56 | 0.36 | 0.61 |
| 284 | p2A23 | 1.79 | 1.24 | 1.15 | 1.81 | 2.26 | 1.79 | 1.11 | 1.28 | 0.95 | 1.26 | 0.87 | 0.77 | 0.77 | 1.43 | 0.76 | 0.91 |
| 286 | p1G1 | 0.54 | 0.39 | 0.81 | 1.68 | 2.29 | 0.15 | 1.05 | 1.41 | 1.33 | 1.85 | 2.65 | 1.71 | 0.35 | 0.58 | 0.21 | 0.33 |
| 288 | p1G15 | 7.04 | 5.26 | 3.09 | 0.70 | 3.29 | 2.10 | 0.57 | 0.77 | 0.55 | 2.10 | 1.82 | 1.03 | 1.54 | 1.44 | 3.27 | 3.54 |
| 290 | p1F23 | 0.90 | 0.42 | 1.09 | 1.89 | 1.13 | 1.09 | 1.22 | 2.57 | 1.79 | 1.10 | 0.88 | 0.70 | 0.42 | 0.55 | 0.33 | 0.40 |
| 292 | p1G8 | 1.02 | 0.70 | 1.33 | 0.86 | 3.03 | 1.31 | 1.15 | 3.36 | 2.20 | 1.04 | 1.93 | 1.56 | 0.33 | 0.71 | 0.23 | 0.35 |
| 294 | p1G13 | 8.69 | 6.51 | 4.23 | 1.06 | 4.37 | 2.12 | 0.37 | 0.47 | 0.36 | 1.39 | 1.41 | 0.82 | 1.03 | 1.17 | 5.78 | 7.17 |
| 296 | p1G10 | 1.01 | 0.61 | 1.52 | 1.69 | 1.76 | 1.65 | 2.01 | 3.58 | 2.49 | 1.32 | 1.13 | 0.99 | 0.44 | 0.65 | 0.44 | 0.62 |
| 298 | p1F24 | 1.17 | 0.70 | 1.01 | 9.63 | 5.52 | 8.15 | 0.56 | 0.87 | 0.56 | 12.8 | 11.5 | 14.2 | 0.12 | 0.15 | 0.11 | 0.14 |
| 300 | p1G2 | 1.69 | 1.00 | 1.36 | 5.01 | 4.65 | 3.35 | 1.04 | 1.47 | 0.83 | 1.28 | 1.39 | 1.24 | 0.36 | 1.37 | 0.42 | 0.48 |
| 302 | p1G11 | 0.53 | 0.42 | 0.42 | 0.55 | 0.86 | 0.65 | 1.43 | 3.83 | 3.56 | 0.45 | 0.36 | 0.43 | 1.21 | 1.55 | 0.29 | 0.53 |
| 304 | p1G16 | 0.53 | 0.29 | 0.55 | 1.58 | 1.64 | 1.65 | 1.10 | 2.51 | 1.64 | 0.87 | 0.64 | 0.71 | 0.41 | 0.74 | 0.23 | 0.34 |
| 306 | p1G9 | 1.32 | 0.61 | 0.77 | 1.07 | 1.78 | 0.79 | 0.74 | 1.22 | 0.96 | 0.95 | 0.85 | 0.66 | 0.79 | 1.09 | 0.94 | 1.06 |
| 308 | p1G4 | 0.83 | 0.42 | 0.92 | 1.67 | 3.65 | 9.50 | 0.69 | 0.62 | 0.54 | 1.48 | 0.87 | 1.17 | 0.49 | 0.74 | 0.71 | 1.39 |
| 310 | p1G14 | 8.40 | 5.55 | 4.36 | 0.70 | 4.34 | 1.75 | 0.47 | 0.56 | 0.36 | 1.14 | 0.98 | 0.90 | 1.30 | 1.11 | 3.49 | 4.03 |
| 312 | p1A6 | 0.30 | 0.59 | 2.57 | 0.31 | 1.10 | 1.88 | 1.02 | 1.31 | 1.44 | 0.56 | 1.50 | 2.81 | 0.16 | 0.24 | 0.23 | 0.80 |
| 312 | p1A5 | 5.61 | 2.87 | 4.93 | 1.15 | 4.00 | 2.95 | 0.60 | 0.87 | 0.47 | 1.77 | 1.64 | 1.79 | 0.56 | 0.55 | 1.70 | 3.28 |
| 314 | p1B9 | 1.14 | 19.9 | 3.20 | 0.86 | 2.05 | 1.01 | 1.01 | 1.35 | 1.08 | 0.63 | 3.53 | 6.52 | 0.75 | 2.19 | 1.66 | 2.87 |
| 314 | p1B6 | 0.69 | 18.7 | 1.14 | 0.59 | 0.72 | 0.92 | 0.70 | 0.54 | 0.61 | 0.59 | 0.89 | 1.01 | 1.54 | 4.45 | 2.13 | 3.09 |
| 314 | p1B8 | 1.11 | 35.1 | 0.81 | 0.74 | 1.23 | 1.02 | 1.15 | 1.35 | 0.91 | 0.57 | 1.27 | 3.41 | 0.85 | 3.57 | 2.53 | 3.71 |
| 314 | p1B7 | 0.60 | 34.9 | 1.17 | 0.59 | 0.74 | 0.80 | 0.90 | 0.80 | 0.72 | 0.48 | 0.84 | 1.76 | 1.94 | 4.62 | 4.60 | 7.31 |
| 316 | p1G17 | 0.85 | 1.53 | 0.92 | 1.10 | 0.83 | 1.05 | 2.04 | 1.86 | 1.78 | 1.40 | 2.19 | 2.22 | 0.81 | 0.87 | 0.46 | 0.74 |
| 318 | p1G3 | 0.38 | 0.39 | 1.02 | 0.76 | 1.16 | 0.87 | 1.38 | 1.89 | 1.71 | 0.81 | 0.73 | 1.00 | 0.81 | 2.03 | 0.24 | 0.48 |
| 320 | p1F22 | 0.94 | 0.56 | 0.75 | 1.15 | 1.24 | 1.60 | 2.56 | 3.11 | 2.46 | 1.10 | 1.70 | 1.67 | 0.45 | 0.47 | 0.29 | 0.31 |
| 322 | p1G12 | 0.29 | 0.41 | 0.96 | 0.99 | 1.95 | 1.56 | 0.80 | 1.75 | 1.22 | 0.54 | 0.87 | 1.24 | 0.12 | 0.31 | 0.16 | 0.30 |
| 324 | p1F11 | 1.49 | 0.94 | 1.01 | 1.16 | 1.64 | 1.56 | 0.64 | 0.91 | 0.71 | 0.84 | 0.69 | 1.00 | 0.77 | 1.16 | 0.53 | 1.01 |
| 326 | p1F16 | 2.63 | 0.62 | 0.65 | 6.26 | 1.77 | 1.43 | 0.39 | 0.16 | 0.13 | 1.07 | 0.39 | 0.30 | 0.29 | 0.30 | 2.73 | 3.22 |
| 328 | p1F14 | 0.75 | 0.39 | 0.89 | 0.97 | 0.92 | 0.84 | 1.29 | 1.35 | 1.36 | 0.93 | 0.90 | 1.03 | 0.54 | 0.98 | 0.16 | 0.22 |
| 330 | p1F17 | 1.52 | 0.95 | 1.82 | 5.53 | 3.68 | 5.49 | 0.24 | 0.32 | 0.33 | 2.14 | 2.44 | 2.18 | 0.86 | 1.66 | 0.22 | 0.92 |
| 332 | p1C2 | 0.26 | 0.31 | 0.31 | 1.00 | 1.28 | 1.53 | 5.09 | 13.6 | 16.1 | 0.17 | 0.21 | 0.31 | 0.19 | 0.37 | 0.67 | 1.53 |
| 334 | p1F3 | 1.64 | 1.06 | 1.20 | 1.52 | 1.52 | 1.41 | 1.05 | 1.55 | 1.28 | 1.26 | 1.02 | 0.99 | 0.71 | 1.05 | 0.46 | 0.50 |
| 336 | p1F20 | 0.69 | 1.35 | 1.32 | 1.10 | 1.16 | 1.03 | 1.73 | 2.66 | 2.42 | 0.34 | 0.64 | 0.76 | 0.86 | 1.82 | 0.53 | 0.87 |
| 338 | p1F6 | 1.92 | 1.67 | 2.74 | 0.11 | 0.17 | 0.28 | 1.73 | 1.30 | 1.02 | 1.80 | 2.33 | 2.74 | 5.33 | 7.33 | 0.04 | 0.73 |
| 340 | p1F4 | 1.50 | 1.06 | 0.87 | 1.14 | 1.59 | 1.15 | 0.68 | 1.05 | 0.83 | 0.92 | 0.72 | 0.66 | 0.94 | 1.85 | 2.78 | 6.20 |
| 342 | p1F15 | 1.29 | 0.82 | 1.10 | 1.97 | 1.87 | 1.33 | 1.71 | 1.75 | 1.30 | 0.62 | 0.78 | 0.74 | 0.34 | 0.81 | 0.63 | 0.83 |
| 344 | p1F13 | 0.32 | 1.18 | 2.65 | 0.54 | 3.15 | 2.25 | 0.74 | 1.19 | 1.19 | 0.80 | 3.41 | 4.43 | 0.21 | 0.49 | 0.17 | 0.52 |
| 346 | p1A7 | 0.74 | 0.60 | 0.50 | 0.87 | 0.63 | 0.80 | 0.85 | 0.95 | 0.69 | 0.71 | 0.57 | 0.76 | 0.73 | 1.22 | 1.59 | 1.11 |
| 348 | p1A21 | 1.35 | 0.89 | 1.29 | 1.27 | 1.17 | 0.98 | 0.85 | 1.49 | 1.32 | 1.07 | 1.31 | 1.17 | 0.52 | 0.49 | 0.18 | 0.22 |
| 350 | p1B5 | 1.08 | 0.94 | 3.22 | 1.73 | 1.39 | 3.45 | 2.08 | 2.24 | 1.78 | 0.34 | 0.55 | 1.57 | 0.10 | 0.17 | 0.07 | 0.13 |
| 350 | p1B4 | 0.71 | 7.10 | 2.98 | 2.43 | 1.47 | 3.12 | 10.3 | 7.97 | 5.70 | 0.48 | 0.93 | 2.72 | 0.65 | 1.12 | 0.44 | 0.75 |
| 352 | p1B12 | 1.74 | 1.15 | 1.23 | 0.70 | 1.53 | 0.93 | 0.60 | 0.57 | 0.30 | 0.94 | 0.88 | 0.89 | 0.34 | 0.37 | 1.15 | 1.50 |
| 352 | p1B11 | 0.21 | 0.17 | 0.40 | 1.25 | 1.64 | 1.18 | 1.14 | 0.66 | 0.45 | 0.94 | 1.02 | 1.31 | 0.28 | 0.30 | 0.27 | 0.64 |
| 352 | p1B10 | 0.46 | 0.97 | 0.71 | 1.31 | 0.94 | 1.06 | 1.92 | 2.27 | 1.82 | 0.51 | 0.43 | 0.63 | 0.86 | 2.71 | 0.44 | 0.99 |
| 354 | p1B13 | 0.93 | 0.99 | 1.06 | 0.85 | 1.01 | 0.91 | 0.77 | 0.97 | 0.68 | 1.00 | 1.03 | 1.02 | 1.22 | 1.61 | 1.20 | 1.42 |
| 356 | p1B22 | 3.21 | 1.60 | 3.89 | 1.04 | 0.81 | 0.97 | 3.14 | 4.98 | 4.06 | 1.67 | 1.55 | 1.15 | 0.20 | 0.21 | 0.30 | 0.34 |
| 358 | p1B23 | 0.36 | 0.52 | 1.08 | 0.80 | 1.09 | 1.17 | 0.93 | 0.66 | 0.85 | 0.27 | 0.52 | 0.82 | 1.05 | 1.83 | 17.2 | 12.5 |
| 360 | p1B24 | 0.54 | 0.24 | 0.51 | 0.67 | 0.52 | 1.09 | 1.88 | 2.32 | 1.98 | 0.65 | 0.54 | 0.70 | 0.34 | 0.34 | 0.86 | 1.59 |
| 362 | p1C3 | 0.71 | 0.68 | 0.77 | 2.10 | 0.92 | 1.22 | 4.25 | 2.15 | 1.97 | 1.07 | 1.18 | 1.09 | 0.46 | 0.54 | 0.48 | 0.57 |
| 364 | p1C4 | 0.80 | 0.81 | 0.75 | 1.82 | 1.94 | 1.74 | 1.18 | 1.08 | 1.02 | 0.88 | 0.77 | 0.78 | 0.73 | 1.04 | 0.79 | 1.02 |
| 366 | p1C5 | 0.52 | 0.67 | 1.21 | 0.93 | 0.89 | 1.07 | 1.80 | 2.92 | 2.03 | 0.70 | 1.42 | 1.68 | 0.58 | 1.01 | 0.61 | 0.85 |
| 368 | p1C6 | 0.41 | 3.01 | 0.46 | 0.86 | 0.85 | 0.76 | 1.67 | 1.69 | 1.30 | 0.51 | 0.72 | 0.81 | 0.97 | 2.40 | 1.15 | 4.75 |
| 370 | p1C7 | 0.93 | 2.60 | 0.63 | 0.74 | 0.89 | 0.75 | 3.35 | 1.93 | 1.49 | 0.61 | 0.93 | 0.83 | 1.81 | 2.86 | 1.92 | 1.25 |
| 372 | p1C8 | 0.11 | 0.51 | 0.83 | 1.17 | 1.79 | 2.54 | 1.93 | 2.11 | 1.46 | 0.07 | 0.82 | 0.84 | 0.61 | 1.54 | 0.89 | 0.77 |
| 374 | p1C9 | 1.04 | 1.00 | 1.67 | 1.31 | 1.62 | 1.61 | 1.18 | 2.32 | 1.60 | 0.75 | 0.86 | 0.89 | 0.42 | 0.57 | 0.57 | 0.60 |
| 376 | p1C10 | 0.65 | 0.58 | 0.43 | 1.29 | 1.10 | 1.42 | 0.93 | 1.48 | 1.17 | 1.07 | 0.93 | 0.84 | 0.68 | 0.84 | 14.6 | 15.2 |
| 378 | p1C11 | 0.69 | 1.30 | 0.86 | 1.59 | 0.56 | 0.90 | 2.64 | 2.63 | 2.42 | 0.45 | 0.52 | 1.32 | 0.79 | 1.37 | 0.90 | 0.59 |
| 380 | p1C12 | 1.00 | 1.39 | 0.78 | 1.31 | 1.07 | 1.31 | 2.18 | 1.86 | 1.29 | 0.75 | 0.91 | 1.05 | 0.97 | 2.57 | 0.55 | 0.88 |
| 382 | p1C13 | 0.61 | 5.60 | 1.61 | 0.78 | 0.80 | 0.90 | 3.98 | 3.29 | 2.33 | 0.73 | 2.76 | 6.00 | 0.54 | 1.10 | 1.24 | 1.66 |
| 384 | p1C14 | 0.44 | 3.47 | 0.52 | 0.64 | 0.96 | 0.89 | 4.73 | 3.06 | 1.92 | 0.59 | 0.69 | 1.91 | 1.10 | 2.27 | 1.64 | 2.85 |
| 386 | p1C15 | 1.40 | 0.82 | 0.84 | 0.50 | 0.65 | 0.50 | 2.02 | 6.84 | 2.20 | 2.00 | 1.39 | 1.64 | 0.37 | 0.42 | 0.46 | 0.58 |
| 388 | p1C16 | 2.84 | 1.76 | 2.30 | 0.95 | 1.80 | 1.11 | 0.95 | 5.76 | 6.09 | 1.57 | 1.27 | 0.98 | 0.66 | 0.68 | 1.25 | 1.79 |
| 390 | p1C19 | 2.33 | 1.63 | 1.78 | 0.58 | 0.90 | 0.67 | 0.73 | 1.35 | 1.04 | 0.98 | 0.97 | 0.98 | 0.33 | 0.33 | 13.7 | 17.4 |
| 392 | p1C20 | 0.68 | 1.08 | 1.96 | 1.56 | 1.12 | 1.04 | 2.89 | 4.45 | 4.18 | 0.71 | 1.25 | 1.76 | 0.31 | 0.47 | 0.27 | 0.48 |
| 396 | p1P5 | 3.26 | 1.18 | 1.71 | 70.5 | 33.8 | 21.0 | 1.93 | 6.73 | 7.47 | 1.84 | 2.20 | 1.55 | 0.75 | 0.67 | 1.12 | 0.57 |
| 398 | p2L23 | 0.99 | 0.66 | 0.57 | 63.1 | 35.3 | 56.2 | 245 | 289 | 339 | 0.47 | 0.50 | 0.42 | 0.47 | 1.04 | 0.30 | 0.32 |
| 402 | p1K9 | 1.09 | 0.61 | 1.08 | 1.23 | 0.55 | 1.15 | 1.42 | 2.46 | 2.34 | 2.23 | 1.48 | 1.83 | 0.01 | 0.01 | 0.18 | 0.24 |
| 404 | p1K23 | 1.91 | 1.08 | 1.43 | 0.91 | 0.60 | 0.75 | 1.67 | 1.98 | 1.51 | 2.01 | 1.52 | 1.89 | 0.72 | 0.72 | 0.18 | 0.18 |
| 406 | p1K15 | 2.31 | 1.24 | 1.63 | 1.23 | 2.38 | 1.19 | 0.89 | 1.09 | 0.82 | 1.11 | 0.96 | 0.84 | 2.57 | 2.98 | 13.2 | 14.6 |
| 408 | p1K8 | 1.32 | 0.86 | 0.76 | 0.97 | 1.35 | 1.29 | 1.08 | 1.98 | 1.56 | 0.98 | 0.71 | 0.72 | 0.94 | 1.46 | 3.51 | 1.11 |
| 410 | p1M24 | 1.71 | 0.94 | 1.37 | 1.68 | 2.31 | 1.49 | 1.24 | 1.49 | 1.29 | 0.81 | 0.74 | 1.10 | 0.42 | 0.75 | 1.22 | 0.69 |
| 412 | p1K7 | 2.24 | 0.79 | 0.56 | 1.84 | 0.69 | 0.58 | 2.40 | 4.45 | 2.78 | 2.16 | 1.57 | 1.05 | 0.62 | 0.51 | 0.46 | 0.21 |
| 414 | p1K16 | 1.23 | 0.58 | 0.37 | 1.14 | 0.64 | 0.49 | 2.19 | 2.31 | 1.76 | 1.05 | 0.69 | 0.64 | 0.48 | 0.50 | 0.41 | 0.19 |
| 416 | p1K18 | 3.59 | 1.63 | 1.92 | 1.02 | 2.11 | 1.23 | 1.07 | 1.33 | 0.92 | 2.28 | 1.71 | 1.51 | 0.89 | 0.76 | 1.94 | 1.43 |
| 418 | p1N1 | 0.83 | 0.42 | 0.54 | 1.45 | 0.67 | 0.85 | 1.38 | 1.52 | 1.32 | 1.28 | 0.96 | 1.03 | 0.24 | 0.36 | 0.24 | 0.13 |
| 420 | p1K22 | 1.82 | 1.13 | 0.74 | 0.90 | 0.73 | 0.51 | 2.20 | 2.31 | 1.76 | 1.27 | 0.91 | 1.03 | 0.55 | 0.61 | 0.37 | 0.26 |
| 422 | p1K14 | 0.87 | 0.94 | 1.02 | 1.06 | 0.96 | 0.95 | 1.39 | 1.74 | 1.21 | 0.96 | 1.02 | 1.08 | 1.14 | 1.56 | 1.10 | 1.30 |
| 424 | p1K13 | 1.70 | 0.82 | 1.59 | 1.14 | 0.76 | 1.10 | 6.27 | 4.09 | 4.13 | 3.03 | 2.53 | 2.33 | 0.43 | 0.46 | 1.33 | 0.75 |
| 426 | p1J20 | 1.07 | 0.84 | 1.33 | 0.87 | 1.05 | 0.29 | 1.50 | 1.73 | 1.35 | 1.30 | 1.44 | 0.95 | 1.27 | 1.84 | 0.87 | 0.58 |
| 428 | p1J22 | 2.11 | 2.14 | 1.90 | 1.00 | 1.69 | 1.41 | 0.74 | 0.86 | 0.59 | 1.95 | 1.68 | 1.34 | 2.55 | 2.75 | 2.32 | 2.40 |
| 430 | p1K1 | 1.09 | 1.27 | 0.65 | 1.74 | 0.79 | 0.67 | 1.18 | 1.46 | 1.08 | 0.47 | 0.46 | 0.56 | 1.06 | 1.93 | 1.11 | 0.70 |
| 432 | p1K3 | 1.18 | 1.11 | 0.83 | 1.19 | 1.95 | 1.20 | 0.61 | 0.78 | 0.47 | 0.97 | 0.83 | 0.75 | 1.00 | 1.54 | 55.5 | 32.9 |
| 434 | p1J19 | 1.62 | 1.24 | 0.85 | 1.63 | 2.03 | 2.49 | 0.50 | 0.73 | 0.54 | 0.76 | 0.58 | 0.68 | 0.99 | 1.77 | 7.90 | 4.54 |
| 434 | p1K2 | 1.06 | 0.80 | 0.92 | 1.67 | 1.95 | 1.52 | 0.41 | 0.82 | 0.39 | 0.94 | 0.87 | 0.83 | 0.78 | 0.83 | 10.1 | 5.86 |
| 436 | p1K5 | 0.93 | 0.43 | 0.81 | 0.93 | 0.54 | 0.54 | 1.58 | 1.27 | 1.28 | 1.24 | 0.89 | 1.19 | 0.23 | 0.31 | 0.17 | 0.18 |
| 438 | p1J17 | 0.93 | 0.72 | 0.95 | 1.11 | 1.25 | 0.49 | 0.93 | 0.65 | 0.39 | 1.09 | 0.58 | 0.45 | 0.95 | 1.11 | 2.10 | 2.41 |
| 440 | p1J18 | 0.31 | 0.18 | 0.36 | 1.10 | 0.53 | 0.26 | 1.04 | 0.44 | 0.24 | 0.94 | 0.43 | 0.44 | 1.35 | 1.30 | 2.83 | 4.02 |
| 442 | p1J15 | 0.55 | 0.66 | 1.26 | 0.99 | 0.68 | 0.22 | 1.68 | 1.41 | 1.25 | 1.75 | 1.55 | 1.24 | 1.47 | 2.14 | 0.69 | 0.65 |
| 444 | p1K4 | 0.41 | 0.21 | 0.23 | 1.61 | 1.07 | 1.06 | 1.38 | 1.29 | 0.96 | 0.90 | 0.65 | 0.68 | 0.67 | 1.07 | 0.66 | 0.35 |
| 446 | p2A14 | 2.61 | 2.13 | 1.22 | 0.87 | 1.15 | 1.12 | 0.95 | 1.36 | 0.83 | 2.04 | 1.59 | 1.21 | 1.97 | 2.23 | 1.18 | 1.21 |
| 448 | p1J23 | 0.96 | 0.57 | 0.50 | 1.05 | 1.18 | 1.38 | 0.73 | 1.01 | 0.70 | 0.54 | 0.42 | 0.54 | 0.94 | 1.33 | 10.5 | 5.19 |
| 450 | p1J21 | 0.75 | 0.68 | 0.96 | 1.16 | 1.09 | 0.52 | 1.61 | 1.35 | 1.35 | 1.15 | 0.96 | 0.79 | 0.98 | 1.60 | 0.56 | 0.66 |
| 452 | p1J24 | 1.62 | 0.56 | 0.84 | 1.98 | 1.51 | 0.96 | 1.56 | 2.04 | 1.37 | 1.05 | 0.81 | 0.52 | 0.56 | 0.62 | 0.30 | 0.27 |
| 454 | p1J16 | 3.62 | 1.55 | 2.31 | 1.38 | 2.76 | 1.29 | 2.85 | 4.20 | 3.66 | 1.65 | 1.84 | 1.78 | 0.53 | 0.76 | 1.04 | 0.62 |
| 456 | p1J2 | 0.77 | 0.76 | 0.37 | 1.57 | 0.75 | 0.51 | 2.52 | 3.12 | 2.29 | 0.38 | 0.28 | 0.45 | 0.72 | 1.14 | 0.63 | 0.30 |
| 458 | p1J9 | 0.73 | 0.45 | 0.38 | 1.22 | 1.38 | 1.06 | 2.10 | 1.00 | 0.87 | 0.38 | 0.34 | 0.34 | 0.71 | 1.18 | 3.04 | 3.92 |
| 460 | p1J10 | 0.86 | 0.48 | 0.72 | 1.18 | 1.10 | 0.85 | 1.70 | 2.20 | 1.82 | 1.20 | 0.92 | 1.08 | 0.26 | 0.32 | 0.62 | 0.48 |
| 462 | p1J1 | 0.97 | 0.70 | 0.68 | 1.41 | 1.09 | 0.87 | 3.29 | 3.09 | 2.75 | 0.63 | 0.45 | 0.51 | 0.63 | 0.86 | 0.55 | 0.46 |
| 464 | p1J5 | 2.26 | 0.94 | 0.74 | 56.2 | 22.5 | 16.6 | 1.12 | 2.88 | 2.58 | 1.22 | 0.79 | 0.83 | 1.06 | 1.15 | 3.02 | 1.07 |
| 466 | p1J11 | 0.95 | 0.57 | 0.30 | 0.71 | 0.51 | 0.60 | 0.95 | 0.70 | 0.70 | 0.37 | 0.22 | 0.18 | 0.86 | 1.20 | 1.77 | 0.97 |
| 468 | p1J8 | 1.77 | 0.67 | 1.20 | 1.73 | 0.98 | 0.66 | 1.16 | 1.88 | 1.28 | 1.70 | 1.59 | 1.57 | 0.28 | 0.34 | 0.11 | 0.08 |
| 470 | p1I20 | 2.84 | 1.42 | 1.67 | 1.17 | 2.14 | 1.41 | 0.99 | 1.46 | 1.03 | 0.79 | 0.72 | 0.92 | 0.74 | 0.90 | 7.59 | 2.80 |
| 472 | p1J3 | 0.70 | 0.39 | 0.64 | 1.75 | 1.28 | 1.04 | 1.24 | 1.61 | 1.81 | 0.94 | 0.69 | 0.85 | 0.33 | 0.52 | 0.53 | 0.51 |
| 474 | p1J12 | 7.58 | 4.86 | 4.02 | 0.90 | 4.17 | 1.83 | 0.84 | 1.06 | 0.78 | 1.06 | 0.88 | 0.90 | 1.01 | 0.94 | 3.33 | 4.41 |
| 476 | p1I23 | 1.35 | 0.87 | 0.71 | 1.36 | 2.00 | 1.43 | 0.71 | 1.03 | 0.70 | 0.80 | 1.12 | 1.23 | 0.41 | 0.84 | 2.67 | 1.69 |
| 478 | p1J7 | 1.54 | 0.65 | 0.90 | 1.22 | 1.15 | 0.65 | 1.34 | 1.20 | 1.06 | 1.50 | 1.07 | 0.97 | 0.26 | 0.31 | 0.40 | 0.28 |
| 480 | p1I21 | 1.76 | 0.96 | 0.81 | 31.8 | 11.1 | 9.16 | 1.33 | 2.83 | 2.80 | 1.14 | 0.85 | 0.92 | 1.16 | 1.74 | 1.19 | 0.42 |
| 482 | p1I19 | 1.15 | 0.74 | 0.71 | 11.42 | 5.80 | 5.51 | 0.83 | 2.40 | 2.91 | 0.82 | 0.66 | 0.94 | 6.67 | 8.50 | 0.41 | 0.34 |
| 484 | p1J4 | 1.92 | 0.80 | 0.63 | 2.15 | 0.85 | 0.63 | 3.22 | 7.24 | 4.65 | 2.55 | 1.77 | 1.05 | 0.91 | 1.08 | 0.13 | 0.07 |
| 486 | p1I24 | 3.43 | 1.85 | 1.70 | 20.5 | 11.56 | 9.6 | 0.51 | 1.70 | 1.90 | 1.00 | 0.84 | 0.98 | 2.75 | 3.80 | 0.67 | 0.77 |
| 488 | p1I18 | 1.27 | 0.84 | 0.91 | 6.72 | 5.15 | 4.95 | 1.01 | 1.60 | 1.28 | 0.81 | 0.81 | 0.67 | 0.80 | 1.49 | 0.85 | 0.58 |

| | | #6 | | #7 | #7 | | #8 | #8 | | #9 | #9 | | #10 | #10 | | #11 | #11 |
|---|---|---|---|---|---|---|---|---|---|---|---|---|---|---|---|---|---|
| Seq | | HY | #7 | HY | HY | #8 | HY | HY | #9 | HY | HY | #10 | HY | HY | #11 | HY | HY |
| ID | Clone | 18 hr | NO | 6 hr | 18 hr | NO | 6 hr | 18 hr | NO | 6 hr | 18 hr | NO | 6 hr | 18 hr | NO | 6 hr | 18 hr |
| 2 | p1F12 | 0.71 | 0.41 | 0.45 | 0.26 | 0.42 | 0.70 | 0.65 | 1.55 | 1.34 | 1.68 | 0.80 | 1.06 | 0.95 | 1.21 | 1.68 | 1.38 |
| 4 | p1F2 | 1.29 | 0.83 | 1.55 | 0.94 | 1.42 | 1.92 | 4.03 | 0.73 | 0.57 | 0.87 | 0.31 | 0.33 | 0.42 | 0.69 | 1.03 | 0.79 |
| 6 | p1F10 | 1.50 | 0.98 | 2.39 | 0.54 | 0.59 | 0.77 | 0.87 | 1.01 | 0.88 | 0.72 | 0.77 | 0.91 | 0.76 | 1.85 | 1.90 | 1.62 |
| 8 | p1F19 | 0.71 | 2.69 | 4.29 | 1.99 | 0.57 | 0.87 | 1.12 | 1.21 | 1.79 | 1.07 | 1.16 | 1.57 | 1.56 | 1.06 | 0.98 | 0.93 |
| 10 | p1F8 | 1.69 | 0.60 | 2.33 | 0.81 | 1.22 | 2.39 | 3.16 | 0.91 | 0.91 | 1.82 | 0.33 | 1.03 | 1.98 | 1.33 | 2.23 | 3.62 |
| 12 | p1F5 | 1.46 | 0.57 | 1.93 | 0.41 | 0.96 | 1.83 | 2.23 | 0.85 | 0.86 | 0.75 | 0.63 | 0.81 | 0.81 | 2.06 | 2.83 | 1.93 |
| 14 | p1F18 | 0.90 | 0.31 | 0.40 | 0.27 | 0.78 | 1.21 | 1.00 | 1.33 | 1.41 | 1.00 | 1.16 | 1.67 | 1.54 | 1.04 | 1.01 | 1.04 |
| 16 | p1F7 | 0.05 | 0.81 | 0.86 | 0.52 | 0.01 | 0.03 | 0.02 | 1.87 | 1.16 | 1.17 | 3.87 | 5.09 | 6.00 | 1.13 | 1.59 | 1.52 |
| 18 | p1F21 | 6.25 | 3.00 | 5.81 | 5.25 | 2.78 | 6.02 | 8.14 | 1.90 | 2.11 | 0.96 | 0.87 | 1.43 | 1.29 | 0.92 | 1.34 | 1.01 |
| 20 | p1F9 | 0.81 | 0.66 | 2.81 | 2.93 | 0.30 | 4.24 | 1.98 | 1.26 | 3.69 | 6.69 | 0.93 | 2.81 | 4.90 | 0.76 | 1.30 | 1.41 |
| 22 | p1E13 | 3.44 | 0.77 | 0.91 | 0.52 | 4.94 | 5.66 | 7.44 | 0.80 | 0.76 | 0.83 | 0.48 | 0.48 | 0.53 | 1.25 | 1.37 | 1.45 |
| 24 | p1D1 | 0.79 | 0.84 | 1.45 | 1.75 | 0.06 | 0.08 | 0.15 | 0.18 | 0.21 | 2.61 | 0.41 | 1.22 | 2.15 | 0.52 | 1.53 | 2.90 |
| 24 | p1D2 | 0.36 | 0.45 | 0.69 | 1.35 | 0.14 | 0.14 | 0.20 | 0.91 | 1.82 | 6.00 | 0.71 | 2.33 | 2.12 | 1.15 | 2.37 | 4.15 |
| 26 | p1D4 | 0.96 | 4.30 | 30.5 | 28.2 | 0.07 | 0.88 | 1.59 | 0.17 | 0.22 | 2.68 | 0.96 | 4.76 | 10.5 | 0.28 | 1.01 | 0.69 |
| 28 | p1D9 | 0.93 | 2.44 | 6.15 | 2.16 | 0.70 | 0.60 | 0.74 | 0.54 | 0.39 | 0.50 | 0.91 | 1.08 | 1.35 | 0.74 | 0.78 | 0.77 |
| 30 | p1D12 | 5.05 | 0.20 | 0.50 | 0.28 | 1.23 | 1.20 | 1.70 | 0.25 | 0.35 | 0.36 | 0.27 | 0.76 | 1.01 | 0.94 | 1.09 | 1.10 |
| 32 | p1D15 | 1.10 | 1.18 | 1.98 | 1.14 | 0.33 | 0.44 | 0.53 | 1.73 | 1.72 | 1.69 | 2.13 | 4.23 | 8.70 | 1.24 | 1.31 | 1.89 |
| 34 | p1D16 | 1.42 | 1.67 | 4.88 | 1.93 | 0.70 | 0.86 | 1.12 | 0.33 | 0.24 | 2.10 | 0.73 | 1.20 | 1.81 | 0.48 | 0.88 | 1.28 |
| 36 | p1J13 | 0.46 | 1.61 | 2.09 | 0.97 | 0.58 | 0.55 | 0.60 | 2.18 | 1.53 | 0.88 | 1.41 | 1.42 | 1.10 | 1.49 | 1.21 | 0.96 |
| 38 | p1I22 | 0.82 | 0.58 | 0.59 | 0.16 | 0.48 | 1.16 | 0.82 | 1.45 | 0.97 | 0.85 | 0.87 | 0.96 | 1.00 | 2.15 | 1.26 | 1.26 |
| 40 | p1J6 | 1.48 | 0.60 | 0.33 | 0.39 | 1.79 | 1.70 | 1.50 | 0.88 | 1.23 | 0.75 | 0.63 | 0.48 | 0.75 | 0.98 | 0.93 | 0.99 |
| 42 | p1I5 | 0.86 | 0.52 | 1.00 | 0.31 | 0.35 | 0.56 | 0.55 | 0.90 | 1.09 | 0.56 | 0.81 | 0.65 | 0.38 | 1.71 | 2.18 | 1.36 |
| 44 | p1I13 | 3.69 | 0.66 | 0.22 | 0.09 | 0.80 | 0.86 | 0.71 | 1.71 | 0.85 | 0.82 | 1.23 | 0.92 | 0.65 | 0.77 | 0.81 | 0.53 |
| 46 | p1I17 | 0.64 | 0.74 | 0.71 | 0.42 | 0.54 | 0.75 | 0.73 | 1.67 | 1.42 | 2.09 | 1.44 | 1.48 | 1.22 | 2.14 | 2.56 | 1.78 |
| 48 | p1I15 | 0.31 | 2.06 | 1.98 | 0.44 | 0.29 | 0.24 | 0.22 | 2.30 | 1.31 | 0.96 | 1.57 | 0.94 | 0.41 | 0.94 | 0.80 | 0.45 |
| 50 | p1I7 | 0.37 | 1.86 | 1.54 | 1.42 | 0.28 | 0.36 | 0.49 | 3.38 | 2.22 | 2.73 | 2.24 | 2.29 | 2.35 | 1.19 | 0.94 | 0.91 |
| 54 | p1I4 | 1.05 | 1.83 | 1.14 | 0.40 | 0.74 | 0.78 | 0.93 | 2.06 | 1.20 | 0.52 | 1.57 | 0.90 | 0.39 | 1.25 | 1.21 | 1.06 |
| 56 | p1I8 | 1.50 | 0.31 | 0.27 | 0.27 | 0.46 | 0.50 | 0.59 | 0.90 | 1.14 | 0.84 | 0.36 | 0.50 | 0.56 | 1.24 | 1.76 | 1.35 |
| 58 | p1I16 | 0.99 | 10.6 | 17.9 | 4.96 | 0.51 | 0.73 | 0.42 | 0.83 | 0.79 | 0.42 | 2.34 | 3.52 | 2.38 | 1.54 | 1.61 | 1.06 |
| 60 | p1I11 | 0.57 | 1.13 | 1.47 | 0.49 | 0.64 | 0.61 | 0.55 | 3.68 | 2.80 | 2.80 | 1.54 | 1.51 | 1.07 | 1.89 | 1.81 | 1.62 |
| 62 | p1E8 | 0.66 | 1.23 | 1.31 | 0.97 | 0.41 | 0.63 | 0.57 | 1.93 | 1.25 | 1.44 | 1.50 | 2.35 | 2.34 | 0.98 | 1.19 | 0.98 |
| 64 | p1E18 | 6.48 | 0.46 | 0.48 | 0.34 | 2.58 | 2.30 | 3.06 | 1.14 | 0.78 | 0.72 | 0.51 | 0.62 | 0.68 | 1.56 | 1.91 | 1.78 |
| 66 | p1E16 | 1.01 | 3.17 | 3.38 | 1.75 | 0.16 | 0.53 | 0.90 | 0.18 | 0.22 | 0.17 | 0.75 | 1.05 | 1.15 | 1.25 | 1.08 | 1.31 |
| 68 | p1D5 | 0.98 | 0.47 | 0.82 | 0.51 | 0.52 | 0.74 | 0.71 | 1.13 | 0.91 | 1.23 | 1.18 | 1.58 | 1.21 | 1.05 | 1.24 | 1.13 |
| 68 | p1D6 | 2.56 | 1.76 | 11.9 | 9.56 | 0.51 | 1.35 | 2.15 | 0.10 | 0.13 | 1.08 | 0.39 | 1.28 | 3.59 | 0.26 | 0.39 | 1.00 |
| 70 | p1E12 | 1.03 | 0.67 | 1.66 | 0.89 | 0.53 | 0.88 | 1.07 | 1.27 | 1.41 | 1.18 | 1.21 | 1.59 | 1.86 | 1.11 | 1.20 | 1.06 |
| 72 | p1E10 | 0.91 | 1.62 | 1.31 | 0.79 | 0.60 | 0.78 | 0.79 | 0.74 | 0.70 | 0.48 | 2.12 | 0.88 | 1.20 | 1.37 | 1.53 | 1.27 |
| 74 | p1C21 | 2.86 | 0.60 | 0.66 | 0.49 | 0.60 | 0.65 | 0.85 | 0.41 | 0.49 | 0.15 | 1.16 | 1.65 | 0.93 | 1.09 | 1.12 | 1.00 |
| 76 | p1D10 | 0.89 | 1.45 | 2.68 | 2.94 | 0.35 | 0.35 | 0.53 | 0.75 | 1.64 | 3.48 | 0.80 | 4.59 | 6.54 | 0.50 | 1.17 | 1.22 |
| 78 | p1D13 | 0.82 | 0.50 | 0.76 | 0.45 | 0.15 | 0.27 | 0.42 | 0.52 | 2.31 | 2.24 | 1.08 | 3.73 | 2.25 | 0.92 | 2.02 | 2.80 |
| 80 | p1E9 | 6.19 | 0.25 | 0.42 | 0.18 | 2.33 | 3.63 | 5.01 | 0.93 | 0.80 | 0.85 | 0.57 | 0.61 | 0.61 | 1.58 | 1.37 | 1.17 |
| 82 | p1F1 | 0.51 | 0.91 | 0.90 | 0.30 | 0.34 | 0.53 | 0.66 | 1.66 | 1.23 | 1.06 | 1.46 | 1.58 | 1.03 | 1.25 | 1.24 | 1.06 |
| 84 | p1E7 | 0.86 | 0.87 | 2.06 | 1.32 | 0.03 | 0.75 | 1.20 | 0.04 | 0.05 | 0.04 | 3.09 | 2.60 | 3.60 | 0.60 | 0.70 | 0.74 |
| 86 | p1E6 | 0.85 | 0.58 | 2.26 | 2.67 | 0.25 | 0.57 | 0.65 | 0.46 | 0.52 | 0.82 | 1.89 | 1.89 | 3.91 | 1.17 | 1.42 | 1.57 |
| 88 | p2B1 | 0.77 | 0.97 | 1.30 | 0.60 | 0.65 | 0.87 | 0.56 | 44.4 | 43.0 | 40.8 | 1.00 | 0.82 | 0.90 | 2.60 | 2.73 | 2.05 |
| 90 | p1D14 | 0.52 | 0.58 | 1.23 | 1.29 | 0.24 | 0.75 | 0.72 | 3.09 | 9.75 | 4.95 | 2.47 | 5.03 | 3.55 | 0.79 | 2.49 | 2.20 |
| 92 | p1D17 | 5.77 | 0.58 | 1.01 | 0.76 | 0.98 | 1.26 | 1.31 | 0.35 | 0.56 | 0.65 | 0.79 | 2.26 | 4.40 | 0.83 | 1.71 | 0.82 |
| 92 | p1P14 | 0.68 | 2.63 | 11.8 | 5.90 | 0.14 | 0.71 | 0.84 | 1.46 | 1.93 | 3.86 | 4.65 | 15.4 | 34.0 | 0.82 | 2.07 | 1.12 |
| 94 | p1C24 | 3.67 | 1.02 | 2.70 | 1.61 | 1.06 | 1.66 | 0.88 | 1.22 | 0.95 | 1.30 | 0.77 | 0.67 | 0.81 | 1.31 | 1.62 | 1.57 |
| 96 | p1D3 | 5.80 | 5.79 | 9.49 | 5.80 | 3.32 | 3.26 | 5.79 | 0.36 | 0.29 | 0.54 | 0.38 | 0.42 | 0.77 | 0.67 | 0.93 | 0.98 |
| 98 | p1E14 | 2.54 | 1.20 | 9.33 | 2.55 | 0.38 | 0.74 | 1.03 | 0.80 | 0.74 | 0.82 | 0.76 | 0.94 | 0.99 | 1.32 | 1.63 | 1.27 |
| 100 | p1E20 | 0.37 | 1.33 | 1.44 | 0.65 | 0.23 | 0.43 | 0.33 | 0.97 | 1.30 | 1.16 | 0.94 | 1.89 | 1.78 | 1.12 | 1.51 | 1.34 |
| 102 | p1A24 | 0.38 | 0.37 | 0.40 | 0.20 | 0.18 | 0.26 | 0.17 | 1.73 | 1.59 | 1.32 | 1.60 | 1.85 | 1.36 | 1.36 | 1.95 | 1.44 |
| 104 | p1E17 | 0.90 | 0.29 | 0.39 | 0.24 | 1.96 | 2.03 | 2.06 | 1.27 | 1.47 | 0.91 | 1.13 | 1.31 | 1.33 | 0.97 | 1.19 | 1.01 |
| 106 | p1E19 | 1.08 | 0.41 | 0.50 | 0.32 | 0.38 | 0.90 | 0.47 | 1.30 | 1.11 | 1.17 | 1.29 | 1.52 | 1.39 | 1.14 | 1.69 | 1.39 |
| 108 | p1E15 | 1.27 | 1.12 | 1.01 | 0.85 | 0.52 | 0.65 | 1.11 | 0.44 | 0.56 | 0.30 | 0.28 | 0.29 | 0.32 | 0.93 | 1.05 | 0.75 |
| 110 | p1E11 | 2.90 | 0.63 | 0.64 | 0.34 | 0.64 | 1.47 | 1.35 | 1.18 | 1.16 | 0.64 | 0.74 | 0.79 | 0.88 | 1.79 | 2.49 | 1.93 |
| 112 | p1E23 | 0.48 | 0.88 | 1.02 | 0.73 | 0.30 | 0.55 | 0.66 | 3.17 | 2.93 | 2.98 | 1.21 | 2.36 | 2.53 | 2.23 | 2.61 | 2.23 |
| 114 | p1E21 | 0.67 | 0.35 | 0.34 | 0.21 | 0.56 | 1.14 | 0.89 | 1.52 | 1.62 | 1.30 | 1.42 | 1.69 | 1.38 | 1.02 | 1.32 | 1.03 |
| 116 | p1D23 | 0.34 | 0.80 | 0.85 | 0.66 | 0.39 | 0.49 | 0.53 | 2.49 | 2.28 | 1.52 | 1.02 | 1.77 | 1.19 | 1.25 | 1.21 | 1.17 |
| 118 | p1D24 | 0.96 | 0.71 | 0.79 | 0.49 | 0.53 | 0.90 | 0.93 | 0.81 | 0.51 | 0.43 | 4.63 | 10.9 | 11.79 | 1.85 | 2.23 | 1.65 |
| 120 | p1D22 | 0.84 | 0.92 | 2.62 | 2.82 | 0.32 | 1.26 | 2.00 | 2.03 | 7.51 | 5.60 | 0.91 | 3.88 | 6.11 | 0.62 | 2.09 | 2.39 |
| 122 | p1E2 | 0.68 | 0.51 | 0.58 | 0.32 | 0.53 | 0.51 | 0.68 | 3.13 | 2.02 | 1.22 | 1.15 | 1.93 | 1.52 | 0.93 | 0.61 | 0.59 |
| 124 | p1E1 | 1.25 | 0.29 | 0.42 | 0.29 | 0.28 | 0.47 | 0.91 | 1.03 | 1.24 | 0.88 | 0.32 | 0.61 | 0.60 | 3.03 | 4.39 | 2.33 |
| 126 | p1E4 | 1.21 | 1.65 | 1.17 | 0.82 | 0.74 | 1.28 | 1.71 | 0.95 | 0.97 | 0.93 | 0.78 | 1.35 | 1.44 | 1.30 | 1.48 | 1.35 |
| 128 | p1D18 | 2.33 | 0.61 | 1.12 | 0.59 | 0.21 | 0.47 | 0.44 | 0.47 | 0.56 | 0.34 | 0.81 | 0.66 | 0.93 | 1.67 | 2.06 | 1.43 |
| 130 | p1D21 | 1.58 | 0.75 | 0.63 | 0.26 | 0.63 | 0.94 | 1.35 | 1.42 | 1.59 | 1.61 | 4.36 | 8.21 | 7.50 | 0.98 | 1.29 | 1.38 |
| 132 | p1C22 | 3.39 | 0.24 | 0.24 | 0.21 | 1.34 | 1.44 | 2.22 | 0.54 | 0.74 | 0.36 | 0.34 | 0.38 | 0.45 | 0.93 | 1.16 | 0.89 |
| 134 | p1C23 | 0.85 | 0.87 | 1.53 | 0.69 | 0.31 | 0.63 | 0.59 | 1.02 | 0.68 | 1.28 | 1.64 | 2.00 | 2.74 | 1.27 | 1.80 | 1.29 |
| 136 | p1D11 | 0.90 | 0.33 | 2.31 | 1.23 | 0.26 | 1.00 | 1.34 | 0.38 | 0.41 | 1.04 | 0.49 | 2.07 | 2.79 | 0.86 | 1.77 | 1.60 |
| 138 | p1E3 | 3.29 | 0.99 | 0.43 | 0.18 | 5.87 | 6.74 | 7.79 | 0.35 | 0.44 | 0.17 | 1.79 | 0.50 | 0.31 | 2.09 | 1.61 | 1.29 |
| 140 | p1D20 | 0.78 | 1.34 | 1.76 | 0.65 | 0.34 | 0.71 | 0.72 | 2.43 | 2.48 | 1.46 | 0.66 | 1.80 | 1.75 | 3.27 | 3.69 | 4.17 |
| 142 | p1E5 | 3.83 | 0.83 | 1.03 | 0.57 | 0.83 | 1.57 | 1.37 | 0.67 | 0.70 | 0.68 | 0.55 | 0.64 | 0.78 | 1.19 | 1.55 | 1.19 |
| 144 | p1D19 | 1.97 | 0.16 | 0.12 | 0.14 | 0.80 | 1.04 | 1.08 | 0.46 | 0.58 | 0.25 | 0.49 | 0.46 | 0.59 | 1.34 | 1.62 | 1.22 |
| 146 | p2A15 | 0.54 | 0.64 | 0.82 | 0.41 | 0.64 | 0.39 | 0.40 | 1.61 | 1.48 | 1.44 | 2.08 | 2.36 | 2.45 | 1.01 | 1.17 | 1.02 |
| 148 | p1I14 | 0.26 | 2.20 | 3.06 | 1.28 | 0.15 | 0.27 | 0.19 | 1.67 | 1.28 | 0.67 | 1.87 | 2.43 | 1.77 | 1.06 | 1.00 | 0.68 |
| 150 | p1I2 | 0.93 | 1.17 | 1.93 | 0.88 | 0.16 | 0.30 | 0.31 | 1.14 | 1.13 | 1.01 | 0.36 | 0.55 | 0.54 | 3.57 | 5.13 | 2.75 |
| 152 | p1I12 | 2.71 | 0.54 | 0.27 | 0.32 | 2.90 | 3.25 | 2.42 | 0.62 | 0.85 | 0.44 | 0.36 | 0.34 | 0.40 | 0.87 | 1.00 | 0.87 |
| 154 | p1I3 | 0.76 | 0.94 | 1.64 | 0.59 | 0.45 | 0.72 | 0.52 | 1.46 | 1.20 | 0.61 | 0.88 | 0.93 | 0.81 | 1.46 | 1.81 | 1.11 |
| 156 | p1I10 | 0.85 | 1.50 | 1.17 | 0.73 | 0.92 | 0.90 | 1.14 | 4.02 | 4.49 | 4.46 | 0.33 | 0.26 | 0.34 | 1.22 | 1.28 | 0.89 |
| 158 | p1H18 | 0.22 | 1.07 | 0.49 | 0.15 | 0.65 | 0.80 | 1.00 | 1.71 | 1.19 | 0.75 | 1.47 | 0.95 | 0.79 | 1.78 | 1.45 | 1.00 |
| 160 | p1H24 | 4.74 | 0.93 | 0.84 | 0.50 | 0.76 | 0.89 | 1.07 | 1.06 | 0.87 | 0.78 | 0.90 | 0.62 | 0.50 | 1.24 | 1.24 | 0.87 |
| 162 | p1E22 | 1.62 | 1.04 | 1.05 | 0.62 | 0.85 | 1.66 | 1.16 | 1.07 | 1.02 | 0.90 | 1.01 | 1.27 | 1.27 | 1.05 | 1.39 | 0.89 |
| 164 | p1H21 | 12.1 | 0.34 | 0.94 | 0.49 | 0.32 | 0.82 | 2.11 | 0.63 | 1.14 | 1.03 | 0.80 | 1.43 | 2.23 | 1.28 | 1.97 | 1.15 |
| 166 | p1I1 | 1.37 | 0.52 | 0.45 | 0.30 | 1.22 | 1.28 | 1.73 | 0.98 | 0.70 | 0.51 | 0.66 | 0.51 | 0.77 | 2.16 | 2.43 | 1.44 |
| 168 | p1H14 | 0.35 | 1.14 | 1.18 | 1.02 | 0.30 | 0.42 | 0.37 | 3.00 | 3.19 | 1.59 | 1.71 | 2.48 | 2.09 | 1.35 | 1.56 | 0.99 |
| 170 | p1H11 | 0.74 | 0.24 | 0.41 | 0.22 | 0.42 | 0.68 | 0.92 | 1.28 | 1.28 | 1.26 | 2.20 | 2.56 | 1.81 | 1.12 | 1.13 | 0.97 |
| 172 | p1H17 | 0.24 | 1.28 | 0.58 | 0.15 | 0.77 | 0.80 | 0.85 | 1.58 | 0.98 | 0.67 | 1.43 | 1.10 | 0.82 | 1.49 | 1.02 | 0.71 |
| 174 | p1H12 | 1.03 | 1.02 | 1.56 | 0.71 | 0.58 | 0.95 | 0.61 | 0.93 | 1.28 | 1.16 | 0.51 | 0.63 | 0.76 | 2.85 | 3.33 | 2.57 |
| 176 | p1H7 | 0.31 | 0.35 | 0.20 | 0.11 | 0.54 | 0.71 | 0.76 | 1.81 | 1.40 | 1.00 | 1.11 | 0.77 | 0.96 | 2.27 | 1.45 | 1.10 |
| 178 | p1H15 | 2.35 | 0.61 | 0.32 | 0.18 | 1.05 | 1.30 | 1.88 | 1.00 | 0.73 | 0.43 | 0.83 | 0.48 | 0.37 | 1.77 | 1.51 | 0.96 |
| 180 | p1H20 | 0.25 | 1.42 | 0.56 | 0.18 | 0.72 | 0.91 | 0.93 | 1.69 | 1.41 | 1.01 | 1.29 | 0.91 | 0.81 | 2.25 | 1.41 | 1.08 |
| 182 | p1H8 | 0.42 | 0.15 | 0.06 | 0.03 | 0.94 | 0.98 | 1.07 | 0.87 | 0.57 | 0.32 | 0.78 | 0.44 | 0.67 | 2.28 | 1.78 | 1.17 |
| 184 | p1H16 | 0.76 | 0.85 | 0.99 | 0.58 | 0.93 | 1.24 | 0.88 | 1.55 | 1.06 | 1.70 | 0.63 | 0.51 | 1.04 | 1.51 | 1.13 | 1.21 |
| 186 | p1H9 | 0.47 | 1.26 | 1.82 | 1.00 | 0.38 | 0.46 | 0.73 | 3.02 | 1.86 | 2.13 | 2.67 | 3.21 | 2.86 | 1.66 | 1.28 | 0.87 |
| 188 | p1H23 | 0.38 | 1.16 | 1.05 | 0.99 | 0.38 | 0.46 | 0.53 | 4.59 | 3.73 | 1.67 | 1.90 | 4.02 | 2.27 | 1.30 | 1.08 | 0.77 |
| 190 | p1H10 | 3.68 | 0.65 | 0.65 | 0.43 | 0.69 | 0.81 | 1.24 | 0.73 | 0.80 | 0.71 | 0.35 | 0.39 | 0.50 | 1.91 | 1.81 | 2.21 |
| 192 | p1H6 | 6.08 | 0.16 | 0.13 | 0.10 | 1.87 | 1.88 | 2.03 | 0.17 | 0.25 | 0.13 | 0.13 | 0.10 | 0.12 | 0.85 | 1.17 | 0.85 |
| 194 | p1H13 | 0.72 | 4.92 | 3.05 | 0.37 | 2.06 | 1.40 | 1.91 | 0.51 | 0.46 | 0.90 | 0.68 | 0.37 | 0.49 | 1.05 | 0.47 | 0.87 |
| 196 | p1H19 | 0.80 | 2.90 | 2.66 | 0.49 | 0.59 | 0.89 | 1.56 | 0.62 | 0.44 | 0.82 | 2.07 | 1.40 | 0.59 | 0.86 | 1.61 | 0.95 |
| 198 | p1G22 | 0.77 | 0.58 | 0.60 | 0.24 | 0.33 | 0.47 | 0.49 | 0.94 | 0.72 | 1.03 | 1.65 | 1.71 | 1.62 | 1.88 | 2.51 | 2.06 |
| 200 | p1G21 | 0.74 | 1.03 | 1.34 | 0.49 | 0.38 | 0.57 | 0.73 | 0.92 | 0.65 | 1.04 | 1.47 | 1.29 | 1.15 | 1.20 | 1.47 | 1.03 |
| 202 | p1H1 | 1.08 | 0.46 | 0.38 | 0.25 | 0.65 | 0.94 | 1.02 | 1.44 | 1.84 | 1.44 | 1.77 | 1.69 | 1.29 | 1.01 | 0.97 | 0.81 |
| 204 | p1G20 | 1.09 | 1.85 | 1.50 | 0.64 | 1.27 | 1.46 | 1.03 | 0.85 | 0.67 | 0.37 | 1.51 | 1.05 | 0.70 | 1.01 | 1.11 | 0.85 |
| 206 | p1H5 | 1.14 | 0.49 | 0.58 | 0.40 | 0.69 | 0.78 | 0.91 | 1.07 | 0.84 | 1.04 | 0.55 | 0.44 | 0.29 | 2.09 | 2.45 | 2.06 |
| 208 | p1G19 | 0.29 | 2.03 | 1.39 | 0.36 | 0.48 | 0.51 | 0.35 | 1.14 | 0.90 | 1.76 | 1.78 | 1.49 | 2.25 | 1.00 | 1.01 | 0.56 |
| 210 | p1H2 | 1.86 | 0.91 | 0.82 | 0.72 | 1.94 | 2.47 | 2.65 | 2.55 | 2.29 | 0.35 | 1.66 | 2.25 | 1.06 | 0.61 | 0.57 | 0.36 |
| 212 | p1G18 | 0.90 | 3.11 | 3.14 | 0.50 | 1.10 | 1.18 | 1.51 | 0.54 | 0.81 | 1.17 | 1.05 | 0.84 | 1.08 | 0.79 | 1.11 | 0.74 |
| 214 | p1H4 | 1.26 | 0.21 | 0.08 | 0.04 | 1.06 | 1.04 | 1.37 | 0.81 | 0.78 | 0.44 | 0.69 | 0.54 | 0.45 | 1.26 | 0.89 | 0.74 |
| 216 | p1H3 | 2.12 | 0.84 | 0.35 | 0.14 | 1.34 | 1.57 | 2.53 | 1.32 | 0.67 | 0.44 | 1.11 | 0.48 | 0.44 | 2.21 | 1.52 | 0.90 |
| 222 | p1P3 | 1.92 | 0.44 | 0.79 | 0.38 | 0.27 | 0.44 | 0.66 | 0.47 | 0.55 | 0.30 | 7.15 | 7.93 | 6.97 | 1.12 | 1.18 | 1.12 |
| 224 | p1A9 | 0.79 | 1.08 | 2.70 | 3.21 | 0.06 | 0.35 | 0.47 | 0.16 | 0.20 | 1.42 | 0.79 | 1.64 | 3.82 | 0.72 | 1.78 | 3.33 |
| 224 | p1A8 | 6.46 | 4.52 | 37.2 | 24.2 | 0.63 | 1.17 | 1.40 | 0.13 | 0.14 | 0.44 | 0.27 | 0.49 | 1.37 | 0.39 | 0.56 | 1.59 |
| 226 | p1B17 | 6.59 | 27.2 | 223 | 109 | 0.51 | 1.00 | 0.68 | 0.81 | 0.85 | 0.86 | 0.83 | 1.22 | 2.43 | 0.86 | 1.55 | 1.09 |
| 228 | p1O20 | 0.27 | 4.25 | 7.39 | 5.74 | 0.09 | 0.44 | 0.44 | 1.19 | 14.2 | 31.1 | 5.43 | 8.1 | 15.1 | 0.68 | 2.46 | 5.18 |
| 230 | p1B2 | 0.99 | 7.01 | 22.2 | 28.8 | 0.33 | 0.98 | 1.70 | 0.44 | 0.54 | 2.32 | 0.67 | 4.52 | 13.0 | 0.51 | 1.10 | 1.44 |
| 232 | p1B3 | 0.85 | 0.33 | 1.27 | 1.04 | 0.09 | 0.42 | 1.08 | 0.33 | 0.41 | 3.73 | 0.97 | 1.72 | 1.54 | 0.84 | 1.63 | 2.88 |
| 236 | p1B19 | 0.07 | 0.36 | 2.23 | 1.77 | 0.01 | 0.03 | 0.06 | 0.01 | 0.01 | 0.01 | 1.23 | 0.69 | 3.23 | 0.80 | 2.03 | 1.54 |
| 236 | p1B18 | 0.89 | 2.35 | 29.2 | 13.7 | 0.20 | 0.34 | 0.39 | 0.17 | 0.17 | 0.15 | 0.21 | 0.17 | 0.69 | 1.36 | 0.66 | 1.75 |
| 238 | p1N17 | 1.67 | 3.14 | 3.81 | 8.23 | 0.41 | 0.43 | 0.63 | 0.35 | 0.37 | 0.23 | 0.16 | 0.18 | 0.21 | 0.71 | 0.86 | 1.73 |
| 240 | p1A24 | 3.26 | 0.37 | 1.01 | 0.74 | 0.44 | 0.77 | 1.17 | 0.10 | 0.13 | 0.08 | 1.85 | 0.91 | 2.15 | 0.66 | 1.03 | 0.82 |
| 244 | p1B1 | 1.42 | 0.65 | 2.03 | 1.31 | 0.01 | 0.74 | 1.05 | 0.03 | 0.02 | 0.04 | 2.53 | 1.72 | 2.76 | 0.61 | 1.04 | 0.88 |
| 248 | p1A4 | 0.84 | 0.22 | 1.78 | 1.26 | 0.31 | 0.89 | 0.94 | 1.14 | 1.97 | 0.82 | 2.22 | 2.17 | 3.25 | 1.70 | 2.50 | 2.14 |
| 248 | p1A2 | 3.47 | 0.51 | 12.1 | 8.87 | 0.33 | 2.30 | 2.64 | 0.86 | 1.50 | 0.70 | 0.91 | 1.32 | 2.58 | 0.58 | 0.80 | 0.86 |
| 248 | p1A3 | 5.15 | 1.31 | 31.9 | 18.8 | 0.41 | 4.16 | 5.14 | 1.20 | 1.46 | 0.63 | 0.63 | 1.50 | 2.78 | 0.52 | 0.97 | 1.02 |
| 248 | p1A1 | 10.0 | 1.02 | 49.3 | 22.8 | 0.76 | 4.24 | 4.31 | 0.75 | 0.62 | 0.72 | 0.53 | 0.70 | 2.19 | 0.87 | 0.94 | 1.07 |
| 250 | p1A16 | 5.53 | 0.80 | 9.87 | 6.89 | 0.39 | 1.20 | 1.04 | 0.55 | 0.56 | 1.34 | 0.58 | 0.85 | 3.22 | 0.91 | 1.02 | 1.08 |
| 250 | p1A15 | 6.09 | 0.90 | 5.67 | 6.24 | 0.29 | 2.68 | 3.57 | 0.83 | 2.06 | 13.2 | 0.73 | 2.53 | 7.79 | 0.18 | 1.20 | 1.04 |
| 250 | p1A18 | 4.95 | 1.01 | 4.25 | 7.77 | 0.50 | 2.95 | 4.85 | 2.05 | 8.73 | 12.21 | 1.02 | 2.75 | 7.82 | 0.31 | 0.84 | 0.85 |
| 250 | p1A17 | 22.0 | 1.16 | 34.1 | 13.81 | 1.85 | 4.66 | 2.89 | 1.18 | 0.95 | 0.90 | 0.76 | 1.10 | 2.00 | 1.02 | 1.40 | 1.08 |
| 252 | p1B14 | 0.22 | 0.45 | 0.65 | 1.54 | 0.84 | 6.03 | 2.44 | 0.04 | 0.05 | 0.05 | 2.81 | 1.35 | 4.13 | 0.30 | 0.29 | 0.30 |
| 252 | p1B15 | 0.24 | 0.34 | 0.46 | 0.83 | 0.54 | 4.88 | 2.25 | 0.05 | 0.05 | 0.06 | 8.90 | 3.01 | 8.69 | 1.01 | 0.68 | 0.91 |
| 252 | p1B16 | 0.24 | 0.47 | 0.45 | 1.35 | 0.78 | 5.19 | 2.01 | 0.05 | 0.04 | 0.06 | 8.93 | 3.67 | 13.9 | 0.95 | 0.67 | 0.82 |
| 254 | p1A12 | 1.06 | 2.50 | 3.37 | 4.51 | 0.19 | 0.38 | 1.19 | 0.37 | 0.63 | 2.83 | 0.92 | 2.17 | 3.37 | 0.60 | 1.03 | 1.69 |
| 254 | p1A11 | 26.9 | 6.17 | 142 | 62.4 | 2.33 | 5.10 | 6.21 | 0.13 | 0.22 | 0.81 | 0.17 | 0.44 | 1.01 | 0.53 | 0.29 | 1.22 |
| 256 | p1A13 | 3.13 | 1.49 | 4.12 | 4.95 | 0.92 | 1.18 | 2.38 | 0.06 | 0.06 | 1.27 | 0.29 | 0.81 | 2.00 | 0.21 | 0.36 | 1.05 |
| 258 | p1A14 | 7.75 | 6.29 | 28.0 | 29.3 | 4.13 | 5.41 | 7.32 | 0.17 | 0.24 | 0.33 | 0.11 | 0.23 | 0.77 | 0.59 | 0.39 | 0.89 |
| 260 | p1A19 | 15.7 | 1.21 | 33.7 | 4.98 | 0.74 | 5.11 | 2.30 | 0.68 | 0.81 | 0.61 | 0.43 | 0.56 | 0.52 | 0.86 | 0.64 | 0.89 |
| 262 | p1A20 | 4.93 | 3.33 | 10.0 | 7.74 | 0.98 | 1.70 | 2.70 | 0.08 | 0.08 | 0.12 | 0.08 | 0.14 | 0.57 | 0.38 | 0.18 | 1.26 |
| 264 | p1A22 | 0.87 | 0.84 | 1.41 | 1.18 | 0.07 | 0.24 | 0.32 | 0.61 | 1.92 | 3.26 | 2.03 | 4.60 | 4.51 | 0.75 | 2.15 | 3.17 |
| 266 | p1A23 | 1.14 | 1.34 | 4.67 | 2.78 | 0.02 | 0.79 | 1.41 | 0.01 | 0.01 | 0.01 | 1.17 | 0.96 | 2.10 | 0.13 | 0.23 | 0.21 |
| 268 | p1B21 | 165 | 0.17 | 0.31 | 0.17 | 1.35 | 3.24 | 55.7 | 0.37 | 0.40 | 0.27 | 28.9 | 30.2 | 86.8 | 0.90 | 1.00 | 0.67 |
| 268 | p1B20 | 2952 | 1.05 | 3.07 | 1.20 | 17.5 | 32.8 | 682 | 0.76 | 0.85 | 1.29 | 45.1 | 42.8 | 224 | 1.14 | 0.96 | 1.15 |
| 270 | p1C17 | 5.82 | 3.02 | 5.66 | 2.13 | 4.54 | 4.06 | 4.88 | 0.46 | 0.55 | 0.59 | 0.80 | 0.95 | 1.70 | 0.92 | 0.83 | 1.03 |
| 270 | p1C18 | 8.06 | 2.62 | 7.14 | 2.17 | 3.71 | 3.18 | 4.70 | 0.45 | 0.66 | 0.69 | 0.67 | 1.03 | 1.59 | 0.91 | 1.00 | 0.96 |
| 272 | p1D8 | 2.31 | 0.09 | 0.17 | 0.18 | 1.11 | 1.09 | 1.50 | 0.62 | 0.97 | 0.73 | 0.27 | 0.44 | 0.80 | 1.16 | 1.31 | 1.07 |
| 274 | p1A10 | 3.43 | 0.14 | 2.68 | 4.91 | 0.12 | 0.78 | 1.90 | 0.53 | 0.36 | 6.58 | 0.90 | 5.24 | 22.3 | 3.10 | 0.91 | 2.13 |
| 276 | p1G24 | 1.02 | 0.42 | 1.73 | 0.82 | 0.17 | 0.45 | 0.48 | 0.62 | 1.04 | 1.26 | 0.48 | 1.02 | 1.36 | 2.79 | 4.68 | 4.49 |
| 278 | p1G23 | 2.13 | 0.90 | 2.33 | 0.80 | 0.73 | 1.13 | 1.75 | 0.66 | 0.90 | 0.81 | 0.44 | 0.84 | 0.95 | 1.72 | 2.05 | 1.83 |
| 280 | p1G5 | 0.90 | 1.21 | 3.69 | 3.47 | 0.45 | 1.28 | 2.11 | 0.89 | 2.10 | 4.01 | 0.68 | 2.58 | 5.12 | 0.36 | 1.31 | 1.57 |
| 282 | p1G7 | 0.52 | 1.12 | 1.85 | 1.19 | 0.23 | 0.40 | 0.50 | 1.27 | 0.92 | 1.35 | 1.62 | 2.39 | 1.90 | 0.94 | 1.54 | 1.32 |
| 284 | p2A23 | 2.36 | 0.59 | 0.61 | 0.40 | 0.32 | 0.40 | 0.35 | 1.32 | 1.14 | 0.79 | 0.85 | 0.76 | 0.66 | 1.68 | 2.18 | 1.53 |
| 286 | p1G1 | 0.35 | 0.53 | 0.85 | 0.75 | 1.04 | 1.89 | 1.39 | 1.17 | 1.16 | 0.98 | 0.74 | 0.91 | 0.95 | 1.16 | 1.20 | 1.52 |
| 288 | p1G15 | 4.87 | 0.34 | 0.56 | 0.36 | 1.92 | 1.93 | 1.93 | 0.33 | 0.63 | 0.51 | 0.20 | 0.28 | 0.32 | 0.80 | 1.05 | 0.89 |
| 290 | p1F23 | 0.32 | 0.84 | 0.77 | 0.70 | 0.29 | 0.32 | 0.32 | 1.50 | 1.79 | 1.29 | 2.59 | 2.59 | 1.90 | 1.67 | 1.38 | 1.16 |
| 292 | p1G8 | 0.43 | 0.49 | 0.79 | 0.86 | 0.50 | 0.62 | 0.52 | 13.4 | 14.8 | 10.4 | 1.32 | 4.10 | 3.54 | 0.96 | 2.78 | 2.43 |
| 294 | p1G13 | 9.42 | 0.46 | 0.47 | 0.43 | 2.39 | 2.87 | 3.25 | 0.15 | 0.16 | 0.13 | 0.28 | 0.43 | 0.64 | 0.79 | 1.19 | 0.96 |
| 296 | p1G10 | 0.49 | 0.67 | 0.68 | 0.40 | 0.30 | 0.44 | 0.37 | 1.46 | 0.96 | 0.72 | 2.48 | 2.48 | 1.88 | 1.56 | 1.51 | 1.21 |
| 298 | p1F24 | 0.08 | 2.07 | 1.62 | 1.89 | 0.13 | 0.17 | 0.19 | 0.45 | 0.58 | 0.36 | 1.18 | 1.06 | 1.55 | 6.29 | 6.20 | 4.26 |
| 300 | p1G2 | 0.62 | 0.61 | 0.84 | 0.43 | 0.28 | 0.29 | 0.48 | 0.89 | 0.65 | 1.21 | 0.91 | 0.92 | 0.69 | 2.10 | 2.64 | 2.62 |
| 302 | p1G11 | 0.65 | 3.60 | 4.27 | 4.45 | 0.16 | 0.26 | 0.20 | 5.96 | 8.65 | 5.54 | 11.3 | 18.7 | 26.3 | 0.98 | 1.30 | 1.18 |
| 304 | p1G16 | 0.36 | 0.71 | 1.07 | 0.87 | 0.87 | 1.07 | 1.38 | 8.25 | 8.61 | 5.74 | 0.97 | 2.34 | 3.68 | 1.07 | 1.21 | 1.14 |
| 306 | p1G9 | 1.43 | 1.28 | 1.06 | 1.00 | 0.49 | 1.14 | 1.76 | 0.85 | 0.66 | 0.52 | 1.39 | 1.80 | 2.13 | 0.98 | 1.04 | 0.96 |
| 308 | p1G4 | 2.65 | 0.95 | 1.70 | 1.60 | 0.56 | 0.77 | 0.78 | 2.02 | 0.93 | 0.45 | 1.44 | 3.73 | 5.27 | 4.99 | 7.58 | 9.71 |
| 310 | p1G14 | 4.68 | 0.15 | 0.16 | 0.13 | 1.08 | 1.48 | 1.63 | 0.33 | 0.49 | 0.25 | 0.17 | 0.18 | 0.18 | 0.94 | 1.57 | 1.09 |
| 312 | p1A6 | 1.29 | 0.55 | 2.17 | 2.92 | 0.15 | 0.91 | 1.99 | 0.99 | 2.21 | 1.94 | 1.18 | 3.56 | 5.14 | 0.40 | 1.08 | 1.87 |
| 312 | p1A5 | 3.93 | 0.19 | 0.23 | 0.22 | 0.98 | 1.78 | 3.76 | 0.25 | 0.33 | 0.24 | 0.15 | 0.20 | 0.26 | 1.25 | 1.65 | 1.39 |
| 314 | p1B9 | 4.45 | 0.17 | 0.47 | 0.17 | 5.52 | 18.9 | 19.0 | 0.16 | 0.16 | 0.41 | 0.29 | 0.57 | 4.92 | 0.32 | 0.61 | 1.23 |
| 314 | p1B6 | 8.12 | 1.22 | 2.38 | 1.81 | 10.0 | 25.1 | 16.0 | 0.60 | 0.57 | 0.58 | 0.99 | 1.38 | 2.38 | 0.79 | 0.54 | 0.87 |
| 314 | p1B8 | 7.34 | 0.64 | 1.60 | 1.29 | 5.86 | 21.3 | 21.4 | 0.55 | 0.47 | 0.46 | 0.39 | 0.44 | 1.19 | 0.43 | 0.55 | 1.38 |
| 314 | p1B7 | 12.00 | 1.15 | 2.78 | 1.89 | 21.1 | 41.1 | 42.4 | 0.46 | 0.44 | 0.57 | 0.75 | 0.85 | 1.97 | 0.46 | 0.40 | 0.97 |
| 316 | p1G17 | 0.93 | 1.06 | 1.46 | 0.76 | 0.24 | 0.44 | 0.99 | 1.10 | 0.77 | 0.82 | 2.00 | 2.84 | 3.17 | 0.96 | 0.97 | 1.00 |
| 318 | p1G3 | 0.30 | 1.65 | 1.68 | 1.50 | 0.58 | 0.86 | 1.02 | 0.95 | 0.99 | 1.52 | 1.23 | 1.74 | 3.24 | 1.28 | 0.97 | 1.78 |
| 320 | p1F22 | 0.35 | 0.74 | 0.85 | 0.83 | 0.16 | 0.27 | 0.31 | 1.51 | 1.59 | 0.85 | 2.54 | 3.06 | 3.72 | 1.03 | 1.01 | 0.75 |
| 322 | p1G12 | 0.39 | 1.53 | 2.64 | 2.85 | 0.33 | 0.51 | 0.84 | 1.69 | 3.85 | 4.80 | 0.83 | 3.71 | 5.25 | 1.74 | 2.60 | 3.23 |
| 324 | p1F11 | 0.89 | 0.64 | 0.85 | 0.57 | 0.98 | 1.77 | 1.64 | 2.48 | 2.83 | 1.47 | 0.46 | 0.74 | 0.73 | 1.62 | 1.80 | 1.81 |
| 326 | p1F16 | 3.38 | 1.02 | 0.52 | 0.21 | 7.19 | 6.39 | 9.11 | 0.31 | 0.33 | 0.16 | 1.44 | 0.39 | 0.29 | 1.68 | 1.44 | 1.20 |
| 328 | p1F14 | 0.30 | 1.52 | 3.98 | 3.61 | 1.00 | 1.72 | 1.66 | 0.06 | 0.07 | 0.07 | 3.27 | 4.66 | 10.6 | 1.25 | 1.05 | 1.46 |
| 330 | p1F17 | 1.32 | 11.7 | 16.0 | 9.79 | 0.04 | 0.27 | 1.01 | 0.06 | 0.07 | 0.07 | 0.12 | 0.12 | 0.23 | 1.19 | 2.33 | 1.66 |
| 332 | p1C2 | 2.00 | 0.60 | 2.84 | 1.53 | 7.05 | 9.91 | 9.48 | 0.95 | 18.0 | 39.5 | 0.27 | 1.57 | 3.32 | 0.52 | 0.80 | 0.64 |
| 334 | p1F3 | 0.87 | 0.56 | 0.75 | 0.45 | 0.40 | 0.64 | 0.66 | 1.14 | 0.99 | 0.74 | 0.79 | 1.38 | 1.14 | 1.82 | 2.15 | 1.65 |
| 336 | p1F20 | 0.98 | 2.60 | 4.08 | 2.62 | 1.11 | 1.52 | 2.07 | 0.27 | 0.27 | 0.71 | 0.37 | 0.71 | 1.14 | 0.70 | 0.89 | 1.08 |
| 338 | p1F6 | 0.91 | 0.45 | 1.45 | 0.91 | 0.01 | 0.45 | 0.61 | 0.04 | 0.05 | 0.04 | 2.18 | 2.32 | 3.68 | 0.92 | 1.03 | 0.84 |
| 340 | p1F4 | 5.14 | 2.83 | 2.10 | 1.95 | 0.44 | 0.65 | 0.99 | 0.87 | 0.89 | 0.78 | 0.73 | 0.62 | 0.82 | 1.34 | 1.50 | 1.15 |
| 342 | p1F15 | 1.06 | 0.79 | 1.22 | 0.71 | 0.35 | 0.56 | 0.84 | 1.57 | 1.93 | 0.90 | 1.16 | 1.46 | 2.33 | 1.36 | 1.38 | 0.93 |
| 344 | p1F13 | 0.71 | 0.43 | 1.58 | 1.80 | 0.28 | 1.94 | 2.63 | 2.51 | 14.4 | 12.9 | 0.82 | 6.59 | 7.42 | 1.07 | 3.89 | 3.99 |
| 346 | p1A7 | 2.67 | 5.16 | 9.61 | 4.32 | 1.15 | 2.12 | 2.18 | 0.99 | 0.37 | 0.61 | 1.58 | 1.30 | 1.60 | 1.86 | 2.36 | 1.59 |
| 348 | p1A21 | 0.24 | 0.86 | 1.13 | 1.08 | 0.18 | 0.26 | 0.36 | 0.61 | 0.33 | 0.38 | 1.19 | 1.97 | 1.36 | 2.27 | 2.95 | 3.16 |
| 350 | p1B5 | 0.10 | 0.90 | 1.37 | 1.04 | 0.02 | 0.03 | 0.06 | 0.18 | 0.30 | 1.85 | 1.47 | 1.82 | 3.02 | 1.66 | 3.19 | 3.40 |
| 350 | p1B4 | 0.87 | 3.69 | 9.15 | 4.61 | 0.25 | 0.22 | 0.48 | 0.32 | 0.30 | 0.80 | 0.52 | 0.79 | 2.38 | 1.93 | 1.75 | 5.01 |
| 352 | p1B12 | 1.82 | 0.74 | 0.84 | 1.09 | 0.75 | 1.02 | 0.97 | 0.49 | 0.64 | 0.37 | 1.28 | 1.90 | 1.56 | 1.10 | 1.37 | 1.41 |
| 352 | p1B11 | 1.45 | 1.42 | 2.25 | 1.85 | 0.06 | 0.10 | 0.22 | 0.93 | 1.11 | 1.39 | 2.23 | 3.90 | 3.56 | 2.04 | 2.69 | 3.58 |
| 352 | p1B10 | 3.03 | 1.34 | 6.67 | 4.03 | 1.05 | 1.41 | 1.44 | 0.72 | 0.82 | 0.98 | 0.55 | 0.67 | 0.88 | 0.90 | 1.41 | 1.05 |
| 354 | p1B13 | 1.66 | 1.24 | 1.26 | 0.83 | 1.96 | 1.86 | 2.20 | 0.55 | 0.76 | 0.41 | 0.31 | 0.29 | 0.39 | 0.98 | 0.94 | 1.04 |
| 356 | p1B22 | 0.29 | 2.78 | 2.80 | 2.50 | 0.05 | 0.06 | 0.07 | 0.13 | 0.11 | 0.04 | 0.26 | 0.87 | 0.39 | 3.93 | 3.62 | 4.04 |
| 358 | p1B23 | 8.51 | 1.84 | 6.94 | 6.08 | 1.79 | 2.46 | 3.57 | 0.49 | 0.59 | 0.97 | 0.52 | 1.43 | 4.14 | 0.58 | 0.86 | 0.83 |
| 360 | p1B24 | 1.27 | 1.32 | 1.41 | 1.33 | 0.92 | 1.14 | 1.14 | 4.95 | 2.46 | 1.92 | 2.93 | 3.67 | 5.83 | 0.80 | 0.98 | 0.76 |
| 362 | p1C3 | 0.55 | 1.86 | 2.73 | 1.77 | 0.73 | 1.09 | 1.29 | 0.18 | 0.10 | 0.36 | 1.15 | 1.00 | 1.09 | 1.01 | 2.33 | 1.40 |
| 364 | p1C4 | 1.78 | 0.92 | 6.16 | 1.47 | 0.55 | 0.81 | 0.71 | 1.06 | 0.88 | 0.96 | 0.67 | 0.66 | 0.78 | 1.44 | 2.81 | 1.64 |
| 366 | p1C5 | 0.89 | 0.72 | 1.39 | 1.15 | 0.39 | 0.96 | 1.04 | 1.46 | 1.37 | 1.15 | 1.58 | 3.45 | 4.04 | 0.73 | 1.21 | 0.77 |
| 368 | p1C6 | 19.92 | 1.79 | 28.4 | 14.2 | 2.36 | 5.29 | 5.01 | 0.57 | 0.94 | 1.90 | 0.38 | 0.60 | 1.00 | 0.57 | 0.66 | 0.96 |
| 370 | p1C7 | 1.69 | 2.98 | 11.76 | 3.31 | 1.90 | 1.61 | 1.45 | 0.68 | 0.64 | 0.58 | 0.51 | 0.85 | 0.76 | 0.84 | 1.00 | 0.74 |
| 372 | p1C8 | 1.04 | 1.70 | 7.73 | 8.58 | 0.20 | 0.66 | 0.98 | 0.04 | 0.54 | 3.98 | 3.29 | 9.13 | 21.3 | 0.23 | 1.77 | 1.66 |
| 374 | p1C9 | 1.00 | 0.81 | 1.62 | 1.21 | 0.84 | 0.80 | 1.01 | 0.46 | 0.38 | 0.49 | 0.92 | 1.27 | 1.68 | 1.15 | 1.33 | 1.56 |
| 376 | p1C10 | 28.7 | 0.64 | 1.66 | 0.81 | 1.18 | 2.19 | 2.92 | 0.89 | 1.18 | 1.55 | 0.76 | 0.77 | 1.09 | 0.89 | 0.94 | 0.70 |
| 378 | p1C11 | 1.45 | 1.73 | 11.0 | 5.75 | 0.58 | 0.87 | 0.78 | 0.13 | 0.07 | 0.56 | 0.81 | 1.46 | 3.60 | 1.00 | 1.63 | 1.11 |
| 380 | p1C12 | 3.79 | 0.76 | 24.4 | 7.12 | 0.84 | 1.64 | 0.79 | 0.86 | 0.91 | 0.84 | 0.76 | 0.97 | 1.41 | 1.05 | 0.81 | 1.04 |
| 382 | p1C13 | 2.43 | 0.98 | 2.13 | 1.13 | 1.32 | 2.88 | 3.71 | 0.33 | 0.41 | 0.60 | 0.37 | 0.55 | 0.94 | 1.14 | 0.95 | 2.34 |
| 384 | p1C14 | 2.68 | 2.62 | 7.82 | 2.55 | 1.95 | 1.59 | 1.62 | 0.47 | 0.44 | 0.39 | 0.35 | 0.45 | 1.13 | 0.50 | 0.76 | 0.79 |
| 386 | p1C15 | 0.66 | 0.61 | 0.97 | 0.72 | 0.47 | 0.73 | 0.78 | 30.7 | 28.4 | 10.3 | 2.22 | 5.85 | 6.44 | 1.82 | 1.79 | 1.18 |
| 388 | p1C16 | 1.76 | 0.94 | 25.0 | 14.8 | 0.37 | 0.41 | 0.59 | 0.42 | 0.61 | 0.33 | 0.35 | 0.67 | 1.30 | 0.63 | 1.28 | 0.97 |
| 390 | p1C19 | 26.3 | 4.16 | 5.72 | 3.15 | 1.55 | 1.72 | 4.66 | 0.72 | 0.74 | 0.80 | 0.76 | 1.04 | 1.09 | 0.50 | 0.61 | 0.65 |
| 392 | p1C20 | 0.55 | 0.81 | 1.58 | 1.69 | 0.51 | 0.82 | 1.21 | 0.51 | 0.37 | 1.45 | 1.10 | 3.34 | 4.67 | 0.67 | 2.21 | 1.87 |
| 396 | p1P5 | 0.53 | 0.17 | 0.23 | 0.12 | 0.94 | 0.49 | 0.27 | 0.53 | 0.47 | 0.33 | 6.09 | 2.15 | 4.70 | 0.79 | 0.78 | 0.59 |
| 398 | p2L23 | 0.29 | 2.63 | 1.23 | 1.15 | 0.22 | 0.31 | 0.34 | 0.77 | 0.70 | 0.55 | 28.3 | 15.9 | 28.5 | 2.59 | 2.39 | 2.30 |
| 402 | p1K9 | 0.25 | 1.96 | 1.86 | 1.27 | 0.26 | 0.44 | 0.57 | 0.01 | 0.00 | 0.00 | 0.82 | 1.72 | 0.90 | 1.97 | 2.33 | 1.62 |
| 404 | p1K23 | 0.10 | 3.36 | 1.73 | 1.21 | 0.09 | 0.11 | 0.13 | 0.88 | 0.47 | 0.13 | 2.17 | 2.19 | 3.26 | 1.01 | 0.95 | 0.92 |
| 406 | p1K15 | 17.8 | 0.43 | 0.63 | 0.43 | 0.68 | 1.01 | 4.72 | 0.43 | 0.50 | 0.26 | 0.28 | 0.31 | 0.36 | 1.25 | 1.49 | 1.08 |
| 408 | p1K8 | 0.61 | 0.32 | 0.73 | 0.26 | 3.47 | 2.77 | 0.84 | 0.56 | 0.76 | 0.60 | 0.59 | 0.97 | 0.81 | 1.32 | 1.83 | 1.20 |
| 410 | p1M24 | 1.28 | 0.71 | 1.79 | 0.89 | 0.55 | 1.55 | 1.03 | 0.78 | 0.83 | 0.70 | 0.47 | 0.89 | 0.90 | 1.71 | 3.29 | 1.88 |
| 412 | p1K7 | 0.13 | 1.44 | 0.62 | 0.43 | 0.32 | 0.37 | 0.31 | 2.24 | 1.03 | 0.61 | 3.53 | 2.35 | 1.35 | 1.53 | 1.43 | 0.63 |
| 414 | p1K16 | 0.15 | 0.96 | 1.55 | 1.43 | 0.20 | 0.16 | 0.11 | 3.56 | 2.37 | 1.22 | 2.65 | 3.65 | 1.49 | 1.13 | 1.07 | 0.48 |
| 416 | p1K18 | 1.92 | 0.11 | 0.10 | 0.12 | 0.57 | 1.20 | 1.35 | 0.58 | 0.72 | 0.43 | 0.54 | 0.64 | 0.61 | 0.91 | 1.02 | 0.91 |
| 418 | p1N1 | 0.16 | 0.86 | 0.97 | 0.88 | 0.14 | 0.28 | 0.30 | 2.03 | 1.33 | 1.80 | 1.75 | 2.03 | 1.64 | 1.84 | 1.86 | 1.24 |
| 420 | p1K22 | 0.24 | 1.30 | 1.51 | 0.67 | 0.20 | 0.23 | 0.21 | 1.45 | 1.17 | 0.40 | 1.52 | 1.16 | 1.00 | 1.06 | 1.39 | 0.74 |
| 422 | p1K14 | 1.52 | 0.63 | 0.53 | 0.42 | 1.80 | 1.69 | 1.89 | 0.74 | 1.04 | 0.66 | 0.65 | 0.58 | 0.64 | 1.02 | 0.93 | 1.04 |
| 424 | p1K13 | 0.84 | 0.31 | 0.53 | 0.38 | 0.61 | 0.88 | 0.66 | 0.70 | 0.46 | 0.45 | 1.80 | 1.96 | 1.24 | 1.79 | 1.29 | 0.94 |
| 426 | p1J20 | 0.58 | 1.10 | 0.48 | 0.15 | 0.75 | 0.87 | 1.10 | 1.47 | 0.97 | 0.73 | 1.16 | 0.80 | 0.63 | 1.40 | 1.13 | 0.79 |
| 428 | p1J22 | 2.83 | 0.16 | 0.10 | 0.10 | 2.06 | 2.23 | 1.40 | 0.40 | 0.65 | 0.27 | 0.23 | 0.19 | 0.29 | 1.01 | 0.98 | 0.91 |
| 430 | p1K1 | 1.03 | 1.03 | 4.33 | 3.72 | 0.40 | 0.66 | 0.58 | 1.23 | 1.33 | 1.38 | 1.30 | 0.74 | 1.01 | 0.88 | 0.96 | 0.65 |
| 432 | p1K3 | 34.3 | 0.42 | 0.50 | 0.30 | 10.4 | 12.4 | 16.5 | 0.88 | 0.80 | 0.89 | 0.58 | 0.48 | 0.66 | 1.41 | 1.39 | 1.65 |
| 434 | p1J19 | 4.19 | 0.76 | 0.95 | 0.62 | 21.6 | 18.8 | 17.4 | 0.69 | 0.73 | 0.62 | 0.41 | 0.38 | 0.60 | 2.16 | 2.58 | 1.92 |
| 434 | p1K2 | 4.93 | 1.00 | 0.97 | 0.59 | 25.5 | 24.5 | 22.4 | 0.76 | 0.98 | 0.62 | 0.42 | 0.44 | 0.62 | 2.44 | 2.95 | 1.93 |
| 436 | p1K5 | 0.17 | 1.11 | 1.34 | 0.98 | 0.19 | 0.27 | 0.38 | 2.68 | 2.64 | 2.54 | 1.49 | 1.76 | 1.46 | 1.27 | 1.08 | 1.18 |
| 438 | p1J17 | 1.70 | 0.83 | 0.35 | 0.11 | 1.31 | 1.52 | 2.62 | 1.52 | 0.65 | 0.40 | 1.30 | 0.54 | 0.36 | 2.26 | 1.42 | 0.85 |
| 440 | p1J18 | 2.20 | 1.47 | 0.49 | 0.16 | 1.57 | 2.24 | 3.13 | 2.02 | 0.64 | 0.41 | 1.76 | 0.77 | 0.52 | 3.09 | 2.01 | 0.82 |
| 442 | p1J15 | 0.20 | 0.95 | 0.45 | 0.13 | 0.65 | 0.68 | 0.73 | 1.78 | 1.03 | 0.67 | 1.72 | 0.99 | 0.75 | 1.91 | 1.36 | 0.95 |
| 444 | p1K4 | 0.30 | 2.98 | 2.04 | 0.60 | 0.10 | 0.11 | 0.18 | 1.14 | 0.83 | 1.71 | 1.43 | 1.81 | 2.39 | 1.96 | 3.28 | 3.23 |
| 446 | p2A14 | 1.44 | 0.47 | 0.31 | 0.22 | 1.74 | 1.39 | 1.04 | 0.67 | 0.86 | 0.43 | 0.62 | 0.68 | 0.54 | 0.64 | 0.71 | 0.59 |
| 448 | p1J23 | 2.21 | 21.1 | 25.3 | 28.6 | 9.69 | 14.3 | 8.43 | 0.79 | 0.84 | 0.66 | 0.40 | 0.55 | 0.42 | 1.10 | 1.12 | 0.93 |
| 450 | p1J21 | 0.35 | 1.18 | 0.82 | 0.38 | 0.54 | 0.65 | 0.63 | 1.67 | 1.15 | 0.80 | 1.28 | 0.99 | 0.87 | 1.90 | 1.45 | 1.16 |
| 452 | p1J24 | 0.28 | 1.05 | 0.90 | 0.60 | 0.21 | 0.24 | 0.25 | 2.13 | 1.32 | 0.57 | 1.95 | 1.15 | 0.76 | 2.32 | 2.16 | 1.11 |
| 454 | p1J16 | 0.68 | 0.33 | 0.38 | 0.23 | 0.49 | 0.53 | 0.53 | 0.88 | 0.70 | 0.57 | 1.02 | 0.94 | 0.72 | 1.49 | 1.60 | 1.29 |
| 456 | p1J2 | 0.43 | 1.28 | 1.69 | 0.86 | 0.57 | 0.52 | 0.42 | 2.02 | 2.14 | 1.12 | 1.16 | 2.18 | 1.75 | 1.62 | 1.56 | 0.75 |
| 458 | p1J9 | 3.04 | 0.94 | 1.07 | 0.55 | 9.00 | 11.36 | 7.81 | 1.90 | 1.06 | 0.76 | 0.36 | 0.26 | 0.25 | 0.82 | 0.75 | 1.10 |
| 460 | p1J10 | 0.39 | 0.78 | 0.97 | 0.64 | 1.48 | 1.10 | 0.84 | 1.34 | 0.95 | 0.78 | 1.34 | 1.56 | 1.29 | 1.40 | 1.82 | 1.25 |
| 462 | p1J1 | 0.59 | 0.97 | 1.90 | 0.78 | 0.41 | 0.40 | 0.47 | 7.28 | 5.01 | 2.77 | 1.96 | 3.36 | 2.23 | 1.53 | 1.81 | 1.16 |
| 464 | p1J5 | 1.06 | 0.53 | 0.98 | 0.47 | 0.61 | 0.97 | 0.55 | 0.50 | 0.63 | 0.48 | 1.09 | 0.78 | 1.24 | 1.19 | 0.95 | 1.04 |
| 466 | p1J11 | 1.15 | 1.60 | 2.41 | 1.21 | 0.99 | 1.52 | 0.88 | 1.11 | 1.00 | 0.37 | 2.03 | 2.53 | 2.90 | 3.37 | 2.53 | 2.45 |
| 468 | p1J8 | 0.06 | 0.55 | 0.87 | 1.22 | 0.08 | 0.14 | 0.14 | 3.15 | 2.84 | 1.11 | 2.76 | 2.77 | 2.38 | 0.89 | 0.97 | 0.64 |
| 470 | p1I20 | 2.20 | 0.34 | 0.55 | 0.24 | 1.76 | 1.84 | 1.12 | 0.46 | 0.55 | 0.44 | 0.28 | 0.38 | 0.44 | 1.26 | 2.02 | 1.06 |
| 472 | p1J3 | 0.85 | 0.99 | 3.72 | 2.08 | 0.28 | 0.37 | 0.41 | 2.76 | 3.52 | 1.80 | 1.06 | 1.71 | 1.56 | 2.20 | 2.39 | 1.64 |
| 474 | p1J12 | 4.72 | 0.37 | 0.30 | 0.29 | 0.85 | 0.66 | 1.25 | 2.71 | 1.82 | 1.00 | 0.60 | 1.22 | 0.89 | 0.99 | 1.49 | 0.83 |
| 476 | p1I23 | 3.03 | 0.46 | 0.77 | 0.41 | 12.0 | 15.2 | 26.0 | 0.53 | 0.59 | 0.38 | 0.51 | 0.49 | 0.49 | 1.33 | 1.69 | 1.01 |
| 478 | p1J7 | 0.33 | 0.47 | 0.69 | 0.28 | 0.23 | 0.26 | 0.30 | 2.35 | 1.61 | 1.05 | 1.14 | 1.62 | 1.05 | 3.03 | 2.25 | 1.61 |
| 480 | p1I21 | 0.57 | 0.43 | 0.63 | 0.30 | 0.53 | 0.65 | 0.44 | 0.63 | 0.78 | 0.60 | 1.19 | 1.06 | 1.14 | 1.24 | 1.12 | 1.06 |
| 482 | p1I19 | 0.44 | 2.15 | 1.21 | 1.23 | 0.82 | 1.84 | 1.35 | 0.32 | 0.32 | 0.24 | 7.08 | 5.21 | 1.83 | 0.66 | 0.80 | 0.53 |
| 484 | p1J4 | 0.08 | 0.55 | 0.55 | 0.31 | 0.13 | 0.18 | 0.18 | 6.44 | 2.85 | 3.01 | 5.06 | 4.87 | 1.88 | 1.09 | 1.73 | 0.77 |
| 486 | p1I24 | 1.14 | 0.58 | 0.32 | 0.30 | 0.22 | 0.35 | 0.28 | 0.40 | 0.49 | 0.37 | 8.92 | 9.81 | 2.94 | 1.02 | 1.14 | 0.72 |
| 488 | p1I18 | 0.79 | 0.65 | 0.75 | 0.81 | 1.44 | 2.23 | 1.77 | 0.99 | 1.12 | 0.98 | 0.69 | 1.24 | 1.55 | 2.56 | 2.75 | 1.99 |

TABLE 13
Response of Novel genes to Hypoxia

| | | | HIGHEST |
|---|---|---|---|
| | | | FOLD CHANGE IN HYPOXIA |
| CLONE ID | GENE NAME | SEQ Ids | (hr hypoxia + cell type) |
| p1F6 | hypothetical protein hqp0376 protein | 337/338 | 67.4 (18 hr monocyte) |
| p1E7 | Novel metallothionein | 83/84 | 37.9 (18 hr monocyte) |
| p1D4 | hypothetical protein FLJ20500 | 25/26 | 23.8 (18 hr monocyte) |
| p1D1 | hypothetical protein FLJ10134 | 23/24 | 14.75 (18 hr neuro) |
| p1H13 | EST | 193/194 | 12.5 DOWN (18 hr mam epithelial) |
| p1F13 | hypothetical protein FLJ13356 fis, clone | 343/344 | 9.26 (18 hr monocyte) |
| | PLACE1000050 | | |
| p1H6 | EST | 191/192 | 8.42 (6 hr cardiomyocyte) |
| p1H17 | EST | 171/172 | 8.33 DOWN (18 hr mam epithelial) |
| p1E14 | unknown mRNA (schizophrenia-linked) | 97/98 | 7.79 (6 hr mam epithelial) |
| p1P14 | hypothetical protein KIAA1745 | 91/92 | 7.32 (18 hr renal epithelial) |
| p1H19 | EST | 195/196 | 7.14 DOWN (18 hr cardiomyocyte) |
| p1D11 | EST | 135/136 | 6.90 (6 hr mam epithelial) |
| p1D17 | hypothetical protein KIAA1745 | 91/92 | 6.74 (6 hr cardiomyocyte) |
| p1F9 | hypothetical protein KIAA0742 | 19/20 | 6.64 (18 hr monocyte) |
| p1D2 | hypothetical protein FLJ10134 | 23/24 | 6.62 (18 hr neuro) |
| p1H21 | hypothetical protein FLJ13511 | 163/164 | 6.61 (18 hr monocyte) |
| p1D16 | cDNA FLJ20308 fis, clone HEP07264 | 33/34 | 6.29 (18 hr neuroblastoma) |
| p1D12 | hypothetical protein KIAA1376 | 29/30 | 5.98 (6 hr cardiomyocyte) |
| p1H3 | EST | 215/216 | 5.88 DOWN (18 hr mam epithelial) |
| p1H10 | EST | 189/190 | 4.98 (6 hr cardiomyocyte) |
| p1D18 | cDNA FLJ13443 fis, clone PLACE1002853 | 127/128 | 4.84 (6 hr cardiomyocyte) |
| p1H4 | EST | 213/214 | 4.76 DOWN (18 hr mam epithelial) |
| p1I4 | hypothetical protein HSPC196 | 53/54 | 4.54 DOWN (18 he mam epithelial) |
| p1G20 | cDNA YO23H03 | 203/204 | 4.17 DOWN (18 hr cardiomyocyte) |
| p1I15 | hypothetical protein CGI-117 | 47/48 | 4.17 DOWN (18 hr adipocyte) |
| p1F8 | hypothetical protein KIAA0914 | Oct. 9 | 3.88 (6 hr mam epithelial) |
| p1H20 | EST | 179/180 | 3.84 DOWN (18 hr cardiomyocyte) |
| p1I22 | hypothetical protein KIAA1429 | 37/38 | 3.70 DOWN (18 hr mam epithelial) |
| p1E16 | cDNA DKFZp586E1624 | 65/66 | 3.56 (6 hr endothelial) |
| p1H15 | EST | 177/178 | 3.45 DOWN (18 hr mam epithelial) |
| p1F5 | hypothetical protein FLJ20281 | Dec. 11 | 3.36 (6 hr mam epithelial) |
| p1E1 | EST | 123/124 | 3.24 (6 hr cardiomyocyte) |
| p1H7 | EST | 175/176 | 3.13 DOWN (18 hr mam epithelial) |
| p1D19 | EST | 143/144 | 2.98 (6 hr cardiomyocyte) |
| p1F21 | cDNA FLJ14342 fis, clone THYRO1000569 | 17/18 | 2.92 (18 hr monocyte) |
| p1H12 | EST | 173/174 | 2.84 (6 hr hepatocyte) |
| p1F2 | hypothetical protein FLJ20037 | Apr. 3 | 2.84 (18 hr monocyte) |
| p1H23 | cDNA FLJ21094 fis, clone CAS03807 | 187/188 | 2.78 DOWN (18 hr neuroblastoma) |
| p1I13 | hypothetical protein FLJ11100 | 43/44 | 2.78 DOWN (18 hr adipocyte) |
| p1D20 | hypothetical protein KIAA1125 | 139/140 | 2.73 (6 hr renal epithelial) |
| p1E11 | EST | 109/110 | 2.73 (6 hr hepatocyte) |
| p1D9 | hypothetical protein DKFZP564D116 | 27/28 | 2.71 (6 hr adipocyte) |
| p1H5 | hypothetical protein FLJ22690 | 205/206 | 2.55 (6 hr cardiomyocyte) |
| p1D24 | EST | 117/118 | 2.55 (18 hr renal epithelial) |
| p1E12 | hypothetical protein DKFZP434E1723 | 69/70 | 2.49 (6 hr mam epithelial) |
| p1F10 | hypothetical protein DKFZp434P0116 | Jun. 5 | 2.45 (6 hr mam epithelial) |
| p1G22 | EST | 197/198 | 2.38 DOWN (18 hr mam epithelial) |
| p1E4 | EST | 125/126 | 2.31 (18 hr monocyte) |
| p1I3 | hypothetical protein FLJ11656 | 153/154 | 2.27 DOWN (18 hr adipocyte) |
| p1F12 | EST | Feb. 1 | 2.27 (6 hr hepatocyte) |
| p1G18 | Mitochondrion sequence | 211/212 | 2.17 (18 hr neuroblastoma) |
| p1E23 | cDNA FLJ14041 fis, clone HEMBA1005780 | 111/112 | 2.16 (18 hr monocyte) |
| p1E9 | novel PI-3-kinase adapter | 79/80 | 2.15 (18 hr monocyte) |
| p1G7 | EST | 281/282 | 2.14 (18 hr monocyte) |
| p1J13 | hypothetical nuclear factor SBBI22 | 35/36 | 2.13 DOWN (18 hr adipocyte) |
| p1E19 | EST | 105/106 | 2.13 (6 hr hepatocyte) |
| p1E15 | cDNA YI27F12 | 107/108 | 2.12 (18 hr monocyte) |
| p1F23 | hypothetical protein LOC51014 | 289/290 | 2.10 (6 hr endothelial) |
| p2A14 | EST | 445/446 | 2.08 DOWN (18 hr mam epithelial) |
| p1I17 | hypothetical protein FLJ20644 | 45/46 | 2.08 (6 hr endothelial) |
| p1E8 | cDNA: FLJ22249 fis, clone HRC02674 | 61/62 | 2.07 (6 hr endothelial) |
| p1H1 | hypothetical protein FLJ10826 | 201/202 | 2.07 (18 hr endothelial) |
| p1I10 | EST | 155/156 | 2.04 DOWN (18 hr mam epithelial) |
| p1I5 | hypothetical protein FLJ10815 | 41/42 | 2.04 (6 hr cardiomyocyte) |
| p1E20 | hypothetical protein FLJ20421 | 99/100 | 2.01 (6 hr renal epithelial) |
| p1C23 | cDNA FLJ12832 fis, clone NT2RP2003137 | 133/134 | 2.01 (6 hr monocyte) |
| p1J16 | cDNA: FLJ23019 fis, clone LNG00916 | 453/454 | 2.00 (6 hr cardiomyocyte) |
| p1I12 | hypothetical protein MGC4549 | 151/152 | 1.96 DOWN (18 hr mam epithelial) |
| p1F1 | EST | 81/82 | 1.96 (18 hr monocyte) |
| p1E22 | cDNA FLJ13618 fis, clone PLACE1010925 | 161/162 | 1.95 (6 hr monocyte) |
| p1D21 | hypothetical protein FLJ22622 | 129/130 | 1.95 (6 hr cardiomyocyte) |
| p1F19 | hypothetical protein KIAA0212 | Aug. 7 | 1.95 (18 hr monocyte) |
| p1F18 | hypothetical protein KIAA0876 | 13/14 | 1.92 DOWN (6 hr adipocyte) |
| p1H9 | EST | 185/186 | 1.91 (18 hr monocyte) |
| p1F11 | hypothetical protein LOC51754 | 323/324 | 1.90 (6 hr macrophage) |
| p1I2 | cDNA FLJ11302 fis, clone PLACE1009971 | 149/150 | 1.88 (18 hr monocyte) |
| p1G21 | EST | 199/200 | 1.85 DOWN (6 hr macrophage) |
| p2A24 | EST | 101/102 | 1.85 DOWN (18 hr adipocyte) |
| p1E13 | hypothetical protein PRO0823 | 21/22 | 1.85 DOWN (18 hr adipocyte) |
| p1E10 | cDNA FLJ11041 fis, clone PLACE1004405 | 71/72 | 1.84 (6 hr endothelial) |
| p1I14 | cDNA DKFZp564D016 | 147/148 | 1.80 (6 hr monocyte) |
| p1J6 | hypothetical protein FLJ10206 | 39/40 | 1.78 (6 hr mam epihelial) |
| p1F3 | hypothetical protein LOC94951 | 333/334 | 1.75 (6 hr renal epithelial) |
| p1I16 | hypothetical protein KIAA1668 | 57/58 | 1.69 (6 hr mam epithelial) |
| p1H16 | EST | 183/184 | 1.64 (18 hr renal epithelial) |
| p1E17 | cDNA FLJ31668 fis, clone NT2RI2004916 | 103/104 | 1.63 (6 hr cardiomyocyte) |
| p1I8 | hypothetical protein FLJ11296 | 55/56 | 1.61 (18 hr macrophage) |
| p1H14 | EST | 167/168 | 1.45 (6 hr renal epithelial) |

TABLE 14
Response of Novel genes to Hypoxia

| | | | HIGHEST |
|---|---|---|---|
| | | | FOLD CHANGE IN HYPOXIA |
| CLONE ID | GENE NAME | SEQ Ids | (hr hypoxia + cell type) |
| p1D6 | ERO1 (S. cerevisiae)-like | 67/68 | 11.30 (18 hr fibroblast) |
| p1D10 | Insulin induced protein 2 | 75/76 | 8.14 (18 hr renal epithelial) |
| p1H2 | Fatty acid binding protein 5 | 209/210 | 7.14 DOWN (18 hr neuroblastoma) |
| p1H18 | Ubiquitin specific protease 7 | 157/158 | 7.14 DOWN (18 hr mam epithelial) |
| p1D22 | MAX-interacting protein 1 | 119/120 | 6.68 (18 hr renal epithelial) |
| p1C24 | SLC25A19 | 93/94 | 6.13 (18 hr macrophage) |
| p1E3 | CYP1B1 | 137/138 | 5.88 DOWN (18 hr renal epithelial) |
| p1G19 | Mitochondrion sequence | 207/208 | 5.88 DOWN (18 hr mam epithelial) |
| p1D14 | C1orf12 | 89/90 | 5.68 (6 hr cardiomyocyte) |
| p1H8 | ABL | 181/182 | 4.76 DOWN (18 hr cardiomyocyte) |
| p1E6 | EGL nine (C. elegans) homolog 3 | 85/86 | 4.63 (18 hr mam epithelial) |
| p1D13 | Adenylate kinase 3 | 77/78 | 4.58 (6 hr cardiomyocyte) |
| p1H24 | Nucleolar phosphoprotein Nopp34 | 159/160 | 4.40 (6 hr cardiomyocyte) |
| p1D15 | TRIP-Br2 | 31/32 | 4.09 (18 hr renal epithelial) |
| p1F7 | Spectrin, beta, non-erythrocytic 1 | 15/16 | 2.65 (6 hr endothelial) |
| p1E5 | Hepcidin antimicrobial peptide | 141/142 | 2.59 (6 hr macrophage) |
| p1C22 | CD84-H1 | 131/132 | 2.58 (6 hr cardiomyocyte) |
| p1E2 | Mannosidase, alpha, class 1A, member 1 | 121/122 | 2.56 DOWN (18 hr neuroblastoma) |
| p1C21 | Tubulin, beta, 4 | 73/74 | 2.51 (6 hr cardiomyocyte) |
| p1D3 | Serine carboxypeptidase 1 | 95/96 | 2.49 (18 hr fibroblast) |
| p1H11 | Carboxypeptidase M | 169/170 | 2.18 (18 hr monocyte) |
| p1E18 | Plexin C1 | 63/64 | 2.15 (6 hr hepatocyte) |
| p2B1 | PRAME | 87/88 | 2.13 DOWN (18 hr fibroblast) |
| p1E21 | Glutamate-cysteine ligase, modifier subunit | 113/114 | 2.04 (6 hr hepatocyte) |
| p1I11 | SECIS binding protein 2 | 59/60 | 2.00 DOWN (18 hr endothelial) |
| p1I1 | Ribosomal RNA intergenic spacer | 165/166 | 1.92 DOWN (18 hr neuroblastoma) |
| p1I7 | Uridine 5′ monophosphate hydrolase 1 | 49/50 | 1.77 (18 hr monocyte) |
| p1D23 | PTEN | 115/116 | 1.74 (6 hr renal epithelial) |
| p1D5 | ERO1 (S. cerevisiae)-like | 67/68 | 1.72 (6 hr mam epithelial) |
| p2A15 | Sialyltransferase | 145/146 | 1.61 DOWN (6 hr monocyte) |

TABLE 15
Genes with increased expression by macrophage activation

| | | | mRNA EXPRESSION | | | | | |
|---|---|---|---|---|---|---|---|---|
| | SEQ ID | | (experimental condition) | | | | | |
| Clone | NO: | Gene Name | #1 | #2 | #3 | #4 | #5 | #6 |
| p1K8 | 407/408 | SCYA4 | 0.82 | 0.40 | 1.15 | 0.38 | 91.4 | 68.4 |
| p1B16 | 251/252 | Interleukin 8 | 0.75 | 1.13 | 0.47 | 0.41 | 42.8 | 28.1 |
| p1B15 | 251/252 | Interleukin 8 | 0.85 | 1.12 | 0.44 | 0.37 | 47.4 | 22.5 |
| p1I21 | 479/480 | SCYA8 | 0.54 | 0.18 | 1.15 | 0.32 | 19.6 | 12.2 |
| p1I20 | 469/470 | SCYA3L | 0.92 | 0.41 | 1.00 | 0.30 | 29.4 | 22.8 |
| p1N17 | 237/238 | COX-2 | 0.90 | 1.00 | 0.84 | 0.84 | 18.9 | 20.3 |
| p1J16 | 453/454 | cDNA: FLJ23019 fis, | 0.92 | 0.66 | 0.91 | 1.15 | 14.4 | 14.9 |
| | | clone LNG00916 | | | | | | |
| p1I7 | 49/50 | Uridine 5′ | 1.13 | 0.57 | 0.99 | 0.52 | 17.6 | 23.7 |
| | | monophosphate | | | | | | |
| | | hydrolase 1 | | | | | | |
| p1B14 | 251/252 | Interleukin 8 | 0.71 | 1.20 | 0.51 | 0.47 | 10.1 | 21.4 |
| p1E10 | 71/72 | cDNA FLJ11041 fis, | 0.66 | 0.74 | 1.15 | 0.81 | 8.30 | 12.1 |
| | | clone PLACE1004405 | | | | | | |
| p2L23 | 397/398 | endothelin 1 | 1.02 | 0.62 | 0.74 | 0.50 | 11.4 | 10.1 |
| p1D19 | 143/144 | EST | 0.63 | 0.52 | 1.00 | 1.16 | 5.46 | 4.73 |
| p1K3 | 431/432 | Pleckstrin | 1.14 | 0.70 | 0.73 | 0.54 | 6.49 | 2.34 |
| p1C9 | 373/374 | RAB-8b protein | 0.95 | 0.81 | 0.77 | 0.94 | 5.11 | 4.53 |
| p1I24 | 485/486 | GRO1 | 0.90 | 0.72 | 0.78 | 1.04 | 4.69 | 2.96 |
| p1G3 | 317/318 | B-cell translocation | 0.70 | 1.00 | 0.57 | 1.14 | 3.51 | 3.79 |
| | | gene 1 | | | | | | |
| p1B1 | 243/244 | Metallothionein 1G | 0.51 | 1.00 | 0.66 | 1.85 | 2.50 | 3.83 |
| p1J11 | 465/466 | Fatty-acid-Coenzyme A | 0.69 | 0.51 | 1.36 | 0.91 | 3.07 | 2.97 |
| | | ligase, long-chain 2 | | | | | | |
| p1F17 | 329/330 | P8 protein (candidate of | 0.26 | 1.78 | 0.16 | 0.88 | 1.16 | 2.59 |
| | | metastasis 1) | | | | | | |
| p1F4 | 339/340 | CYP1 | 0.60 | 1.04 | 0.77 | 1.15 | 2.52 | 4.22 |
| p1D10 | 75/76 | Insulin induced protein | 0.49 | 1.00 | 0.48 | 1.23 | 1.63 | 4.96 |
| | | 2 | | | | | | |
| p1E7 | 83/84 | Novel metallothionein | 0.49 | 1.26 | 0.70 | 1.11 | 1.32 | 2.89 |
| p1D24 | 117/118 | EST | 0.58 | 0.71 | 1.00 | 1.32 | 1.56 | 2.59 |
| p1I19 | 481/482 | GR02 | 0.99 | 1.00 | 0.69 | 0.55 | 2.65 | 2.29 |
| p1E22 | 161/162 | cDNA FLJ13618 fis, | 1.19 | 0.77 | 0.85 | 0.51 | 3.09 | 2.41 |
| | | clone PLACE1010925 | | | | | | |
| p1F6 | 337/338 | hypothetical protein | 0.44 | 1.08 | 0.47 | 1.06 | 1.11 | 2.47 |
| | | hqp0376 | | | | | | |
| p1J7 | 477/478 | Sjogren syndrome | 1.06 | 0.74 | 0.85 | 0.63 | 2.65 | 2.95 |
| | | antigen B | | | | | | |
| p1B19 | 235/236 | plasminogen activator | 0.59 | 1.29 | 0.45 | 1.17 | 1.46 | 3.46 |
| | | inhibitor, type 1 | | | | | | |
| p1F24 | 297/298 | Glia-derived nexin | 0.65 | 0.68 | 0.99 | 1.10 | 1.62 | 1.80 |
| p1P5 | 395/396 | SCYA2 | 0.94 | 0.13 | 3.81 | 0.42 | 2.27 | 1.00 |
| p1A22 | 263/264 | Adenylate kinase 3 | 0.57 | 1.30 | 0.58 | 1.64 | 1.34 | 2.74 |
| p1A23 | 265/266 | Metallothionein 2A | 0.55 | 0.89 | 0.95 | 1.01 | 1.23 | 4.38 |
| p1A24 | 239/240 | Metallothionein 1H | 0.50 | 0.84 | 1.03 | 1.02 | 1.08 | 1.60 |
| p1P3 | 221/222 | PDGFB | 0.47 | 2.06 | 0.31 | 1.99 | 1.00 | 1.49 |
| p1A7 | 345/346 | SLC31A2 | 1.10 | 0.92 | 0.84 | 0.93 | 2.30 | 2.32 |
| p1P14 | 91/92/92a | Semaphorin 4b | 0.47 | 2.52 | 0.95 | 4.04 | 0.96 | 3.88 |

Legend
mRNA expression values in the 6 experimental conditions (#1 no cytokines/normoxia, #2 no cytokines/hypoxia, #3 IL-10/normoxia, #4 IL-10/hypoxia, #5 LPS/IFN/normoxia, #6 LPS/IFN/hypoxia) are shown as values referenced to the median value of that gene throughout all 6 experimental conditions.

TABLE 16
Genes down-regulated by macrophage activation

| | | | mRNA EXPRESSION | | | | | |
|---|---|---|---|---|---|---|---|---|
| | SEO ID | | (experimental condition) | | | | | |
| Clone | NO: | Gene Name | #1 | #2 | #3 | #4 | #5 | #6 |
| p1H13 | 193/194 | EST | 1.35 | 1.41 | 1.00 | 0.91 | 0.44 | 0.68 |
| p1E4 | 125/126 | EST | 1.22 | 1.13 | 1.09 | 0.96 | 0.40 | 0.40 |
| p1G7 | 281/282 | EST | 1.30 | 1.44 | 1.01 | 1.51 | 0.54 | 0.82 |
| p1E1 | 123/124 | EST | 1.21 | 1.64 | 0.94 | 1.35 | 0.51 | 0.63 |
| p1D18 | 127/128 | cDNA FLJ13443 fis, | 1.61 | 2.60 | 0.57 | 1.33 | 0.26 | 0.24 |
| | | clone PLACE1002853 | | | | | | |
| p1I2 | 149/150 | cDNA FLJ11302 fis, | 2.39 | 1.23 | 1.07 | 0.54 | 0.45 | 0.43 |
| | | clone PLACE1009971 | | | | | | |
| p1G20 | 203/204 | cDNA YO23H03 | 1.45 | 0.73 | 1.60 | 1.12 | 0.57 | 0.44 |
| p1D21 | 129/130 | hypothetical protein | 1.41 | 1.72 | 0.82 | 1.26 | 0.14 | 0.14 |
| | | FLJ22622 | | | | | | |
| p1F8 | 9/10 | hypothetical protein | 1.34 | 4.14 | 0.77 | 2.74 | 0.13 | 0.25 |
| | | KIAA0914 | | | | | | |
| p1D16 | 33/34 | hypothetical protein | 1.31 | 2.36 | 1.00 | 1.59 | 0.29 | 0.67 |
| | | FLJ20308 | | | | | | |
| p1F3 | 333/334 | hypothetical protein | 1.63 | 2.21 | 0.92 | 1.00 | 0.42 | 0.42 |
| | | XP_017131 | | | | | | |
| p1D12 | 29/30 | hypothetical protein | 0.89 | 2.62 | 0.79 | 2.07 | 0.28 | 2.61 |
| | | KIAA1376 | | | | | | |
| p1I4 | 53/54 | hypothetical protein | 1.95 | 1.06 | 1.20 | 0.57 | 0.63 | 0.28 |
| | | HSPC196 | | | | | | |
| p1D9 | 27/28 | hypothetical protein | 1.63 | 0.94 | 1.25 | 0.96 | 0.55 | 0.85 |
| | | DKFZP564D116 | | | | | | |
| p1F9 | 19/20 | hypothetical protein | 0.94 | 3.54 | 0.60 | 1.74 | 0.33 | 1.74 |
| | | KIAA0742 | | | | | | |
| p1F11 | 323/324 | hypothetical protein | 1.67 | 1.91 | 1.00 | 0.86 | 0.60 | 0.59 |
| | | LOC51754 | | | | | | |
| p1I15 | 47/48 | hypothetical protein CGI- | 1.31 | 0.62 | 1.86 | 1.26 | 0.49 | 0.76 |
| | | 117 | | | | | | |
| p1E13 | 21/22 | hypothetical protein | 1.15 | 0.93 | 1.15 | 1.08 | 0.44 | 0.24 |
| | | PRO0823 | | | | | | |
| p1F10 | “5/6” | hypothetical protein | 2.16 | 1.05 | 1.54 | 0.83 | 0.83 | 0.67 |
| | | DKFZp434P0116 | | | | | | |
| p1D1 | 23/24 | hypothetical protein | 0.86 | 1.70 | 0.61 | 2.42 | 0.35 | 1.61 |
| | | FLJ10134 | | | | | | |
| p1I5 | 41/42 | hypothetical protein | 1.49 | 1.00 | 1.30 | 0.83 | 0.43 | 0.37 |
| | | FLJ10815 | | | | | | |
| p1G13 | 293/294 | ABCA1 | 1 | 1.05 | 1.22 | 1.07 | 0.43 | 0.46 |
| p1B9 | 313/314 | adipophilin | 1.01 | 3.74 | 1.32 | 2.08 | 0.02 | 0.2 |
| p1B7 | 313/314 | adipophilin | 1.57 | 3.44 | 0.77 | 1.17 | 0.21 | 0.45 |
| p1B6 | 313/314 | adipophilin | 1.24 | 2.45 | 0.8 | 1.51 | 0.38 | 0.52 |
| p1B8 | 313/314 | adipophilin | 1.11 | 1.87 | 1.01 | 1.04 | 0.55 | 0.59 |
| p1K7 | 411/412 | ATP-binding cassette E1 | 1.34 | 0.74 | 1.58 | 1.05 | 0.62 | 0.48 |
| p1J23 | 447/448 | Calgranulin A | 1.21 | 0.94 | 3.35 | 2.8 | 0.58 | 0.86 |
| p1K18 | 415/416 | Colony-stimulating | 2.01 | 0.84 | 1.7 | 0.88 | 1 | 0.74 |
| | | factor 1 | | | | | | |
| p1C2 | 331/332 | CXCR4 | 1.01 | 3.76 | 0.46 | 1.47 | 0.08 | 1.13 |
| p1C1 | 331/332 | CXCR4 | 1.05 | 3.64 | 0.39 | 1.63 | 0.27 | 0.98 |
| p1G12 | 321/322 | Cyclin G2 | 0.85 | 2.17 | 0.6 | 1.29 | 0.28 | 1.33 |
| p1F16 | 325/326 | CYP1B1 | 1.37 | 0.96 | 1.67 | 0.66 | 0.64 | 0.48 |
| p1C7 | 369/370 | D123 | 1.69 | 1.33 | 1.1 | 0.7 | 0.65 | 0.83 |
| p1G17 | 315/316 | Early development | 0.97 | 2.47 | 1.12 | 2.24 | 0.29 | 0.85 |
| | | regulator 2 | | | | | | |
| p1I23 | 475/476 | Ecotropic viral | 1.39 | 1.25 | 1.11 | 1.72 | 0.18 | 0.22 |
| | | integration site 2A | | | | | | |
| p1A14 | 257/258 | Enolase 1 | 0.99 | 3.22 | 1.19 | 2.46 | 0.13 | 0.37 |
| p1A10 | 273/274 | Enolase 2 | 1.17 | 5.28 | 0.59 | 3.77 | 0.49 | 1.08 |
| p1D6 | 67/68 | ERO1 (S. cerevisiae)-like | 0.84 | 3.02 | 0.97 | 2.87 | 0.32 | 1.58 |
| p1A11 | 253/254 | GAPDH | 1.21 | 2.41 | 0.93 | 1.31 | 0.32 | 0.81 |
| p1A12 | 253/254 | GAPDH | 1.09 | 1.97 | 1 | 1.49 | 0.41 | 0.96 |
| p1K22 | 419/420 | GPR44 | 1.24 | 1.03 | 1.42 | 0.93 | 0.56 | 0.48 |
| p1C18 | 269/270 | Granulin | 1.28 | 1.59 | 0.96 | 1.03 | 0.56 | 0.72 |
| p1C17 | 269/270 | Granulin | 1.58 | 1.6 | 0.62 | 0.41 | 0.76 | 0.94 |
| p1A15 | 249/250 | Hexokinase-2 | 0.89 | 3.88 | 0.68 | 3.11 | 0.38 | 2.02 |
| p1C13 | 381/382 | Jk-recombination signal | 1.11 | 1.18 | 1.43 | 1.98 | 0.32 | 0.73 |
| | | binding protein | | | | | | |
| p1A8 | 223/224 | Lactate dehydrogenase A | 0.7 | 2.25 | 1.4 | 1.44 | 0.26 | 1.32 |
| p1A9 | 223/224 | Lactate dehydrogenase A | 0.77 | 1.85 | 1.15 | 1.68 | 0.32 | 1.19 |
| p1G5 | 279/280 | MAX-interacting protein | 1.24 | 5.5 | 0.9 | 4.48 | 0.34 | 0.97 |
| | | 1 | | | | | | |
| p1D22 | 119/120 | MAX-interacting protein | 1.2 | 3.86 | 0.52 | 3.44 | 0.37 | 0.91 |
| | | 1 | | | | | | |
| p1G18 | 211/212 | Mitochondrion sequence | 1.27 | 1.12 | 1 | 1.31 | 0.57 | 0.77 |
| p1K23 | 403/404 | MYC | 1.37 | 0.77 | 2.39 | 1.09 | 0.54 | 0.35 |
| p1E20 | 99/100 | Myo-inositol | 1.12 | 1.28 | 1.02 | 0.99 | 0.48 | 0.61 |
| | | monophosphatase A3 | | | | | | |
| p1B20 | 267/268 | Osteopontin | 1.13 | 1.58 | 0.99 | 1.52 | 0.1 | 0.4 |
| p1F13 | 343/344 | Papillomavirus regulatory | 0.98 | 5.02 | 0.44 | 6.79 | 0.09 | 2.43 |
| | | factor PRF-1 | | | | | | |
| p1A13 | 255/256 | Phosphoglycerate kinase | 1.04 | 2.45 | 1.23 | 1.83 | 0.2 | 0.9 |
| | | 1 | | | | | | |
| p1G9 | 305/306 | PI-3-kinase, catalytic, | 1.46 | 1.88 | 0.75 | 1.17 | 0.44 | 0.47 |
| | | beta polypeptide | | | | | | |
| p1E18 | 63/64 | Plexin C1 | 1.72 | 1.79 | 1 | 0.85 | 0.69 | 0.35 |
| p1C11 | 377/378 | polyubiquitin | 1.13 | 1.79 | 0.79 | 1.14 | 0.5 | 0.84 |
| p1B3 | 231/232 | Proline 4-hydroxylase, | 0.94 | 1.38 | 1.03 | 1.58 | 0.43 | 0.89 |
| | | alpha polypeptide I | | | | | | |
| p1B4 | 349/350 | Proline 4-hydroxylase, | 0.9 | 1.46 | 1.05 | 1.41 | 0.44 | 1 |
| | | alpha polypeptide II | | | | | | |
| p1B22 | 355/356 | Protease, serine, 11 | 1.3 | 1.1 | 1.26 | 0.92 | 0.64 | 0.7 |
| p1C10 | 375/376 | Regulator of G-protein | 1.42 | 1.68 | 0.94 | 1.55 | 0.47 | 0.95 |
| | | signalling 1 | | | | | | |
| p1D3 | 95/96 | Serine carboxypeptidase | 1.22 | 1.07 | 1.07 | 1.09 | 0.33 | 0.88 |
| | | 1 | | | | | | |
| p1F15 | 341/342 | SHB adaptor protein | 1.04 | 1.61 | 0.94 | 1.72 | 0.43 | 0.54 |
| p1A5 | 311/312 | SLC2A5 | 0.71 | 2.6 | 1.06 | 2.09 | 0.34 | 1.09 |
| p1G4 | 307/308 | SLC5A3 | 1.12 | 1.44 | 0.93 | 1.31 | 0.33 | 0.62 |
| p1A20 | 261/262 | Triosephosphate | 0.97 | 2.06 | 1.09 | 2.24 | 0.17 | 0.66 |
| | | isomerase 1 | | | | | | |
| p1D15 | 31/32 | TRIP-Br2 | 1.16 | 1.4 | 1.1 | 1.25 | 0.47 | 0.46 |
| p1K4 | 443/444 | TSC-22 | 1.44 | 1 | 1.55 | 0.7 | 0.6 | 0.57 |

TABLE 17
Genes responsive to IL-10 (increased or decreased)
but not affected significantly by LPS + IFN

| | | | mRNA EXPRESSION | | | | | |
|---|---|---|---|---|---|---|---|---|
| | SEQ ID | | (experimental condition) | | | | | |
| Clone | NO: | Gene Name | #1 | #2 | #3 | #4 | #5 | #6 |
| p1H8 | 181/182 | ABL | 1.02 | 0.96 | 6.65 | 5.25 | 0.86 | 0.73 |
| p1E15 | 107/108 | cDNA YI27F12 | 0.48 | 0.77 | 1.69 | 2.45 | 0.78 | 1.44 |
| p2A14 | 445/446 | EST | 1.06 | 0.74 | 2.78 | 3.09 | 1.06 | 0.82 |
| p1H6 | 191/192 | EST | 1.01 | 0.84 | 2.47 | 2.05 | 0.93 | 0.83 |
| p1E5 | 141/142 | Hepcidin antimicrobial peptide | 0.84 | 0.73 | 1.91 | 1.68 | 0.58 | 2.16 |
| p1I12 | 151/152 | hypothetical protein MGC4549 | 1.07 | 0.67 | 2.34 | 2.53 | 1.11 | 0.74 |
| p1D8 | 271/272 | Hypoxia-inducible protein 2 | 0.65 | 1.00 | 1.51 | 1.89 | 0.71 | 2.05 |
| p1K14 | 421/422 | Keratin 6B | 1.03 | 0.68 | 3.80 | 3.28 | 0.97 | 0.76 |
| p1J22 | 427/428 | Neutral sphingomyelinase (N-SMase) | 0.94 | 0.79 | 5.59 | 3.52 | 0.91 | 1.29 |
| | | activation associated factor | | | | | | |
| p1G15 | 287/288 | Phosphoglucomutase 1 | 0.82 | 1.20 | 1.83 | 1.90 | 0.61 | 1.05 |
| p1A2 | 247/248 | SLC2A3 | 0.37 | 3.31 | 1.00 | 3.32 | 0.49 | 2.63 |
| p1A3 | 247/248 | SLC2A3 | 0.39 | 2.45 | 1.00 | 2.65 | 0.20 | 1.50 |
| p1K2 | 433/434 | CFFM4 | 1.30 | 0.98 | 0.51 | 0.59 | 1.11 | 0.91 |
| p1C4 | 363/364 | FGF receptor activating protein 1 | 1.02 | 0.96 | 0.50 | 0.63 | 1.16 | 1.31 |

TABLE 18
Genes up-regulated in human tumors. Individual patients are denoted by the letters E,F,G,H and K.

| | | | Ovary | Ovary | Ovary | Ovary | Ovary | Ovary | Breast | Breast | Breast | Breast |
|---|---|---|---|---|---|---|---|---|---|---|---|---|
| | | | nor | tum | nor | tum | nor | tum | nor | tum | nor | tum |
| Clone | Gene Name | Seq ID | E | E | F | F | G | G | H | H | K | K |
| p1H8 | ABL | 182 | 0.73 | 2.21 | 0.72 | 1.48 | 1.15 | 3.15 | 2.41 | 2.01 | 2.61 | 1.00 |
| p1B6 | adipophilin | 314 | 0.44 | 1.57 | 0.51 | 0.37 | 1.30 | 0.99 | 0.58 | 0.78 | 0.82 | 1.06 |
| p1A19 | Aldolase C | 260 | 0.27 | 1.00 | 0.74 | 1.06 | 0.40 | 1.49 | 0.62 | 0.47 | 0.49 | 2.18 |
| p1C2 | CXCR4 | 332 | 0.29 | 0.91 | 1.03 | 1.41 | 2.43 | 2.80 | 2.71 | 0.95 | 1.81 | 0.59 |
| p1K1 | Cyclophilin F | 430 | 0.60 | 0.71 | 1.11 | 0.80 | 0.61 | 1.85 | 0.76 | 0.76 | 0.95 | 1.66 |
| p1E3 | CYP1B1 | 138 | 0.32 | 0.06 | 0.45 | 1.47 | 1.05 | 0.16 | 1.00 | 0.38 | 1.20 | 0.16 |
| p1F16 | CYP1B1 | 326 | 0.60 | 0.17 | 0.67 | 2.30 | 1.65 | 0.24 | 1.00 | 0.55 | 1.82 | 0.24 |
| p1C8 | Dec1 | 372 | 3.93 | 0.66 | 1.85 | 1.10 | 1.37 | 0.56 | 0.94 | 0.53 | 0.87 | 5.55 |
| p1A14 | Enolase 1 | 258 | 0.10 | 0.46 | 1.00 | 1.26 | 0.41 | 1.47 | 0.36 | 0.81 | 0.53 | 0.61 |
| p1A10 | Enolase 2 | 274 | 0.63 | 0.64 | 1.48 | 3.23 | 0.80 | 2.67 | 0.96 | 0.92 | 0.92 | 0.52 |
| p1D6 | ERO1 (S. cerevisiae)- | 68 | 0.30 | 1.61 | 0.73 | 1.32 | 0.77 | 0.31 | 0.66 | 0.42 | 1.10 | 1.02 |
| | like | | | | | | | | | | | |
| p1E19 | EST | 106 | 1.00 | 2.04 | 1.38 | 1.30 | 0.20 | 1.19 | 2.38 | 2.49 | 1.19 | 1.78 |
| p1H15 | EST | 178 | 0.22 | 0.46 | 0.71 | 1.07 | 0.82 | 2.57 | 2.62 | 1.22 | 3.66 | 1.07 |
| p1H16 | EST | 184 | 1.57 | 2.66 | 1.44 | 1.42 | 0.74 | 2.65 | 2.32 | 1.91 | 1.70 | 1.95 |
| p1H17 | EST | 172 | 0.65 | 2.20 | 1.00 | 1.62 | 0.95 | 2.64 | 2.95 | 2.32 | 2.98 | 0.93 |
| p1H20 | EST | 180 | 0.81 | 2.39 | 0.98 | 1.61 | 0.78 | 2.91 | 2.83 | 1.62 | 2.64 | 1.17 |
| p1H3 | EST | 216 | 0.10 | 0.38 | 0.78 | 1.14 | 0.88 | 2.49 | 2.26 | 1.18 | 3.08 | 0.89 |
| p1C4 | FGF receptor | 364 | 0.53 | 0.77 | 0.91 | 1.00 | 1.04 | 0.98 | 0.81 | 1.27 | 0.72 | 2.43 |
| | activating protein 1 | | | | | | | | | | | |
| p1A11 | GAPDH | 254 | 0.04 | 0.74 | 1.50 | 2.84 | 0.86 | 1.57 | 0.27 | 1.12 | 0.51 | 0.69 |
| p1C6 | Glucose phosphate | 368 | 0.21 | 0.86 | 1.59 | 2.26 | 0.65 | 1.94 | 0.47 | 0.79 | 0.61 | 1.00 |
| | isomerase | | | | | | | | | | | |
| p1E21 | Glutamate-cysteine | 114 | 1.26 | 2.38 | 1.35 | 1.23 | 0.27 | 1.51 | 2.05 | 4.12 | 1.00 | 1.78 |
| | ligase, modifier | | | | | | | | | | | |
| | subunit | | | | | | | | | | | |
| p1C18 | Granulin | 270 | 0.44 | 0.91 | 1.00 | 0.83 | 0.71 | 0.88 | 0.60 | 0.61 | 0.73 | 2.72 |
| p1D21 | hypothetical protein | 130 | 0.39 | 8.14 | 0.53 | 0.59 | 0.92 | 0.54 | 0.87 | 1.71 | 0.99 | 1.00 |
| | FLJ22622 | | | | | | | | | | | |
| p1F11 | hypothetical protein | 324 | 0.53 | 0.77 | 1.01 | 0.97 | 0.61 | 1.88 | 1.54 | 1.10 | 0.85 | 0.70 |
| | LOC51754 | | | | | | | | | | | |
| p1A8 | Lactate dehydrogenase | 224 | 0.24 | 1.07 | 1.09 | 1.00 | 0.70 | 0.40 | 0.52 | 0.50 | 0.66 | 0.92 |
| | A | | | | | | | | | | | |
| P1K9 | Lipocortin I | 402 | 0.46 | 4.09 | 1.33 | 1.17 | 1.07 | 0.31 | 1.20 | 0.54 | 0.23 | 0.12 |
| p1B1 | Metallothionein 1G | 244 | 1.21 | 0.60 | 3.14 | 1.49 | 1.00 | 2.04 | 0.46 | 1.54 | 0.27 | 0.51 |
| P1K23 | MYC | 404 | 5.98 | 1.00 | 2.50 | 2.41 | 4.11 | 1.91 | 0.64 | 2.17 | 0.32 | 0.44 |
| p1B20 | Osteopontin | 268 | 0.05 | 1.01 | 2.74 | 1.69 | 0.22 | 0.36 | 0.24 | 0.52 | 0.35 | 0.92 |
| p1B21 | Osteopontin | 268 | 0.22 | 1.73 | 2.09 | 1.87 | 0.58 | 0.76 | 0.52 | 0.65 | 0.44 | 1.00 |
| p1F17 | P8 protein (candidate | 330 | 1.18 | 0.17 | 1.27 | 1.47 | 0.55 | 1.79 | 1.00 | 0.97 | 0.92 | 1.33 |
| | of metastasis 1) | | | | | | | | | | | |
| p1C11 | polyubiquitin | 378 | 1.11 | 0.91 | 1.59 | 1.53 | 1.00 | 1.34 | 0.63 | 0.65 | 0.51 | 2.52 |
| p2B1 | PRAME | 88 | 1.13 | 8.86 | 5.08 | 9.57 | 1.15 | 18.20 | 1.63 | 1.64 | 0.96 | 0.96 |
| p1B5 | Proline 4-hydroxylase, | 350 | 0.50 | 0.52 | 1.02 | 3.18 | 1.94 | 1.31 | 0.79 | 0.89 | 0.72 | 1.62 |
| | alpha polypeptide II | | | | | | | | | | | |
| p1P14 | Semaphorin 4b | 92 | 0.80 | 4.26 | 0.86 | 1.01 | 1.22 | 1.88 | 1.33 | 1.73 | 1.48 | 2.26 |
| p1A6 | SLC2A5 | 312 | 0.52 | 5.07 | 0.89 | 2.12 | 1.48 | 0.68 | 1.21 | 0.55 | 1.37 | 0.87 |
| p1J17 | SLC6A1 | 438 | 0.11 | 0.39 | 0.85 | 1.42 | 0.80 | 3.56 | 3.08 | 1.17 | 4.08 | 1.00 |
| p1J18 | Synaptopodin | 440 | 0.07 | 0.31 | 0.99 | 1.44 | 0.68 | 3.03 | 3.51 | 1.40 | 4.54 | 1.29 |
| p1J15 | TERA protein | 442 | 0.68 | 2.14 | 0.80 | 1.47 | 1.00 | 2.36 | 2.97 | 2.74 | 3.14 | 0.97 |
| p1G11 | Tumor protein D52 | 302 | 0.20 | 1.49 | 0.51 | 1.07 | 0.83 | 1.00 | 1.66 | 1.82 | 1.90 | 2.62 |
| p1H18 | Ubiquitin specific | 158 | 0.73 | 2.13 | 0.91 | 1.76 | 0.82 | 2.47 | 2.56 | 2.04 | 2.53 | 1.00 |
| | protease 7 | | | | | | | | | | | |
| p1O20 | VEGF | 228 | 0.84 | 4.19 | 0.85 | 1.55 | 2.31 | 12.69 | 0.66 | 1.42 | 1.35 | 6.44 |

TABLE 19
Genes down-regulated in human tumors. Individual patients are denoted by the letters E,F,G,H and K.

| | | | Ovary | Ovary | Ovary | Ovary | Ovary | Ovary | Breast | Breast | Breast | Breast |
|---|---|---|---|---|---|---|---|---|---|---|---|---|
| | | Seq | nor | tum | nor | tum | nor | tum | nor | tum | nor | tum |
| Clone | Gene Name | ID | E | E | F | F | G | G | H | H | K | K |
| p1C3 | Activin A receptor, type I | 362 | 1.51 | 0.32 | 1.28 | 1.76 | 1.06 | 1.21 | 0.99 | 0.87 | 0.76 | 1.17 |
| p1B9 | adipophilin | 314 | 0.67 | 0.20 | 0.78 | 0.50 | 1.53 | 2.45 | 0.61 | 0.83 | 0.68 | 0.85 |
| p1K15 | Alpha-2-macroglobulin | 406 | 0.39 | 0.12 | 0.79 | 0.54 | 1.08 | 0.38 | 0.71 | 1.16 | 0.53 | 0.71 |
| p1G3 | B-cell translocation gene 1 | 318 | 2.01 | 0.64 | 1.15 | 1.13 | 1.69 | 0.58 | 1.39 | 1.13 | 1.66 | 0.78 |
| p1F14 | Butyrate response factor 1 | 328 | 2.85 | 0.94 | 1.86 | 2.31 | 1.46 | 0.79 | 1.36 | 1.00 | 0.86 | 1.09 |
| p1J23 | Calgranulin A | 448 | 0.43 | 0.90 | 1.00 | 0.91 | 11.60 | 0.59 | 12.02 | 1.60 | 23.70 | 0.80 |
| p1K2 | CFFM4 | 434 | 0.47 | 0.29 | 1.30 | 1.06 | 2.32 | 0.31 | 1.00 | 0.49 | 0.79 | 1.52 |
| p1J19 | CFFM4 | 434 | 0.45 | 0.29 | 1.24 | 1.00 | 2.06 | 0.35 | 1.42 | 0.56 | 0.76 | 1.44 |
| p2A23 | Chitinase 3-like 2 | 284 | 0.66 | 0.78 | 0.48 | 0.61 | 4.18 | 0.74 | 1.36 | 2.22 | 0.91 | 2.01 |
| p1N17 | COX-2 | 238 | 0.73 | 1.21 | 0.51 | 0.72 | 2.31 | 0.57 | 0.80 | 0.61 | 0.56 | 0.54 |
| p1C2 | CXCR4 | 332 | 0.29 | 0.91 | 1.03 | 1.41 | 2.43 | 2.80 | 2.71 | 0.95 | 1.81 | 0.59 |
| p1E3 | CYP1B1 | 138 | 0.32 | 0.06 | 0.45 | 1.47 | 1.05 | 0.16 | 1.00 | 0.38 | 1.20 | 0.16 |
| p1F16 | CYP1B1 | 326 | 0.60 | 0.17 | 0.67 | 2.30 | 1.65 | 0.24 | 1.00 | 0.55 | 1.82 | 0.24 |
| p1C8 | Dec1 | 372 | 3.93 | 0.66 | 1.85 | 1.10 | 1.37 | 0.56 | 0.94 | 0.53 | 0.87 | 5.55 |
| p1J10 | DNCLI2 | 460 | 1.00 | 0.98 | 1.28 | 1.17 | 1.66 | 0.50 | 2.02 | 1.51 | 0.98 | 0.90 |
| p1I23 | Ecotropic viral integration | 476 | 0.64 | 0.67 | 0.88 | 1.08 | 1.28 | 0.39 | 0.79 | 0.71 | 1.06 | 0.78 |
| | site 2A | | | | | | | | | | | |
| p1E4 | EST | 126 | 1.30 | 1.67 | 0.88 | 0.65 | 0.70 | 0.17 | 1.00 | 0.69 | 1.06 | 0.46 |
| p1H19 | EST | 196 | 0.71 | 1.52 | 0.80 | 0.99 | 1.08 | 1.17 | 1.99 | 1.70 | 3.34 | 0.83 |
| p1H4 | EST | 214 | 0.32 | 0.76 | 0.70 | 1.02 | 1.14 | 2.36 | 2.34 | 1.65 | 2.90 | 0.75 |
| p1H3 | EST | 216 | 0.10 | 0.38 | 0.78 | 1.14 | 0.88 | 2.49 | 2.26 | 1.18 | 3.08 | 0.89 |
| p1H15 | EST | 178 | 0.22 | 0.46 | 0.71 | 1.07 | 0.82 | 2.57 | 2.62 | 1.22 | 3.66 | 1.07 |
| p1H17 | EST | 172 | 0.65 | 2.20 | 1.00 | 1.62 | 0.95 | 2.64 | 2.95 | 2.32 | 2.98 | 0.93 |
| p1J11 | Fatty-acid-Coenzyme A | 466 | 0.83 | 0.23 | 0.68 | 1.38 | 1.05 | 0.44 | 1.42 | 0.85 | 1.00 | 1.00 |
| | ligase, long-chain 2 | | | | | | | | | | | |
| p1F24 | Glia-derived nexin | 298 | 3.17 | 0.93 | 3.24 | 0.85 | 1.26 | 0.80 | 1.64 | 1.89 | 1.25 | 0.96 |
| p1I24 | GRO1 | 486 | 0.82 | 2.23 | 1.55 | 3.98 | 3.19 | 0.52 | 0.50 | 0.55 | 0.31 | 0.52 |
| p1I19 | GRO2 | 482 | 1.97 | 2.36 | 3.67 | 4.14 | 11.31 | 1.00 | 1.29 | 1.48 | 0.64 | 0.70 |
| p1D1 | hypothetical protein | 24 | 1.20 | 0.40 | 1.82 | 1.82 | 0.73 | 0.68 | 0.81 | 1.03 | 1.28 | 0.96 |
| | FLJ10134 | | | | | | | | | | | |
| p1H21 | hypothetical protein | 164 | 1.00 | 0.22 | 0.97 | 0.49 | 1.73 | 0.72 | 1.35 | 0.75 | 2.85 | 1.76 |
| | FLJ13511 | | | | | | | | | | | |
| p1F2 | hypothetical protein | 4 | 2.41 | 0.61 | 1.20 | 0.63 | 1.40 | 0.39 | 0.66 | 0.73 | 0.53 | 0.63 |
| | FLJ20037 | | | | | | | | | | | |
| p1F23 | hypothetical protein | 290 | 1.27 | 0.85 | 0.90 | 1.03 | 1.82 | 0.55 | 0.90 | 0.92 | 0.84 | 0.94 |
| | LOC51014 | | | | | | | | | | | |
| p1E13 | hypothetical protein | 22 | 0.51 | 0.60 | 1.00 | 0.98 | 1.74 | 0.52 | 2.15 | 0.98 | 2.26 | 0.85 |
| | PRO0823 | | | | | | | | | | | |
| p1F3 | hypothetical protein | 334 | 2.75 | 0.91 | 1.26 | 1.39 | 1.42 | 0.61 | 1.67 | 1.96 | 1.13 | 1.02 |
| | XP_017131 | | | | | | | | | | | |
| p1B14 | Interleukin 8 | 252 | 2.96 | 0.36 | 4.71 | 1.04 | 8.45 | 1.00 | 0.36 | 0.37 | 0.54 | 0.37 |
| p1B16 | Interleukin 8 | 252 | 3.16 | 0.50 | 7.46 | 1.82 | 16.49 | 3.29 | 1.00 | 0.42 | 0.66 | 0.36 |
| p1B15 | Interleukin 8 | 252 | 3.43 | 0.54 | 7.25 | 1.69 | 11.81 | 3.46 | 0.94 | 0.75 | 0.67 | 0.88 |
| p1C13 | Jk-recombination signal | 382 | 1.94 | 0.55 | 1.48 | 1.17 | 0.54 | 0.50 | 0.54 | 0.80 | 0.51 | 0.57 |
| | binding protein | | | | | | | | | | | |
| p1K9 | Lipocortin I | 402 | 0.46 | 4.09 | 1.33 | 1.17 | 1.07 | 0.31 | 1.20 | 0.54 | 0.23 | 0.12 |
| p1E2 | Mannosidase, alpha, class | 122 | 0.87 | 0.40 | 1.00 | 0.88 | 2.02 | 0.50 | 1.51 | 0.67 | 1.54 | 1.63 |
| | 1A, member 1 | | | | | | | | | | | |
| p1A23 | Metallothionein 2A | 266 | 0.78 | 0.22 | 2.96 | 1.22 | 2.34 | 1.05 | 0.58 | 1.50 | 0.40 | 0.48 |
| p1G19 | Mitochondrion sequence | 208 | 1.00 | 1.74 | 1.17 | 1.08 | 0.99 | 1.69 | 2.16 | 1.67 | 2.10 | 0.58 |
| p1G18 | Mitochondrion sequence | 212 | 0.77 | 1.43 | 0.95 | 1.02 | 1.00 | 0.99 | 1.71 | 1.62 | 2.61 | 0.86 |
| p1K23 | MYC | 404 | 5.98 | 1.00 | 2.50 | 2.41 | 4.11 | 1.91 | 0.64 | 2.17 | 0.32 | 0.44 |
| p1J20 | Neuro-oncological ventral | 426 | 0.54 | 1.61 | 1.00 | 1.50 | 1.24 | 2.65 | 3.08 | 2.55 | 2.77 | 0.83 |
| | antigen 1 | | | | | | | | | | | |
| p1F17 | P8 protein (candidate of | 330 | 1.18 | 0.17 | 1.27 | 1.47 | 0.55 | 1.79 | 1.00 | 0.97 | 0.92 | 1.33 |
| | metastasis 1) | | | | | | | | | | | |
| p1B19 | plasminogen activator | 236 | 3.22 | 0.44 | 2.15 | 1.84 | 7.67 | 2.48 | 0.97 | 0.56 | 0.88 | 0.86 |
| | inhibitor, type 1 | | | | | | | | | | | |
| p1B18 | plasminogen activator | 236 | 1.53 | 0.34 | 1.27 | 1.28 | 2.90 | 1.44 | 0.52 | 0.84 | 0.79 | 0.75 |
| | inhibitor, type 1 | | | | | | | | | | | |
| p1K3 | Pleckstrin | 432 | 0.39 | 0.45 | 0.99 | 0.92 | 1.78 | 0.43 | 1.39 | 0.41 | 1.66 | 1.00 |
| p1B3 | Proline 4-hydroxylase, | 232 | 1.12 | 1.00 | 1.44 | 1.83 | 0.71 | 0.74 | 0.78 | 0.25 | 1.22 | 0.50 |
| | alpha polypeptide I | | | | | | | | | | | |
| p1F20 | Proline-rich protein with | 336 | 4.29 | 0.54 | 2.08 | 1.23 | 2.34 | 1.08 | 0.89 | 1.00 | 1.13 | 0.61 |
| | nuclear targeting signal | | | | | | | | | | | |
| p1B22 | Protease, serine, 11 | 356 | 3.86 | 0.86 | 9.76 | 2.40 | 1.17 | 0.95 | 1.18 | 0.88 | 0.79 | 1.00 |
| p1C10 | Regulator of G-protein | 376 | 0.26 | 0.12 | 1.09 | 1.10 | 1.93 | 0.14 | 0.42 | 0.12 | 0.44 | 1.00 |
| | signalling 1 | | | | | | | | | | | |
| p1P5 | SCYA2 | 396 | 2.43 | 0.63 | 3.14 | 1.39 | 2.20 | 0.61 | 1.14 | 0.98 | 0.82 | 0.89 |
| p1K8 | SCYA4 | 408 | 1.00 | 0.56 | 2.48 | 2.29 | 1.94 | 0.54 | 1.94 | 0.68 | 1.75 | 1.62 |
| p1I11 | SECIS binding protein 2 | 60 | 1.49 | 0.90 | 1.00 | 0.98 | 1.09 | 0.36 | 2.31 | 2.24 | 2.10 | 1.49 |
| p1I18 | Selectin L | 488 | 0.60 | 0.78 | 1.00 | 1.46 | 4.25 | 1.46 | 5.73 | 1.09 | 4.30 | 2.26 |
| p1D3 | Serine carboxypeptidase 1 | 96 | 0.76 | 0.09 | 0.91 | 0.98 | 0.76 | 0.50 | 0.98 | 1.00 | 1.13 | 0.95 |
| p1J17 | SLC6A1 | 438 | 0.11 | 0.39 | 0.85 | 1.42 | 0.80 | 3.56 | 3.08 | 1.17 | 4.08 | 1.00 |
| p1F7 | Spectrin, beta, non- | 16 | 2.52 | 0.48 | 2.04 | 1.14 | 1.65 | 0.95 | 2.30 | 1.90 | 0.91 | 0.91 |
| | erythrocytic 1 | | | | | | | | | | | |
| p1J18 | Synaptopodin | 440 | 0.07 | 0.31 | 0.99 | 1.44 | 0.68 | 3.03 | 3.51 | 1.40 | 4.54 | 1.29 |
| p1J15 | TERA protein | 442 | 0.68 | 2.14 | 0.80 | 1.47 | 1.00 | 2.36 | 2.97 | 2.74 | 3.14 | 0.97 |
| p1K4 | TSC-22 | 444 | 2.92 | 0.46 | 1.40 | 2.19 | 0.85 | 1.05 | 0.56 | 1.12 | 0.35 | 0.88 |

TABLE 20
Genes up-regulated in response to TNFα

| | | | Cytokine/% Oxygen | | | |
|---|---|---|---|---|---|---|
| | | Seq | none | none | TNF | TNF |
| Clone | Gene Name | ID | 20 | 0.1 | 20 | 0.1 |
| p1C14 | Abstrakt | 384 | 1 | 7.89 | 4.63 | 7.87 |
| p1D13 | Adenylate kinase 3 | 78 | 1 | 0.85 | 2.36 | 2.47 |
| p1A22 | Adenylate kinase 3 | 264 | 1 | 1.53 | 2.69 | 3.72 |
| p1B8 | adipophilin | 314 | 1 | 17.1 | 2.27 | 8.81 |
| p1B7 | adipophilin | 314 | 1 | 13.4 | 2.17 | 5.86 |
| p1A19 | Aldolase C | 260 | 1 | 6.61 | 2.57 | 6.31 |
| p1N17 | COX-2 | 238 | 1 | 1.04 | 0.91 | 2.24 |
| p1C1 | CXCR4 | 332 | 1 | 5.42 | 2.21 | 5.22 |
| p1F4 | CYP1 | 340 | 1 | 2.86 | 3.43 | 6.26 |
| p1E3 | CYP1B1 | 138 | 1 | 0.33 | 2.12 | 1.14 |
| p1F16 | CYP1B1 | 326 | 1 | 0.45 | 1.93 | 1.19 |
| p2L23 | endothelin 1 | 398 | 1 | 0.95 | 2.74 | 2.41 |
| p1A14 | Enolase 1 | 258 | 1 | 9.98 | 7.22 | 11.78 |
| p1A11 | GAPDH | 254 | 1 | 7.87 | 3.60 | 5.90 |
| p1C6 | Glucose phosphate | 368 | 1 | 5.18 | 2.58 | 3.61 |
| | isomerase | | | | | |
| p1D9 | hypothetical protein | 28 | 1 | 2.37 | 2.48 | 3.16 |
| | DKFZP564D116 | | | | | |
| p1F5 | hypothetical protein | 12 | 1 | 3.84 | 2.05 | 3.78 |
| | FLJ20281 | | | | | |
| p1B23 | Interleukin 1 receptor | 358 | 1 | 3.46 | 3.43 | 5.35 |
| | antagonist | | | | | |
| p1B14 | Interleukin 8 | 252 | 1 | 5.52 | 16.8 | 56.8 |
| p1B16 | Interleukin 8 | 252 | 1 | 2.55 | 9.64 | 23.3 |
| p1B15 | Interleukin 8 | 252 | 1 | 3.37 | 10.4 | 28.1 |
| p1C13 | Jk-recombination signal | 382 | 1 | 5.82 | 4.77 | 8.75 |
| | binding protein | | | | | |
| p1A8 | Lactate dehydrogenase A | 224 | 1 | 24.8 | 4.08 | 15.1 |
| p1A13 | Phosphoglycerate kinase 1 | 256 | 1 | 7.29 | 2.73 | 4.65 |
| p1B19 | Plasminogen activator | 236 | 1 | 3.78 | 2.63 | 9.41 |
| | inhibitor, type 1 | | | | | |
| p1B18 | Plasminogen activator | 236 | 1 | 4.92 | 2.23 | 6.55 |
| | inhibitor, type 1 | | | | | |
| p1C11 | Polyubiquitin | 378 | 1 | 2.80 | 2.06 | 3.03 |
| p1B4 | Proline 4-hydroxylase, | 350 | 1 | 6.15 | 3.09 | 5.80 |
| | alpha polypeptide II | | | | | |
| p1F20 | Proline-rich protein with | 336 | 1 | 4.69 | 2.18 | 6.45 |
| | nuclear targeting signal | | | | | |
| p1I20 | SCYA3L | 470 | 1 | 0.77 | 3.97 | 3.61 |
| p1K8 | SCYA4 | 408 | 1 | 0.81 | 9.65 | 9.63 |
| p1D3 | Serine carboxypeptidase 1 | 96 | 1 | 3.74 | 2.37 | 3.55 |
| p1A2 | SLC2A3 | 248 | 1 | 16.0 | 2.68 | 15.5 |
| p1F22 | Sorting nexin 9 | 320 | 1 | 0.66 | 1.26 | 1.63 |
| p1B10 | Stearoyl-CoA desaturase | 352 | 1 | 5.04 | 3.05 | 6.95 |
| p1B17 | Tissue factor | 226 | 1 | 3.69 | 2.74 | 6.03 |
| p1A20 | Triosephosphate isomerase | 262 | 1 | 16.1 | 8.30 | 16.2 |
| | 1 | | | | | |
| p1E14 | unknown mRNA | 98 | 1 | 3.30 | 3.03 | 3.26 |
| | (schizophrenia-linked) | | | | | |

TABLE 21
Genes down-regulated in response to TNFα

| | | | Cytokine/% Oxygen | | | |
|---|---|---|---|---|---|---|
| | | | none | none | TNF | TNF |
| Clone | Gene Name | Seq ID | 20 | 0.1 | 20 | 0.1 |
| p1E5 | Hepcidin antimicrobial | 142 | 1 | 1.50 | 0.19 | 0.70 |
| | peptide | | | | | |
| p1H2 | Fatty acid binding protein 5 | 210 | 1 | 0.72 | 0.38 | 0.46 |
| p1P5 | SCYA2 | 396 | 1 | 0.29 | 0.45 | 0.26 |
| p1J5 | SCYA7 | 464 | 1 | 0.89 | 0.46 | 0.49 |

TABLE 22
Genes up-regulated in response to IL-17

| | | | Cytokine/% Oxygen | | | |
|---|---|---|---|---|---|---|
| | | | none | none | IL-17 | IL-17 |
| Clone | Gene Name | Seq ID | 20 | 0.1 | 20 | 0.1 |
| p1J11 | Fatty-acid-Coenzyme A | 466 | 1 | 0.55 | 1.63 | 1.28 |
| | ligase, long-chain 2 | | | | | |
| p1A11 | GAPDH | 254 | 1 | 0.78 | 2.60 | 1.84 |
| p1C6 | Glucose phosphate | 368 | 1 | 0.84 | 2.14 | 1.43 |
| | isomerase | | | | | |
| p1I24 | GRO1 | 486 | 1 | 1.02 | 2.29 | 1.28 |
| p1I19 | GRO2 | 482 | 1 | 1.02 | 2.26 | 1.43 |
| p1B16 | Interleukin 8 | 252 | 1 | 1.77 | 9.52 | 12.2 |
| p1B15 | Interleukin 8 | 252 | 1 | 1.54 | 7.36 | 9.71 |
| p1B14 | Interleukin 8 | 252 | 1 | 1.50 | 9.34 | 7.13 |
| p1P5 | SCYA2 | 396 | 1 | 0.24 | 2.12 | 0.58 |
| p1K8 | SCYA4 | 408 | 1 | 0.44 | 2.48 | 0.83 |

TABLE 23
Genes down-regulated in response to IL-17

| | | | Cytokine/% Oxygen | | | |
|---|---|---|---|---|---|---|
| | | Seq | none | none | IL-17 | IL-17 |
| Clone | Gene Name | ID | 20 | 0.1 | 20 | 0.1 |
| p1H8 | ABL | 182 | 1 | 1.08 | 0.08 | 0.09 |
| p1J22 | Neutral | 428 | 1 | 1.21 | 0.13 | 0.11 |
| | sphingomyelinase | | | | | |
| | (N-SMase) activation | | | | | |
| | associated factor | | | | | |
| p1K14 | Keratin 6B | 422 | 1 | 1.27 | 0.15 | 0.15 |
| p1J6 | hypothetical protein | 40 | 1 | 1.28 | 0.22 | 0.23 |
| | FLJ10206 | | | | | |
| p1I12 | hypothetical protein | 152 | 1 | 1.20 | 0.32 | 0.30 |
| | MGC4549 | | | | | |
| p1E15 | cDNA YI27F12 | 108 | 1 | 1.58 | 0.21 | 0.56 |
| p2A14 | EST | 446 | 1 | 1.10 | 0.56 | 0.40 |
| p1G24 | Glycogen synthase 1 | 276 | 1 | 1.45 | 0.34 | 0.65 |
| p1C16 | Decidual protein | 388 | 1 | 1.09 | 0.73 | 0.51 |
| | induced by progesterone | | | | | |
| p1D8 | Hypoxia-inducible | 272 | 1 | 1.30 | 0.44 | 0.65 |
| | protein 2 | | | | | |
| p1B18 | Plasminogen activator | 236 | 1 | 1.10 | 0.49 | 0.78 |
| | inhibitor, type 1 | | | | | |
| p1H4 | EST | 214 | 1 | 1.13 | 0.49 | 0.58 |

TABLE 24
Genes up-regulated in response to IL-15

| | | | Cytokine/% Oxygen | | | |
|---|---|---|---|---|---|---|
| | | Seq | none | none | IL-15 | IL-15 |
| Clone | Gene Name | ID | 20 | 0.1 | 20 | 0.1 |
| p1A19 | Aldolase C | 260 | 1 | 0.50 | 0.35 | 1.30 |
| p1J16 | cDNA: FLJ23019 fis, | 454 | 1 | 0.80 | 5.76 | 7.27 |
| | clone LNG00916 | | | | | |
| p1D19 | EST | 144 | 1 | 1.00 | 2.27 | 1.39 |
| p1J11 | Fatty-acid-Coenzyme A | 466 | 1 | 0.55 | 1.64 | 1.29 |
| | ligase, long-chain 2 | | | | | |
| p1A11 | GAPDH | 254 | 1 | 0.78 | 0.52 | 4.32 |
| p1C6 | Glucose phosphate | 368 | 1 | 0.84 | 0.57 | 3.13 |
| | isomerase | | | | | |
| p1H5 | hypothetical protein | 206 | 1 | 0.94 | 2.37 | 1.59 |
| | FLJ22690 | | | | | |
| p1A23 | Metallothionein 2A | 266 | 1 | 1.41 | 1.26 | 3.08 |
| p1P5 | SCYA2 | 396 | 1 | 0.24 | 4.51 | 1.37 |
| p1J5 | SCYA7 | 464 | 1 | 0.66 | 3.27 | 1.61 |
| p1I21 | SCYA8 | 480 | 1 | 0.37 | 3.77 | 1.55 |
| p1I7 | Uridine 5′ | 50 | 1 | 0.84 | 4.98 | 3.61 |
| | monophosphate | | | | | |
| | hydrolase 1 | | | | | |

TABLE 25
Genes down-regulated in response to IL-15

| | | | Cytokine/% Oxygen | | | |
|---|---|---|---|---|---|---|
| | | Seq | none | none | IL-15 | IL-15 |
| Clone | Gene Name | ID | 20 | 0.1 | 20 | 0.1 |
| p1H8 | ABL | 182 | 1 | 1.08 | 1.22 | 0.09 |
| p1C14 | Abstrakt | 384 | 1 | 0.69 | 0.40 | 1.24 |
| p1B8 | Adipophilin | 314 | 1 | 1.08 | 0.23 | 1.42 |
| p1B7 | Adipophilin | 314 | 1 | 1.10 | 0.30 | 2.02 |
| p1B6 | Adipophilin | 314 | 1 | 0.98 | 0.37 | 2.39 |
| p1B9 | Adipophilin | 314 | 1 | 1.46 | 0.41 | 1.66 |
| p1A19 | Aldolase C | 260 | 1 | 0.50 | 0.35 | 1.30 |
| p1C7 | D123 | 370 | 1 | 0.53 | 0.47 | 0.90 |
| p1H6 | EST | 192 | 1 | 1.46 | 1.95 | 0.67 |
| p2A14 | EST | 446 | 1 | 1.10 | 1.08 | 0.51 |
| p1G24 | Glycogen synthase 1 | 276 | 1 | 1.45 | 0.88 | 0.55 |
| p1J6 | hypothetical protein | 40 | 1 | 1.28 | 1.28 | 0.18 |
| | FLJ10206 | | | | | |
| p1I12 | hypothetical protein | 152 | 1 | 1.20 | 1.64 | 0.28 |
| | MGC4549 | | | | | |
| p1D8 | Hypoxia-inducible | 272 | 1 | 1.30 | 1.25 | 0.62 |
| | protein 2 | | | | | |
| p1K14 | Keratin 6B | 422 | 1 | 1.27 | 1.48 | 0.11 |
| p1A8 | Lactate dehydrogenase A | 224 | 1 | 1.31 | 0.48 | 2.29 |
| p1A9 | Lactate dehydrogenase A | 224 | 1 | 1.95 | 0.49 | 1.83 |
| p1J22 | Neutral | 428 | 1 | 1.21 | 1.51 | 0.11 |
| | sphingomyelinase | | | | | |
| | (N-SMase) activation | | | | | |
| | associated factor | | | | | |
| p1B4 | Proline 4-hydroxylase, | 350 | 1 | 0.81 | 0.50 | 1.34 |
| | alpha polypeptide II | | | | | |
| p1A20 | Triosephosphate | 262 | 1 | 0.81 | 0.36 | 1.22 |
| | isomerase 1 | | | | | |

TABLE 26
cross-references all protein and nucleotide sequences (SEQ ID NOS) that are referenced
herein to accession numbers in public databases available as of 8.12.00.

| | Hypoxia | PROTEIN | | NUCLEOTIDE | |
|---|---|---|---|---|---|
| TITLE | response | SEQ ID | ACCESSION | SEQ ID | ACCESSION |
| cDNA FLJ13611 fis, clone PLACE1010802 | Increase | 1 | BAB14633 | 2 | AK023673 |
| hypothetical protein FLJ20037 | Increase | 3 | BAA90903 | 4 | AK000044 |
| hypothetical protein DKFZp434P0116 | Increase | 5 | CAB70863 | 6 | AL137661 |
| KIAA0212 | Increase | 7 | BAA13203 | 8 | D86967 |
| KIAA0914 | Increase | 9 | BAA74937 | 10 | AB020721 |
| hypothetical protein FLJ20281 | Increase | 11 | NP_060212 | 12 | NM_017742 |
| KIAA0876 | Increase | 13 | BAA74899 | 14 | AB020683 |
| cDNA FLJ13700 fis, clone PLACE2000216 | Increase | 15 | (nearest = Q0108 | 16 | AK023762 |
| | | | 2) | | |
| DKFZP586G1122 protein | Increase | 17 | CAB55938 | 18 | AL117462 |
| Putative zinc finger protein LOC55818 | Increase | 19 | AAF67005 | 20 | AF155648 |
| hypothetical protein PRO0823 | Increase | 21 | AAF71073 | 22 | AF116653 |
| hypothetical protein FLJ10134 | Increase | 23 | BAA91458 | 24 | AK000996 |
| hypothetical protein FLJ20500 | Increase | 25 | BAA91214 | 26 | AK000507 |
| DKFZP564D116 protein | Increase | 27 | CAB43242 | 28 | AL050022 |
| KIAA1376 protein | Increase | 29 | BAA92614 | 30 | AB037797 |
| hypothetical protein KIAA0127 | Increase | 31 | BAA09476 | 32 | D50917 |
| hypothetical protein FLJ20308 | Increase | 33 | BAA91078 | 34 | AL137263 |
| hypothetical nuclear factor SBBI22 | Repression | 35 | NP_065128 | 36 | NM_020395 |
| DKFZP434I116 protein | Repression | 37 | CAB55922 | 38 | AL117434 |
| hypothetical prot. FLJ10206 | Repression | 39 | NP_060495 | 40 | NM_018025 |
| hypothetical protein FLJ10815 | Repression | 41 | BAA91830 | 42 | AK001677 |
| hypothetical protein FLJ11100 | Repression | 43 | BAA92003 | 44 | AK001962 |
| hypothetical protein FLJ2064 | Repression | 45 | NP_060387 | 46 | NM_017917 |
| hypothetical protein HSPC111 | Repression | 47 | NP_057475 | 48 | NM_016391 |
| hypothetical protein LOC51251 | Repression | 49 | NP_057573 | 50 | NM_016489 |
| KIAA0014 | Repression | 51 | BAA04946 | 52 | D25216 |
| hypothetical protein HSPC196 | Repression | 53 | NP_057548 | 54 | NM_016464 |
| hypothetical protein FLJ11296 | Repression | 55 | BAA92115 | 56 | AK002158 |
| hypothetical protein bA395L14 | Repression | 57 | CAB62980 | 58 | AL022311 |
| cDNA FLJ13016 fis, clone NT2RP3000624 | Repression | 59 | BAB14393 | 60 | AK023078 |
| cDNA DKFZp586H0324 clone DKFZp586H0324 | Increase | 61 | none | 62 | AL110163 |
| Clone 23785 | Increase | 63 | none | 64 | AF035307 |
| cDNA DKFZp586E1624 | Increase | 65 | none | 66 | AL110152 |
| cDNA FLJ14162 fis, clone NT2RM4002504 | Increase | 67 | none | 68 | AK024224 |
| cDNA DKFZp434E1723 (clone DKFZp434E1723) | Increase | 69 | none | 70 | AL137473 |
| cDNA FLJ11041 fis, clone PLACE1004405 | Increase | 71 | none | 72 | AK001903 |
| cDNA FLJ10433 fis NT2RP1000478 | Increase | 73 | none | 74 | AK001295 |
| cDNA DKFZp434O071 | Increase | 75 | none | 76 | AF125392 |
| cDNA FLJ23313 fis, clone HEP11919 | Increase | 77 | none | 78 | AK026966 |
| ESTs | Increase | 79 | none | 80 | R62339 |
| ESTs | Increase | 81 | none | 82 | AA489477 |
| ESTs | Increase | 83 | none | 84 | R06601 |
| ESTs | Increase | 85 | none | 86 | R00332 |
| ESTs | Increase | 87 | none | 88 | AA463469 |
| ESTs | Increase | 89 | none | 90 | H56028 |
| ESTs | Increase | 91 | none | 92 | AA293300 |
| ESTs | Increase | 93 | none | 94 | AW250104 |
| ESTs | Increase | 95 | none | 96 | BE382614 |
| ESTs | Increase | 97 | none | 98 | H59618 |
| ESTs | Increase | 99 | none | 100 | AA449703 |
| ESTs | Increase | 101 | none | 102 | AA521311 |
| ESTs | Increase | 103 | none | 104 | W69170 |
| ESTs | Increase | 105 | none | 106 | R51835 |
| ESTs | Increase | 107 | none | 108 | H87770 |
| ESTs | Increase | 109 | none | 110 | R69248 |
| ESTs | Increase | 111 | none | 112 | T68844 |
| ESTs | Increase | 113 | none | 114 | AA454177 |
| ESTs | Increase | 115 | none | 116 | AA026562 |
| ESTs | Increase | 117 | none | 118 | T73780 |
| ESTs | Increase | 119 | none | 120 | AA401496 |
| ESTs | Increase | 121 | none | 122 | AA489636 |
| ESTs | Increase | 123 | none | 124 | AA446361 |
| ESTs | Increase | 125 | none | 126 | AA931411 |
| ESTs | Increase | 127 | none | 128 | R24223 |
| ESTs | Increase | 129 | none | 130 | R22252 |
| ESTs | Increase | 131 | none | 132 | AA612751 |
| ESTs | Increase | 133 | none | 134 | AW964331 |
| ESTs | Increase | 135 | none | 136 | AI018611 |
| ESTs | Increase | 137 | none | 138 | AA451886 |
| ESTs | Increase | 139 | none | 140 | R06520 |
| ESTs | Increase | 141 | none | 142 | T48278 |
| ESTs | Increase | 143 | none | 144 | R68736 |
| cDNA FLJ14028 fis, clone HEMBA1003838 | Repression | 145 | none | 146 | AK024090 |
| cDNA DKFZp564D016 (clone DKFZp564D016) | Repression | 147 | none | 148 | AL050021 |
| cDNA FLJ11302 fis, clone PLACE1009971 | Repression | 149 | none | 150 | AK002164 |
| NEDO FLJ10309 fis cl NT2RM2000287 | Repression | 151 | none | 152 | AK001171 |
| Sequence from clone RP11-394O2 on ch 20 | Repression | 153 | none | 154 | AK022731 |
| ESTs | Repression | 155 | none | 156 | AA420992 |
| ESTs | Repression | 157 | none | 158 | AA693797 |
| ESTs | Repression | 159 | none | 160 | AA456437 |
| ESTs | Repression | 161 | none | 162 | AA429367 |
| ESTs | Repression | 163 | none | 164 | AA434382 |
| ESTs | Repression | 165 | none | 166 | AA664228 |
| ESTs | Repression | 167 | none | 168 | R44397 |
| ESTs | Repression | 169 | none | 170 | AA923509 |
| ESTs | Repression | 171 | none | 172 | W87747 |
| ESTs | Repression | 173 | none | 174 | AA973568 |
| ESTs | Repression | 175 | none | 176 | T98529 |
| ESTs | Repression | 177 | none | 178 | AA022679 |
| ESTs | Repression | 179 | none | 180 | H17921 |
| ESTs | Repression | 181 | none | 182 | R00766 |
| ESTs | Repression | 183 | none | 184 | W91958 |
| ESTs | Repression | 185 | none | 186 | R63694 |
| ESTs | Repression | 187 | none | 188 | AA425386 |
| ESTs | Repression | 189 | none | 190 | AA909912 |
| ESTs | Repression | 191 | none | 192 | T99032 |
| ESTs | Repression | 193 | none | 194 | H52503 |
| ESTs | Repression | 195 | none | 196 | AA127017 |
| ESTs | Repression | 197 | none | 198 | R38647 |
| ESTs | Repression | 199 | none | 200 | T87233 |
| ESTs | Repression | 201 | none | 202 | AA130351 |
| ESTs | Repression | 203 | none | 204 | H49601 |
| ESTs | Repression | 205 | none | 206 | AA598952 |
| ESTs | Repression | 207 | none | 208 | AA991868 |
| ESTs | Repression | 209 | none | 210 | T60111 |
| ESTs | Repression | 211 | none | 212 | AA897090 |
| ESTs | Repression | 213 | none | 214 | AA679939 |
| ESTs | Repression | 215 | none | 216 | AA630167 |
| BCL2/adenovirus E1B 19kD-interacting protein 3-like | Increase | 217 | NP_004322 | 218 | NM_004331 |
| Solute carrier family 2, member 1 | Increase | 219 | NP_006507 | 220 | NM_006516 |
| PDGF beta | Increase | 221 | NP_002599 | 222 | NM_002608 |
| lactate dehydrogenase A | Increase | 223 | NP_005557 | 224 | NM_005566 |
| Tissue factor | Increase | 225 | NP_001984 | 226 | NM_001993 |
| Vascular endothelial growth factor | Increase | 227 | NP_003367 | 228 | NM_003376 |
| RTP/NDRG1 | Increase | 229 | NP_006087 | 230 | NM_006096 |
| Procollagen-proline 4-hydroxylase alpha 1 | Increase | 231 | NP_000908 | 232 | NM_000917 |
| BCL2/adenovirus E1B-interacting protein 3 | Increase | 233 | NP_004043 | 234 | NM_004052 |
| Plasminogen activator inhibitor, type I | Increase | 235 | AAA60003 | 236 | M16006 |
| Cyclooxygenase 2 | Increase | 237 | AAA57317 | 238 | U04636 |
| Metallothionein 1H | Increase | 239 | CAA46046 | 240 | X64834 |
| Metallothionein 1L | Increase | 241 | P80297 | 242 | AJ011772 |
| Metallothionein-IG | Increase | 243 | AAA59873 | 244 | J03910 |
| Metallothionein 1E (functional) | Increase | 245 | AAA59587 | 246 | M10942 |
| Solute carrier family 2, member 3 | Increase | 247 | AAB61083 | 248 | M20681 |
| Hexokinase 2 | Increase | 249 | CAA86511 | 250 | Z46376 |
| Interleukin 8 | Increase | 251 | CAA68742 | 252 | Y00787 |
| Glyceraldehyde-3-phosphate dehydrogenase | Increase | 253 | NP_002037 | 254 | NM_002046 |
| Phosphoglycerate kinase 1 | Increase | 255 | NP_000282 | 256 | NM_000291 |
| Enolase 1 | Increase | 257 | NP_001419 | 258 | NM_001428 |
| aldolase C, fructose-bisphosphate (ALDOC) | Increase | 259 | NP_005156 | 260 | NM_005165 |
| Triosephosphate isomerase 1 (TPI1) | Increase | 261 | NP_000356 | 262 | NM_000365 |
| Adenylate kinase 3 (AK3) | Increase | 263 | NP_037542 | 264 | NM_013410 |
| Metallothionein-2a | Increase | 265 | AAA59583 | 266 | J00271 |
| Osteopontin | Increase | 267 | CAA31984 | 268 | X13694 |
| Granulin | Increase | 269 | AAA58617 | 270 | AK000607 |
| Hypoxia-inducible protein 2 | Increase | 271 | NP_037464 | 272 | NM_013332 |
| Enolase 2, (gamma, neuronal) | Increase | 273 | NP_001966 | 274 | NM_001975 |
| Glycogen synthase 1 (muscle) | Increase | 275 | AAB60385 | 276 | U32573 |
| Activated leucocyte cell adhesion molecule | Increase | 277 | NP_001618 | 278 | NM_001627 |
| MAX-interacting protein 1 | Increase | 279 | NP_005953 | 280 | NM_005962 |
| Nuclear receptor co-repressor | Increase | 281 | NP_006302 | 282 | NM_006311 |
| Chitinase 3-like 2 | Increase | 283 | AAC50597 | 284 | U49835 |
| BACH1 transcription factor | Increase | 285 | NP_001177 | 286 | NM_001186 |
| Phosphoglucomutase 1 | Increase | 287 | NP_002624 | 288 | NM_002633 |
| CGI-109 protein | Increase | 289 | AAD34104 | 290 | AF151867 |
| SAP30 | Increase | 291 | NP_003855 | 292 | NM_003864 |
| ATP-binding cassette transporter-1 | Increase | 293 | NP_005493 | 294 | NM_005502 |
| SEC24 protein | Increase | 295 | CAA10334 | 296 | AJ131244 |
| Trinucleotide repeat containing 3 | Increase | 297 | NP_005869 | 298 | NM_005878 |
| Post-synaptic density protein 95 | Increase | 299 | AAC52113 | 300 | U83192 |
| Tumor protein D52 | Increase | 301 | NP_005070 | 302 | NM_005079 |
| Cyclin-dependent kinase inhibitor p27kip1 | Increase | 303 | NP_004055 | 304 | NM_004064 |
| phosphoinositide-3-kinase, catalytic, beta | Increase | 305 | NP_006210 | 306 | NM_006219 |
| Solute carrier family 5, member 3 | Increase | 307 | NP_008864 | 308 | NM_006933 |
| PSCDBP | Increase | 309 | NP_004279 | 310 | NM_004288 |
| Solute carrier family 2, member 5 | Increase | 311 | AAA52570 | 312 | M55531 |
| Adipophilin | Increase | 313 | NP_001113 | 314 | NM_001122 |
| Early development regulator 2 | Increase | 315 | NP_004418 | 316 | NM_004427 |
| B-cell translocation gene 1, | Increase | 317 | NP_001722 | 318 | NM_001731 |
| SH3PX1 | Increase | 319 | NP_057308 | 320 | NM_016224 |
| Cyclin G2 | Increase | 321 | NP_004345 | 322 | NM_004354 |
| NAG-5 protein | Increase | 323 | NP_057530 | 324 | NM_016446 |
| Cytochrome P450 IB1 (dioxin-inducible) | Increase | 325 | NP_000095 | 326 | NM_000104 |
| Butyrate response factor 1 | Increase | 327 | NP_004917 | 328 | NM_004926 |
| p8 protein (candidate of metastasis 1) | Increase | 329 | NP_036517 | 330 | NM_012385 |
| chemokine (C-X-C motif), receptor 4 (CXCR4) | Increase | 331 | NP_003458 | 332 | NM_003467 |
| solute carrier family 16, member 6 | Increase | 333 | AAC52014 | 334 | U79745 |
| Proline-rich protein with nuclear targeting signal (B4-2) | Increase | 335 | NP_006804 | 336 | NM_006813 |
| RNA helicase-related protein | Increase | 337 | AAC32396 | 338 | AF083255 |
| Cytochrome P450, subfamily XXVIIB, polypeptide 1 | Increase | 339 | BAA22656 | 340 | AB005989 |
| SHB adaptor protein | Increase | 341 | CAA53091 | 342 | X75342 |
| Papillomavirus regulatory factor (PRF-1) | Increase | 343 | NP_061130 | 344 | NM_018660 |
| SLC31A2/hCTR1 | Increase | 345 | NP_001851 | 346 | NM_001860 |
| UDP-glucose pyrophosphorylase 2 (UGP2) | Increase | 347 | NP_006750 | 348 | NM_006759 |
| Proline 4-hydroxylase, alpha polypeptide II | Increase | 349 | NP_004190 | 350 | NM_004199 |
| Stearoyl-CoA desaturase | Increase | 351 | BAA93510 | 352 | AB032261 |
| Diacylglycerol kinase, zeta | Increase | 353 | NP_003637 | 354 | NM_003646 |
| Serine protease 11 | Increase | 355 | BAA13322 | 356 | Y07921 |
| IL-1 receptor antagonist, alternatively spliced forms | Increase | 357 | AAB92268, | 358 | U65590 |
| | | | AAB92269, | | |
| | | | AAB92270 | | |
| NS1-binding protein | Increase | 359 | NP_006460 | 360 | NM_006469 |
| Activin A receptor type I | Increase | 361 | NP_001096 | 362 | NM_001105 |
| FGF receptor activating protein 1 (FRAG1) | Increase | 363 | AAF19156 | 364 | AF159621 |
| Galectin-8 | Increase | 365 | AAF19370 | 366 | AF193806 |
| Glucose 6-phosphate isomerase | Increase | 367 | NP_000166 | 368 | NM_000175 |
| D123 protein | Increase | 369 | AAC34738 | 370 | U27112 |
| Dec1. | Increase | 371 | NP_003661 | 372 | NM_003670 |
| Rab-8b | Increase | 373 | NP_057614 | 374 | NM_016530 |
| BL34 | Increase | 375 | AAB26289 | 376 | S59049 |
| Polyubiquitin UbC | Increase | 377 | BAA23632 | 378 | AB009010 |
| Integrin alpha 5 | Increase | 379 | NP_002196 | 380 | NM_002205 |
| Jk-recombination signal binding protein | Increase | 381 | AAA60258 | 382 | L07872 |
| DEAD-box protein abstrakt | Increase | 383 | NP_057306 | 384 | NM_016222 |
| High mobility group 2 protein | Increase | 385 | AAA58659 | 386 | M83665 |
| Decidual protein induced by progesterone | Increase | 387 | NP_008952 | 388 | NM_007021 |
| GM2 ganglioside activator protein. | Increase | 389 | CAA43993, | 390 | X62078 |
| | | | CAA43994 | | |
| CCR4 associated factor 1 (CAF1) | Increase | 391 | AAD02685 | 392 | AF053318 |
| Nucleoside phosphorylase | Repression | 393 | NP_000261 | 394 | NM_000270 |
| Monocyte chemotactic protein 1 | Repression | 395 | NP_002973 | 396 | NM_002982 |
| Endothelin 1 | Repression | 397 | NP_001946 | 398 | NM_001955 |
| Heat shock 70kD protein 4 | Repression | 399 | AAA02807 | 400 | L12723 |
| Annexin AI | Repression | 401 | NP_000691 | 402 | NM_000700 |
| p67 myc protein | Repression | 403 | CAA25105, | 404 | X00364 |
| | | | CAA25106 | | |
| Alpha-2-macroglobulin | Repression | 405 | NP_000005 | 406 | NM_000014 |
| Macrophage inflammatory protein 1b | Repression | 407 | NP_002975 | 408 | NM_002984 |
| Sex hormone-binding globulin | Repression | 409 | NP_001031 | 410 | NM_001040 |
| ATP-binding cassette, sub-family E (OABP), member 1 | Repression | 411 | NP_002931 | 412 | NM_002940 |
| Chaperonin/Tcp zeta 1 | Repression | 413 | NP_001753 | 414 | NM_001762 |
| Colony stimulating factor 1 (macrophage) | Repression | 415 | AAA59573 | 416 | M27087 |
| Dendritic cell protein (GA17) | Repression | 417 | NP_006351 | 418 | NM_006360 |
| G protein-coupled receptor 44 | Repression | 419 | NP_004769 | 420 | NM_004778 |
| Keratin 6A | Repression | 421 | NP_005545 | 422 | NM_005554 |
| lymphocyte adaptor protein | Repression | 423 | NP_005466 | 424 | NM_005475 |
| Neuro-oncological ventral antigen 1 | Repression | 425 | AAA16022 | 426 | U04840 |
| N-SMase/FAN | Repression | 427 | CAA65405 | 428 | X96586 |
| Peptidylprolyl isomerase F (cyclophilin F) | Repression | 429 | NP_005720 | 430 | NM_005729 |
| PLECKSTRIN | Repression | 431 | NP_002655 | 432 | NM_002664 |
| High affinity immunoglobulin epsilon receptor beta | Repression | 433 | AAF17243 | 434 | AF201951 |
| Ribosomal protein L44 | Repression | 435 | NP_000992 | 436 | NM_001001 |
| Solute carrier family 6 No 1 | Repression | 437 | NP_003033 | 438 | NM_003042 |
| Synaptopodin | Repression | 439 | NP_009217 | 440 | NM_007286 |
| TERA protein | Repression | 441 | AAF87322 | 442 | AF212220 |
| TGF beta-stimulated protein TSC-22 | Repression | 443 | NP_006013 | 444 | NM_006022 |
| Tubulin, beta, 2 | Repression | 445 | NP_006079 | 446 | NM_006088 |
| Calgranulin A | Repression | 447 | NP_002955 | 448 | NM_002964 |
| Replication factor C (145 KDa) | Repression | 449 | NP_002904 | 450 | NM_002913 |
| Signal recognition particle 19 kD protein | Repression | 451 | NP_003126 | 452 | NM_003135 |
| Transcription factor SUPT3H | Repression | 453 | NP_003590 | 454 | NM_003599 |
| Proteasome component C9 | Repression | 455 | NP_002780 | 456 | NM_002789 |
| Maf-related leucine zipper homolog | Repression | 457 | NP_005452 | 458 | NM_005461 |
| dynein, cytoplasmic, light intermediate polypeptide 2 | Repression | 459 | NP_006132 | 460 | NM_006141 |
| Heterochromatin-like protein 1 | Repression | 461 | NP_057671 | 462 | NM_016587 |
| Monocyte chemotactic protein 3 | Repression | 463 | NP_006264 | 464 | NM_006273 |
| Fatty-acid-Coenzyme A ligase, long-chain 2 | Repression | 465 | BAA00931 | 466 | D10040 |
| Programmed cell death 5/TFAR19 | Repression | 467 | NP_004699 | 468 | NM_004708 |
| Small inducible cytokine A3 | Repression | 469 | AAA36316 | 470 | M23452 |
| Cytochrome c oxidase subunit VIc | Repression | 471 | NP_004365 | 472 | NM_004374 |
| NASP histone-binding prot. | Repression | 473 | NP_002473 | 474 | NM_002482 |
| Ecotropic viral integration site 2A | Repression | 475 | NP_055025 | 476 | NM_014210 |
| Sjogren syndrome antigen B | Repression | 477 | AAA51885 | 478 | J04205 |
| Monocyte chemotactic protein 2 | Repression | 479 | NP_005614 | 480 | NM_005623 |
| GRO2/macrophage inflammatory protein 2a | Repression | 481 | NP_002080 | 482 | NM_002089 |
| Small nuclear ribonucleoprotein SM D 1 | Repression | 483 | NP_008869 | 484 | NM_006938 |
| GRO1/macrophage inflammatory protein 2 precursor | Repression | 485 | NP_001502 | 486 | NM_001511 |
| Lymphocyte adhesion molecule 1 | Repression | 487 | NP_000646 | 488 | NM_000655 |

TABLE 27
cross-references all protein and nucleotide sequences (SEQ ID NOS) that are referenced
herein to accession numbers in public databases available as of 8.12.01.

| | | | Protein | | | |
|---|---|---|---|---|---|---|
| Clone | | | SEQ ID | Protein | Nucleotide | |
| ID | New Name | Old Name | NO: | Accession | SEQ ID NO: | GenBank Locus |
| p1F12 | hypothetical protein FLJ13611 | cDNA FLJ13611 fis, clone | 1 | NP_079217 | 2 | NM_024941 |
| | | PLACE1010802 | | | | |
| p1F2 | hypothetical protein FLJ20037 | hypothetical protein FLJ20037 | 3 | CAB65981 | 4 | NM_017633 |
| p1F10 | hypothetical protein | hypothetical protein | 5 | T46364 | 6 | NM_017593 |
| | DKFZp434P0116 | DKFZp434P0116 | | | | |
| p1F19 | hypothetical protein KIAA0212 | KIAA0212 | 7 | BAA13203 | 8 | NM_014674 |
| p1F8 | hypothetical protein KIAA0914 | KIAA0914 | 9 | NP_055698 | 10 | NM_014883 |
| p1F5 | hypothetical protein FLJ20281 | hypothetical protein FLJ20281 | 11 | XP_008736 | 12 | NM_017742 |
| p1F18 | hypothetical protein KIAA0876 | KIAA0876 | 13 | BAA74899 | 14 | XM_035625 |
| p1F7 | Spectrin, beta, non-erythrocytic 1 | cDNA FLJ13700 fis, clone | 15 | NP_003119 | 16 | NM_003128 |
| | | PLACE2000216 | | | | |
| p1F21 | Hematopoietic Zinc finger protein | DKFZP586G1122 protein | 17 | AAL08625 | 18 | AK024404 |
| p1F9 | hypothetical protein KIAA0742 | Putative zinc finger protein | 19 | NP_060903 | 20 | AB018285 |
| | | LOC55818 | | | | |
| p1E13 | hypothetical protein PRO0823 | hypothetical protein PRO0823 | 21 | AAF71073 | 22 | AF116653 |
| p1D1 | hypothetical protein FLJ10134 | hypothetical protein FLJ10134 | 23 | NP_060474 | 24 | NM_018004 |
| p1D2 | hypothetical protein FLJ10134 | hypothetical protein FLJ10134 | 23 | NP_060474 | 24 | NM_018004 |
| p1D4 | hypothetical protein FLJ20500 | hypothetical protein FLJ20500 | 25 | NP_061931 | 26 | NM_019058 |
| p1D9 | hypothetical protein | DKFZP564D116 protein | 27 | T08708 | 28 | AL050022 |
| | DKFZP564D116 | | | | | |
| p1D12 | hypothetical protein KIAA1376 | KIAA1376 protein | 29 | BAA92614 | 30 | AB037797 |
| p1D15 | TRIP-Br2 | hypothetical protein KIAA0127 | 31 | NP_055570 | 32 | NM_014755 |
| p1D16 | hypothetical protein FLJ20308 | hypothetical protein FLJ20308 | 33 | XP_039852 | 34 | AK000315 |
| p1J13 | hypothetical nuclear factor | hypothetical nuclear factor | 35 | NP_065128 | 36 | NM_020395 |
| | SBBI22 | SBBI22 | | | | |
| p1I22 | hypothetical protein KIAA1429 | DKFZP434I116 protein | 37 | BAA92667 | 38 | AB037850 |
| p1J6 | hypothetical protein FLJ10206 | hypothetical prot. FLJ10206 | 39 | AAH06108 | 40 | NM_018025 |
| p1I5 | hypothetical protein FLJ10815 | hypothetical protein FLJ10815 | 41 | BAA91830 | 42 | NM_018231 |
| p1I13 | hypothetical protein FLJ11100 | hypothetical protein FLJ11100 | 43 | NP_060701 | 44 | NM_018321 |
| p1I17 | hypothetical protein FLJ20644 | hypothetical protein FLJ2064 | 45 | NP_060387 | 46 | NM_017917 |
| p1I15 | hypothetical protein CGI-117 | hypothetical protein HSPC111 | 47 | Q9Y3C1 | 48 | NM_016391 |
| p1I7 | Uridine 5′ monophosphate | hypothetical protein LOC51251 | 49 | NP_057573 | 50 | NM_016489 |
| | hydrolase 1 | | | | | |
| | hypothetical protein KIAA0014 | KIAA0014 | 51 | NP_055480 | 52 | NM_014665 |
| p1I4 | hypothetical protein HSPC196 | hypothetical protein HSPC196 | 53 | NP_057548 | 54 | NM_016464 |
| p1I8 | hypothetical protein FLJ11296 | hypothetical protein FLJ11296 | 55 | XP_004747 | 56 | NM_018384 |
| p1I16 | hypothetical protein KIAA1668 | hypothetical protein bA395L14 | 57 | BAB33338 | 58 | AB051455 |
| p1I11 | SECIS binding protein 2 | cDNA FLJ13016 fis, clone | 59 | AAK57518 | 60 | AF380995 |
| | | NT2RP3000624 | | | | |
| p1E8 | cDNA: FLJ22249 fis, clone | cDNA DKFZp586H0324 clone | 61 | None | 62 | AK025902 |
| | HRC02674 | DKFZp586H0324 | | | | |
| p1E18 | Plexin C1 | Clone 23785 | 63 | NP_005752 | 64 | NM_005761 |
| p1E16 | cDNA DKFZp586E1624 | cDNA DKFZp586E1624 | 65 | None | 66 | AL110152 |
| p1D5 | ERO1 (S. cerevisiae)-like | cDNA FLJ14162 fis, clone | 67 | NP_055399 | 68 | NM_014584 |
| | | NT2RM4002504 | | | | |
| p1D6 | ERO1 (S. cerevisiae)-like | cDNA FLJ14162 fis, clone | 67 | NP_055399 | 68 | NM_014584 |
| | | NT2RM4002504 | | | | |
| p1E12 | hypothetical protein | cDNA DKFZp434E1723 (clone | 69 | XP_05338 | 70 | BC010005 |
| | DKFZP434E1723 | DKFZp434E1723) | | | | |
| p1E10 | cDNA FLJ11041 fis, clone | CDNA FLJ11041 fis, clone | 71 | None | 72 | AK001903 |
| | PLACE 1004405 | PLACE1004405 | | | | |
| p1C21 | Tubulin, beta, 4 | cDNA FLJ10433 fis | 73 | NP_006077 | 74 | NM_006086 |
| | | NT2RP1000478 | | | | |
| pID10 | Insulin induced protein 2 | cDNA DKFZp434O071 | 75 | AAD43048 | 76 | AF125392 |
| p1D13 | Adenylate kinase 3 | cDNA FLJ23313 fis, clone | 77 | NP_037542 | 78 | NM_013410 |
| | | HEP11919 | | | | |
| p1E9 | Novel PI-3-kinase adapter | ESTs | 79 | None | 80 | R62339 |
| p1F1 | EST | ESTs | 81 | None | 82 | AA489477 |
| p1E7 | Novel Metallothionein | ESTs | 83 | None | 84 | R06601 |
| p1E6 | EGL nine (C. elegans) homolog 3 | ESTs | 85 | NP_071356 | 86 | NM_022073 |
| p2B1 | PRAME | ESTs | 87 | NP_006106 | 88 | NM_006115 |
| p1D14 | C1orf12 | ESTs | 89 | NP_071334 | 90 | NM_022051 |
| p1D17 | Semaphorin 4b | ESTs | 91 | BAB21836 | 92 | AB051532 |
| p1P14 | Semaphorin 4b | ESTs | 91 | BAB21836 | 92 | AB051532 |
| p1C24 | SLC25A19 | ESTs | 93 | NP_068380 | 94 | NM_021734 |
| p1D3 | Serine carboxypeptidase 1 | ESTs | 95 | NP_067639 | 96 | NM_021626 |
| p1E14 | Unknown mRNA (schizophrenia- | ESTs | 97 | None | 98 | AY010112 |
| | linked) | | | | | |
| p1E20 | Myo-inositol monophosphatase | ESTs | 99 | AAK52336 | 100 | NM_017813 |
| | A3 | | | | | |
| p2A24 | EST | ESTs | 101 | None | 102 | AA521314 |
| p1E17 | hypothetical protein FLJ31668 | ESTs | 103 | BAB71124 | 104 | AK056230 |
| p1E19 | EST | ESTs | 105 | None | 106 | R51835 |
| p1E15 | cDNA YI27F12 | ESTs | 107 | None | 108 | AF075018 |
| p1E11 | EST | ESTs | 109 | None | 110 | R69248 |
| p1E23 | cDNA FLJ14041 fis, clone | ESTs | 111 | None | 112 | AK024103 |
| | HEMBA1005780 | | | | | |
| p1E21 | Glutamate-cysteine ligase, | ESTs | 113 | NP_002052 | 114 | NM_002061 |
| | modifier subunit | | | | | |
| p1D23 | PTEN | ESTs | 115 | NP_000305 | 116 | NM_000314 |
| p1D24 | EST | ESTs | 117 | None | 118 | T73780 |
| p1D22 | MAX-interacting protein 1 | ESTs | 119 | NP_005953 | 120 | NM_005962 |
| p1E2 | Mannosidase, alpha, class 1A, | ESTs | 121 | NP_005898 | 122 | NM_005907 |
| | member 1 | | | | | |
| p1E1 | EST | ESTs | 123 | None | 124 | AA446361 |
| p1E4 | EST | ESTs | 125 | None | 126 | AA931411 |
| p1D18 | cDNA FLJ13443 fis, clone | ESTs | 127 | None | 128 | AK023505 |
| | PLACE1002853 | | | | | |
| p1D21 | hypothetical protein FLJ22622 | ESTs | 129 | BAB15424 | 130 | NM_025151 |
| p1C22 | CD84-H1 | ESTs | 131 | AAK69052 | 132 | AF275725 |
| p1C23 | hypothetical protien FLJ12832 | ESTs | 133 | XP_043394 | 134 | AK022894 |
| p1D11 | EST | ESTs | 135 | None | 136 | AA251748 |
| p1E3 | CYP1B1 | ESTs | 137 | NP_000095 | 138 | NM_000104 |
| p1D20 | hypothetical protein KIAA1125 | ESTs | 139 | XP_012932 | 140 | AB032951 |
| p1E5 | Hepcidin antimicrobial peptide | ESTs | 141 | NP_066998 | 142 | NM_021175 |
| p1D19 | EST | ESTs | 143 | None | 144 | R68736 |
| p2A15 | Sialyltransferase | cDNA FLJ14028 fis, clone | 145 | NP_006447 | 146 | NM_006456 |
| | | HEMBA1003838 | | | | |
| p1I14 | cDNA DKFZp564D016 | cDNA DKFZp564D016 (clone | 147 | None | 148 | AL050021 |
| | | DKFZp564D016) | | | | |
| p1I2 | cDNA FLJ11302 fis, clone | cDNA FLJ11302 fis, clone | 149 | None | 150 | AK002164 |
| | PLACE1009971 | PLACE1009971 | | | | |
| p1I12 | hypothetical protein MGC4549 | NEDO FLJ10309 fis cl | 151 | XP_032794 | 152 | NM_032377 |
| | | NT2RM2000287 | | | | |
| p1I3 | ELMO2 | Sequence from clone RP11- | 153 | AAL14467 | 154 | XM_012933 |
| | | 394O2 on ch 20 | | | | |
| p1I10 | EST | ESTs | 155 | None | 156 | AA420992 |
| p1H18 | Ubiquitin specific protease 7 | ESTs | 157 | NP_003461 | 158 | NM_003470 |
| p1H24 | Nucleolar phosphoprotein Nopp34 | ESTs | 159 | NP_115766 | 160 | NM_032390 |
| p1E22 | cDNA FLJ13618 fis, clone | ESTs | 161 | None | 162 | AK023680 |
| | PLACE1010925 | | | | | |
| p1H21 | hypothetical protein FLJ13511 | ESTs | 163 | NP_149014 | 164 | NM_033025 |
| p1I1 | Ribosomal RNA intergenic spacer | ESTs | 165 | None | 166 | AA664228 |
| p1H14 | EST | ESTs | 167 | None | 168 | R44397 |
| p1H11 | Carboxypeptidase M | ESTs | 169 | NP_001865 | 170 | NM_001874 |
| p1H17 | EST | ESTs | 171 | None | 172 | W87747 |
| p1H12 | EST | ESTs | 173 | None | 174 | AA973568 |
| p1H7 | EST | ESTs | 175 | None | 176 | T98529 |
| p1H15 | EST | ESTs | 177 | None | 178 | AA022679 |
| p1H20 | EST | ESTs | 179 | None | 180 | H17921 |
| p1H8 | ABL | ESTs | 181 | NP_009297 | 182 | NM_007313 |
| p1H16 | EST | ESTs | 183 | None | 184 | W91958 |
| p1H9 | EST | ESTs | 185 | None | 186 | R63694 |
| p1H23 | hypothetical protein FLJ21094 | ESTs | 187 | AAH14003 | 188 | AK024747 |
| p1H10 | EST | ESTs | 189 | None | 190 | AA909912 |
| p1H6 | EST | ESTs | 191 | None | 192 | T99032 |
| p1H13 | EST | ESTs | 193 | None | 194 | H52503 |
| p1H19 | EST | ESTs | 195 | None | 196 | AA127017 |
| p1G22 | EST | ESTs | 197 | None | 198 | R38647 |
| p1G21 | EST | ESTs | 199 | None | 200 | T87233 |
| p1H1 | hypothetical protein FLJ10826 | ESTs | 201 | BAB14226 | 202 | NM_018233 |
| p1G20 | cDNA YO23H03 | ESTs | 203 | None | 204 | AF075053 |
| p1H5 | hypothetical protein FLJ22690 | ESTs | 205 | NP_078987 | 206 | NM_024711 |
| p1G19 | Mitochondrion sequence | ESTs | 207 | AAH05845 | 208 | BC005845 |
| p1H2 | Fatty acid binding protein 5 | ESTs | 209 | NP_001435 | 210 | NM_001444 |
| p1G18 | Mitochondrion sequence | ESTs | 211 | None | 212 | BC001612 |
| p1H4 | EST | ESTs | 213 | None | 214 | AA679939 |
| p1H3 | EST | ESTs | 215 | None | 216 | AA630167 |
| | BCL2/adenovirus E1B 19kD- | BCL2/adenovirus E1B19 kD- | 217 | NP_004322 | 218 | NM_004331 |
| | interacting protein 3-like | interacting protein 3-like | | | | |
| | SLC2A1 | Solute carrier family 2, member | 219 | NP_006507 | 220 | NM_006516 |
| | | 1 | | | | |
| p1P3 | PDGFB | PDGF beta | 221 | NP_148937 | 222 | NM_033016 |
| p1A8 | Lactate dehydrogenase A | lactate dehydrogenase A | 223 | NP_005557 | 224 | NM_005566 |
| p1A9 | Lactate dehydrogenase A | lactate dehydrogenase A | 223 | NP_005557 | 224 | NM_005566 |
| p1B17 | Tissue factor | Tissue factor | 225 | NP_001984 | 226 | NM_001993 |
| p1O20 | VEGF | Vascular endothelial growth | 227 | NP_003367 | 228 | NM_003376 |
| | | factor | | | | |
| p1B2 | N-myc downstream regulated | RTP/NDRG1 | 229 | NP_006087 | 230 | NM_006096 |
| p1B3 | Proline 4-hydroxylase, alpha | Procollagen-proline 4- | 231 | NP_000908 | 232 | NM_000917 |
| | polypeptide I | hydroxylase alpha 1 | | | | |
| | BCL2/adeno virus E1B-interacting | BCL2/adenovirus E1B- | 233 | NP_004043 | 234 | NM_004052 |
| | protein 3 | interacting protein 3 | | | | |
| p1B18 | Plasminogen activator inhibitor, | Plasminogen activator inhibitor, | 235 | NP_000593 | 236 | NM_000602 |
| | type 1 | type I | | | | |
| p1B19 | Plasminogen activator inhibitor, | Plasminogen activator inhibitor, | 235 | NP_000593 | 236 | NM_000602 |
| | type 1 | type I | | | | |
| p1N17 | COX-2 | Cyclooxygenase 2 | 237 | NP_000954 | 238 | NM_000963 |
| p1A24 | Metallothionein 1H | Metallothionein 1H | 239 | NP_005942 | 240 | NM_005951 |
| | Metallothionein 1L | Metallothionein 1L | 241 | NP_002441 | 242 | NM_002450 |
| p1B1 | Metallothionein 1G | Metallothionein-IG | 243 | NP_005941 | 244 | NM_005950 |
| | Metallothionein 1E (functional) | Metallothionein 1E (functional) | 245 | None | 246 | AA872383 |
| p1A1 | SLC2A3 | Solute carrier family 2, member | 247 | NP_008862 | 248 | NM_006931 |
| | | 3 | | | | |
| p1A2 | SLC2A3 | Solute carrier family 2, member 3 | 247 | NP_008862 | 248 | NM_006931 |
| p1A3 | SLC2A3 | Solute carrier family 2, member | 247 | NP_008862 | 248 | NM_006931 |
| | | 3 | | | | |
| p1A4 | SLC2A3 | Solute carrier family 2, member 3 | 247 | NP_008862 | 248 | NM_006931 |
| p1A15 | Hexokinase-2 | Hexokinase 2 | 249 | NP_000180 | 250 | NM_000189 |
| p1A16 | Hexokinase-2 | Hexokinase 2 | 249 | NP_000180 | 250 | NM_000189 |
| p1A17 | Hexokinase-2 | Hexokinase 2 | 249 | NP_000180 | 250 | NM_000189 |
| p1A18 | Hexokinase-2 | Hexokinase 2 | 249 | NP_000180 | 250 | NM_000189 |
| p1B14 | Interleukin 8 | Interleukin 8 | 251 | NP_000575 | 252 | NM_000584 |
| p1B15 | Interleukin 8 | Interleukin 8 | 251 | NP_000575 | 252 | NM_000584 |
| p1B16 | Interleukin 8 | Interleukin 8 | 251 | NP_000575 | 252 | NM_000584 |
| p1A11 | GAPDH | Glyceraldehyde-3-phosphate | 253 | NP_002037 | 254 | NM_002046 |
| | | dehydrogenase | | | | |
| p1A12 | GAPDH | Glyceraldehyde-3-phosphate | 253 | NP_002037 | 254 | NM_002046 |
| | | dehydrogenase | | | | |
| p1A13 | Phosphoglycerate kinase 1 | Phosphoglycerate kinase 1 | 255 | NP_000282 | 256 | NM_000291 |
| p1A14 | Enolase 1 | Enolase 1 | 257 | NP_001419 | 258 | NM_001428 |
| p1A19 | Aldolase C | aldolase C, fructose- | 259 | NP_005156 | 260 | NM_005165 |
| | | bisphosphate (ALDOC) | | | | |
| p1A20 | Triosephosphate isomerase 1 | Triosephosphate isomerase 1 | 261 | NP_000356 | 262 | NM_000365 |
| | | (TPI1) | | | | |
| p1A22 | Adenylate kinase 3 | Adenylate kinase 3 (AK3) | 263 | NP_037542 | 264 | NM_013410 |
| p1A23 | Metallothionein 2A | Metallothionein-2a | 265 | NP_005944 | 266 | NM_005953 |
| p1B20 | Osteopontin | Osteopontin | 267 | NP_000573 | 268 | NM_000582 |
| p1B21 | Osteopontin | Osteopontin | 267 | NP_000573 | 268 | NM_000582 |
| p1C17 | Granulin | Granulin | 269 | NP_002078 | 270 | NM_002087 |
| p1C18 | Granulin | Granulin | 269 | NP_002078 | 270 | NM_002087 |
| p1D8 | Hypoxia-inducible protein 2 | Hypoxia-inducible protein 2 | 271 | NP_037464 | 272 | NM_013332 |
| p1A10 | Enolase 2 | Enolase 2, (gamma, neuronal) | 273 | NP_001966 | 274 | NM_001975 |
| p1G24 | Glycogen synthase 1 | Glycogen synthase 1 (muscle) | 275 | NP_002094 | 276 | NM_002103 |
| p1G23 | ALCAM | Activated leucocyte cell | 277 | NP_001618 | 278 | NM_001627 |
| | | adhesion molecule | | | | |
| p1G5 | MAX-interacting protein 1 | MAX-interacting protein 1 | 279 | NP_005953 | 280 | NM_005962 |
| p1G7 | EST | Nuclear receptor co-repressor | 281 | None | 282 | BC008022 |
| p2A23 | Chitinase 3-like 2 | Chitinase 3-like 2 | 283 | NP_003991 | 284 | NM_004000 |
| p1G1 | BACH1 | BACH1 transcription factor | 285 | NP_001177 | 286 | NM_001186 |
| p1G15 | Phosphoglucomutase 1 | Phosphoglucomutase 1 | 287 | NP_002624 | 288 | NM_002633 |
| p1F23 | hypothetical protein LOC51014 | CGI-109 protein | 289 | Q9Y3B3 | 290 | AF151867 |
| p1G8 | Sin3-associated polypeptide | SAP30 | 291 | NP_003855 | 292 | NM_003864 |
| p1G13 | ABCA1 | ATP-binding cassette | 293 | NP_005493 | 294 | NM_005502 |
| | | transporter-1 | | | | |
| p1G10 | SEC24 member A | SEC24 protein | 295 | CAA10334 | 296 | AJ131244 |
| p1F24 | Glia-derived nexin | Trinucleotide repeat containing | 297 | AAA35883 | 298 | M17783 |
| | | 3 | | | | |
| p1G2 | Postsynaptic density-95 | Post-synaptic density protein 95 | 299 | NP_001356 | 300 | NM_001365 |
| p1G11 | Tumor protein D52 | Tumor protein D52 | 301 | NP_005070 | 302 | NM_005079 |
| p1G16 | p27, Kip1 | Cyclin-dependent kinase | 303 | NP_004055 | 304 | NM_004064 |
| | | inhibitor p27kip1 | | | | |
| p1G9 | PI-3-kinase, catalytic, beta | phosphoinositide-3-kinase, | 305 | NP_006210 | 306 | NM_006219 |
| | polypeptide | catalytic, beta | | | | |
| p1G4 | SLC5A3 | Solute carrier family 5, member | 307 | AAC39548 | 308 | AF027153 |
| | | 3 | | | | |
| p1G14 | Cytohesin binding protein | PSCDBP | 309 | NP_004279 | 310 | NM_004288 |
| p1A5 | SLC2A5 | Solute carrier family 2, member | 311 | NP_003030 | 312 | NM_003039 |
| | | 5 | | | | |
| p1A6 | SLC2A5 | Solute carrier family 2, member | 311 | NP_003030 | 312 | NM_003039 |
| | | 5 | | | | |
| p1B6 | Adipophilin | Adipophilin | 313 | NP_001113 | 314 | NM_001122 |
| p1B7 | Adipophilin | Adipophilin | 313 | NP_001113 | 314 | NM_001122 |
| p1B8 | Adipophilin | Adipophilin | 313 | NP_001113 | 314 | NM_001122 |
| p1B9 | Adipophilin | Adipophilin | 313 | NP_001113 | 314 | NM_001122 |
| p1G17 | Early development regulator 2 | Early development regulator 2 | 315 | NP_004418 | 316 | NM_004427 |
| p1G3 | B-cell translocation gene 1 | B-cell translocation gene 1, | 317 | NP_001722 | 318 | NM_001731 |
| p1F22 | Sorting nexin 9 | SH3PX1 | 319 | NP_057308 | 320 | NM_016224 |
| p1G12 | Cyclin G2 | Cyclin G2 | 321 | NP_004345 | 322 | NM_004354 |
| p1F11 | hypothetical protein LOC51754 | NAG-5 protein | 323 | XP_049657 | 324 | AL137430 |
| p1F16 | CYP1B1 | Cytochrome P450 IB1 (dioxin- | 325 | NP_000095 | 326 | NM_000104 |
| | | inducible) | | | | |
| p1F14 | Butyrate response factor 1 | Butyrate response factor 1 | 327 | NP_004917 | 328 | NM_004926 |
| p1F17 | P8 protein (candidate of | p8 protein (candidate of | 329 | NP_036517 | 330 | NM_012385 |
| | metastasis 1) | metastasis 1) | | | | |
| p1C1 | CXCR4 | chemokine (C-X-C motif), | 331 | NP_003458 | 332 | NM_003467 |
| | | receptor 4 (CXCR4) | | | | |
| p1C2 | CXCR4 | chemokine (C-X-C motif), | 331 | NP_003458 | 332 | NM_003467 |
| | | receptor 4 (CXCR4) | | | | |
| p1F3 | hypothetical protein XP_017131 | solute carrier family 16, | 333 | XP_017131 | 334 | XM_017131 |
| | | member 6 | | | | |
| p1F20 | Proline-rich protein with nuclear | Proline-rich protein with nuclear | 335 | NP_006804 | 336 | NM_006813 |
| | targeting signal | targeting signal (B4-2) | | | | |
| p1F6 | hypothetical protein hqp0376 | RNA helicase-related protein | 337 | T08745 | 338 | AF078844 |
| p1F4 | CYP1 | Cytochrome P450, subfamily | 339 | NP_000776 | 340 | NM_000785 |
| | | XXVIIB, polypeptide 1 | | | | |
| p1F15 | SHB adaptor protein | SHB adaptor protein | 341 | NP_003019 | 342 | NM_003028 |
| p1F13 | Papillomavirus regulatory factor | Papillomavirus regulatory factor | 343 | NP_061130 | 344 | AK023418 |
| | PRF-1 | (PRF-1) | | | | |
| p1A7 | SLC31A2 | SLC31A2/hCTR1 | 345 | NP_001851 | 346 | NM_001860 |
| p1A21 | UDP-glucose pyrophosphorylase | UDP-glucose | 347 | NP_006750 | 348 | NM_006759 |
| | 2 | pyrophosphorylase 2 (UGP2) | | | | |
| p1B4 | Proline 4-hydroxylase, alpha | Proline 4-hydroxylase, alpha | 349 | NP_004190 | 350 | NM_004199 |
| | polypeptide II | polypeptide II | | | | |
| p1B5 | Proline 4-hydroxylase, alpha | | 349 | NP_004190 | 350 | NM_004199 |
| | polypeptide II | | | | | |
| p1B10 | Stearoyl-CoA desaturase | Stearoyl-CoA desaturase | 351 | NP_005054 | 352 | NM_005063 |
| p1B11 | Stearoyl-CoA desaturase | Stearoyl-CoA desaturase | 351 | NP_005054 | 352 | NM_005063 |
| p1B12 | Stearoyl-CoA desaturase | Stearoyl-CoA desaturase | 351 | NP_005054 | 352 | NM_005063 |
| p1B13 | Diacylglycerol kinase, zeta | Diacylglycerol kinase, zeta | 353 | NP_003637 | 354 | NM_003646 |
| p1B22 | Protease, Serine, 11 | Serine protease 11 | 355 | NP_002766 | 356 | NM_002775 |
| p1B23 | Interleukin 1 receptor antagonist | IL-1 receptor antagonist, | 357 | NP_000568 | 358 | NM_000577 |
| | | alternatively spliced forms | | | | |
| p1B24 | NS1-binding protein | NS1-binding protein | 359 | NP_006460 | 360 | NM_006469 |
| p1C3 | Activin A receptor, type I | Activin A receptor type I | 361 | NP_001096 | 362 | NM_001105 |
| p1C4 | FGF receptor activating protein 1 | FGF receptor activating protein | 363 | NP_055304 | 364 | NM_014489 |
| | | 1 (FRAG1) | | | | |
| p1C5 | Galectin 8 | Galectin-8 | 365 | NP_006490 | 366 | NM_006499 |
| p1C6 | Glucose phosphate isomerase | Glucose 6-phosphate isomerase | 367 | NP_000166 | 368 | NM_000175 |
| p1C7 | D123 | D123 protein | 369 | NP_006014 | 370 | NM_006023 |
| p1C8 | DEC-1 | Dec1. | 371 | NP_003661 | 372 | NM_003670 |
| p1C9 | RAB-8b protein | Rab-8b | 373 | NP_057614 | 374 | NM_016530 |
| p1C10 | Regulator of G-protein signalling | BL34 | 375 | NP_002913 | 376 | NM_002922 |
| | 1 | | | | | |
| p1C11 | Polyubiquitin | Polyubiquitin UbC | 377 | BAA23632 | 378 | AB009010 |
| p1C12 | Integrin, alpha 5 | Integrin alpha 5 | 379 | NP_002196 | 380 | NM_002205 |
| p1C13 | Jk-recombination signal binding | Jk-recombination signal binding | 381 | AAA60258 | 382 | L07872 |
| | protein | protein | | | | |
| p1C14 | Abstrakt | DEAD-box protein abstrakt | 383 | NP_057306 | 384 | NM_016222 |
| p1C15 | High-mobility group protein 2 | High mobility group 2 protein | 385 | NP_002120 | 386 | NM_002129 |
| p1C16 | Decidual protein induced by | Decidual protein induced by | 387 | NP_008952 | 388 | NM_007021 |
| | progesterone | progesterone | | | | |
| p1C19 | GM2 ganglioside activator protein | GM2 ganglioside activator | 389 | NP_000396 | 390 | NM_000405 |
| | | protein. | | | | |
| p1C20 | CNOT8 | CCR4 associated factor 1 | 391 | NP_004770 | 392 | NM_004779 |
| | | (CAF1) | | | | |
| | Similar to Nucleoside | Nucleoside phosphorylase | 393 | None | 394 | AA430382 |
| | phosphorylase | | | | | |
| p1P5 | SCYA2 | Monocyte chemotactic protein 1 | 395 | NP_002973 | 396 | NM_002982 |
| p2L23 | Endothelin 1 | Endothelin 1 | 397 | NP_001946 | 398 | NM_001955 |
| | Similar to Heat shock 70 kD | Heat shock 70 kD protein 4 | 399 | None | 400 | AA633656 |
| | protein 4 | | | | | |
| p1K9 | Lipocortin I | Annexin AI | 401 | NP_000691 | 402 | NM_000700 |
| p1K23 | MYC | p67 myc protein | 403 | NP_002458 | 404 | NM_002467 |
| p1K15 | Alpha-2-macroglobulin | Alpha-2-macroglobulin | 405 | NP_000005 | 406 | NM_000014 |
| p1K8 | SCYA4 | Macrophage inflammatory | 407 | XP_008449 | 408 | XM_008449 |
| | | protein 1b | | | | |
| p1M24 | Sex hormone-binding globulin | Sex hormone-binding globulin | 409 | NP_001031 | 410 | NM_001040 |
| p1K7 | ATP-binding cassette E1 | ATP-binding cassette, sub- | 411 | NP_002931 | 412 | NM_002940 |
| | | family E (OABP), member 1 | | | | |
| p1K16 | CCT6A | Chaperonin/Tcp zeta 1 | 413 | NP_001753 | 414 | NM_001762 |
| p1K18 | Colony-stimulating factor1 | Colony stimulating factor 1 | 415 | AAA52117 | 416 | M37435 |
| | | (macrophage) | | | | |
| p1N1 | GA17 | Dendritic cell protein (GA17) | 417 | NP_006351 | 418 | NM_006360 |
| p1K22 | GPR44 | G protein-coupled receptor 44 | 419 | NP_004769 | 420 | NM_004778 |
| p1K14 | Keratin 6B | Keratin 6A | 421 | NP_005546 | 422 | NM_005555 |
| p1K13 | Lymphocyte adaptor protein | lymphocyte adaptor protein | 423 | NP_005466 | 424 | NM_005475 |
| p1J20 | Neuro-oncological ventral antigen | Neuro-oncological ventral | 425 | NP_002506 | 426 | NM_002515 |
| | 1 | antigen 1 | | | | |
| p1J22 | Neutral sphingomyelinase (N- | N-SMase/FAN | 427 | NP_003571 | 428 | NM_003580 |
| | SMase) activation associated | | | | | |
| | factor | | | | | |
| p1K1 | Cyclophilin F | Peptidylprolyl isomerase F | 429 | NP_005720 | 430 | NM_005729 |
| | | (cyclophilin F) | | | | |
| p1K3 | Pleckstrin | PLECKSTRIN | 431 | NP_002655 | 432 | NM_002664 |
| p1J19 | CFFM4 | High affinity immunoglobulin | 433 | NP_067024 | 434 | NM_021201 |
| | | epsilon receptor beta | | | | |
| p1K2 | CFFM4 | High affinity immunoglobulin | 433 | NP_067024 | 434 | NM_021201 |
| | | epsilon receptor beta | | | | |
| p1K5 | Ribosomal protein L36a | Ribosomal protein L44 | 435 | NP_000992 | 436 | NM_001001 |
| p1J17 | SLC6A1 | Solute carrier family 6 No 1 | 437 | NP_003033 | 438 | NM_003042 |
| p1J18 | Synaptopodin | Synaptopodin | 439 | NP_009217 | 440 | NM_007286 |
| p1J15 | TERA protein | TERA protein | 441 | NP_067061 | 442 | NM_021238 |
| p1K4 | TSC-22 | TGF beta-stimulated protein | 443 | NP_006013 | 444 | NM_006022 |
| | | TSC-22 | | | | |
| p2A14 | EST | Tubulin, beta, 2 | 445 | None | 446 | AA988110 |
| p1J23 | Calgranulin A | Calgranulin A | 447 | NP_002955 | 448 | NM_002964 |
| p1J21 | Replication factor C large subunit | Replication factor C (145 KDa) | 449 | NP_002904 | 450 | NM_002913 |
| p1J24 | Signal recognition particle 19 kD | Signal recognition particle 19 | 451 | NP_003126 | 452 | NM_003135 |
| | | kD protein | | | | |
| p1J16 | cDNA: FLJ23019 fis, clone | Transcription factor SUPT3H | 453 | None | 454 | AK026672 |
| | LNG00916 | | | | | |
| p1J2 | Proteasome subunit, alpha type, 4 | Proteasome component C9 | 455 | NP_002780 | 456 | NM_002789 |
| p1J9 | MAFB | Maf-related leucine zipper | 457 | NP_005452 | 458 | NM_005461 |
| | | homolog | | | | |
| p1J10 | DNCLI2 | dynein, cytoplasmic, light | 459 | NP_006132 | 460 | NM_006141 |
| | | intermediate polypeptide 2 | | | | |
| p1J1 | Chromobox homolog 3 | Heterochromatin-like protein 1 | 461 | NP_057671 | 462 | NM_016587 |
| p1J5 | SCYA7 | Monocyte chemotactic protein 3 | 463 | NP_006264 | 464 | NM_006273 |
| p1J11 | Fatty-acid-Coenzyme A ligase, | Fatty-acid-Coenzyme A ligase, | 465 | NP_066945 | 466 | NM_021122 |
| | long-chain 2 | long-chain 2 | | | | |
| p1J8 | Programmed cell death 5 | Programmed cell death 5/ | 467 | NP_004699 | 468 | NM_004708 |
| | | TFAR19 | | | | |
| p1I20 | SCYA3L | Small inducible cytokine A3 | 469 | CAA36397 | 470 | X52149 |
| p1J3 | Furin | Cytochrome c oxidase subunit | 471 | NP_002560 | 472 | NM_002569 |
| | | VIc | | | | |
| p1J12 | Nuclear autoantigenic sperm | NASP histone-binding prot. | 473 | NP_002473 | 474 | NM_002482 |
| | protein | | | | | |
| p1I23 | Ecotropic viral integration site 2A | Ecotropic viral integration site | 475 | NP_055025 | 476 | NM_014210 |
| | | 2A | | | | |
| p1J7 | Sjogren syndrome antigen B | Sjogren syndrome antigen B | 477 | NP_003133 | 478 | NM_003142 |
| p1I21 | SCYA8 | Monocyte chemotactic protein 2 | 479 | NP_005614 | 480 | NM_005623 |
| p1I19 | GRO2 | GRO2/macrophage | 481 | NP_002080 | 482 | NM_002089 |
| | | inflammatory protein 2a | | | | |
| p1J4 | Small nuclear ribonucleoprotein | Small nuclear ribonucleoprotein | 483 | NP_008869 | 484 | NM_006938 |
| | D1 | SM D 1 | | | | |
| p1I24 | GRO1 | GRO1/macrophage | 485 | NP_001502 | 486 | NM_001511 |
| | | inflammatory protein 2 | | | | |
| | | precursor | | | | |
| p1I18 | Selectin L | Lymphocyte adhesion molecule | 487 | NP_000646 | 488 | NM_000655 |
| | | 1 | | | | |

1. A method for the identification of a gene that is implicated in a specific disease or physiological condition, said method comprising the steps of:
a) comparing:
i) the transcriptome or proteome of a first specialized cell type that is implicated in the disease or condition under first and second experimental conditions; with
ii) the transcriptome or proteome of a second specialized cell type under said first and said second experimental conditions; and
b) identifying as a gene implicated in the disease or physiological condition, a gene that is differentially regulated in the two specialized cell types under the first and second experimental conditions.

2. A method according to claim 1, wherein said specialized cell types are selected from the group consisting of cardiomyocytes, endothelial cells, sensory neurons, motor neurons, CNS neurons, astrocytes, glial cells, schwann cells, mast cells, eosinophils, smooth muscle cells, skeletal muscle cells, pericytes, lymphocytes, tumor cells, monocytes, macrophages, foamy macrophages, granulocytes, synovial cells/synovial fibroblasts and epithelial cells.

3. A method according to claim 1, wherein said first and second experimental conditions differ in respect of the cellular microenvironment, or in respect of exposure to hormones, growth factors, cytokines, chemokines, inflammatory agents, toxins, metabolites, pH, pharmaceutical agents, hypoxia, anoxia, ischemia, imbalance of any plasma-borne nutrient, osmotic stress, temperature, mechanical stress, irradiation, cell-extracellular matrix interactions, cell-cell interactions, accumulations of foreign or pathological extracellular components, intracellular and extracellular pathogens, or a genetic perturbation.

4. A method according to claim 1, wherein the first experimental conditions and second experimental conditions differ in that under the second experimental conditions, the cells are exposed to a physiological stimulus.

5. A method according to claim 4, wherein the physiological stimulus is a physiological, mechanical, temperature, chemical, toxic or pharmaceutical stress.

6. A method according to claim 5, wherein said physiological stress is hypoxia.

7. A method according to claim 1, wherein said first and second experimental conditions are different genetic conditions.

8. A method according to claim 7, wherein said second experimental conditions differ from said first experimental conditions in that the expression of a genetic element is expressed at a different level in said second experimental conditions relative to the level of expression of the genetic element in said first experimental conditions.

9. A method according to claim 8, wherein said genetic element is heterologous to the specialized cell type.

10. A method according to claim 1, wherein the transcriptomes of the specialized cell types are compared by a technique involving hybridization to a nucleic acid array, subtractive mRNA hybridization, the serial analysis of gene expression (SAGE); the selective amplification via biotin- and restriction-mediated enrichment (SABRE); differential display; representational difference analysis (RDA); differential screening of cDNA libraries; Northern blotting; an RNAse protection assay; an S1-nuclease protection assays; RT-PCR; real time RT-PCR (Taq-man); EST sequencing; massively parallel signature sequencing (MPSS); or sequencing by hybridization (SBH).

11. A method according to claim 10, wherein the transcriptomes are compared by hybridization to a nucleic acid array.

12. A substantially purified polypeptide, encoded by a gene implicated in a specific disease or physiological condition by a method according to claim 1.

13. A substantially purified polypeptide, which polypeptide:
i) comprises the amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531;
ii) has an amino acid sequence encoded by a nucleic acid sequence recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, or has an amino acid sequence encoded by a gene identified from an EST recited in any one of these SEQ ID NOS;
iii) is a fragment of a polypeptide according to i) or ii), provided that said fragment retains a biological activity possessed by the full length polypeptide of i) or ii), or has an antigenic determinant in common with the polypeptide of i) or ii); or
iv) is a functional equivalent of a polypeptide of i), ii) or (iii).

14. A polypeptide according to claim 13, wherein said biological activity is a hypoxia-regulated activity.

15. A polypeptide according to claim 14, wherein the expression of the polypeptide is hypoxia-induced.

16. A polypeptide according to claim 15, which polypeptide:
i) comprises the amino acid sequence as recited in any one of SEQ ID NOS: 1, 3, 5, 7, 9, 11, 13, 15, 17, 19, 21, 23, 25, 27, 29, 31, 33, 63, 67, 69, 73, 75, 77, 85, 85a, 87, 89, 91, 93, 95, 99, 103, 113, 115, 119, 121, 129, 131, 133, 137, 139, 141, 527, 529 and 531;
ii) has an amino acid sequence encoded by a nucleic acid sequence recited in any one of SEQ ID NOS: 2, 4, 6, 8, 10, 12, 14, 16, 18, 20, 22, 24, 26, 28, 30, 32, 34, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 528, 530 and 532, or is encoded by a gene identified from an EST recited in any one of these SEQ ID NOS;
iii) is a fragment of a polypeptide according to i) or ii), provided that said fragment retains a biological activity possessed by the full length polypeptide of i) or ii), or has an antigenic determinant in common with the polypeptide of i) or ii); or
iv) is a functional equivalent of a polypeptide of i), ii) or (iii).

17. A polypeptide according to claim 14, wherein the expression of the polypeptide is hypoxia-repressed.

18. A polypeptide according to claim 17, which polypeptide:
i) comprises the amino acid sequence as recited in any one of SEQ ID NOS: 35, 37, 39, 41, 43, 45, 47, 49, 51, 53, 55, 57, 59, 145, 151, 153, 157, 159, 163, 169, 181, 187, 201, 205, 207 and 209;
ii) has an amino acid sequence encoded by a nucleic acid sequence recited in any one of SEQ ID NOS: 36, 38, 40, 42, 44, 46, 48, 50, 52, 54, 56, 58, 60, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214 and 216, or is encoded by a gene identified from an EST recited in any one of these SEQ ID NOS;
iii) is a fragment of a polypeptide according to i) or ii), provided that said fragment retains a biological activity possessed by the full length polypeptide of i) or ii), or has an antigenic determinant in common with the polypeptide of i) or ii); or
iv) is a functional equivalent of a polypeptide of i), ii) or (iii).

19. The polypeptide of claim 13, wherein the functional equivalent according to part iv) is homologous to the amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531 or is homologous to the amino acid sequence encoded by a nucleic acid as recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, and has equivalent biological activity to that possessed by the full length polypeptide of i) or ii).

20. The polypeptide of claim 13, wherein the fragment of part (iii) has greater than 50% sequence identity with the amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531 or with the amino acid sequence that is encoded by a nucleic acid as recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, or with fragments thereof, preferably greater than 60%, 70%, 80%, 90%, 95%, 98% or 99% sequence identity.

21. The polypeptide of claim 13, wherein the fragment of part (iii) has an antigenic determinant in common with a polypeptide according to part i), which consists of 7 or more amino acid residues from the amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531 or the amino acid sequence encoded by a nucleic acid as recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532.

22. A purified and isolated nucleic acid molecule that encodes a polypeptide according to claim 13.

23. A purified nucleic acid molecule according to claim 22, which consists of the nucleic acid sequence as recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, or is a redundant equivalent or fragment thereof.

24. A purified nucleic acid molecule which hydridizes under high stringency conditions with a nucleic acid molecule according to claim 22.

25. A vector comprising a nucleic acid molecule as recited in claim 22.

26. A delivery vehicle comprising a nucleic acid according to claim 22.

27. A host cell transformed with a vector according to claim 25.

28. An antagonist ligand which binds specifically to a polypeptide according to claim 13, wherein the ligand optionally inhibits the hypoxia-induced activity of said polypeptide.

29. An agonist ligand which binds specifically to a polypeptide according to claim 13, preferably a ligand which augments or potentiates a hypoxia-induced activity of said polypeptide.

30. A ligand according to claim 28, which is an antibody.

31. A ligand according to claim 28, which is a peptide, a peptidomimetic, or a drug molecule, a small natural or synthetic organic molecule of up to 2000 Da, or a small natural or synthetic organic molecule of 800 Da or less.

32. A method for treating or diagnosing a disease, which comprises administering a polypeptide according to claim 13, a nucleic acid molecule according to claim 22, a vector according to claim 25 or a ligand according to claim 30 to a subject.

33. The method of claim 32, wherein said disease is a hypoxia-regulated condition.

34. The method of claim 33, wherein said hypoxia-regulated condition is tumourigenesis, angiogenesis, apoptosis, inflammation, erythropoiesis, or a biological response to hypoxia conditions selected from the group consisting of glycolysis, gluconeogenesis, glucose transportation, catecholamine synthesis, iron transport, and nitric oxide synthesis.

35. A substantially purified polypeptide, which polypeptide:
i) comprises the amino acid sequence as recited in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 63, 67, 69, 73, 75, 77, 85, 85a, 87, 89, 91, 93, 95, 99, 103, 113, 115, 119, 121, 129, 131, 133, 137, 139, 141, 145, 151, 153, 157, 159, 163, 169, 181, 187, 201, 205, 207, 209, 527, 529 and 531 or SEQ ID NOS: 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485 and 487;
ii) has an amino acid sequence encoded by a nucleic acid sequence recited in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 86a, 88, 90, 90a, 92, 92a, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 528, 530 and 532, or has an amino acid sequence encoded by a gene identified from an EST recited in any one of these SEQ ID NOS;
iii) is a fragment of a polypeptide according to i) or ii), provided that said fragment retains a biological activity possessed by the full length polypeptide of i) or ii), or has an antigenic determinant in common with the polypeptide of i) or ii); or
iv) is a functional equivalent of a polypeptide of i), ii) or (iii).
for use in the diagnosis or therapy of the disease or abnormal physiological condition that is affected by hypoxia, wherein said disease or abnormal physiological condition that is treated by hypoxia is selected from the group consisting of cancer, ischaemic conditions, reperfusion injury, retinopathy, neonatal stress, preeclapmsia, atherosclerosis, inflammatory conditions, wound healing, tumourigenesis, angiogenesis, apoptosis, inflammation, erythropoiesis, hair loss, or the biological response to hypoxia conditions, including processes such as glycolysis, gluconeogenesis, glucose transportation, catecholamine synthesis, iron transport and nitric oxide synthesis.

36. A purified and isolated nucleic acid molecule that encodes a polypeptide as recited in claim 35, for use in the diagnosis or therapy of for use in the diagnosis or therapy of a disease or abnormal physiological condition that is affected by hypoxia, wherein said disease or abnormal physiological condition that is affected by hypoxia is selected from the group consisting of cancer, ischaemic conditions, reperfusion injury, retinopathy, neonatal stress, preeclapmsia, atherosclerosis, inflammatory conditions, wound healing, tumourigenesis, angiogenesis, apoptosis, inflammation, erythropoiesis, and hair loss.

37. A purified nucleic acid molecule as recited in claim 36, which consists of the nucleic acid sequence as recited in any one of SEQ ID NOS: 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486 and 488, or which is a redundant equivalent or fragment thereof, for use in the diagnosis or therapy of a disease or abnormal physiological condition that is affected by hypoxia, wherein said disease or abnormal physiological condition that is affected by hypoxia is selected from the group consisting of cancer, ischaemic conditions, reperfusion injury, retinopathy, neonatal stress, preeclapmsia, atherosclerosis, inflammatory conditions, wound healing, tumourigenesis, angiogenesis, apoptosis, inflammation, erythropoiesis, and hair loss.

38. A purified nucleic acid molecule which hydridizes under high stringency conditions with a nucleic acid molecule as recited in claim 36, for use in the diagnosis or therapy of a disease or abnormal physiological condition that is affected by hypoxia, wherein said disease or abnormal physiological condition that is affected by hypoxia is selected from the group consisting of cancer, ischaemic conditions, reperfusion injury, retinopathy, neonatal stress, preeclapmsia, atherosclerosis, inflammatory conditions, wound healing, tumourigenesis, angiogenesis, apoptosis, inflammation, erythropoiesis, and hair loss.

39. A vector comprising a nucleic acid molecule as recited in claim 36, for use in the diagnosis or therapy of a disease or abnormal physiological condition that is affected by hypoxia, wherein said disease or abnormal physiological condition that is affected by hypoxia is selected from the group consisting of cancer, ischaemic conditions, reperfusion injury, retinopathy, neonatal stress, preeclapmsia, atherosclerosis, inflammatory conditions, wound healing, tumourigenesis, angiogenesis, apoptosis, inflammation, erythropoiesis, and hair loss.

40. A ligand which binds specifically to, and which optionally inhibits the hypoxia-induced activity of, a polypeptide as recited in claim 35, for use in the diagnosis or therapy of tumourigenesis, angiogenesis, apoptosis, the biological response to hypoxia conditions, or a hypoxic-associated pathology.

41. A pharmaceutical composition suitable for modulating hypoxia, ischaemia, or hypoxia and ischaemia, comprising a therapeutically-effective amount of a polypeptide as recited in claim 13, a nucleic acid molecule according to claim 22, a vector according to claim 25, or a ligand according to claim 30, in conjunction with a pharmaceutically-acceptable carrier.

42. A pharmaceutical composition according to claim 41, wherein said pharmaceutically-acceptable carrier is a liposome.

43. A vaccine composition comprising a polypeptide as recited in claim 13, a nucleic acid molecule as recited in claim 22, or a vector according to claim 25.

44. A method of treating a disease in a patient in need of such treatment by administering to a patient a therapeutically effective amount of a polypeptide as recited in claim 13, an antagonist of said polypeptide, or a nucleic acid molecule as recited in claim 22.

45. A method of regulating tumourigenesis, angiogenesis, apoptosis, the biological response to hypoxia conditions, or a hypoxic-associated pathology in a patient in need of such treatment by administering to a patient a therapeutically effective amount of a polypeptide according to claim 13, a nucleic acid molecule according to claim 22, a vector according to claim 25, a ligand according to claim 30.

46. A method according to claim 45, wherein, for diseases in which the expression of the natural gene or the activity of the polypeptide is lower in a diseased patient when compared to the level of expression or activity in a healthy patient, the polypeptide, nucleic acid molecule, ligand, compound or composition administered to the patient is an agonist.

47. A method according to claim 45, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

48. A method of monitoring the therapeutic treatment of a disease or physiological condition in a patient, comprising monitoring over a period of time the level of expression or activity of polypeptide according to claim 13, or a nucleic acid molecule according to claim 22, in tissue from said patient, wherein altering said level of expression or activity over the period of time towards a control level is indicative of regression of said disease.

49. A method of providing a hypoxia regulating gene, an apoptotic or an angiogenesis regulating gene by administering directly to a patient in need of such therapy an expressible vector comprising expression control sequences operably linked to a nucleic acid molecule recited in claim 22.

50. A method of diagnosing a hypoxia-regulated condition in a patient, comprising assessing the level of expression of a natural gene encoding a polypeptide according to claim 13, or assessing the activity of such a polypeptide, in tissue from said patient and comparing said level of expression or activity to a control level, wherein a level that is different to said control level is indicative of the hypoxia-related condition.

51. A method according to claim 50 that is carried out in vitro.

52. A method according to claim 50, which comprises: (a) contacting a ligand according to claim 30 with a biological sample under conditions suitable for the formation of a ligand-polypeptide complex; and (b) detecting said complex.

53. A method according to claim 50, comprising:
a) contacting a sample of tissue from the patient with a nucleic acid probe under stringent conditions that allow the formation of a hybrid complex between a nucleic acid molecule according to claim 22 and the probe;
b) contacting a control sample with said probe under the same conditions used in step a); and
c) detecting the presence of hybrid complexes in said samples;
whereby detection of levels of the hybrid complex in the patient sample that differ from levels of the hybrid complex in the control sample is indicative of the hypoxia-related condition.

54. A method according to claim 50, comprising:
a) contacting a sample of nucleic acid from the tissue of the patient with a nucleic acid primer under stringent conditions that allows the formation of a hybrid complex between a nucleic acid molecule according to claim 22 and the primer;
b) contacting a control sample with said primer under the same conditions used in step a);
c) amplifying the sampled nucleic acid; and
d) detecting the level of amplified nucleic acid from both patient and control samples;
whereby detection of levels of the amplified nucleic acid in the patient sample that differ significantly from levels of the amplified nucleic acid in the control sample is indicative of the hypoxia-related condition.

55. A method according to claim 50, comprising:
a) obtaining a tissue sample from a patient being tested for the hypoxia-related condition;
b) isolating a nucleic acid molecule according to claim 22 from said tissue sample; and
c) diagnosing the patient for disease by detecting the presence of a mutation which is associated with the hypoxia-related condition in the nucleic acid molecule as an indication of the hypoxia-related condition.

56. The method of claim 55, further comprising amplifying the nucleic acid molecule to form an amplified product and detecting the presence or absence of a mutation in the amplified product.

57. A method according to claim 48, wherein said disease is cancer, ischaemic conditions, reperfusion injury, retinopathy, neonatal stress, preeclapmsia, atherosclerosis, inflammatory conditions, wound healing, tumourigenesis, angiogenesis, apoptosis, inflammation, erythropoiesis, or hair loss.

58. A method according to claim 57, wherein said hypoxia or ischaemia-related tissue damage is due to a disorder of the cerebral, coronary or peripheral circulation.

59. A method according to claim 48, wherein the tissue is a cancer tissue.

60. A method for the identification of a compound that is effective in the treatment, diagnosis, or treatment and diagnosis of disease, comprising contacting a polypeptide according to claim 13, or a nucleic acid molecule according to claim 22 with one or more compounds suspected of possessing binding affinity for said polypeptide or nucleic acid molecule, and selecting a compound that binds specifically to said nucleic acid molecule or polypeptide.

61. A method for the identification of a compound that is effective in the treatment, diagnosis, or treatment and diagnosis of disease, comprising contacting a cell or cell membrane preparation comprising a polypeptide according to claim 13, or a nucleic acid molecule according to claim 22 with one or more candidate compounds and detecting the degree of compound binding, or the stimulation or inhibition of a functional response in said cell or cell membrane.

62. A method according to claim 61, wherein said polypeptide comprises the amino acid sequence recited in SEQ ID NO: 85 or 85a, or said nucleic acid molecule comprises the nucleotide sequence recited in SEQ ID NO: 86 or 86a, and the functional response is the degree of prolyl 4-hydroxylase activity evident in said cell or cell membrane.

63. A method according to claim 62, wherein said functional response is the degree of prolyl 4-hydroxylation of HIF-1α in said cell or cell membrane.

64. A method according to claim 61, wherein said polypeptide comprises the amino acid sequence recited in SEQ ID NO:85 or 85a, or said nucleic acid molecule comprises the nucleotide sequence recited in SEQ ID NO: 86 or 86a, and the functional response is the ability of said polypeptide to interact with HIF-1α.

65. A method according to claim 61, wherein said polypeptide comprises the amino acid sequence recited in SEQ ID NO:527, 529 or 531, or said nucleic acid molecule comprises the nucleotide sequence recited in SEQ ID NO: 528, 530 or 532, and the functional response is the degree of PI3-kinase activity evident in said cell or cell membrane.

66. A method according to claim 61, wherein said polypeptide comprises the amino acid sequence recited in SEQ ID NO:527, 529 or 531, or said nucleic acid molecule comprises the nucleotide sequence recited in SEQ ID NO: 528, 530 or 532, and the functional response is the ability of said polypeptide to interact with a tyrosine kinase, the p85 subunit of PI3-kinase in said cell or cell membrane, or a combination thereof.

67. A compound identified or identifiable by a method according to claim 60.

68. A method according to claim 62, wherein said candidate compounds is a natural or modified substrate, an enzyme, a receptor, a small organic molecule, a small natural or synthetic organic molecule of up to 2000 Da, a small natural or synthetic organic molecule of 800 Da or less, a peptidomimetic, an inorganic molecule, a peptide, a polypeptide, an antibody, or a structural or functional mimetic of the forgoing.

69. A kit useful for diagnosing disease comprising a first container comprising a nucleic acid probe that hybridizes under stringent conditions with a nucleic acid molecule according to claim 22; a second container comprising primers useful for amplifying said nucleic acid molecule; and instructions for using the probe and-primers for facilitating the diagnosis of disease.

70. The kit of claim 69, further comprising a third container comprising an agent for digesting unhybridised RNA.

71. An array of at least two nucleic acid molecules, wherein each of said nucleic acid molecules either corresponds to the sequence of, is complementary to the sequence of, or hybridizes specifically to a nucleic acid molecule according to claim 22.

72. An array according to claim 71, which contains nucleic acid molecules that either correspond to the sequence of, are complementary to the sequence of, or hybridise specifically to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 90a, 91, 92, 92a, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294 or 295 of the nucleic acid molecules implicated in a hypoxia-regulated condition as recited in claim 22.

73. An array according to 71, wherein said nucleic acid molecules consist of between twelve and two thousand nucleotides.

74. An array of antibodies, comprising at least two different antibody species, wherein each antibody species is immunospecific with a polypeptide implicated in a hypoxia-regulated condition as recited in claim 13.

75. An array of polypeptides, comprising at least two polypeptide species as recited in claim 13, wherein each polypeptide species is implicated in a hypoxia-regulated condition, or is a functional equivalent variant or fragment thereof.

76. A kit comprising an array of nucleic acid molecules according to claim 71.

77. A kit comprising one or more antibodies that bind to a polypeptide as recited in claim 13; and a reagent useful for the detection of a binding reaction between said antibody and said polypeptide.

78. A transgenic or knockout non-human animal that has been transformed to express higher, lower or absent levels of a polypeptide according to claim 13.

79. A method for screening for a compound effective to treat disease, by contacting a non-human transgenic animal according to claim 78 with a candidate compound and determining the effect of the compound on the disease or physiological condition of the animal.

80. A protein complex comprising the EGLN3 (BAB15101; SEQ ID NO: 85), the EGLN3 splice variant (SEQ ID NO: 85a), EGLN1 (c1orf12; AAG34568; SEQ ID NO: 89) or CAB81622 polypeptide, complexed with a polypeptide selected from the group consisting of Hif1alpha, Hif2alpha, Hif3alpha, a member of the PAS-domain transcription factor family, p53 and a protein binding partner that is equivalent to the beta subunit of P4H.

81. A protein complex according to claim 80, which is a dimer or tetramer complex.

82. A protein complex comprising a Hu.BCAP-A polypeptide or a functional equivalent thereof, a tyrosine kinase protein, a PI3-kinase protein, or combinations thereof.

83. A substantially purified polypeptide comprising the consensus sequence:
KAMVACYPGNGTGYVRHVDNPNGDGRCITCIYYLNKNWDAKLHGGILRIFPEGKS FIADVEPIFDRLLFFWSDRRNPHEVQPSYATRYAMTVWYFDAEERAEAKKK, or a variant thereof.

84. A substantially purified polypeptide according to claim 80, for use in the diagnosis or treatment of a hypoxia-related disease or condition.
